(19) United States
(12) Patent Application Publication
(10) Pub. No.: US 2014/0221361 A1
(43) Pub. Date: Aug. 7, 2014

(54) C-19 MODIFIED TRITERPENOIDS WITH HIV MATURATION INHIBITORY ACTIVITY

(71) Applicant: BRISTOL-MYERS SQUIBB COMPANY, Princeton, NJ (US)

(72) Inventors: Jacob Swidorski, Southington, CT (US); Brian Lee Venables, Durham, CT (US); Zheng Liu, Beacon Falls, CT (US); Ny Sin, East Hampton, CT (US); Nicholas A. Meanwell, East Hampton, CT (US); Alicia Regueiro-Ren, Middletown, CT (US)

(21) Appl. No.: 14/172,389

(22) Filed: Feb. 4, 2014

Related U.S. Application Data
(60) Provisional application No. 61/761,403, filed on Feb. 6, 2013.

(51) Int. Cl. C07J 63/00; A61K 31/58; A61K 31/56; A61K 45/06

(52) U.S. Cl. CPC C07J 63/008; A61K 45/06; A61K 31/58; A61K 31/56
USPC 514/227.8; 544/154; 514/237.8; 562/451; 514/567; 562/442; 514/566; 562/443; 514/563; 514/239.2; 544/141; 514/235.5; 544/139; 514/235.8; 544/58.2; 514/227.5

(57) ABSTRACT

Compounds having drug and bio-affecting properties, their pharmaceutical compositions and methods of use are set forth. In particular, C-19 modified triterpenoids that possess unique antiviral activity are provided as HIV maturation inhibitors, as represented by compounds of Formulas I and II:

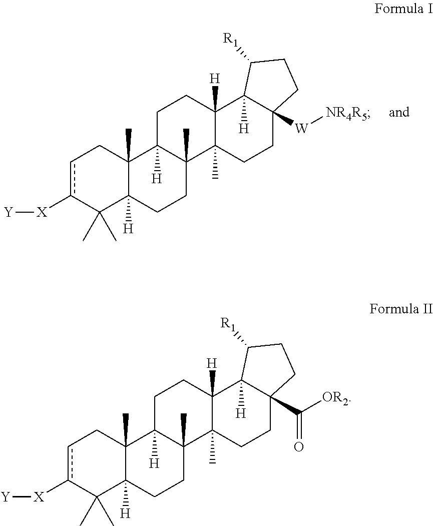

These compounds are useful for the treatment of HIV and AIDS.

### CROSS REFERENCE TO RELATED APPLICATION

This application claims the priority of U.S. Provisional Application Ser. No. 61/761,403 filed Feb. 6, 2013 which is herein incorporated by reference.

### FIELD OF THE INVENTION

The present invention relates to novel compounds useful against HIV, and more particularly, to compounds derived from betulinic acid and other structurally-related compounds which are useful as HIV maturation inhibitors, and to pharmaceutical compositions containing same, as well as to methods for their preparation.

### BACKGROUND OF THE INVENTION

HIV-1 (human immunodeficiency virus-1) infection remains a major medical problem, with an estimated 45-50 million people infected worldwide at the end of 2010. The number of cases of HIV and AIDS (acquired immunodeficiency syndrome) has risen rapidly. In 2005, approximately 5.0 million new infections were reported, and 3.1 million people died from AIDS. Currently available drugs for the treatment of HIV include nucleoside reverse transcriptase (RT) inhibitors or approved single pill combinations: zidovudine (or AZT or RETROVIR®), didanosine (or VIDEX®), stavudine (or ZERIT®), lamivudine (or 3TC or EPIVIR®), zalcitabine (or DDC or HIVID®), abacavir succinate (or ZIAGEN®), Tenofovir disoproxil fumarate salt (or VIREAD®), emtricitabine (or FTC-EMTRIVA®), COMBIVIR® (contains -3TC plus AZT), TRIZIVIR® (contains abacavir, lamivudine, and zidovudine), EPZICOM® (contains abacavir and lamivudine), TRUVADA® (contains VIREAD® and)EMTRIVA®; non-nucleoside reverse transcriptase inhibitors: nevirapine (or VIRAMUNE®), delavirdine (or RESCRIPTOR®) and efavirenz (or SUSTIVA®), ATRIPLA® (TRUVADA®+SUSTIVA®), and etravirine, and peptidomimetic protease inhibitors or approved formulations: saquinavir, indinavir, ritonavir, nelfinavir, amprenavir, lopinavir, KALETRA® (lopinavir and Ritonavir), darunavir, atazanavir (REYATAZ®) and tipranavir (APTIVUS®) and cobicistat, and integrase inhibitors such as raltegravir (ISENTRESS®), and entry inhibitors such as enfuvirtide (T-20) (FUZEON®) and maraviroc (SELZENTRY®).

Each of these drugs can only transiently restrain viral replication if used alone. However, when used in combination, these drugs have a profound effect on viremia and disease progression. In fact, significant reductions in death rates among AIDS patients have been recently documented as a consequence of the widespread application of combination therapy. However, despite these impressive results, 30 to 50% of patients may ultimately fail combination drug therapies. Insufficient drug potency, non-compliance, restricted tissue penetration and drug-specific limitations within certain cell types (e.g. most nucleoside analogs cannot be phosphorylated in resting cells) may account for the incomplete suppression of sensitive viruses. Furthermore, the high replication rate and rapid turnover of HIV-1 combined with the frequent incorporation of mutations, leads to the appearance of drug-resistant variants and treatment failures when sub-optimal drug concentrations are present. Therefore, novel anti-HIV agents exhibiting distinct resistance patterns, and favorable pharmacokinetic as well as safety profiles are needed to provide more treatment options. Improved HIV fusion inhibitors and HIV entry coreceptor antagonists are two examples of new classes of anti-HIV agents further being studied by a number of investigators.

HIV attachment inhibitors are a further subclass of antiviral compounds that bind to the HIV surface glycoprotein gp120, and interfere with the interaction between the surface protein gp120 and the host cell receptor CD4. Thus, they prevent HIV from attaching to the human CD4 T-cell, and block HIV replication in the first stage of the HIV life cycle. The properties of HIV attachment inhibitors have been improved in an effort to obtain compounds with maximized utility and efficacy as antiviral agents. In particular, U.S. Pat. No. 7,354,924 and U.S. Pat. No. 7,745,625 are illustrative of HIV attachment inhibitors.

Another emerging class of compounds for the treatment of HIV are called HIV maturation inhibitors. Maturation is the last of as many as 10 or more steps in HIV replication or the HIV life cycle, in which HIV becomes infectious as a consequence of several HIV protease-mediated cleavage events in the gag protein that ultimately results in release of the capsid (CA) protein. Maturation inhibitors prevent the HIV capsid from properly assembling and maturing, from forming a protective outer coat, or from emerging from human cells. Instead, non-infectious viruses are produced, preventing subsequent cycles of HIV infection.

Certain derivatives of betulinic acid have now been shown to exhibit potent anti-HIV activity as HIV maturation inhibitors. For example, U.S. Pat. No. 7,365,221 discloses monoacylated betulin and dihydrobetuline derivatives, and their use as anti-HIV agents. As discussed in the '221 reference, esterification of betulinic acid (1) with certain substituted acyl groups, such as 3′,3′-dimethylglutaryl and 3′,3′-dimethylsuccinyl groups produced derivatives having enhanced activity (Kashiwada, Y., et al., J. Med. Chem. 39:1016-1017 (1996)). Acylated betulinic acid and dihydrobetulinic acid derivatives that are potent anti-HIV agents are also described in U.S. Pat. No. 5,679,828. Esterification of the hydroxyl in the 3 carbon of betulin with succinic acid also produced a compound capable of inhibiting HIV-1 activity (Pokrovskii, A. G., et al., Khimiya y Interesakh Ustoichivogo Razvitiya, Vol. 9, No. 3, pp. 485-491 (2001)).

Other references to the use of treating HIV infection with compounds derived from betulinic acid include US 2005/0239748 and US 2008/0207573, as well as WO2006/053255, WO2009/100532 and WO2011/007230.

One HIV maturation compound that has been in development has been identified as Bevirimat or PA-457, with the chemical formula of C₃₆H₅₆O₆ and the IUPAC name of 3β-(3-carboxy-3-methyl-butanoyloxy) lup-20(29)-en-28-oic acid.

Reference is also made herein to the applications by Bristol-Myers Squibb entitled “MODIFIED C-3 BETULINIC ACID DERIVATIVES AS HIV MATURATION INHIBITORS” U.S. Ser. No. 13/151,706 filed on Jun. 2, 2011 (now US 2012-0142707) and “C-28 AMIDES OF MODIFIED C-3 BETULINIC ACID DERIVATIVES AS HIV MATURATION INHIBITORS” U.S. Ser. No. 13/151,722, filed on Jun. 2, 2011 (now US 2012-0142653). Reference is also made to the application entitled “C-28 AMINES OF C-3 MODIFIED BETULINIC ACID DERIVATIVES AS HIV MATURATION INHIBITORS” U.S. Ser. No. 13/359,680, filed on Jan. 27, 2012 (now U.S. 2013-0029954). In addition, reference is made to the application entitled “C-17 AND C-3 MODIFIED TRITERPENOIDS WITH HIV MATURATION INHIBITORY ACTIVITY” U.S. Ser. No. 13/359,727 filed on Jan. 27, 2012 (now U.S. 2013-0035318), and to the application entitled “C-17 BICYCLIC AMINES OF TRITERPENOIDS WITH HIV MATURATION INHIBITORY ACTIVITY”, U.S. Ser. No. 13/799,479 filed on Mar. 13, 2013 (now U.S. 2013-0296554).

What is now needed in the art are new compounds which are useful as HIV maturation inhibitors, as well as new pharmaceutical compositions containing these compounds.

### SUMMARY OF THE INVENTION

The present invention provides compounds of Formulas I and II below, including pharmaceutically acceptable salts thereof, their pharmaceutical formulations, and their use in patients suffering from or susceptible to a virus such as HIV. The compounds of Formulas I and II are effective antiviral agents, particularly as inhibitors of HIV. They are useful for the treatment of HIV and AIDS.

One embodiment of the present invention is directed to a compound, including pharmaceutically acceptable salts thereof, which is selected from the group of:

a compound of formula I

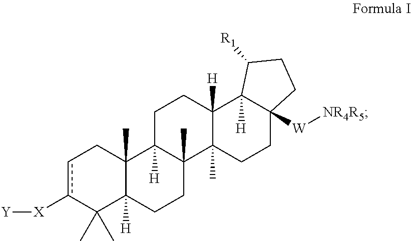

and
a compound of formula II

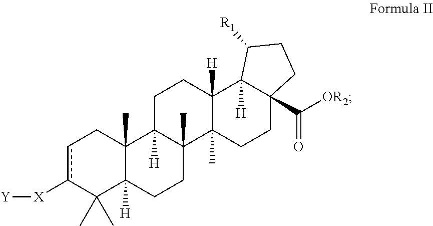

wherein X is selected from the group of phenyl, heteroaryl ring, C₄₋₈ cycloalkyl, C₄₋₈ cycloalkenyl, C₄₋₉ spirocycloalkyl, C₄₋₉ spirocycloalkenyl, C₄₋₈ oxacycloalkyl, C₄₋₈ dioxacycloalkyl, C₆₋₈ oxacycloalkenyl, C₆₋₈ dioxacycloalkenyl, C₆ cyclodialkenyl, C₆ oxacyclodialkenyl, C₆₋₉ oxaspirocycloalkyl and C₆₋₉ oxaspirocycloalkenyl ring;
and further wherein X is substituted with A, wherein A is at least one member selected from the group of —H, -halo, -hydroxyl, —C₁₋₆ alkyl, —C₁₋₆ alkoxy, —C₁₋₆ alkyl-Q₁, -alkylsubstituted C₁₋₆ alkyl-Q₁, —CN, —CF₂Q₁, —NR₂R₂, —COOR₂ and —CONR₂R₂,
wherein Q₁ is selected from the group of aryl, heteroaryl, substituted heteroaryl, —OR₂, —COOR₃, —NR₂R₂, —SO₂R₇, —CONHSO₂R₃, and —CONHSO₂NR₂R₂;
Y is selected from the group of —COOR₂, —C(O)NR₂SO₂R₃, —C(O)NHSO₂NR₂R₂, —NR₂SO₂R₂, —SO₂NR₂R₂, —C₃₋₆ cycloalkyl-COOR₂, —C₂₋₆ alkenyl-COOR₂, —C₂₋₆ alkynyl-COOR₂, —C₁₋₆ alkyl-COOR₂, -alkylsubstituted C₁₋₆ alkyl, —COOR₂, CF₂—COOR₂, —NHC(O)(CH₂)ₙ—COOR₂, —SO₂NR₂C(O)R₂, _tetrazole, and —CONHOH, wherein n=1-6;
R₁ is selected from the group of:

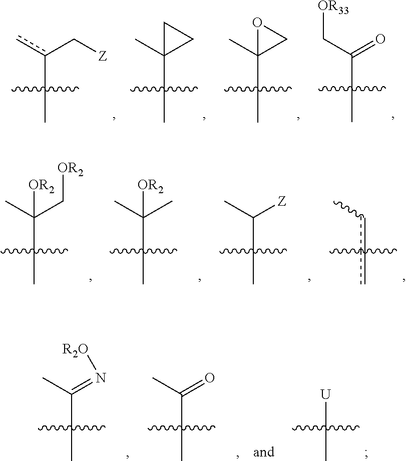

W is absent, or is —CH₂ or —CO;
Z is selected from the group of —NR₂₈R₂₉, —OR₃₀, —COOR₂, —CONR₁₈R₁₉, F, Cl, Br, and I;
U is selected from the group of —NR₂₈R₂₉, —OR₃₀, —COOR₂, —CONR₁₈R₁₉, F, Cl, Br, I, aryl and heteroaryl;
R₂ is selected from the group of —H, benzyl, —C₁₋₆ alkyl, -alkylsubstituted C₁₋₆ alkyl and -arylsubstituted C₁₋₆ alkyl;
R₃ is benzyl, —C₁₋₆ alkyl or -alkylsubstituted C₁₋₆ alkyl;
R₄ is selected from the group of —H, —C₁₋₆ alkyl, —C₁₋₆ alkyl-C(OR₃)₂—C₃₋₆ cycloalkyl, —C₁₋₆ substituted alkyl, —C₁₋₆ alkyl-C₃₋₆ cycloalkyl, —C₁₋₆ alkyl-Q₂, —C₁₋₆ alkyl-C₃₋₆ cycloalkyl-Q₂, aryl, heteroaryl, substituted heteroaryl, —COR₆, —COCOR₆, —SO₂R₇, —SO₂NR₂R₂,

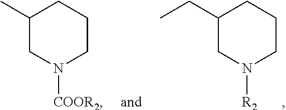

wherein Q₂ is selected from the group of heteroaryl, substituted heteroaryl, F, Cl, Br, I, —CF₃, —OR₂, —COOR₂, —NR₈R₉, —CONR₁₀R₁₁ and —SO₂R₇;
R₅ is selected from the group of —H, —C₁₋₆ alkyl, —C₃₋₆ cycloalkyl, —C₁₋₆ alkylsubstituted alkyl, —C₁₋₆ alkyl-NR₈R₉, —COR_{E}, —COCOR₆, —SO₂R₂ and —SO₂NR₂R₂;
with the proviso that R₄ or R₅ cannot be —COR_{E} or —COCOR₆ when W is CO;
with the further proviso that only one of R₄ or R₅ can be selected from the group of —COR₆, —COCOR₆, —SO₂R₂ and —SO₂NR₂R₂;
or when W is absent or is CH₂, then R₄ and R₅ can be taken together with the adjacent N to form

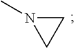

R₆ is selected from the group of —C₁₋₆ alkyl, —C₁₋₆ alkyl-substitutedalkyl, —C₃₋₆ cycloalkyl, —C₃₋₆ substitutedcycloalkyl-Q₃, —C₁₋₆ alkyl-Q₃, —C₁₋₆ alkyl-substitutedalkyl-Q₃, —C₃₋₆ cycloalkyl-Q₃, aryl-Q₃, —NR₁₃R₁₄, and —OR₁₅;
wherein Q₃ is selected from the group of aryl, heteroaryl, substituted heteroaryl, —OR₂, —COOR₂, —NR₈R₉, SO₂R₇, —CONHSO₂R₃, and —CONHSO₂NR₂R₂;
R₇ is selected from the group of —C₁₋₆ alkyl, —C₁₋₆ substituted alkyl, —C₃₋₆ cycloalkyl, —CF₃, aryl, and heteroaryl;
R₈ and R₉ are independently selected from the group of —H, —C₁₋₆ alkyl, —C₁₋₆ substituted alkyl, aryl, heteroaryl, substituted aryl, substituted heteroaryl, and —C₁₋₆ alkyl-Q₂;
R₈ can also be —COOR₃;
R₈ and R₉ can also be independently selected from the group of

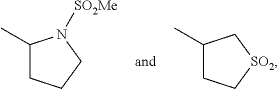

or R₈ and R₉ are taken together with the adjacent N to form a cycle selected from the group of:

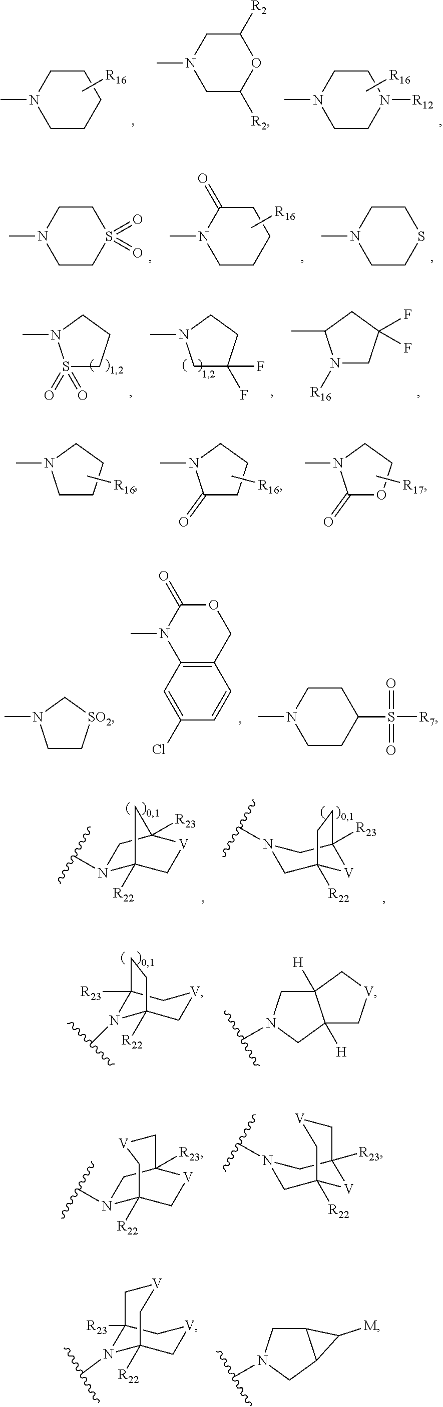

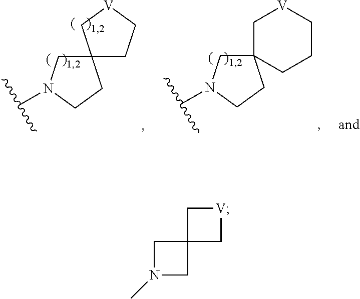

V is selected from the group of —CR₂₄R₂₅, —SO₂, —O and —NR₁₂;
M is selected from the group of —CHR₂₄R₂₅, —NR₂₆R₂₇, —SO₂R₇, —SO₂NR₃R₃ and —OH;
R₁₀ and R₁₁ are independently selected from the group of —H, —C₁₋₆ alkyl, —C₁₋₆ substituted alkyl and —C₃₋₆ cycloalkyl,
or R₁₀ and R₁₁ are taken together with the adjacent N to form a cycle such as

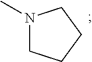

R₁₂ is selected from the group of —C₁₋₆ alkyl, —C₁₋₆ alkyl-OH; —C₁₋₆ alkyl, —C₁₋₆ substituted alkyl, —C₃₋₆ cycloalkyl, —COR₇, —COONR₁₈R₁₉, —SOR₇, and —SONR₂₀R₂₁;
R₁₃ and R₁₄ are independently selected from the group of —H, —C₁₋₆ alkyl, —C₃₋₆ cycloalkyl, —C₁₋₆ substituted alkyl, —C₁₋₆ alkyl-Q₄, —C₁₋₆ alkyl-C₃₋₆ cycloalkyl-Q₄, C₁₋₆ substituted alkyl-Q₄ and

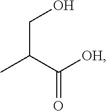

or R₁₃ and R₁₄ are taken together with the adjacent N to form a cycle selected from the group of:

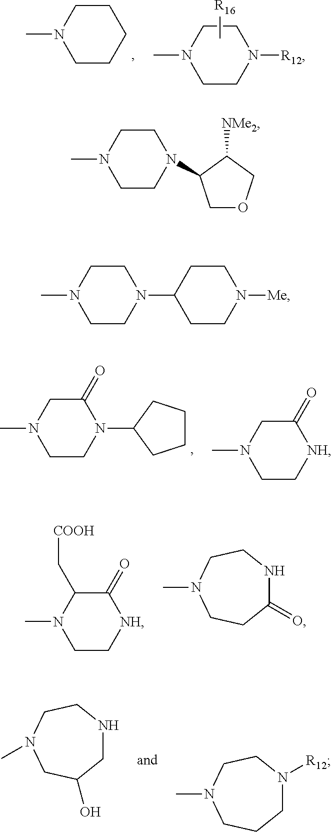

R₁₅ is selected from the group of —C₁₋₆ alkyl, —C₃₋₆ cycloalkyl, —C₁₋₆ substituted alkyl, —C₁₋₆ alkyl-Q₄, —C₁₋₆ alkyl-C₃₋₆ cycloalkyl-Q₄ and —C₁₋₆ substituted alkyl-Q₄,
Q₄ is selected from the group of heteroaryl, substituted heteroaryl, —NR₂R₂, —CONR₂R₂, —COOR₂, —OR₂, and —SO₂R₃;
R₁₆ is selected from the group of —H, —C₁₋₆ alkyl, —NR₂R₂, and —COOR₃;
R₁₇ is selected from the group of —H, —C₁₋₆ alkyl, —COOR₃, and aryl;
R₁₈ and R₁₉ are independently selected from the group of H, —C₁₋₆ alkyl, —C₁₋₆ substituted alkyl, and —C₁₋₆ cycloalkyl;
R₁₈ can also be —COOR₃;
or R₁₈ and R₁₉ are taken together with the adjacent N to form a cycle selected from the group of

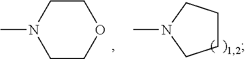

R₂₀ and R₂₁ are independently from the group of H, —C₁₋₆ alkyl, —C₁₋₆ substituted alkyl, —C₁₋₆ alkyl-Q₅, —C₁₋₆ cycloalkyl, aryl, substituted aryl, heteroaryl, and substituted heteroaryl,
Q₅ is selected from the group of halogen and SO₂R₃
R₂₄ and R₂₅ are selected from the group of —H, —C₁₋₆ alkyl, -alkylsubstituted C₁₋₆ alkyl, —SO₂R₃, —SO₂NR₂R₂ or —OH, —NR₂R₂, —NR₂SO₂R₃, —NR₂COR₃ and —NR₂CONR₂R₂; with the proviso that only one of R₂₄ and R₂₅ can be selected from the group of —OH, —NR₂R₂, —NR₂SO₂R₃, —NR₂COR₃ and —NR₂CONR₂R₂;
R₂₆ and R₂₇ are independently selected from the group of —H, —C₁₋₆ alkyl, -alkylsubstituted C₁₋₆ alkyl, —C₁₋₃ alkylaryl, C₁₋₃alkylheteroaryl, —CO₂R₂ and —SO₂R₇; with the proviso that only one of R₂₆ and R₂₇ can be selected from the group of —CO₂R₂ or —SO₂R₇;
R₂₈ and R₂₉ are independently selected from the group of —H, —C₁₋₆ alkyl, -alkylsubstituted C₁₋₆ alkyl, —C₃₋₆ cycloalkyl, —C₁₋₆ alkyl-Q₆, —COC₁₋₆ alkyl-Q₆, —COOR₃; —COCF₃;
R₂₈ can also be selected from —COOR₃ and —CONR₁₈R₁₉;
or R₂₈ and R₂₉ are taken together with the adjacent N to form a cycle selected from the group of:

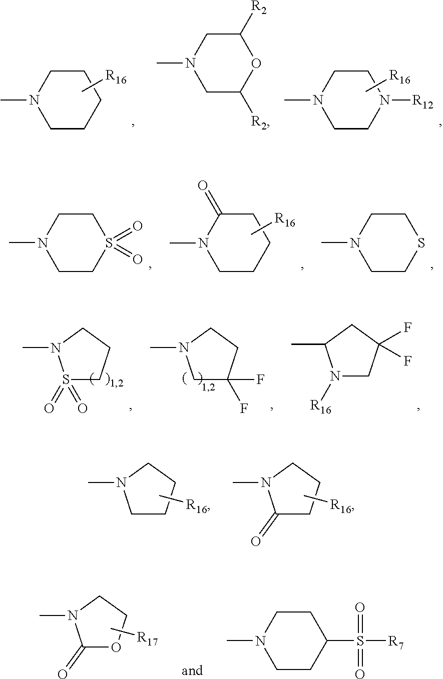

R₃₀ is selected from the group of H, —C₁₋₆ alkyl, -alkylsubstituted C₁₋₆ alkyl, —C₃₋₆ cycloalkyl, and —C₁₋₆alkyl-Q₆,
wherein Q₆ is selected from the group of H, —OR₂, —COOR₂, —COCOOR₂, —NR₃₁R₃₂;
R₃₁ and R₃₂ are independently selected from the group of —H, —C₁₋₆ alkyl, —C₁₋₆ substituted alkyl, —C₁₋₆ substituted alkyl-OR₂, and —COR_{S},
or R₃₁ and R₃₂ are taken together with the adjacent N to form a cycle selected from the group of

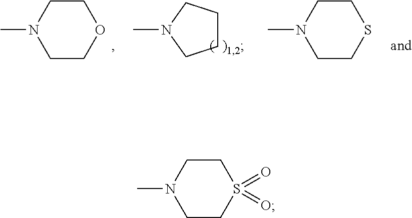

and
R₃₃ is selected from the group of —H, —C₁₋₆ alkyl, —C₁₋₆ substituted alkyl, and —C₁₋₆ substituted alkyl-Q₇,
wherein Q₇ is selected from the group of —COOR₂ and —COONR₂R₂.

In a further embodiment, there is provided a method for treating mammals infected with a virus, especially wherein said virus is HIV, comprising administering to said mammal an antiviral effective amount of a compound which is selected from the group of compounds of Formulas I and II above, and one or more pharmaceutically acceptable carriers, excipients or diluents. Optionally, the compound of Formulas I and/or II can be administered in combination with an antiviral effective amount of another—AIDS treatment agent selected from the group of: (a) an AIDS antiviral agent; (b) an anti-infective agent; (c) an immunomodulator; and (d) other HIV entry inhibitors.

Another embodiment of the present invention is a pharmaceutical composition comprising an antiviral effective amount of a compound which is selected from the group of compounds of Formulas I and II, and one or more pharmaceutically acceptable carriers, excipients, and diluents; and optionally in combination with an antiviral effective amount of another AIDS treatment agent selected from the group of: (a) an AIDS antiviral agent; (b) an anti-infective agent; (c) an immunomodulator; and (d) other HIV entry inhibitors.

In another embodiment of the invention there is provided one or more methods for making the compounds of Formulas I and II herein.

Also provided herein are intermediate compounds useful in making the compounds of Formulas I and II herein.

The present invention is directed to these, as well as other important ends, hereinafter described.

### DETAILED DESCRIPTION OF THE EMBODIMENTS

Since the compounds of the present invention may possess asymmetric centers and therefore occur as mixtures of diastereomers and enantiomers, the present disclosure includes the individual diastereoisomeric and enantiomeric forms of the compounds of Formulas I and II, in addition to the mixtures thereof.

### DEFINITIONS

Unless otherwise specifically set forth elsewhere in the application, one or more of the following terms may be used herein, and shall have the following meanings:

“H” refers to hydrogen, including its isotopes, such as deuterium.

The term “C₁₋₆ alkyl” as used herein and in the claims (unless specified otherwise) mean straight or branched chain alkyl groups such as methyl, ethyl, propyl, isopropyl, butyl, isobutyl, t-butyl, amyl, hexyl and the like.

“C₁-C₄-fluoroalkyl” refers to F-substituted C₁-C₄ alkyl wherein at least one H atom is substituted with F atom, and each H atom can be independently substituted by F atom;

“Halogen” refers to chlorine, bromine, iodine or fluorine.

An “aryl” or “Ar” group refers to an all carbon monocyclic or fused-ring polycyclic (i.e., rings which share adjacent pairs of carbon atoms) groups having a completely conjugated pi-electron system. Examples, without limitation, of aryl groups are phenyl, napthalenyl and anthracenyl. The aryl group may be substituted or unsubstituted. When substituted the substituted group(s) is preferably one or more selected from alkyl, cycloalkyl, aryl, heteroaryl, heteroalicyclic, hydroxy, alkoxy, aryloxy, heteroaryloxy, heteroalicycloxy, thiohydroxy, thioaryloxy, thioheteroaryloxy, thioheteroalicycloxy, cyano, halogen, nitro, carbonyl, O-carbamyl, N-carbamyl, C-amido, N-amido, C-carboxy, O-carboxy, sulfinyl, sulfonyl, sulfonamido, trihalomethyl, ureido, amino and —NR^{x}R^{y}, wherein R^{x} and R^{y} are independently selected from the group of hydrogen, alkyl, cycloalkyl, aryl, carbonyl, C-carboxy, sulfonyl, trihalomethyl, and, combined, a five- or six-member heteroalicyclic ring.

As used herein, a “heteroaryl” group refers to a monocyclic or fused ring (i.e., rings which share an adjacent pair of atoms) group having in the ring(s) one or more atoms selected from the group of nitrogen, oxygen and sulfur and, in addition, having a completely conjugated pi-electron system. Unless otherwise indicated, the heteroaryl group may be attached at either a carbon or nitrogen atom within the heteroaryl group. It should be noted that the term heteroaryl is intended to encompass an N-oxide of the parent heteroaryl if such an N-oxide is chemically feasible as is known in the art. Examples, without limitation, of heteroaryl groups are furyl, thienyl, benzothienyl, thiazolyl, imidazolyl, oxazolyl, oxadiazolyl, thiadiazolyl, benzothiazolyl, triazolyl, tetrazolyl, isoxazolyl, isothiazolyl, pyrrolyl, pyranyl, tetrahydropyranyl, pyrazolyl, pyridyl, pyrimidinyl, quinolinyl, isoquinolinyl, purinyl, carbazolyl, benzoxazolyl, benzimidazolyl, indolyl, isoindolyl, pyrazinyl. diazinyl, pyrazine, triazinyl, tetrazinyl, and tetrazolyl. When substituted the substituted group(s) is preferably one or more selected from alkyl, cycloalkyl, aryl, heteroaryl, heteroalicyclic, hydroxy, alkoxy, aryloxy, heteroaryloxy, heteroalicycloxy, thioalkoxy, thiohydroxy, thioaryloxy, thioheteroaryloxy, thioheteroalicycloxy, cyano, halogen, nitro, carbonyl, O-carbamyl, N-carbamyl, C-amido, N-amido, C-carboxy, O-carboxy, sulfinyl, sulfonyl, sulfonamido, trihalomethyl, ureido, amino, and —NR^{x}R^{y}, wherein R^{x} and R^{y} are as defined above.

As used herein, a “heteroalicyclic” group refers to a monocyclic or fused ring group having in the ring(s) one or more atoms selected from the group of nitrogen, oxygen and sulfur. Rings are selected from those which provide stable arrangements of bonds and are not intended to encompass systems which would not exist. The rings may also have one or more double bonds. However, the rings do not have a completely conjugated pi-electron system. Examples, without limitation, of heteroalicyclic groups are azetidinyl, piperidyl, piperazinyl, imidazolinyl, thiazolidinyl, 3-pyrrolidin-1-yl, morpholinyl, thiomorpholinyl and tetrahydropyranyl. When substituted the substituted group(s) is preferably one or more selected from alkyl, cycloalkyl, aryl, heteroaryl, heteroalicyclic, hydroxy, alkoxy, aryloxy, heteroaryloxy, heteroalicycloxy, thiohydroxy, thioalkoxy, thioaryloxy, thioheteroaryloxy, thioheteroalicycloxy, cyano, halogen, nitro, carbonyl, thiocarbonyl, O-carbamyl, N-carbamyl, O-thiocarbamyl, N-thiocarbamyl, C-amido, C-thioamido, N-amido, C-carboxy, O-carboxy, sulfinyl, sulfonyl, sulfonamido, trihalomethanesulfonamido, trihalomethanesulfonyl, silyl, guanyl, guanidino, ureido, phosphonyl, amino and —NR^{x}R^{y}, wherein R^{x} and R^{y} are as defined above.

An “alkyl” group refers to a saturated aliphatic hydrocarbon including straight chain and branched chain groups. Preferably, the alkyl group has 1 to 20 carbon atoms (whenever a numerical range; e.g., “1-20”, is stated herein, it means that the group, in this case the alkyl group may contain 1 carbon atom, 2 carbon atoms, 3 carbon atoms, etc. up to and including 20 carbon atoms). More preferably, it is a medium size alkyl having 1 to 10 carbon atoms. Most preferably, it is a lower alkyl having 1 to 4 carbon atoms. The alkyl group may be substituted or unsubstituted. When substituted, the substituent group(s) is preferably one or more individually selected from trihaloalkyl, cycloalkyl, aryl, heteroaryl, heteroalicyclic, hydroxy, alkoxy, aryloxy, heteroaryloxy, heteroalicycloxy, thiohydroxy, thioalkoxy, thioaryloxy, thioheteroaryloxy, thioheteroalicycloxy, cyano, halo, nitro, carbonyl, thiocarbonyl, O-carbamyl, N-carbamyl, O-thiocarbamyl, N-thiocarbamyl, C-amido, C-thioamido, N-amido, C-carboxy, O-carboxy, sulfinyl, sulfonyl, sulfonamido, trihalomethanesulfonamido, trihalomethanesulfonyl, and combined, a five- or six-member heteroalicyclic ring.

A “cycloalkyl” group refers to an all-carbon monocyclic or fused ring (i.e., rings which share and adjacent pair of carbon atoms) group wherein one or more rings does not have a completely conjugated pi-electron system. Examples, without limitation, of cycloalkyl groups are cyclopropane, cyclobutane, cyclopentane, cyclopentene, cyclohexane, cyclohexene, cycloheptane, cycloheptene and adamantane. A cycloalkyl group may be substituted or unsubstituted. When substituted, the substituent group(s) is preferably one or more individually selected from alkyl, aryl, heteroaryl, heteroalicyclic, hydroxy, alkoxy, aryloxy, heteroaryloxy, heteroalicycloxy, thiohydroxy, thioalkoxy, thioaryloxy, thioheteroaryloxy, thioheteroalicycloxy, cyano, halo, nitro, carbonyl, thiocarbonyl, O-carbamyl, N-carbamyl, O-thiocarbamyl, N-thiocarbamyl, C-amido, C-thioamido, N-amido, C-carboxy, O-carboxy, sulfinyl, sulfonyl, sulfonamido, trihalo-methanesulfonamido, trihalomethanesulfonyl, silyl, amidino, guanidino, ureido, phosphonyl, amino and —NR^{x}R^{y} with R^{x} and R^{y} as defined above.

An “alkenyl” group refers to an alkyl group, as defined herein, having at least two carbon atoms and at least one carbon-carbon double bond.

An “alkynyl” group refers to an alkyl group, as defined herein, having at least two carbon atoms and at least one carbon-carbon triple bond.

A “hydroxy” group refers to an —OH group.

An “alkoxy” group refers to both an —O-alkyl and an —O-cycloalkyl group as defined herein.

An “aryloxy” group refers to both an —O-aryl and an —O-heteroaryl group, as defined herein.

A “heteroaryloxy” group refers to a heteroaryl-O— group with heteroaryl as defined herein.

A “heteroalicycloxy” group refers to a heteroalicyclic-O— group with heteroalicyclic as defined herein.

A “thiohydroxy” group refers to an —SH group.

A “thioalkoxy” group refers to both an S-alkyl and an —S-cycloalkyl group, as defined herein.

A “thioaryloxy” group refers to both an —S-aryl and an —S-heteroaryl group, as defined herein.

A “thioheteroaryloxy” group refers to a heteroaryl-S— group with heteroaryl as defined herein.

A “thioheteroalicycloxy” group refers to a heteroalicyclic-S— group with heteroalicyclic as defined herein.

A “carbonyl” group refers to a —C(═O)—R″ group, where R″ is selected from the group of hydrogen, alkyl, alkenyl, alkynyl, cycloalkyl, aryl, heteroaryl (bonded through a ring carbon) and heteroalicyclic (bonded through a ring carbon), as each is defined herein.

An “aldehyde” group refers to a carbonyl group where R″ is hydrogen.

A “thiocarbonyl” group refers to a —C(═S)—R″ group, with R″ as defined herein.

A “Keto” group refers to a —CC(═O)C— group wherein the carbon on either or both sides of the C═O may be alkyl, cycloalkyl, aryl or a carbon of a heteroaryl or heteroalicyclic group.

A “trihalomethanecarbonyl” group refers to a Z₃CC(═O)— group with said Z being a halogen.

A “C-carboxy” group refers to a —C(═O)O—R″ groups, with R″ as defined herein.

An “O-carboxy” group refers to a R″C(—O)O-group, with R″ as defined herein.

A “carboxylic acid” group refers to a C-carboxy group in which R″ is hydrogen.

A “trihalomethyl” group refers to a —CZ₃, group wherein Z is a halogen group as defined herein.

A “trihalomethanesulfonyl” group refers to an Z₃CS(═O)₂— groups with Z as defined above.

A “trihalomethanesulfonamido” group refers to a Z₃CS(═O)₂NR^{x}— group with Z as defined above and R^{x} being H or (C₁₋₆)alkyl.

A “sulfinyl” group refers to a —S(═O)—R″ group, with R″ being (C₁₋₆)alkyl.

A “sulfonyl” group refers to a —S(═O)₂R″ group with R″ being (C₁₋₆)alkyl.

A “S-sulfonamido” group refers to a —S(═O)₂NR^{X}R^{Y}, with R^{X} and R^{Y} independently being H or (C₁₋₆)alkyl.

A “N-Sulfonamido” group refers to a R″S(═O)₂NR^{x}— group, with Rx being H or (C₁₋₆)alkyl.

A “O-carbamyl” group refers to a —OC(═O)NR^{x}R^{y} group, with R^{X} and R^{Y} independently being H or (C₁₋₆)alkyl.

A “N-carbamyl” group refers to a R^{x}OC(═O)NR^{y} group, with R^{x} and R^{y} independently being H or (C₁₋₆)alkyl.

A “O-thiocarbamyl” group refers to a —OC(═S)NR^{x}R^{y} group, with R^{x} and R^{y} independently being H or (C₁₋₆)alkyl.

A “N-thiocarbamyl” group refers to a R^{x}OC(═S)NR^{y}— group, with R^{x} and R^{y} independently being H or (C₁₋₆)alkyl.

An “amino” group refers to an —NH₂ group.

A “C-amido” group refers to a —C(═O)NR^{x}R^{y} group, with R^{x} and R^{y} independently being H or (C₁₋₆)alkyl.

A “C-thioamido” group refers to a —C(═S)NR^{x}R^{y} group, with R^{x} and R^{y} independently being H or (C₁₋₆)alkyl.

A “N-amido” group refers to a —R^{x}C(═O)NR^{y}— group, with R^{x} and R^{y} independently being H or (C₁₋₆)alkyl.

An “ureido” group refers to a —NR^{x}C(═O)NR^{y}R^{y2} group, with R^{x}, R^{y}, and R^{y}R^{y2} independently being H or (C₁₋₆)alkyl.

A “guanidino” group refers to a —R^{x}NC(═N)NR^{y}R^{y2} group, with R^{x}, R^{y}, and R^{y}R^{y2} independently being H or (C₁₋₆)alkyl.

A “amidino” group refers to a R^{x}R^{y}NC(═N)— group, with R^{x} and R^{y} independently being H or (C₁₋₆)alkyl.

A “cyano” group refers to a —CN group.

A “silyl” group refers to a —Si(R″)₃, with R″ being (C₁₋₆)alkyl or phenyl.

A “phosphonyl” group refers to a P(═O)(OR^{x})₂ with R^{x} being (C₁₋₆)alkyl.

A “hydrazino” group refers to a —NR^{x}NR^{y}R^{y2} group, with Rx, R^{y}, and R^{y}R^{y2} independently being H or (C₁₋₆)alkyl.

A “4, 5, or 6 membered ring cyclic N-lactam” group refers to

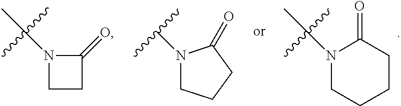 [SMILES: *N1CCC1=O ; *N1CCCC1=O ; "or" ; *N1CCCCC1=O ; "."]

Any two adjacent R groups may combine to form an additional aryl, cycloalkyl, heteroaryl or heterocyclic ring fused to the ring initially bearing those R groups.

It is known in the art that nitrogen atoms in heteroaryl systems can be “participating in a heteroaryl ring double bond”, and this refers to the form of double bonds in the two tautomeric structures which comprise five-member ring heteroaryl groups. This dictates whether nitrogens can be substituted as well understood by chemists in the art. The disclosure and claims of the present disclosure are based on the known general principles of chemical bonding. It is understood that the claims do not encompass structures known to be unstable or not able to exist based on the literature.

Pharmaceutically acceptable salts and prodrugs of compounds disclosed herein are within the scope of the invention. The term “pharmaceutically acceptable salt” as used herein and in the claims is intended to include nontoxic base addition salts. Suitable salts include those derived from organic and inorganic acids such as, without limitation, hydrochloric acid, hydrobromic acid, phosphoric acid, sulfuric acid, methanesulfonic acid, acetic acid, tartaric acid, lactic acid, sulfinic acid, citric acid, maleic acid, fumaric acid, sorbic acid, aconitic acid, salicylic acid, phthalic acid, and the like. The term “pharmaceutically acceptable salt” as used herein is also intended to include salts of acidic groups, such as a carboxylate, with such counterions as ammonium, alkali metal salts, particularly sodium or potassium, alkaline earth metal salts, particularly calcium or magnesium, and salts with suitable organic bases such as lower alkylamines (methylamine, ethylamine, cyclohexylamine, and the like) or with substituted lower alkylamines (e.g. hydroxyl-substituted alkylamines such as diethanolamine, triethanolamine or tris(hydroxymethyl)-aminomethane), or with bases such as piperidine or morpholine.

As stated above, the compounds of the invention also include “prodrugs”. The term “prodrug” as used herein encompasses both the term “prodrug esters” and the term “prodrug ethers”.

The term “C-19” and “C-3” refer to certain positions of a triterpene core as numbered in accordance with IUPAC rules (positions depicted below with respect to an illustrative triterpene: betulin):

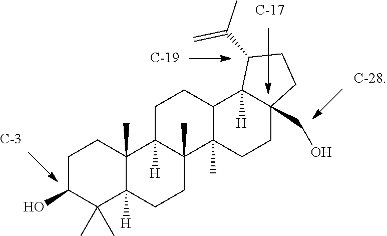

As set forth above, the invention is directed to a compound, including pharmaceutically acceptable salts thereof, which is selected from the group of:

a compound of formula I

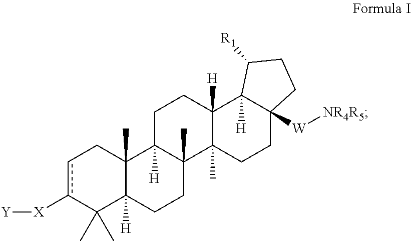

and
a compound of formula II

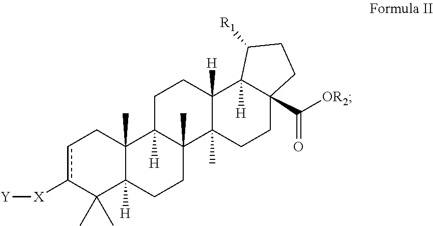

wherein X is selected from the group of phenyl, heteroaryl ring, C₄₋₈ cycloalkyl, C₄₋₈ cycloalkenyl, C₄₋₉ spirocycloalkyl, C₄₋₉ spirocycloalkenyl, C₄₋₈ oxacycloalkyl, C₄₋₈ dioxacycloalkyl, C₆₋₈ oxacycloalkenyl, C₆₋₈ dioxacycloalkenyl, C₆ cyclodialkenyl, C₆ oxacyclodialkenyl, C₆₋₉ oxaspirocycloalkyl and C₆₋₉ oxaspirocycloalkenyl ring;
and further wherein X is substituted with A, wherein A is at least one member selected from the group of —H, -halo, -hydroxyl, —C₁₋₆ alkyl, —C₁₋₆ alkoxy, —C₁₋₆ alkyl-Q₁, -alkylsubstituted C₁₋₆ alkyl-Qi, —CN, —CF₂Q₁, —NR₂R₂, —COOR₂ and —CONR₂R₂,
wherein Q₁ is selected from the group of aryl, heteroaryl, substituted heteroaryl, —OR₂, —COOR₃, —NR₂R₂, —SO₂R₇, —CONHSO₂R₃, and —CONHSO₂NR₂R₂;
Y is selected from the group of —COOR₂, —C(O)NR₂SO₂R₃, —C(O)NHSO₂NR₂R₂, —NR₂SO₂R₂, —SO₂NR₂R₂, —C₃₋₆ cycloalkyl-COOR₂, —C₂₋₆ alkenyl-COOR₂, —C₂₋₆ alkynyl-COOR₂, —C₁₋₆ alkyl-COOR₂, -alkylsubstituted C₁₋₆ alkyl, —COOR₂, CF₂—COOR₂, —NHC(O)(CH₂)ₙ—COOR₂, —SO₂NR₂C(O)R₂, _tetrazole, and —CONHOH, wherein n=1-6;
R₁ is selected from the group of:

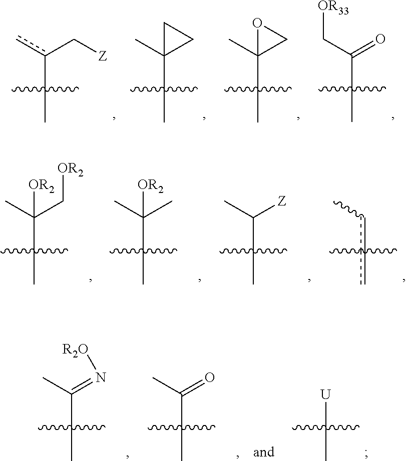

W is absent, or is —CH₂ or —CO;
Z is selected from the group of —NR₂₈R₂₉, —OR₃₀, —COOR₂, —CONR₁₈R₁₉, F, Cl, Br, and I;
U is selected from the group of —NR₂₈R₂₉, —OR₃₀, —COOR₂, —CONR₁₈R₁₉, F, Cl, Br, I, aryl and heteroaryl;
R₂ is selected from the group of —H, benzyl, —C₁₋₆ alkyl, -alkylsubstituted C₁₋₆ alkyl and -arylsubstituted C₁₋₆ alkyl;
R₃ is benzyl, —C₁₋₆ alkyl or -alkylsubstituted C₁₋₆ alkyl;
R₄ is selected from the group of —H, —C₁₋₆ alkyl, —C₁₋₆ alkyl-C(OR₃)₂—C₃₋₆ cycloalkyl, —C₁₋₆ substituted alkyl, —C₁₋₆ alkyl-C₃₋₆ cycloalkyl, —C₁₋₆ alkyl-Q₂, —C₁₋₆ alkyl-C₃₋₆ cycloalkyl-Q₂, aryl, heteroaryl, substituted heteroaryl, —COR₆, —COCOR₆, —SO₂R₇, —SO₂NR₂R₂,

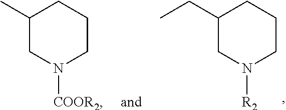

wherein Q₂ is selected from the group of heteroaryl, substituted heteroaryl, F, Cl, Br, I, —CF₃, —OR₂, —COOR₂, —NR₈R₉, —CONR₁₀R₁₁ and —SO₂R₇;
R₅ is selected from the group of —H, —C₁₋₆ alkyl, —C₃₋₆ cycloalkyl, —C₁₋₆ alkylsubstituted alkyl, —C₁₋₆ alkyl-NR₈R₉, —COR_{E}, —COCOR₆, —SO₂R₂ and —SO₂NR₂R₂;
with the proviso that R₄ or R₅ cannot be COR₆ or COCOR₆ when W is CO;
with the further proviso that only one of R₄ or R₅ can be selected from the group of —COR₆, —COCOR₆, —SO₂R₂ and —SO₂NR₂R₂;
or when W is absent or is CH₂, then R₄ and R₅ can be taken together with the adjacent N to form

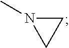

R₆ is selected from the group of —C₁₋₆ alkyl, —C₁₋₆ alkyl-substitutedalkyl, —C₃₋₆ cycloalkyl, —C₃₋₆ substitutedcycloalkyl-Q₃, —C₁₋₆ alkyl-Q₃, —C₁₋₆ alkyl-substitutedalkyl-Q₃, —C₃₋₆ cycloalkyl-Q₃, aryl-Q₃, —NR₁₃R₁₄, and —OR₁₅;
wherein Q₃ is selected from the group of aryl, heteroaryl, substituted heteroaryl, —OR₂, —COOR₂, —NR₈R₉, SO₂R₇, —CONHSO₂R₃, and —CONHSO₂NR₂R₂;
R₇ is selected from the group of —C₁₋₆ alkyl, —C₁₋₆ substituted alkyl, —C₃₋₆ cycloalkyl, —CF₃, aryl, and heteroaryl;
R₈ and R₉ are independently selected from the group of —H, —C₁₋₆ alkyl, —C₁₋₆ substituted alkyl, aryl, heteroaryl, substituted aryl, substituted heteroaryl, —C₁₋₆ alkyl-Q₂, and —COOR₃:
R₈ can also be —COOR₃;
R₈ and R₉ can also be independently selected from the group of

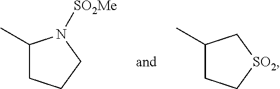

or R₈ and R₉ are taken together with the adjacent N to form a cycle selected from the group of:

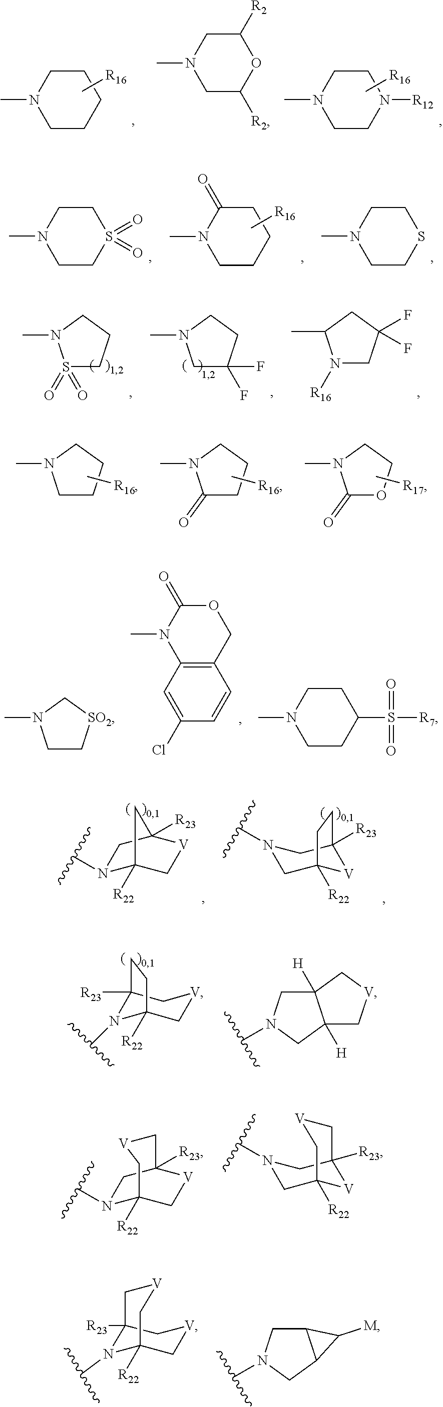

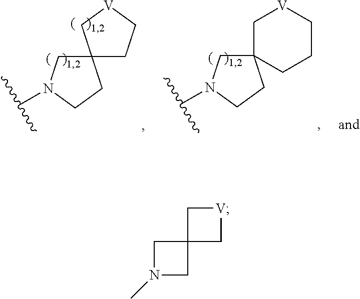

V is selected from the group of —CR₂₄R₂₅, —SO₂, —O and —NR₁₂;
M is selected from the group of —CHR₂₄R₂₅, —NR₂₆R₂₇, —SO₂R₇, —SO₂NR₃R₃ and —OH;
R₁₀ and R₁₁ are independently selected from the group of —H, —C₁₋₆ alkyl, —C₁₋₆ substituted alkyl and —C₃₋₆ cycloalkyl,
or R₁₀ and R₁₁ are taken together with the adjacent N to form a cycle such as

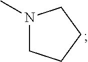

R₁₂ is selected from the group of —C₁₋₆ alkyl, —C₁₋₆ alkyl-OH; —C₁₋₆ alkyl, —C₁₋₆ substituted alkyl, —C₃₋₆ cycloalkyl, —COR₇, —COONR₁₈R₁₉, —SOR_{T}, and —SONR₂₀R₂₁;
R₁₃ and R₁₄ are independently selected from the group of —H, —C₁₋₆ alkyl, —C₃₋₆ cycloalkyl, —C₁₋₆ substituted alkyl, —C₁₋₆ alkyl-Q₄, —C₁₋₆ alkyl-C₃₋₆ cycloalkyl-Q₄, C₁₋₆ substituted alkyl-Q₄ and

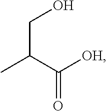

or R₁₃ and R₁₄ are taken together with the adjacent N to form a cycle selected from the group of:

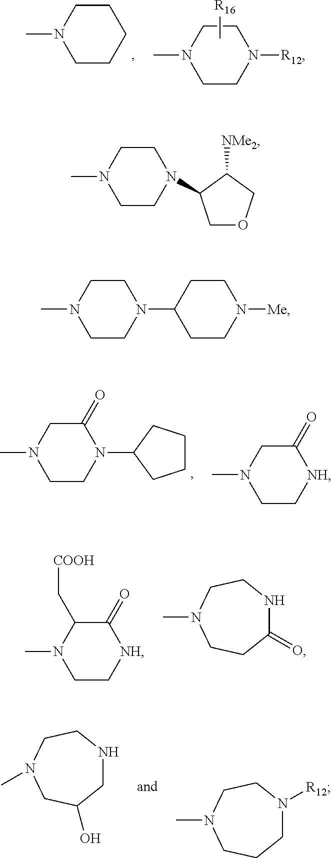

R₁₅ is selected from the group of —C₁₋₆ alkyl, —C₃₋₆ cycloalkyl, —C₁₋₆ substituted alkyl, —C₁₋₆ alkyl-Q₄, —C₁₋₆ alkyl-C₃₋₆ cycloalkyl-Q₄ and —C₁₋₆ substituted alkyl-Q₄,
Q₄ is selected from the group of heteroaryl, substituted heteroaryl, —NR₂R₂, —CONR₂R₂, —COOR₂, —OR₂, and —SO₂R₃;
R₁₆ is selected from the group of —H, —C₁₋₆ alkyl, —NR₂R₂, and —COOR₃;
R₁₇ is selected from the group of —H, —C₁₋₆ alkyl, —COOR₃, and aryl;
R₁₈ and R₁₉ are independently selected from the group of H, —C₁₋₆ alkyl, —C₁₋₆ substituted alkyl, and —C₁₋₆ cycloalkyl;
R₁₈ can also be —COOR₃;
or R₁₈ and R₁₉ are taken together with the adjacent N to form a cycle selected from the group of

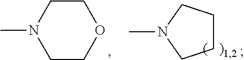

R₂₀ and R₂₁ are independently from the group of H, —C₁₋₆ alkyl, —C₁₋₆ substituted alkyl, —C₁₋₆ alkyl-Q₅, —C₁₋₆ cycloalkyl, aryl, substituted aryl, heteroaryl, and substituted heteroaryl,
Q₅ is selected from the group of halogen and SO₂R₃
R₂₄ and R₂₅ are independently selected from the group of —H, —C₁₋₆ alkyl, -alkylsubstituted C₁₋₆ alkyl, —SO₂R₃, —SO₂NR₂R₂ or —OH, —NR₂R₂, —NR₂SO₂R₃, —NR₂COR₃ and —NR₂CONR₂R₂;
with the proviso that only one of R₂₄ and R₂₅ can be selected from the group of —OH, —NR₂R₂, —NR₂SO₂R₃, —NR₂COR₃ and —NR₂CONR₂R₂;
R₂₆ and R₂₇ are independently selected from the group of —H, —C₁₋₆ alkyl, -alkylsubstituted C₁₋₆ alkyl, —C₁₋₃ alkylaryl, C₁₋₃alkylheteroaryl, —CO₂R₂ and —SO₂R₇;
with the proviso that only one of R₂₆ and R₂₇ can be selected from the group of —CO₂R₂ or —SO₂R₇;
R₂₈ and R₂₉ are independently selected from the group of H, —C₁₋₆ alkyl, -alkylsubstituted C₁₋₆ alkyl, —C₃₋₆ cycloalkyl, —C₁₋₆ alkyl-Q₆, —COC₁₋₆ alkyl-Q₆, —COOR₃; —COCF₃;
R₂₈ can also be selected from —COOR₃ and —CONR₁₈R₁₉;
or R₂₈ and R₂₉ are taken together with the adjacent N to form a cycle selected from the group of:

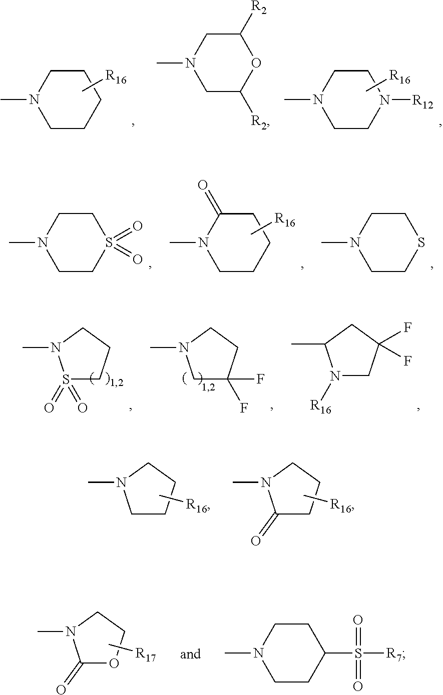

R₃₀ is selected from the group of H, —C₁₋₆ alkyl, -alkylsubstituted C₁₋₆ alkyl, —C₃₋₆ cycloalkyl, and —C₁₋₆alkyl-Q₆,
wherein Q₆ is selected from the group of H, —OR₂, —COOR₂, —COCOOR₂, and —NR₃₁R₃₂;
R₃₁ and R₃₂ are independently selected from the group of —H, —C₁₋₆ alkyl, —C₁₋₆ substituted alkyl, —C₁₋₆ substituted alkyl-OR₂, and —COR₃, or R₃₁ and R₃₂ are taken together with the adjacent N to form a cycle selected from the group of

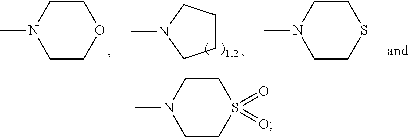

and
R₃₃ is selected from the group of —H, —C₁₋₆ alkyl, —C₁₋₆ substituted alkyl, and —C₁₋₆ substituted alkyl-Q₇,
wherein Q₇ is selected from the group of —COOR₂ and —COONR₂R₂.

In particular, compounds of Formula I and II are preferred wherein X is phenyl.

Also preferred are compounds wherein A is —H or halo, especially —F.

Further preferred are compounds wherein Y is —COOR₂. It is also preferred that R₂ is —H.

Also preferred are compounds, including pharmaceutically acceptable salts thereof, which are selected from the group of

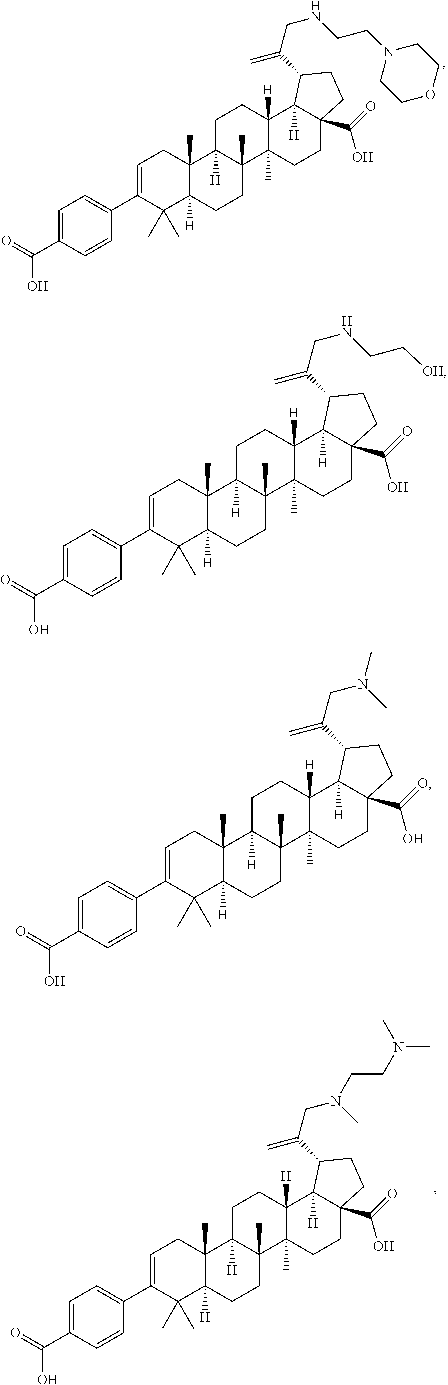

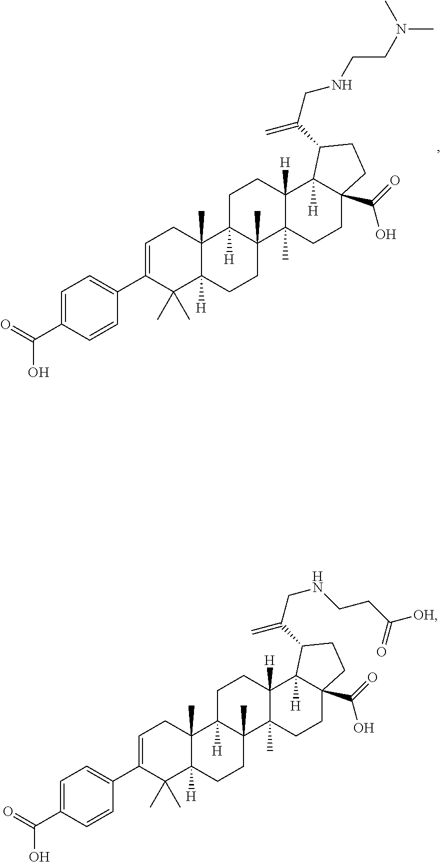

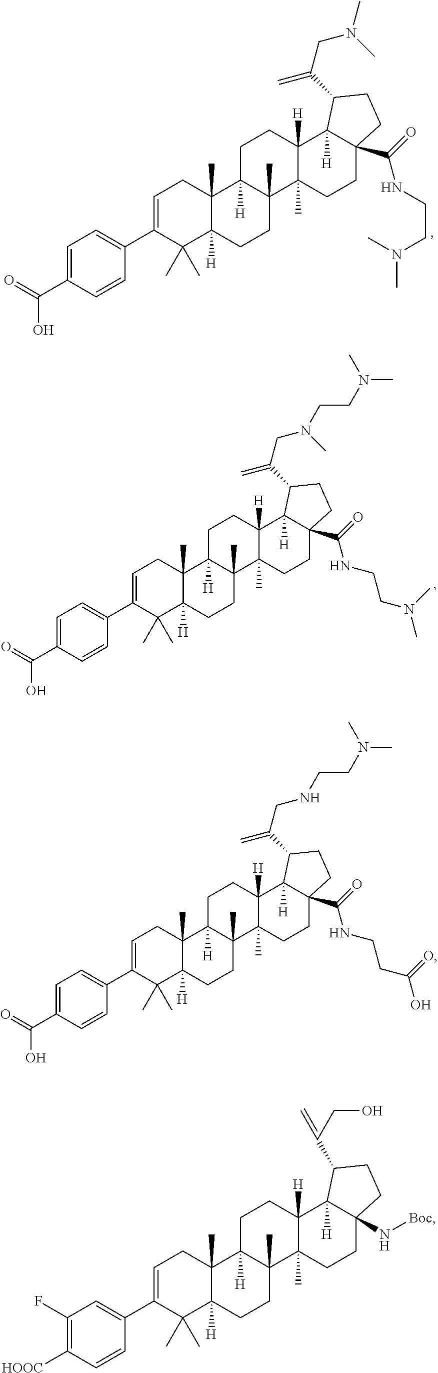

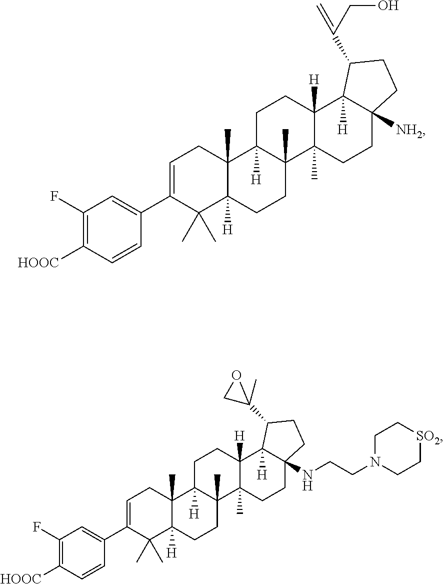

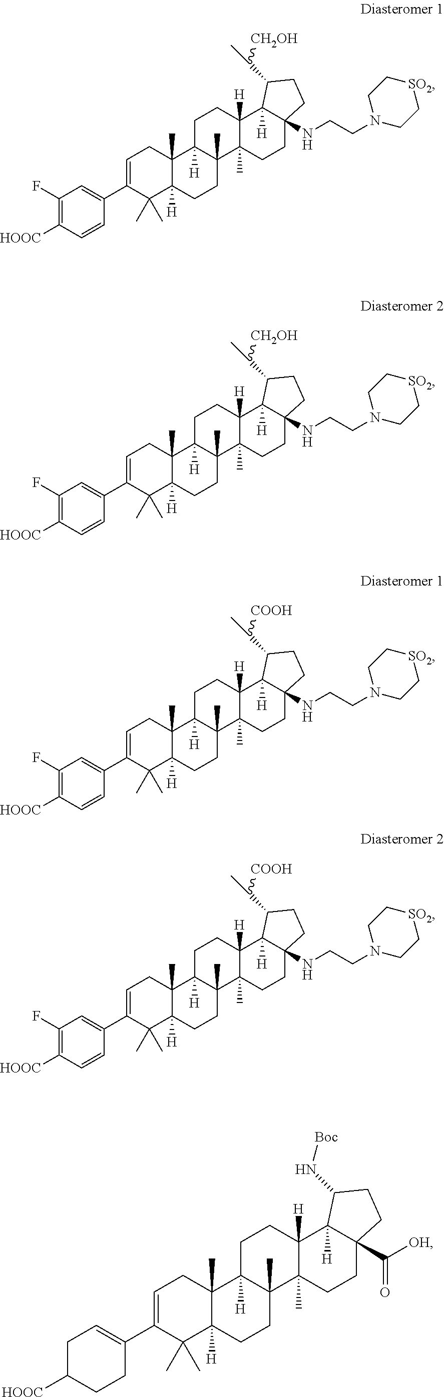

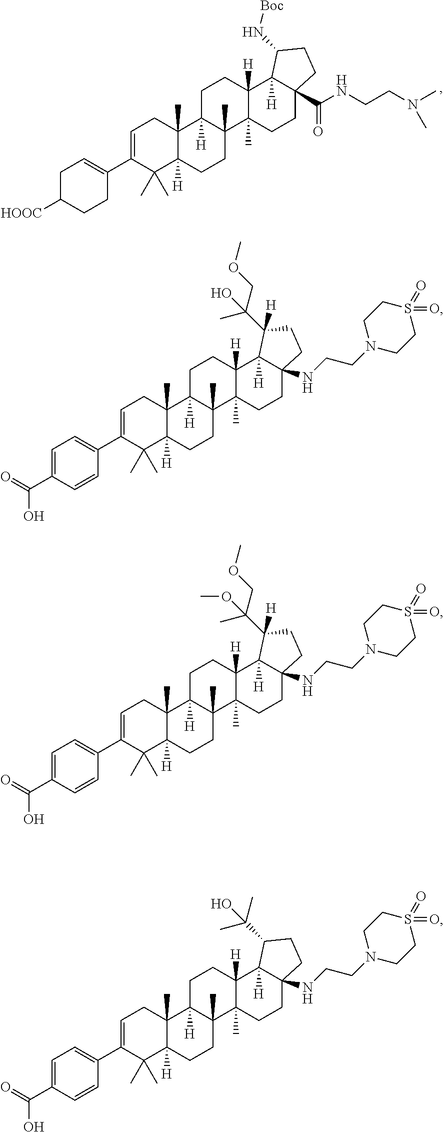

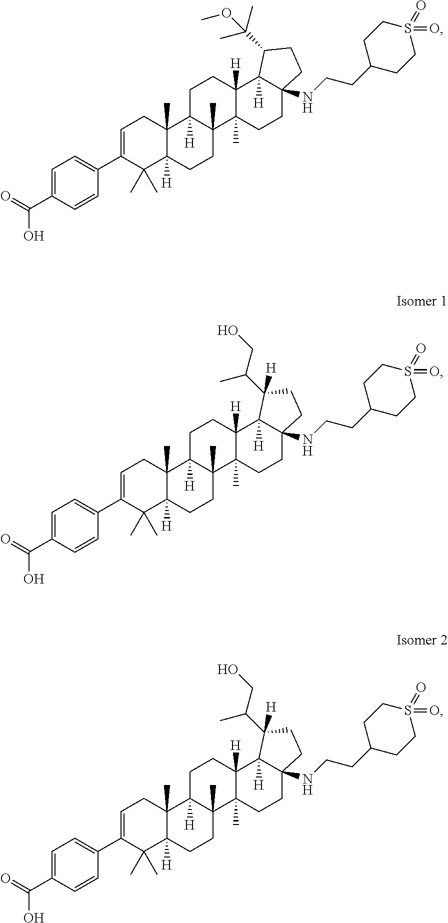

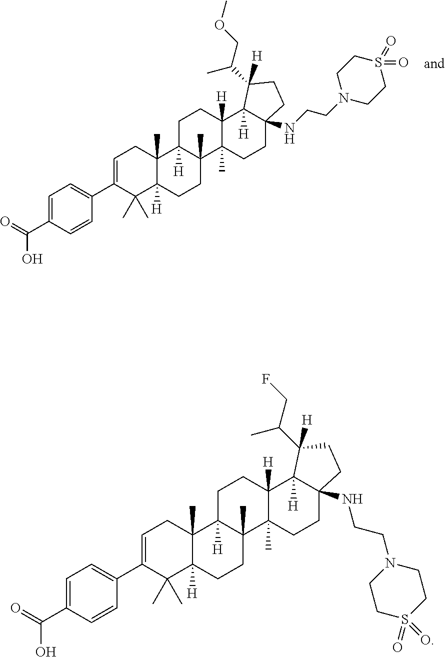

The compounds of the present invention, according to all the various embodiments described above, may be administered orally, parenterally (including subcutaneous injections, intravenous, intramuscular, intrasternal injection or infusion techniques), by inhalation spray, or rectally, and by other means, in dosage unit formulations containing non-toxic pharmaceutically acceptable carriers, excipients and diluents available to the skilled artisan. One or more adjuvants may also be included.

Thus, in accordance with the present invention, there is further provided a method of treatment, and a pharmaceutical composition, for treating viral infections such as HIV infection and AIDS. The treatment involves administering to a patient in need of such treatment a pharmaceutical composition which contains an antiviral effective amount of one or more of the compounds of Formulas I and II, together with one or more pharmaceutically acceptable carriers, excipients or diluents. As used herein, the term “antiviral effective amount” means the total amount of each active component of the composition and method that is sufficient to show a meaningful patient benefit, i.e., inhibiting, ameliorating, or healing of acute conditions characterized by inhibition of the HIV infection. When applied to an individual active ingredient, administered alone, the term refers to that ingredient alone. When applied to a combination, the term refers to combined amounts of the active ingredients that result in the therapeutic effect, whether administered in combination, serially or simultaneously. The terms “treat, treating, treatment” as used herein and in the claims means preventing, ameliorating or healing diseases associated with HIV infection.

The pharmaceutical compositions of the invention may be in the form of orally administrable suspensions or tablets; as well as nasal sprays, sterile injectable preparations, for example, as sterile injectable aqueous or oleaginous suspensions or suppositories. Pharmaceutically acceptable carriers, excipients or diluents may be utilized in the pharmaceutical compositions, and are those utilized in the art of pharmaceutical preparations.

When administered orally as a suspension, these compositions are prepared according to techniques typically known in the art of pharmaceutical formulation and may contain microcrystalline cellulose for imparting bulk, alginic acid or sodium alginate as a suspending agent, methylcellulose as a viscosity enhancer, and sweeteners/flavoring agents known in the art. As immediate release tablets, these compositions may contain microcrystalline cellulose, dicalcium phosphate, starch, magnesium stearate and lactose and/or other excipients, binders, extenders, disintegrants, diluents, and lubricants known in the art.

The injectable solutions or suspensions may be formulated according to known art, using suitable non-toxic, parenterally acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

The compounds herein set forth can be administered orally to humans in a dosage range of about 1 to 100 mg/kg body weight in divided doses, usually over an extended period, such as days, weeks, months, or even years. One preferred dosage range is about 1 to 10 mg/kg body weight orally in divided doses. Another preferred dosage range is about 1 to 20 mg/kg body weight in divided doses. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Also contemplated herein are combinations of the compounds of Formulas I and II herein set forth, together with one or more other agents useful in the treatment of AIDS. For example, the compounds of this disclosure may be effectively administered, whether at periods of pre-exposure and/or post-exposure, in combination with effective amounts of the AIDS antivirals, immunomodulators, antiinfectives, or vaccines, such as those in the following non-limiting table:

| | | |
|---|---|---|
| Drug Name | Manufacturer | Indication |

ANTIVIRALS

| | | |
|---|---|---|
| 097 | Hoechst/Bayer | HIV infection, |
| | | AIDS, ARC |
| | | (non-nucleoside |
| | | reverse transcriptase |
| | | (RT) |
| | | inhibitor) |
| Amprenavir | Glaxo Wellcome | HIV infection, |
| 141 W94 | | AIDS, ARC |
| GW 141 | | (protease inhibitor) |
| Abacavir (1592U89) | Glaxo Wellcome | HIV infection, |
| GW 1592 | | AIDS, ARC |
| Beta-fluoro-ddA | Nat'l Cancer Institute | AIDS-associated |
| | | diseases |
| BMS-234475 | Bristol-Myers Squibb/ | HIV infection, |
| (CGP-61755) | Novartis | AIDS, ARC |
| | | (protease inhibitor) |
| CI-1012 | Warner-Lambert | HIV-1 infection |
| Cidofovir | Gilead Science | CMV retinitis, |
| | | herpes, papillomavirus |
| Curdlan sulfate | AJI Pharma USA | HIV infection |
| Cytomegalovirus | MedImmune | CMV retinitis |
| Immune globin | | |
| Cytovene | Syntex | Sight threatening |
| Ganciclovir | | CMV |
| | | peripheral CMV |
| | | retinitis |
| Darunavir | Tibotec-J & J | HIV infection, AIDS, ARC |
| | | (protease inhibitor) |
| Delaviridine | Pharmacia-Upjohn | HIV infection, |
| | | AIDS, ARC |
| | | (RT inhibitor) |
| Dextran Sulfate | Ueno Fine Chem. | AIDS, ARC, HIV |
| | Ind. Ltd. (Osaka, | positive |
| | Japan) | asymptomatic |
| ddC | Hoffman-La Roche | HIV infection, AIDS, |
| Dideoxycytidine | | ARC |
| ddI | Bristol-Myers Squibb | HIV infection, AIDS, |
| Dideoxyinosine | | ARC; combination |
| | | with AZT/d4T |
| DMP-450 | AVID | HIV infection, |
| | (Camden, NJ) | AIDS, ARC |
| | | (protease inhibitor) |
| Efavirenz | Bristol Myers Squibb | HIV infection, |
| (DMP 266, SUSTIVA ®) | | AIDS, ARC |
| (−)6-Chloro-4-(S)- | | (non-nucleoside RT |
| cyclopropylethynyl- | | inhibitor) |
| 4(S)-trifluoro- | | |
| methyl-1,4-dihydro- | | |
| 2H-3,1-benzoxazin- | | |
| 2-one, STOCRINE | | |
| EL10 | Elan Corp, PLC | HIV infection |
| | (Gainesville, GA) | |
| Etravirine | Tibotec/J & J | HIV infection, AIDS, ARC |
| | | (non-nucleoside |
| | | reverse transcriptase |
| | | inhibitor) |
| Famciclovir | Smith Kline | herpes zoster, |
| | | herpes simplex |
| GS 840 | Gilead | HIV infection, |
| | | AIDS, ARC |
| | | (reverse transcriptase |
| | | inhibitor) |
| HBY097 | Hoechst Marion | HIV infection, |
| | Roussel | AIDS, ARC |
| | | (non-nucleoside |
| | | reverse transcriptase |
| | | inhibitor) |
| Hypericin | VIMRx Pharm. | HIV infection, AIDS, |
| | | ARC |
| Recombinant Human | Triton Biosciences | AIDS, Kaposi's |
| Interferon Beta | (Almeda, CA) | sarcoma, ARC |
| Interferon alfa-n3 | Interferon Sciences | ARC, AIDS |
| Indinavir | Merck | HIV infection, AIDS, |
| | | ARC, asymptomatic |
| | | HIV positive, also in |
| | | combination with |
| | | AZT/ddI/ddC |
| ISIS 2922 | ISIS Pharmaceuticals | CMV retinitis |
| KNI-272 | Nat'l Cancer Institute | HIV-assoc. diseases |
| Lamivudine, 3TC | Glaxo Wellcome | HIV infection, |
| | | AIDS, ARC |
| | | (reverse |
| | | transcriptase |
| | | inhibitor); also |
| | | with AZT |
| Lobucavir | Bristol-Myers Squibb | CMV infection |
| Nelfinavir | Agouron | HIV infection, |
| | Pharmaceuticals | AIDS, ARC |
| | | (protease inhibitor) |
| Nevirapine | Boeheringer | HIV infection, |
| | Ingleheim | AIDS, ARC |
| | | (RT inhibitor) |
| Novapren | Novaferon Labs, Inc. | HIV inhibitor |
| | (Akron, OH) | |
| Peptide T | Peninsula Labs | AIDS |
| Octapeptide | (Belmont, CA) | |
| Sequence | | |
| Trisodium | Astra Pharm. | CMV retinitis, HIV |
| Phosphonoformate | Products, Inc. | infection, other CMV |
| | | infections |
| PNU-140690 | Pharmacia Upjohn | HIV infection, |
| | | AIDS, ARC |
| | | (protease inhibitor) |
| Probucol | Vyrex | HIV infection, AIDS |
| RBC-CD4 | Sheffield Med. | HIV infection, |
| | Tech (Houston, TX) | AIDS, ARC |
| Ritonavir | Abbott | HIV infection, |
| | | AIDS, ARC |
| | | (protease inhibitor) |
| Saquinavir | Hoffmann- | HIV infection, |
| | LaRoche | AIDS, ARC |
| | | (protease inhibitor) |
| Stavudine; d4T | Bristol-Myers Squibb | HIV infection, AIDS, |
| Didehydrodeoxy- | | ARC |
| Thymidine | | |
| Tipranavir | Boehringer Ingelheim | HIV infection, AIDS, ARC |
| | | (protease inhibitor) |
| Valaciclovir | Glaxo Wellcome | Genital HSV & CMV |
| | | infections |
| Virazole | Viratek/ICN | asymptomatic HIV |
| Ribavirin | (Costa Mesa, CA) | positive, LAS, ARC |
| VX-478 | Vertex | HIV infection, AIDS, |
| | | ARC |
| Zalcitabine | Hoffmann-LaRoche | HIV infection, AIDS, |
| | | ARC, with AZT |
| Zidovudine; AZT | Glaxo Wellcome | HIV infection, AIDS, |
| | | ARC, Kaposi's |
| | | sarcoma, in combination with |
| | | other therapies |
| Tenofovir disoproxil, | Gilead | HIV infection, |
| fumarate salt (VIREAD ®) | | AIDS, |
| | | (reverse transcriptase |
| | | inhibitor) |
| EMTRIVA ® (Emtricitabine) | Gilead | HIV infection, |
| (FTC) | | AIDS, |
| | | (reverse transcriptase |
| | | inhibitor) |
| COMBIVIR ® | GSK | HIV infection, |
| | | AIDS, |
| | | (reverse transcriptase |
| | | inhibitor) |
| Abacavir succinate | GSK | HIV infection, |
| (or ZIAGEN ®) | | AIDS, |
| | | (reverse transcriptase |
| | | inhibitor) |
| REYATAZ ® | Bristol-Myers Squibb | HIV infection |
| (or atazanavir) | | AIDs, protease |
| | | inhibitor |
| FUZEON ® | Roche/Trimeris | HIV infection |
| (Enfuvirtide or T-20) | | AIDs, viral Fusion |
| | | inhibitor |
| LEXIVA ® | GSK/Vertex | HIV infection |
| (or Fosamprenavir calcium) | | AIDs, viral protease |
| | | inhibitor |
| Selzentry | | |
| Maraviroc; (UK 427857) | Pfizer | HIV infection |
| | | AIDs, (CCR5 antagonist, in |
| | | development) |
| TRIZIVIR ® | GSK | HIV infection |
| | | AIDs, (three drug combination) |
| Sch-417690 (vicriviroc) | Schering-Plough | HIV infection |
| | | AIDs, (CCR5 antagonist, in |
| | | development) |
| TAK-652 | Takeda | HIV infection |
| | | AIDs, (CCR5 antagonist, in |
| | | development) |
| GSK 873140 | GSK/ONO | HIV infection |
| (ONO-4128) | | AIDs, (CCR5 antagonist, |
| | | in development) |
| Integrase Inhibitor | Merck | HIV infection |
| MK-0518 | | AIDs |
| Raltegravir | | |
| TRUVADA ® | Gilead | Combination of Tenofovir |
| | | disoproxil fumarate salt |
| | | (VIREAD ®) and EMTRIVA ® |
| | | (Emtricitabine) |
| Integrase Inhibitor | Gilead/Japan Tobacco | HIV Infection |
| GS917/JTK-303 | | AIDs |
| Elvitegravir | | in development |
| Triple drug combination | Gilead/Bristol-Myers Squibb | Combination of Tenofovir |
| ATRIPLA ® | | disoproxil fumarate salt |
| | | (VIREAD ®), EMTRIVA ® |
| | | (Emtricitabine), and |
| | | SUSTIVA ® (Efavirenz) |
| FESTINAVIR ® | Oncolys BioPharma | HIV infection |
| 4′-ethynyl-d4T | BMS | AIDs |
| | | in development |
| CMX-157 | Chimerix | HIV infection |
| Lipid conjugate of | | AIDs |
| nucleotide tenofovir | | |
| GSK1349572 | GSK | HIV infection |
| Integrase inhibitor | | AIDs |

IMMUNOMODULATORS

| | | |
|---|---|---|
| AS-101 | Wyeth-Ayerst | AIDS |
| Bropirimine | Pharmacia Upjohn | Advanced AIDS |
| Acemannan | Carrington Labs, Inc. | AIDS, ARC |
| | (Irving, TX) | |
| CL246,738 | Wyeth | AIDS, Kaposi's |
| | Lederle Labs | sarcoma |
| FP-21399 | Fuki ImmunoPharm | Blocks HIV fusion |
| | | with CD4+ cells |
| Gamma Interferon | Genentech | ARC, in combination |
| | | w/TNF (tumor |
| | | necrosis factor) |
| Granulocyte | Genetics Institute | AIDS |
| Macrophage Colony | Sandoz | |
| Stimulating Factor | | |
| Granulocyte | Hoechst-Roussel | AIDS |
| Macrophage Colony | Immunex | |
| Stimulating Factor | | |
| Granulocyte | Schering-Plough | AIDS, |
| Macrophage Colony | | combination |
| Stimulating Factor | | w/AZT |
| HIV Core Particle | Rorer | Seropositive HIV |
| Immunostimulant | | |
| IL-2 | Cetus | AIDS, in combination |
| Interleukin-2 | | w/AZT |
| IL-2 | Hoffman-LaRoche | AIDS, ARC, HIV, in |
| Interleukin-2 | Immunex | combination w/AZT |
| IL-2 | Chiron | AIDS, increase in |
| Interleukin-2 | | CD4 cell counts |
| (aldeslukin) | | |
| Immune Globulin | Cutter Biological | Pediatric AIDS, in |
| Intravenous | (Berkeley, CA) | combination w/AZT |
| (human) | | |
| IMREG-1 | Imreg | AIDS, Kaposi's |
| | (New Orleans, LA) | sarcoma, ARC, PGL |
| IMREG-2 | Imreg | AIDS, Kaposi's |
| | (New Orleans, LA) | sarcoma, ARC, PGL |
| Imuthiol Diethyl | Merieux Institute | AIDS, ARC |
| Dithio Carbamate | | |
| Alpha-2 | Schering Plough | Kaposi's sarcoma |
| Interferon | | w/AZT, AIDS |
| Methionine- | TNI Pharmaceutical | AIDS, ARC |
| Enkephalin | (Chicago, IL) | |
| MTP-PE | Ciba-Geigy Corp. | Kaposi's sarcoma |
| Muramyl-Tripeptide | | |
| Granulocyte | Amgen | AIDS, in combination |
| Colony Stimulating | | w/AZT |
| Factor | | |
| Remune | Immune Response | Immunotherapeutic |
| | Corp. | |
| rCD4 | Genentech | AIDS, ARC |
| Recombinant | | |
| Soluble Human CD4 | | |
| rCD4-IgG | | AIDS, ARC |
| hybrids | | |
| Recombinant | Biogen | AIDS, ARC |
| Soluble Human CD4 | | |
| Interferon | Hoffman-La Roche | Kaposi's sarcoma |
| Alfa 2a | | AIDS, ARC, |
| | | in combination w/AZT |
| SK&F106528 | Smith Kline | HIV infection |
| Soluble T4 | | |
| Thymopentin | Immunobiology | HIV infection |
| | Research Institute | |
| | (Annandale, NJ) | |
| Tumor Necrosis | Genentech | ARC, in combination |
| Factor; TNF | | w/gamma Interferon |

ANTI-INFECTIVES

| | | |
|---|---|---|
| Clindamycin with | Pharmacia Upjohn | PCP |
| Primaquine | | |
| Fluconazole | Pfizer | Cryptococcal |
| | | meningitis, |
| | | candidiasis |
| Pastille | Squibb Corp. | Prevention of |
| Nystatin Pastille | | oral candidiasis |
| Ornidyl | Merrell Dow | PCP |
| Eflornithine | | |
| Pentamidine | LyphoMed | PCP treatment |
| Isethionate (IM & IV) | (Rosemont, IL) | |
| Trimethoprim | | Antibacterial |
| Trimethoprim/sulfa | | Antibacterial |
| Piritrexim | Burroughs Wellcome | PCP treatment |
| Pentamidine | Fisons Corporation | PCP prophylaxis |
| Isethionate for | | |
| Inhalation | | |
| Spiramycin | Rhone-Poulenc | Cryptosporidial |
| | diarrhea | |
| Intraconazole- | Janssen-Pharm. | Histoplasmosis; |
| R51211 | | cryptococcal |
| | | meningitis |
| Trimetrexate | Warner-Lambert | PCP |
| Daunorubicin | NeXstar, Sequus | Kaposi's sarcoma |
| Recombinant Human | Ortho Pharm. Corp. | Severe anemia |
| Erythropoietin | | assoc. with AZT |
| | | therapy |
| Recombinant Human | Serono | AIDS-related |
| Growth Hormone | | wasting, cachexia |
| Megestrol Acetate | Bristol-Myers Squibb | Treatment of |
| | | anorexia assoc. |
| | | W/AIDS |
| Testosterone | Alza, Smith Kline | AIDS-related wasting |
| Total Enteral | Norwich Eaton | Diarrhea and |
| Nutrition | Pharmaceuticals | malabsorption |
| | | related to AIDS |

Additionally, the compounds of the disclosure herein set forth may be used in combination with HIV entry inhibitors. Examples of such HIV entry inhibitors are discussed in DRUGS OF THE FUTURE 1999, 24(12), pp. 1355-1362; CELL, Vol. 9, pp. 243-246, Oct. 29, 1999; and DRUG DISCOVERY TODAY, Vol. 5, No. 5, May 2000, pp. 183-194 and Inhibitors of the entry of HIV into host cells, Meanwell, Nicholas A.; Kadow, John F. Current Opinion in Drug Discovery & Development (2003), 6(4), 451-461. Specifically the compounds can be utilized in combination with attachment inhibitors, fusion inhibitors, and chemokine receptor antagonists aimed at either the CCR5 or CXCR4 coreceptor. HIV attachment inhibitors are also set forth in U.S. Pat. No. 7,354,924 and US 2005/0209246.

It will be understood that the scope of combinations of the compounds of this application with AIDS antivirals, immunomodulators, anti-infectives, HIV entry inhibitors or vaccines is not limited to the list in the above Table but includes, in principle, any combination with any pharmaceutical composition useful for the treatment of AIDS.

Preferred combinations are simultaneous or alternating treatments with a compound of the present disclosure and an inhibitor of HIV protease and/or a non-nucleoside inhibitor of HIV reverse transcriptase. An optional fourth component in the combination is a nucleoside inhibitor of HIV reverse transcriptase, such as AZT, 3TC, ddC or ddI. A preferred inhibitor of HIV protease is REYATAZ® (active ingredient Atazanavir). Typically a dose of 300 to 600 mg is administered once a day. This may be co-administered with a low dose of Ritonavir (50 to 500 mgs). Another preferred inhibitor of HIV protease is KALETRA®. Another useful inhibitor of HIV protease is indinavir, which is the sulfate salt of N-(2(R)-hydroxy-1-(S)-indanyl)-2(R)-phenylmethyl-4-(S)-hydroxy-5-(1-(4-(3-pyridyl-methyl)-2(S)—N′-(t-butylcarboxamido)-piperazinyl))-pentaneamide ethanolate, and is synthesized according to U.S. Pat. No. 5,413,999. Indinavir is generally administered at a dosage of 800 mg three times a day. Other preferred protease inhibitors are nelfinavir and ritonavir. Another preferred inhibitor of HIV protease is saquinavir which is administered in a dosage of 600 or 1200 mg tid. Preferred non-nucleoside inhibitors of HIV reverse transcriptase include efavirenz. These combinations may have unexpected effects on limiting the spread and degree of infection of HIV. Preferred combinations include those with the following (1) indinavir with efavirenz, and, optionally, AZT and/or 3TC and/or ddI and/or ddC; (2) indinavir, and any of AZT and/or ddI and/or ddC and/or 3TC, in particular, indinavir and AZT and 3TC; (3) stavudine and 3TC and/or zidovudine; (4) tenofovir disoproxil fumarate salt and emtricitabine.

In such combinations the compound of the present invention and other active agents may be administered separately or in conjunction. In addition, the administration of one element may be prior to, concurrent to, or subsequent to the administration of other agent(s).

### Abbreviations:

### NBS=N-bromosuccinimine

TBDMS=tert-butyldimethylsilane
PTFE=polytetrafluoroethylene
NMO=4-methylmorpholine-N-oxide
THF=tetrahydrofuran
TLC=thin layer chromatography
DCM=dichloromethane
DCE=dichloroethane
TFA=trifluoroacetic acid
LCMS=liquid chromatography mass spectroscopy
Prep=preparative
HPLC=high performance liquid chromatography
DAST=(diethylamino)sulfur trifluoride
TEA=triethylamine

### DIPEA=N,N-diisopropylethylamine

HATU=[O-(7-azabenzotriazol-1-yl)-1,1,3,3-tetramethyluronium hexafluorophosphate]
DMAP=dimethylaminopyridine
TMS=trimethylsilyl
NMR=nuclear magnetic resonance
DPPA=diphenyl phosphoryl azide
AIBN=azobisisobutyronitrile
TBAF=tetrabutylammonium fluoride
DMF=dimethylformamide
TBTU=O-(benzotriazol-1-yl)-N,N,N′,N′-tetramethyluronium tetrafluoroborate
min=minute(s)
h=hour(s)
sat.=saturated
TEA=triethylamine
EtOAc=ethyl acetate
TFA=trifluoroacetic acid
PCC=pyridinium chlorochromate
TLC=thin layer chromatography
Tf₂NPh=(trifluoromethylsulfonyl)methanesulfonamide
dioxane=1,4-dioxane
PG=protective group
atm=atmosphere(s)
mol=mole(s)
mmol=milimole(s)
mg=milligram(s)
μg=microgram(s)
μl=microliter(s)
μm=micrometer(s)
mm=millimeter(s)

### EXAMPLES

The following examples illustrate typical syntheses of the compounds of Formulas I and II, as described generally above. These examples are illustrative only and are not intended to limit the disclosure in any way. The reagents and starting materials are readily available to one of ordinary skill in the art.

### Chemistry

### Typical Procedures and Characterization of Selected Examples:

Unless otherwise stated, solvents and reagents were used directly as obtained from commercial sources, and reactions were performed under a nitrogen atmosphere. Flash chromatography was conducted on Silica gel 60 (0.040-0.063 particle size; EM Science supply). ¹H NMR spectra were recorded on Bruker DRX-500f at 500 MHz (or Bruker AV 400 MHz, Bruker DPX-300B or Varian Gemini 300 at 300 MHz as stated). The chemical shifts were reported in ppm on the δ scale relative to δTMS=0. The following internal references were used for the residual protons in the following solvents: CDCl₃ δ_{H} 7.26), CD₃OD (δ_{H} 3.30), Acetic-d4 (Acetic Acid d₄) (δ_{H} 11.6, 2.07), DMSO mix or DMSO-D6_CDCl₃ ((_{H} 2.50 and 8.25) (ratio 75%:25%), and DMSO-D6 (δ_{H} 2.50). Standard acronyms were employed to describe the multiplicity patterns: s (singlet), br. s (broad singlet), d (doublet), t (triplet), q (quartet), m (multiplet), b (broad), app (apparent). The coupling constant (J) is in Hertz. All Liquid Chromatography (LC) data were recorded on a Shimadzu LC-10AS liquid chromatograph using a SPD-10AV UV-Vis detector with Mass Spectrometry (MS) data determined using a Micromass Platform for LC in electrospray mode.

LC/MS methods:

### Method 1

Start % B=0, Final % B=100 over 2 minute gradient, hold at 100% B
Flow Rate=4 mL/min

### Wavelength=220 nm

Solvent A=95% water, 5% methanol, 10 mM ammonium acetate
Solvent B=5% water, 95% methanol, 10 mM ammonium acetate

### Column=Xbridge C18 5 μm 4.6×50 mm

### Method 2

Start % B=0, Final % B=100 over 2 minute gradient, hold at 100% B
Flow Rate=4 mL/min

### Wavelength=220 nm

Solvent A=95% water, 5% methanol, 10 mM ammonium acetate
Solvent B=5% water, 95% methanol, 10 mM ammonium acetate

### Column=Phenomenex Luna C18, 5 μm, 3.0×50 mm

### Method 3

Start % B=0, Final % B=100 over 2 minute gradient, hold at 100% B
Flow Rate=1 mL/min

### Wavelength=220 nm

Solvent A=95% water, 5% acetonitrile, 10 mM ammonium acetate
Solvent B=5% water, 95% acetonitrile, 10 mM ammonium acetate

### Column=Phenomenex Luna C18, 3 μm, 2.0×30 mm

### Method 4

Start % B=0, Final % B=100 over 2 minute gradient, hold at 100% B
Flow Rate=1 mL/min

### Wavelength=220 nm

Solvent A=95% water, 5% methanol, 10 mM ammonium acetate
Solvent B=5% water, 95% methanol, 10 mM ammonium acetate

### Column=Phenomenex Luna C18, 3 μm, 2.0×30 mm

### Method 5

Start % B=0, Final % B=100 over 2 minute gradient, hold at 100% B
Flow Rate=1 mL/min

### Wavelength=220 nm

Solvent A=90% water, 10% methanol, 0.1% TFA
Solvent B=10% water, 90% methanol, 0.1% TFA

### Column=Phenomenex Luna C18, 3 μm, 2.0×30 mm

### Method 6

Start % B=0, Final % B=100 over 2 minute gradient, hold at 100% B
Flow Rate=1 mL/min

### Wavelength=220 nm

Solvent A=90% water, 10% Acetonitrile, 0.1% TFA
Solvent B=10% Acetonitrile, 90% water, 0.1% TFA

### Column=Phenomenex Luna C18, 3 μm, 2.0×30 mm

### Prep HPLC Methods:

### Prep HPLC Method 1

Start % B=25 Final % B=100 over 10 minute gradient, hold at 100% B
Flow Rate=25 mL/min

### Solvent A=5% MeOH-95% H₂O-10 mM Ammonium Acetate

### Solvent B=95% MeOH-5% H₂O-10 mM Ammonium Acetate

### Column=XBridge Phenyl 19×100 mm

### Prep HPLC Method 2

Start % B=0, Final % B=100 over 20 minute gradient, hold at 100% B for 4 minutes

Flow Rate=50 mL/min
Solvent A=90% water, 10% acetonitrile, 0.1% TFA
Solvent B=10% water, 90% acetonitrile, 0.1% TFA

### Column=Waters Sunfire C18, 5 μm, 30×150 mm

### Prep HPLC Method 3

Start % B=0, Final % B=100 over 15 minute gradient, hold at 100% B for 4 minutes
Flow Rate=50 mL/min
Solvent A=90% water, 10% acetonitrile, 0.1% TFA
Solvent B=10% water, 90% acetonitrile, 0.1% TFA

### Column=Waters Sunfire C18, 5 μm, 30×150 mm

### Prep HPLC Method 4

Start % B=15, Final % B=100 over 20 minute gradient, hold at 100% B for 4 minutes
Flow Rate=50 mL/min
Solvent A=90% water, 10% acetonitrile, 0.1% TFA
Solvent B=10% water, 90% acetonitrile, 0.1% TFA

### Column=Waters Sunfire C18, 5 μm, 30×150 mm

### Prep HPLC Method 5

Start % B=15, Final % B=100 over 10 minute gradient, hold at 100% B for 4 minutes
Flow Rate=50 mL/min
Solvent A=90% water, 10% acetonitrile, 0.1% TFA
Solvent B=10% water, 90% acetonitrile, 0.1% TFA

### Column=Waters Sunfire C18, 5 μm, 30×150 mm

### Prep HPLC Method 6

Start % B=15, Final % B=100 over 30 minute gradient, hold at 100% B for 4 minutes
Flow Rate=50 mL/min
Solvent A=90% water, 10% acetonitrile, 0.1% TFA
Solvent B=10% water, 90% acetonitrile, 0.1% TFA

### Column=Waters Sunfire C18, 5 μm, 30×150 mm

### Prep HPLC Method 7

Start % B=15, Final % B=100 over 20 minute gradient, hold at 100% B for 4 minutes
Flow Rate=50 mL/min
Solvent A=95% water, 5% acetonitrile, 10 mM ammonium acetate
Solvent B=5% water, 95% acetonitrile, 10 mM ammonium acetate

### Column=Waters Sunfire C18, 5 μm, 30×150 mm

### Prep HPLC Method 8

Start % B=15, Final % B=100 over 30 minute gradient, hold at 100% B for 15 minutes

Flow Rate=50 mL/min
Solvent A=90% water, 10% acetonitrile, 0.1% TFA
Solvent B=10% water, 90% acetonitrile, 0.1% TFA

### Column=Waters Sunfire C18, 5 μm, 30×150 mm

### Prep HPLC Method 9

Start % B=25 Final % B=100 over 10 minute gradient, hold at 100% B

### Solvent A=5% MeOH-95% H₂O-10 mM Ammonium Acetate

Flow Rate=25 mL/min

### Solvent B=95% MeOH-5% H₂O-10 mM Ammonium Acetate

### Column=Waters-Sunfire OBD 19×100 mm S5

### Prep HPLC Method 10

Start % B=20, Final % B=100 over 20 minute gradient, hold at 100% B for 5 minutes
Flow Rate=50 mL/min
Solvent A=90% water, 10% acetonitrile, 0.1% TFA
Solvent B=10% water, 90% acetonitrile, 0.1% TFA

### Column=Waters Sunfire C18, 5 μm, 30×150 mm

### Prep HPLC Method 11

Start % B=20, Final % B=100 over 30 minute gradient, hold at 100% B for 4 minutes
Flow Rate=50 mL/min
Solvent A=90% water, 10% acetonitrile, 0.1% TFA
Solvent B=10% water, 90% acetonitrile, 0.1% TFA

### Column=Waters Sunfire C18, 5 μm, 30×150 mm

### Prep HPLC Method 12

Start % B=0, Final % B=100 over 20 minute gradient, hold at 100% B for 4 minutes
Flow Rate=50 mL/min
Solvent A=95% water, 5% acetonitrile, 10 mM ammonium acetate
Solvent B=5% water, 95% acetonitrile, 10 mM ammonium acetate

### Column=Waters Sunfire C18, 5 μm, 30×150 mm

### Prep HPLC Method 13

Start % B=0, Final % B=100 over 15 minute gradient, hold at 100% B for 4 minutes
Flow Rate=50 mL/min
Solvent A=95% water, 5% acetonitrile, 10 mM ammonium acetate
Solvent B=5% water, 95% acetonitrile, 10 mM ammonium acetate

### Column=Waters Sunfire C18, 5 μm, 30×150 mm

### Prep HPLC Method 14

Start % B=0, Final % B=100 over 10 minute gradient, hold at 100% B for 4 minutes
Flow Rate=50 mL/min
Solvent A=90% water, 10% acetonitrile, 0.1% TFA
Solvent B=10% water, 90% acetonitrile, 0.1% TFA

### Column=Waters Sunfire C18, 5 μm, 30×150 mm

### Prep HPLC Method 15

Start % B=0, Final % B=100 over 20 minute gradient, hold at 100% B for 4 minutes
Flow Rate=50 mL/min
Solvent A=90% water, 10% acetonitrile, 0.1% TFA
Solvent B=10% water, 90% acetonitrile, 0.1% TFA

### Column=Waters Xbridge Phenyl, 5 μm, 30×100 mm

### Prep HPLC Method 16

Start % B=0, Final % B=100 over 30 minute gradient, hold at 100% B for 4 minutes
Flow Rate=50 mL/min
Solvent A=90% water, 10% acetonitrile, 0.1% TFA
Solvent B=10% water, 90% acetonitrile, 0.1% TFA

### Column=Waters Sunfire C18, 5 μm, 30×150 mm

### Prep HPLC Method 17

Start % B=0, Final % B=100 over 30 minute gradient, hold at 100% B for 4 minutes
Flow Rate=50 mL/min
Solvent A=95% water, 5% MeOH, 10 mM ammonium bicarbonate
Solvent B=5% water, 95% MeOH, 10 mM ammonium bicarbonate

### Column=Waters Sunfire C18, 5 μm, 30×150 mm

### Prep HPLC Method 18

Start % B=20 Final % B=60 over 10 minute gradient, hold at 100% B for 5 min
Flow Rate=20 mL/min

### Solvent A=Water—20 mM Ammonium Acetate

### Solvent B=95% Acetonitrile—5% H₂O-20 mM Ammonium Acetate

### Column=XBridge Phenyl C18 19×200 mm S5

### Prep HPLC Method 19

Start % B=0, Final % B=100 over 40 minute gradient, hold at 100% B for 4 minutes

Flow Rate=50 mL/min
Solvent A=90% water, 10% acetonitrile, 0.1% TFA
Solvent B=10% water, 90% acetonitrile, 0.1% TFA

### Column=Waters Sunfire C18, 5 μm, 30×150 mm

### Prep HPLC Method 20

Start % B=30 Final % B=100 over 15 minute gradient, hold at 100% B
Flow Rate=25 mL/min

### Solvent A=5% MeOH-95% H₂O-10 mM Ammonium Acetate

### Solvent B=95% MeOH-5% H₂O-10 mM Ammonium Acetate

### Column=XBridge Phenyl 19×100 mm S5

### Prep HPLC Method 21

Start % B=15 Final % B=90 over 20 minute gradient, hold at 100% B
Flow Rate=40 mL/min
Solvent A=90% water—10% acetonitrile, 0.1% TFA
Solvent B=10% water—90% acetonitrile, 0.1% TFA

### Column=Waters Sunfire C18, 5 μM, 30×100 mm

### Prep HPLC Method 22

Start % B=25 Final % B=90 over 15 minute gradient, hold at 100% B
Flow Rate=40 mL/min
Solvent A=90% water—10% acetonitrile, 0.1% TFA
Solvent B=10% water—90% acetonitrile, 0.1% TFA

### Column=Waters Sunfire C18, 5 μM, 30×100 mm

### Prep HPLC Method 23

Start % B=30, Final % B=100 over 20 minute gradient, hold at 100% B for 10 minutes
Flow Rate=50 mL/min
Solvent A=90% water, 10% acetonitrile, 0.1% TFA
Solvent B=10% water, 90% acetonitrile, 0.1% TFA

### Column=Waters Sunfire C18, 5 μm, 30×100 mm

### Prep HPLC Method 24

Start % B=30, Final % B=100 over 12 minute gradient, hold at 100% B for 8 minutes
Flow Rate=50 mL/min
Solvent A=90% water, 10% acetonitrile, 0.1% TFA
Solvent B=10% water, 90% acetonitrile, 0.1% TFA

### Column=Waters Sunfire C18, 5 μm, 30×100 mm

### Prep HPLC Method 25

Start % B=20, Final % B=100 over 15 minute gradient, hold at 100% B for 5 minutes
Flow Rate=50 mL/min
Solvent A=90% water, 10% acetonitrile, 0.1% TFA
Solvent B=10% water, 90% acetonitrile, 0.1% TFA

### Column=Waters Xbridge Phenyl, 5 μm, 30×100 mm

### Prep HPLC Method 26

Start % B=20, Final % B=100 over 10 minute gradient, hold at 100% B for 15 minutes
Flow Rate=50 mL/min
Solvent A=90% water, 10% acetonitrile, 0.1% TFA
Solvent B=10% water, 90% acetonitrile, 0.1% TFA

### Column=Waters Xbridge Phenyl, 5 μm, 30×100 mm

### Preparation of Key Intermediates:

Intermediates 1-4 can be prepared as shown in the following scheme:

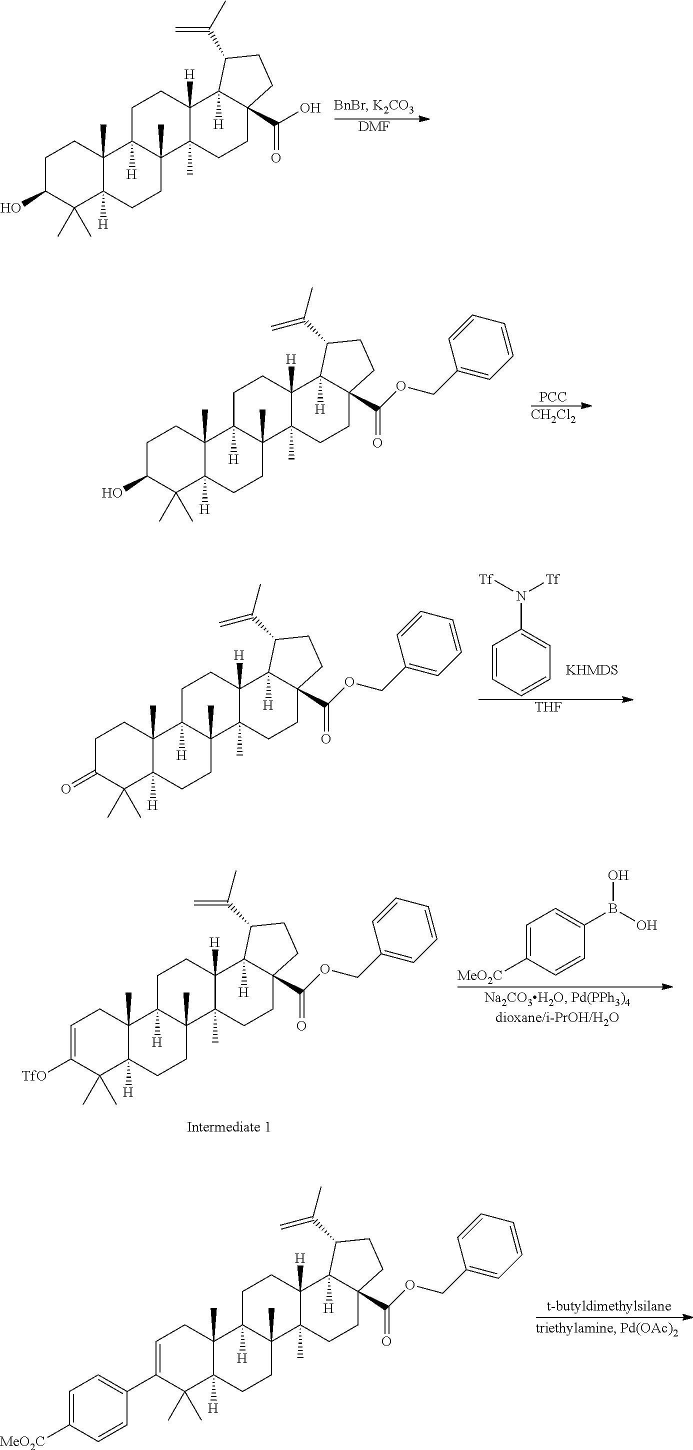

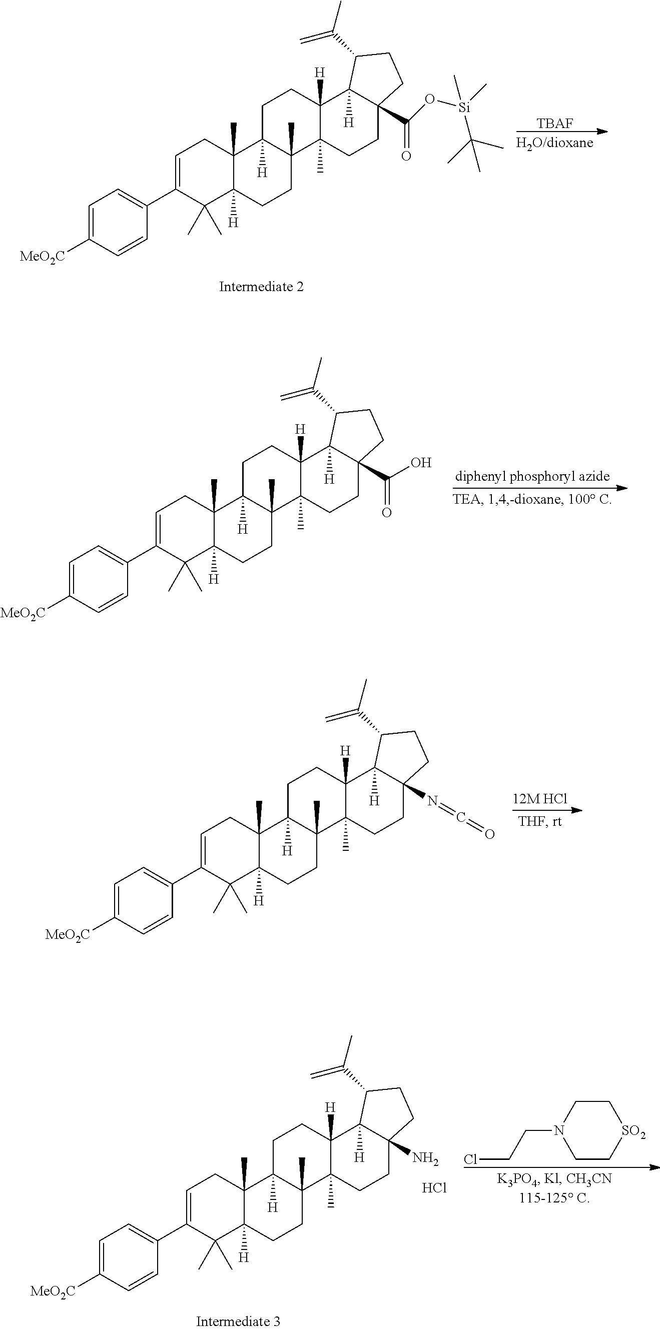

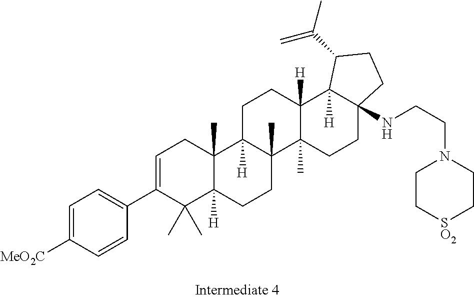

### Preparation of (1R,3aS,5aR,5bR,7aR,9S,11aR,11bR,13aR,13bR)-benzyl 9-hydroxy-5a,5b,8,8,11a-pentamethyl-1-(prop-1-en-2-yl)icosahydro-1H-cyclopenta[a]chrys ene-3a-carboxylate

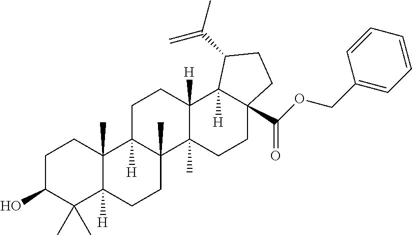

To a suspension of betulinic acid (12 g, 26.3 mmol) and potassium carbonate (7.26 g, 52.6 mmol) in DMF (150 mL) was added benzyl bromide (3.28 mL, 27.6 mmol). The mixture was heated to 60° C. for 3.5 h, and then it was cooled to rt. Solids started to precipitate upon cooling. The mixture was diluted water (200 mL) and the solids that formed were collected by filtration to give the title compound (13.92 g, 25.5 mmol, 97% yield) as a white solid. ¹H NMR (500 MHz, CHLOROFORM-d) δ ppm 7.39-7.28 (m, 5H), 5.16-5.06 (m, 2H), 4.71 (d, J=1.83 Hz, 1H), 4.59 (s, 1H), 3.17 (ddd, J=11.44, 5.65, 5.49 Hz, 1H), 3.01 (td, J=10.99, 4.88 Hz, 1H), 2.27 (ddd, J=12.36, 3.20, 3.05Hz, 1H), 2.21-2.13 (m, 1H), 1.93-1.81 (m, 2H), 1.67 (s, 3H), 0.95 (s, 3H), 0.93 (s, 3H), 1.71-0.82 (m, 20H), 0.79 (s, 3H), 0.75 (s, 3H), 0.74 (s, 3H).

### Preparation of (1R,3aS,5aR,5bR,7aR,11aR,11bR,13aR,13bR)-benzyl 5a,5b,8,8,11a-pentamethyl-9-oxo-1-(prop-1-en-2-yl)icosahydro-1H-cyclopenta[α]chrysene-3a-carboxylate

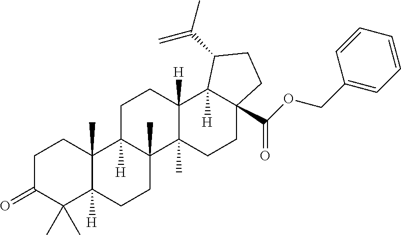

To a solution of (1R,3aS,5aR,5bR,7aR,9S,11aR,11bR,13aR,13bR)-benzyl 9-hydroxy-5a,5b,8,8,11a-pentamethyl-1-(prop-1-en-2-yl)icosahydro-1H-cyclopenta[a]chrysene-3a-carboxylate (7.1 g, 12.98 mmol) in dichloromethane (100 mL) was added PCC (4.20 g, 19.48 mmol). After stirring for five minutes, the mixture turned a deep crimson color. The mixture was further stirred for 5.5 h. The mixture was filtered through a pad of celite and silica gel which was washed with dichloromethane and then with a 1:1 mixture of ethyl acetate:hexanes. The filtrate was concentrated under reduced pressure to give the title compound (6.92 g, 12.7 mmol, 98% yield) as a white foam. ¹H NMR (500 MHz, CHLOROFORM-d) δ ppm 7.38-7.28 (m, 5H), 5.17-5.06 (m, 2H), 4.72 (d, J=1.83 Hz, 1H), 4.59 (s, 1H), 3.01 (td, J=10.99, 4.88 Hz, 1H), 2.51-2.43 (m, 1

H), 2.42-2.34 (m, 1H), 2.28 (dt, J=12.59, 3.17 Hz, 1H), 2.21 (td, J=12.28, 3.51 Hz, 1H), 1.94-1.82 (m, 3H), 1.67 (s, 3H), 1.05 (s, 3H), 1.01 (s, 3H), 1.73-0.95 (m, 17H), 0.94 (s, 3H), 0.89 (s, 3H), 0.78 (s, 3H).

### Preparation of (1R,3aS,5aR,5bR,7aR,11aR,11bR,13aR,13bR)-benzyl 5a,5b,8,8,11a-pentamethyl-1-(prop-1-en-2-yl)-9-(trifluoromethylsulfonyloxy)-2,3,3a,4,5,5a,5b,6,7,7a,8,11,11a,11b,12,13,13a,13b-octadecahydro-1H-cyclopenta[a]chrysene-3a-carboxylate. Intermediate 1

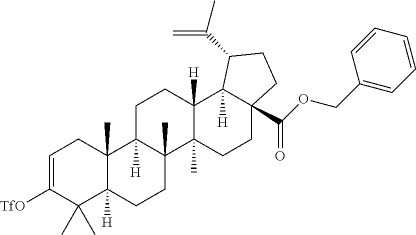

A solution of (1R,3aS,5aR,5bR,7aR,11aR,11bR,13aR,13bR)-benzyl 5a,5b,8,8,11a-pentamethyl-9-oxo-1-(prop-1-en-2-yl)icosahydro-1H-cyclopenta[a]chrysene-3a-carboxylate (29.0 g, 53.2 mmol) in THF (200 mL) was cooled to −78° C. To the solution was added KHMDS (0.5 M in toluene) (213 mL, 106 mmol). The yellow solution was stirred at −78° C. for 25 minutes and a solution of 1,1,1-trifluoro-N-phenyl-N-(trifluoromethyl)sulfonyl methanesulfonamide (20.92 g, 58.6 mmol) in THF (70 mL) and toluene (30 mL) was added via cannula. The solution was stirred at −78° C. for 3 h. Then, an additional 1.0 g of 1,1,1-trifluoro-N-phenyl-N-(trifluoromethyl)sulfonyl methanesulfonamide was added and the mixture was stirred at −78° C. After stirring for 1 h, the mixture was quenched with water (300 mL) and the mixture was extracted with ethyl acetate (3×200 mL). The combined organic layers were dried with MgSO₄. The drying agent was removed by filtration, and the filtrate was concentrated under reduced pressure to give the title compound (40.0 g, 59.1 mmol) as a yellow solid. Product Rf=0.57 by silica gel TLC, 5% EtOAc in hexanes, visualized using Hanessian's stain. ¹H NMR (500 MHz, CHLOROFORM-d) δ ppm 7.29-7.41 (m, 5H), 5.54 (dd, J=6.71, 1.53 Hz, 1H), 5.13-5.18 (m, 1H), 5.05-5.12 (m, 1H), 4.72 (d, J=1.53 Hz, 1H), 4.59 (s, 1H), 3.02 (td, J=10.99, 4.58 Hz, 1H), 2.25-2.31 (m, 1H), 2.22 (td, J=12.21, 3.36 Hz, 1H), 2.14 (dd, J=17.09, 6.71 Hz, 1H), 1.81-1.96 (m, 2H), 1.67 (s, 3H), 1.10 (s, 3H), 1.00 (s, 3H), 0.94 (s, 3H), 0.91-1.77 (m, 17H), 0.88 (s, 3H), 0.77 (s, 3H).

### Preparation of (1R,3aS,5aR,5bR,7aR,11aS,11bR,13aR,13bR)-benzyl 9-(4-(methoxycarbonyl)phenyl)-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysene-3a-carboxylate

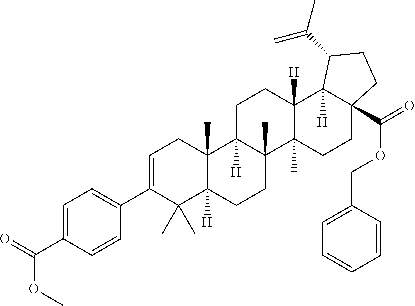

To a solution of (1R,3aS,5aR,5bR,7aR,11aR,11bR,13bR)-benzyl 5a,5b,8,8,11a-pentamethyl-1-(prop-1-en-2-yl)-9-(trifluoromethylsulfonyloxy)-2,3,3a,4,5,5a,5b,6,7,7a,8,11,11a,11b,12,13,13a,13b-octadecahydro-1H-cyclopenta[a]chrysene-3a-carboxylate (6.21 g, 9.18 mmol) in dioxane (25 mL) was added 2-propanol (25 mL) and water (15 mL) followed by sodium carbonate monohydrate (3.42 g, 27.5 mmol), 4-methoxycarbonylphenylboronic acid (2.478 g, 13.77 mmol), and tetrakis(triphenylphosphine)palladium(0) (0.318 g, 0.275 mmol). The flask was attached to a reflux condenser, flushed with N₂ and heated to reflux overnight. The mixture was then cooled to rt and diluted with water (75 mL). The mixture was extracted with ethyl acetate (3×75 mL) and washed with brine (75 mL). The combined organic layers were dried with MgSO₄, filtered, and concentrated under reduced pressure. The residue was adsorbed to silica gel and purified by silica gel flash chromatography using a 0-20% ethyl acetate in hexanes gradient. The fractions containing the expected product were combined and concentrated under reduced pressure to give the title compound (4.16 g, 6.28 mmol, 68.4% yield) as a white foam. ¹H NMR (500 MHz, CHLOROFORM-d) δ ppm 7.92 (d, J=8.24 Hz, 2H), 7.40-7.29 (m, 5H), 7.19 (d, J=8.24 Hz, 2H), 5.28 (dd, J=6.10, 1.83 Hz, 1H), 5.19-5.07 (m, 2H), 4.73 (d, J=1.83 Hz, 1H), 4.60 (s, 1H), 3.90 (s, 3H), 3.04 (td, J=10.91, 4.73 Hz, 1H), 2.20-2.32 (m, 2H), 2.09 (dd, J=17.24, 6.26 Hz, 1H), 1.95-1.82 (m, 2H), 1.69 (s, 3H), 0.97 (s, 3H), 0.95 (s, 3H), 0.92 (s, 3H), 0.91 (s, 3H), 1.75-0.87 (m, 17H), 0.82 (s, 3H).

### Preparation of (1R,3aS,5aR,5bR,7aR,11aS,11bR,13aR,13bR)-tert-butyldimethylsilyl 9-(4-(methoxycarbonyl)phenyl)-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysene-3a-carboxylate. Intermediate 2

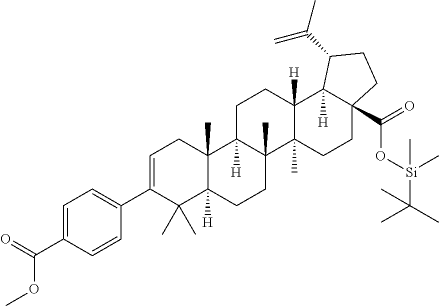

To a solution of (1R,3aS,5aR,5bR,7aR,11aS,11bR,13aR,13bR)-benzyl 9-(4-(methoxycarbonyl)phenyl)-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysene-3a-carboxylate (3.82 g, 5.76 mmol) in dichloroethane (100 mL) was added triethylamine (1.285 mL, 9.22 mmol), tert-butyldimethylsilane (1.912 mL, 11.52 mmol), and palladium(II) acetate (0.647 g, 2.88 mmol). The mixture was flushed with N₂ and heated to 60° C. After 2 h, the reaction was cooled to rt, filtered through a pad of celite and silica gel to remove the solids which were washed with 25% EtOAc in hexanes. The filtrate was concentrated under reduced pressure and treated with acetic acid (25 mL), THF (10 mL) and water (3 mL). After stirring for 1 h the solids formed were collected by filtration and washed with water to give the title compound (3.62 g, 5.27 mmol, 91% yield) as a white solid. ¹H NMR (400 MHz, CHLOROFORM-d) δ ppm 7.94 (d, J=8.28 Hz, 2H), 7.21 (d, J=8.28 Hz, 2H), 5.30 (dd, J=6.15, 1.63 Hz, 1H), 4.75 (d, J=1.76 Hz, 1H), 4.62 (s, 1H), 3.92 (s, 4H), 3.08 (td, J=10.92, 4.27 Hz, 1H), 2.35-2.22 (m, 2H), 2.17-2.06 (m, 1H), 2.02-1.84 (m, 2H), 1.71 (s, 3H), 1.01 (s, 6H), 0.99 (br. s., 3H), 0.98 (s, 9H), 0.94 (s, 6H), 1.78-0.90 (m, 16H), 0.32-0.28 (m, 6H).

### Preparation of (1R,3aS,5aR,5bR,7aR,11aS,11bR,13aR,13bR)-9-(4-(methoxycarbonyl)phenyl)-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysene-3a-carboxylic acid

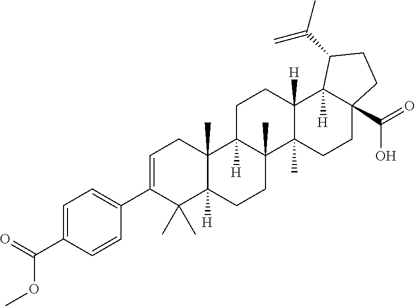

To solution of (1R,3aS,5aR,5bR,7aR,11aS,11bR,13aR,13bR)-tert-butyldimethylsilyl 9-(4-(methoxycarbonyl)phenyl)-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysene-3a-carboxylate (3.12 g, 4.54 mmol) in dioxane (25 mL) was added TBAF (75% wt in water) (2.375 g, 6.81 mmol) and the mixture was stirred at rt for 4 h. The reaction mixture was diluted with 1N HCl (25 mL) and water (5 mL) and extracted with dichloromethane (3×100 mL). The combined organic layers were dried over Na₂SO₄, filtered, and partially concentrated under reduced pressure to about 10 mL volume. To the partially concentrated mixture was added 1N HCl (50 mL). The solids that formed were collected by filtration and washed with water to give the title compound (2.58 g, 4.50 mmol, 99% yield) as a white solid. LCMS: m/e 571.47 (M−H)⁻, 3.60 min (method 2). ¹H NMR (500 MHz, CHLOROFORM-d) δ ppm 9.80 (br. s., 1H), 7.92 (d, J=8.24 Hz, 2H), 7.18 (d, J=8.24 Hz, 2H), 5.32-5.26 (m, 1H), 4.75 (s, 1H), 4.62 (br. s., 1H), 3.90 (s, 3H), 3.07-2.99 (m, 1H), 2.33-2.21 (m, 2H), 2.10 (dd, J=17.09, 6.10 Hz, 1H), 2.06-1.94 (m, 2H), 1.70 (s, 3H), 1.01 (br. s., 3H), 1.00 (br. s., 3H), 0.98 (s, 3H), 0.91 (s, 6H), 1.79-0.89 (m, 17H).

### Preparation of methyl 4-((1R,3aS,5aR,5bR,7aR,11aS,11bR,13aR,13bR)-3a-isocyanato-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysen-9-yl)benzoate

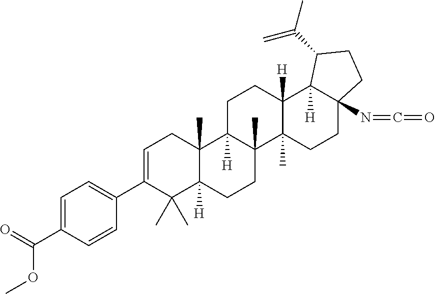

To a slurry of (1R,3aS,5aR,5bR,7aR,11aS,11bR,13aR,13bR)-9-(4-(methoxycarbonyl)phenyl)-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysene-3a-carboxylic acid (10 g, 17.46 mmol) in 1,4-dioxane (200 mL) was added triethylamine (4.38 mL, 31.4 mmol) followed by diphenyl phosphoryl azide (5.82 mL, 26.2 mmol). The resulting white slurry was heated to 100° C. After 5 h, the reaction was allowed to cool to rt and was then diluted with EtOAc and washed with 1N NaOH (2×70 mL). The combined aqueous layer was extracted with EtOAc (2×150 mL). The combined organic layer was dried over Na₂SO₄, filtered and concentrated to a slurry (75 mL) which was stored in a refrigerator overnight. The slurry was filtered and the white solid product was washed with Et₂O. The liquid filtrate was concentrated to a yellow slurry which was filtered and washed with Et₂O to give more white solid product. The two batches of white solid were combined and dried in vacuo to give the title compound (8.6 g, 15.09 mmol, 86% yield) as a white solid. ¹H NMR (500 MHz, CHLOROFORM-d) δ ppm 8.0 (2H, d, J=8.2Hz), 7.2 (2H, d, J=8.2Hz), 5.3 (1H, d, J=4.6 Hz), 4.8 (1H, s), 4.7 (1H, s), 3.9 (3H, s), 2.6 (1H, td, J=10.8, 5.8 Hz), 2.1-2.2 (2H, m), 1.8-2.0 (4H, m), 1.7-1.8 (1H, m), 1.7 (3H, s), 1.5-1.7 (5H, m), 1.4-1.5 (5H, m), 1.3-1.4 (2H, m), 1.2-1.3 (2H, m), 1.1 (3H, s), 1.1-1.1 (1H, m), 1.0 (3H, s), 1.0 (3H, s), 1.0 (3H, br. s.), 1.0 (3H, br. s.). ¹³C NMR (CHLOROFORM-d) δ ppm 14.2, 15.4, 16.2, 19.2, 19.5, 20.8, 21.0, 24.7, 27.4, 29.0, 29.2, 33.3, 36.0, 37.2, 39.0, 39.0, 40.3, 41.5, 41.8, 47.8, 49.0, 49.2, 51.7, 52.6, 66.8, 71.3, 110.2, 121.3, 123.7, 127.6, 128.2, 129.8, 146.0, 148.4, 148.6, 166.9.

### Preparation of methyl 4-((1R,3aS,5aR,5bR,7aR,11aS,11bR,13aR,13bR)-3a-amino-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysen-9-yl)benzoate. Intermediate 3

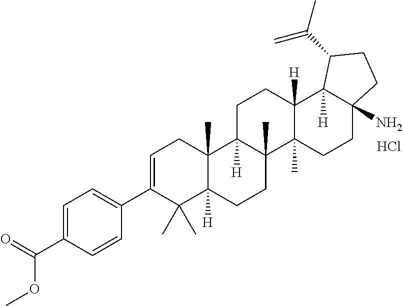

To a cloudy solution of methyl 4-((1R,3aS,5aR,5bR,7aR,11aS,11bR,13aR,13bR)-3a-isocyanato-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysen-9-yl)benzoate (5.47 g, 9.60 mmol) in THF (100 mL) was added concentrated hydrochloric acid (19.83 mL, 240 mmol). The resulting homogeneous mixture was stirred at rt for 72 h, the reaction mixture was concentrated to dryness to give methyl 4-((1R,3aS,5aR,5bR,7aR,11aS,11bR,13aR,13bR)-3a-amino-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysen-9-yl)benzoate, HCl (4.98 g, 8.58 mmol, 89% yield) as a white solid. LCMS: m/e 544.5 (M+H)⁺, 3.26 min (method 3). ¹H NMR (500 MHz, 1:1 CDCl₃:MeOD, MeOD lock) δ ppm 7.9 (2H, d, J=8.5Hz), 7.3 (1H, t, J=7.8 Hz), 7.2 (2H, d, J=8.5Hz), 7.1 (1H, t, J=7.3 Hz), 5.3 (1H, d, J=4.6 Hz), 4.8 (1H, s), 4.7 (1H, br. s.), 3.9 (2H, s), 3.6 (2H, dt, J=15.6, 6.6 Hz), 3.3 (1H, dt, J=3.1, 1.6 Hz), 2.6 (1H, td, J=11.0, 6.1 Hz), 2.1 (1H, dd, J=17.1, 6.4 Hz), 2.0 (1H, d, J=13.4 Hz), 1.9-2.0 (1H, m), 1.8-1.9 (2H, m), 1.7-1.7 (3H, m), 1.6-1.7 (3H, m), 1.5-1.6 (3H, m), 1.5-1.5 (2H, m), 1.4 (1H, br. s.), 1.3-1.4 (1H, m), 1.2-1.3 (1H, m), 1.1-1.2 (2H, m), 1.1-1.1 (1H, m), 1.0 (3H, s), 1.0 (3H, s), 0.9 (3H, s), 0.9 (3H, s).

### Preparation of methyl 4-((1R,3aS,5aR,5bR,7aR,11aS,11bR,13aR,13bR)-3a-((2-(1,1-dioxido-4-thiomorpholinyl)ethyl)amino)-1-isopropenyl-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate. Intermediate 4

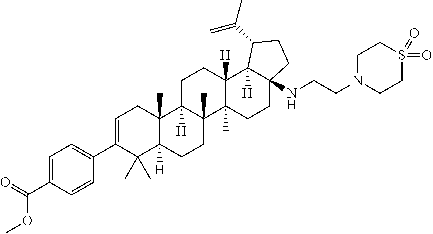

A mixture of methyl 4-((1R,3aS,5aR,5bR,7aR,11aS,11bR,13aR,13bR)-3a-amino-1-isopropenyl-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (600 mg, 1.10 mmol), 4-(2-chloroethyl)thiomorpholine 1,1-dioxide (600 mg, 2.56 mmol) (prepared as described in WO2002045652), anhydrous potassium phosphate (3.00 g, 14.1 mmol) and potassium iodide (10 mg, 0.060 mmol) in acetonitrile (50 mL) was placed in 150 mL AceGlass resealable pressure vessel. The white suspension was blanketed with nitrogen. The vessel was sealed and warmed to 115-125° C. for 48 h. The crude reaction was filtered through a short bed of silica gel and washed with ethyl acetate. The filtrate was concentrated in vacuo and purified by silica gel chromatography eluted with ethyl acetate and hexanes (0-50%) to afford the title compound as a colorless foam (566 mg, 73%). ¹H NMR (500 MHz, CHLOROFORM-d) δ 7.95 (d, J=8.2Hz, 2H), 7.22 (d, J=8.2Hz, 2H), 5.31 (d, J=4.6 Hz, 1H), 4.74 (d, J=1.8 Hz, 1H), 4.62 (s, 1H), 3.93 (s, 3H), 3.22-2.99 (m, 9H), 2.79-2.55 (m, 4H), 2.52-2.42 (m, 1H), 2.18-2.09 (m, 1H), 1.99-1.02 (m, 20H), 1.72 (s, 3H), 1.11 (s, 3H), 1.01 (s., 3H), 1.10 (s, 3H), 0.95 (s, 3H), 0.95 (s., 3H). LCMS: m/e 705.51 (M+H)⁺, 3.01 min (method 4).

Intermediate 5 was prepared as shown in the following scheme:

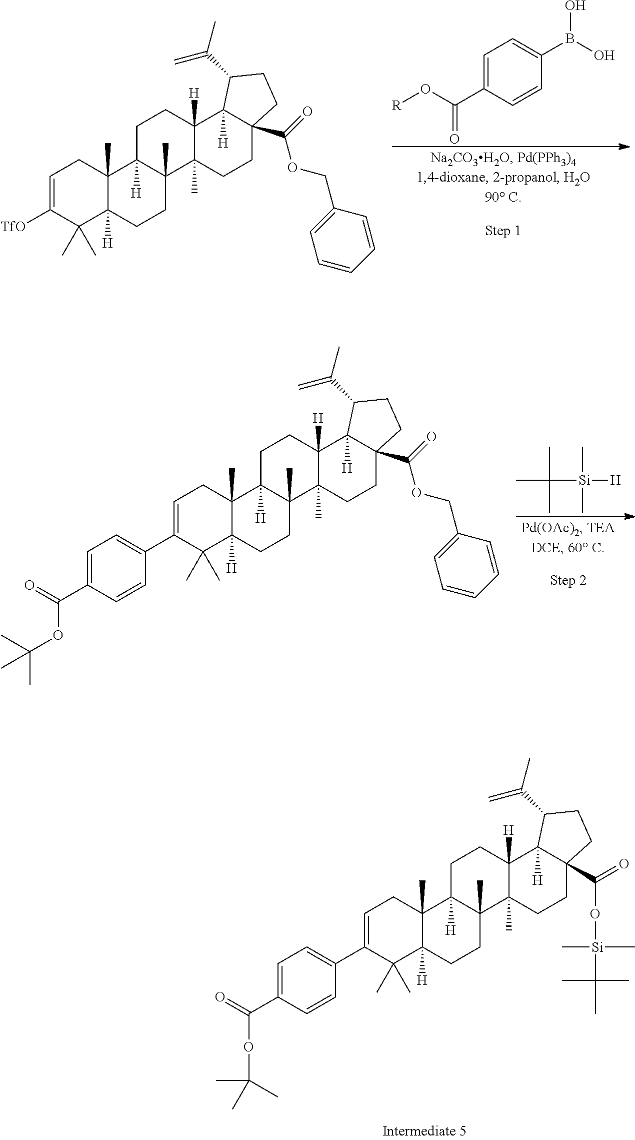

### Step 1: Preparation of (1R,3aS,5aR,5bR,7aR,11aS,11bR,13aR,13bR)-benzyl 9-(4-(tert-butoxycarbonyl)phenyl)-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysene-3a-carboxylate

To a suspension of (1R,3aS,5aR,5bR,7aR,11aR,11bR,13aR,13bR)-benzyl 5a,5b,8,8,11a-pentamethyl-1-(prop-1-en-2-yl)-9-(trifluoromethylsulfonyloxy)-2,3,3a,4,5,5a,5b,6,7,7a,8,11,11a,11b,12,13,13a,13b-octadecahydro-1H-cyclopenta[a]chrysene-3a-carboxylate (17.2 g, 25.4 mmol) in 1,4-dioxane (100 mL) was added 2-propanol (100 mL), water (40 mL), sodium carbonate, monohydrate (9.45 g, 76 mmol), 4-tert-butoxycarbonylphenylboronic acid (8.46 g, 38.1 mmol), and tetrakis(triphenylphosphine)palladium(0) (0.881 g, 0.762 mmol). The flask with the mixture was attached to a reflux condensor, flushed with nitrogen and heated to reflux (90° C. oil bath temp). Upon heating, the solids in the mixture dissolved, and the mixture became a crimson color. After 3.5 h of heating, the mixture was cooled to rt. Upon cooling crystals formed, which were collected by filtration and washed with water. The crystals were dissolved in DCM and EtOH and were concentrated under reduced pressure. The residue was dissolved in DCM and passed through a plug of celite and silica gel. The filtrate was concentrated under reduced pressure to give the expected product, (1R,3aS,5aR,5bR,7aR,11aS,11bR,13aR,13bR)-benzyl 9-(4-(tert-butoxycarbonyl)phenyl)-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysene-3a-carboxylate (13.8 g, 19.57 mmol, 77% yield), as a light gray foam. ¹H NMR (500 MHz, CHLOROFORM-d) δ 7.87 (d, J=8.2Hz, 2H), 7.40-7.29 (m, 5H), 7.16 (d, J=8.2Hz, 2H), 5.26 (dd, J=6.3, 1.7 Hz, 1H), 5.16 (d, J=12.2Hz, 1H), 5.09 (d, J=12.2Hz, 1H), 4.73 (d, J=2.1 Hz, 1H), 4.60 (s, 1H), 3.03 (td, J=10.9, 4.7 Hz, 1H), 2.32-2.20 (m, 2H), 2.08 (dd, J=17.1, 6.4 Hz, 1H), 1.95-1.82 (m, 2H), 1.68 (s, 3H), 1.58 (s, 9H), 0.97 (s, 3H), 0.95 (s, 3H), 0.90 (s, 3H), 0.90 (s, 3H), 1.76-0.88 (m, 17H), 0.82 (s, 3H).

### Step 2: Preparation of (1R,3aS,5aR,5bR,7aR,11aS,11bR,13aR,13bR)-tert-butyldimethylsilyl 9-(4-(tert-butoxycarbonyl)phenyl)-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysene-3a-carboxylate. Intermediate 5

To a solution of (1R,3aS,5aR,5bR,7aR,11aS,11bR,13aR,13bR)-benzyl 9-(4-(tert-butoxycarbonyl)phenyl)-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysene-3a-carboxylate (13.8 g, 19.57 mmol) in DCE (200 mL) was added triethylamine (4.37 mL, 31.3 mmol), tert-butyldimethylsilane (6.49 mL, 39.1 mmol), and palladium(II) acetate (1.099 g, 4.89 mmol). The mixture was flushed with nitrogen and was heated to 60° C. After 3.5 h of heating, the mixture was cooled to rt and was filtered through a pad of silica gel and celite which was washed with dichloromethane followed by 25% ethyl acetate in hexanes. The filtrate was concentrated under reduced pressure. The mixture was diluted with 200 mL of water and was extracted with dichloromethane (3×200 mL). The combined organic layers were dried with sodium sulfate, filtered, and concentrated under reduced pressure to give (1R,3aS,5aR,5bR,7aR,11aS,11bR,13aR,13bR)-tert-butyldimethylsilyl 9-(4-(tert-butoxycarbonyl)phenyl)-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysene-3a-carboxylate (13.75 g, 18.86 mmol, 96% yield) as a white foam. ¹H NMR (500 MHz, Chloroform-d) δ=7.87 (d, J=7.9 Hz, 2H), 7.16 (d, J=7.9 Hz, 2H), 5.26 (d, J=4.9 Hz, 1H), 4.73 (d, J=1.5Hz, 1H), 4.60 (s, 1H), 3.06 (td, J=10.9, 4.7 Hz, 1H), 2.31-2.22 (m, 2H), 2.09 (dd, J=17.2, 6.3 Hz, 1H), 1.98-1.82 (m, 2H), 1.69 (s, 3H), 1.58 (s, 9H), 0.99 (s, 6H), 0.96 (br. s., 3H), 0.96 (s, 9H), 0.91 (s, 6H), 1.76-0.88 (m, 17H), 0.28 (s, 6H).

Intermediate 6 was prepared as shown in the following scheme:

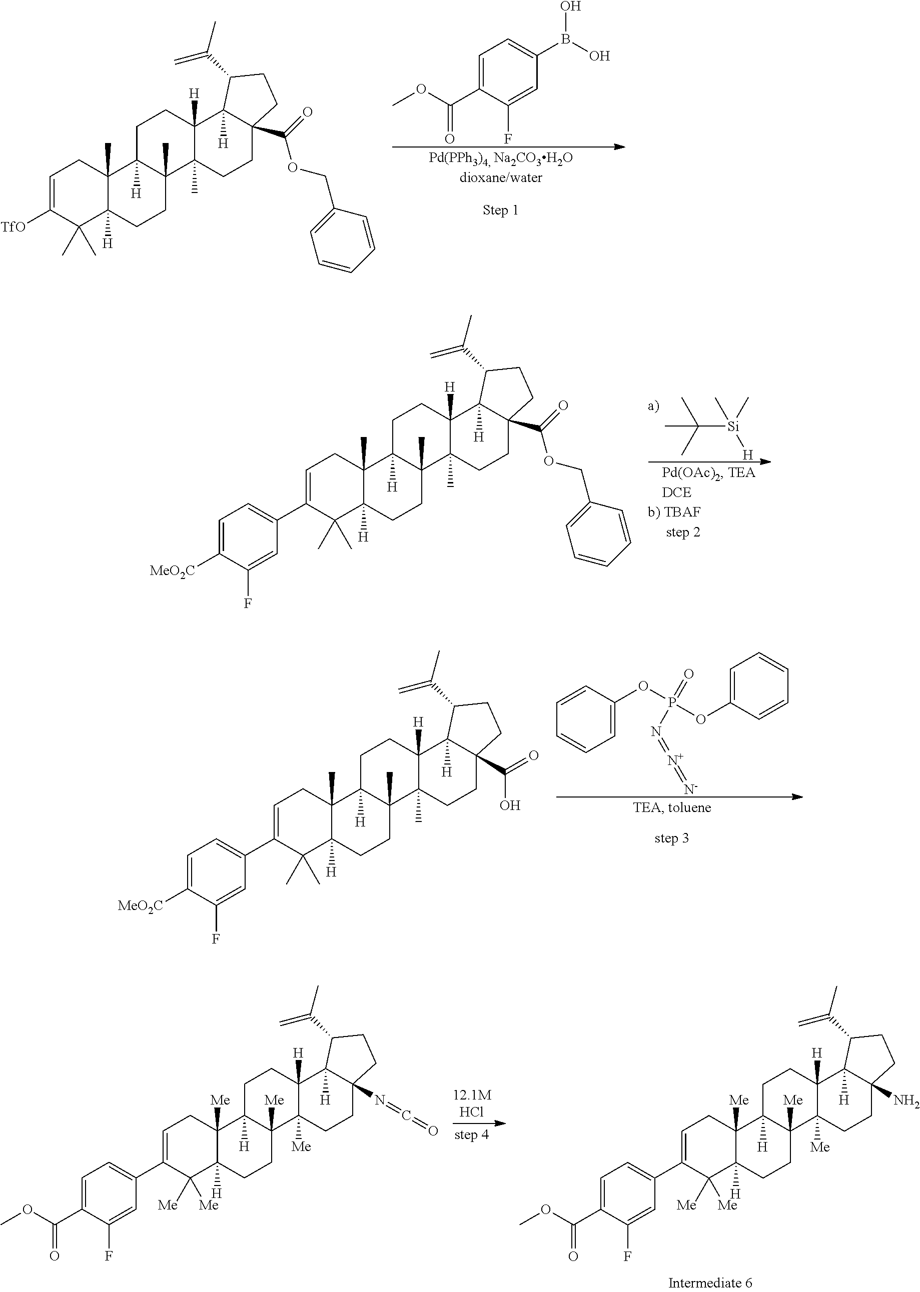

### Step 1. Preparation of (1R,3aS,5aR,5bR,7aR,11aS,11bR,13aR,13bR)-benzyl 9-(3-fluoro-4-(methoxycarbonyl)phenyl)-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysene-3a-carboxylate

A suspension of (1R,3aS,5aR,5bR,7aR,11aR,11bR,13aR,13bR)-benzyl 5a,5b,8,8,11a-pentamethyl-1-(prop-1-en-2-yl)-9-(trifluoromethylsulfonyloxy)-2,3,3a,4,5,5a,5b,6,7,7a,8,11,11a,11b,12,13,13a,13b-octadecahydro-1H-cyclopenta[a]chrysene-3a-carboxylate (4.0 g, 5.91 mmol), 3-fluoro-4-(methoxycarbonyl)phenylboronic acid (1.287 g, 6.50 mmol), sodium carbonate monohydrate (2.198 g, 17.73 mmol), and Pd(PPh₃)₄ (0.205 g, 0.177 mmol) in 1,4-dioxane (24 mL) and water (6 mL) was flushed with N₂ and the mixture was heated to reflux.

After 2 h of heating, the mixture was cooled to rt. The mixture was diluted with water (40 mL) and was extracted with dichloromethane (3×40 mL). The combined organic layers were dried with Na₂SO₄. The drying agent was removed by filtration and the filtrate was concentrated under reduced pressure. The residue was dissolved in DCM and was filtered through a pad of celite and silica gel, washing with a 25% EtOAc in hexanes solution. The filtrate was concentrated under reduced pressure to give the title compound (3.59 g, 5.27 mmol, 89% yield) as a dark grey foam. The crude product was used in the next step with no additional purification. ¹H NMR (500 MHz, CHLOROFORM-d) δ ppm 7.80 (1H, t, J=7.8 Hz), 7.29-7.42 (5H, m), 6.96 (1H, d, J=7.9 Hz), 6.91 (1H, d, J=11.9 Hz), 5.28-5.33 (1H, m), 5.16 (1H, d, J=12.5Hz), 5.09 (1H, d, J=12.2Hz), 4.73 (1H, s), 4.59 (1H, br. s.), 3.92 (3H, s), 3.03 (1H, td, J=10.8, 4.7 Hz), 2.20-2.33 (2H, m), 2.09 (1H, dd, J=17.1, 6.4 Hz), 1.81-1.97 (2H, m), 1.68 (3H, s), 0.96 (3H, s), 0.92 (3H, s), 0.92 (3H, s), 0.91 (3H, s), 0.81 (3H, s), 0.79-1.75 (17H, m).

### Step 2. Preparation of (1R,3aS,5aR,5bR,7aR,11aS,11bR,13aR,13bR)-9-(3-fluoro-4-(methoxycarbonyl)phenyl)-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysene-3a-carboxylic acid

To a solution of (1R,3aS,5aR,5bR,7aR,11aS,11bR,13aR,13bR)-benzyl 9-(3-fluoro-4-(methoxycarbonyl)phenyl)-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysene-3a-carboxylate (3.59 g, 5.27 mmol) in DCE (25 mL) was added TEA (1.176 mL, 8.44 mmol), t-butyldimethylsilane (1.749 mL, 10.54 mmol), and palladium(II) acetate (0.118 g, 0.527 mmol). The mixture was flushed with N₂ and heated to 60° C. for 1 h. The mixture was cooled to rt and was filtered through a plug of celite and silica gel (washed with 25% EtOAc in hexanes). The filtrate was concentrated under reduced pressure. The residue was diluted with 25 mL of dioxane and TBAF (75% in water) (2.76 g, 7.91 mmol) was added. The mixture was stirred for 30 minutes at rt then was diluted with 50 mL of 1N HCl. The solids that formed were collected by filtration and were washed with water to give the title compound (2.95 g, 4.99 mmol, 95% yield) as a white solid. ¹H NMR (400 MHz, CHLOROFORM-d) δ ppm 7.83 (1H, t, J=7.9 Hz), 6.90-7.00 (2H, m), 5.34 (1H, dd, J=6.1, 1.6 Hz), 4.77 (1H, d, J=2.0 Hz), 4.64 (1H, s), 3.94 (3H, s), 3.04 (1H, td, J=10.7, 4.8 Hz), 2.24-2.34 (2H, m), 2.13 (1H, dd, J=17.3, 6.3 Hz), 1.96-2.06 (2H, m), 1.72 (3H, s), 1.03 (3H, s), 1.02 (3H, s), 0.98 (3H, s), 0.93-0.96 (6H, m), 0.91-1.80 (17H, m).

### Step 3. Preparation of methyl 2-fluoro-4-((1R,3aS,5aR,5bR,7aR,11aS,11bR,13aR,13bR)-3a-isocyanato-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysen-9-yl)benzoate

To a solution of (1R,3aS,5aR,5bR,7aR,11aS,11bR,13aR,13bR)-9-(3-fluoro-4-(methoxycarbonyl)phenyl)-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysene-3a-carboxylic acid (2.95 g, 4.99 mmol) in toluene (50 mL) and TEA (1.39 mL, 9.99 mmol) was added diphenyl phosphorazidate (1.614 mL, 7.49 mmol) and the mixture was heated to reflux. After 3 h, the reaction mixture was concentrated under reduced pressure, was adsorbed to silica gel, and was purified by flash chromatography using a 0-10% EtOAc in hexanes gradient to give the title compound as a white solid. The material was used in the next step with no additional purification. ¹H NMR (500 MHz, Chloroform-d) δ ppm 7.81 (1H, t, J=7.8 Hz), 6.96 (1H, dd, J=7.9, 1.5 Hz), 6.92 (1H, dd, J=11.9, 1.5Hz), 5.31 (1H, dd, J=6.3, 1.7 Hz), 4.75 (1H, s), 4.64 (1H, s), 3.92 (3H, s), 2.55 (1H, td, J=10.8, 5.8 Hz), 2.05-2.16 (2H, m), 1.76-1.92 (4H, m), 1.68 (3H, s), 1.09-1.11 (3H, m), 0.97 (3H, s), 0.96 (3H, s), 0.94 (3H, s), 0.92 (3H, s), 0.88-1.75 (16H, m).

### Step 4. Preparation of methyl 4-((1R,3aS,5aR,5bR,7aR,11aS,11bR,13aR,13bR)-3a-amino-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysen-9-yl)-2-fluorobenzoate dihydrochloride. Intermediate 6

To a solution of crude methyl 2-fluoro-4-((1R,3aS,5aR,5bR,7aR,11aS,11bR,13aR,13bR)-3a-isocyanato-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysen-9-yl)benzoate (2.93 g, 4.99 mmol) in THF (35 mL) was added 12N HCl (10 mL, 121 mmol). After stirring the mixture for 24 h, the mixture was diluted with water (100 mL) until solids precipitated. The solids were collected by filtration and were washed with water to afford the title compound (2.75 g, 4.33 mmol, 87% yield), as an off-white solid that was used in the next step with no additional purification. LCMS: m/e 562 (M+H)⁺, 1.96 min (method 5).

General scheme for the preparation of C-30 amines (Examples 1-6).

Example 1-6 were prepared either from intermediate 2 or 5 following the scheme below:

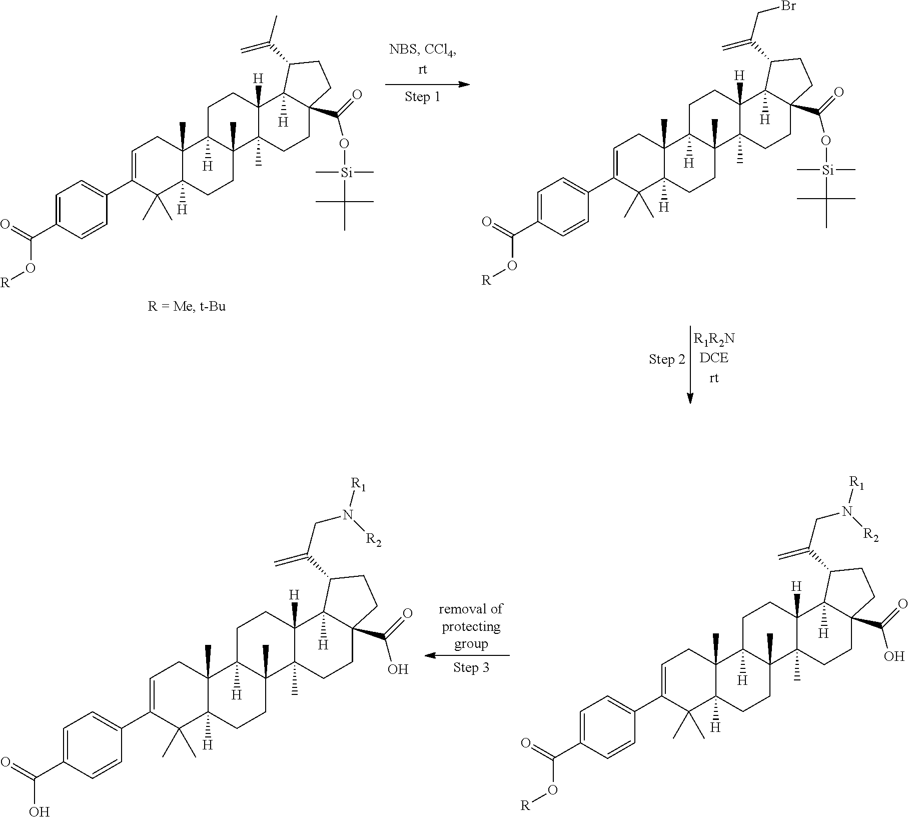

### Example 1

### Preparation of (1R,3aS,5aR,5bR,7aR,11aS,11bR,13aR,13bS)-9-(4-carboxyphenyl)-5a,5b,8,8,11a-pentamethyl-1-(3-(2-morpholinoethylamino)prop-1-en-2-yl)-2,3,3a,4,5,5a,5b,6,7,7a,8,11,11a,11b,12,13,13a,13b-octadecahydro-1H-cyclopenta[a]chrysene-3a-carboxylic acid

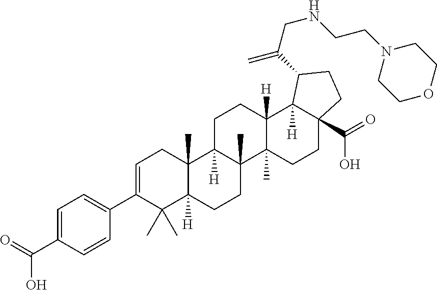

### Step 1: Preparation of (1R,3aS,5aR,5bR,7aR,11aS,11bR,13aR,13bS)-tert-butyldimethylsilyl 1-(3-bromoprop-1-en-2-yl)-9-(4-(methoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylate

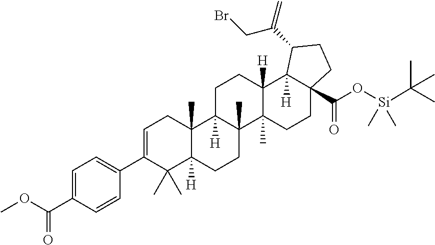

To a solution of (1R,3aS,5aR,5bR,7aR,11aS,11bR,13aR,13bR)-tert-butyldimethylsilyl 9-(4-(methoxycarbonyl)phenyl)-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysene-3a-carboxylate (0.315 g, 0.458 mmol), in CCl₄ (5 mL) was added NBS (0.102 g, 0.573 mmol). The mixture was stirred at rt for 16 h then was filtered through a pad of celite (washed with DCM) and the filtrate was concentrated under reduced pressure. The residue was loaded onto a 12 g silica gel column and was purified by flash chromatography using a 0-10% ethyl acetate in hexanes gradient. The fractions containing the expected product were combined and concentrated under reduced pressure to give (1R,3aS,5aR,5bR,7aR,11aS,11bR,13aR,13bS)-tert-butyldimethylsilyl 1-(3-bromoprop-1-en-2-yl)-9-(4-(methoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylate (0.205 g, 0.268 mmol, 58.4% yield), as a white foam. LCMS: m/e 765, 767.5 (M+H)⁺, 4.78 min (method 1). ¹H NMR (500 MHz, chloroform-d) δ 7.91 (d, J=8.2Hz, 2H), 7.18 (d, J=8.2Hz, 2H), 5.30-5.25 (m, 1H), 5.13 (s, 1H), 5.04 (s, 1H), 4.01-3.96 (m, 2H), 3.89 (s, 3H), 3.09 (td, J=11.2, 4.1 Hz, 1H), 2.31-2.24 (m, 2H), 2.17-2.05 (m, 2H), 1.86 (dd, J=12.4, 7.8 Hz, 1H), 0.96 (s, 9H), 1.80-0.76 (m, 32H), 0.31-0.27 (m, 6H).

### Step 2: Preparation of (1R,3aS,5aR,5bR,7aR,11aS,11bR,13aR,13bS)-9-(4-(methoxycarbonyl)phenyl)-5a,5b,8,8,11a-pentamethyl-1-(3-(2-morpholinoethylamino)prop-1-en-2-yl)-2,3,3a,4,5,5a,5b,6,7,7a,8,11,11a,11b,12,13,13a,13b-octadecahydro-1H-cyclopenta[a]chrysene-3a-carboxylic acid

To a flask containing (1R,3aS,5aR,5bR,7aR,11aS,11bR,13aR,13bS)-tert-butyldimethylsilyl 1-(3-bromoprop-1-en-2-yl)-9-(4-(methoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylate (26 mg, 0.034 mmol) was added 2-morpholinoethylamine (0.056 mL, 0.430 mmol). The slurry formed was dissolved in DCE (1 mL) and was stirred at rt overnight. After 18.5 h of stirring the mixture at rt, it was concentrated under a stream of nitrogen and was purified by prep HPLC (method 1). The fractions containing the product were combined and concentrated under reduced pressure to give (1R,3aS,5aR,5bR,7aR,11aS,11bR,13aR,13bS)-9-(4-(methoxycarbonyl)phenyl)-5a,5b,8,8,11a-pentamethyl-1-(3-(2-morpholinoethylamino)prop-1-en-2-yl)-2,3,3a,4,5,5a,5b,6,7,7a,8,11,11a,11b,12,13,13a,13b-octadecahydro-1H-cyclopenta[a]chrysene-3a-carboxylic acid (17.6 mg, 0.025 mmol, 74.0% yield) as a white foam. LCMS: m/e 701.6 (M+H)⁺, 2.37 min (method 1). ¹H NMR (500 MHz, chloroform-d) δ ppm 7.91 (d, J=8.24 Hz, 2H), 7.17 (d, J=8.24 Hz, 2H), 5.27 (d, J=4.58 Hz, 1H), 5.06 (s, 1H), 4.97 (s, 1H), 3.90 (s, 3H), 3.73 (t, J=4.27 Hz, 3H), 3.40-3.50 (m, 2H), 2.86-3.00 (m, 3H), 2.59-2.72 (m, 2H), 2.53 (br. s., 4H), 2.24-2.37 (m, 2H), 2.05-2.16 (m, 2H), 1.88-1.96 (m, 1H), 1.61-1.75 (m, 2H), 1.03-1.57 (m, 16H), 1.00 (s, 3H), 0.97 (s, 3H), 0.96 (s, 3H), 0.91 (s, 6H).

### Step 3: Protecting Group Removal

To a solution of (1R,3aS,5aR,5bR,7aR,11aS,11bR,13aR,13bS)-9-(4-(methoxycarbonyl)phenyl)-5a,5b,8,8,11a-pentamethyl-1-(3-(2-morpholinoethylamino)prop-1-en-2-yl)-2,3,3a,4,5,5a,5b,6,7,7a,8,11,11a,11b,12,13,13a,13b-octadecahydro-1H-cyclopenta[a]chrysene-3a-carboxylic acid (17.6 mg, 0.025 mmol) in 1,4-dioxane (1 mL) was added 1N NaOH (0.126 mL, 0.126 mmol). The mixture was heated to 75° C. for 19.5 h then was cooled to rt. To the mixture was added 5 mL of 1N HCl and the mixture was concentrated under reduced pressure. The residue was purified by prep HPLC (method 1) to give (1R,3aS,5aR,5bR,7aR,11aS,11bR,13aR,13bS)-9-(4-carboxyphenyl)-5a,5b,8,8,11a-pentamethyl-1-(3-(2-morpholinoethylamino)prop-1-en-2-yl)-2,3,3a,4,5,5a,5b,6,7,7a,8,11,11a,11b,12,13,13a,13b-octadecahydro-1H-cyclopenta[a]chrysene-3a-carboxylic acid (6.4 mg, 9.32 mmol, 37.1% yield) as an off-white solid. LCMS: m/e 687.5 (M+H)⁺, 1.99 min (method 1). ¹H NMR (500 MHz, acetic acid d₄) δ ppm 8.03 (d, J=8.24 Hz, 2H), 7.30 (d, J=8.55Hz, 2H), 5.38 (d, J=4.88 Hz, 1H), 5.23 (s, 1H), 5.07 (s, 1H), 3.96 (br. s., 4H), 3.69-3.90 (m, 6H), 3.45 (br. s., 4H), 2.96-3.07 (m, 1H), 2.30-2.40 (m, 2H), 1.10 (s, 3H), 1.08-2.24 (m, 20H), 1.07 (s, 3H), 1.06 (s, 3H), 1.01 (s, 3H), 1.00 (s, 3H).

### Example 2

### Preparation of (1R,3aS,5aR,5bR,7aR,11aS,11bR,13aR,13bS)-9-(4-carboxyphenyl)-1-(3-(2-hydroxyethylamino)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid

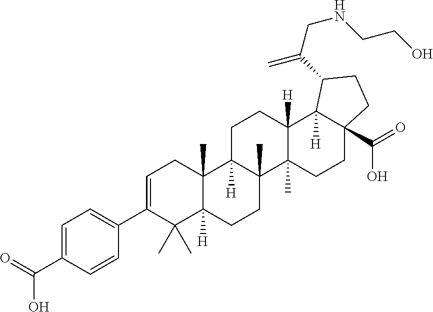

### Step 1: Preparation of (1R,3aS,5aR,5bR,7aR,11aS,11bR,13aR,13bS)-tert-butyldimethylsilyl 1-(3-bromoprop-1-en-2-yl)-9-(4-(tert-butoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylate

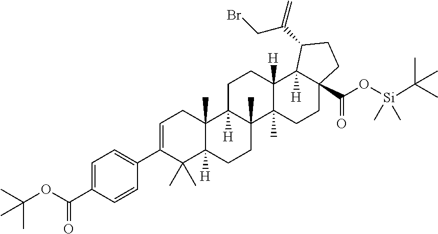

To a solution of (1R,3aS,5aR,5bR,7aR,11aS,11bR,13aR,13bR)-tert-butyldimethylsilyl 9-(4-(tert-butoxycarbonyl)phenyl)-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysene-3a-carboxylate (2.0 g, 2.74 mmol) in CCl₄ (10 mL) was added NBS (0.57 g, 3.20 mmol) portionwise over 1 h. The mixture was stirred at rt for 6 h. The mixture was filtered through a pad of celite (washed with DCM) and was concentrated under reduced pressure. The residue was loaded onto a 90 g silica gel column and was purified by flash chromatography using a 0-10% ethyl acetate in hexanes gradient. The fractions containing the expected product were combined and concentrated under reduced pressure to give the title compound (1.29 g, 1.60 mmol, 58.4% yield) as a white foam. LCMS: m/e 807, 809.4 (M+H)⁺, 6.31 min (method 1).

### Step 2: Preparation of (1R,3aS,5aR,5bR,7aR,11aS,11bR,13aR,13bS)-9-(4-(tert-butoxycarbonyl)phenyl)-1-(3-(2-hydroxyethylamino)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid

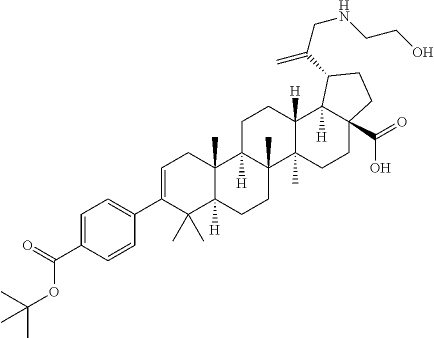

To a solution of (1R,3aS,5aR,5bR,7aR,11aS,11bR,13aR,13bS)-tert-butyldimethylsilyl 1-(3-bromoprop-1-en-2-yl)-9-(4-(tert-butoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylate (0.125 g, 0.155 mmol) in DCE (2 mL) was added ethanolamine (0.093 mL, 1.547 mmol). The mixture was stirred at rt for 20.5 h then was concentrated under reduced pressure and was purified by prep HPLC (method 1). The fractions containing the product were combined and concentrated under reduced pressure to give the title compound (56 mg, 0.083 mmol, 53.7% yield) as a white foam. LCMS: m/e 674.4 (M+H)⁺, 2.45 min (method 1). ¹H NMR (500 MHz, chloroform-d) δ 7.87 (d, J=8.2Hz, 2H), 7.15 (d, J=8.2Hz, 2H), 5.26 (d, J=4.9 Hz, 1H), 5.15 (s, 1H), 5.08 (s, 1H), 3.93-3.80 (m, 2H), 3.50 (br. s., 2H), 3.07-2.90 (m, 3H), 1.58 (s, 9H), 0.99 (s, 6H), 0.97 (s, 3H), 0.90 (s, 6H), 2.43-0.84 (m, 22H).

### Step 3: Removal of Protecting Group

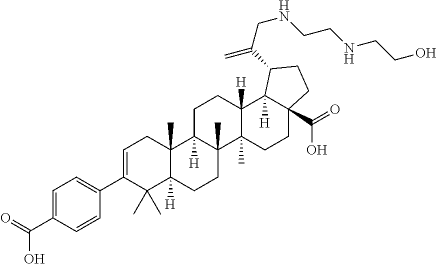

To a solution of (1R,3aS,5aR,5bR,7aR,11aS,11bR,13aR,13bS)-9-(4-(tert-butoxycarbonyl)phenyl)-1-(3-(2-hydroxyethylamino)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid (0.025 g, 0.037 mmol) in DCM (0.5 mL) was added TFA (0.1 mL, 1.298 mmol). The mixture was stirred at rt for 16.25 h then was concentrated under a stream of nitrogen. The residue was purified by prep HPLC (method 1). The fractions containing the expected product were combined and concentrated under reduced pressure to give (1R,3aS,5aR,5bR,7aR,11aS,11bR,13aR,13bS)-9-(4-carboxyphenyl)-1-(3-(2-hydroxyethylamino)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid (8.0 mg, 0.013 mmol, 34.9% yield) as a white solid. LCMS: m/e 618.3 (M+H)⁺, 2.00 min (method 1). ¹H NMR (400 MHz, acetic acid-d4) δ ppm 8.03 (d, J=8.03 Hz, 2H), 7.29 (d, J=8.03 Hz, 2H), 5.37 (d, J=5.27 Hz, 1H), 5.25 (s, 1H), 5.18 (s, 1H), 4.02 (t, J=4.77 Hz, 2H), 3.75-3.92 (m, 2H), 3.37-3.44 (m, 2H), 2.98-3.07 (m, 1H), 2.29-2.42 (m, 2H), 1.11-2.24 (m, 20H), 1.10 (s, 3H), 1.06 (s, 6H), 1.00 (s, 3H), 0.99 (s, 3H).

### Example 3

### Preparation of (1R,3aS,5aR,5bR,7aR,11aS,11bR,13aR,13bS)-9-(4-carboxyphenyl)-1-(3-(dimethylamino)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid

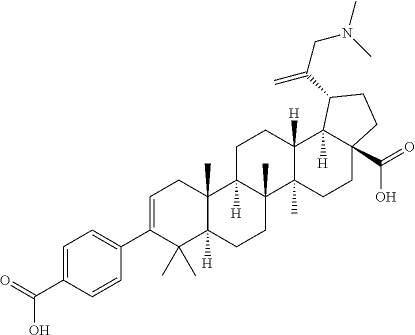

### Step 2: Preparation of (1R,3aS,5aR,5bR,7aR,11aS,11bR,13aR,13bS)-9-(4-(tert-butoxycarbonyl)phenyl)-1-(3-(dimethylamino)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid

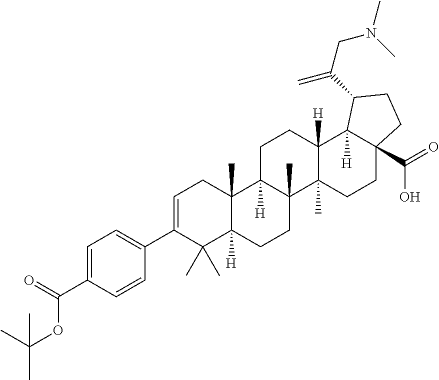

To a solution of (1R,3aS,5aR,5bR,7aR,11aS,11bR,13aR,13bS)-tert-butyldimethylsilyl 1-(3-bromoprop-1-en-2-yl)-9-(4-(tert-butoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylate (0.125 g, 0.155 mmol) in DCE (1 mL) was added dimethylamine (2M in THF) (0.773 mL, 1.547 mmol). After 21 h of stirring the mixture at rt, it was concentrated under reduced pressure and was purified by prep HPLC (method 1). The fractions containing the product were combined and concentrated under reduced pressure to give (1R,3aS,5aR,5bR,7aR,11aS,11bR,13aR,13bS)-9-(4-(tert-butoxycarbonyl)phenyl)-1-(3-(dimethylamino)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid (74 mg, 0.112 mmol, 72.7% yield) as a white foam. LCMS: m/e 658.6 (M+H)⁺, 2.75 min (method 1).

Step 3: To a solution of (1R,3aS,5aR,5bR,7aR,11aS,11bR,13aR,13bS)-9-(4-(tert-butoxycarbonyl)phenyl)-1-(3-(dimethylamino)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid (25 mg, 0.038 mmol) in DCM (0.5 mL) was added TFA (0.1 mL, 1.298 mmol). The mixture was stirred at rt for 16.5 h then was concentrated under a stream of nitrogen. The residue was dissolved in methanol and dioxane and was purified by prep HPLC. The fractions containing the expected product were combined and concentrated under reduced pressure to give (1R,3aS,5aR,5bR,7aR,11aS,11bR,13aR,13bS)-9-(4-carboxyphenyl)-1-(3-(dimethylamino)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid (5.8 mg, 9.64 mmol, 25.4% yield) as a white solid. LCMS: m/e 602.4 (M+H)⁺, 2.05 min (method 1). ¹H NMR (400 MHz, acetic acid-d₄) δ ppm 7.99 (d, J=8.28 Hz, 2H), 7.25 (d, J=8.28 Hz, 2H), 5.38 (s, 1H), 5.33 (d, J=4.77 Hz, 1H), 5.26 (s, 1H), 3.89 (d, J=14.05Hz, 1H), 3.73 (d, J=14.31 Hz, 1H), 2.93-3.03 (m, 1H), 2.93 (s, 6H), 0.99-2.40 (m, 22H),1.05 (s, 3H), 1.03 (s, 6H), 0.97 (s, 3H), 0.95 (s, 3H).

### Example 4

### Preparation of (1R,3aS,5aR,5bR,7aR,11aS,11bR,13aR,13bS)-9-(4-carboxyphenyl)-1-(3-((2-(dimethylamino)ethyl)(methyl)amino)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid

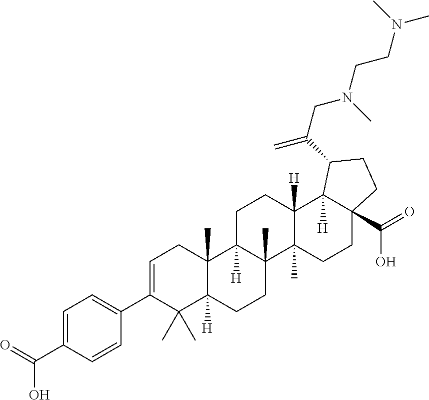

### Step 2: Preparation of (1R,3aS,5aR,5bR,7aR,11aS,11bR,13aR,13bS)-9-(4-(tert-butoxycarbonyl)phenyl)-1-(3-(2-(dimethylamino)ethyl)(methyl)amino)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid

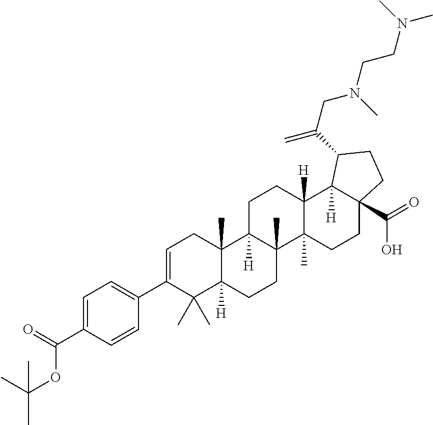

To a solution of (1R,3aS,5aR,5bR,7aR,11aS,11bR,13aR,13bS)-tert-butyldimethylsilyl 1-(3-bromoprop-1-en-2-yl)-9-(4-(tert-butoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylate (0.125 g, 0.155 mmol) in DCE (2 mL) was added N,N,N′-Trimethylethylenediamine (0.201 mL, 1.547 mmol). The mixture was stirred at rt for 20 h then was concentrated under reduced pressure and was purified by prep HPLC (method 1). The fractions containing the product were combined and concentrated under reduced pressure to give (1R,3aS,5aR,5bR,7aR,11aS,11bR,13aR,13bS)-9-(4-(tert-butoxycarbonyl)phenyl)-1-(3-((2-(dimethylamino)ethyl)(methyl)amino)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid (64 mg, 0.090 mmol, 57.9% yield) as a white foam. LCMS: m/e 713.5 (M−H)⁻, 2.76 min (method 1).

Step 3:

To a solution of (1R,3aS,5aR,5bR,7aR,11aS,11bR,13aR,13bS)-9-(4-(tert-butoxycarbonyl)phenyl)-1-(3-((2-(dimethylamino)ethyl)(methyl)amino)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid (0.025 g, 0.035 mmol) in DCM (0.5 mL) was added TFA (0.1 mL, 1.298 mmol). The mixture was stirred at rt for 16.5 h, then was concentrated under a stream of nitrogen. The residue was dissolved in methanol and dioxane and was purified by prep HPLC (method 1). The fractions containing the expected product were combined and concentrated under reduced pressure to give (1R,3aS,5aR,5bR,7aR,11aS,11bR,13aR,13bS)-9-(4-carboxyphenyl)-1-(3-[2-(dimethylamino)ethyl)(methyl)amino)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid (17.8 mg, 0.027 mmol, 77% yield) as a white solid. LCMS: m/e 657.4 (M−H)⁻, 2.16 min (method 1). ¹H NMR (400 MHz, acetic acid-d₄) δ ppm 7.99 (d, J=8.28 Hz, 2H), 7.25 (d, J=8.28 Hz, 2H), 5.42 (s, 1H), 5.31-5.35 (m, 1H), 5.31 (s, 1H), 3.83-4.00 (m, 2H), 3.77 (s, 4H), 2.88-3.00 (m, 10H), 2.25-2.39 (m, 2H), 0.99-2.24 (m, 20H), 1.05 (s, 3H), 1.03 (s, 6H), 0.97 (s, 3H), 0.95 (s, 3H).

### Example 5

### Preparation of (1R,3aS,5aR,5bR,7aR,11aS,11bR,13aR,13bS)-9-(4-carboxyphenyl)-1-(3-(2-(dimethylamino)ethylamino)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid

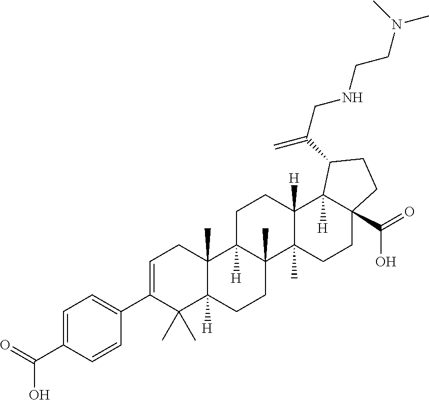

### Step 2: Preparation of (1R,3aS,5aR,5bR,7aR,11aS,11bR,13aR,13bS)-9-(4-(tert-butoxycarbonyl)phenyl)-1-(3-(2-(dimethylamino)ethylamino)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid

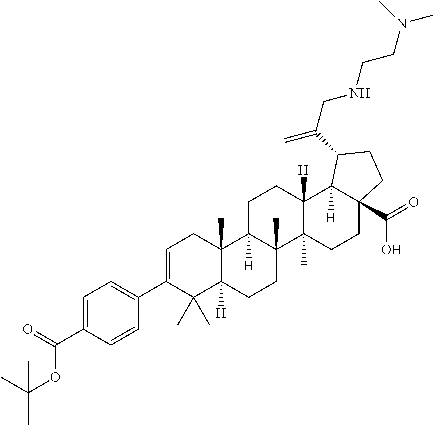

To a solution of (1R,3aS,5aR,5bR,7aR,11aS,11bR,13aR,13bS)-tert-butyldimethylsilyl 1-(3-bromoprop-1-en-2-yl)-9-(4-(tert-butoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylate (0.125 g, 0.155 mmol) in DCE (2 mL) was added N,N-dimethylethylenediamine. The mixture was stirred at rt for 21 h then was concentrated under reduced pressure and was purified by prep HPLC (method 1). The fractions containing the product were combined and concentrated under reduced pressure to give (1R,3aS,5aR,5bR,7aR,11aS,11bR,13aR,13bS)-9-(4-(tert-butoxycarbonyl)phenyl)-1-(3-(2-(dimethylamino)ethylamino)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid (78 mg, 0.111 mmol, 71.9% yield) as a white foam. LCMS: m/e 699.5 (M−H)⁻, 2.53 min (method 1).

Step 3: To a solution of (1R,3aS,5aR,5bR,7aR,11aS,11bR,13aR,13bS)-9-(4-(tert-butoxycarbonyl)phenyl)-1-(3-(2-(dimethylamino)ethylamino)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid (25 mg, 0.036 mmol) in DCM (0.5 mL) was added TFA (0.1 mL, 1.298 mmol). The mixture was stirred at rt for 15.5 h then was concentrated under a stream of nitrogen and the residue was purified by prep HPLC (method 1). The fractions containing the expected product were combined and concentrated under reduced pressure. The product, (1R,3aS,5aR,5bR,7aR,11aS,11bR,13aR,13bS)-9-(4-carboxyphenyl)-1-(3-(2-(dimethylamino)ethylamino)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid (4.7 mg, 7.29 μmol, 20% yield), was isolated as a white solid. LCMS: m/e 643.4 (M−H)⁻, 2.08 min (method 1). ¹H NMR (500 MHz, acetic acid) δ ppm 8.03 (d, J=8.24 Hz, 2H), 7.30 (d, J=8.24 Hz, 2H), 5.38 (d, J=4.88 Hz, 1H), 5.23 (s, 1H), 5.10 (s, 1H), 3.70-3.90 (m, 6H), 2.99 (s, 6H), 2.97-3.08 (m, 1H), 2.30-2.40 (m, 2H), 1.08-2.24 (m, 20H), 1.10 (s, 3H), 1.07 (s, 6H), 1.01 (s, 3H), 1.00 (s, 3H).

### Example 6

### Preparation of (1R,3aS,5aR,5bR,7aR,11aS,11bR,13aR,13bS)-1-(3-(2-carboxyethylamino)prop-1-en-2-yl)-9-(4-carboxyphenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid

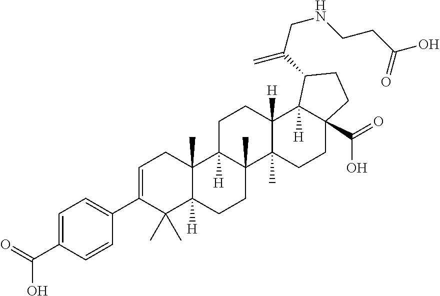

### Step 2: Preparation of (1R,3aS,5aR,5bR,7aR,11aS,11bR,13aR,13bS)-9-(4-(tert-butoxycarbonyl)phenyl)-1-(3-(3-ethoxy-3-oxopropylamino)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid

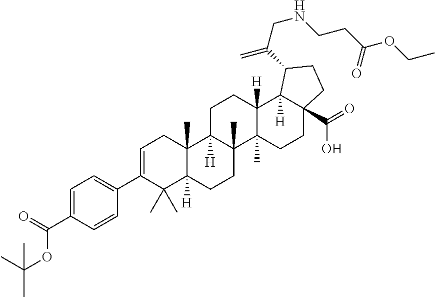

To a solution of (1R,3aS,5aR,5bR,7aR,11aS,11bR,13aR,13bS)-tert-butyldimethylsilyl 1-(3-bromoprop-1-en-2-yl)-9-(4-(tert-butoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylate (0.125 g, 0.155 mmol) in DCE (2 mL) and triethylamine (0.216 mL, 1.547 mmol) was added beta-alanine, ethyl ester hydrochloride (0.238 g, 1.547 mmol). The mixture was stirred at rt for 22 h, then was warmed to 40° C. and was stirred foran additional 6 h. The mixture was cooled to rt and was stirred for 90 h at rt then was concentrated under reduced pressure. The residue was purified by prep HPLC (method 1). The fractions containing the expected product were combined and concentrated under reduced pressure to give (1R,3aS,5aR,5bR,7aR,11aS,11bR,13aR,13bS)-9-(4-(tert-butoxycarbonyl)phenyl)-1-(3-(3-ethoxy-3-oxopropylamino)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid (73 mg, 0.100 mmol, 64.6% yield) as an off-white solid. LCMS: m/e 728.5 (M−H)⁻, 2.57 min (method 1).

Step 3: To a solution of (1R,3aS,5aR,5bR,7aR,11aS,11bR,13aR,13bS)-9-(4-(tert-butoxycarbonyl)phenyl)-1-(3-(3-ethoxy-3-oxopropylamino)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid (0.035 g, 0.048 mmol) in DCM (0.5 mL) was added TFA (0.1 mL, 1.298 mmol). The mixture was stirred at rt for 21.5 h then the solvent was removed under a stream of nitrogen. The crude product was dissolved in 0.5 mL of dioxane and 0.4 mL of 1N NaOH was added to the mixture. It was warmed to 75° C. 18.25 h then was cooled to rt. The mixture was diluted with 1 mL of methanol and was purified by prep HPLC (method 1). The fractions containing the expected product were combined and concentrated under reduced pressure to give(1R,3aS,5aR,5bR,7aR,11aS,11bR,13aR,13bS)-1-(3-(2-carboxyethylamino)prop-1-en-2-yl)-9-(4-carboxyphenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid (11 mg, 0.017 mmol, 35.5% yield) as a white solid. LCMS: m/e 644.4 (M−H)⁻, 1.91 min (method 1). ¹H NMR (500 MHz, Acetic Acid-d₄) δ ppm 8.03 (d, J=8.24 Hz, 2H), 7.30 (d, J=8.24 Hz, 2H), 5.37 (d, J=5.19 Hz, 1H), 5.25 (s, 1H), 5.19 (s, 1H), 3.86-3.91 (m, 1H), 3.78-3.83 (m, 1H), 3.50 (t, J=6.71 Hz, 2H), 2.98-3.07 (m, 1H), 2.94 (t, J=6.71 Hz, 2H), 2.30-2.40 (m, 2H), 2.12-2.23 (m, 2H), 1.10-2.10 (m, 18H), 1.10 (s, 3H), 1.07 (s, 6H), 1.01 (s, 3H), 1.00 (s, 3H).

### Example 7

### Preparation of 4-((1R,3aS,5aR,5bR,7aR,11aS,11bR,13aR,13bS)-3a-(2-(dimethylamino)ethylcarbamoyl)-1-(3-(dimethylamino)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid

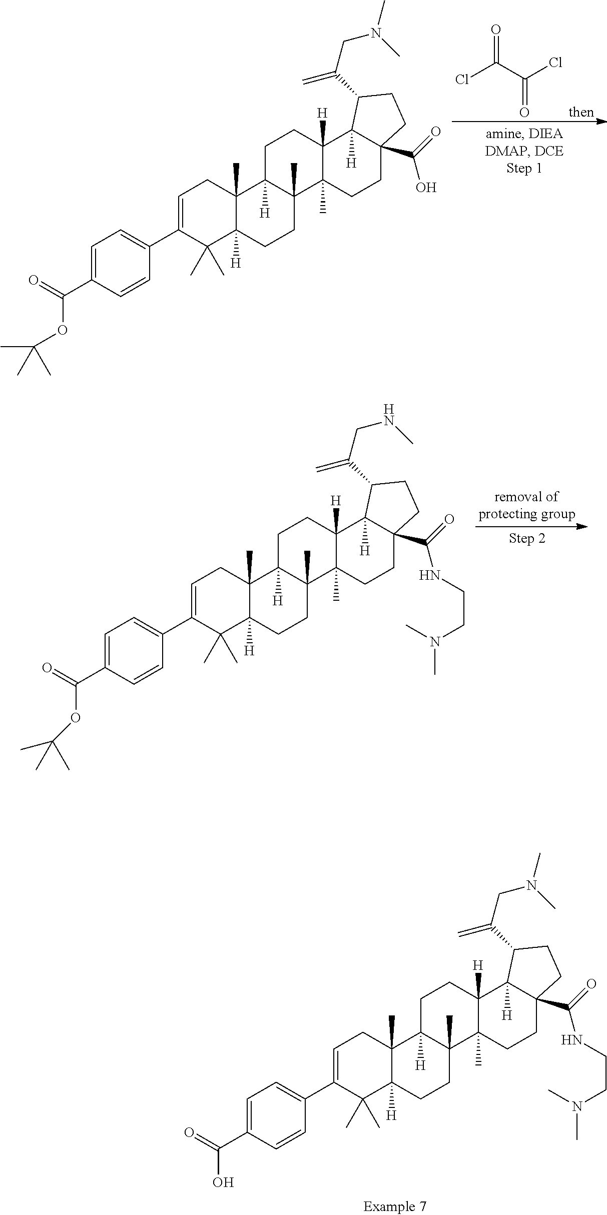

### Step 1: Preparation of tert-butyl 4-((1R,3aS,5aR,5bR,7aR,11aS,11bR,13aR,13bS)-3a-(2-(dimethylamino)ethylcarbamoyl)-1-(3-(dimethylamino)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate

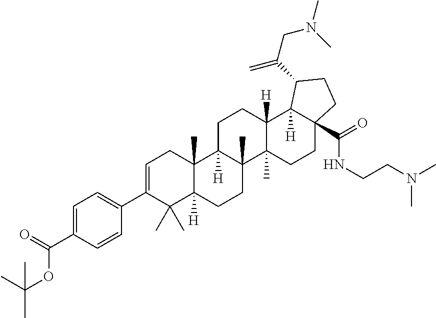

To a vial containing (1R,3aS,5aR,5bR,7aR,11aS,11bR,13aR,13bS)-9-(4-(tert-butoxycarbonyl)phenyl)-1-(3-(dimethylamino)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid (50 mg, 0.076 mmol) was added oxalyl chloride (2M in DCM) (2 mL, 4.00 mmol). The mixture was stirred at rt for 2 h then was concentrated under reduced pressure. The residue was dissolved in DCM and concentrated under reduced pressure two additional times. The crude product was diluted with DCE (2 mL) and DIEA (0.066 mL, 0.380 mmol), N,N-dimethylethylenediamine (0.022 mL, 0.204 mmol), and DMAP (1 mg, 8.19 mmol) were added. The mixture was stirred at rt for 18.5 h then was concentrated under reduced pressure. The crude product was used in the next step with no purification. LCMS: m/e 726.6 (M−H)⁻, 2.87 min (method 1).

Step 2: To a solution of tert-butyl 4-((1R,3aS,5aR,5bR,7aR,11aS,11bR,13aR,13bS)-3a-(2-(dimethylamino)ethylcarbamoyl)-1-(3-(dimethylamino)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (55.3 mg, 0.076 mmol) in DCM (1 mL) was added TFA (0.1 mL, 1.298 mmol). The mixture was stirred at rt for 15 h then was concentrated under a stream of nitrogen and the residue was purified by prep HPLC (method 1). The fractions containing the expected product were combined and concentrated under reduced pressure. The expected product, 4-((1R,3aS,5aR,5bR,7aR,11aS,11bR,13aR,13bS)-3a-(2-(dimethylamino)ethylcarbamoyl)-1-(3-(dimethylamino)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid (24.8 mg, 0.037 mmol, 48.6% yield), was isolated as a white solid. LCMS: m/e 672.4 (M+H)⁺, 2.10 min (method 1). ¹H NMR (500 MHz, acetic acid-d₄) δ ppm 8.03 (d, J=8.24 Hz, 2H), 7.29 (d, J=8.24 Hz, 2H), 5.40 (s, 1H), 5.37 (d, J=5.19 Hz, 1H), 5.29 (s, 1H), 3.91 (d, J=14.34 Hz, 1H), 3.68-3.80 (m, 3H), 3.33-3.42 (m, 2H), 3.09-3.17 (m, 1H), 2.99 (s, 6H), 2.96 (s, 6H), 2.50-2.57 (m, 1H), 1.08-2.23 (m, 21H), 1.08 (s, 3H), 1.07 (s, 3H), 1.04 (s, 3H), 1.01 (s, 3H), 0.99 (s, 3H).

### Example 8

### Preparation of 4-((1R,3aS,5aR,5bR,7aR,11aS,11bR,13aR,13bS)-1-(3-((2-(dimethylamino)ethyl)(methyl)amino)prop-1-en-2-yl)-3a-(2-(dimethylamino)ethylcarbamoyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid

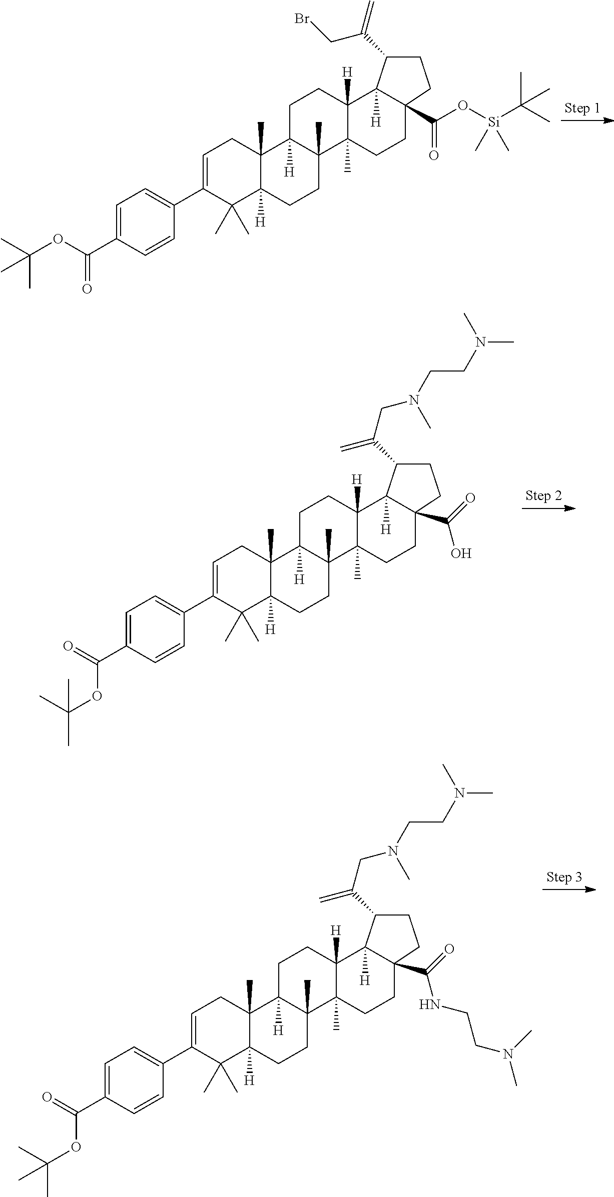

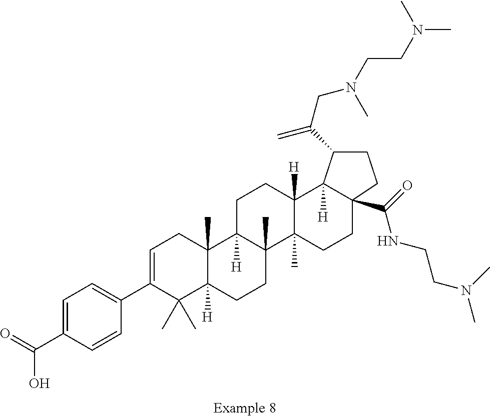

### Step 1: Preparation of (1R,3aS,5aR,5bR,7aR,11aS,11bR,13aR,13bS)-9-(4-(tert-butoxycarbonyl)phenyl)-1-(3-(2-(dimethylamino)ethyl)(methyl)amino)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid

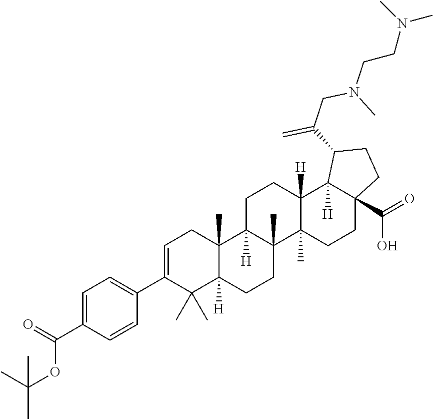

To a solution of (1R,3aS,5aR,5bR,7aR,11aS,11bR,13aR,13bS)-tert-butyldimethylsilyl 1-(3-bromoprop-1-en-2-yl)-9-(4-(tert-butoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylate (0.125 g, 0.155 mmol) in DCE (2 mL) was added N,N,N′-Trimethylethylenediamine (0.201 mL, 1.547 mmol). The mixture was stirred at rt for 20 h then was concentrated under reduced pressure and was purified by prep HPLC (method 1). The fractions containing the product were combined and concentrated under reduced pressure to give (1R,3aS,5aR,5bR,7aR,11aS,11bR,13aR,13bS)-9-(4-(tert-butoxycarbonyl)phenyl)-1-(3-((2-(dimethylamino)ethyl)(methyl)amino)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid (64 mg, 0.090 mmol, 57.9% yield) as a white foam. LCMS: m/e 713.5 (M−H)⁻, 2.76 min (method 1).

### Step 2: Preparation of tert-butyl 4-((1R,3aS,5aR,5bR,7aR,11aS,11bR,13aR,13bS)-1-(3-((2-(dimethylamino)ethyl)(methyl)amino)prop-1-en-2-yl)-3a-(2-(dimethylamino)ethylcarbamoyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate

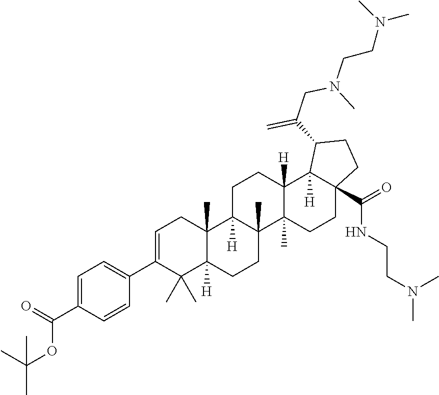

To a vial containing (1R,3aS,5aR,5bR,7aR,11aS,11bR,13aR,13bS)-9-(4-(tert-butoxycarbonyl)phenyl)-1-(3-((2-(dimethylamino)ethyl)(methyl)amino)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid (0.037 g, 0.052 mmol) was added oxalyl chloride (2M in DCM) (2 mL, 4.00 mmol). The mixture was stirred at rt for 2.5 hours and was concentrated under reduced pressure. The residue was dissolved in DCM and concentrated two additional times. The crude residue was diluted with DCE (2 mL) and DIEA (0.045 mL, 0.259 mmol). N,N-Dimethylaminoethylamine (0.011 mL, 0.103 mmol) and DMAP (1 mg, 8.19 μmol) were added and the mixture was stirred for 18.5 h then was concentrated under reduced pressure to give the crude product which was used directly in the next step with no additional purification. LCMS: m/e 786.65 (M+H)⁺, 2.77 min (method 1).

Step 3: To a solution of tert-butyl 4-((1R,3aS,5aR,5bR,7aR,11aS,11bR,13aR,13bS)-1-(3-((2-(dimethylamino)ethyl)(methyl)amino)prop-1-en-2-yl)-3a-(2-(dimethylamino)ethylcarbamoyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (40.8 mg, 0.052 mmol) in DCM (1 mL) was added TFA (0.1 mL, 1.298 mmol). The mixture was stirred at rt for 15 h then was concentrated under a stream of nitrogen and the residue was purified by prep HPLC (method 1). The fractions containing the expected product were combined and concentrated under reduced pressure to give 4-((1R,3aS,5aR,5bR,7aR,11aS,11bR,13aR,13bS)-1-(3-((2-(dimethylamino)ethyl)(methyl)amino)prop-1-en-2-yl)-3a-(2-(dimethylamino)ethylcarbamoyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid (27 mg, 0.037 mmol, 71.2% yield) as a white solid. LCMS: m/e 729.64 (M+H)⁺, 2.14 min (method 1). ¹H NMR (500 MHz, acetic acid-d₄) δ ppm 8.03 (d, J=7.93 Hz, 2H), 7.29 (d, J=8.24 Hz, 2H), 5.43 (s, 1H), 5.37 (d, J=4.88 Hz, 1H), 5.31 (s, 1H), 4.00 (d, J=14.34 Hz, 1H), 3.88 (d, J=14.34 Hz, 1H), 3.82 (br. s., 4H), 3.73 (t, J=5.65Hz, 2H), 3.33-3.42 (m, 2H), 3.08-3.18 (m, 1H), 3.01 (s, 3H), 2.98 (s, 6H), 2.95 (s, 6H), 2.51-2.58 (m, 1H), 2.13-2.23 (m, 2H), 1.09-2.13 (m, 19H), 1.08 (s, 3H), 1.07 (s, 3H), 1.04 (s, 3H), 1.01 (s, 3H), 1.00 (s, 3H).

### Example 9

### Preparation of 4-((1R,3aS,5aR,5bR,7aR,11aS,11bR,13aR,13bS)-3a-(2-carboxyethylcarbamoyl)-1-(3-(2-(dimethylamino)ethylamino)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid

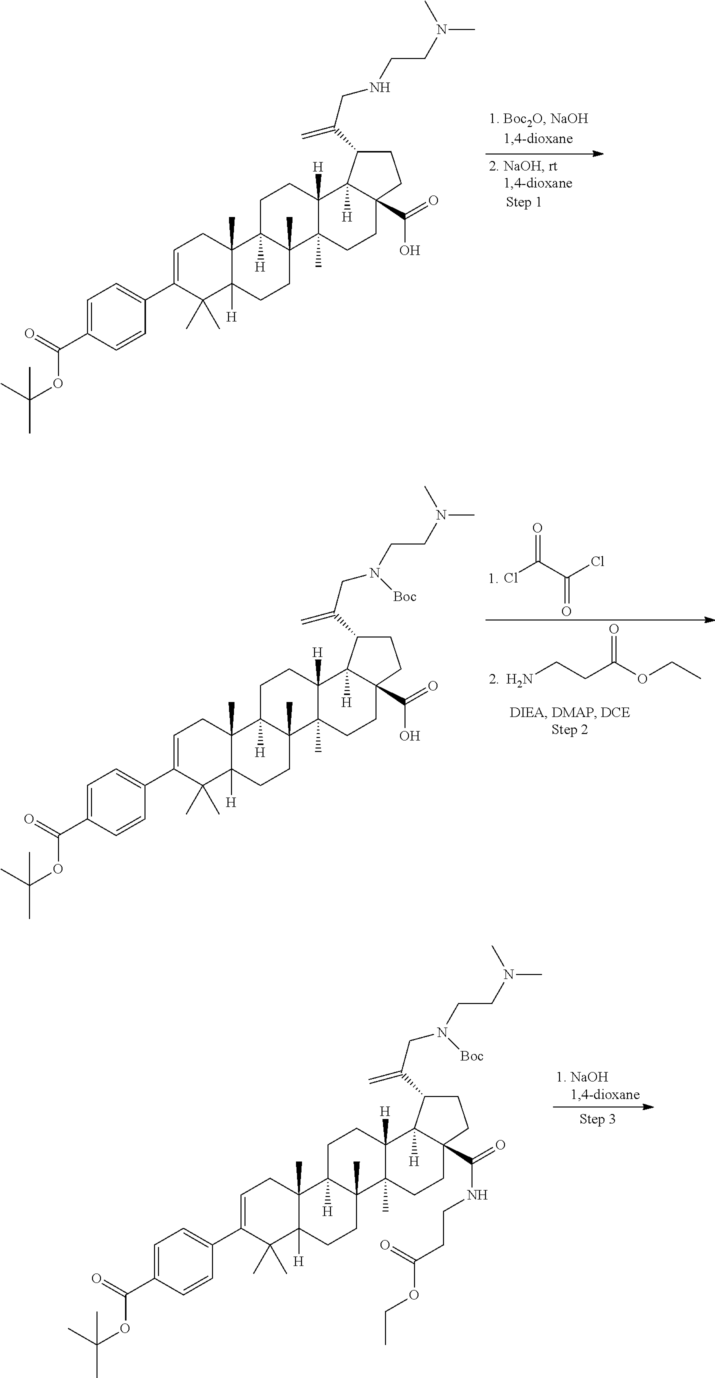

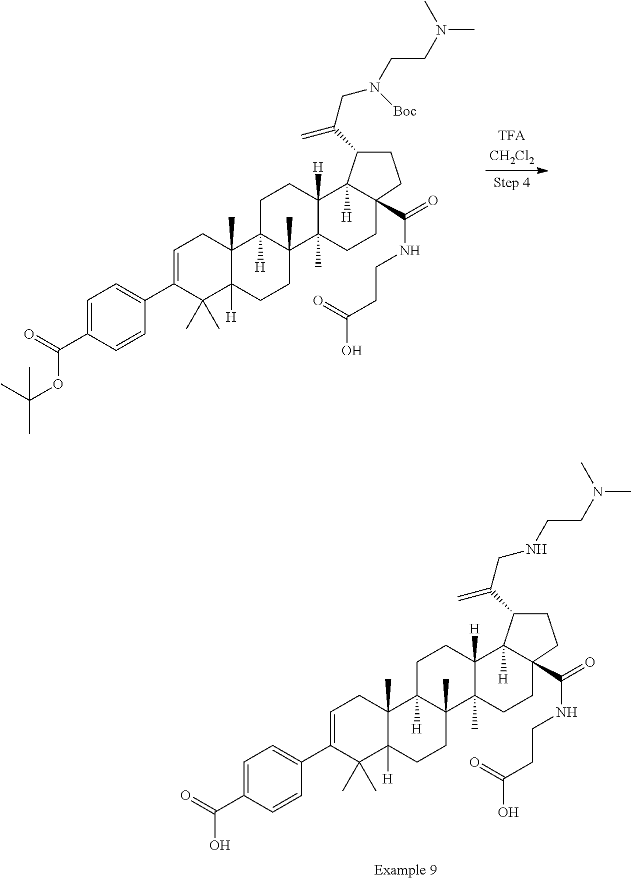

### Step 1: Preparation of (1R,3aS,5aR,5bR,7aR,11aS,11bR,13aR,13bS)-1-(3-(tert-butoxycarbonyl(2-(dimethylamino)ethyl)amino)prop-1-en-2-yl)-9-(4-(tert-butoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid

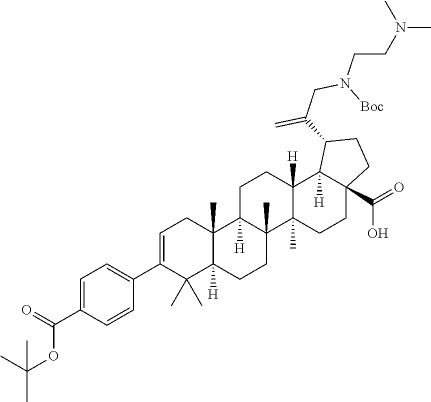

To a solution of (1R,3aS,5aR,5bR,7aR,11aS,11bR,13aR,13bS)-9-(4-(tert-butoxycarbonyl)phenyl)-1-(3-(2-(dimethylamino)ethylamino)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid (45 mg, 0.064 mmol) in 1,4-dioxane (2 mL) was added sodium hydroxide (1N) (0.25 mL, 0.250 mmol) and Boc₂O (0.030 mL, 0.128 mmol). The mixture was stirred at rt for 17.5 h then was diluted with water (4 mL) and extracted with dichloromethane (3×5 mL). The combined organic layers were dried with sodium sulfate, filtered and concentrated under reduced pressure. The residue was dissolved in 1,4-dioxane (1 mL) and was treated with 1N sodium hydroxide (0.2 mL, 0.2 mmol). The mixture was stirred for 97 h then was diluted with methanol and purified by prep HPLC. The fractions containing the product were combined and concentrated under reduced pressure to give (1R,3aS,5aR,5bR,7aR,11aS,11bR,13aR,13bS)-1-(3-(tert-butoxycarbonyl(2-(dimethylamino)ethyl)amino)prop-1-en-2-yl)-9-(4-(tert-butoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid as a white solid. LCMS: m/e 799.6 (M−H)⁻, 2.76 min (method 1).

### Step 2: Preparation of tert-butyl 4-((1R,3aS,5aR,5bR,7aR,11aS,11bR,13aR,13bS)-1-(3-(tert-butoxycarbonyl(2-(dimethylamino)ethyl)amino)prop-1-en-2-yl)-3a-(3-ethoxy-3-oxopropylcarbamoyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate

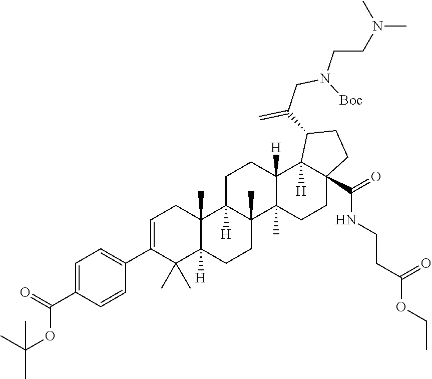

To a flask containing (1R,3aS,5aR,5bR,7aR,11aS,11bR,13aR,13bS)-1-(3-(tert-butoxycarbonyl(2-(dimethylamino)ethyl)amino)prop-1-en-2-yl)-9-(4-(tert-butoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid (26 mg, 0.024 mmol) was added oxalyl chloride (2M in DCM) (1 mL, 2.000 mmol). The mixture was stirred at rt for two hours then was concentrated under reduced pressure. The residue was dissolved in DCM and was concentrated two additional times. The crude product was dissolved in DCE (1 mL) and DIEA (0.021 mL, 0.122 mmol), beta-Alanine, ethyl ester hydrochloride (7.48 mg, 0.049 mmol), and DMAP (0.5 mg, 4.09 μmol) were added. The mixture was stirred at rt for 2.5 h then the reaction was quenched with water (5 mL) and extracted with dichloromethane (3×5 mL). The combined organic layers were dried with sodium sulfate, filtered, and concentrated under reduced pressure to give the crude product which was used in the next step with no additional purification. LCMS: m/e 898.7 (M−H)⁻, 2.75 min (method 1).

### Step 3: Preparation of 3-((1R,3aS,5aR,5bR,7aR,11aS,11bR,13aR,13bS)-1-(3-(tert-butoxycarbonyl(2-(dimethylamino)ethyl)amino)prop-1-en-2-yl)-9-(4-(tert-butoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxamido)propanoic acid

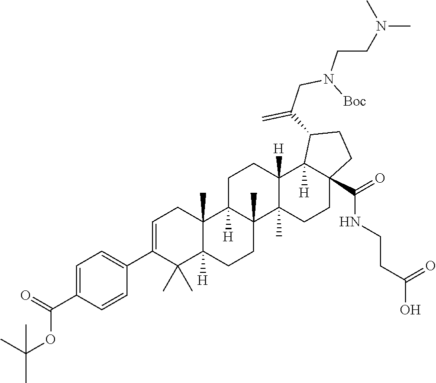

To a solution of tert-butyl 4-((1R,3aS,5aR,5bR,7aR,11aS,11bR,13aR,13bS)-1-(3-(tert-butoxycarbonyl(2-(dimethylamino)ethyl)amino)prop-1-en-2-yl)-3a-(3-ethoxy-3-oxopropylcarbamoyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (0.022 g, 0.024 mmol) in 1,4-dioxane (1 mL) was added NaOH (1N) (0.120 mL, 0.120 mmol). The mixture was heated to 75° C. for 87 h, then was cooled to rt and concentrated under reduced pressure. The crude product was used in the next step with no additional purification. LCMS: m/e 870.6 (M−H)⁻, 2.46 min (method 1).

### Step 4: BOC Deprotection

To a solution of 3-((1R,3aS,5aR,5bR,7aR,11aS,11bR,13aR,13bS)-1-(3-(tert-butoxycarbonyl(2-(dimethylamino)ethyl)amino)prop-1-en-2-yl)-9-(4-(tert-butoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxamido)propanoic acid (20.93 mg, 0.024 mmol) in dichloromethane (2 mL) was added TFA (0.25 mL, 3.24 mmol). The mixture was stirred at rt for 16.5 h then was concentrated under a stream of nitrogen. The residue was dissolved in dioxane and methanol and was purified by prep HPLC (method 1). The fractions containing the expected product were combined and concentrated under reduced pressure. The residue which contained impurities was repurified by prep HPLC (method 9). The fractions containing the product were combined and concentrated under reduced pressure to give 4-((1R,3aS,5aR,5bR,7aR,11aS,11bR,13aR,13bS)-3a-(2-carboxyethylcarbamoyl)-1-(3-(2-(dimethylamino)ethylamino)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid (6.2 mg, 8.66 μmol, 13.5% yield over 4 steps) as a white solid. LCMS: m/e 714.5 (M−H)⁻, 1.93 min (method 1). ¹H NMR (500 MHz, acetic acid d₄) δ ppm 8.03 (d, J=8.24 Hz, 2H), 7.30 (d, J=8.24 Hz, 2H), 5.37 (d, J=4.88 Hz, 1H), 5.21 (s, 1H), 5.05 (s, 1H), 3.69-3.90 (m, 6H), 3.45-3.67 (m, 2H), 3.19 (t, J=12.51 Hz, 1H), 2.99 (s, 6H), 2.68 (t, J=6.41 Hz, 2H), 2.51-2.63 (m, 1H), 1.08-2.25 (m, 21H), 1.08 (s, 3H), 1.07 (s, 3H), 1.06 (s, 3H), 1.01 (s, 3H), 0.99 (s, 3H).

### Example 10

### Preparation of 4-((1R,3aS,5aR,5bR,7aR,11aS,11bR,13aR,13bS)-1-(3-(3-carboxy-N-methylpropanamido)prop-1-en-2-yl)-3a-(2-carboxyethylcarbamoyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid

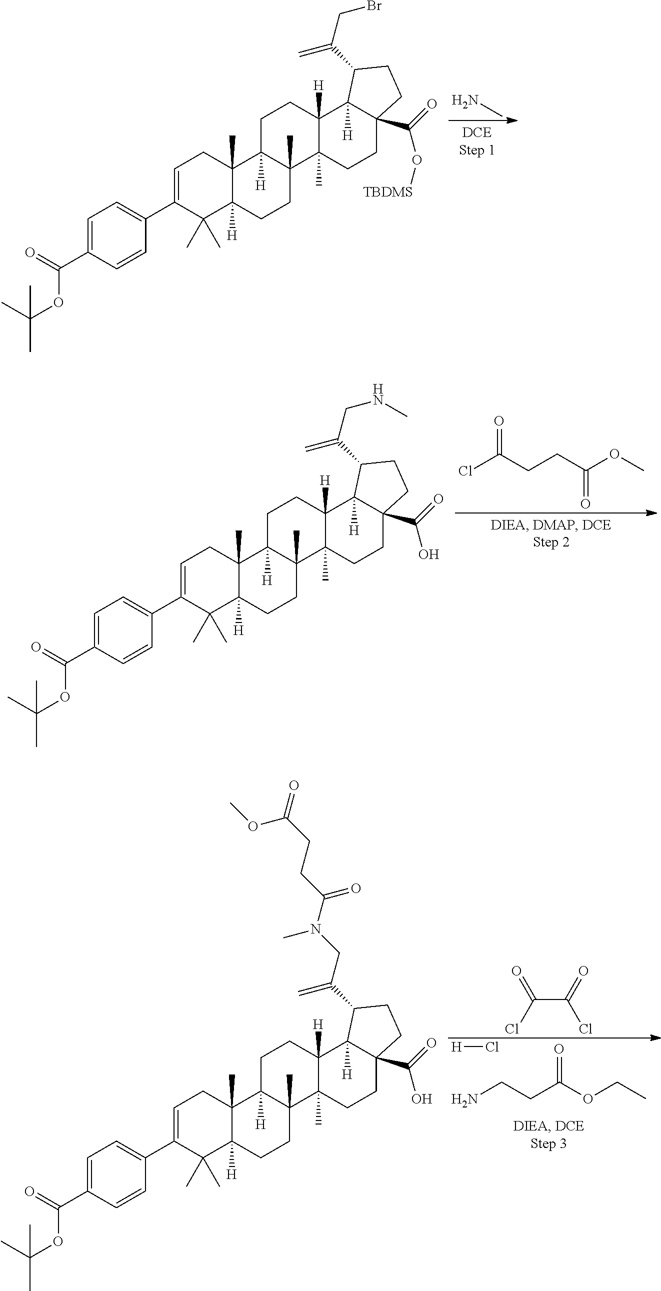

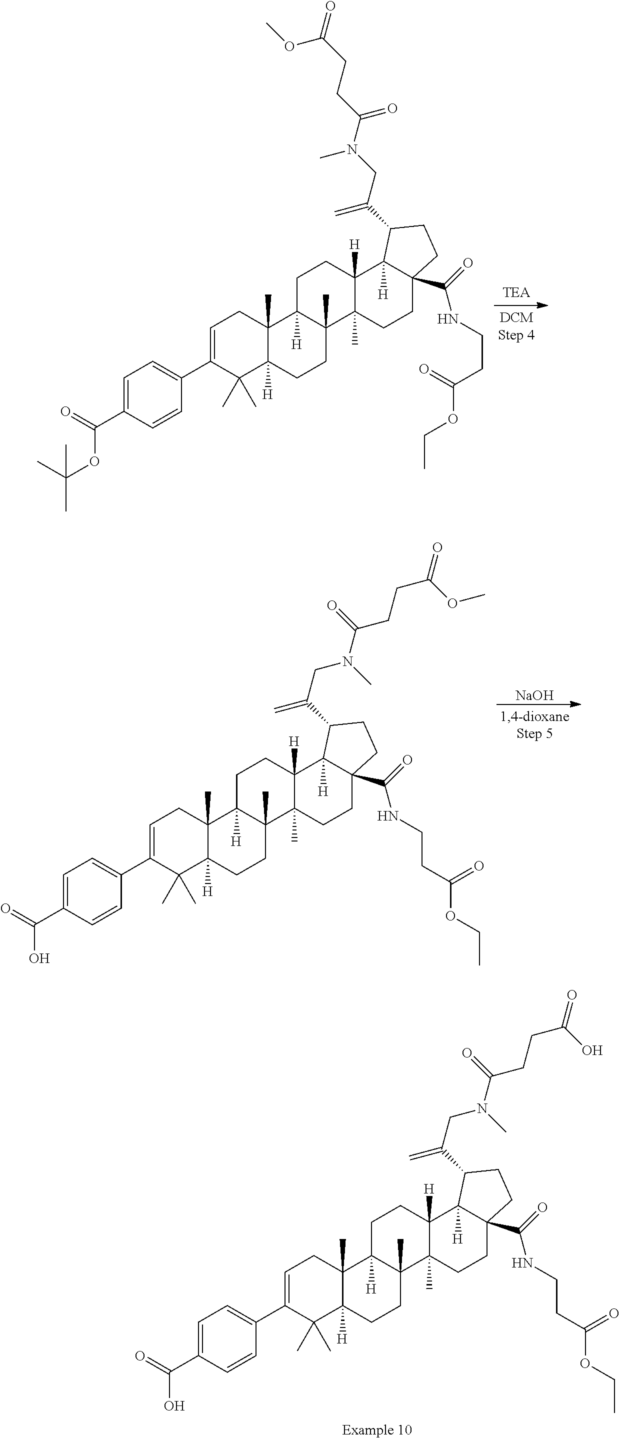

### Step 1: Preparation of (1R,3aS,5aR,5bR,7aR,11aS,11bR,13aR,13bS)-9-(4-(tert-butoxycarbonyl)phenyl)-5a,5b,8,8,11a-pentamethyl-1-(3-(methylamino)prop-1-en-2-yl)-2,3,3a,4,5,5a,5b,6,7,7a,8,11,11a,11b,12,13,13a,13b-octadecahydro-1H-cyclopenta[a]chrysene-3a-carboxylic acid

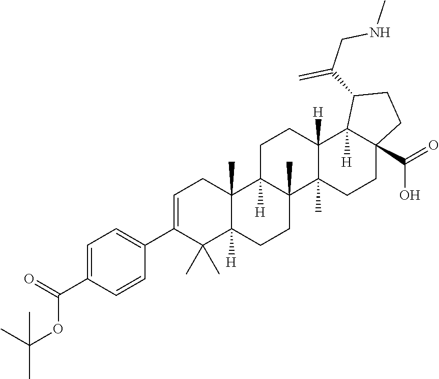

To a solution of (1R,3aS,5aR,5bR,7aR,11aS,11bR,13aR,13bS)-tert-butyldimethylsilyl 1-(3-bromoprop-1-en-2-yl)-9-(4-(tert-butoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylate (0.125 g, 0.155 mmol) in DCE (1 mL) was added methylamine (2M in THF) (0.773 mL, 1.547 mmol). The mixture was stirred at rt for 20 h then an additional 0.8 mL of the methylamine (2M in THF) was added and the mixture was stirred at rt for 1 h, then was warmed to 40° C. and was stirred for and additional 8 h. The mixture was concentrated under reduced pressure and the product was crystallized from a solution of methanol, 1,4-dioxane, and water to give the crude product as an off-white solid (85 mg, 0.132 mmol, 85% yield). LC/MS: m/e 644.4 (M+H)⁺, 2.44 minutes (method 1).

### Step 2: Preparation of (1R,3aS,5aR,5bR,7aR,11aS,11bR,13aR,13bS)-9-(4-(tert-butoxycarbonyl)phenyl)-1-(3-(4-methoxy-N-methyl-4-oxobutanamido)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid

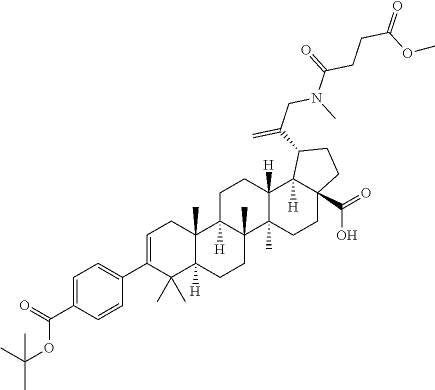

To a solution of (1R,3aS,5aR,5bR,7aR,11aS,11bR,13aR,13bS)-9-(4-(tert-butoxycarbonyl)phenyl)-5a,5b,8,8,11a-pentamethyl-1-(3-(methylamino)prop-1-en-2-yl)-2,3,3a,4,5,5a,5b,6,7,7a,8,11,11a,11b,12,13,13a,13b-octadecahydro-1H-cyclopenta[a]chrysene-3a-carboxylic acid (0.04 g, 0.062 mmol) in DCE (2 mL) was added DIEA (0.054 mL, 0.311 mmol), methyl 4-chloro-4-oxobutyrate (0.038 mL, 0.311 mmol), and DMAP (1 mg, 8.19 μmol). The mixture was stirred at rt for 3 h then was diluted with 2 mL of water and 6 mL of 1N HCl, and was extracted with dichloromethane (3×7 mL). The combined organic layers were dried with sodium sulfate, filtered, and concentrated under reduced pressure. The residue was purified by flash chromatography using a 0-100% ethyl acetate in hexanes gradient with 0.1% acetic acid added to the mixture. The fractions containing the expected product were combined and concentrated under reduced pressure to give the expected product as a white solid (0.047 g, 0.062 mmol, 50% yield). LC/MS: m/e 758.4 (M+H)⁺, 2.57 minutes (method 1).

### Step 3: Preparation of tert-butyl 4-((1R,3aS,5aR,5bR,7aR,11aS,11bR,13aR,13bS)-3a-(3-ethoxy-3-oxopropylcarbamoyl)-1-(3-(4-methoxy-N-methyl-4-oxobutanamido)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate

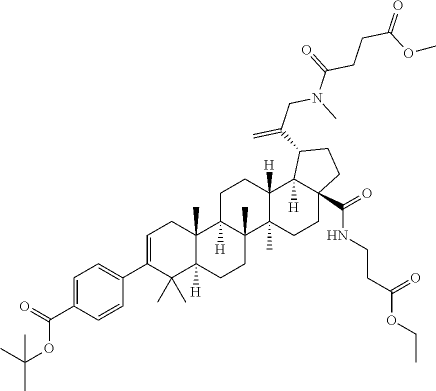

To a vial containing (1R,3aS,5aR,5bR,7aR,11aS,11bR,13aR,13bS)-9-(4-(tert-butoxycarbonyl)phenyl)-1-(3-(4-methoxy-N-methyl-4-oxobutanamido)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid (0.047 g, 0.062 mmol) was added oxalyl chloride (2M in dichloromethane) (1 mL, 2.0 mmol). The solution was stirred at rt for 2 h, then was concentrated under reduced pressure. The residue was dissolved in dichloromethane and concentrated two additional times, then was dried under house vacuum for 1 h. The residue was dissolved in DCE (1 mL) and Hunig's Base (0.032 mL, 0.186 mmol) was added followed by beta-Alanine, ethyl ester hydrochloride (0.014 g, 0.093 mmol). The mixture was stirred at rt for 17 h then was concentrated under a stream of nitrogen and was purified by flash chromatography using a 0-50% ethyl acetate in hexanes gradient and a 12 g silica gel column. The fractions containing the expected product were combined and concentrated under reduced pressure. The expected product was isolated as a white solid (15 mg, 0.017 mmol, 28% yield). LC/MS: m/e 857.5 (M+H)⁺, 2.57 minutes (method 1).

### Step 4: Preparation of 4-((1R,3aS,5aR,5bR,7aR,11aS,11bR,13aR,13bS)-3a-(3-ethoxy-3-oxopropylcarbamoyl)-1-(3-(4-methoxy-N-methyl-4-oxobutanamido)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid

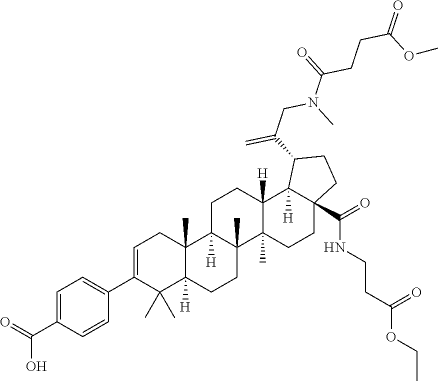

To a solution of tert-butyl 4-((1R,3aS,5aR,5bR,7aR,11aS,11bR,13aR,13bS)-3a-(3-ethoxy-3-oxopropylcarbamoyl)-1-(3-(4-methoxy-N-methyl-4-oxobutanamido)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (0.015 g, 0.017 mmol) in dichloromethane (1 mL) was added TFA (0.05 mL, 0.649 mmol). The mixture was stirred at rt for 23 h then was concentrated under reduced pressure and was used in the next step with no additional purification. LC/MS: m/e 801.4 (M+H)⁺, 2.11 minutes (method 1).

Step 5: To a solution of 4-((1R,3aS,5aR,5bR,7aR,11aS,11bR,13aR,13bS)-3a-(3-ethoxy-3-oxopropylcarbamoyl)-1-(3-(4-methoxy-N-methyl-4-oxobutanamido)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid (0.017 mmol) in 1,4-dioxane (1 mL) was added sodium hydroxide (1N) (0.1 mL, 0.100 mmol). The mixture was heated to 75° C. for 72 h then was purified by prep HPLC. The fractions containing the expected product were combined and concentrated under reduced pressure. The residue was dissolved in acetic acid and concentrated under reduced pressure to give 4-((1R,3aS,5aR,5bR,7aR,11aS,11bR,13aR,13bS)-1-(3-(3-carboxy-N-methylpropanamido)prop-1-en-2-yl)-3a-(2-carboxyethylcarbamoyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid (3.0 mg, 0.0037 mmol, 22% yield) as a clear, colorless film. LC/MS: m/e 759.4 (M+H)⁺, 1.74 minutes (method 1).

### Example 11

### Preparation of (1R,3aS,5aR,5bR,7aR,11aS,11bR,13aR,13bS)-9-(4-carboxyphenyl)-5a,5b,8,8,11a-pentamethyl-1-(3-(2-morpholinoethoxy)prop-1-en-2-yl)-2,3,3a,4,5,5a,5b,6,7,7a,8,11,11a,11b,12,13,13a,13b-octadecahydro-1H-cyclopenta[a]chrysene-3a-carboxylic acid

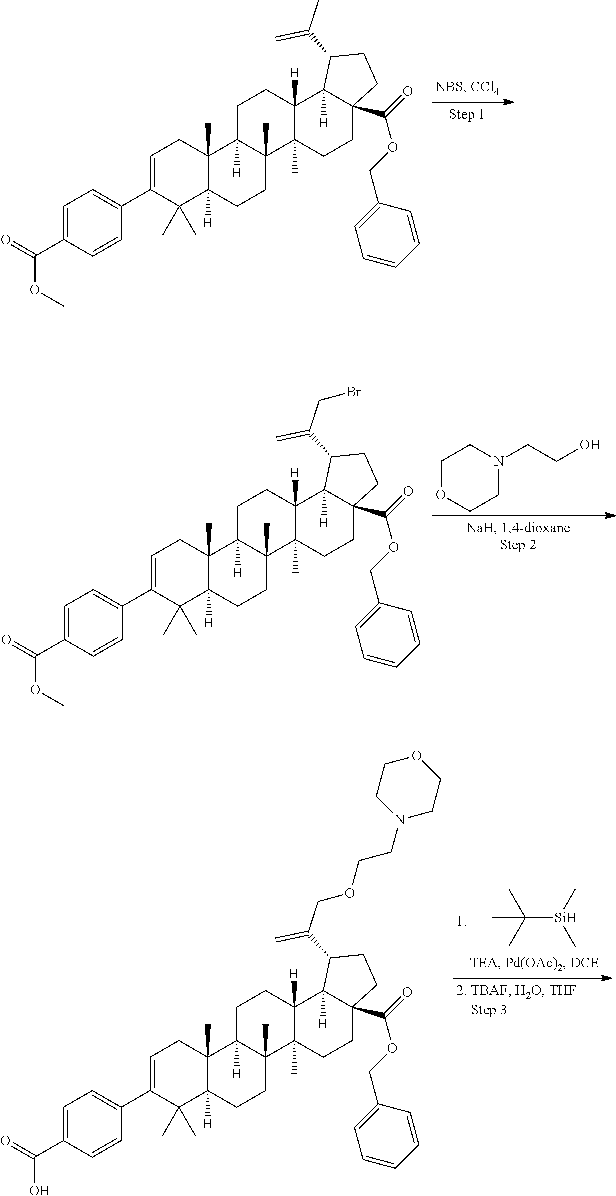

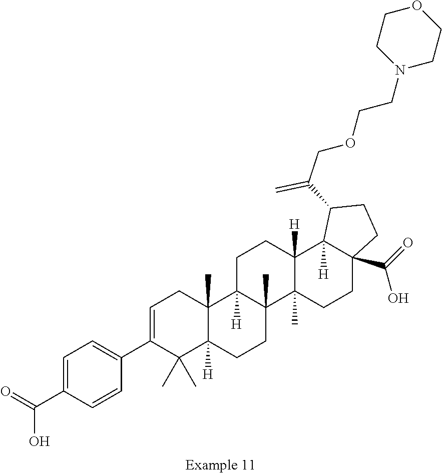

### Step 1: Preparation of (1R,3aS,5aR,5bR,7aR,11aS,11bR,13aR,13bS)-benzyl 1-(3-bromoprop-1-en-2-yl)-9-(4-(methoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylate

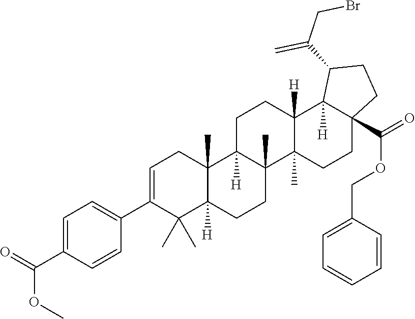

To a solution of (1R,3aS,5aR,5bR,7aR,11aS,11bR,13aR,13bR)-benzyl 9-(4-(methoxycarbonyl)phenyl)-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysene-3a-carboxylate (3.25 g, 4.90 mmol) in carbon tetrachloride (25 mL) was added N-bromosuccinimide (1.00 g, 5.62 mmol). The mixture was stirred at rt for 1 h, and an additional 0.25 g of N-bromosuccinimide was added. After stirring the mixture for 18 h at rt, it was filtered through a pad of celite (washed with DCM) and the filtrate was concentrated under reduced pressure. The residue was purified by flash chromatography using a 160 silica gel column and a 0-10% ethyl acetate in hexanes gradient. The fractions containing the expected product were combined and concentrated under reduced pressure to give 1.44 g of the expected product as a white solid. Several less pure fractions were combined, concentrated, and repurified by flash chromatography (0-5% ethyl acetate in hexanes, 90 g silica gel column). The isolates were combined to give the product (2.1 g, 2.83 mmol, 57.7% yield) as a white solid. LC/MS: m/e 741, 743.2 (M+H)⁺, 4.13 minutes (method 1). ¹H NMR (500 MHz, CHLOROFORM-d) δ ppm 7.93 (d, J=8.2Hz, 2H), 7.44-7.31 (m, 5H), 7.20 (d, J=8.2Hz, 2H), 5.29 (dd, J=6.1, 1.5 Hz, 1H), 5.23-5.08 (m, 3H), 5.05 (s, 1H), 4.03-3.97 (m, 2H), 3.92 (s, 3H), 3.10 (d, J=4.3 Hz, 1H), 2.34 (dt, J=12.6, 3.0 Hz, 1H), 2.27-2.19 (m, 1H), 2.15-2.05 (m, 2H), 1.92 (dd, J=12.7, 7.8 Hz, 1H), 1.79 (t, J=11.3 Hz, 1H), 1.00 (s, 3H), 0.96 (s, 3H), 0.93 (s, 3H), 0.92 (s, 3H), 1.75-0.90 (m, 16H), 0.82 (s, 3H).

### Step 2: Preparation of 4-((1R,3aS,5aR,5bR,7aR,11aS,11bR,13aR,13bS)-3a-(benzyloxycarbonyl)-5a,5b,8,8,11a-pentamethyl-1-(3-(2-morpholinoethoxy)prop-1-en-2-yl)-2,3,3a,4,5,5a,5b,6,7,7a,8,11,11a,11b,12,13,13a,13b-octadecahydro-1H-cyclopenta[a]chrysen-9-yl)benzoic acid

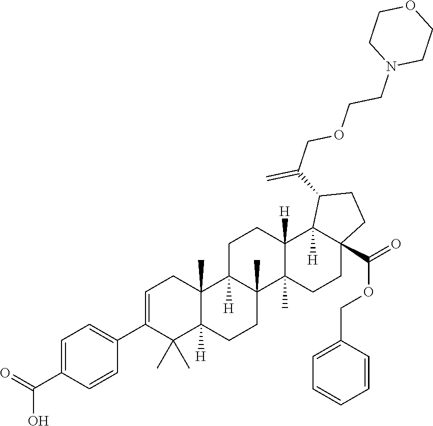

To a suspension of NaH (60% mineral oil dispersion) (0.135 g, 3.37 mmol) in 1,4-dioxane (3 mL) was added 4-(2-hydroxyethyl)morpholine (0.204 mL, 1.685 mmol) and (1R,3aS,5aR,5bR,7aR,11aS,11bR,13aR,13bS)-benzyl 1-(3-bromoprop-1-en-2-yl)-9-(4-(methoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylate (0.25 g, 0.337 mmol). The mixture was stirred at rt then was heated to 50° C. for 20 h. The reaction was cooled to rt and quenched with water (10 mL) then was extracted with dichloromethane (3×20 mL). The combined organic layers were dried with sodium sulfate, filtered, and concentrated under reduced pressure. The residue was purified by flash chromatography using a 0-10% MeOH in dichloromethane gradient and a 12 g silica gel column. The fractions containing the expected product were combined and concentrated under reduced pressure to give the title compound (0.133 g, 0.171 mmol, 50.7% yield) as a white foam. LC/MS: m/e 778.4 (M+H)⁺, 2.44 minutes (method 1).

Step 3: To a solution of 4-((1R,3aS,5aR,5bR,7aR,11aS,11bR,13aR,13bS)-3a-(benzyloxycarbonyl)-5a,5b,8,8,11a-pentamethyl-1-(3-(2-morpholinoethoxy)prop-1-en-2-yl)-2,3,3a,4,5,5a,5b,6,7,7a,8,11,11a,11b,12,13,13a,13b-octadecahydro-1H-cyclopenta[a]chrysen-9-yl)benzoic acid (0.078 g, 0.00 μmol) in DCE (2 mL) was added triethylamine (0.022 mL, 0.160 mmol), tert-butyldimethylsilane (0.033 mL, 0.200 mmol), and palladium (II) acetate (0.011 g, 0.050 mmol). The mixture was flushed with nitrogen and was heated to 60° C. for 5.5 h, then was cooled to rt and stirred overnight. The mixture was filtered through a pad of celite to remove the solids (washed with dichloromethane) and the filtrate was concentrated under reduced pressure. The residue was dissolved in THF (2 mL) and was treated with tetrabutylammonium fluoride hydrate (0.042 g, 0.150 mmol). After 1.25 h, the mixture was diluted with water (5 mL) and extracted with dichloromethane (3×7 mL). The combined organic layers were dried with sodium sulfate, filtered, and concentrated under reduced pressure. The residue was purified by crystallization from hot dioxane and water. The solids that formed upon cooling were collected by filtration and were washed with water. The solids that were collected were recrystallized from hot ethanol, dioxane, and water was added slowly. The solids that formed were collected by filtration and were washed with ethanol to give the product (10 mg, 0.0145 mmol, 14.5% yield) as an off-white solid. LC/MS: m/e 688.4 (M+H)⁺, 2.18 minutes (method 1). ¹H NMR (500 MHz, acetic acid d₄) δ ppm 8.03 (d, J=8.2Hz, 2H), 7.30 (d, J=8.2Hz, 2H), 5.37 (d, J=4.3 Hz, 1H), 5.03 (br. s., 1H), 5.02 (br. s., 1H), 4.11-3.99 (m, 6H), 3.95-3.90 (m, 2H), 3.49-3.45 (m, 2H), 3.00-2.92 (m, 1H), 2.39-2.30 (m, 2H), 1.10 (s, 3H), 1.07 (s, 6H), 2.23-1.05 (m, 24H), 1.01 (s, 3H), 1.00 (s, 3H).

### Example 12

### Preparation of 4-((1R,3aS,5aR,5bR,7aR,11aS,11bR,13aR,13bS)-3a-(2-carboxyethylcarbamoyl)-5a,5b,8,8,11a-pentamethyl-1-(3-(2-morpholinoethoxy)prop-1-en-2-yl)-2,3,3a,4,5,5a,5b,6,7,7a,8,11,11a,11b,12,13,13a,13b-octadecahydro-1H-cyclopenta[a]chrysen-9-yl)benzoic acid

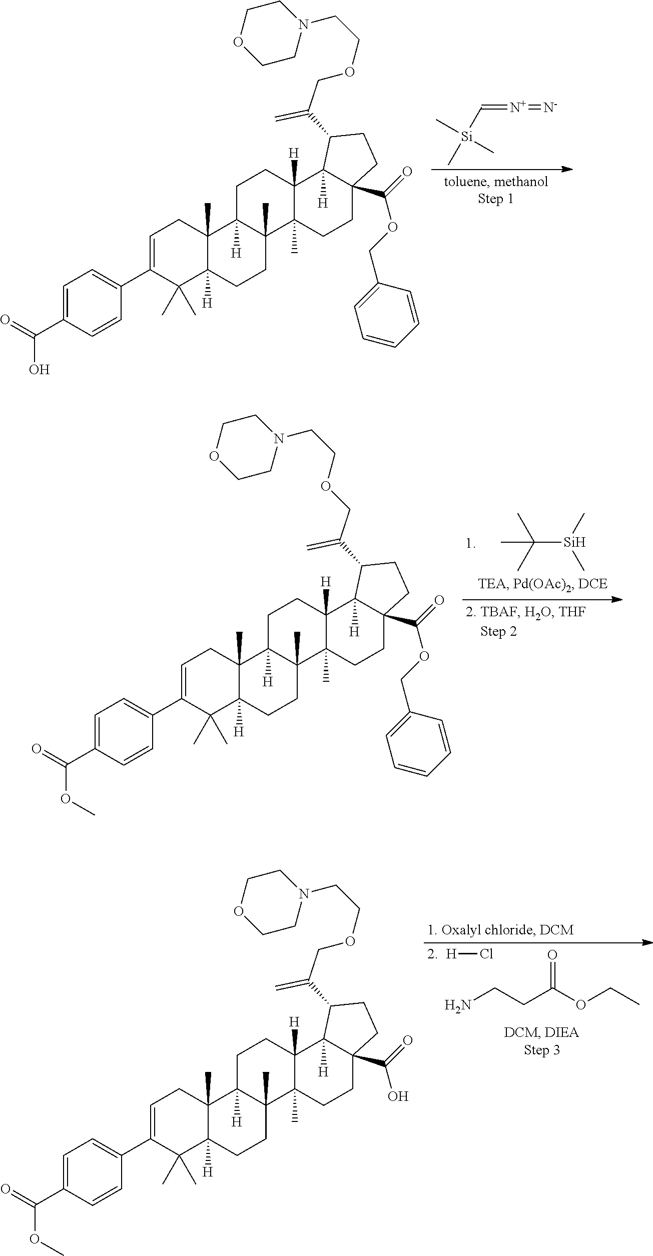

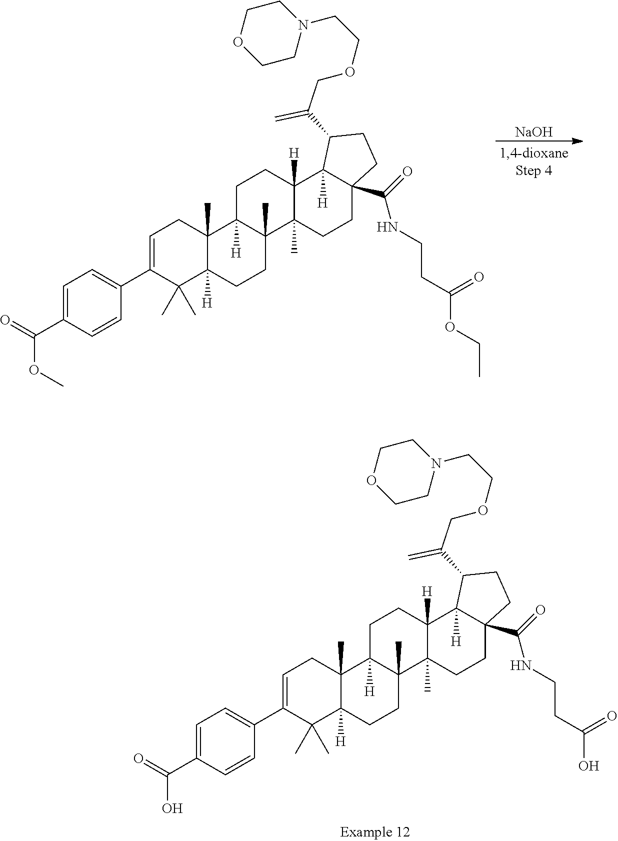

### Step 1: Preparation of (1R,3aS,5aR,5bR,7aR,11aS,11bR,13aR,13bS)-benzyl 9-(4-(methoxycarbonyl)phenyl)-5a,5b,8,8,11a-pentamethyl-1-(3-(2-morpholinoethoxy)prop-1-en-2-yl)-2,3,3a,4,5,5a,5b,6,7,7a,8,11,11a,11b,12,13,13a,13b-octadecahydro-1H-cyclopenta[a]chrysene-3a-carboxylate

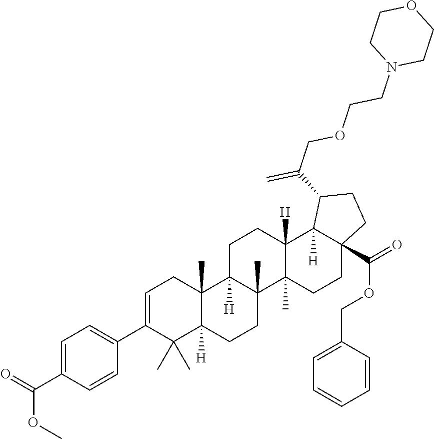

A cloudy solution of 4-((1R,3aS,5aR,5bR,7aR,11aS,11bR,13aR,13bS)-3a-(benzyloxycarbonyl)-5a,5b,8,8,11a-pentamethyl-1-(3-(2-morpholinoethoxy)prop-1-en-2-yl)-2,3,3a,4,5,5a,5b,6,7,7a,8,11,11a,11b,12,13,13a,13b-octadecahydro-1H-cyclopenta[a]chrysen-9-yl)benzoic acid (0.38 g, 0.488 mmol) in toluene (3 mL) and methanol (0.75 mL) was cooled to 0° C. and TMS-diazomethane (2M in hexanes) (0.317 mL, 0.635 mmol) was added dropwise. The solution bubbled vigorously for five minutes, then bubbling ceased. The mixture was warmed to rt and stirred. After 4 h of stirring, an additional 0.1 mL of 2N TMS-diazomethane solution was added and the mixture was stirred at rt for 1 h. The mixture was diluted with 20 mL of ethyl acetate and was washed with sat. sodium bicarbonate followed by sat. sodium chloride. The organic layer was dried with sodium sulfate, filtered, and concentrated under reduced pressure. The residue was purified by flash chromatography using a 0-5% MeOH in dichloromethane gradient and a 25 g silica gel column. The fractions containing the expected product were combined and concentrated under reduced pressure to give the product (0.295 g, 0.350 mmol, 71.7% yield) as a white foam. LC/MS: m/e 792.4 (M+H)⁺, 3.51 minutes (method 1). ¹H NMR (500 MHz, CHLOROFORM-d) δ ppm 7.92 (d, J=8.24 Hz, 2H), 7.29-7.39 (m, 5H), 7.18 (d, J=7.93 Hz, 2H), 5.27 (d, J=5.19 Hz, 1H), 5.06-5.18 (m, 2H), 4.93 (s, 1H), 4.91 (s, 1H), 3.94 (s, 2H), 3.90 (s, 3H), 3.72 (t, J=4.58 Hz, 4H), 3.57 (t, J=5.80 Hz, 2H), 2.87-2.95 (m, 1H), 2.60 (t, J=5.80 Hz, 2H),2.52 (br. s., 4H),2.27-2.33 (m, 1H),2.20 (td, J=12.28, 3.20 Hz, 1H),2.08 (dd, J=17.09, 6.10 Hz, 1H), 1.85-2.00 (m, 2H),1.73 (t, J=11.29 Hz, 1H),1.64 (d, J=16.79 Hz, 1H), 0.98-1.53 (m, 15H),0.97 (s, 3H),0.94 (s, 3H),0.91 (s, 3H),0.90 (s, 3H),0.80 (s, 3H).

### Step 2: Preparation of (1R,3aS,5aR,5bR,7aR,11aS,11bR,13aR,13bS)-9-(4-(methoxycarbonyl)phenyl)-5a,5b,8,8,11a-pentamethyl-1-(3-(2-morpholinoethoxy)prop-1-en-2-yl)-2,3,3a,4,5,5a,5b,6,7,7a,8,11,11a,11b,12,13,13a,13b-octadecahydro-1H-cyclopenta[a]chrysene-3a-carboxylic acid

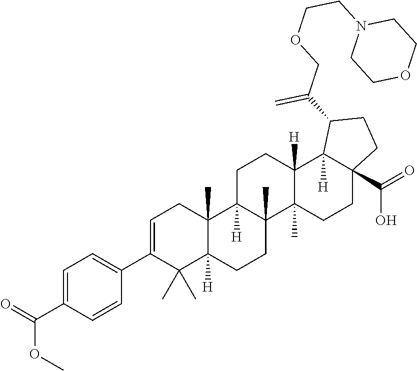

To a solution of (1R,3aS,5aR,5bR,7aR,11aS,11bR,13aR,13bS)-benzyl 9-(4-(methoxycarbonyl)phenyl)-5a,5b,8,8,11a-pentamethyl-1-(3-(2-morpholino ethoxy)prop-1-en-2-yl)-2,3,3a,4,5,5a,5b,6,7,7a,8,11,11a,11b,12,13,13a,13b-octadecahydro-1H-cyclopenta[a]chrysene-3a-carboxylate (0.288 g, 0.364 mmol) in DCE (3.5 mL) was added TEA (0.081 mL, 0.582 mmol), tert-butyldimethylsilane (0.121 mL, 0.727 mmol), and palladium acetate (0.020 g, 0.091 mmol). The mixture was flushed with N2, and was heated to 60° C. After 2.5 h the mixture was cooled to rt and filtered through a pad of celite to remove the solids then was concentrated under reduced pressure. The residue was diluted with 5 mL of THF and to the cloudy solution was added tetrabutylammonium fluoride hydrate (0.152 g, 0.545 mmol). The mixture was stirred at rt for 2 h, diluted with water (15 mL), and extracted with ethyl acetate (3×15 mL). The combined organic layers were dried over sodium sulfate, filtered, and concentrated under reduced pressure. The residue was purified by flash chromatography using a 0-5% methanol in dichloromethane gradient and a 25 g silica gel column. The fractions containing the expected product were combined and concentrated under reduced pressure to give the product (0.188 g, 0.268 mmol, 73.7% yield) as a white solid. LC/MS: m/e 702.4 (M+H)⁺, 2.66 minutes (method 1). ¹H NMR (500 MHz, CHLOROFORM-d) δ ppm 7.90 (d, J=8.24 Hz, 2H) 7.16 (d, J=8.24 Hz, 2H), 5.26 (d, J=4.88 Hz, 1H), 4.92 (d, J=1.83 Hz, 2H), 3.93 (s, 2H), 3.88 (s, 3H), 3.74 (t, J=4.43 Hz, 4H), 3.62 (t, J=5.49 Hz, 2H), 2.89 (td, J=10.83, 4.27 Hz, 1H), 2.58-2.74 (m, 6H), 2.21-2.32 (m, 2H), 1.99-2.13 (m, 2H), 1.91 (dd, J=12.05, 8.09 Hz, 1H), 1.72 (t, J=11.29 Hz, 1H), 1.65 (d, J=16.79 Hz, 1H), 1.01-1.56 (m, 15H), 0.99 (s, 3H), 0.98 (s, 3H), 0.95 (s, 3H), 0.90 (s, 6H).

### Step 3: Preparation of methyl 4-((1R,3aS,5aR,5bR,7aR,11aS,11bR,13aR,13bS)-3a-(3-ethoxy-3-oxopropylcarbamoyl)-5a,5b,8,8,11a-pentamethyl-1-(3-(2-morpholinoethoxy)prop-1-en-2-yl)-2,3,3a,4,5,5a,5b,6,7,7a,8,11,11a,11b,12,13,13a,13b-octadecahydro-1H-cyclopenta[a]chrysen-9-yl)benzoate

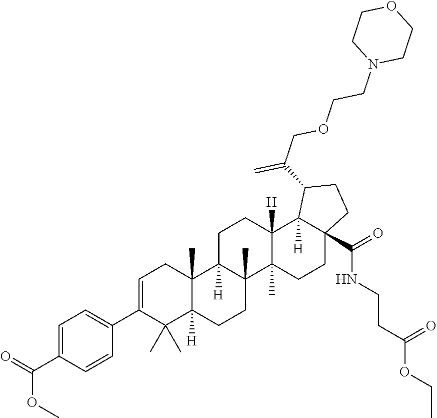

To a flask containing (1R,3aS,5aR,5bR,7aR,11aS,11bR,13aR,13bS)-9-(4-(methoxycarbonyl)phenyl)-5a,5b,8,8,11a-pentamethyl-1-(3-(2-morpholinoethoxy)prop-1-en-2-yl)-2,3,3a,4,5,5a,5b,6,7,7a,8,11,11a,11b,12,13,13a,13b-octadecahydro-1H-cyclopenta[a]chrysene-3a-carboxylic acid (0.188 g, 0.268 mmol) was added oxalyl chloride (2M in DCM) (3 mL, 6.00 mmol). The solution (which bubbled for several minutes upon addition of oxalyl chloride) was stirred at rt for 2 h, then was concentrated under reduced pressure. The residue was dissolved in DCM and was concentrated two additional times to remove any remaining oxalyl chloride. The crude acid chloride product was used in the next step with no additional purification. To a suspension of methyl 4-((1R,3aS,5aR,5bR,7aR,11aS,11bR,13aR,13bS)-3a-(chlorocarbonyl)-5a,5b,8,8,11a-pentamethyl-1-(3-(2-morpholinoethoxy)prop-1-en-2-yl)-2,3,3a,4,5,5a,5b,6,7,7a,8,11,11a,11b,12,13,13a,13b-octadecahydro-1H-cyclopenta[a]chrysen-9-yl)benzoate (32.2 mg, 0.0447 mmol) in DCE (1 mL) was added beta-alanine, ethyl ester hydrochloride (10.30 mg, 0.067 mmol) and diisopropylethylamine (0.023 mL, 0.134 mmol). The mixture was stirred at rt for 5 h then was purified directly by flash chromatography using a 0-5% MeOH in DCM gradient with 0.1% ammonium acetate added to the mixture. The fractions containing the expected product were combined and concentrated under reduced pressure to give the product (32 mg, 0.040 mmol, 89% yield) as a white foam. LC/MS: m/e 801.4 (M+H)⁺, 2.66 minutes (method 1).

Step 4: To a solution of methyl 4-((1R,3aS,5aR,5bR,7aR,11aS,11bR,13aR,13bS)-3a-(3-ethoxy-3-oxopropylcarbamoyl)-5a,5b,8,8,11a-pentamethyl-1-(3-(2-morpholinoethoxy)prop-1-en-2-yl)-2,3,3a,4,5,5a,5b,6,7,7a,8,11,11a,11b,12,13,13a,13b-octadecahydro-1H-cyclopenta[a]chrysen-9-yl)benzoate (0.032 g, 0.040 mmol) in 1,4-dioxane (1 mL) was added NaOH (1N) (0.199 mL, 0.199 mmol). The mixture was heated to 75° C. for 15 h then was cooled to rt and purified by prep HPLC (method 1). The fractions containing the expected product were combined and concentrated under reduced pressure to give the product (18 mg, 0.024 mmol, 59.6% yield) as a white solid. LC/MS: m/e 759.4 (M+H)⁺, 1.99 minutes (method 1). ¹H NMR (500 MHz, CHLOROFORM-d) δ ppm 7.94 (d, J=8.24 Hz, 2H), 7.16 (d, J=8.24 Hz, 2H), 6.83 (t, J=5.49 Hz, 1H), 5.17 (d, J=4.58 Hz, 1H), 4.90 (s, 1H), 4.93 (s, 1H), 3.91-4.00 (m, 2H), 3.90 (t, J=4.58 Hz, 4H), 3.78-3.85 (m, 1H), 3.56-3.72 (m, 3H), 3.00-3.13 (m, 6H), 2.75-2.82 (m, 1H), 2.49-2.63 (m, 2H), 2.38-2.44 (m, 1H), 2.10-2.20 (m, 2H), 1.81-2.00 (m, 3H), 1.03 (s, 3H), 1.01-1.67 (m, 16H), 0.99 (s, 3H), 0.91 (s, 3H), 0.89 (s, 6H).

### Examples 13-17

### General Scheme for the Preparation of C-28 amides with the C-30 ethyl Morpholino Ether

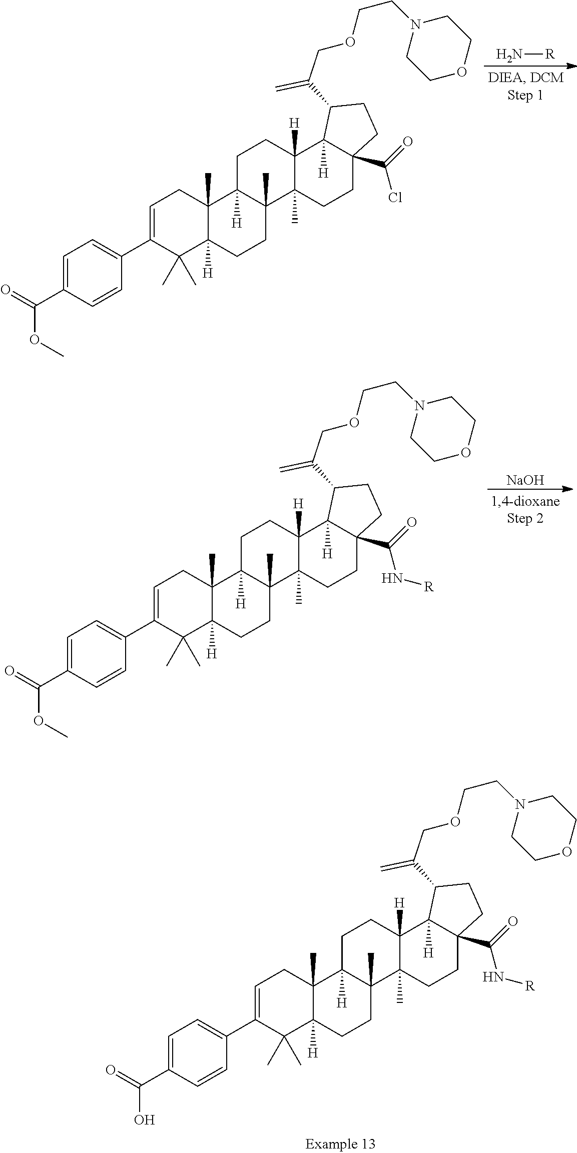

### Example 13

### Preparation of 4-((1R,3aS,5aR,5bR,7aR,11aS,11bR,13aR,13bS)-3a-(2-(2-hydroxyethylamino)ethylcarbamoyl)-5a,5b,8,8,11a-pentamethyl-1-(3-(2-morpholinoethoxy)prop-1-en-2-yl)-2,3,3a,4,5,5a,5b,6,7,7a,8,11,11a,11b,12,13,13a,13b-octadecahydro-1H-cyclopenta[a]chrysen-9-yl)benzoic acid

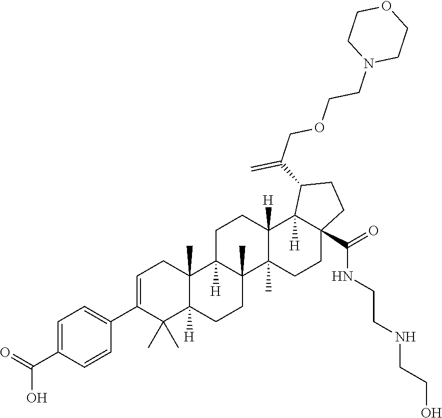

### Step 1: Preparation of methyl 4-((1R,3aS,5aR,5bR,7aR,11aS,11bR,13aR,13bS)-3a-(2-(2-hydroxyethylamino)ethylcarbamoyl)-5a,5b,8,8,11a-pentamethyl-1-(3-(2-morpholinoethoxy)prop-1-en-2-yl)-2,3,3a,4,5,5a,5b,6,7,7a,8,11,11a,11b,12,13,13a,13b-octadecahydro-1H-cyclopenta[a]chrysen-9-yl)benzoate

To a suspension of methyl 4-((1R,3aS,5aR,5bR,7aR,11aS,11bR,13aR,13bS)-3a-(chlorocarbonyl)-5a,5b,8,8,11a-pentamethyl-1-(3-(2-morpholinoethoxy)prop-1-en-2-yl)-2,3,3a,4,5,5a,5b,6,7,7a,8,11,11a,11b,12,13,13a,13b-octadecahydro-1H-cyclopenta[a]chrysen-9-yl)benzoate (32.2 mg, 0.0447 mmol) (synthesis described above in the preparation of methyl 4-((1R,3aS,5aR,5bR,7aR,11aS,11bR,13aR,13bS)-3a-(3-ethoxy-3-oxopropylcarbamoyl)-5a,5b,8,8,11a-pentamethyl-1-(3-(2-morpholinoethoxy)prop-1-en-2-yl)-2,3,3a,4,5,5a,5b,6,7,7a,8,11,11a,11b,12,13,13a,13b-octadecahydro-1H-cyclopenta[a]chrysen-9-yl)benzoate) in DCE (1 mL) was added 2-(2-Aminoethylamino)ethanol (16 μL, 0.158 mmol). The mixture was stirred at rt overnight. After 16 h of stirring, to the mixture was added diisopropylethylamine (0.023 mL, 0.134 mmol). The mixture was stirred for 1 h at rt then was directly purified by flash chromatography using a 0-10% methanol in dichloromethane gradient with 0.1% ammonium hydroxide added to the mixture. The fractions containing the expected product were combined and concentrated under reduced pressure to give the product (19 mg, 0.024 mmol, 53.9% yield) as a white foam. LC/MS: m/e 788.4 (M+H)⁺, 2.52 minutes (method 1). ¹H NMR (500 MHz, CHLOROFORM-d) δ ppm 7.91 (d, J=8.24 Hz, 2H), 7.18 (d, J=8.24 Hz, 2H), 6.57 (br. s., 1H), 5.27 (d, J=4.58 Hz, 1H), 4.92 (s, 1H), 4.91 (s, 1H), 3.94 (s, 2H), 3.89 (s, 3H), 3.74-3.80 (m, 2H), 3.70-3.74 (m, 4H), 3.39-3.63 (m, 5H), 2.88-3.06 (m, 6H), 2.59 (t, J=5.80 Hz, 2H), 2.41-2.56 (m, 5H), 1.93-2.13 (m, 3H), 1.80 (dd, J=12.21, 7.63 Hz, 1H), 0.98-1.74 (m, 17H), 0.99 (s, 3H), 0.97 (s, 3H), 0.95 (s, 3H), 0.87-0.93 (m, 6H).

Step 2: To a solution of methyl 4-((1R,3aS,5aR,5bR,7aR,11aS,11bR,13aR,13bS)-3a-(2-(2-hydroxyethylamino)ethylcarbamoyl)-5a,5b,8,8,11a-pentamethyl-1-(3-(2-morpholinoethoxy)prop-1-en-2-yl)-2,3,3a,4,5,5a,5b,6,7,7a,8,11,11a,11b,12,13,13a,13b-octadecahydro-1H-cyclopenta[a]chrysen-9-yl)benzoate (0.019 g, 0.024 mmol) in 1,4-dioxane (1 mL) was added 1N NaOH (0.121 mL, 0.121 mmol). The mixture was heated to 75° C. for 23 h then was cooled to rt and stirred for an additional 63 h. To the reaction was added an additional 0.1 mL of 1N NaOH and the mixture was heated to 75° C. for 23 h then was cooled to rt and was purified by prep HPLC (method 1). The fractions containing the expected product were combined and concentrated under reduced pressure to give the product (15 mg, 0.019 mmol, 80% yield) as a white solid. LC/MS: m/e 774.6 (M+H)⁺, 2.07 minutes (method 1). ¹H NMR (500 MHz, Acetic) δ ppm 8.03 (d, J=8.2Hz, 2H), 7.29 (d, J=8.5Hz, 2H), 5.37 (d, J=4.6 Hz, 1H), 5.01 (br. s., 1H), 5.00 (br. s., 1H), 4.09-3.96 (m, 8H), 3.91 (br. s., 2H), 3.78-3.71 (m, 1H), 3.70-3.63 (m, 1H), 3.54-3.44 (m, 3H), 3.38-3.32 (m, 4H), 3.04 (td, J=10.6, 3.5Hz, 1H), 2.56-2.49 (m, 1H), 1.09 (s, 3H), 1.06 (s, 3H), 1.05 (s, 3H), 1.01 (s, 3H), 0.99 (s, 3H), 2.24-0.97 (m, 24H).

### Example 14

### Preparation of 4-((1R,3aS,5aR,5bR,7aR,11aS,11bR,13aR,13bS)-3a-(2-(dimethylamino)ethylcarbamoyl)-5a,5b,8,8,11a-pentamethyl-1-(3-(2-morpholinoethoxy)prop-1-en-2-yl)-2,3,3a,4,5,5a,5b,6,7,7a,8,11,11a,11b,12,13,13a,13b-octadecahydro-1H-cyclopenta[a]chrysen-9-yl)benzoic acid

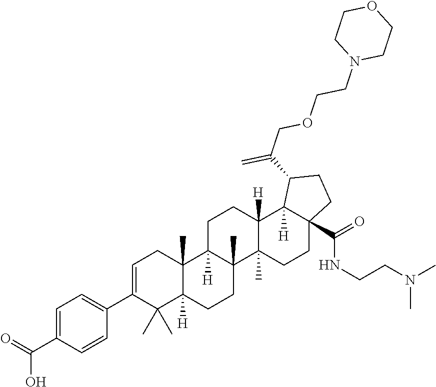

### Step 1: Preparation of methyl 4-((1R,3aS,5aR,5bR,7aR,11aS,11bR,13aR,13bS)-3a-(2-(dimethylamino)ethylcarbamoyl)-5a,5b,8,8,11a-pentamethyl-1-(3-(2-morpholinoethoxy)prop-1-en-2-yl)-2,3,3a,4,5,5a,5b,6,7,7a,8,11,11a,11b,12,13,13a,13b-octadecahydro-1H-cyclopenta[a]chrysen-9-yl)benzoate

To a suspension of methyl 4-((1R,3aS,5aR,5bR,7aR,11aS,11bR,13aR,13bS)-3a-(chlorocarbonyl)-5a,5b,8,8,11a-pentamethyl-1-(3-(2-morpholinoethoxy)prop-1-en-2-yl)-2,3,3a,4,5,5a,5b,6,7,7a,8,11,11a,11b,12,13,13a,13b-octadecahydro-1H-cyclopenta[a]chrysen-9-yl)benzoate (40 mg, 0.056 mmol) (synthesis described above in the preparation of methyl 4-((1R,3aS,5aR,5bR,7aR,11aS,11bR,13aR,13bS)-3a-(3-ethoxy-3-oxopropylcarbamoyl)-5a,5b,8,8,11a-pentamethyl-1-(3-(2-morpholinoethoxy)prop-1-en-2-yl)-2,3,3a,4,5,5a,5b,6,7,7a,8,11,11a,11b,12,13,13a,13b-octadecahydro-1H-cyclopenta[a]chrysen-9-yl)benzoate) in DCE (1 mL) was added N,N-dimethylaminoethylamine (7.34 mg, 0.083 mmol). The mixture was stirred at rt for 16 h then to the mixture was added diisopropylethylamine (0.023 mL, 0.134 mmol). It was stirred for an additional 2 h at rt then was directly purified by flash chromatography using a 0-10% MeOH in DCM gradient with 0.1% ammonium hydroxide added to the mixture and a 12 g silica gel column. The fractions containing the product were combined and concentrated under reduced pressure to give expected product (38.4 mg, 0.050 mmol, 90% yield) as a white foam. LC/MS: m/e 772.5 (M+H)⁺, 2.68 minutes (method 1).

Step 2: To a solution of methyl 4-((1R,3aS,5aR,5bR,7aR,11aS,11bR,13aR,13bS)-3a-(2-(dimethylamino)ethylcarbamoyl)-5a,5b,8,8,11a-pentamethyl-1-(3-(2-morpholinoethoxy)prop-1-en-2-yl)-2,3,3a,4,5,5a,5b,6,7,7a,8,11,11a,11b,12,13,13a,13b-octadecahydro-1H-cyclopenta[a]chrysen-9-yl)benzoate (38.4 mg, 0.050 mmol) in 1,4-dioxane (1 mL) was added 1N NaOH (0.249 mL, 0.249 mmol). The mixture was heated to 75° C. for 15 h then was purified by prep HPLC (method 1). The fractions containing the product were combined and concentrated under reduced pressure to give the product (14 mg, 0.018 mmol, 37% yield) as a white solid. LC/MS: m/e 758.6 (M+H)⁺, 2.14 minutes (method 1). ¹H NMR (500 MHz, CHLOROFORM-d) δ ppm 7.93 (d, J=7.93 Hz, 2H), 7.15 (d, J=7.94 Hz, 2H), 7.11 (br. s., 1H), 5.28 (d, J=4.88 Hz, 1H), 4.91 (s, 1H), 4.89 (s, 1H), 3.88-3.96 (m, 2H), 3.74 (t, J=4.58 Hz, 4H), 3.59 (t, J=5.49 Hz, 2H), 3.47-3.53 (m, 2H), 2.96-3.04 (m, 1H), 2.74-2.82 (m, 2H), 2.61-2.68 (m, 2H), 2.58 (br. s., 4H), 2.51 (s, 6H), 2.39-2.47 (m, 1H), 1.93-2.11 (m, 3H), 1.75-1.83 (m, 1H), 1.58-1.69 (m, 2H), 0.96-1.53 (m, 15H), 0.96 (s, 3H), 0.94 (s, 6H), 0.91 (s, 6H).

### Example 15

### Preparation of 4-((1R,3aS,5aR,5bR,7aR,11aS,11bR,13aR,13bS)-5a,5b,8,8,11a-pentamethyl-1-(3-(2-morpholinoethoxy)prop-1-en-2-yl)-3a-(3-(2-oxopyrrolidin-1-yl)propylcarbamoyl)-2,3,3a,4,5,5a,5b,6,7,7a,8,11,11a,11b,12,13,13a,13b-octadecahydro-1H-cyclopenta[a]chrysen-9-yl)benzoic acid

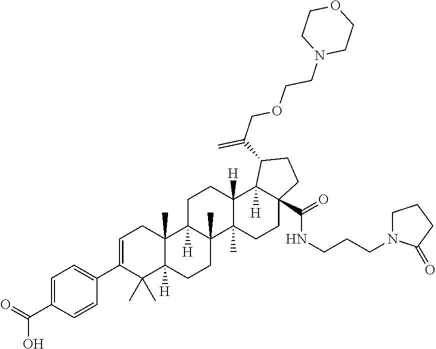

### Step 1: Preparation of methyl 4-((1R,3aS,5aR,5bR,7aR,11aS,11bR,13aR,13bS)-5a,5b,8,8,11a-pentamethyl-1-(3-(2-morpholinoethoxy)prop-1-en-2-yl)-3a-(3-(2-oxopyrrolidin-1-yl)propylcarbamoyl)-2,3,3a,4,5,5a,5b,6,7,7a,8,11,11a,11b,12,13,13a,13b-octadecahydro-1H-cyclopenta[a]chrysen-9-yl)benzoate

To a suspension of methyl 4-((1R,3aS,5aR,5bR,7aR,11aS,11bR,13aR,13bS)-3a-(chlorocarbonyl)-5a,5b,8,8,11a-pentamethyl-1-(3-(2-morpholinoethoxy)prop-1-en-2-yl)-2,3,3a,4,5,5a,5b,6,7,7a,8,11,11a,11b,12,13,13a,13b-octadecahydro-1H-cyclopenta[a]chrysen-9-yl)benzoate (32.2 mg, 0.0447 mmol) (synthesis described above in the preparation of methyl 4-((1R,3aS,5aR,5bR,7aR,11aS,11bR,13aR,13bS)-3a-(3-ethoxy-3-oxopropylcarbamoyl)-5a,5b,8,8,11a-pentamethyl-1-(3-(2-morpholinoethoxy)prop-1-en-2-yl)-2,3,3a,4,5,5a,5b,6,7,7a,8,11,11a,11b,12,13,13a,13b-octadecahydro-1H-cyclopenta[a]chrysen-9-yl)benzoate) in DCE (1 mL) was added 1-(3-Aminopropyl)-2-pyrrolidinone (9.40 μL, 0.067 mmol). The mixture was stirred at rt for 16 h then diisopropylethylamine (0.023 mL, 0.134 mmol) was added to the mixture and it was stirred for an additional 1 h at rt. The mixture was purified by flash chromatography using a 0-5% methanol in dichloromethane gradient with 0.1% ammonium acetate added to the mixture and a 12 g silica gel column. The fractions containing the product were combined and concentrated under reduced pressure to give the product (36.4 mg, 0.044 mmol, 99% yield) as a white foam. LC/MS: m/e 826.5 (M+H)⁺, 2.59 minutes (method 1). ¹H NMR (500 MHz, CHLOROFORM-d) δ ppm 7.92 (d, J=8.24 Hz, 2H), 7.18 (d, J=7.93 Hz, 2H), 6.91 (t, J=6.10 Hz, 1H), 5.28 (d, J=4.88 Hz, 1H), 4.92 (br. s., 2H), 3.95 (br. s., 2H), 3.90 (s, 3H), 3.72 (t, J=4.58 Hz, 4H), 3.58 (t, J=5.04 Hz, 2H), 3.33-3.48 (m, 3H), 3.20-3.33 (m, 2H),2.96-3.08 (m, 2H), 2.60 (t, J=5.80 Hz, 2H), 2.52 (br. s., 5H), 2.43 (t, J=8.09 Hz, 2H), 1.95-2.18 (m, 5H), 1.81 (dd, J=11.90, 7.93 Hz, 1H), 0.99 (s, 3H), 0.98-1.72 (m, 19H), 0.97 (s, 3H),0.95 (s, 3H), 0.91 (s, 6H).

Step 2: To a solution of methyl 4-((1R,3aS,5aR,5bR,7aR,11aS,11bR,13aR,13bS)-5a,5b,8,8,11a-pentamethyl-1-(3-(2-morpholinoethoxy)prop-1-en-2-yl)-3a-(3-(2-oxopyrrolidin-1-yl)propylcarbamoyl)-2,3,3a,4,5,5a,5b,6,7,7a,8,11,11a,11b,12,13,13a,13b-octadecahydro-1H-cyclopenta[a]chrysen-9-yl)benzoate (0.0364 g, 0.044 mmol) in 1,4-Dioxane (1 mL) was added 1N NaOH (0.220 mL, 0.220 mmol). The mixture was heated to 75° C. for 15 h then was purified by prep HPLC (method 1). The fractions containing the expected product were combined and concentrated under reduced pressure to give the product (31 mg, 0.035 mmol, 81% yield) as a white solid. LC/MS: m/e 812.5 (M+H)⁺, 2.11 minutes (method 1). ¹H NMR (500 MHz, CHLOROFORM-d) δ ppm 8.65 (br. s., 1H), 7.94 (d, J=7.93 Hz, 2H), 7.17 (d, J=8.24 Hz, 2H), 6.91 (t, J=6.26 Hz, 1H), 5.25 (d, J=4.88 Hz, 1H), 4.88-4.93 (m, 2H), 3.88-3.98 (m, 2H), 3.80 (t, J=4.58 Hz, 4H), 3.67 (t, J=5.49 Hz, 2H), 3.34-3.46 (m, 3H), 3.21-3.33 (m, 2H), 3.00-3.09 (m, 2H), 2.40-2.51 (m, 3H), 2.14 (d, J=12.21 Hz, 1H), 1.96-2.10 (m, 5H), 1.80 (dd, J=11.90, 7.63 Hz, 1H), 0.96-1.71 (m, 24H), 0.95 (s, 3H), 0.95 (s, 3H), 0.92 (s, 3H), 0.90 (br. s., 6H).

### Example 16

### Preparation of 4-((1R,3aS,5aR,5bR,7aR,11aS,11bR,13aR,13bS)-3a-(3-(1H-imidazol-1-yl)propylcarbamoyl)-5a,5b,8,8,11a-pentamethyl-1-(3-(2-morpholinoethoxy)prop-1-en-2-yl)-2,3,3a,4,5,5a,5b,6,7,7a,8,11,11a,11b,12,13,13a,13b-octadecahydro-1H-cyclopenta[a]chrysen-9-yl)benzoic acid

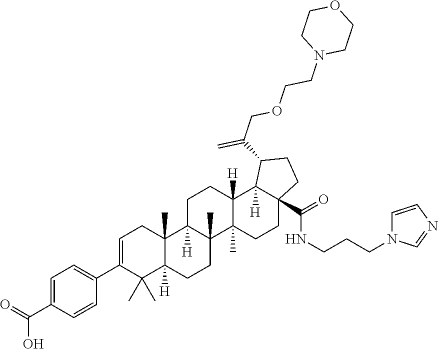

### Step 1: Preparation of methyl 4-((1R,3aS,5aR,5bR,7aR,11aS,11bR,13aR,13bS)-3a-(3-(1H-imidazol-1-yl)propylcarbamoyl)-5a,5b,8,8,11a-pentamethyl-1-(3-(2-morpholinoethoxy)prop-1-en-2-yl)-2,3,3a,4,5,5a,5b,6,7,7a,8,11,11a,11b,12,13,13a,13b-octadecahydro-1H-cyclopenta[a]chrysen-9-yl)benzoate

To a suspension of methyl 4-((1R,3aS,5aR,5bR,7aR,11aS,11bR,13aR,13bS)-3a-(chlorocarbonyl)-5a,5b,8,8,11a-pentamethyl-1-(3-(2-morpholinoethoxy)prop-1-en-2-yl)-2,3,3a,4,5,5a,5b,6,7,7a,8,11,11a,11b,12,13,13a,13b-octadecahydro-1H-cyclopenta[a]chrysen-9-yl)benzoate (25 mg, 0.035 mmol) (synthesis described above in the preparation of methyl 4-((1R,3aS,5aR,5bR,7aR,11aS,11bR,13aR,13bS)-3a-(3-ethoxy-3-oxopropylcarbamoyl)-5a,5b,8,8,11a-pentamethyl-1-(3-(2-morpholinoethoxy)prop-1-en-2-yl)-2,3,3a,4,5,5a,5b,6,7,7a,8,11,11a,11b,12,13,13a,13b-octadecahydro-1H-cyclopenta[a]chrysen-9-yl)benzoate) in DCE (1 mL) was added 1-(3-Aminopropyl)imidazole (8 μL, 0.067 mmol). The mixture was stirred at rt for 16 h then diisopropylethylamine (0.023 mL, 0.132 mmol) was added. The mixture was stirred for 30 minutes at rt then was directly purified by flash chromatography using a 0-5% methanol in dichloromethane gradient with 0.1% ammonium hydroxide added and a 12 g silica gel column. The fractions containing the expected product were combined and concentrated under reduced pressure to give the product (10.8 mg, 0.013 mmol, 38.5% yield) as a white foam. LC/MS: m/e 809.4 (M+H)⁺, 2.55 minutes (method 1). ¹H NMR (500 MHz, CHLOROFORM-d) δ ppm 7.91 (d, J=7.93 Hz, 2H), 7.49 (s, 1H), 7.18 (d, J=7.93 Hz, 2H), 7.07 (s, 1H), 6.94 (s, 1H), 5.69 (t, J=5.95Hz, 1H), 5.27 (d, J=4.88 Hz, 1H), 4.93 (s, 1H), 4.91 (s, 1H), 3.99 (td, J=6.94, 2.90 Hz, 2H), 3.94 (s, 2H), 3.90 (s, 3 H), 3.72 (t, J=4.73 Hz, 4H), 3.53-3.61 (m, 2H), 3.30-3.38 (m, 1H), 3.13-3.22 (m, 1H), 3.00 (td, J=11.14, 3.97 Hz, 1H), 2.60 (t, J=5.80 Hz, 2H), 2.42-2.55 (m, 5H), 2.09 (dd, J=17.24, 6.26 Hz, 1H), 1.94-2.04 (m, 3H), 1.88 (d, J=13.73 Hz, 1H), 0.99-1.75 (m, 18H), 0.99 (s, 3H), 0.98 (s, 3H), 0.95 (s, 3H), 0.91 (s, 6H).

Step 2: To a solution of methyl 4-((1R,3aS,5aR,5bR,7aR,11aS,11bR,13aR,13bS)-3a-(3-(1H-imidazol-1-yl)propylcarbamoyl)-5a,5b,8,8,11a-pentamethyl-1-(3-(2-morpholinoethoxy)prop-1-en-2-yl)-2,3,3a,4,5,5a,5b,6,7,7a,8,11,11a,11b,12,13,13a,13b-octadecahydro-1H-cyclopenta[a]chrysen-9-yl)benzoate (0.0108 g, 0.013 mmol) in 1,4-dioxane (1 mL) was added 1N NaOH (0.067 mL, 0.067 mmol). The mixture was heated to 75° C. for 15 h, then mixture was cooled to rt. An additional 0.067 μL of 1N NaOH was added to the mixture and it was heated to 75° C. After 8 h of heating, the mixture was cooled to rt and stirred for an additional 63 h at rt. The mixture was purified by prep HPLC. The fractions containing the expected product were combined and concentrated under reduced pressure to give the product (9 mg, 10.19 mmol, 76% yield) as a white solid. LC/MS: m/e 795.5 (M+H)⁺, 2.09 minutes (method 1). ¹H NMR (500 MHz, CHLOROFORM-d) δ ppm 7.95 (d, J=7.93 Hz, 2H), 7.64 (s, 1H), 7.16 (d, J=8.24 Hz, 2H), 7.12 (s, 1H), 6.95 (s, 1H), 5.71 (br. s., 1H), 5.25 (d, J=4.88 Hz, 1H), 4.93 (s, 1H), 4.91 (s, 1H), 3.96-4.06 (m, 2H), 3.94 (s, 2H), 3.73-3.81 (m, 4H), 3.61-3.67 (m, 2H), 3.34-3.46 (m, 1H), 3.11-3.20 (m, 1H), 2.97-3.05 (m, 1H), 2.59-2.76 (m, 6H), 2.45 (br. s., 1H), 1.95-2.10 (m, 4H), 1.89 (d, J=13.43 Hz, 1H), 0.96-1.74 (m, 18H), 0.97 (s, 6H), 0.94 (s, 3H), 0.89 (s, 3H), 0.86 (s, 3H).

### Example 17

### Preparation of 4-((1R,3aS,5aR,5bR,7aR,11aS,11bR,13aR,13bS)-3a-(2-(carboxymethylamino)ethylcarbamoyl)-5a,5b,8,8,11a-pentamethyl-1-(3-(2-morpholinoethoxy)prop-1-en-2-yl)-2,3,3a,4,5,5a,5b,6,7,7a,8,11,11a,11b,12,13,13a,13b-octadecahydro-1H-cyclopenta[a]chrysen-9-yl)benzoic acid

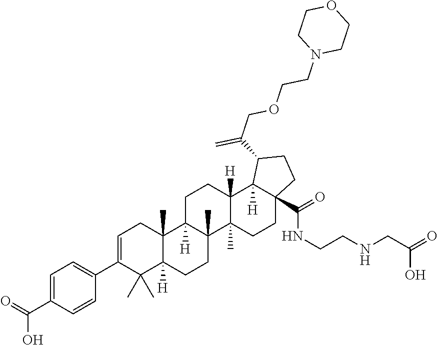

### Step 1: Preparation of methyl 4-((1R,3aS,5aR,5bR,7aR,11aS,11bR,13aR,13bS)-3a-(2-(2-methoxy-2-oxoethylamino)ethylcarbamoyl)-5a,5b,8,8,11a-pentamethyl-1-(3-(2-morpholinoethoxy)prop-1-en-2-yl)-2,3,3a,4,5,5a,5b,6,7,7a,8,11,11a,11b,12,13,13a,13b-octadecahydro-1H-cyclopenta[a]chrysen-9-yl)benzoate

To a suspension of methyl 4-((1R,3aS,5aR,5bR,7aR,11aS,11bR,13aR,13bS)-3a-(chlorocarbonyl)-5a,5b,8,8,11a-pentamethyl-1-(3-(2-morpholinoethoxy)prop-1-en-2-yl)-2,3,3a,4,5,5a,5b,6,7,7a,8,11,11a,11b,12,13,13a,13b-octadecahydro-1H-cyclopenta[a]chrysen-9-yl)benzoate (32.2 mg, 0.0447 mmol) (synthesis described above in the preparation of methyl 4-((1R,3aS,5aR,5bR,7aR,11aS,11bR,13aR,13bS)-3a-(3-ethoxy-3-oxopropylcarbamoyl)-5a,5b,8,8,11a-pentamethyl-1-(3-(2-morpholinoethoxy)prop-1-en-2-yl)-2,3,3a,4,5,5a,5b,6,7,7a,8,11,11a,11b,12,13,13a,13b-octadecahydro-1H-cyclopenta[a]chrysen-9-yl)benzoate) in DCE (1 mL) was added methyl 2-(2-aminoethylamino)acetate (8.86 mg, 0.067 mmol). The mixture was stirred at rt for 16 h at rt. To the mixture was added diisopropylethylamine (0.023 mL, 0.134 mmol) and the mixture was stirred for 3.5 h at rt. An additional 10 mg of methyl 2-(2-aminoethylamino)acetate was added and the reaction was stirred an additional 19 h at rt. To the mixture was added an additional 10 mg of methyl 2-(2-aminoethylamino)acetate and it was further stirred at rt. After stirring the mixture for an additional 60 h at rt, it was directly purified by flash chromatography using a 0-10% methanol in dichloromethane gradient with 0.1% ammonium hydroxide added to the mixture and a 12 g silica gel column. The fractions containing the expected product were combined and concentrated under reduced pressure to give the product (31 mg, 0.027 mmol, 59.5% yield) as a white foam. LC/MS: m/e 816.5 (M+H)⁺, 2.57 minutes (method 1).

Step 2: To a solution of 2-(2-((1R,3aS,5aR,5bR,7aR,11aS,11bR,13aR,13bS)-9-(4-(methoxycarbonyl)phenyl)-5a,5b,8,8,11a-pentamethyl-1-(3-(2-morpholinoethoxy)prop-1-en-2-yl)-2,3,3a,4,5,5a,5b,6,7,7a,8,11,11a,11b,12,13,13a,13b-octadecahydro-1H-cyclopenta[a]chrysene-3a-carboxamido)ethylamino)acetic acid (30 mg, 0.026 mmol) in 1,4-dioxane (1 mL) was added 1N NaOH (0.2 mL, 0.200 mmol). The mixture was heated to 75° C. for 15 h, then was cooled to rt and purified by prep HPLC (method 1). The fractions containing the expected product were combined and were concentrated under reduced pressure to give the product (10 mg, 0.013 mmol, 48.5% yield) as a white solid. LC/MS: m/e 788.5 (M+H)⁺, 2.01 minutes (method 1). ¹H NMR (500 MHz, acetic acid c/a) δ ppm 8.03 (d, J=8.24 Hz, 2H), 7.29 (d, J=8.24 Hz, 2H), 5.37 (d, J=4.88 Hz, 1H), 5.01 (br. s., 1H), 5.00 (br. s., 1H), 3.29-4.12 (m, 20H), 3.00-3.10 (m, 1H), 2.51-2.60 (m, 1H), 1.09 (s, 3H), 1.08-2.26 (m, 21H), 1.07 (s, 3H), 1.05 (s, 3H), 1.01 (s, 3H), 0.99 (s, 3H).

### Example 18

### Preparation of 4-((1R,3aS,5aR,5bR,7aR,11aS,11bR,13aR,13bS)-3a-(2-(dimethylamino)ethylcarbamoyl)-1-(3-(4-methoxy-N-methyl-4-oxobutanamido)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid

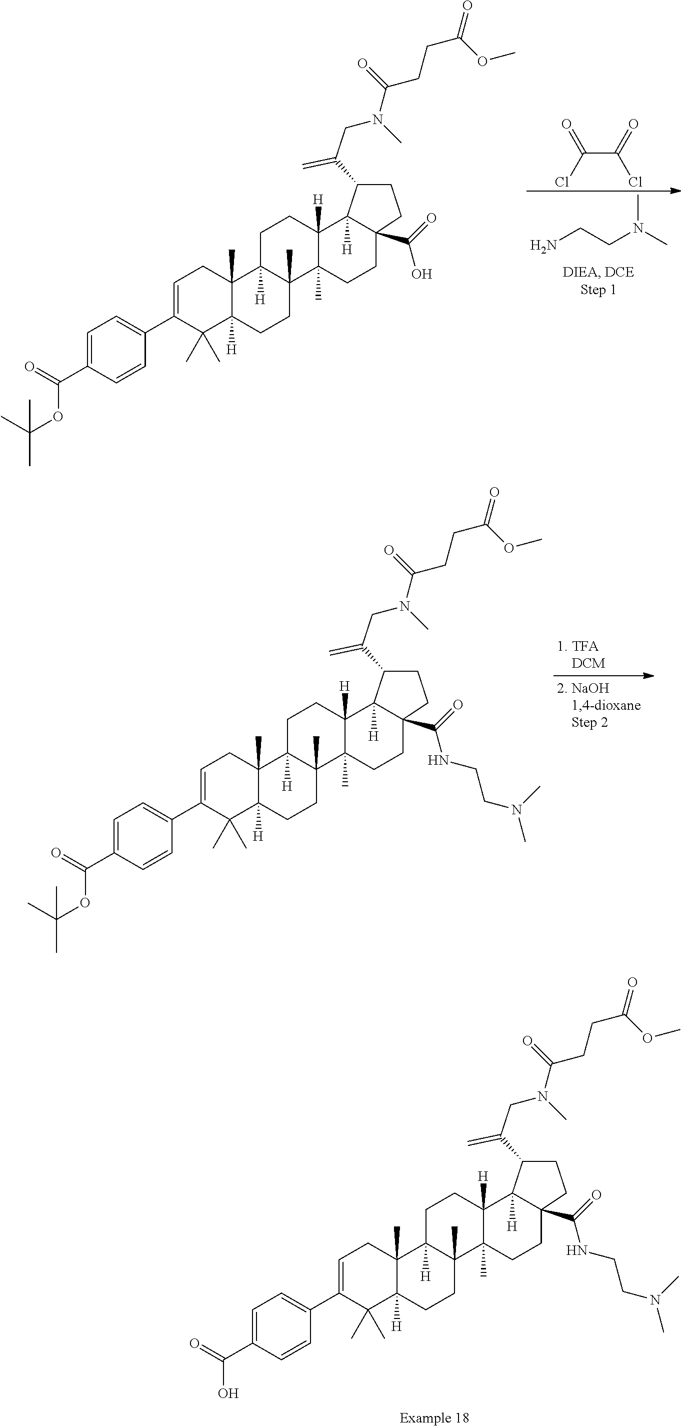

### Step 1: Preparation of tert-butyl 4-((1R,3aS,5aR,5bR,7aR,11aS,11bR,13aR,13bS)-3a-(2-(dimethylamino)ethylcarbamoyl)-1-(3-(4-methoxy-N-methyl-4-oxobutanamido)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate

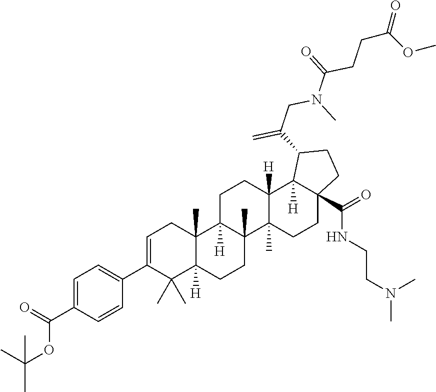

To a vial containing (1R,3aS,5aR,5bR,7aR,11aS,11bR,13aR,13bS)-9-(4-(tert-butoxycarbonyl)phenyl)-1-(3-(4-methoxy-N-methyl-4-oxobutanamido)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid (0.1 g, 0.132 mmol) was added oxalyl chloride (2M in DCM) (2 mL, 4.00 mmol). The solution was stirred at rt for 2.5 and was concentrated under reduced pressure. The residue was dissolved in DCM and concentrated two additional times to remove remaining oxalyl chloride. After drying the residue under vacuum, it was dissolved in DCE (2 mL) and diisopropylethylamine (0.069 mL, 0.396 mmol) was added followed by N1,N1-dimethylethane-1,2-diamine (0.022 mL, 0.198 mmol). The mixture was stirred at rt for 67 h then was diluted with 7 mL of water and was extracted with dichloromethane (3×7 mL). The combined organic layers were dried over sodium sulfate, filtered, and concentrated under reduced pressure. The residue was purified by flash chromatography using a 0-10% methanol in dichloromethane gradient and a 12 g silica gel column. The fractions containing the product were combined and concentrated under reduced pressure to give the product (73.4 mg, 0.089 mmol, 67% yield) as an off-white foam. LC/MS: m/e 828.6 (M+H)⁺, 2.54 minutes (method 1).

Step 2: To a solution of tert-butyl 4-((1R,3aS,5aR,5bR,7aR,11aS,11bR,13aR,13bS)-3a-(2-(dimethylamino)ethylcarbamoyl)-1-(3-(4-methoxy-N-methyl-4-oxobutanamido)prop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (73 mg, 0.088 mmol) in DCM (1 mL) was added TFA (0.1 mL, 1.298 mmol). The mixture was stirred at rt overnight for 16 h at rt then was concentrated under a stream of nitrogen. The residue was diluted with 1,4-dioxane (2 mL) and was heated to 75° C. After 22 h of heating, the mixture was cooled to rt. Then it was acidified with 1N HCl and was heated with a heat gun and was allowed to sit at rt overnight. When no crystals formed, the mixture was purified by prep HPLC (method 1). The fractions containing the product were combined and concentrated under reduced pressure. The residue was repurified a second time using the same HPLC method. The fractions containing the product were combined and concentrated under reduced pressure to give the product (14 mg, 0.018 mmol, 21% yield) as a white solid. LC/MS: m/e 772.5 (M+H)⁺, 2.05 minutes (method 1).

### Examples 19-21

### Preparation of 4-((1R,3aS,5aR,5bR,7aS,9S,11aS,11bR,13aR,13bR)-1-(1-carboxyprop-1-en-2-yl)-3a-((2-(dimethylamino)ethyl)carbamoyl)-5a,5b,8,8,11a-pentamethylicosahydro-1H-cyclopenta[a]chrysen-9-yl)benzoic acid (Example 19), 4-((1S,3aS,5aR,5bR,7aS,9S,11aS,11bR,13aR,13bR)-1-(1-carboxypropan-2-yl)-3a-((2-(dimethylamino)ethyl)carbamoyl)-5a,5b,8,8,11a-pentamethylicosahydro-1H-cyclopenta[a]chrysen-9-yl)benzoic acid, Isomer 1(Example 20), and 4-((1S,3aS,5aR,5bR,7aS,9S,11aS,11bR,13aR,13bR)-1-(1-carboxypropan-2-yl)-3a-((2-(dimethylamino)ethyl)carbamoyl)-5a,5b,8,8,11a-pentamethylicosahydro-1H-cyclopenta[a]chrysen-9-yl)benzoic acid, Isomer 2 (Example 21)

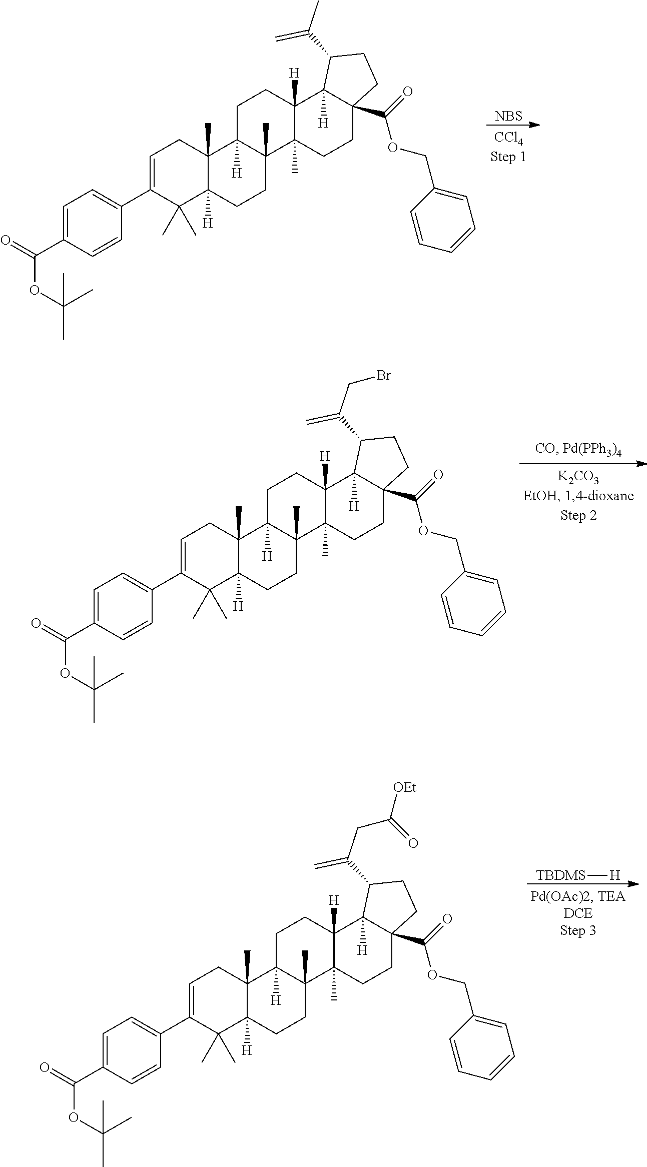

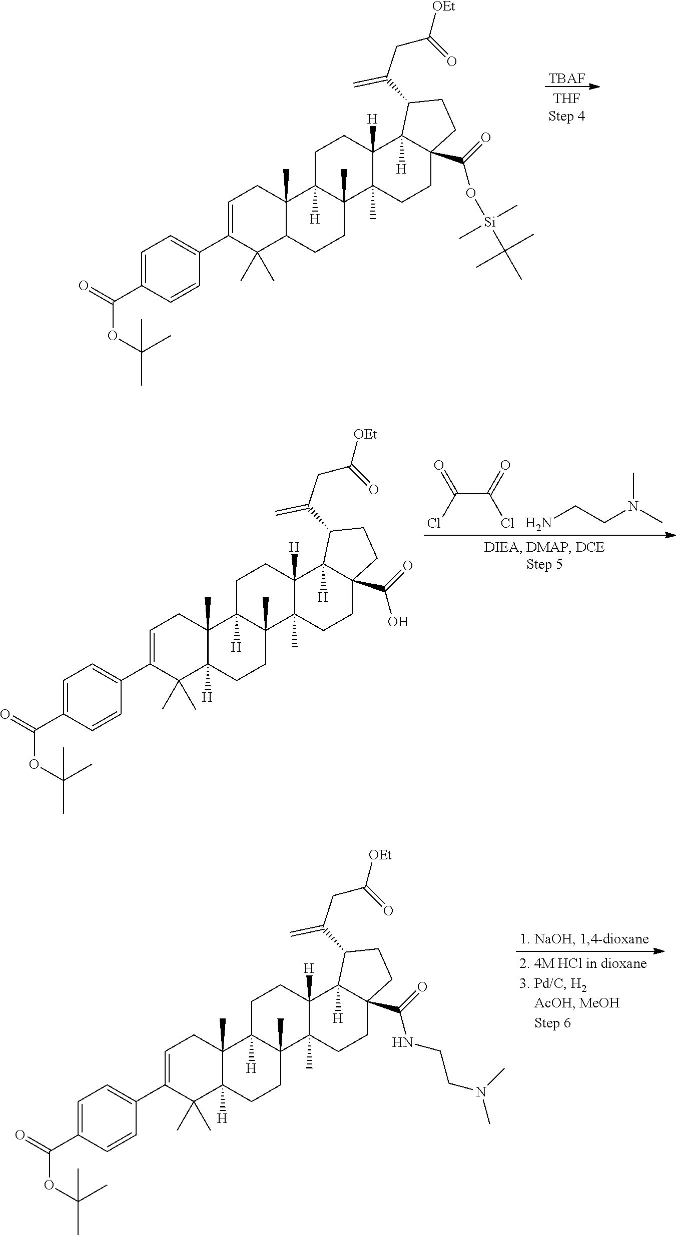

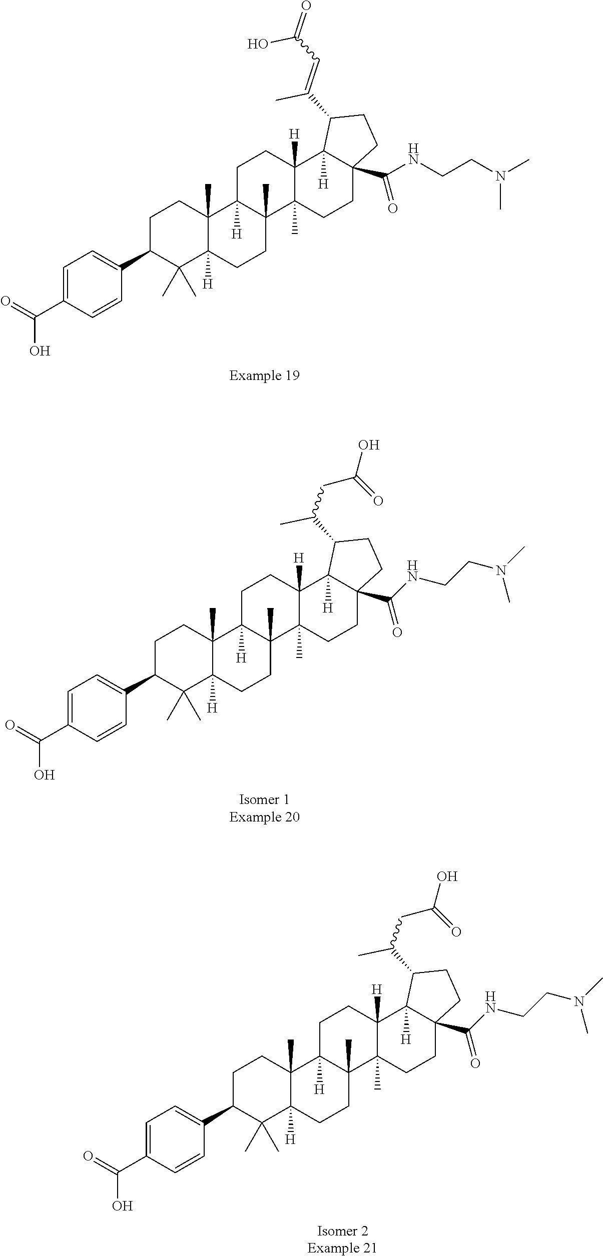

### Step 1: Preparation of (1R,3aS,5aR,5bR,7aR,11aS,11bR,13aR,13bS)-benzyl 1-(3-bromoprop-1-en-2-yl)-9-(4-(tert-butoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylate

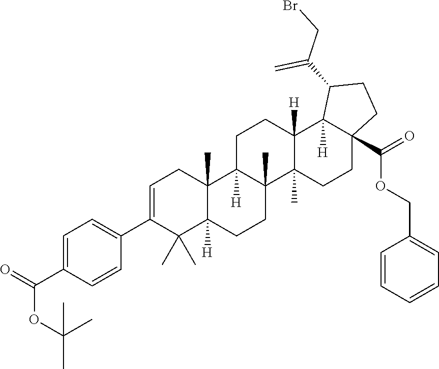

To a solution of (1R,3aS,5aR,5bR,7aR,11aS,11bR,13aR,13bR)-benzyl 9-(4-(tert-butoxycarbonyl)phenyl)-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysene-3a-carboxylate (3.02 g, 4.29 mmol) in CCl₄ (50 mL) was added NBS (0.954 g, 5.36 mmol). The mixture was stirred at rt for 15.5 h then was filtered through a pad of celite to remove the solids and the filtrate was concentrated under reduced pressure. The residue was purified by flash chromatography using a 160 g silica gel column and a 0-10% ethyl acetate in hexanes gradient. The fractions containing the expected product were combined and concentrated under reduced pressure to give 2.22 g of the product as a white foam (70% purity), which was used in the next step with no additional purification. ¹H NMR (500 MHz, CHLOROFORM-d) δ=7.87 (d, J=8.2Hz, 2H), 7.42-7.30 (m, 5H), 7.16 (d, J=7.9 Hz, 2H), 5.26 (d, J=4.9 Hz, 1H), 5.22-5.07 (m, 3H), 5.04 (s, 1H), 4.01-3.96 (m, 2H), 3.09 (td, J=11.1, 4.7 Hz, 1H), 1.58 (s, 9H), 0.98 (s, 3H), 0.94 (s, 3H), 0.90 (s, 6H), 0.80 (s, 3H), 2.36-0.78 (m, 22H).

### Step 2: Preparation of (1R,3aS,5aR,5bR,7aR,11aS,11bR,13aR,13bR)-benzyl 9-(4-(tert-butoxycarbonyl)phenyl)-1-(4-ethoxy-4-oxobut-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylate

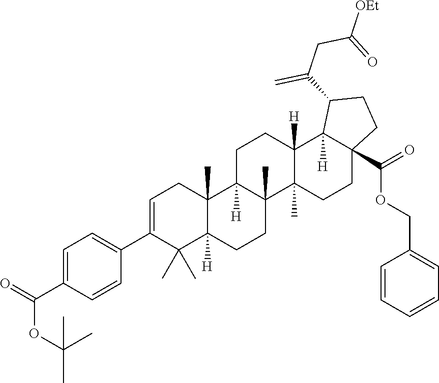

To a pressurizable vessel containing a solution of (1R,3aS,5aR,5bR,7aR,11aS,11bR,13aR,13bS)-benzyl 1-(3-bromoprop-1-en-2-yl)-9-(4-(tert-butoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylate (1.5 g, 1.339 mmol) in 1,4-dioxane (20 mL) and ethanol (10 mL) was added potassium carbonate (0.370 g, 2.68 mmol). The mixture was degassed for 10 minutes by bubbling with nitrogen, and palladium tetrakis (0.077 g, 0.067 mmol) was added. The mixture was evacuated and refilled with nitrogen 3 times, then was filled with carbon monoxide and evacuated two times, then finally filled to 85 psi of carbon monoxide. and was heated to 85° C. in an oil bath. After 24 h of heating, the mixture was cooled to rt, was diluted with 25 mL of water, and was extracted with ethyl acetate (3×25 mL). The combined organic layers were washed with brine, dried over sodium sulfate, filtered, and concentrated under reduced pressure. The residue was purified by flash chromatography using a 0-10% EtOAc in hexanes gradient and a 90 g silica gel column. The fractions containing the expected product were combined and concentrated under reduced pressure to give a major and minor product, the major product (0.591 g, 0.761 mmol, 57% yield), being the title compound. LC/MS: m/e 794.5 (M+18), 4.08 minutes (method 1). ¹H NMR (500 MHz, CHLOROFORM-d) δ=7.87 (d, J=7.9 Hz, 2H), 7.45-7.29 (m, 5H), 7.16 (d, J=7.6 Hz, 2H), 5.26 (d, J=5.5Hz, 1H), 5.20-5.05 (m, 2H), 4.97 (s, 1H), 4.84 (s, 1H), 4.14 (q, J=7.1 Hz, 2H), 3.11-2.95 (m, 3H), 2.37-2.18 (m, 2H), 2.08 (dd, J=17.1, 6.4 Hz, 1H), 2.01-1.85 (m, 2H), 1.58 (s, 9H), 0.97 (s, 3H), 0.94 (s, 3H), 0.90 (s, 6H), 1.72-0.88 (m, 20H), 0.80 (s, 3H).

### Step 3: Preparation of (1R,3aS,5aR,5bR,7aR,11aS,11bR,13aR,13bR)-tert-butyldimethylsilyl 9-(4-(tert-butoxycarbonyl)phenyl)-1-(4-ethoxy-4-oxobut-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylate

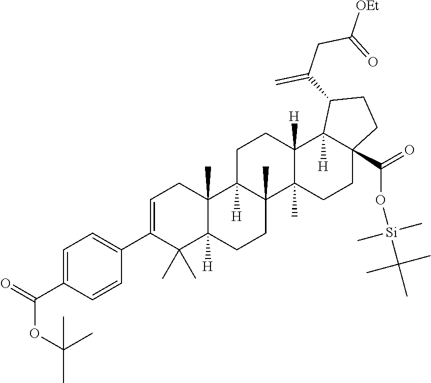

To a solution of (1R,3aS,5aR,5bR,7aR,11aS,11bR,13aR,13bR)-benzyl 9-(4-(tert-butoxycarbonyl)phenyl)-1-(4-ethoxy-4-oxobut-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylate (0.588 g, 0.757 mmol) in DCE (7 mL) was added triethylamine (0.169 mL, 1.211 mmol), tert-butyldimethylsilane (0.251 mL, 1.513 mmol), and palladium(II)acetate (0.042 g, 0.189 mmol). The mixture was flushed with nitrogen and was heated to 60° C. After 5 h of heating, the mixture was cooled to rt, filtered through a pad of celite to remove the solids, and concentrated under reduced pressure. The crude material was used in the next step with no additional purification. ¹H NMR (500 MHz, CHLOROFORM-d) δ=7.86 (d, J=7.9 Hz, 2H), 7.15 (d, J=8.2Hz, 2H), 5.26 (d, J=5.5Hz, 1H), 4.97 (s, 1H), 4.83 (s, 1H), 4.13 (q, J=7.2Hz, 2H), 3.11-2.95 (m, 3H), 2.30-2.21 (m, 2H), 2.08 (dd, J=17.1, 6.4 Hz, 1H), 2.02-1.91 (m, 1H), 1.88-1.79 (m, 1H), 1.57 (s, 9H), 0.94 (s, 9H), 1.71-0.84 (m, 35H), 0.27 (s, 6H).

### Step 4: Preparation of (1R,3aS,5aR,5bR,7aR,11aS,11bR,13aR,13bR)-9-(4-(tert-butoxycarbonyl)phenyl)-1-(4-ethoxy-4-oxobut-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid

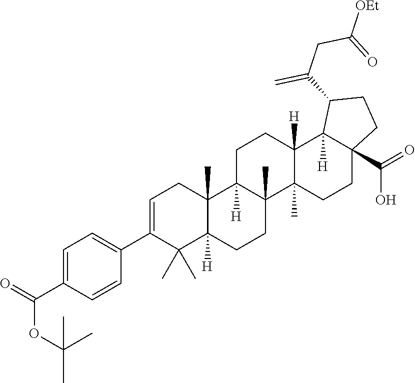

To a solution of (1R,3aS,5aR,5bR,7aR,11aS,11bR,13aR,13bR)-tert-butyldimethylsilyl 9-(4-(tert-butoxycarbonyl)phenyl)-1-(4-ethoxy-4-oxobut-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylate (0.607 g, 0.757 mmol) in THF (10 mL) was added tetrabutylammonium fluoride hydrate (0.317 g, 1.136 mmol). The yellow solution was stirred at rt for 3.5 h then was diluted with 20 mL of water and 10 mL of 1N HCl and was extracted with dichloromethane (3×30 mL). The combined organic layers were washed with brine, dried over sodium sulfate, filtered, and concentrated under reduced pressure. The residue was purified by flash chromatography using a 0-50% ethyl acetate in hexanes gradient and a 40 g silica gel column. The fractions containing the product were combined and concentrated under reduced pressure to give the product (0.485 g, 0.706 mmol, 93% yield) as a white solid. LC/MS: m/e 685.5 (M−H)⁻, 2.90 minutes (method 1). ¹H NMR (500 MHz, CHLOROFORM-d) δ=7.87 (d, J=7.93 Hz, 2H), 7.16 (d, J=8.24 Hz, 2H), 5.27 (d, J=4.58 Hz, 1H), 4.99 (s, 1H), 4.86 (s, 1H), 4.15 (q, J=7.02Hz, 2H), 2.97-3.06 (m, 3H), 2.30 (d, J=12.82Hz, 1H), 2.24 (td, J=12.13, 3.20 Hz, 1H), 2.01-2.14 (m, 2H), 1.97 (dd, J=12.51, 7.93 Hz, 1H), 1.58 (s, 9H), 1.27 (t, J=7.02Hz, 3H), 1.01 (s, 3H), 1.00-1.72 (m, 17H), 0.99 (s, 3H), 0.97 (s, 3H), 0.91 (s, 6H).

### Step 5: Preparation of tert-butyl 4-((1R,3aS,5aR,5bR,7aR,11aS,11bR,13aR,13bR)-3a-(2-(dimethylamino)ethylcarbamoyl)-1-(4-ethoxy-4-oxobut-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate, HC1

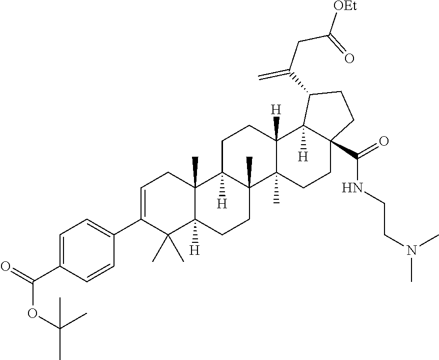

To an oven-dried rb flask containing (1R,3aS,5aR,5bR,7aR,11aS,11bR,13aR,13bR)-9-(4-(tert-butoxycarbonyl)phenyl)-1-(4-ethoxy-4-oxobut-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid (0.15 g, 0.218 mmol) was added oxalyl chloride (2M in DCM) (5 mL, 10.00 mmol). The mixture was stirred at rt for 2.5 h and was concentrated under reduced pressure. The residue was dissolved in DCM and was concentrated two additional times to remove any excess oxalyl chloride. The crude product was dissolved in DCE (2 mL) and diisopropylethylamine (0.114 mL, 0.655 mmol) was added followed by N,N-dimethylethylenediamine (0.036 mL, 0.328 mmol) and DMAP (1 mg, 8.19 μmol). The mixture was stirred at rt for 20 h, then was diluted with water (10 mL) and was extracted with dichloromethane (3×10 mL). The combined organic layers were dried over sodium sulfate, filtered, and concentrated under reduced pressure. The residue was purified by flash chromatography using a 0-10% methanol in dichloromethane gradient and a 12 g silica gel column. The fractions containing the product were combined and concentrated under reduced pressure to give the title compound (0.12 g, 0.151 mmol, 69.3% yield) as a white solid. LC/MS: m/e 757.6 (M+H)⁺, 2.29 minutes (method 6). ¹H NMR (500 MHz, CHLOROFORM-d) δ=12.41 (br. s., 1H), 7.87 (d, J=8.24 Hz, 2H), 7.73 (br. s., 1H), 7.15 (d, J=8.24 Hz, 2H), 5.24-5.28 (m, 1H), 4.98 (s, 1H), 4.83 (s, 1H), 4.14 (q, J=7.32Hz, 2H), 3.62-3.80 (m, 2H), 3.05-3.20 (m, 3H), 2.95-3.03 (m, 2H), 2.81-2.89 (m, 6H), 2.42-2.51 (m, 1H), 2.36 (d, J=14.04 Hz, 1H), 2.09 (dd, J=17.40, 6.41 Hz, 1H), 1.86-1.99 (m, 2H), 1.58 (s, 9H), 1.26 (t, J=7.17 Hz, 3H), 0.99 (s, 3H), 0.95-1.72 (m, 17H), 0.96 (s, 3H), 0.95 (s, 3H), 0.90 (s, 6H).

Step 6: To a solution of tert-butyl 4-((1R,3aS,5aR,5bR,7aR,11aS,11bR,13aR,13bR)-3a-(2-(dimethylamino)ethylcarbamoyl)-1-(4-ethoxy-4-oxobut-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (0.115 g, 0.145 mmol) in 1,4-dioxane (2 mL) was added 1N NaOH (0.725 mL, 0.725 mmol). The mixture was heated to 85° C. for 39 h. NaOH (10N, 0.1 mL) was added to the mixture and it was again heated to 85° C. After 22 h of heating, the mixture was cooled to rt and acidified to pH=1 with 1N HCl. The solids that formed were collected by filtration to give 85 mg of a mixture of products that were used directly in the next step with no additional purification. To a vial containing the mixture of products was added 3 mL of 4N HCl in 1,4 dioxane. The mixture was stirred at rt for 2.5 h, then was concentrated under a stream of nitrogen. The residue was purified by prep HPLC (method 1). The fractions containing a mixture of products were combined and concentrated under reduced pressure. The mixture of products was dissolved in acetic acid (2 mL) and methanol (4 mL) was degassed with nitrogen and 20 mg of 10% Pd/C was added and the mixture. The mixture was stirred under 1ATM of H₂ for 3 h then an additional 100 mg of Pd/C were added and mixture was stirred under 1ATM of H₂. After 21 h of stirring, the mixture was filtered through a plug of celite and was concentrated under reduced pressure. The residue was purified by prep HPLC (method 1). Three main peaks were separated in the first prep HPLC purification. The fractions containing mono-hydrogenated product were combined, concentrated, and repurified by prep HPLC (method 10). Each of the other two products was concentrated to give two diastereomers of the bis-hydroxylated product.

### Example 19

### (Isolate 1) 4-((1R,3aS,5aR,5bR,7aS,9S,11aS,11bR,13aR,13bR)-1-(1-carboxyprop-1-en-2-yl)-3a-((2-(dimethylamino)ethyl)carbamoyl)-5a,5b,8,8,11a-pentamethylicosahydro-1H-cyclopenta[a]chrysen-9-yl)benzoic acid (4.0 mg, 0.006 mmol, 4% yield)

LC/MS: m/e 675.5 (M+H)⁺, 2.05 minutes (method 1). ¹H NMR (400 MHz, CHLOROFORM-d with three drops of METHANOL-d₄ to solubilize (referenced to chloroform peak at 7.27 ppm)) δ=7.81 (d, J=8.3 Hz, 2H), 7.12 (d, J=8.3 Hz, 2H), 5.66 (s, 1H), 3.35-3.29 (m, 2H), 3.12 (td, J=10.7, 3.9 Hz, 1H), 2.62 (t, J=5.9 Hz, 2H), 2.40 (s, 6H), 2.00 (s, 3H), 0.89 (s, 3H), 0.86 (s, 3H), 0.84 (br. s., 3H), 2.49-0.71 (m, 25H), 0.65 (s, 3H), 0.59 (s, 3H).

### Example 20

### (Isolate 2) 4-((1S,3aS,5aR,5bR,7aS,9S,11aS,11bR,13aR,13bR)-1-(1-carboxypropan-2-yl)-3a-((2-(dimethylamino)ethyl)carbamoyl)-5a,5b,8,8,11a-pentamethylicosahydro-1H-cyclopenta[a]chrysen-9-yl)benzoic acid, Isomer 1 (6.5 mg, 0.010 mmol, 7% yield)

LC/MS: m/e 677.6 (M+H)⁺, 2.19 minutes (method 1).

¹H NMR (500 MHz, CHLOROFORM-d with three drops of METHANOL-d₄ to solubilize (referenced to chloroform peak at 7.27 ppm))=7.82 (d, J=7.9 Hz, 2H), 7.11 (d, J=8.2 Hz, 2H), 3.37 (t, J=5.2Hz, 2H), 2.78-2.70 (m, 2H), 2.50 (s, 6H), 2.39-2.24 (m, 3H), 2.16-2.08 (m, 2H), 2.07-1.94 (m, 3H), 1.76-1.60 (m, 4H), 0.88 (s, 3H), 0.84 (br. s., 6H), 0.74 (d, J=6.1 Hz, 3H), 1.51-0.70 (m, 17H), 0.63 (s, 3H), 0.59 (s, 3H).

### Example 21

### (Isolate 3) 4-((1S,3aS,5aR,5bR,7aS,9S,11aS,11bR,13aR,13bR)-1-(1-carboxypropan-2-yl)-3a-((2-(dimethylamino)ethyl)carbamoyl)-5a,5b,8,8,11a-pentamethylicosahydro-1H-cyclopenta[a]chrysen-9-yl)benzoic acid, Isomer 2 (5.5 mg, 0.008 mmol, 5.5% yield)

LC/MS: m/e 677.5 (M+H)⁺, 2.04 minutes (method 1). ¹H NMR (500 MHz, CHLOROFORM-d with three drops of METHANOL-d₄ to solubilize (referenced to chloroform peak at 7.27 ppm)) δ=7.89 (d, J=8.2Hz, 2H), 7.19 (d, J=8.5Hz, 2H), 3.53-3.46 (m, 2H), 2.96-2.88 (m, 2H), 2.66 (br. s., 6H), 2.48-2.31 (m, 4H), 2.20-0.74 (m, 37H), 0.71 (s, 3H), 0.65 (s, 3H).

### Example 22

### Preparation of 4-((1R,3aS,5aR,5bR,7aR,11aS,11bR,13aR,13bS)-3a-((2-(1,1-dioxidothiomorpholino)ethyl)amino)-1-(2-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)cyclohex-3-enecarboxylic acid

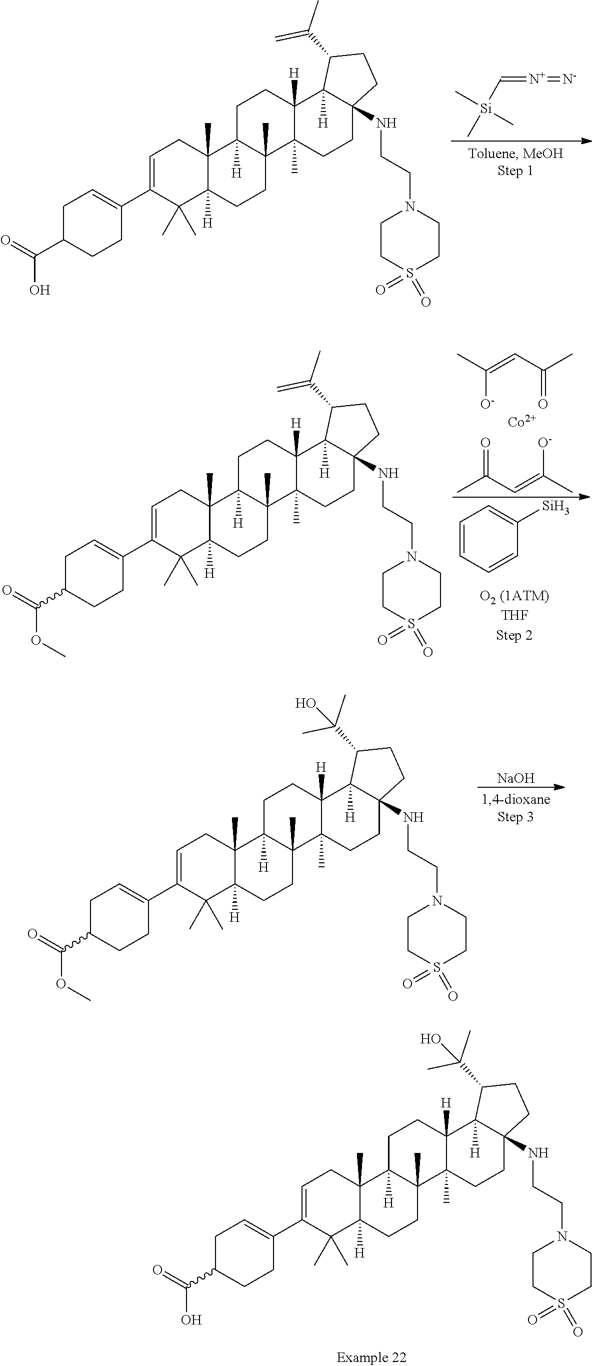

### Step 1: Preparation of methyl 4-((1R,3aS,5aR,5bR,7aR,11aS,11bR,13aR,13bR)-3a-((2-(1,1-dioxidothiomorpholino)ethyl)amino)-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysen-9-yl)cyclohex-3-enecarboxylate

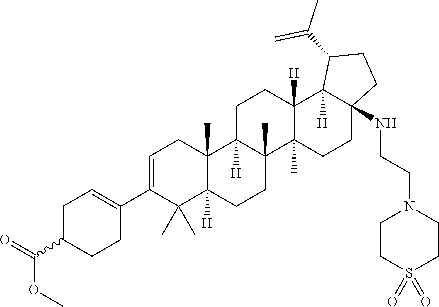

A solution of 4-((1R,3aS,5aR,5bR,7aR,11aS,11bR,13aR,13bR)-3a-((2-(1,1-dioxidothiomorpholino)ethyl)amino)-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysen-9-yl)cyclohex-3-enecarboxylic acid, prepared as described in WO13123019, (0.1 g, 0.144 mmol) in toluene (2 mL) and methanol (0.5 mL) was cooled to 0° C. To the solution was added TMS-diazomethane (2M in ether) (0.086 mL, 0.173 mmol) dropwise. After gas evolution ceased, the mixture was warmed to rt and the yellow solution was stirred at rt for 2 h. The mixture was made acidic by carefully adding 1 mL of acetic acid then was concentrated under reduced pressure. The residue was diluted with sat. aq. sodium bicarbonate (15 mL) and was extracted with dichloromethane (3×10 mL). The combined organic layers were dried over sodium sulfate, filtered, and concentrated under reduced pressure to give the title product (0.086 g, 0.121 mmol, 84% yield) as an off-white foam. ¹H NMR (400 MHz, CHLOROFORM-d) δ=5.35 (br. s., 1H), 5.19 (d, J=5.0 Hz, 1H), 4.71 (br. s., 1H), 4.59 (br. s., 1H), 3.69 (s, 3H), 3.14-2.97 (m, 8H), 2.74-2.41 (m, 6H), 2.34-2.27 (m, 2H), 2.22-2.13 (m, 2H), 1.69 (s, 3H), 2.06-0.78 (m, 40H).

### Step 2: Preparation of methyl 4-((1R,3aS,5aR,5bR,7aR,11aS,11bR,13aR,13bS)-3a-((2-(1,1-dioxidothiomorpholino)ethyl)amino)-1-(2-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)cyclohex-3-enecarboxylate

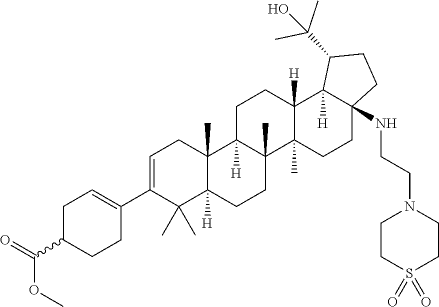

To a flask containing methyl 4-((1R,3aS,5aR,5bR,7aR,11aS,11bR,13aR,13bR)-3a-((2-(1,1-dioxidothiomorpholino)ethyl)amino)-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysen-9-yl)cyclohex-3-enecarboxylate (0.086 g, 0.121 mmol) was added acetylacetone cobalt(II) salt (0.062 g, 0.243 mmol). The mixture was diluted with THF (2 mL) and phenylsilane (0.060 mL, 0.485 mmol) was added. The mixture was purged with nitrogen, then was put under a balloon of oxygen. After 1.5 h the mixture was diluted with dichloromethane and was filtered through a 4 g silica gel column (washed with 10% MeOH in DCM). The filtrate was concentrated under reduced pressure. The residue was repurified using a 0-8% MeOH in dichloromethane gradient and a 12 g silica gel column.

The fractions containing the major isolate were combined and concentrated under reduced pressure to give 56 mg of a light-green solid which was used in the next step with no additional purification.

Step 3: To a solution of methyl 4-((1R,3aS,5aR,5bR,7aR,11aS,11bR,13aR,13bS)-3a-((2-(1,1-dioxidothiomorpholino)ethyl)amino)-1-(2-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)cyclohex-3-enecarboxylate (0.056 g, 0.077 mmol) in 1,4-dioxane (2 mL) was added NaOH (1N) (0.385 mL, 0.385 mmol) and the mixture was heated to 70° C. After heating the mixture for 18 h, it was cooled to rt, and was purified by prep HPLC (method 21). The fractions containing the expected product were combined and concentrated under reduced pressure to give 4-((1R,3aS,5aR,5bR,7aR,11aS,11bR,13aR,13bS)-3a-((2-(1,1-dioxidothiomorpholino)ethyl)amino)-1-(2-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)cyclohex-3-enecarboxylic acid, TFA (9.2 mg, 11.1 mmol, 14% yield) as a white solid. LC/MS: m/e 713.6 (M+H)⁺, 1.44 minutes (method 6). ¹H NMR (400 MHz, Acetic) δ=5.39 (br. s., 1H), 5.24 (d, J=5.8 Hz, 1H), 3.38-3.01 (m, 12H), 2.65-2.55 (m, 1H), 1.28 (s, 3H), 1.26 (s, 3H), 1.22 (s, 3H), 1.12 (s, 3H), 2.40-0.84 (m, 38H).

### Example 23

### Preparation of 4-((1R,3aS,5aR,5bR,7aR,11aS,11bR,13aR,13bS)-3a-((2-(2-oxa-5-azabicyclo[2.2.1]heptan-5-yl)ethyl)amino)-1-(2-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)cyclohex-3-enecarboxylic acid

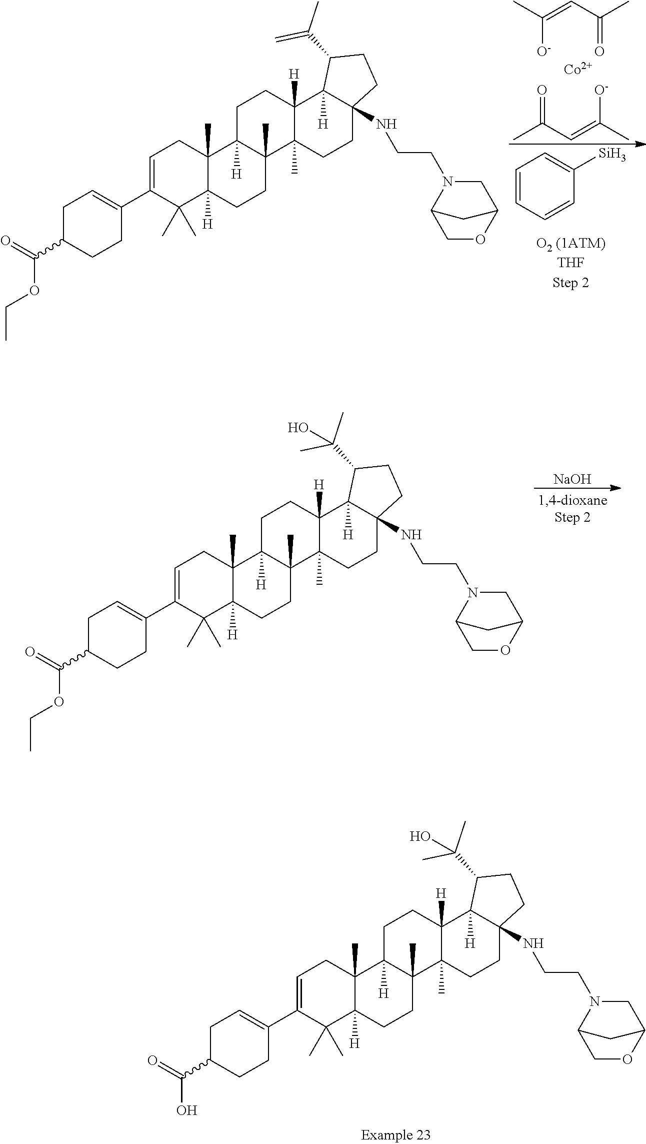

### Step 1: Preparation of ethyl 4-((1R,3aS,5aR,5bR,7aR,11aS,11bR,13aR,13bS)-3a-((2-(2-oxa-5-azabicyclo[2.2.1]heptan-5-yl)ethyl)amino)-1-(2-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)cyclohex-3-enecarboxylate

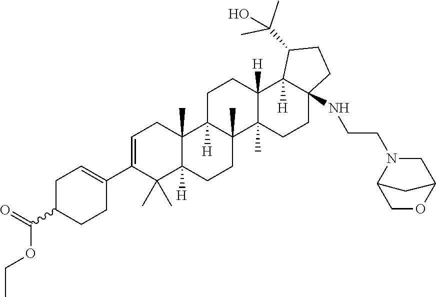

To a flask containing ethyl 4-((1R,3aS,5aR,5bR,7aR,11aS,11bR,13aR,13bR)-3a-((2-(2-oxa-5-azabicyclo[2.2.1]heptan-5-yl)ethyl)amino)-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysen-9-yl)cyclohex-3-enecarboxylate (46.5 mg, 0.068 mmol) prepared as described in WO13169578, was added acetylacetone cobalt(II) salt (34.8 mg, 0.135 mmol). The mixture was diluted with THF (2 mL) and phenylsilane (0.033 mL, 0.271 mmol) was added. The mixture was purged with nitrogen, then was put under a balloon of oxygen. After 4.5 h of stirring the mixture was diluted with dichloromethane and was directly purified using a 0-10% MeOH in dichloromethane gradient and a 12 g silica gel column. The fractions containing the two major isolates were concentrated under reduced pressure to give the title compound (7.5 mg, 0.011 mmol, 16% yield) as a light-green solid. LC/MS: m/e 705.7 (M+H)⁺, 1.76 minutes (method 6).

Step 2: To a solution of ethyl 4-((1R,3aS,5aR,5bR,7aR,11aS,11bR,13aR,13bS)-3a-((2-(2-oxa-5-azabicyclo[2.2.1]heptan-5-yl)ethyl)amino)-1-(2-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)cyclohex-3-enecarboxylate (7.5 mg, 11 mmol) in 1,4-dioxane (0.5 mL) was added NaOH (1N) (0.074 mL, 0.074 mmol). The mixture was warmed to 75° C. for three hours, then was cooled to rt and purified by prep HPLC (method 22). The fractions containing the expected product were combined and concentrated under reduced pressure to give of 4-((1R,3aS,5aR,5bR,7aR,11aS,11bR,13aR,13bS)-3a-((2-(2-oxa-5-azabicyclo[2.2.1]heptan-5-yl)ethyl)amino)-1-(2-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)cyclohex-3-enecarboxylic acid, TFA (1.8 mg, 2.3 mmol, 22% yield) as a clear, colorless film. LC/MS: m/e 677.7 (M+H)⁺, 1.40 minutes (method 6).

¹H NMR (500 MHz, Acetic) δ=5.39 (br. s., 1H), 5.23 (d, J=6.3 Hz, 1H), 4.74 (s, 1H), 4.57 (s, 1H), 4.23-4.16 (m, 1H), 3.98-3.44 (m, 11H), 2.64-2.56 (m, 1H), 2.41-0.74 (m, 48H).

### Example A1

### Preparation of 4-((1R,3aS,5aR,5bR,7aR,11aS,11bR,13aR,13bS)-3a-((tert-butoxycarbonyl)amino)-1-(3-hydroxyprop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoic acid

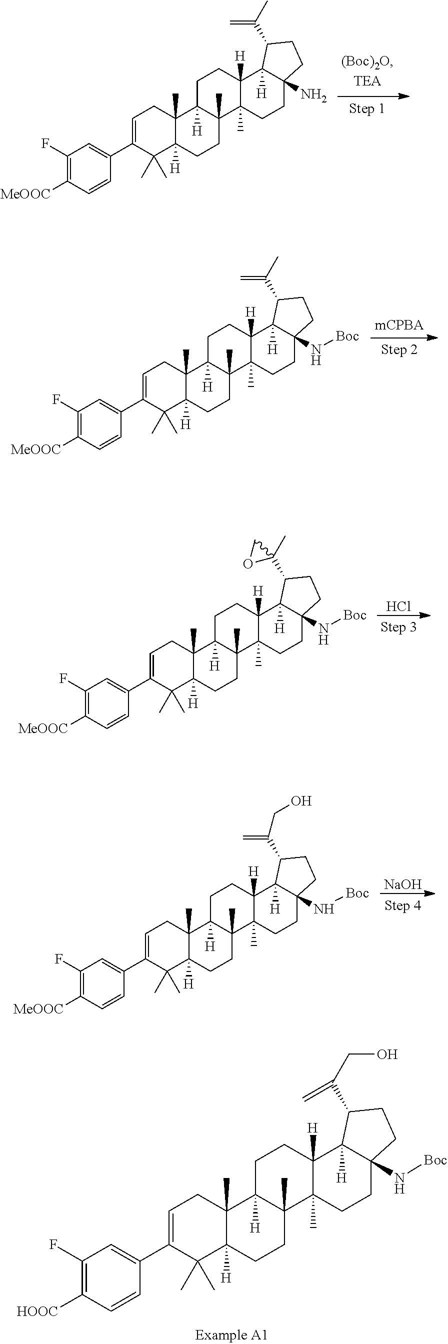

### Step 1. Preparation of methyl 4-((1R,3aS,5aR,5bR,7aR,11aS,11bR,13aR,13bR)-3a-((tert-butoxycarbonyl)amino)-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysen-9-yl)-2-fluorobenzoate

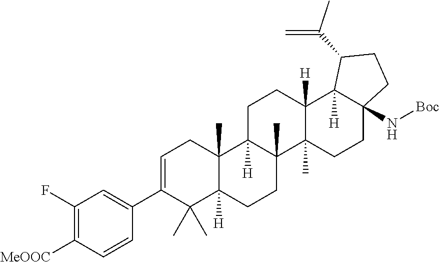

To a solution of methyl 4-((1R,3aS,5aR,5bR,7aR,11aS,11bR,13aR,13bR)-3a-amino-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysen-9-yl)-2-fluorobenzoate prepared as described in WO201206190 (1.5 g, 2.67 mmol) and triethylamine (0.744 mL, 5.34 mmol) in THF (30 mL) was added di-tert-butyl dicarbonate (0.930 mL, 4.00 mmol). The reaction mixture was stirred for 15 hours at room temperature. The reaction mixture was then quenched with distilled water (15 mL), extracted with dichloromethane (2×15 mL). The organic phases were combined, dried over sodium sulfate, filtered and concentrated under reduced pressure to provide the desired product as colorless oil (1.8 g, 100%). LCMS: m/e 662.42 (M+H)⁺, 3.39 min (method 4).

### Step 2. Preparation of methyl 4-((1R,3aS,5aR,5bR,7aR,11aS,11bR,13aR,13bS)-3a-((tert-butoxycarbonyl)amino)-5a,5b,8,8,11a-pentamethyl-1-(2-methyloxiran-2-yl)-2,3,3a,4,5,5a,5b,6,7,7a,8,11,11a,11b,12,13,13a,13b-octadecahydro-1H-cyclopenta[a]chrysen-9-yl)-2-fluorobenzoate

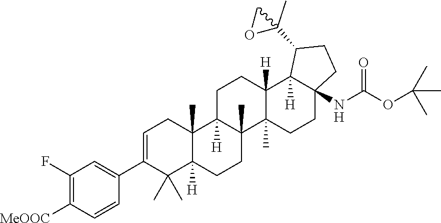

To a solution of methyl 4-((1R,3aS,5aR,5bR,7aR,11aS,11bR,13aR,13bR)-3a-((tert-butoxycarbonyl)amino)-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysen-9-yl)-2-fluorobenzoate (1800 mg, 2.72 mmol) in dichloromethane (20 mL) at 0° C. was added 3-chlorobenzoperoxoic acid (670 mg, 2.99 mmol). The reaction mixture was stirred for 2 hours at 0° C. and then warmed up to room temperature for 2 hours. The reaction mixture was then quenched with saturated aqueous Na₂S₂O₃ (25 mL), extracted with dichloromethane (2×30 mL), the organic phases were combined, dried over sodium sulfate, filtered and concentrated under reduced pressure to provide the desired product as colorless oil. the residue was purified on silica gel with 0-30% ethyl acetate/hexanes to provide the desired product as white solid (1.0 g, 54%). LCMS: m/e 678.39 (M+H)⁺, 3.69 min (method 4).

### Step 3. Preparation of methyl 4-((1R,3aS,5aR,5bR,7aR,11aS,11bR,13aR,13bS)-3a-((tert-butoxycarbonyl)amino)-1-(3-hydroxyprop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoate

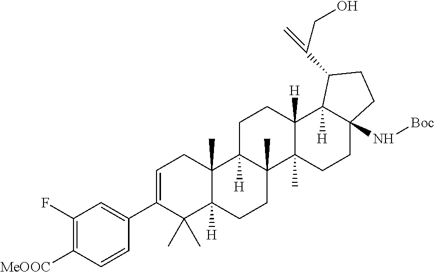

A mixture of methyl 4-((1R,3aS,5aR,5bR,7aR,11aS,11bR,13aR,13bS)-3a-((tert-butoxycarbonyl)amino)-5a,5b,8,8,11a-pentamethyl-1-(2-methyloxiran-2-yl)-2,3,3a,4,5,5a,5b,6,7,7a,8,11,11a,11b,12,13,13a,13b-octadecahydro-1H-cyclopenta[a]chrysen-9-yl)-2-fluorobenzoate (200 mg, 0.295 mmol) and HCl (0.516 mL, 2.065 mmol) in THF (2 mL) was stirred for 2 hours until LCMS indicated starting material was consumed. The reaction mixture was concentrated under reduced pressure, the residue was dissolved in acetonitrile (1 mL) and purified by HPLC to provide the desired product as white solid (30 mg, 15%). LCMS: m/e 578.5 (M+H)⁺, 1.93 min (method 6).

Step 4: A mixture of methyl 4-((1R,3aS,5aR,5bR,7aR,11aS,11bR,13aR,13bS)-3a-((tert-butoxycarbonyl)amino)-1-(3-hydroxyprop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoate (30 mg, 0.044 mmol) and 1 N NaOH (0.443 mL, 0.443 mmol) in dioxane (1 mL) was heated up at 78° C. for 3 hours. The reaction mixture was precipitated after cooling down to rt. The white solid was filtered and washed with water (2 mL) and acetonitrile (2 mL) to provide the desired product (19 mg, 61%). LCMS: m/e 664.5 (M+H)⁺, 2.55 min (method 6). ¹H NMR (400 MHz, Acetone) δ 7.90 (t, J=7.9 Hz, 1H), 7.10 (dd, J=8.0, 1.5Hz, 1H), 7.02 (dd, J=11.9, 1.4 Hz, 1H), 5.38 (dd, J=6.1, 1.9 Hz, 1H), 5.01 (d, J=1.8 Hz, 1H), 4.88 (s, 1H), 4.10 (s, 2H), 2.72-1.08 (m, 23H), 1.42 (s, 9H), 1.15 (s, 3H), 1.07 (s, 3H), 1.04 (s, 3H), 1.01 (s, 3H), 0.99 (s, 3H).

### Example A2

### Preparation of 4-41R,3aS,5aR,5bR,7aR,11aS,11bR,13aR,13bS)-3a-amino-1-(3-hydroxyprop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoic acid

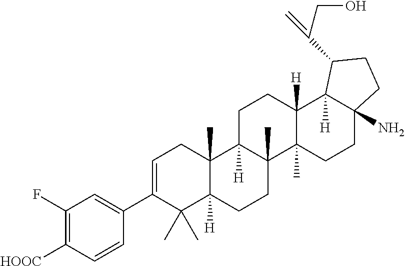

A mixture of methyl 4-((1R,3aS,5aR,5bR,7aR,11aS,11bR,13aR,13bS)-3a-amino-1-(3-hydroxyprop-1-en-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoate (6 mg, 10.38 mmol) and 1 N NaOH (0.104 mL, 0.104 mmol) was heated up at 78° C. for 3 hours. The reaction mixture was cooled to room temperature, then neutralized with 1 N HCl to pH˜4-6, the white precipitate was filtered and washed with distilled water to provide the desired product as white solid (4 mg, 65%). LCMS: m/e 564.18 (M+H)⁺, 2.44 min (method 4). ¹H NMR (400 MHz, Acetic) δ 7.92 (t, J=7.9 Hz, 1H), 7.04 (d, J=8.3 Hz, 1H), 6.98 (d, J=12.0 Hz, 1H), 5.38 (d, J=4.8 Hz, 1H), 5.08 (s, 1H), 5.00 (s, 1H), 4.21 (s, 2H), 2.78-2.60 (m, 1H), 2.33-1.13 (m, 22H), 1.16 (s, 3H), 1.08 (s, 3H), 1.03 (s, 3H), 0.99 (s, 3H), 0.97 (s, 3H).

### Example A3

### Preparation of 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(2-methyloxiran-2-yl)-2,3,3a,4,5,5a,5b,6,7,7a,8,11,11a,11b,12,13,13a,13b-octadecahydro-1H-cyclopenta[a]chrysen-9-yl)-2-fluorobenzoic acid

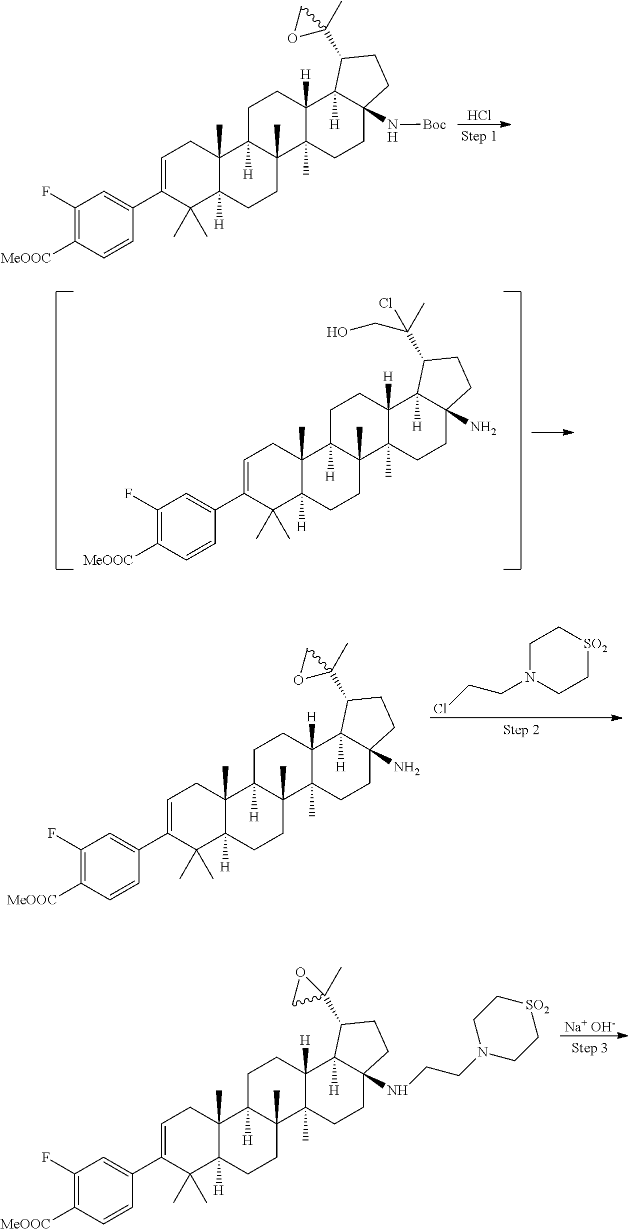

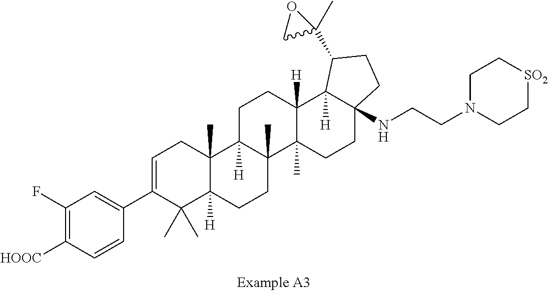

### Step 1. Preparation of methyl 4-((1R,3aS,5aR,5bR,7aR,11aS,11bR,13aR,13bS)-3a-amino-5a,5b,8,8,11a-pentamethyl-1-(2-methyloxiran-2-yl)-2,3,3a,4,5,5a,5b,6,7,7a,8,11,11a,11b,12,13,13a,13b-octadecahydro-1H-cyclopenta[a]chrysen-9-yl)-2-fluorobenzoate

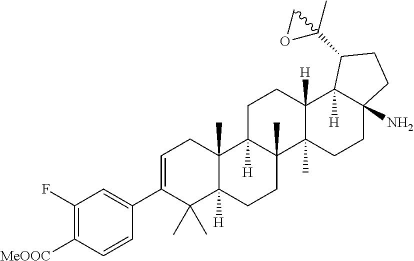

A mixture of methyl 4-((1R,3aS,5aR,5bR,7aR,11aS,11bR,13aR,13bS)-3a-((tert-butoxycarbonyl)amino)-5a,5b,8,8,11a-pentamethyl-1-(2-methyloxiran-2-yl)-2,3,3a,4,5,5a,5b,6,7,7a,8,11,11a,11b,12,13,13a,13b-octadecahydro-1H-cyclopenta[a]chrysen-9-yl)-2-fluorobenzoate (150 mg, 0.221 mmol) and hydrogen chloride (1.106 mL, 4.43 mmol) in THF (3 mL) was stirred for 30 hours. The reaction was stopped and quenched with distilled water (4 mL), extracted with dichloromethane (3×2 mL), the combined organic phases were dried over sodium sulfate, filtered and concentrated under reduced pressure to provide the title compound as colorless oil (100 mg, 78%). LCMS: m/e 578.2 (M+H)⁺, 1.98 min (method 4).

### Step 2. Preparation of methyl 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(2-methyloxiran-2-yl)-2,3,3a,4,5,5a,5b,6,7,7a,8,11,11a,11b,12,13,13a,13b-octadecahydro-1H-cyclopenta[a]chrysen-9-yl)-2-fluorobenzoate

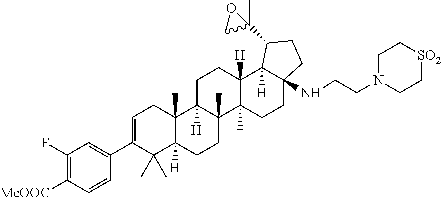

A mixture of methyl 4-((1R,3aS,5aR,5bR,7aR,11aS,11bR,13aR,13bS)-3a-amino-5a,5b,8,8,11a-pentamethyl-1-(2-methyloxiran-2-yl)-2,3,3a,4,5,5a,5b,6,7,7a,8,11,11a,11b,12,13,13a,13b-octadecahydro-1H-cyclopenta[a]chrysen-9-yl)-2-fluorobenzoate (68.4 mg, 0.346 mmol), potassium phosphate (110 mg, 0.519 mmol) and potassium iodide (28.7 mg, 0.173 mmol) in acetonitrile (1 mL) was heated up at 120° C. for 2 hours. The reaction mixture was quenched with distilled water (3 mL), extracted with dichloromethane (3×2 mL), the combined organic phases were dried over Na₂SO₄, filtered and concentrated under reduced pressure to provide the crude as yellow oil. the crude was purified by prep HPLC to provide the title as colorless oil (20 mg, 16%). LCMS: m/e 739.55 (M+H)⁺, 2.09 min (method 6).

Step 3. A mixture of methyl 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(2-methyloxiran-2-yl)-2,3,3a,4,5,5a,5b,6,7,7a,8,11,11a,11b,12,13,13a,13b-octadecahydro-1H-cyclopenta[a]chrysen-9-yl)-2-fluorobenzoate (20 mg, 0.027 mmol) and 1 N NaOH (0.271 mL, 0.271 mmol) in dioxane (1 mL) were heated up at 78° C. for 3 hours. The reaction mixture was filtered and purified by prep HPLC to provide 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(2-methyloxiran-2-yl)-2,3,3a,4,5,5a,5b,6,7,7a,8,11,11a,11b,12,13,13a,13b-octadecahydro-1H-cyclopenta[a]chrysen-9-yl)-2-fluorobenzoic acid as colorless oil (10 mg, 50%). LCMS: m/e 725.55 (M+H)⁺, 2.67 min (method 6).¹H NMR (400 MHz, ACETONITRILE-d₃) δ 7.81 (t, J=8.0 Hz, 1H), 7.04 (dd, J=8.0, 1.3 Hz, 1H), 6.99 (dd, J=12.2, 1.4 Hz, 1H), 5.33 (dd, J=6.1, 1.9 Hz, 1H), 3.28-2.54 (m, 15H), 2.21-0.98 (m, 22H), 1.18 (s, 3H), 1.07-0.99 (m, 9H), 0.95 (s, 3H), 0.93 (s, 3H).

### Example A4 and Example A5

### Preparation of 4-((1R,3aS,5aR,5bR,7aR,11aS,11bR,13aR,13bR)-3a-((2-(1,1-dioxidothiomorpholino)ethyl)amino)-1-(1-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoic acid (diastereoisomer 1 and diastereoisomer 2)

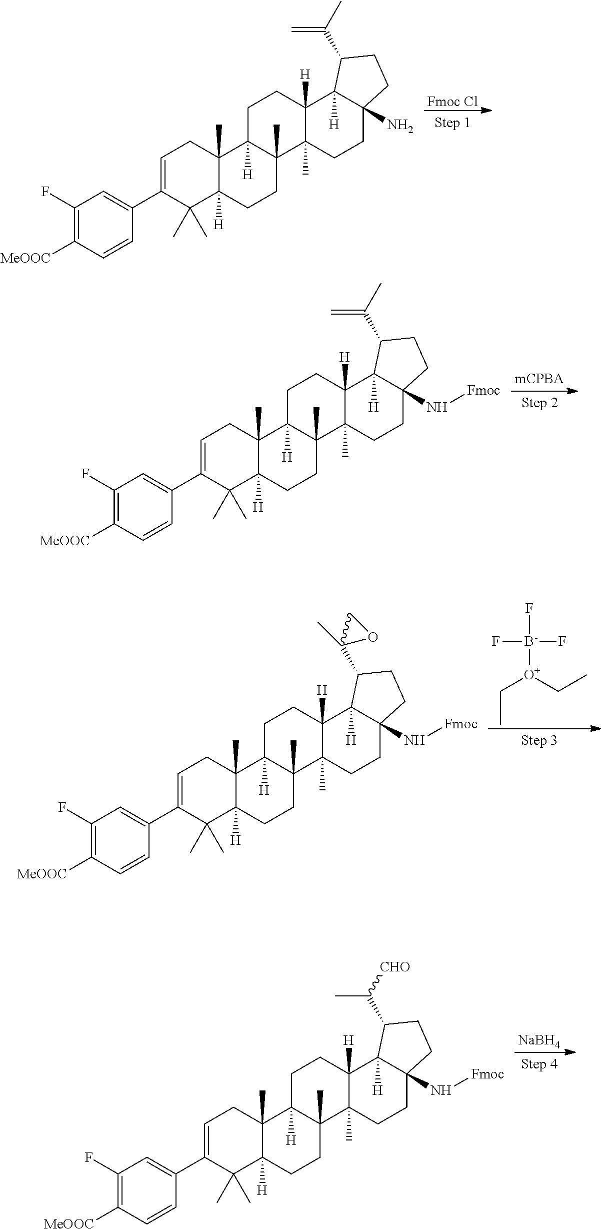

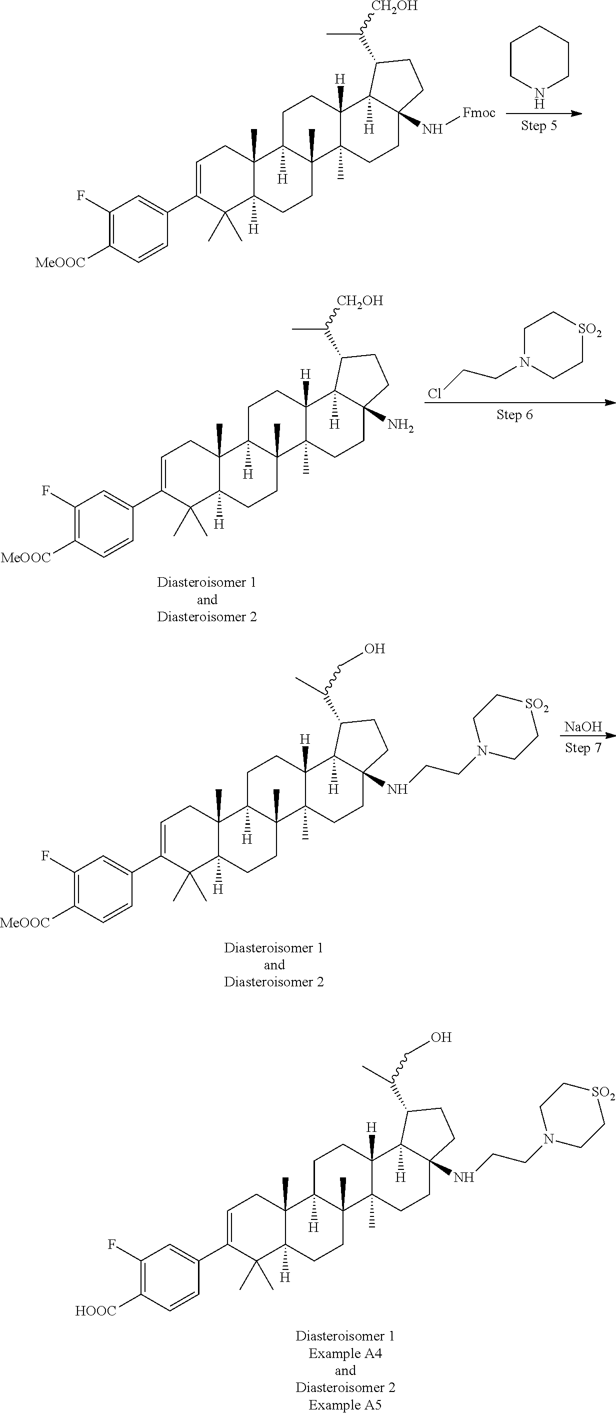

### Step 1. Preparation of methyl 4-((1R,3aS,5aR,5bR,7aR,11aS,11bR,13aR,13bR)-3a-((((9H-fluoren-9-yl)methoxy)carbonyl)amino)-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysen-9-yl)-2-fluorobenzoate

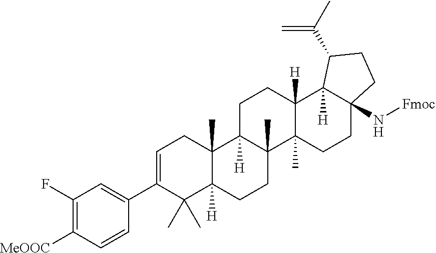

To a mixture of methyl 4-((1R,3aS,5aR,5bR,7aR,11aS,11bR,13aR,13bR)-3α-amino-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysen-9-yl)-2-fluorobenzoate (2000 mg, 3.56 mmol) and sodium carbonate (1509 mg, 14.24 mmol) in THF (50 mL) and water (50 mL) at room temperature was added (9H-fluoren-9-yl)methyl carbonochloridate (1105 mg, 4.27 mmol) in THF (5 mL). The reaction mixture was stirred for 2 hours, quenched with distilled water (10 mL) and extracted with ethyl acetate (3×6 mL), the combined organic phases were dried over sodium sulfate, filtered and concentrated under reduced pressure. The crude material was purified using silica gel with 0-30% ethyl acetate/hexanes as the mobile phase to provide the desired product as a white solid (2.3 g, 81%). LCMS: m/e 784.3 (M+H)⁺, 4.7 min (method 4).

### Step 2. Preparation of methyl 4-((1R,3aS,5aR,5bR,7aR,11aS,11bR,13aR,13bS)-3a-((((9H-fluoren-9-yl)methoxy)carbonyl)amino)-5a,5b,8,8,11a-pentamethyl-1-(2-methyloxiran-2-yl)-2,3,3a,4,5,5a,5b,6,7,7a,8,11,11a,11b,12,13,13a,13b-octadecahydro-1H-cyclopenta[a]chrysen-9-yl)-2-fluorobenzoate

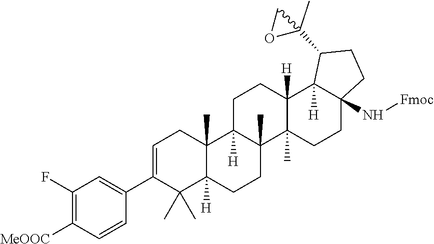

To a solution of methyl 4-((1R,3aS,5aR,5bR,7aR,11aS,11bR,13aR,13bR)-3a-((((9H-fluoren-9-yl)methoxy)carbonyl)amino)-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysen-9-yl)-2-fluorobenzoate (950 mg, 1.212 mmol) in dichloromethane (4 mL) at 0° C. was added 3-chlorobenzoperoxoic acid (326 mg, 1.454 mmol). The reaction mixture was stirred for 18 hours, quenched with sat. Na₂S₂O₃(25 mL) and extracted with dichloromethane (2×30 mL). The combined organic phases were washed with saturated aqueous solution of sodium bicarbonate, dried over sodium sulfate, filtered and concentrated under reduced pressure. The resulting yellow oil was purified using silica gel with 0-30% ethyl acetate/hexanes as the mobile phase to provide the desired product as a white solid (780 mg, 80%). LCMS: m/e 800.29 (M+H)⁺, 3.21 min (method 4).

### Step 3. Preparation methyl 4-((1R,3aS,5aR,5bR,7aR,11aS,11bR,13aR,13bR)-3a-((((9H-fluoren-9-yl)methoxy)carbonyl)amino)-5a,5b,8,8,11a-pentamethyl-1-(1-oxopropan-2-yl)-2,3,3a,4,5,5a,5b,6,7,7a,8,11,11a,11b,12,13,13a,13b-octadecahydro-1H-cyclopenta[a]chrysen-9-yl)-2-fluorobenzoate

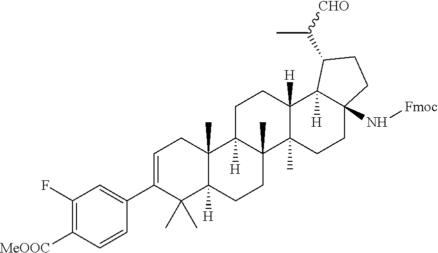

To a solution of methyl 4-((1R,3aS,5aR,5bR,7aR,11aS,11bR,13aR,13bS)-3a-((((9H-fluoren-9-yl)methoxy)carbonyl)amino)-5a,5b,8,8,11a-pentamethyl-1-(2-methyloxiran-2-yl)-2,3,3a,4,5,5a,5b,6,7,7a,8,11,11a,11b,12,13,13a,13b-octadecahydro-1H-cyclopenta[a]chrysen-9-yl)-2-fluorobenzoate (780 mg, 0.975 mmol) in THF (30 mL) at room temperature was added BF₃.OEt₂ (0.247 mL, 1.950 mmol). The reaction mixture was stirred for 2 hours at room temperature, quenched with distilled water (40 mL) and extracted with dichloromethane (2×30 mL). The combined organic phases were dried over sodium sulfate, filtered and concentrated under reduced pressure. The crude was purified using silica gel with 0-35% ethyl acetate/hexanes as the mobile phase to provide the title compound as a mixture of diastereoisomers (white solid, 480 mg, 62%). LCMS: m/e 800.6 (M+H)⁺, 3.40/3.61 min (method 6).

### Step 4. Preparation of methyl 4-((1R,3aS,5aR,5bR,7aR,11aS,11bR,13aR,13bR)-3a-((((9H-fluoren-9-yl)methoxy)carbonyl)amino)-1-(1-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoate

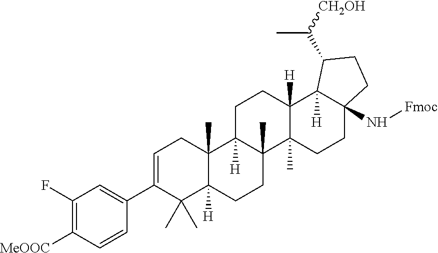

To a solution of methyl 4-((1R,3aS,5aR,5bR,7aR,11aS,11bR,13aR,13bR)-3a-((((9H-fluoren-9-yl)methoxy)carbonyl)amino)-5a,5b,8,8,11a-pentamethyl-1-(1-oxopropan-2-yl)-2,3,3a,4,5,5a,5b,6,7,7a,8,11,11a,11b,12,13,13a,13b-octadecahydro-1H-cyclopenta[a]chrysen-9-yl)-2-fluorobenzoate (60 mg, 0.075 mmol) in THF (2 mL) at room temperature was added sodium borohydride (5.67 mg, 0.150 mmol). The reaction mixture was stirred for 2 hours at room temperature, then quenched with distilled water (2 mL) and extracted with dichloromethane (2×2 mL). The combined organic phases were dried over sodium sulfate, filtered and concentrated under reduced pressure to provide the title compound as a mixture of diasteromers (white solid, 50 mg, 83%). LCMS: m/e 802.6/802.6 (M+H)⁺, 2.98/3.41 min (method 6).

### Step 5. Preparation of methyl 4-((1R,3aS,5aR,5bR,7aR,11aS,11bR,13aR,13bR)-3a-amino-1-(1-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoate

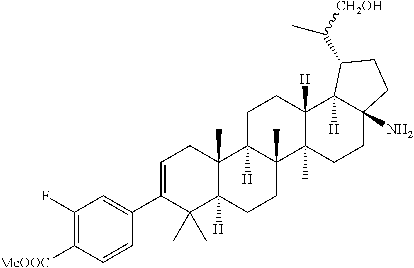

To a solution of methyl 4-((1R,3aS,5aR,5bR,7aR,11aS,11bR,13aR,13bR)-3a-((((9H-fluoren-9-yl)methoxy)carbonyl)amino)-1-(1-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoate (50 mg, 0.062 mmol) in THF (1 mL) at 20° C. was added piperidine (106 mg, 1.247 mmol). The reaction mixture was stirred for 2 hours at 20° C. and then concentrated under reduced pressure. The residue was dissolved in acetonitrile (2 mL) and purified by prep HPLC to provide two diasteromers of the title compound: diasteromer 1 (12 mg, 33%) and diasteromer 2 (20 mg, 55%) as white solids.

LCMS: m/e 580.5/580.6(M+H)⁺, 1.56/1.60 min (method 6).

### Step 6. Preparation of methyl 4-((1R,3aS,5aR,5bR,7aR,11aS,11bR,13aR,13bR)-3a-((2-(1,1-dioxidothiomorpholino)ethyl)amino)-1-(1-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoate

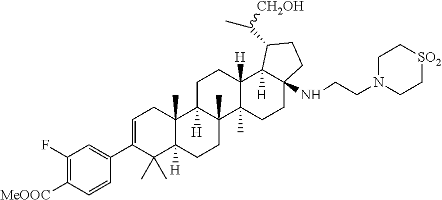

A mixture of methyl 4-((1R,3aS,5aR,5bR,7aR,11aS,11bR,13aR,13bR)-3a-amino-1-(1-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoate (diasteromer 1),4-(2-chloroethyl)thiomorpholine 1,1-dioxide (20.46 mg, 0.103 mmol), potassium phosphate (21.97 mg, 0.103 mmol) and potassium iodide (5.73 mg, 0.034 mmol) in acetonitrile (1 mL) was heated at 120° C. for 15 hours. The reaction mixture was quenched with water (2 mL) and extracted with dichloromethane (2×2 mL). The combined organic phases were dried over sodium sulfate, filtered and concentrated under reduced pressure to provide methyl 4-((1R,3aS,5aR,5bR,7aR,11aS,11bR,13aR,13bR)-3a-((2-(1,1-dioxidothiomorpholino)ethyl)amino)-1-(1-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoate (diasteromer 1) as a white solid (9 mg, 35%). LCMS: m/e 741.44 (M+H)⁺, 2.00 min (method 4). Methyl 4-((1R,3aS,5aR,5bR,7aR,11aS,11bR,13aR,13bR)-3a-((2-(1,1-dioxidothiomorpholino)ethyl)amino)-1-(1-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoate (diasteromer 2) was prepared following the same method described above for diasteromer 1 using methyl 4-((1R,3aS,5aR,5bR,7aR,11aS,11bR,13aR,13bR)-3a-amino-1-(1-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoate (diasteromer 2) as the starting material. The product was isolated as a white solid (22 mg, 86%). LCMS: m/e 741.4(M+H)⁺, 2.24 min (method 4).

Step 7. A mixture of methyl 4-((1R,3aS,5aR,5bR,7aR,11aS,11bR,13aR,13bR)-3a-((2-(1,1-dioxidothiomorpholino)ethyl)amino)-1-(1-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoate (diasteromer 1) (9 mg, 0.012 mmol) and sodium hydroxide (0.297 mL, 0.297 mmol) in acetonitrile (1 mL) was heated at 80° C. for 2 hours. The reaction mixture was filtered and purified by prep HPLC to provide Example A 4: 4-((1R,3aS,5aR,5bR,7aR,11aS,11bR,13aR,13bR)-3a-((2-(1,1-dioxidothiomorpholino)ethyl)amino)-1-(1-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoic acid (diasteromer 1) as a white solid (5 mg, 22%). LCMS: m/e 727.6 (M+H)⁺, 1.34 min (method 6). ¹H NMR (500 MHz, Acetic) δ 7.97 (t, J=8.0 Hz, 1H), 7.10 (dd, J=8.0, 1.4 Hz, 1H), 7.04 (dd, J=11.8, 1.3 Hz, 1H), 5.44 (d, J=4.7 Hz, 1H), 3.86 (dd, J=11.2, 5.7 Hz, 1H), 3.67-3.54 (m, 1H), 3.47-3.24 (m, 8H), 3.22-3.00 (m, 4H), 2.63-2.43 (m, 1H), 2.35-2.22 (m, 2H), 2.17-1.30 (m, 21H), 1.31 (s, 3H), 1.13 (s, 3H), 1.12 (s, 3H), 1.05 (s, 3H), 1.02 (s, 3H), 1.00 (d, J=6.9 Hz, 3H).

### Example A5

4-((1R,3aS,5aR,5bR,7aR,11aS,11bR,13aR,13bR)-3a-((2-(1,1-dioxidothiomorpholino)ethyl)amino)-1-(1-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoic acid (diasteromer 2) was prepared following the method described above for diasteromer 1 using methyl 4-((1R,3aS,5aR,5bR,7aR,11aS,11bR,13aR,13bR)-3a-((2-(1,1-dioxidothiomorpholino)ethyl)amino)-1-(1-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoate (diasteromer 2) as the starting material. The product was isolated as a white solid (13 mg, 57%). LCMS: m/e 727.6(M+H)⁺, 1.93 min (method 6).¹H NMR (400 MHz, ACETONITRILE-d₃) δ 7.81 (t, J=7.9 Hz, 1H), 7.03 (dd, J=8.0, 1.5Hz, 1H), 7.00-6.93 (m, 1H), 5.31 (dd, J=6.0, 1.8 Hz, 1H), 3.34-2.89 (m, 14H), 2.65-2.47 (m, 1H), 2.18-1.05 (m, 23H), 1.16 (s, 3H), 1.04 (s, 3H), 0.98 (s, 3H), 0.95 (s, 3H), 0.93 (s, 3H), 0.76 (d, J=6.8 Hz, 3H).

### Example A6 and Example A7

### Preparation of 4-((1R,3aS,5aR,5bR,7aR,11aS,11bR,13aR,13bR)-1-(1-carboxyethyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoic acid (diasteromer 1 and diasteromer 2)

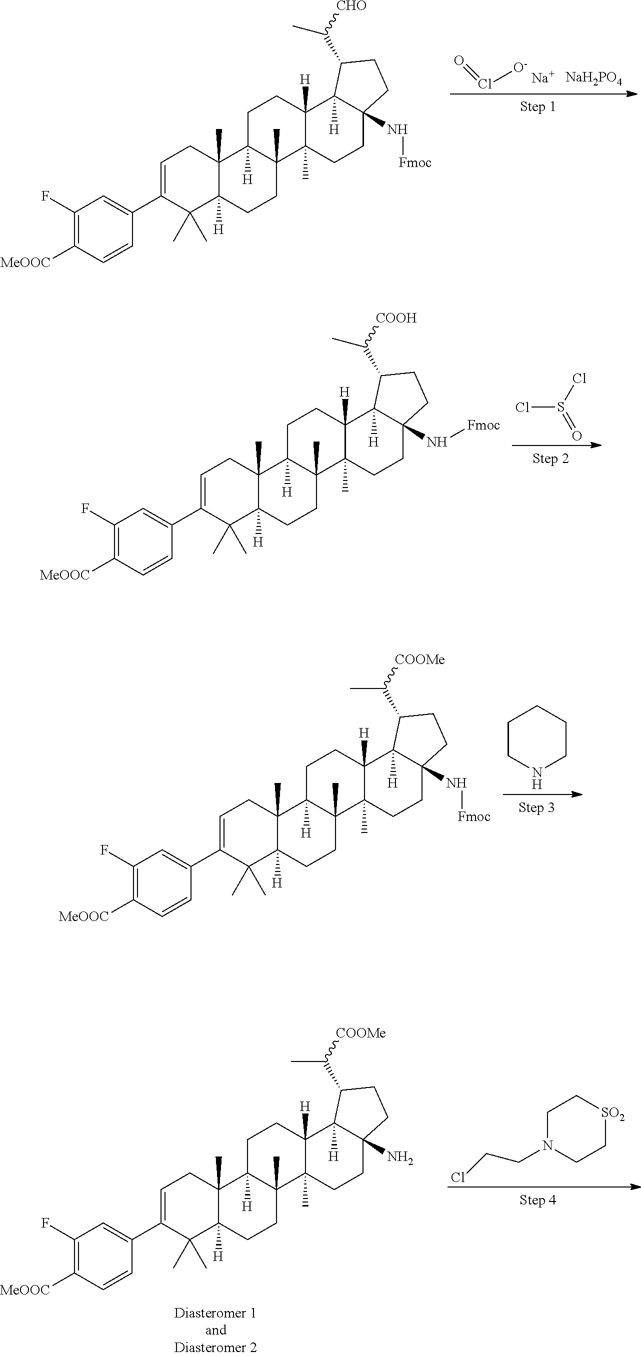

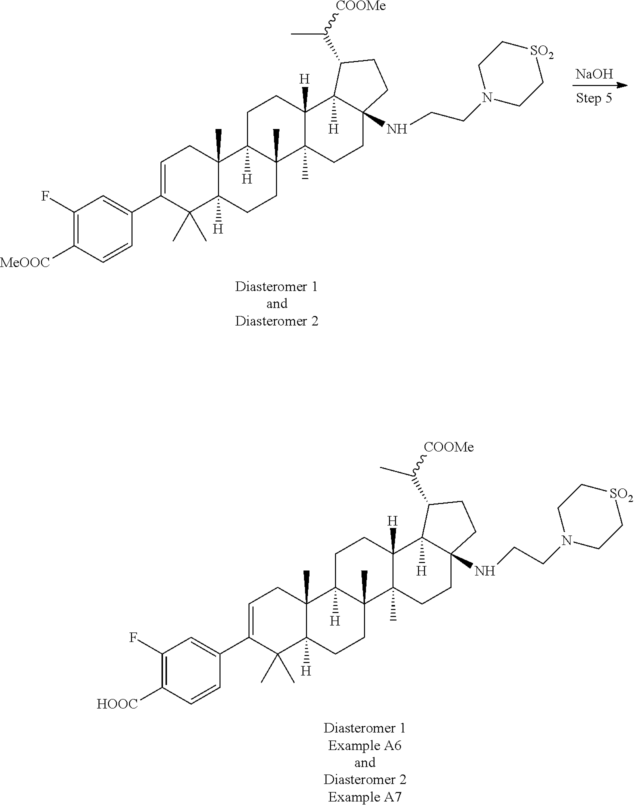

### Step 1. Preparation of 2-((1R,3aS,5aR,5bR,7aR,11aS,11bR,13aR,13bR)-3a-((((9H-fluoren-9-yl)methoxy)carbonyl)amino)-9-(3-fluoro-4-(methoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-1-yl)propanoic acid

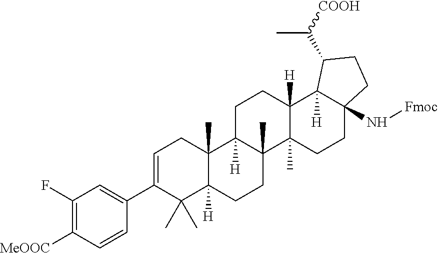

To a solution of methyl 4-((1R,3aS,5aR,5bR,7aR,11aS,11bR,13aR,13bR)-3a-((((9H-fluoren-9-yl)methoxy)carbonyl)amino)-5a,5b,8,8,11a-pentamethyl-1-(1-oxopropan-2-yl)-2,3,3a,4,5,5a,5b,6,7,7a,8,11,11a,11b,12,13,13a,13b-octadecahydro-1H-cyclopenta[a]chrysen-9-yl)-2-fluorobenzoate (diasteromeric mixture) (150 mg, 0.187 mmol) in tBuOH (4 mL) and THF (8 mL) at 20° C. was added a solution of sodium dihydrogenphosphate (202 mg, 1.687 mmol) and sodium chlorite (115 mg, 1.275 mmol) in water (5 mL) over 0.5-1 h. The reaction mixture was stirred for 1 additional hour at 20° C., quenched with water (10 mL) and extracted with dichloromethane (2×10 mL). The combined organic phases were dried over sodium sulfate, filtered and concentrated under reduced pressure to provide the title compound as a mixture of diasteromers (white solid, 100 mg, 65%). LCMS: m/e 816.3 (M+H)⁺, 2.68 min (method 4).

### Step 2. Preparation of methyl 4-((1R,3aS,5aR,5bR,7aR,11aS,11bR,13aR,13bR)-3a-((((9H-fluoren-9-yl)methoxy)carbonyl)amino)-1-(1-methoxy-1-oxopropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoate

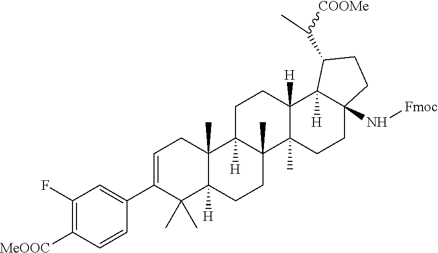

To a solution of 2-((1R,3aS,5aR,5bR,7aR,11aS,11bR,13aR,13bR)-3a-((((9H-fluoren-9-yl)methoxy)carbonyl)amino)-9-(3-fluoro-4-(methoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-1-yl)propanoic acid (100 mg, 0.123 mmol) in dichloromethane (2 mL) at 20° C. was added thionyl chloride (0.045 mL, 0.613 mmol). The reaction mixture was stirred for 2 hours at 20° C. and then concentrated under reduced pressure to provide the brown residue. Methanol (4 mL) was slowly added to the residue and the mixture was stirred for 10 additional minutes. The reaction mixture was then concentrated under reduced pressure to provide the title compound as a mixture of diasteromers (brown solid, 96 mg, 94%). LCMS: m/e 830.35/830.34 (M+H)⁺, 3.59/3.83 min (method 4).

### Step 3. Preparation of methyl 4-((1R,3aS,5aR,5bR,7aR,11aS,11bR,13aR,13bR)-3a-amino-1-(1-methoxy-1-oxopropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoate (diasteromer 1 and diasteromer 2)

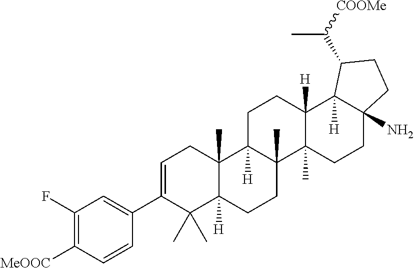

To a solution of methyl 4-((1R,3aS,5aR,5bR,7aR,11aS,11bR,13aR,13bR)-3a-((((9H-fluoren-9-yl)methoxy)carbonyl)amino)-1-(1-methoxy-1-oxopropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoate (96 mg, 0.116 mmol) in THF (1 mL) at 20° C. was added piperidine (197 mg, 2.313 mmol). The reaction mixture was stirred for 1 hour and then concentrated under reduced pressure. The residue was dissolved in acetonitrile (1 mL) and the clear solution was purified by prep HPLC to provide two diasteromers of the title compound: methyl 4-((1R,3aS,5aR,5bR,7aR,11aS,11bR,13aR,13bR)-3a-amino-1-(1-methoxy-1-oxopropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoate (diasteromer 1) as a white solid (12 mg, 17%). LCMS: m/e 607.24 (M)⁺/591.25(M-NH₂)⁺, 1.88 min (method 4). And methyl 4-((1R,3aS,5aR,5bR,7aR,11aS,11bR,13aR,13bR)-3a-amino-1-(1-methoxy-1-oxopropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoate (diasteromer 2) as a white solid (23 mg, 33%). LCMS: m/e 607.24 (M)⁺/591.25(M-NH₂)⁺, 2.00 min (method 4).

### Step 4. Preparation of methyl 4-((1R,3aS,5aR,5bR,7aR,11aS,11bR,13aR,13bR)-3a-((2-(1,1-dioxidothiomorpholino)ethyl)amino)-1-(1-methoxy-1-oxopropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoate (diasteromer 1 and diasteromer 2)

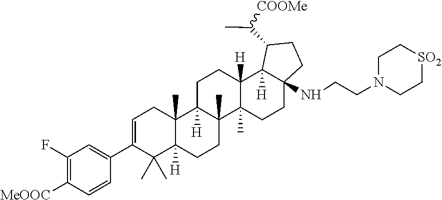

A mixture of methyl 4-((1R,3aS,5aR,5bR,7aR,11aS,11bR,13aR,13bR)-3a-amino-1-(1-methoxy-1-oxopropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoate (diasteromer 1) (12 mg, 0.020 mmol), potassium phosphate (24.10 mg, 0.114 mmol), 4-(2-chloroethyl)thiomorpholine 1,1-dioxide (22.44 mg, 0.114 mmol) and potassium iodide (6.28 mg, 0.038 mmol) in acetonitrile (1 mL) were heated at 120° C. for 15 hours. The reaction mixture was quenched with water (2 mL), extracted with dichloromethane (2×2 mL), dried over sodium sulfate, filtered and concentrated under reduced pressure to provide methyl 4-((1R,3aS,5aR,5bR,7aR,11aS,11bR,13aR,13bR)-3a-((2-(1,1-dioxidothiomorpholino)ethyl)amino)-1-(1-methoxy-1-oxopropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoate (diasteromer 1) as a white solid (10 mg, 34%). LCMS: m/e 769.33 (M+H)⁺, 2.24 min (method 4).

Methyl 4-((1R,3aS,5aR,5bR,7aR,11aS,11bR,13aR,13bR)-3a-((2-(1,1-dioxidothiomorpholino)ethyl)amino)-1-(1-methoxy-1-oxopropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoate (diasteromer 2) was prepared following the method described above for the synthesis of diasteromer 1 using methyl methyl 4-((1R,3aS,5aR,5bR,7aR,11aS,11bR,13aR,13bR)-3a-amino-1-(1-methoxy-1-oxopropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoate (diasteromer 2) as the starting material. The product was isolated as a white solid (16 mg, 55%). LCMS: m/e 769.33(M+H)⁺, 2.26 min (method 4).

Step 5. A mixture of methyl 4-((1R,3aS,5aR,5bR,7aR,11aS,11bR,13aR,13bR)-3a-((2-(1,1-dioxidothiomorpholino)ethyl)amino)-1-(1-methoxy-1-oxopropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoate (diasteromer 1) (10 mg, 0.013 mmol) and sodium hydroxide (0.130 mL, 0.130 mmol) was heated up at 80° C. for 2 hours. The reaction mixture was filtered and purified by prep HPLC to provide 4-((1R,3aS,5aR,5bR,7aR,11aS,11bR,13aR,13bR)-1-(1-carboxyethyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoic acid (diasteromer 1) as a white solid (4 mg, 39%). LCMS: m/e 741.25 (M+H)⁺, 1.55 min (method 4). ¹H NMR (500 MHz, ACETONITRILE-d₃) δ 7.85 (t, J=7.9 Hz, 1H), 7.08 (dd, J=8.0, 1.6 Hz, 1H), 7.03 (dd, J=12.1, 1.3 Hz, 1H), 5.37 (dd, J=6.2, 1.8 Hz, 1H), 3.34-3.07 (m, 7H), 3.06-2.88 (m, 5H), 2.81-2.66 (m, 1H), 2.36-2.14 (m, 2H), 2.07-1.26 (m, 21H), 1.22 (s, 3H), 1.15 (d, J=7.1 Hz, 3H), 1.06 (s, 3H), 1.05 (s, 3H), 1.00 (s, 3H), 0.98 (s, 3H).

Example A7: 4-((1R,3aS,5aR,5bR,7aR,11aS,11bR,13aR,13bR)-1-(1-carboxyethyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoic acid (diasteromer 2) was prepared following the method described above for the synthesis of 4-((1R,3aS,5aR,5bR,7aR,11aS,11bR,13aR,13bR)-1-(1-carboxyethyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoic acid (diasteromer 1) using methyl 4-((1R,3aS,5aR,5bR,7aR,11aS,11bR,13aR,13bR)-3a-((2-(1,1-dioxidothiomorpholino)ethyl)amino)-1-(1-methoxy-1-oxopropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)-2-fluorobenzoate (diasteromer 2) as starting material. The title compound was isolated as a white solid (7 mg, 69⁰%). LCMS: m/e 741.5(M+H)⁺, 2.21 min (method 4).¹H NMR (500 MHz, ACETONITRILE-d₃) δ 7.85 (t, J=8.0 Hz, 1H), 7.08 (dd, J=8.0, 1.5Hz, 1H), 7.03 (dd, J=12.1, 1.3 Hz, 1H), 5.37 (dd, J=6.1, 1.7 Hz, 1H), 3.31-3.08 (m, 7H), 3.07-2.97 (m, 4H), 2.95-2.85 (m, 1H), 2.84-2.70 (m, 2H), 2.18 (dd, J=17.3, 6.4 Hz, 1H), 2.09-1.27 (m, 21H), 1.21 (s, 3H), 1.09 (s, 3H), 1.08 (d, J=5.4 Hz, 3H), 1.15 (s, 3H), 1.00 (s, 3H), 0.98 (s, 3H).

### Example A8

### Preparation of (1R,3aR,5aR,5bR,7aR,11aS,11bR,13aR,13bR)-1-((tert-butoxycarbonyl)amino)-9-(4-carboxycyclohex-1-en-1-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid

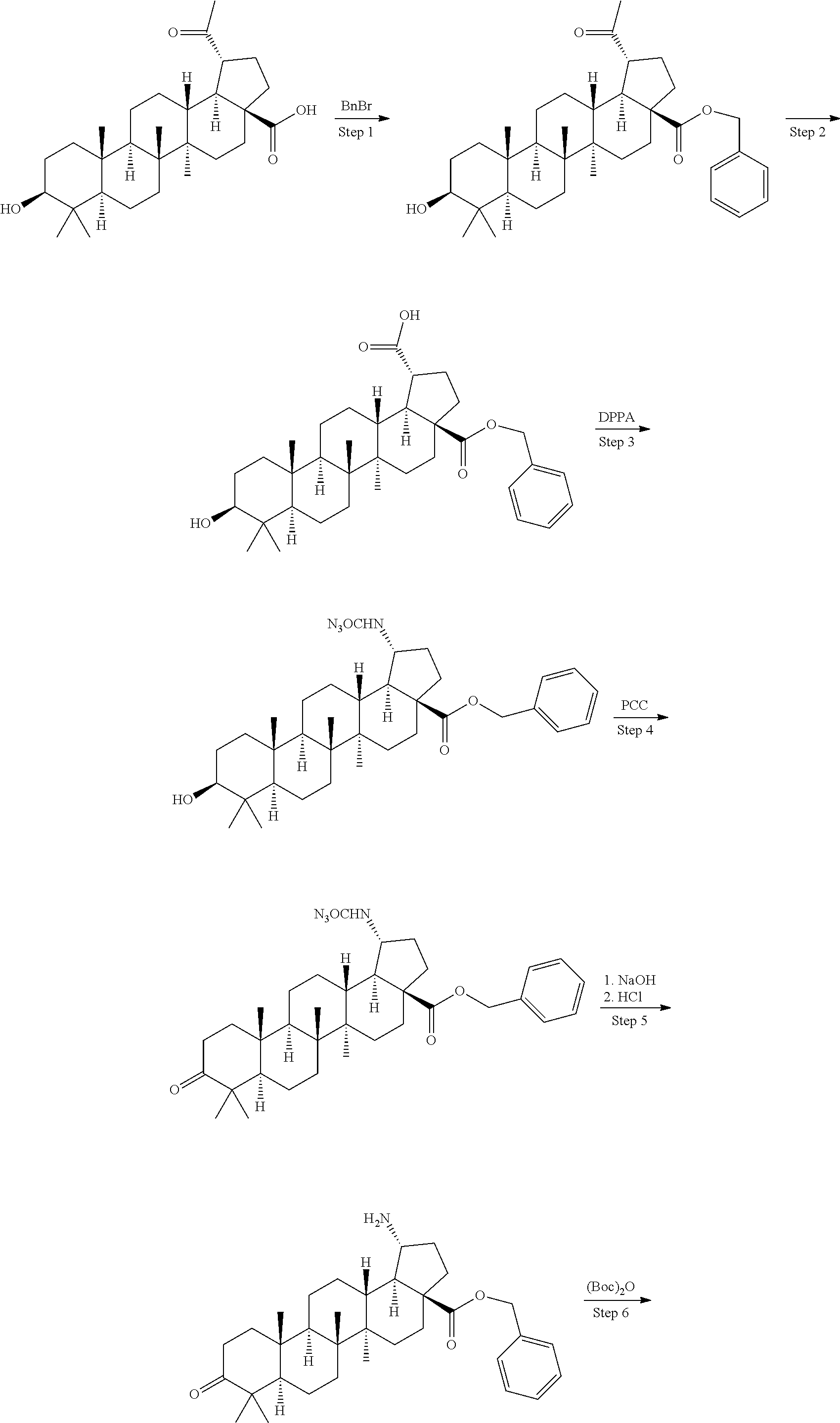

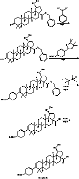

### Step 1. Preparation of (1R,3aS,5aR,5bR,7aR,9S,11aR,11bR,13aR,13bS)-benzyl 1-acetyl-9-hydroxy-5a,5b,8,8,11a-pentamethylicosahydro-1H-cyclopenta[a]chrysene-3a-carboxylate

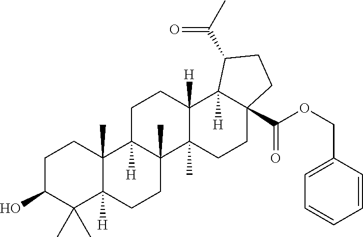

A mixture of (1R,3aS,5aR,5bR,7aR,9S,11aR,11bR,13aR,13bS)-1-acetyl-9-hydroxy-5a,5b,8,8,11a-pentamethylicosahydro-1H-cyclopenta[a]chrysene-3a-carboxylic acid (1.6 g, 3.49 mmol), potassium carbonate (0.964 g, 6.98 mmol) and (bromomethyl)benzene (0.435 mL, 3.66 mmol) in DMF (10 mL) was heated at 60° C. for 18 hours. The reaction mixture was cooled down to room temperature and 100 mL water was added. A white precipitate was collected and dried under vacuum to provide the desired product as a white solid (1.7 g, 89%). LCMS: m/e 549.3 (M+H)⁺, 2.54 min (method 4).

### Step 2. Preparation of (1R,3aS,5aR,5bR,7aR,9S,11aR,11bR,13aR,13bS)-3a-((benzyloxy)carbonyl)-9-hydroxy-5a,5b,8,8,11a-pentamethylicosahydro-1H-cyclopenta[a]chrysene-1-carboxylic acid

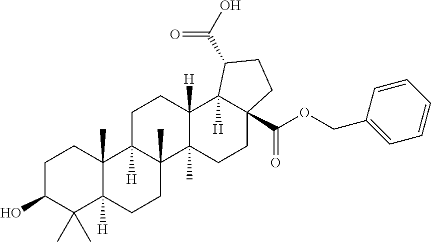

To a solution of sodium hydroxide (1.749 g, 43.7 mmol) in water (30 mL) was slowly added dibromine (0.789 mL, 15.31 mmol) at 0° C., the reaction mixture was stirred for 20 min at 0° C., this fresh made orange solution was slowly added to a solution of (1R,3aS,5aR,5bR,7aR,9S,11aR,11bR,13aR,13bS)-benzyl 1-acetyl-9-hydroxy-5a,5b,8,8,11a-pentamethylicosahydro-1H-cyclopenta[a]chrysene-3a-carboxylate (1.2 g, 2.187 mmol) in dioxane (40 mL). The reaction mixture was stirred at 0° C. for 4 hours and then warmed up to rt and stirred for 15 hours. The reaction mixture was then neutralized by concentrated HCl to pH=3-4 and extracted with dichloromethane (2×40 mL). The combined organic phases were dried over sodium sulfate, filtered and concentrated under reduced pressure. The crude was purified using silica gel with 0-60% ethyl acetate/hexanes as the mobile phase to provide the desired product as a white solid (1.1 g, 91%). LCMS: m/e 549.18 (M−H)⁻, 2.26 min (method 4).

### Step 3. Preparation of (1R,3aR,5aR,5bR,7aR,9S,11aR,11bR,13aR,13bR)-benzyl 1-((azidocarbonyl)amino)-9-hydroxy-5a,5b,8,8,11a-pentamethylicosahydro-1H-cyclopenta[a]chrysene-3a-carboxylate

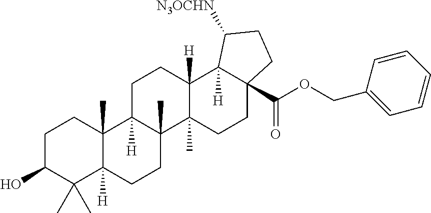

A mixture of (1R,3aS,5aR,5bR,7aR,9S,11aR,11bR,13aR,13bS)-3a-((benzyloxy)carbonyl)-9-hydroxy-5a,5b,8,8,11a-pentamethylicosahydro-1H-cyclopenta[a]chrysene-1-carboxylic acid (1100 mg, 1.997 mmol), diphenyl phosphorazidate (1.076 mL, 4.99 mmol) and triethylamine (0.835 mL, 5.99 mmol) in dioxane (30 mL) was heated at 75° C. for 2 hours until TLC indicated the sm was consumed. The reaction mixture was concentrated under reduced pressure and the residue was purified using silica gel with 0-42% ethyl acetate/hexanes as the mobile phase to provide the title compound as a white solid (1.2 g, 100%). LCMS: m/e 589.29 (M−H)⁻, 2.40 min (method 4).

### Step 4. Preparation of (1R,3aR,5aR,5bR,7aR,11aR,11bR,13aR,13bR)-benzyl 1-((azidocarbonyl)amino)-5a,5b,8,8,11a-pentamethyl-9-oxoicosahydro-1H-cyclopenta[a]chrysene-3a-carboxylate

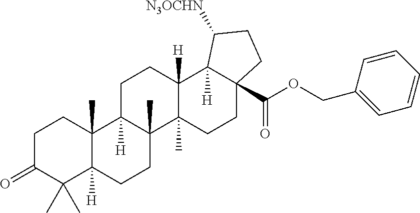

A mixture of (1R,3aR,5aR,5bR,7aR,9S,11aR,11bR,13aR,13bR)-benzyl 1-((azidocarbonyl)amino)-9-hydroxy-5a,5b,8,8,11a-pentamethylicosahydro-1H-cyclopenta[a]chrysene-3a-carboxylate (1.2 g, 2.031 mmol) and PCC (1.095 g, 5.08 mmol) in THF (20 mL) was stirred at 20° C. for 17 hours. The reaction mixture was concentrated under reduced pressure and the residue was purified on silica gel with 0-35% ethyl acetate/hexanes as the mobile phase to provide the product as a colorless oil. (1.15 g, 96%). LCMS: m/e 589.29 (M+H)⁺, 2.48 min (method 4).

### Step 5. Preparation of (1R,3aR,5aR,5bR,7aR,11aR,11bR,13aR,13bR)-benzyl 1-amino-5a,5b,8,8,11a-pentamethyl-9-oxoicosahydro-1H-cyclopenta[a]chrysene-3a-carboxylate

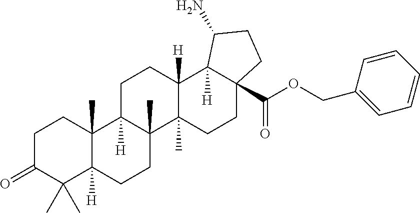

To a solution of (1R,3aR,5aR,5bR,7aR,11aR,11bR,13aR,13bR)-benzyl 1-((azidocarbonyl)amino)-5a,5b,8,8,11a-pentamethyl-9-oxoicosahydro-1H-cyclopenta[a]chrysene-3a-carboxylate (1.15 g, 1.953 mmol) in THF (20 mL) was added 1N sodium hydroxide (9.77 mL, 9.77 mmol). The reaction mixture was stirred for 1 hour until the sm was consumed. To the reaction mixture was added HCl (0.593 mL, 19.53 mmol), the reaction mixture was stirred for another 2 hours and extracted with dichloromethane (3×15 mL), the combined organic phases were dried over sodium sulfate, filtered and concentrated under reduced pressure to provide the title compound as a pale yellow solid. (1.0 g, 96%). LCMS: m/e 520.28 (M+H)⁺, 2.30 min (method 4).

### Step 6. Preparation of (1R,3aR,5aR,5bR,7aR,11aR,11bR,13aR,13bR)-benzyl 1-((tert-butoxycarbonyl)amino)-5a,5b,8,8,11a-pentamethyl-9-oxoicosahydro-1H-cyclopenta[a]chrysene-3a-carboxylate

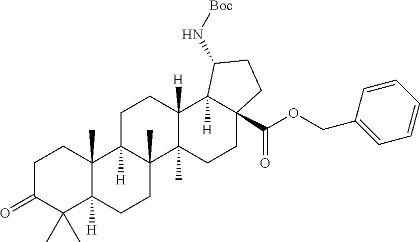

A mixture of (1R,3aR,5aR,5bR,7aR,11aR,11bR,13aR,13bR)-benzyl 1-amino-5a,5b,8,8,11a-pentamethyl-9-oxoicosahydro-1H-cyclopenta[a]chrysene-3a-carboxylate(97055-013, 011) (1.05 g, 2.020 mmol), di-tert-butyl dicarbonate (0.704 mL, 3.03 mmol) and triethylamine (0.845 mL, 6.06 mmol) in THF (10 mL) was stirred at 20° C. for 48 hours. The reaction mixture was quenched with distilled water (15 mL) and extracted with dichloromethane (2×15 mL), the organic layers were combined, dried over sodium sulfate, filtered and concentrated under reduced pressure to provide the crude, the crude was purified using silica gel with 0-40% ethyl acetate/hexanes as the mobile phase to provide the product as a white solid (0.83 g, 66%). ¹H NMR (500 MHz, CHLOROFORM-d) δ 7.47-7.31 (m, 5H), 5.14 (d, J=6.5Hz, 2H), 2.63-2.47 (m, 1H), 2.46-2.36 (m, 1H), 2.29 (d, J=12.0 Hz, 1H), 2.23-2.12 (m, 2H), 2.01-1.90 (m, 1H), 1.86 (dd, J=12.5, 7.9 Hz, 1H), 1.75-1.64 (m, 1H), 1.62-1.09 (m, 17H), 1.46 (s, 9H), 1.09 (s, 3H), 1.04 (s, 3H), 0.94 (s, 3H), 0.93 (s, 3H), 0.75 (s, 3H).

### Step 7. Preparation of (1R,3aR,5aR,5bR,7aR,11aR,11bR,13aR,13bR)-benzyl 1-((tert-butoxycarbonyl)amino)-5a,5b,8,8,11a-pentamethyl-9-(((trifluoromethyl) sulfonypoxy)-2,3,3a,4,5,5a,5b,6,7,7a,8,11,11a,11b,12,13,13a,13b-octadecahydro-1H-cyclopenta[a]chrysene-3a-carboxylate

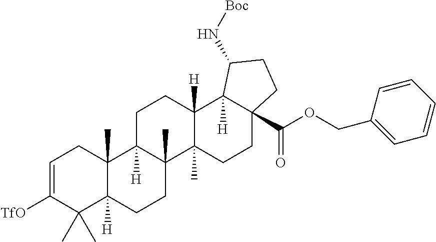

To a solution of (1R,3aR,5aR,5bR,7aR,11aR,11bR,13aR,13bR)-benzyl 1-((tert-butoxycarbonyl)amino)-5a,5b,8,8,11a-pentamethyl-9-oxoicosahydro-1H-cyclopenta[a]chrysene-3a-carboxylate (0.83 g, 1.339 mmol) in THF (10 mL) was added potassium bis(trimethylsilyl)amide (2.94 mL, 2.68 mmol) at −78° C. The reaction mixture was stirred at −78° C. for 15 minutes, 1,1,1-trifluoro-N-phenyl-N-((trifluoromethyl)sulfonyl)methanesulfonamide (0.526 g, 1.473 mmol) in 4 mL THF was added. The reaction mixture was stirred for 2 hours at −78° C. The reaction mixture was quenched with distilled water (20 mL) and extracted with ethyl acetate (2×20 mL). The organic phases were combined, dried over sodium sulfate, and concentrated under reduced pressure. The crude was purified using silica gel with 0-33% ethyl acetate/hexanes as the mobile phase to provide the title compound as a colorless oil, which was dried under vacuum to give a white foam solid (0.5 g, 50%). ¹H NMR (500 MHz, CHLOROFORM-d) δ 7.49-7.30 (m, 5H), 5.58 (dd, J=6.7, 1.8 Hz, 1H), 5.14 (d, J=8.4 Hz, 2H), 2.29 (d, J=12.0 Hz, 1H), 2.24-2.12 (m, 3H), 1.99-1.07 (m, 19H), 1.46 (s, 9H), 1.13 (s, 3H), 1.03 (s, 3H), 0.93 (s, 3H), 0.91 (s, 3H), 0.73 (s, 3H).

### Step 8. Preparation of (1R,3aR,5aR,5bR,7aR,11aS,11bR,13aR,13bR)-benzyl 1-((cert-butoxycarbonyl)amino)-9-(4-(ethoxycarbonyl)cyclohex-1-en-1-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylate

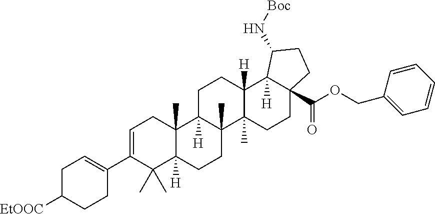

A mixture of ethyl 4-(4,4,5,5-tetramethyl-1,3,2-dioxaborolan-2-yl)cyclohex-3-enecarboxylate (149 mg, 0.532 mmol), (1R,3aR,5aR,5bR,7aR,11aR,11bR,13aR,13bR)-benzyl 1-((tert-butoxycarbonyl)amino)-5a,5b,8,8,11a-pentamethyl-9-(((trifluoromethyl)sulfonyl)oxy)-2,3,3a,4,5,5a,5b,6,7,7a,8,11,11a,11b,12,13,13a,13b-octadecahydro-1H-cyclopenta[a]chrysene-3a-carboxylate (200 mg, 0.266 mmol), tetrakis(triphenylphosphine)palladium(0) (15.37 mg, 0.013 mmol) and sodium bicarbonate (112 mg, 1.330 mmol) in dioxane (5 mL) and water (5 mL) was heated at 76° C. for 3 hours. The reaction mixture was quenched with distilled water (10 mL), and extracted with ethyl acetate (3×10 mL). The extracts were combined, dried over sodium sulfate, filtered and concentrated under reduced pressure. The crude was purified using silica gel with 0-25% ethyl acetate/hexanes as the mobile phase to provide the title compound as a colorless oil. (0.15 g, 75%). LCMS: m/e 778.65 (M+Na)⁺, 3.77 min (method 6).

### Step 9. Preparation of (1R,3aR,5aR,5bR,7aR,11aS,11bR,13aR,13bR)-1-((tert-butoxycarbonyl)amino)-9-(4-(ethoxycarbonyl)cyclohex-1-en-1-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid

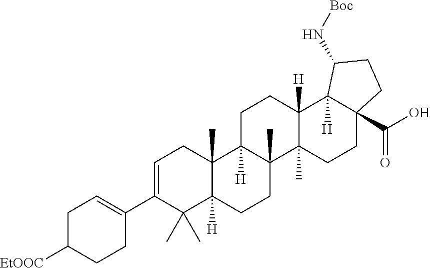

A mixture of (1R,3aR,5aR,5bR,7aR,11aS,11bR,13aR,13bR)-benzyl 1-((tert-butoxycarbonyl)amino)-9-(4-(ethoxycarbonyl)cyclohex-1-en-1-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylate (80 mg, 0.106 mmol), tert-butyldimethylsilane (24.61 mg, 0.212 mmol), palladium (II) acetate (11.88 mg, 0.053 mmol) and triethylamine (0.044 mL, 0.317 mmol) in dichloroethane (2 mL) was heated at 60° C. for 3 hours. Then the mixture was filtered through a pad of silica gel to remove the Pd catalyst, the filtrates were concentrated under reduced pressure to provide the crude as yellow oil. This yellow oil was dissolved in 2 mL THF and treated with TBAF (111 mg, 0.317 mmol). The mixture was stirred for 2 hours until starting material was consumed. The reaction mixture was treated with 1 N HCl until reaching pH 4. Water (2 mL) was added and a white precipitate formed. The precipitate was collected and dried to provide the title compound as a white solid (45 mg, 64%). LCMS: m/e 688.6 (M+Na)⁺, 2.71 min (method 6).

Step 10. A solution of (1R,3aR,5aR,5bR,7aR,11aS,11bR,13aR,13bR)-1-((tert-butoxycarbonyl)amino)-9-(4-(ethoxycarbonyl)cyclohex-1-en-1-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid (6 mg, 9.01 mmol) and sodium hydroxide (0.090 mL, 0.090 mmol) in acetonitrile (1 mL) was heated at 80° C. for 3 hours. The reaction mixture was filtered and purified by HPLC to provide (1R,3aR,5aR,5bR,7aR,11aS,11bR,13aR,13bR)-1-((tert-butoxycarbonyl)amino)-9-(4-carboxycyclohex-1-en-1-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid as a white solid (1.5 mg, 25%). LCMS: m/e 636.23 (M−H)⁻, 2.28 min (method 4).¹H NMR (400 MHz, CHLOROFORM-d) δ 5.39 (br. s., 1H), 5.21 (t, J=5.6 Hz, 1H), 2.80-1.04 (m, 30H), 1.46 (s, 9H), 1.01-0.77 (m, 15H).

### Example A9

### Preparation of 4-((1R,3aR,5aR,5bR,7aR,11aS,11bR,13aR,13bR)-1-((tert-butoxycarbonyl)amino)-3a-((2-(dimethylamino)ethyl)carbamoyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)cyclohex-3-enecarboxylic acid

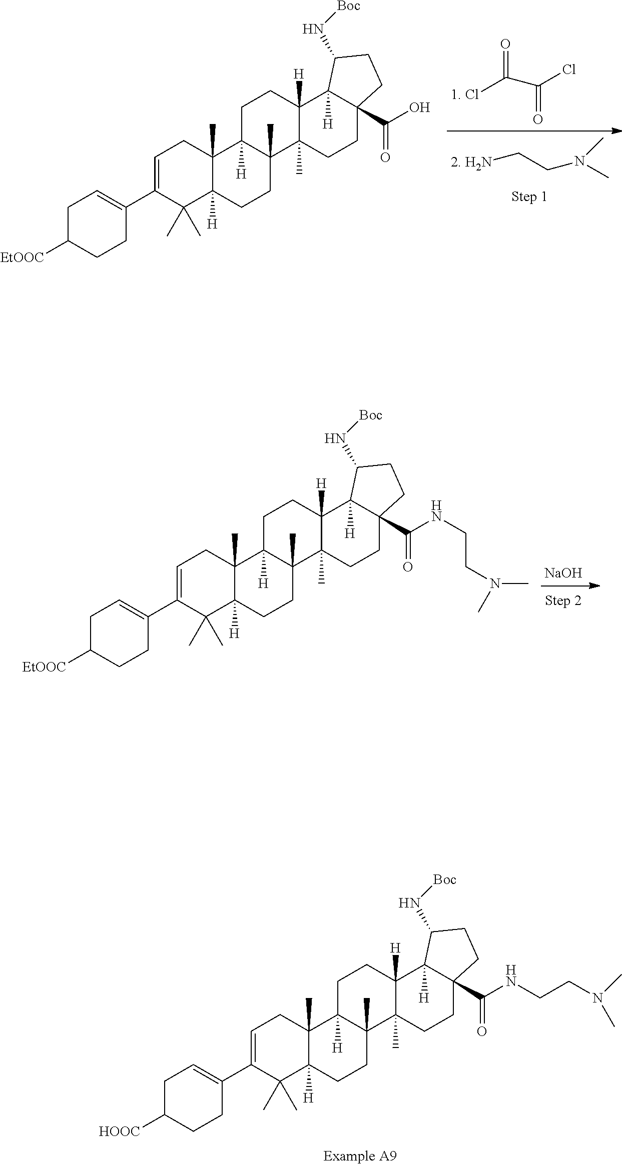

### Step 1. Preparation of ethyl 4-((1R,3aR,5aR,5bR,7aR,11aS,11bR,13aR,13bR)-1-((tert-butoxycarbonyl)amino)-3a-((2-(dimethylamino)ethyl)carbamoyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)cyclohex-3-enecarboxylate

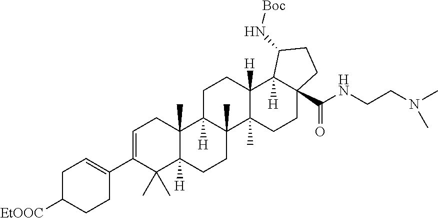

To a solution of (1R,3aR,5aR,5bR,7aR,11aS,11bR,13aR,13bR)-1-((tert-butoxycarbonyl)amino)-9-(4-(ethoxycarbonyl)cyclohex-1-en-1-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylic acid (10 mg, 0.015 mmol) in dichloromethane (1 mL) was added oxalyl dichloride (0.015 mL, 0.030 mmol) at room temperature, the reaction mixture was stirred for 1 hour until starting material was consumed. The reaction mixture was concentrated under reduced pressure and dried under vacuum for 2 hours to provide the intermediate acid chloride as yellow oil. To a solution of N1,N1-dimethylethane-1,2-diamine (1.986 mg, 0.023 mmol) and Hunig's Base (5.25 μL, 0.030 mmol) in dichloromethane (1 mL) was added acid chloride made previously in dichloromethane (1 mL), the reaction mixture was stirred for 16 hours, quenched with distilled water (2 mL) and extracted with dichloromethane (2×2 mL), the combined organic phases were washed with brine (3 mL), dried over sodium sulfate, filtered and concentrated under reduced pressure to provide the title compound as a brown oil. (6 mg, 54%). LCMS: m/e 736.6 (M+H)⁺, 3.02 min (method 3).

### Step 2. Preparation of 4-((1R,3aR,5aR,5bR,7aR,11aS,11bR,13aR,13bR)-1-((tert-butoxycarbonyl)amino)-3a-((2-(dimethylamino)ethyl)carbamoyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)cyclohex-3-enecarboxylic acid

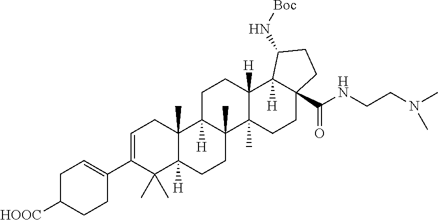

A mixture of ethyl 4-((1R,3aR,5aR,5bR,7aR,11aS,11bR,13aR,13bR)-1-((tert-butoxycarbonyl)amino)-3a-((2-(dimethylamino)ethyl)carbamoyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)cyclohex-3-enecarboxylate (6 mg, 8.15 mmol) and NaOH (0.082 mL, 0.082 mmol) in dioxane (1 mL) was heated up at 80° C. for 2 hours. The reaction mixture was filtered and purified by HPLC to provide the desired product as a colorless oil (1.1 mg, 18%). LCMS: m/e 708.5 (M+H)⁺, 1.76 min (method 6). ¹H NMR (400 MHz, CHLOROFORM-d) δ 7.51 (s, 1H), 5.48-5.31 (m, 1H), 5.20 (d, J=6.3 Hz, 1H), 3.84-3.46 (m, 2H), 3.19 (d, J=5.5Hz, 2H), 2.86 (br. s., 6H), 2.60 (br. s., 1H), 2.42-1.04 (m, 29H), 1.44 (s, 9H), 1.01-0.76 (m, 15H).

### Example B1

### Preparation of 4-((1R,3aS,5aR,5bR,7aR,11aS,11bR,13aR,13bS)-3a-((2-(1,1-dioxidothiomorpholino)ethyl)amino)-1-(2-hydroxy-1-methoxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid.

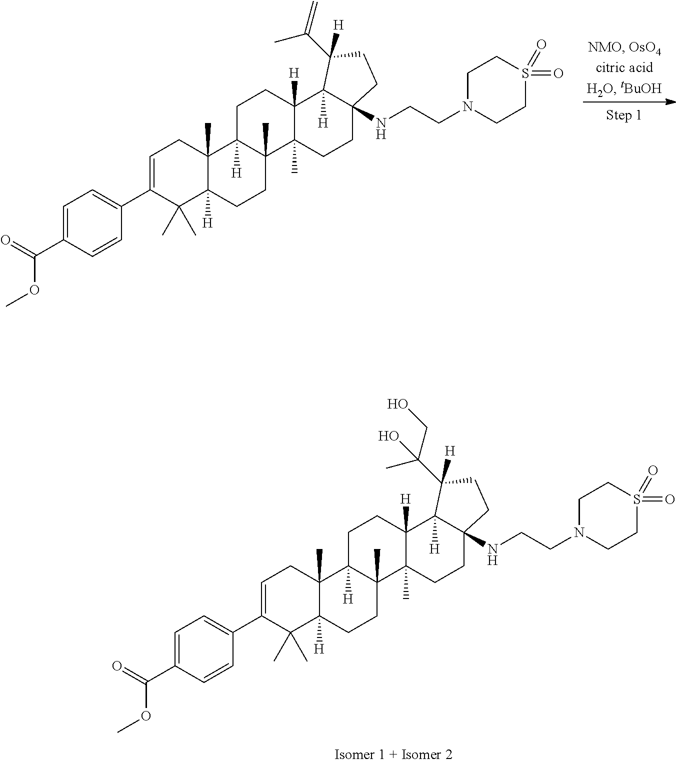

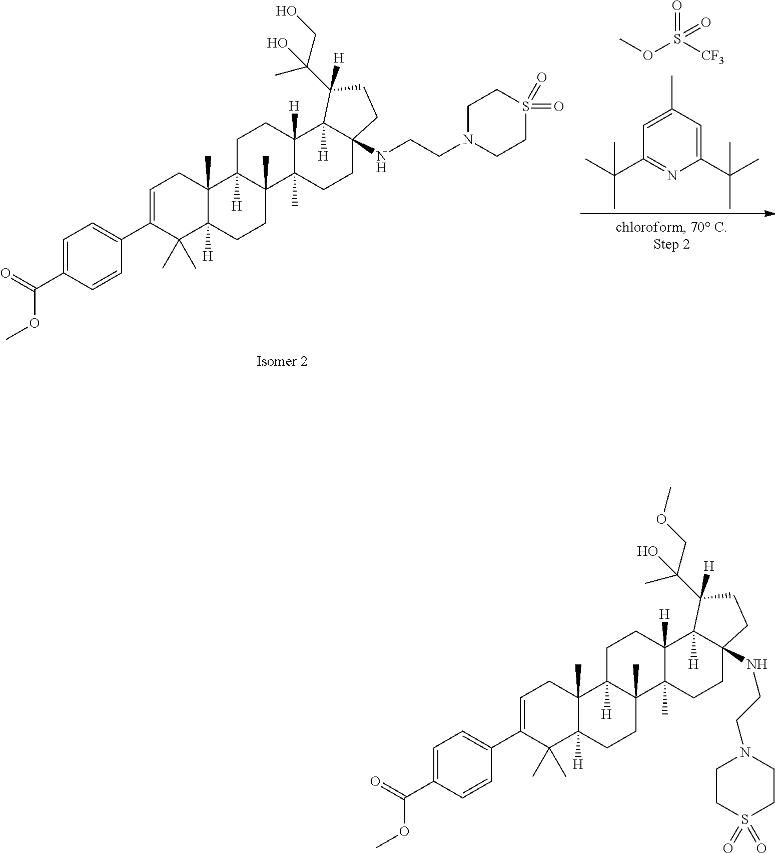

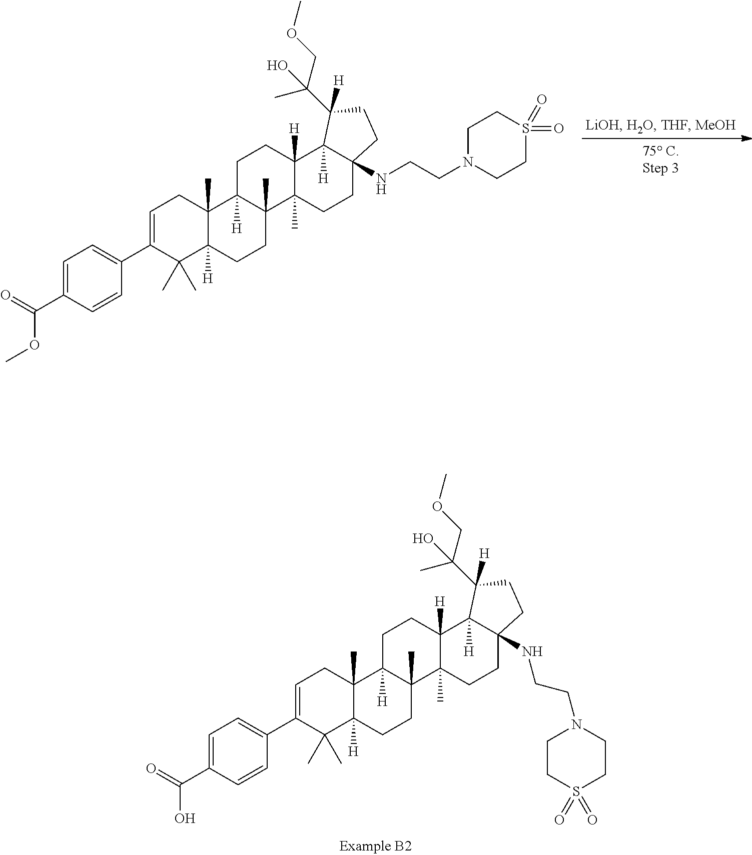

### Step 1: Preparation of methyl 4-((1R,3aS,5aR,5bR,7aR,11aS,11bR,13aR,13bS)-1-(1,2-dihydroxypropan-2-yl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 1 and methyl 4-01R,3aS,5aR,5bR,7aR,11aS,11bR,13aR,13bS)-1-(1,2-dihydroxypropan-2-yl)-3a-((2-(1,1-dioxidothiomorpholino)ethypamino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 2.

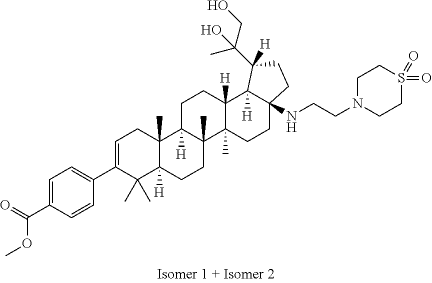

To a mixture of methyl 4-((1R,3aS,5aR,5bR,7aR,11aS,11bR,13aR,13bR)-3a-((2-(1,1-dioxido-4-thiomorpholinyl)ethyl)amino)-1-isopropenyl-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (1.11 g, 1.57 mmol), citric acid monohydrate (0.662 g, 3.15 mmol) and 4-methylmorpholine N-oxide (0.203 g, 1.73 mmol) was added tert-butanol (30 mL) followed by water (30 mL). Then osmium tetroxide, 2.5% in tert-butanol (0.800 g, 0.988 mL, 0.079 mmol) was added and the resulting mixture was stirred at rt for 27 hours. The olive green solution was concentrated in vacuo to a residue and was then redissolved in THF (150 mL). The solution was washed with brine (75 mL), then the organic was washed twice with a mixture of brine (50 mL) and 1N aqueous NaOH (10 mL), and then once more with brine (50 mL). The combined aqueous extracts were back-extracted with THF (75 mL) and the organic phases were combined. To the organic was added silica gel (11 g) and the mixture was concentrated in vacuo to a free-flowing powder which was placed in a vacuum oven at 50° C. for 16 h. The free flowing powder was loaded on the top of an 80 g silica gel column. Elution gradient 100% DCM to 9:1 DCM:MeOH gave separation of two diastereomers of the titled compound. The first of the two isomers of the titled compound to elute from the column was labeled Isomer 1 (0.232 g, 20% yield) and the second of the two isomers of the titled compound to elute from the column was labeled Isomer 2 (0.476 g, 41% yield). Analytical data for methyl 4-((1R,3aS,5aR,5bR,7aR,11aS,11bR,13aR,13bS)-1-(1,2-dihydroxypropan-2-yl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 1: LCMS: m/e 739.7 (M+H)⁺, 2.12 min (method 5). ¹H NMR (500 MHz, 1:1 CDCl₃:MeOD, MeOD lock) δ ppm 7.96-7.87 (m, J=8.1 Hz, 2H), 7.27-7.17 (m, J=8.1 Hz, 2H), 5.30 (d, J=4.6 Hz, 1H), 4.23 (s, 1H), 3.91 (s, 3H), 3.51 (d, J=11.2Hz, 1H), 3.19-3.08 (m, 6H), 3.08-2.96 (m, 2H), 2.80-2.63 (m, 2H), 2.63-2.50 (m, 1H), 2.50-2.35 (m, 1H), 2.23-2.05 (m, 2H), 1.98-1.86 (m, 2H), 1.86-1.61 (m, 6H), 1.60-1.40 (m, 7H), 1.39-1.24 (m, 4H), 1.17 (s, 3H), 1.15-1.08 (m, 1H), 1.05 (s, 3H), 1.04 (s, 3H), 1.01 (s, 3H), 0.95 (s, 3H), 0.94 (s, 3H). Analytical data for methyl 4-((1R,3aS,5aR,5bR,7aR,11aS,11bR,13aR,13bS)-1-(1,2-dihydroxypropan-2-yl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 2: LCMS: m/e 739.7 (M+H)⁺, 2.13 min (method 5). ¹H NMR (500 MHz, 1:1 CDCl₃:MeOD, MeOD lock) δ ppm 7.94-7.87 (m, J=8.3 Hz, 2H), 7.25-7.18 (m, J=8.3 Hz, 2H), 5.30 (d, J=4.9 Hz, 1H), 4.23 (s, 1H), 3.91 (s, 3H), 3.49 (d, J=10.8 Hz, 1H), 3.40 (d, J=11.0 Hz, 1H), 3.18-2.97 (m, 8H), 2.78-2.64 (m, 2H), 2.61-2.49 (m, 1H), 2.49-2.38 (m, 1H), 2.16 (dd, J=17.2, 6.2Hz, 1H), 2.06-1.98 (m, 1H), 1.98-1.91 (m, 1H), 1.91-1.83 (m, 1H), 1.83-1.62 (m, 7H), 1.60-1.32 (m, 8H), 1.31-1.24 (m, 2H), 1.18 (s, 3H), 1.16 (br. s., 3H), 1.15-1.06 (m, 2H), 1.04 (s, 3H), 1.01 (s, 3H), 0.95 (s, 3H), 0.94 (br. s., 3H).

### Step 2: Preparation of methyl 4-((1R,3aS,5aR,5bR,7aR,11aS,11bR,13aR,13bS)-3a-((2-(1,1-dioxidothiomorpholino)ethyl)amino)-1-(2-hydroxy-1-methoxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate

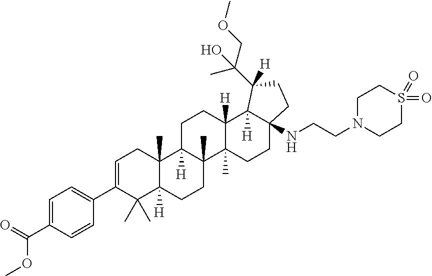

To a stirred mixture of methyl 4-((1R,3aS,5aR,5bR,7aR,11aS,11bR,13aR,13bS)-1-(1,2-dihydroxypropan-2-yl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 2 (0.020 g, 0.027 mmol) and 2,6-di-tert-butyl-4-methylpyridine (0.028 g, 0.135 mmol) in chloroform (0.5 mL) was added methyl trifluoromethanesulfonate (0.022 g, 0.135 mmol). The vial containing the solution was sealed and heated to 70° C. for 18 h. Additional methyl trifluoromethanesulfonate (0.022 g, 0.135 mmol) was added and the mixture was again heated to 70° C. for 30 min. The mixture was diluted with chloroform (2 mL) and washed with saturated aqueous ammonium chloride (3×1 mL). The organic phase was concentrated via nitrogen stream and purified by reverse phase preparative HPLC (Prep HPLC method 2) to provide the title compound (0.0094 g, 35.4% yield) as a bis-TFA salt. LCMS: m/e 753.8 (M+H)⁺, 2.31 min (method 5). ¹H NMR (400 MHz, acetone d6) δ ppm 8.00-7.90 (m, 2H), 7.35-7.25 (m, J=8.6 Hz, 2H), 5.34 (dd, J=6.1, 1.7 Hz, 1H), 3.89 (s, 3H), 3.43-3.35 (m, 3H), 3.35 (s, 3H), 3.32 (dd, J=7.5, 2.3 Hz, 2H), 3.29-3.24 (m, 2H), 3.24-3.11 (m, 6H), 3.11-3.01 (m, 2H), 2.45-2.36 (m, 1H), 2.35-2.27 (m, 1H), 2.27-2.18 (m, 2H), 2.18-2.11 (m, 1H), 2.00-1.92 (m, 3H), 1.88-1.79 (m, 2H), 1.79-1.70 (m, 2H), 1.69-1.53 (m, 6H), 1.53-1.41 (m, 4H), 1.36 (s, 3H), 1.32 (d, J=11.0 Hz, 2H), 1.19 (s, 3H), 1.17 (s, 3H), 1.09 (s, 3H), 1.00 (s, 3H), 0.98 (s, 3H).

Step 3: A mixture of methyl 4-((1R,3aS,5aR,5bR,7aR,11aS,11bR,13aR,13bS)-3a-((2-(1,1-dioxidothiomorpholino)ethyl)amino)-1-(2-hydroxy-1-methoxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (0.094 g, 0.0096 mmol), lithium hydroxide monohydrate (0.0040 g, 0.096 mmol), methanol (0.3 mL), THF (0.3 mL) and water (0.3 mL) was heated with stirring to 75° C. for 80 min. The crude mixture was purified by reverse phase preparative HPLC (Prep HPLC method 3) to provide the title compound (0.0104 g, >100% yield) as a bis-TFA salt.

LCMS: m/e 739.6 (M+H)⁺, 2.08 min (method 5). ¹H NMR (400 MHz, acetone d6) δ ppm 8.03-7.92 (m, J=8.3 Hz, 2H), 7.34-7.25 (m, J=8.3 Hz, 2H), 5.39-5.29 (m, 1H), 4.05 (s, 1H), 3.42-3.36 (m, 2H), 3.35 (s, 3H), 3.33-3.29 (m, 2H), 3.29-3.24 (m, 2H), 3.24-3.12 (m, 6H), 3.11-3.02 (m, 2H), 2.44-2.36 (m, 1H), 2.36-2.27 (m, 1H), 2.27-2.10 (m, 3H), 2.01-1.89 (m, 3H), 1.88-1.79 (m, 2H), 1.79-1.70 (m, 2H), 1.69-1.53 (m, 6H), 1.53-1.42 (m, 4H), 1.36 (s, 3H), 1.32 (d, J=12.7 Hz, 2H), 1.19 (s, 3H), 1.17 (s, 3H), 1.10 (s, 3H), 1.00 (s, 3H), 0.98 (s, 3H).

### Example B2

### Preparation of 4-((1R,3aS,5aR,5bR,7aR,11aS,11bR,13aR,13bS)-1-(1,2-dimethoxypropan-2-yl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid

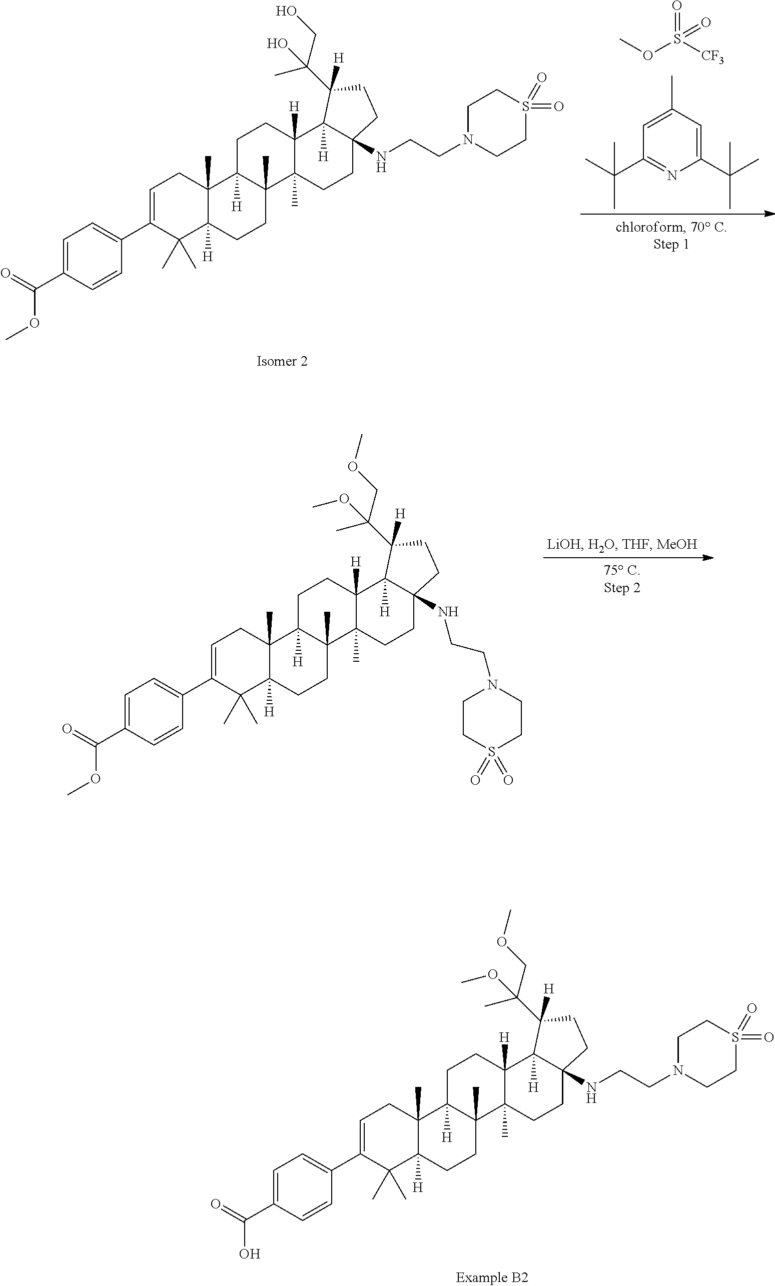

### Step 1: Preparation of methyl 4-((1R,3aS,5aR,5bR,7aR,11aS,11bR,13aR,13bS)-1-(1,2-dimethoxypropan-2-yl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate

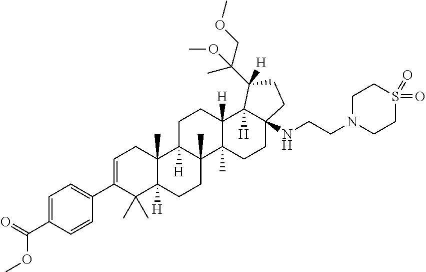

To a stirred mixture of methyl 4-((1R,3aS,5aR,5bR,7aR,11aS,11bR,13aR,13bS)-1-(1,2-dihydroxypropan-2-yl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 2 (0.020 g, 0.027 mmol) and 2,6-di-tert-butyl-4-methylpyridine (0.056 g, 0.271 mmol) in chloroform (0.5 mL) was added methyl trifluoromethanesulfonate (0.058 g, 0.352 mmol). The vial containing the solution was sealed and heated to 70° C. for 4 h. The mixture was diluted with chloroform (1.5 mL) and washed with saturated aqueous ammonium chloride (1 mL). The aqueous wash was back-extracted with chloroform (2×1 mL) and the organic phases were combined. The combined organic phase was concentrated via nitrogen stream and purified by reverse phase preparative HPLC (Prep HPLC method 2) to provide the title compound (0.010 g, 48.2% yield). LCMS: m/e 767.7 (M+H)⁺, 2.34 min (method 5). ¹H NMR (400 MHz, acetone d6) δ ppm 8.00-7.90 (m, J=8.3 Hz, 2H), 7.35-7.25 (m, J=8.3 Hz, 2H), 5.35 (dd, J=6.0, 1.3 Hz, 1H), 3.90 (s, 3H), 3.40 (s, 2H), 3.39-3.34 (m, 2H), 3.33 (s, 3H), 3.32-3.26 (m, 1H), 3.22 (br. s., 3H), 3.19 (s, 4H), 3.17-3.12 (m, 2H), 3.12-3.01 (m, 2H), 2.65-2.55 (m, 1H), 2.36 (dd, J=12.0, 8.8 Hz, 1H), 2.28-2.17 (m, 2H), 2.15-2.09 (m, 1H), 2.03-1.92 (m, 2H), 1.91-1.79 (m, 3H), 1.78 (s, 1H), 1.76-1.68 (m, 1H), 1.66-1.54 (m, 6H), 1.54-1.38 (m, 4H), 1.34 (s, 4H), 1.31 (br. s., 1H), 1.15 (s, 3H), 1.15 (s, 3H), 1.09 (s, 3H), 1.00 (s, 3H), 0.98 (s, 3H).

### Step 2: Preparation of 4-((1R,3aS,5aR,5bR,7aR,11aS,11bR,13aR,13bS)-1-(1,2-dimethoxypropan-2-yl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid

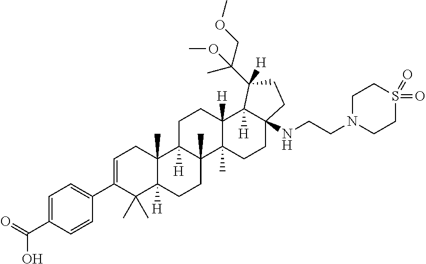

A mixture of methyl 4-((1R,3aS,5aR,5bR,7aR,11aS,11bR,13aR,13bS)-1-(1,2-dimethoxypropan-2-yl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (0.0155 g, 0.016 mmol), lithium hydroxide monohydrate (0.0065 g, 0.156 mmol), methanol (0.3 mL), THF (0.3 mL) and water (0.3 mL) was heated with stirring to 75° C. for 80 min. The crude mixture was purified by reverse phase preparative HPLC (Prep HPLC method 3) to provide the title compound (0.0118 g, 76% yield). LCMS: m/e 753.6 (M+H)⁺, 2.19 min (method 5). ¹H NMR (400 MHz, acetone d6) δ ppm 7.98 (d, J=8.3 Hz, 2H), 7.30 (d, J=8.1 Hz, 2H), 5.35 (d, J=4.9 Hz, 1H), 3.43-3.25 (m, 9H), 3.25-3.11 (m, 9H), 3.11-2.98 (m, 2H), 2.58 (t, J=8.4 Hz, 1H), 2.35 (dd, J=11.9, 8.7 Hz, 1H), 2.29-2.16 (m, 2H), 2.16-2.09 (m, 1H), 2.03-1.90 (m, 2H), 1.90-1.84 (m, 1H), 1.80 (d, J=18.1 Hz, 3H), 1.75-1.67 (m, 1H), 1.60 (dd, J=15.9, 7.8 Hz, 6H), 1.54-1.38 (m, 4H), 1.35 (s, 4H), 1.31 (br. s., 1H), 1.17-1.12 (m, 6H), 1.10 (s, 3H), 1.00 (s, 3H), 0.99 (s, 3H).

### Example B3

### Preparation of 4-((1R,3aS,5aR,5bR,7aR,11aS,11bR,13aR,13bS)-3a-((2-(1,1-dioxidothiomorpholino)ethyl)amino)-1-(2-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid.

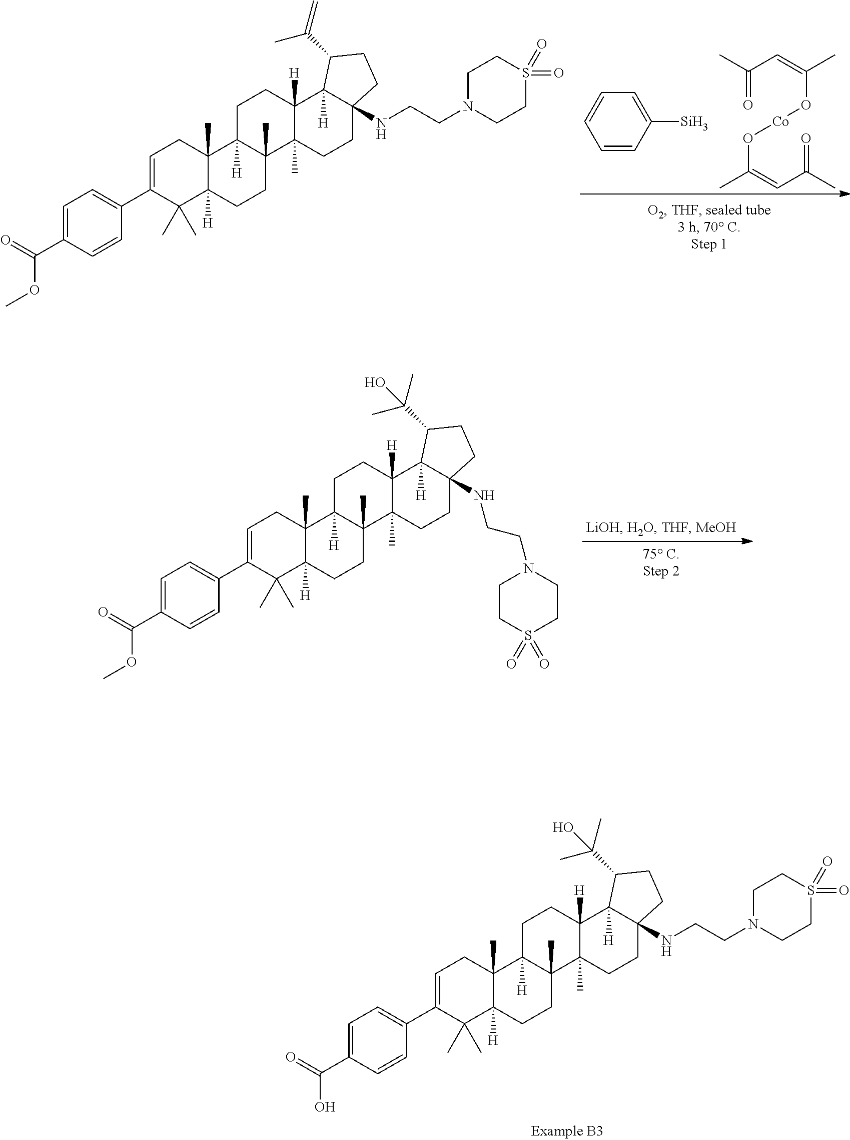

### Step 1: Preparation of methyl 4-((1R,3aS,5aR,5bR,7aR,11aS,11bR,13aR,13bS)-3a-((2-(1,1-dioxidothiomorpholino)ethyl)amino)-1-(2-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate.

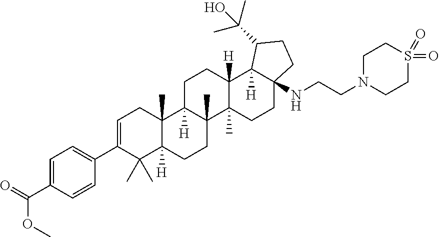

To a stirred mixture of methyl 4-((1R,3aS,5aR,5bR,7aR,11aS,11bR,13aR,13bR)-3a-((2-(1,1-dioxido-4-thiomorpholinyl)ethyl)amino)-1-isopropenyl-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (0.050 g, 0.071 mmol) and cobalt (II) acetylacetonate (3.7 mg, 0.014 mmol) in dry THF (1 mL) was added phenylsilane (0.015 g, 0.142 mmol). The mixture was blanketed with oxygen gas and the vial was sealed and the mixture heated to 70° C. for 69 h. The mixture was removed from heat. Additional cobalt (II) acetylacetonate (24 mg, 0.11 mmol) and phenylsilane (0.015 g, 0.142 mmol) were added, the vial was fitted with a balloon of oxygen gas, and the mixture was stirred at rt for 3 h. Purification by reverse phase preparative HPLC (Prep HPLC method 4) provided the title compound (0.0196 g, 29% yield) as a bis-TFA salt. LCMS: m/e 723.6 (M+H)⁺, 2.20 min (method 5). ¹H NMR (400 MHz, CDC/₃) δ ppm 8.00-7.92 (m, J=8.3 Hz, 2H), 7.26-7.17 (m, J=8.3 Hz, 2H), 5.32 (d, J=4.6 Hz, 1H), 3.93 (s, 3H), 3.35-3.10 (m, 8H), 3.10-2.99 (m, 2H), 2.93 (d, J=13.2Hz, 1H), 2.89-2.77 (m, 1H), 2.28-2.12 (m, 2H), 2.12-1.91 (m, 4H), 1.87 (dd, J=13.8, 5.3 Hz, 1H), 1.83-1.76 (m, 1H), 1.76-1.63 (m, 4H), 1.63-1.34 (m, 11H), 1.29 (s, 4H), 1.25 (s, 4H), 1.18 (s, 3H), 1.12 (s, 3H), 1.04 (s, 3H), 0.97 (s, 3H), 0.95 (s, 3H).

Step 2: A mixture of methyl 4-((1R,3aS,5aR,5bR,7aR,11aS,11bR,13aR,13bS)-3a-((2-(1,1-dioxidothiomorpholino)ethyl)amino)-1-(2-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (0.0195 g, 0.021 mmol), lithium hydroxide monohydrate (0.0103 g, 0.246 mmol), methanol (0.3 mL), THF (0.3 mL) and water (0.3 mL) was heated with stirring to 75° C. for 80 min. The crude mixture was purified by reverse phase preparative HPLC (Prep HPLC method 3) to provide the title compound (0.0186 g, 96% yield) as a bis-TFA salt. LCMS: m/e 709.6 (M+H)⁺, 2.01 min (method 5). ¹H NMR (400 MHz, 1:1 CDCl₃:MeOD, MeOD lock) δ ppm 7.96-7.89 (m, J=8.3 Hz, 2H), 7.24-7.16 (m, J=8.3 Hz, 2H), 5.30 (d, J=4.6 Hz, 1H), 3.98 (s, 1H), 3.37 (s, 1H), 3.32-3.14 (m, 6H), 3.14-2.99 (m, 6H), 2.23-2.11 (m, 2H), 2.11-1.99 (m, 2H), 1.95-1.81 (m, 4H), 1.81-1.68 (m, 3H), 1.67-1.53 (m, 5H), 1.53-1.36 (m, 6H), 1.31-1.25 (m, 2H), 1.24 (s, 3H), 1.22 (s, 3H), 1.17 (s, 3H), 1.14 (s, 3H), 1.04 (s, 3H), 0.96 (s, 3H), 0.95 (br. s., 3H).

### Example B4

### Preparation of 4-((1R,3aS,5aR,5bR,7aR,11aS,11bR,13aR,13bS)-3a-((2-(1,1-dioxidothiomorpholino)ethyl)amino)-1-(2-methoxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid.

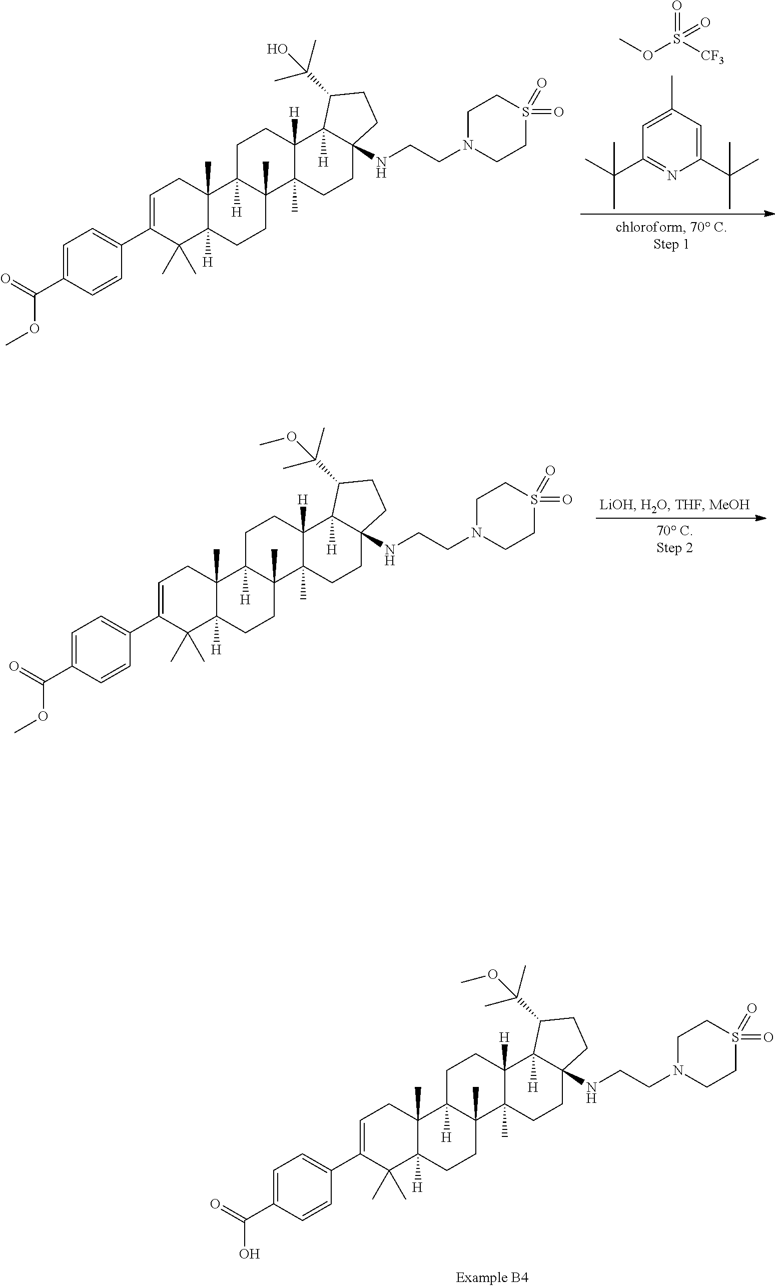

### Step 1: Preparation of methyl 4-((1R,3aS,5aR,5bR,7aR,11aS,11bR,13aR,13bS)-3a-((2-(1,1-dioxidothiomorpholino)ethyl)amino)-1-(2-methoxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate.

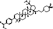

To a stirred mixture of methyl 4-((1R,3aS,5aR,5bR,7aR,11aS,11bR,13aR,13bS)-3a-((2-(1,1-dioxidothiomorpholino)ethyl)amino)-1-(2-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (0.020 g, 0.021 mmol) and 2,6-di-tert-butyl-4-methylpyridine (0.043 g, 0.210 mmol) in chloroform (0.5 mL) was added methyl trifluoromethanesulfonate (0.035 g, 0.210 mmol). The vial containing the solution was sealed and heated to 70° C. for 80 min. The crude mixture was concentrated via nitrogen stream and purified by reverse phase preparative HPLC (Prep HPLC method 4) to provide the title compound (0.0164 g, 81% yield) as a bis-TFA salt. LCMS: m/e 737.7 (M+H)⁺, 2.34 min (method 5). ¹H NMR (400 MHz, acetone-d₆) δ ppm 7.99-7.91 (m, J=8.3 Hz, 2H), 7.34-7.27 (m, J=8.3 Hz, 2H), 5.34 (dd, J=6.1, 1.7 Hz, 1H), 3.90 (s, 3H), 3.43-3.27 (m, 4H), 3.27-3.15 (m, 6H), 3.13 (s, 3H), 3.11-3.01 (m, 2H), 2.44 (t, J=8.1 Hz, 1H), 2.27-2.18 (m, 2H), 2.18-2.09 (m, 2H), 2.04-1.95 (m, 2H), 1.89-1.80 (m, 3H), 1.80-1.71 (m, 2H), 1.67-1.53 (m, 6H), 1.53-1.44 (m, 3H), 1.41 (d, J=11.2Hz, 1H), 1.38-1.28 (m, 5H), 1.17-1.14 (m, 6H), 1.13 (s, 3H), 1.09 (s, 3H), 1.00 (s, 3H), 0.98 (s, 3H).

Step 2: A mixture of methyl 4-((1R,3aS,5aR,5bR,7aR,11aS,11bR,13aR,13bS)-3a-((2-(1,1-dioxidothiomorpholino)ethyl)amino)-1-(2-methoxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (0.0162 g, 0.017 mmol), lithium hydroxide monohydrate (0.0084 g, 0.201 mmol), methanol (0.35 mL), THF (0.35 mL) and water (0.2 mL) was heated with stirring to 70° C. for 20 min. The crude mixture was purified by reverse phase preparative HPLC (Prep HPLC method 5) to provide the title compound (0.0151 g, 91% yield) as a bis-TFA salt. LCMS: m/e 723.7 (M+H)⁺, 2.18 min (method 5). ¹H NMR (400 MHz, acetone-d6) δ ppm 8.04-7.90 (m, J=8.3 Hz, 2H), 7.35-7.24 (m, J=8.3 Hz, 2H), 5.35 (d, J=4.6 Hz, 1H), 3.43-3.26 (m, 4H), 3.26-3.21 (m, 3H), 3.18 (d, J=9.0 Hz, 3H), 3.13 (s, 3H), 3.11-3.00 (m, 2H), 2.46 (t, J=7.9 Hz, 1H), 2.29-2.19 (m, 2H), 2.19-2.11 (m, 2H), 2.03-1.88 (m, 3H), 1.88-1.70 (m, 6H), 1.67-1.44 (m, 9H), 1.41 (d, J=11.0 Hz, 1H), 1.35 (s, 3H), 1.34-1.28 (m, 2H), 1.15 (s, 6H), 1.13 (s, 3H), 1.10 (s, 3H), 1.00 (s, 3H), 0.99 (s, 3H).

### Example B5

### Preparation of 4-((1R,3aS,5aR,5bR,7aR,11aS,11bR,13aR,13bR)-3a-((2-(1,1-dioxidothiomorpholino)ethyl)amino)-1-(1-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid Isomer 1.

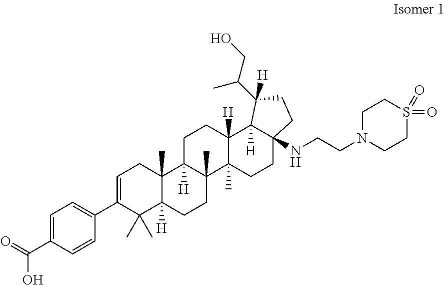

A solution of methyl 4-((1R,3aS,5aR,5bR,7aR,11aS,11bR,13aR,13bR)-3a-((2-(1,1-dioxido-4-thiomorpholinyl)ethyl)amino)-1-isopropenyl-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (0.100 g, 0.142 mmol) in THF (1 mL) was chilled in an ice bath and was treated slowly with borane-tetrahydrofuran complex, 1.0M in THF (0.340 mL, 0.340 mmol). The mixture was stirred at rt for 16 h, then at 70° C. for 30 min. The mixture was again chilled in an ice bath and to it was added ethanol (0.180 mL, 3.1 mmol) and saturated aqueous sodium acetate (0.066 mL), followed by slow addition of 30% hydrogen peroxide (0.092 mL, 0.90 mmol). The resulting mixture was stirred at rt for 3 h. The mixture was purified by reverse phase preparative HPLC (Prep HPLC method 4) to provided a white solid (56.7 mg) which contained the major isomer product of the reaction. The minor isomer was not isolated. A portion of this solid (20.0 mg, 35.3% of the total recovered) was dissolved in a mixture of MeOH (0.25 mL) and THF (0.25 mL) and treated with 1.0M aqueous LiOH (0.252 mL, 0.252 mmol) at 70° C. for 45 min. Purification of this mixture by reverse phase preparative HPLC (Prep HPLC method 4) followed by treatment of the product fraction with 1M aqueous HCl and subsequent concentration in vacuo provided the Isomer 1 title compound bis HCl salt as a white solid (0.0138 g, 33.9% overall yield). LCMS: m/e 709.7 (M+H)⁺, 1.95 min (method 5). ¹H NMR (400 MHz, 1:1 CDCl₃:MeOD, MeOD lock) δ ppm 7.96-7.88 (m, J=8.3 Hz, 2H), 7.24-7.17 (m, J=8.1 Hz, 2H), 5.31 (d, J=4.6 Hz, 1H), 4.28 (br. s., 3H), 3.65 (dd, J=10.9, 6.2Hz, 1H), 3.46 (dd, J=11.1, 6.2Hz, 1H), 3.31-3.24 (m, 3H), 3.23-3.13 (m, 2H), 3.13-2.99 (m, 5H), 2.42-2.25 (m, 2H), 2.17 (dd, J=17.2, 6.2Hz, 1H), 2.09-2.01 (m, 1H), 2.00-1.91 (m, 2H), 1.90-1.78 (m, 3H), 1.78-1.67 (m, 3H), 1.66-1.38 (m, 11H), 1.33-1.23 (m, 2H), 1.20 (s, 3H), 1.09 (s, 3H), 1.04 (s, 3H), 0.99-0.90 (m, 9H).

### Example B6

### Preparation of 4-((1R,3aS,5aR,5bR,7aR,11aS,11bR,13aR,13bR)-3a-((2-(1,1-dioxidothiomorpholino)ethyl)amino)-1-(1-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid Isomer 2

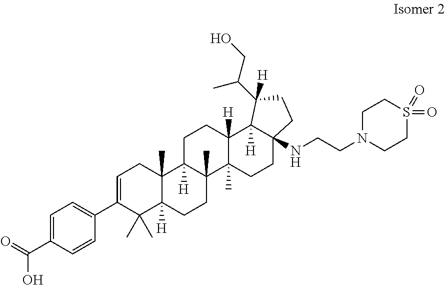

A solution of methyl 4-((1R,3aS,5aR,5bR,7aR,11aS,11bR,13aR,13bR)-3a-((2-(1,1-dioxido-4-thiomorpholinyl)ethyl)amino)-1-isopropenyl-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (1.00 g, 1.42 mmol) in THF (10 mL) was chilled in an ice bath and was treated slowly with borane-tetrahydrofuran complex, 1.0M in THF (3.40 mL, 3.40 mmol). The mixture was stirred at rt for 16 h, then at 70° C. for 30 min. The mixture was again chilled in an ice bath and to it was added ethanol (0.90 mL, 15.4 mmol) and saturated aqueous sodium acetate (0.33 mL), followed by slow addition of 30% hydrogen peroxide (0.46 mL, 4.5 mmol). The resulting mixture was stirred at rt for 2.5 h. To the organic was added silica gel (11 g) and the mixture was concentrated in vacuo to a free-flowing powder which was placed in a vacuum oven at 50° C. for 16 h. The free flowing powder was loaded on the top of an 160 g silica gel column. Elution gradient 100% DCM to 9:1 DCM:MeOH gave separation of two diastereomers. The major isomer from the reaction methyl 4-((1R,3aS,5aR,5bR,7aR,11aS,11bR,13aR,13bR)-3a-((2-(1,1-dioxidothiomorpholino)ethyl)amino)-1-(1-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 1 eluted from the column first (0.458 g, 44.6% yield). LCMS: m/e 723.6 (M+H)⁺, 2.14 min (method 5). ¹H NMR (400 MHz, 1:1 CDCl₃:MeOD, MeOD lock) δ ppm 7.94-7.88 (m, J=8.3 Hz, 2H), 7.25-7.18 (m, J=8.3 Hz, 2H), 5.33-5.26 (m, 1H), 4.25 (br. s., 1H), 3.91 (s, 3H), 3.74 (dd, J=10.5, 4.2Hz, 1H), 3.17-2.95 (m, 9H), 2.78-2.62 (m, 2H), 2.62-2.52 (m, 1H), 2.47-2.38 (m, 1H), 2.14 (dd, J=17.1, 6.4 Hz, 1H), 2.00-1.80 (m, 4H), 1.80-1.71 (m, 2H), 1.71-1.48 (m, 10H), 1.48-1.32 (m, 5H), 1.30-1.18 (m, 3H), 1.15 (s, 3H), 1.09 (d, J=14.9 Hz, 2H), 1.01 (s, 6H), 0.98 (d, J=6.8 Hz, 3H), 0.95 (s, 3H), 0.94 (s, 3H). The minor of the two isomers formed in the reaction eluted from the column after the major isomer and was mixed with impurities. These impure fractions containing the minor isomer were repurified by reverse phase preparative HPLC (Prep HPLC method 6) to provide a solid (53.8 mg). A portion of this solid (25 mg, 46.5% of the total) was dissolved in a mixture of MeOH (0.3 mL) and THF (0.3 mL) and treated with 1.0M aqueous LiOH (0.263 mL, 0.263 mmol) at 70° C. for 45 min. Purification of this mixture by reverse phase preparative HPLC (Prep HPLC method 7) provided the Isomer 2 title compound as a white solid free base (0.0115 g, 5.2% overall yield). LCMS: m/e 710.5 (M+H)⁺, 2.18 min (method 5). ¹H NMR (400 MHz, 1:1 CDCl₃:MeOD, MeOD lock) δ ppm 7.86 (d, J=8.1 Hz, 2H), 7.13 (d, J=8.3 Hz, 2H), 5.27 (d, J=4.6 Hz, 1H), 3.43-3.35 (m, 2H), 3.18-2.97 (m, 8H), 2.80-2.65 (m, 2H), 2.65-2.54 (m, 1H), 2.54-2.43 (m, 1H), 2.12 (dd, J=16.9, 5.9 Hz, 2H), 1.94 (s, 4H), 1.92-1.84 (m, 2H), 1.79 (dd, J=12.7, 7.1 Hz, 1H), 1.75-1.66 (m, 2H), 1.65-1.36 (m, 11H), 1.35-1.20 (m, 4H), 1.15 (s, 3H), 1.11 (d, J=13.9 Hz, 1H), 1.02 (s, 6H), 0.94 (s, 6H), 0.81 (d, J=6.8 Hz, 3H).

### Example B7

### Preparation of 4-((1R,3aS,5aR,5bR,7aR,11aS,11bR,13aR,13bR)-3a-((2-(1,1-dioxidothiomorpholino)ethyl)amino)-1-(1-methoxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid

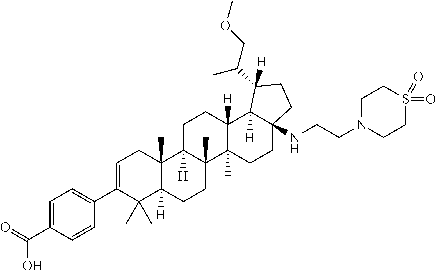

A solution of 4-((1R,3aS,5aR,5bR,7aR,11aS,11bR,13aR,13bR)-3a-((2-(1,1-dioxido-4-thiomorpholinyl)ethyl)amino)-1-isopropenyl-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (0.100 g, 0.142 mmol) in THF (1 mL) was chilled in an ice bath and was treated slowly with borane-tetrahydrofuran complex, 1.0M in THF (0.340 mL, 0.340 mmol). The mixture was stirred at rt for 16 h, then at 70° C. for 30 min. The mixture was again chilled in an ice bath and to it was added ethanol (0.180 mL, 3.1 mmol) and saturated aqueous sodium acetate (0.066 mL), followed by slow addition of 30% hydrogen peroxide (0.092 mL, 0.90 mmol). The resulting mixture was stirred at rt for 3 h. The mixture was purified by reverse phase preparative HPLC (Prep HPLC method 6) to provided a white solid (56.7 mg) which contained the major isomer product of the reaction. The minor isomer was not isolated. A portion of this solid (30.0 mg, 52.9% of the total recovered) was dissolved in chloroform (0.5 mL) and treated with 2,6-di-tert-butyl-4-methylpyridine (0.065 g, 0.315 mmol) and methyl trifluoromethanesulfonate (0.052 g, 0.315 mmol). The mixture was stirred at rt for 16 h, then at 70° C. for 30 min. Purification of this mixture by reverse phase preparative HPLC provided (Prep HPLC method 3) the title compound bis TFA salt as a colorless glassy solid (0.0177 g, 24.0% overall yield). LCMS: m/e 737.7 (M+H)⁺, 2.34 min (method 5). ¹H NMR (400 MHz, acetone d6) δ ppm 8.03-7.93 (m, J=8.3 Hz, 2H), 7.35-7.26 (m, J=8.1 Hz, 2H), 5.40-5.30 (m, 1H), 3.50 (dd, J=9.5, 6.8 Hz, 1H), 3.36-3.06 (m, 17H), 2.45-2.28 (m, 2H), 2.28-2.19 (m, 2H), 2.19-2.12 (m, 2H), 1.96-1.69 (m, 6H), 1.69-1.39 (m, 12H), 1.39-1.34 (m, 1H), 1.33 (s, 4H), 1.13 (s, 3H), 1.10 (s, 3H), 1.01 (s, 3H), 0.99 (s, 3H), 0.90 (d, J=6.8 Hz, 3H).

### Example B8

### Preparation of 4-((1R,3aS,5aR,5bR,7aR,11aS,11bR,13aR,13bR)-3a-((2-(1,1-dioxidothiomorpholino)ethyl)amino)-1-(1-fluoropropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid.

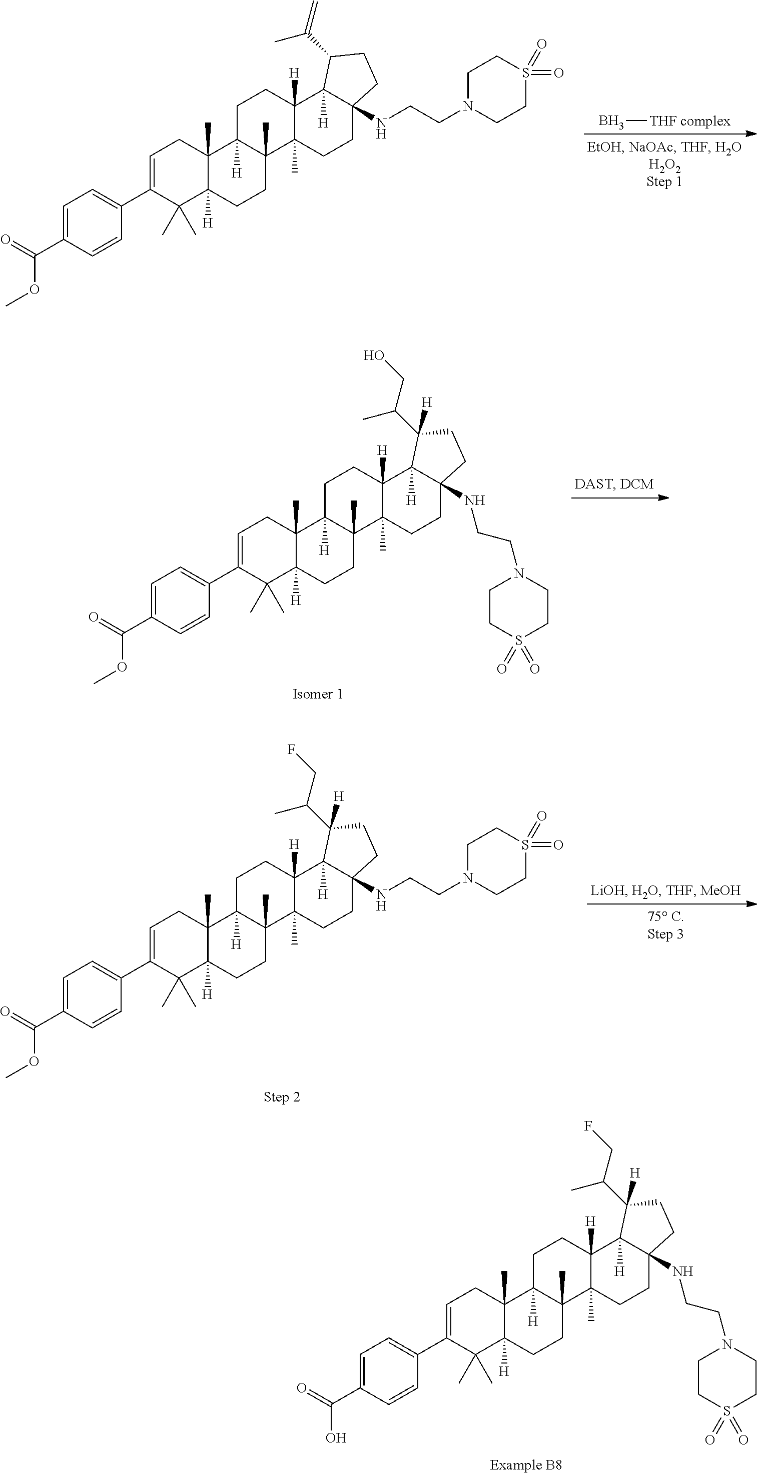

### Step 1: Preparation of methyl 4-((1R,3aS,5aR,5bR,7aR,11aS,11bR,13aR,13bR)-3a-((2-(1,1-dioxidothiomorpholino)ethyl)amino)-1-(1-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 1.

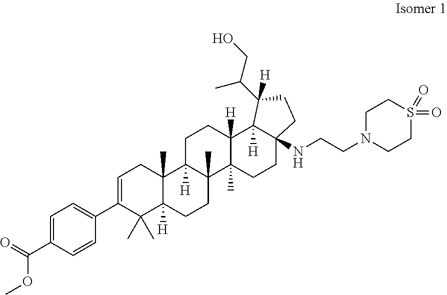

A solution of 4-((1R,3aS,5aR,5bR,7aR,11aS,11bR,13aR,13bR)-3a-((2-(1,1-dioxido-4-thiomorpholinyl)ethyl)amino)-1-isopropenyl-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (1.00 g, 1.42 mmol) in THF (10 mL) was chilled in an ice bath and was treated slowly with borane-tetrahydrofuran complex, 1.0M in THF (3.40 mL, 3.40 mmol). The mixture was stirred at rt for 16 h, then at 70° C. for 30 min. The mixture was again chilled in an ice bath and to it was added ethanol (0.90 mL, 15.4 mmol) and saturated aqueous sodium acetate (0.33 mL), followed by slow addition of 30% hydrogen peroxide (0.46 mL, 4.5 mmol). The resulting mixture was stirred at rt for 2.5 h. To the organic was added silica gel (11 g) and the mixture was concentrated in vacuo to a free-flowing powder which was placed in a vacuum oven at 50° C. for 16 h. The free flowing powder was loaded on the top of an 160 g silica gel column. Elution gradient 100% DCM to 9:1 DCM:MeOH gave separation of two diastereomers. The major isomer from the reaction methyl 4-((1R,3aS,5aR,5bR,7aR,11aS,11bR,13aR,13bR)-3a-((2-(1,1-dioxidothiomorpholino)ethyl)amino)-1-(1-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 1 eluted from the column first (0.458 g, 44.6% yield). LCMS: m/e 723.6 (M+H)⁺, 2.14 min (method 5). ¹H NMR (400 MHz, 1:1 CDCl₃:MeOD, MeOD lock) δ ppm 7.94-7.88 (m, J=8.3 Hz, 2H), 7.25-7.18 (m, J=8.3 Hz, 2H), 5.33-5.26 (m, 1H), 4.25 (br. s., 1H), 3.91 (s, 3H), 3.74 (dd, J=10.5, 4.2Hz, 1H), 3.17-2.95 (m, 9H), 2.78-2.62 (m, 2H), 2.62-2.52 (m, 1H), 2.47-2.38 (m, 1H), 2.14 (dd, J=17.1, 6.4 Hz, 1H), 2.00-1.80 (m, 4H), 1.80-1.71 (m, 2H), 1.71-1.48 (m, 10H), 1.48-1.32 (m, 5H), 1.30-1.18 (m, 3H), 1.15 (s, 3H), 1.09 (d, J=14.9 Hz, 2H), 1.01 (s, 6H), 0.98 (d, J=6.8 Hz, 3H), 0.95 (s, 3H), 0.94 (s, 3H).

### Step 2: Preparation of methyl 4-((1R,3aS,5aR,5bR,7aR,11aS,11bR,13aR,13bR)-3a-((2-(1,1-dioxidothiomorpholino)ethyl)amino)-1-(1-fluoropropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate

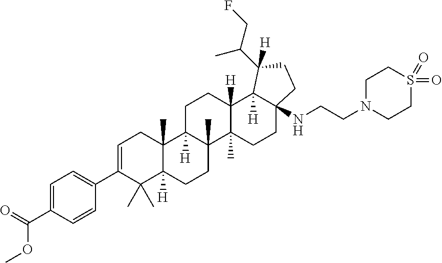

A solution of methyl 4-((1R,3aS,5aR,5bR,7aR,11aS,11bR,13aR,13bR)-3a-((2-(1,1-dioxidothiomorpholino)ethyl)amino)-1-(1-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 1 (0.025 g, 0.035 mmol) in DCM (1 mL) was cooled to −78° C. and was treated with DAST (0.0078 g, 0.048 mmol). The mixture was allowed to warm to rt and was stirred for 21 h. The reaction mixture was concentrated and purified by reverse phase preparative HPLC (Prep HPLC method 8) to provide the title compound (0.0097 g, 29% yield) as a bis-TFA salt. LCMS: m/e 725.6 (M+H)⁺, 2.35 min (method 5). ¹H NMR (400 MHz, CDC/₃) δ ppm 8.05-7.87 (m, J=8.1 Hz, 2H), 7.26-7.12 (m, J=8.1 Hz, 2H), 5.32 (d, J=4.9 Hz, 1H), 4.58-4.28 (m, 2H), 3.93 (s, 3H), 3.39-3.11 (m, 8H), 3.11-2.98 (m, 2H), 2.91 (d, J=12.0 Hz, 2H), 2.38-2.22 (m, 2H), 2.22-2.11 (m, 2H), 1.95 (d, J=10.5Hz, 4H), 1.84-1.61 (m, 5H), 1.55 (s, 2H), 1.57 (s, 3H), 1.51-1.31 (m, 6H), 1.31-1.15 (m, 5H), 1.06 (s, 3H), 1.04 (s, 3H), 0.97 (br. s., 3H), 0.95 (br. s., 3H), 0.91 (d, J=6.6 Hz, 3H).

Step 3: A mixture of methyl 4-((1R,3aS,5aR,5bR,7aR,11aS,11bR,13aR,13bR)-3a-((2-(1,1-dioxidothiomorpholino)ethyl)amino)-1-(1-fluoropropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (0.0095 g, 0.010 mmol), 1.0M aqueous lithium hydroxide monohydrate (0.100 mL, 0.100 mmol), methanol (0.3 mL) and THF (0.3 mL) was heated with stirring to 70° C. for 45 min. The crude mixture was purified by reverse phase preparative HPLC (Prep HPLC method 2) to provide the title compound (0.0152 g, >100% yield) as a bis-TFA salt. LCMS: m/e 711.4 (M+H)⁺, 2.19 min (method 5). ¹H NMR (400 MHz, 1:1 CDCl₃:MeOD, MeOD lock) δ ppm 7.96-7.88 (m, J=8.3 Hz, 2H), 7.25-7.17 (m, J=8.1 Hz, 2H), 5.31 (d, J=4.6 Hz, 1H), 4.49-4.38 (m, 1H), 3.30-3.00 (m, 13H), 2.33-2.21 (m, 2H), 2.21-2.11 (m, 2H), 2.11-2.03 (m, 2H), 2.02-1.95 (m, 1H), 1.94-1.78 (m, 3H), 1.76 (br. s., 1H), 1.74-1.65 (m, 2H), 1.65-1.55 (m, 4H), 1.52 (dd, J=14.1, 3.1 Hz, 3H), 1.47 (br. s., 3H), 1.45-1.37 (m, 2H), 1.32-1.24 (m, 2H), 1.21 (s, 3H), 1.07 (s, 3H), 1.05 (s, 3H), 0.96 (s, 3H), 0.95 (s, 3H), 0.93 (d, J=6.8 Hz, 3H).

### Example B9

### Preparation of 4-((1R,3aS,5aR,5bR,7aR,11aS,11bR,13aR,13bS)-1-(1,2-dihydroxypropan-2-yl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid, Isomer 1

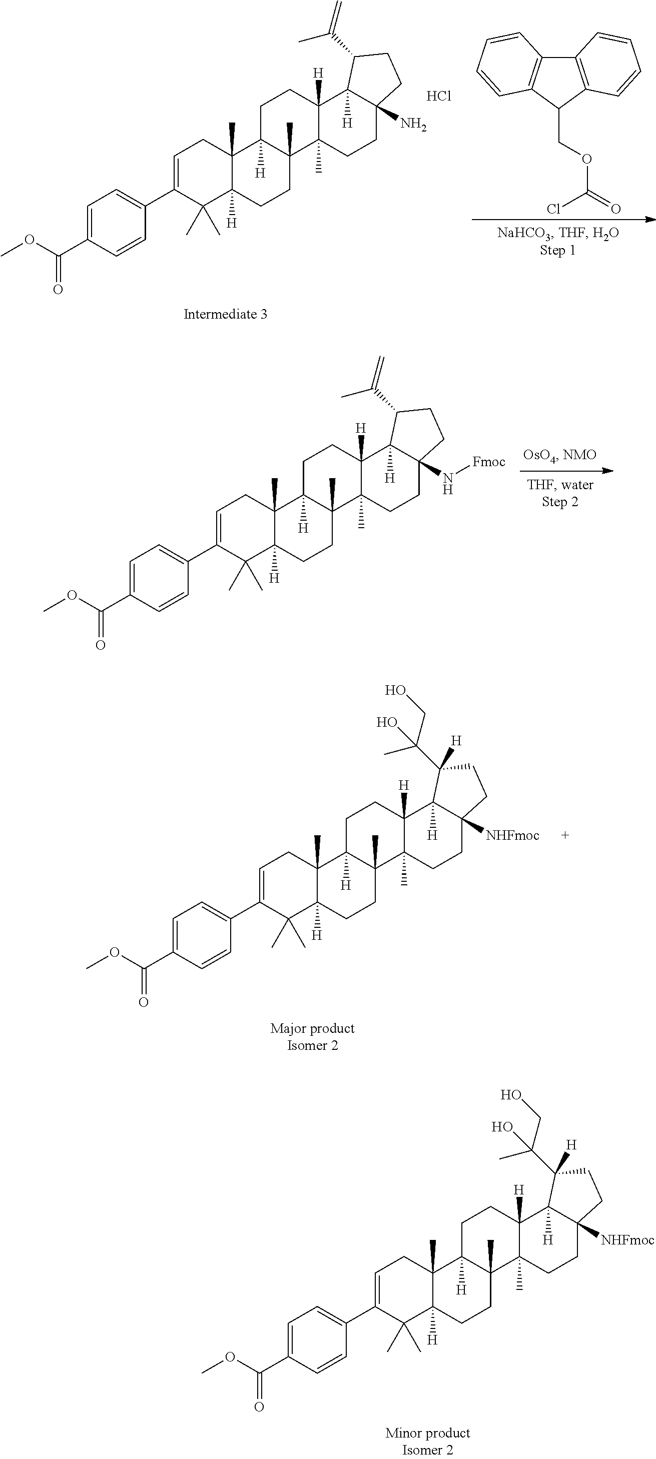

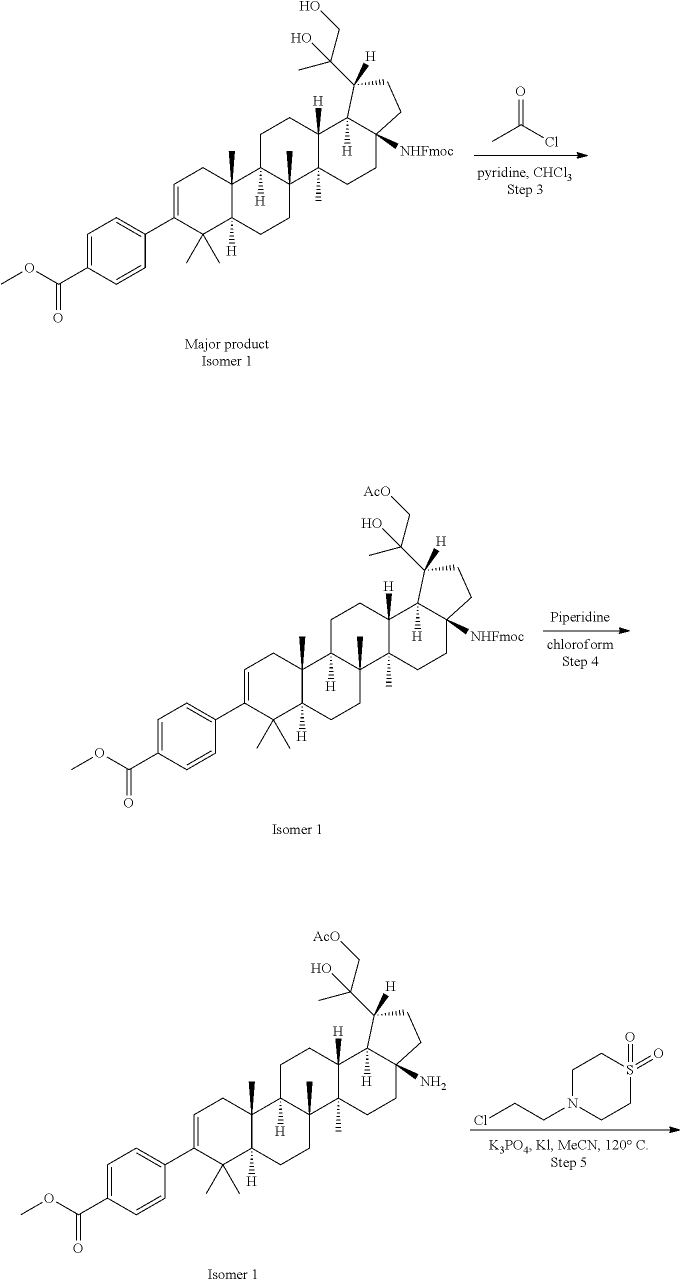

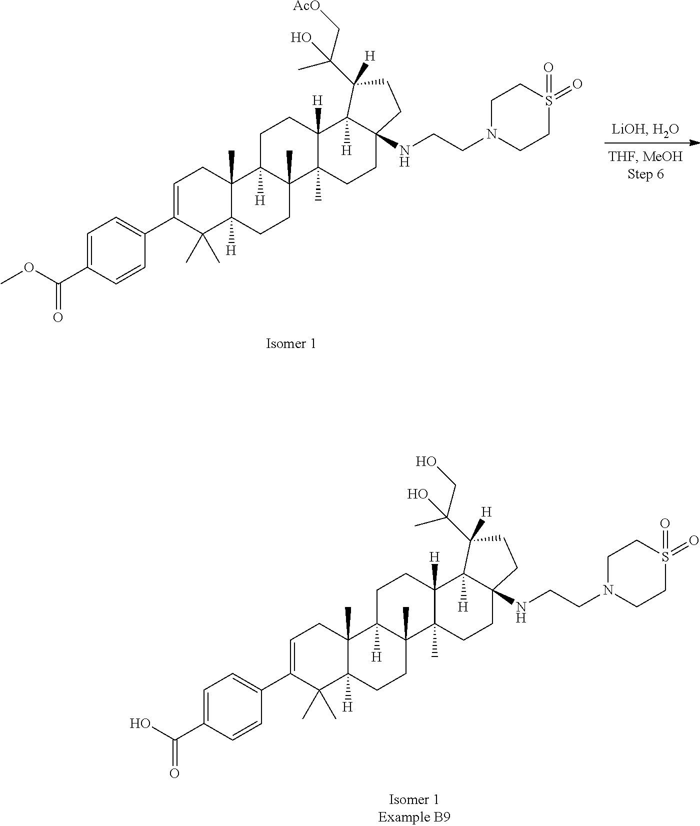

### Step 1. Preparation of methyl 4-((1R,3aS,5aR,5bR,7aR,11aS,11bR,13aR,13bR)-3a-((((9H-fluoren-9-yl)methoxy)carbonyl)amino)-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysen-9-yl)benzoate

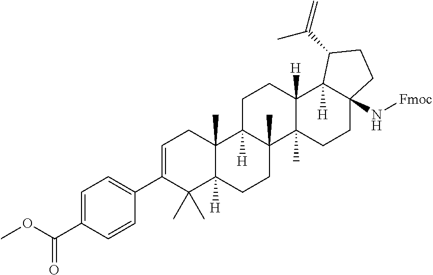

To a flask containing Intermediate 3 (4.00 g, 6.89 mmol) was added sodium bicarbonate (2.90 g, 34.5 mmol). The mixture was diluted with THF (80 mL) and water (25 mL), then 9-fluorenylmethoxycarbonyl-chloride (2.140 g, 8.27 mmol) was added, and the mixture was stirred at rt. After 2 h of stirring, TLC showed the reaction was complete. The mixture was diluted with 350 mL of EtOAc and was washed with water (3×100 mL). The organic was dried over MgSO₄, filtered and concentrated in vacuo. The residue (approx 5.5 g off white glassy solid) was purified by silica gel chromatography (elution gradient 96:4 hexanes:EtOAc to 65:35 hexanes:EtOAc over 10 column volumes) to give the product as a white solid. Total recovery=4.25 g (80% yield). ¹H NMR (400 MHz, CHLOROFORM-d) δ 7.96 (d, J=8.3 Hz, 2H), 7.80 (d, J=7.6 Hz, 2H), 7.64 (t, J=6.8 Hz, 2H), 7.44 (td, J=7.2, 4.0 Hz, 2H), 7.35 (tdd, J=7.5, 2.9, 1.0 Hz, 2H), 7.23 (d, J=8.3 Hz, 2H), 5.32 (d, J=4.6 Hz, 1H), 4.76 (br. s., 1H), 4.65 (br. s., 1H), 4.63-4.54 (m, 2H), 4.36-4.22 (m, 2H), 3.94 (s, 3H), 2.61-2.34 (m, 3H), 2.13 (dd, J=17.1, 6.4 Hz, 1H), 2.04-1.85 (m, 1H), 1.81-1.60 (m, 7H), 1.61-1.40 (m, 8H), 1.39-1.20 (m, 6H), 1.18-1.06 (m, 4H), 1.03 (s, 3H), 1.01 (br. s., 3H), 0.98 (s, 3H), 0.96 (s, 3H).

### Step 2. Preparation of methyl 4-((1R,3aS,5aR,5bR,7aR,11aS,11bR,13aR,13bS)-3a-((((9H-fluoren-9-yl)methoxy)carbonyl)amino)-1-(1,2-dihydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 1 and methyl 4-((1R,3aS,5aR,5bR,7aR,11aS,11bR,13aR,13bS)-3a-((((9H-fluoren-9-yl)methoxy)carbonyl)amino)-1-(1,2-dihydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 2.

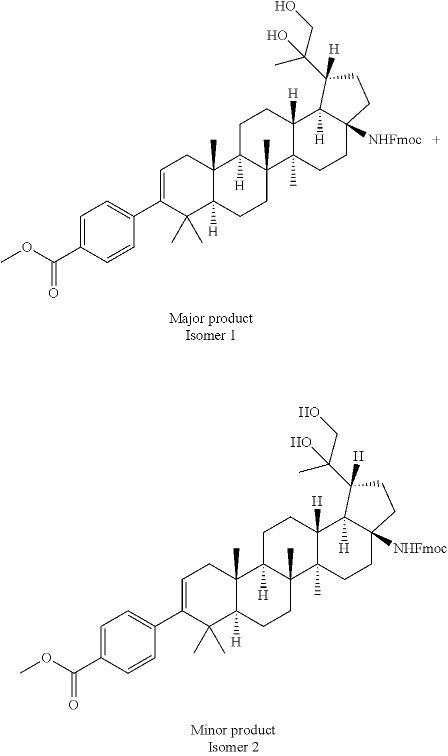

In a 20 mL scintillation vial with PTFE screwcap and stirbar were combined methyl 4-((1R,3aS,5aR,5bR,7aR,11aS,11bR,13aR,13bR)-3a-((((9H-fluoren-9-yl)methoxy)carbonyl)amino)-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysen-9-yl)benzoate (2.50 g, 3.26 mmol) with NMO (0.765 g, 6.53 mmol) in THF (60 mL) and water (10 mL). Then solid osmium tetroxide (0.415 g, 1.632 mmol) was introduced. The mixture was flushed with nitrogen, sealed and stirred at rt for 7 days. The yellow mixture was diluted with ethyl acetate (700 mL) and water (300 mL) and shaken and phases were separated. The organic was washed again with water (2×250 mL) and then with brine (50 mL). The organic was dried over sodium sulfate, filtered and concentrated in vacuo to a brown solid. The crude solid was purified by silica chromatography (elution gradient 100% hexanes to 40% EtOAc in hexanes, hold 40% EtOAc in hexanes for 4 column volumes, then gradient to 50% EtOAc in hexanes). Two products were isolated. The minor product (Isomer 2) was the first of the two isomers to elute from the column. Isomer 2 was isolated as a grey solid (0.183 g, 7.0% yield). ¹H NMR (400 MHz, CHLOROFORM-d) δ 7.96 (d, J=8.3 Hz, 2H), 7.80 (d, J=7.6 Hz, 2H), 7.64 (t, J=6.8 Hz, 2H), 7.47-7.39 (m, 2H), 7.38-7.31 (m, 2H), 7.23 (d, J=8.3 Hz, 2H), 5.33 (d, J=4.6 Hz, 1H), 4.67 (br. s., 1H), 4.55 (dd, J=10.3, 6.8 Hz, 1H), 4.37-4.28 (m, 1H), 4.28-4.21 (m, 1H), 3.94 (s, 3H), 3.58-3.41 (m, 2H), 2.60 (d, J=13.7 Hz, 1H), 2.40-2.28 (m, 1H), 2.21-2.11 (m, 1H), 2.10-2.06 (m, 1H), 2.02-1.89 (m, 3H), 1.88-1.68 (m, 4H), 1.69-1.31 (m, 12H), 1.26-1.16 (m, 2H), 1.11 (br. s., 6H), 1.04 (s, 6H), 0.98 (s, 3H), 0.96 (s, 3H). The major product (Isomer 1) was the second of the two isomers to elute from the silica column. Isomer 1 was isolated as a grey solid (1.165 g, 44.6% yield). ¹H NMR (400 MHz, CHLOROFORM-d) δ 8.00-7.93 (m, 2H), 7.80 (d, J=7.6 Hz, 2H), 7.64 (t, J=6.7 Hz, 2H), 7.48-7.39 (m, 2H), 7.38-7.31 (m, 2H), 7.23 (d, J=8.3 Hz, 2H), 5.39-5.29 (m, 1H), 4.65 (br. s., 1H), 4.55 (dd, J=10.5, 6.8 Hz, 1H), 4.43-4.29 (m, 1H), 4.28-4.22 (m, 1H), 3.94 (s, 3H), 3.66 (d, J=10.8 Hz, 1H), 3.46 (d, J=9.3 Hz, 1H), 2.59 (d, J=10.5Hz, 1H), 2.37-2.26 (m, 1H), 2.16 (dd, J=17.1, 6.4 Hz, 1H), 2.03-1.77 (m, 6H), 1.76-1.31 (m, 13H), 1.24 (s, 3H), 1.10 (br. s., 4H), 1.04 (s, 3H), 1.01 (br. s., 3H), 0.98 (s, 3H), 0.96 (s, 3H).

### Step 3. Preparation of methyl 4-((1R,3aS,5aR,5bR,7aR,11aS,11bR,13aR,13bS)-3a-((((9H-fluoren-9-yl)methoxy)carbonyl)amino)-1-(1-acetoxy-2-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 1

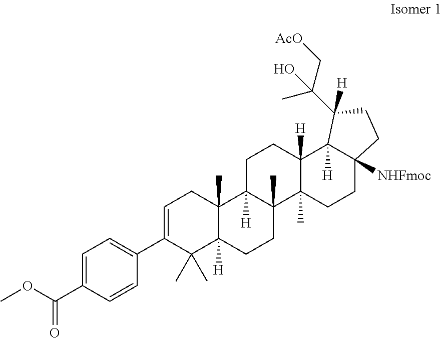

In a 20 mL scintillation vial with PTFE screwcap was dissolved methyl 4-((1R,3aS,5aR,5bR,7aR,11aS,11bR,13aR,13bS)-3a-((((9H-fluoren-9-yl)methoxy)carbonyl)amino)-1-(1,2-dihydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 1 (0.30 g, 0.375 mmol) in chloroform (5 mL). To the mixture was added pyridine (0.243 mL, 3.00 mmol), followed by addition of acetyl chloride (0.133 mL, 1.875 mmol). An exotherm was noticed. TLC confirmed that the reaction was complete within 5 min. The crude mixture was dissolved in minimum CHCl₃ and purified by silica gel chromatography (elution gradient 100% hexanes to 50% EtOAc in hexanes over 6 column volumes, hold 50% EtOAc in hexanes for 6 column volumes). The major product was thus isolated as a white solid. Total recovery=0.260 g (82% yield). ¹H NMR (400 MHz, CHLOROFORM-d) δ 7.96 (d, J=8.3 Hz, 2H), 7.80 (d, J=7.6 Hz, 2H), 7.64 (t, J=6.8 Hz, 2H), 7.47-7.39 (m, 2H), 7.37-7.30 (m, 2H), 7.23 (d, J=8.3 Hz, 2H), 5.33 (d, J=4.6 Hz, 1H), 4.63 (br. s., 1H), 4.57 (dd, J=10.3, 6.8 Hz, 1H), 4.35 (br. s., 1H), 4.28-4.21 (m, 1H), 4.11-4.02 (m, 2H), 3.94 (s, 3H), 2.58 (d, J=11.2 Hz, 1H), 2.38-2.27 (m, 1H), 2.20-2.09 (m, 4H), 1.98 (d, J=6.4 Hz, 1H), 1.93-1.82 (m, 3H), 1.82-1.32 (m, 14H), 1.26 (d, J=5.4 Hz, 4H), 1.09 (br. s., 4H), 1.03 (d, J=4.2Hz, 6H), 0.98 (s, 3H), 0.96 (s, 3H).

### Step 4. Preparation of methyl 4-((1R,3aS,5aR,5bR,7aR,11aS,11bR,13aR,13bS)-1-(1-acetoxy-2-hydroxypropan-2-yl)-3a-amino-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 1

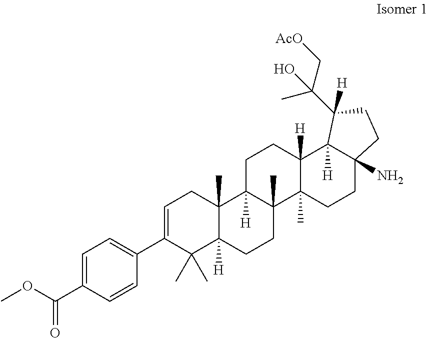

Methyl 4-((1R,3aS,5aR,5bR,7aR,11aS,11bR,13aR,13bS)-3a-((((9H-fluoren-9-yl)methoxy)carbonyl)amino)-1-(1-acetoxy-2-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 1 (0.260 g, 0.309 mmol) was dissolved in chloroform (5 mL) and piperidine (0.5 mL, 5.05 mmol) was added. The mixture was stirred at rt for 18 h. The reaction was concentrated in vacuo and the crude residue was purified by silica gel chromatography (elution gradient 100% DCM to 9:1 DCM:MeOH over 6 column volumes, hold 9:1 DCM:MeOH for 6 column volumes. Like product fractions were combined and concentrated in vacuo to give 0.1809 g (95% yield) of a slightly yellow foamy solid. ¹H NMR (400 MHz, CHLOROFORM-d) δ 7.95 (d, J=8.3 Hz, 2H), 7.23 (d, J=8.3 Hz, 2H), 5.32 (dd, J=6.2, 1.8 Hz, 1H), 4.14-4.01 (m, 2H), 3.93 (s, 3H), 2.14 (s, 6H), 1.99-1.76 (m, 4H), 1.76-1.44 (m, 11H), 1.43-1.17 (m, 11H), 1.15 (s, 3H), 1.02 (s, 6H), 0.96 (s, 6H).

### Step 5. Preparation of methyl 4-((1R,3aS,5aR,5bR,7aR,11aS,11bR,13aR,13bS)-1-(1-acetoxy-2-hydroxypropan-2-yl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 1.

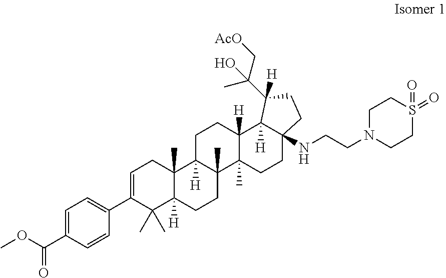

In a 75 mL medium pressure vessel containing methyl 4-((1R,3aS,5aR,5bR,7aR,11aS,11bR,13aR,13bS)-1-(1-acetoxy-2-hydroxypropan-2-yl)-3a-amino-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 1 (0.180 g, 0.290 mmol) was added 4-(2-chloroethyl)thiomorpholine 1,1-dioxide (0.150 g, 0.759 mmol), phosphoric acid, potassium salt (0.229 g, 1.079 mmol) and KI (0.110 g, 0.663 mmol). The mixture was diluted with acetonitrile (12 mL). The vessel was then flushed with N₂ and was sealed and heated to 110° C. in an oil bath for 16 h. The mixture was diluted with EtOAc (100 mL) and water (50 mL), shaken and phases were separated. The organic phase was dried over MgSO₄, filtered and concentrated in vacuo to a residue. The crude residue was purified by silica gel chromatography (elution gradient 100% DCM to 9:1 DCM:MeOH over 6 column volumes, hold 9:1 DCM:MeOH for 6 column volumes) to provide a yellow oil (0.297 g) which was carried directly into the next step without further purification. LCMS: m/z=781.7 (M+H)⁺, 2.21 min (method 5).

Step 6: In a 20 mL scintillation vial were combined crude methyl 4-((1R,3aS,5aR,5bR,7aR,11aS,11bR,13aR,13bS)-1-(1-acetoxy-2-hydroxypropan-2-yl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 1 (0.227 g, 0.290 mmol) and lithium hydroxide monohydrate (0.085 g, 2.030 mmol) with tetrahydrofuran (4 mL), MeOH (3 mL) and water (2 mL). The vial was capped with a PTFE screwcap and the mixture was heated to 75° C. with stirring for 2 h. Additional lithium hydroxide monohydrate (36 mg, 0.857 mmol) was added and the mixture was reheated to 75° C. for another 45 min. The mixture was purified by reverse phase preparative HPLC to afford 4-((1R,3aS,5aR,5bR,7aR,11aS,11bR,13aR,13bS)-1-(1,2-dihydroxypropan-2-yl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid, Isomer 1 (0.173 g, 61.2% yield) as a white powder TFA salt. LCMS: m/z=725.6 (M+H)⁺, 1.94 min (method 5). ¹H NMR (400 MHz, 1:1 mixture of chloroform-d and methanol-d₄, methanol-d₄ lock) δ 7.93 (d, J=8.3 Hz, 2H), 7.21 (d, J=8.3 Hz, 2H), 5.31 (d, J=4.6 Hz, 1H), 3.68-3.57 (m, 2H), 3.48-3.39 (m, 2H), 3.31-3.14 (m, 6H), 3.08 (br. s., 6H), 2.26 (d, J=8.8 Hz, 2H), 2.18 (dd, J=17.1, 6.4 Hz, 1H), 2.09-1.96 (m, 2H), 1.91-1.67 (m, 6H), 1.66-1.36 (m, 11H), 1.28 (d, J=10.5Hz, 1H), 1.22 (s, 3H), 1.21-1.16 (m, 6H), 1.14 (s, 3H), 1.04 (s, 3H), 0.96 (br. s., 3H), 0.95 (br. s., 3H).

### Example B10

### Preparation of 4-((1R,3aS,5aR,5bR,7aR,11aS,11bR,13aR,13bS)-1-(1,2-dihydroxypropan-2-yl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid, Isomer 2.

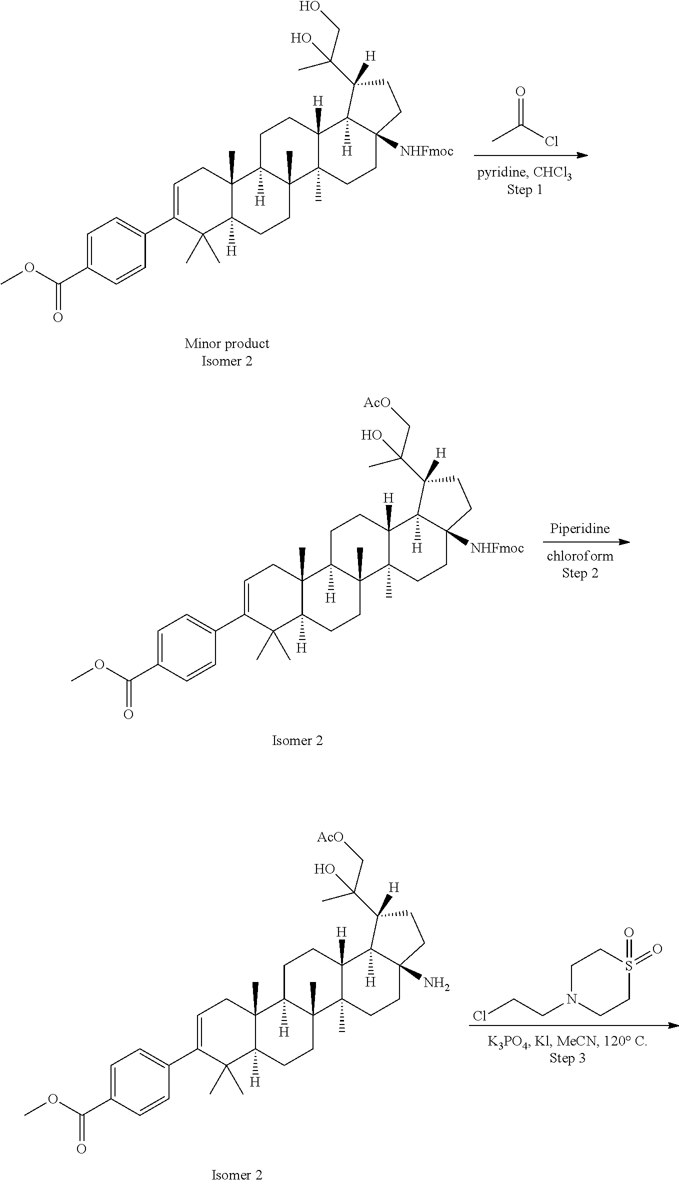

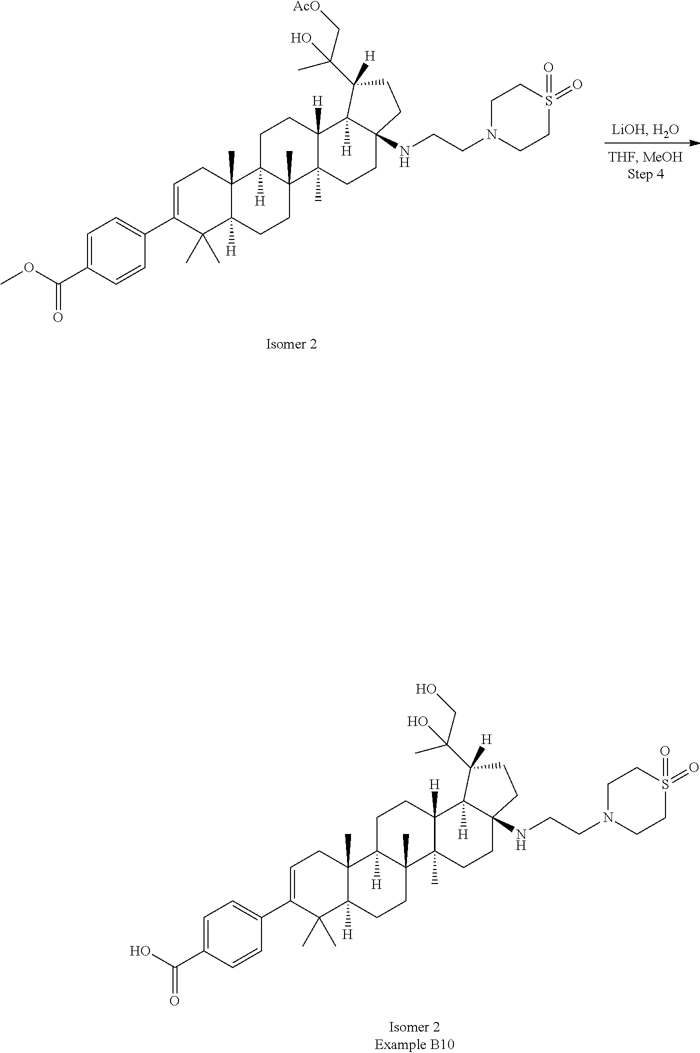

### Step 1. Preparation of methyl 4-((1R,3aS,5aR,5bR,7aR,11aS,11bR,13aR,13bS)-3a-((((9H-fluoren-9-yl)methoxy)carbonyl)amino)-1-(1-acetoxy-2-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 2.

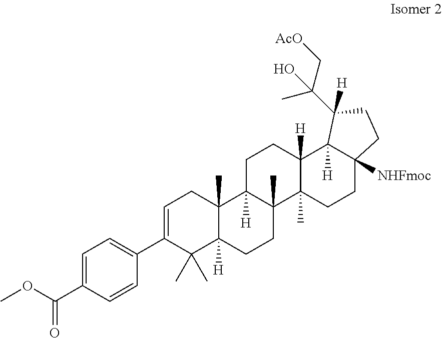

In a 20 mL scintillation vial was dissolved methyl 4-((1R,3aS,5aR,5bR,7aR,11aS,11bR,13aR,13bS)-3a-((((9H-fluoren-9-yl)methoxy)carbonyl)amino)-1-(1,2-dihydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 2 (0.169, 0.211 mmol) in chloroform (5 mL). To the mixture was added pyridine (0.137 mL, 1.690 mmol). The mixture was chilled in an ice bath and acetyl chloride (0.075 mL, 1.056 mmol) was added slowly. A PTFE screwcap was affixed to the vial and the mixture was stirred at rt for 10 min. The mixture was then concentrated in vacuo. The crude mixture was purified by silica gel chromatography (elution gradient 100% hexanes to 40% EtOAc in hexanes over 10 column volumes, hold 40% EtOAc in hexanes for 10 column volumes) to afford a slightly yellow solid, 0.1386 g (78% yield). ¹H NMR (400 MHz, CHLOROFORM-d) δ 7.96 (d, J=8.3 Hz, 2H), 7.80 (d, J=7.3 Hz, 2H), 7.64 (t, J=6.7 Hz, 2H), 7.47-7.40 (m, 2H), 7.38-7.32 (m, 2H), 7.23 (d, J=8.3 Hz, 2H), 5.33 (d, J=4.9 Hz, 1H), 4.66-4.56 (m, 2H), 4.39-4.29 (m, 1H), 4.28-4.22 (m, 1H), 4.10-3.99 (m, 2H), 3.94 (s, 3H), 2.58 (d, J=11.5Hz, 1H), 2.38-2.28 (m, 1H), 2.21-2.11 (m, 4H), 2.08-1.94 (m, 1H), 1.90 (s, 1H), 1.86-1.64 (m, 5H), 1.63-1.17 (m, 14H), 1.13 (s, 3H), 1.09 (br. s., 3H), 1.04 (s, 6H), 0.98 (s, 3H), 0.96 (s, 3H).

### Step 2. Preparation of methyl 4-((1R,3aS,5aR,5bR,7aR,11aS,11bR,13aR,13bS)-1-(1-acetoxy-2-hydroxypropan-2-yl)-3a-amino-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 2.

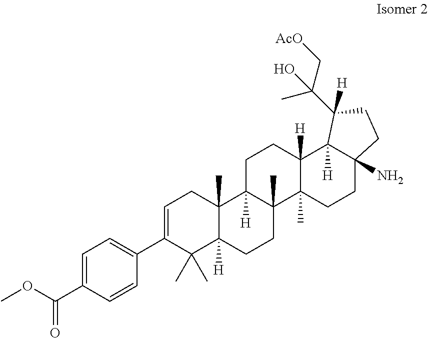

Methyl 4-((1R,3aS,5aR,5bR,7aR,11aS,11bR,13aR,13bS)-3a-((((9H-fluoren-9-yl)methoxy)carbonyl)amino)-1-(1-acetoxy-2-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 2 (0.135 g, 0.160 mmol) was dissolved in chloroform (5 mL) and piperidine (0.5 mL, 5.05 mmol) was added. The mixture was stirred at rt for 18 h. The mixture was concentrated via nitrogen stream, then purified by silica gel chromatography (elution gradient 100% DCM to 9:1 DCM:MeOH over 8 column volumes, hold 9:1 DCM:MeOH for 6 column volumes) to give a slightly yellow solid (0.0848 g, 85% yield). ¹H NMR (400 MHz, CHLOROFORM-d) δ 7.95 (d, J=8.3 Hz, 2H), 7.22 (d, J=8.3 Hz, 2H), 5.32 (dd, J=6.1, 1.5Hz, 1H), 4.15-3.96 (m, 2H), 3.93 (s, 3H), 2.21-2.10 (m, 5H), 2.09-1.75 (m, 5H), 1.71 (d, J=16.9 Hz, 1H), 1.67-1.44 (m, 10H), 1.44-1.30 (m, 5H), 1.30-1.18 (m, 3H), 1.16 (s, 3H), 1.14 (s, 4H), 1.04 (s, 3H), 1.02 (s, 3H), 0.96 (s, 3H), 0.95 (s, 3H).

### Step 3. Preparation of methyl 4-((1R,3aS,5aR,5bR,7aR,11aS,11bR,13aR,13bS)-1-(1-acetoxy-2-hydroxypropan-2-yl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yObenzoate Isomer 2.

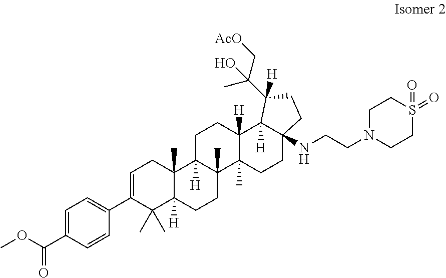

In a 15 mL medium pressure vessel were combined methyl 4-((1R,3aS,5aR,5bR,7aR,11aS,11bR,13aR,13bS)-1-(1-acetoxy-2-hydroxypropan-2-yl)-3a-amino-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 2 (0.082 g, 0.132 mmol), 4-(2-chloroethyl)thiomorpholine 1,1-dioxide (0.065 g, 0.331 mmol), phosphoric acid, potassium salt (0.098 g, 0.463 mmol) and KI (0.055 g, 0.331 mmol). The mixture was diluted with acetonitrile (6 mL). The vessel was then flushed with N₂ and was sealed and heated to 110° C. in an oil bath overnight. The mixture was diluted with chloroform (50 mL) and filtered to remove solids. The crude mixture was purified by silica gel chromatography (elution gradient 100% DCM to 9:1 DCM:MeOH over 6 column volumes, hold 9:1 DCM:MeOH for 6 column volumes) to give a very slightly yellow oil (0.099 g, 96% yield). LCMS: m/z=781.6 (M+H)⁺, 2.16 min (method 5). ¹H NMR (400 MHz, CHLOROFORM-d) δ 7.95 (d, J=8.3 Hz, 2H), 7.22 (d, J=8.3 Hz, 2H), 5.32 (dd, J=6.0, 1.6 Hz, 1H), 4.13-3.96 (m, 2H), 3.93 (s, 3H), 3.16-2.97 (m, 12H), 2.77-2.61 (m, 2H), 2.60-2.49 (m, 1H), 2.49-2.39 (m, 1H), 2.25-2.07 (m, 5H), 2.05-1.95 (m, 1H), 1.95-1.83 (m, 2H), 1.82 (s, 1H), 1.79-1.63 (m, 4H), 1.62-1.36 (m, 9H), 1.35-1.20 (m, 4H), 1.16 (s, 3H), 1.14 (s, 4H), 1.04 (s, 3H), 1.01 (s, 3H), 0.96 (s, 3H), 0.95 (br. s., 3H).

Step 4: In a 1 dram vial were combined methyl 4-((1R,3aS,5aR,5bR,7aR,11aS,11bR,13aR,13bS)-1-(1-acetoxy-2-hydroxypropan-2-yl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 2 (0.025 g, 0.032 mmol) and lithium hydroxide monohydrate (0.016 g, 0.384 mmol) with tetrahydrofuran (0.4 mL), MeOH (0.4 mL) and water (0.4 mL). The vial was capped with a PTFE screwcap and the mixture was heated to 75° C. with stirring for 2 h. The crude mixture was purified by reverse phase preparative HPLC (Prep HPLC Method 2) to give 4-((1R,3aS,5aR,5bR,7aR,11aS,11bR,13aR,13bS)-1-(1,2-dihydroxypropan-2-yl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid, Isomer 2 (0.026 g, 84% yield) as a white glassy solid TFA salt. LCMS: m/z=725.6 (M+H)⁺, 1.95 min (method 5). ¹H NMR (400 MHz, 1:1 mixture of chloroform-d and methanol-d₄, methanol-d₄ lock) δ 7.93 (d, J=8.3 Hz, 2H), 7.21 (d, J=8.3 Hz, 2H), 5.31 (d, J=4.4 Hz, 1H), 3.53 (d, J=11.2Hz, 1H), 3.32-3.23 (m, 5H), 3.23-3.14 (m, 2H), 3.13-3.00 (m, 6H), 2.40 (t, J=8.6 Hz, 1H), 2.23-2.03 (m, 3H), 1.98-1.90 (m, 1H), 1.89-1.77 (m, 3H), 1.73 (d, J=16.9 Hz, 1H), 1.69-1.36 (m, 12H), 1.32-1.24 (m, 2H), 1.21 (s, 3H), 1.13 (s, 3H), 1.09-1.05 (m, 3H), 1.05-1.01 (m, 3H), 0.98-0.96 (m, 3H), 0.95 (br. s., 3H).

### Examples B11 and B12

### Preparation of 4-((1R,3aS,5aR,5bR,7aR,11aS,11bR,13aR,13bS)-3a-((2-(1,1-dioxidothiomorpholino)ethyl)amino)-1-((R)-1-hydroxyethyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid Isomer 1 and 4-((1R,3aS,5aR,5bR,7aR,11aS,11bR,13aR,13bS)-3a-((2-(1,1-dioxidothiomorpholino)ethyl)amino)-1-((R)-1-hydroxyethyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid Isomer 2

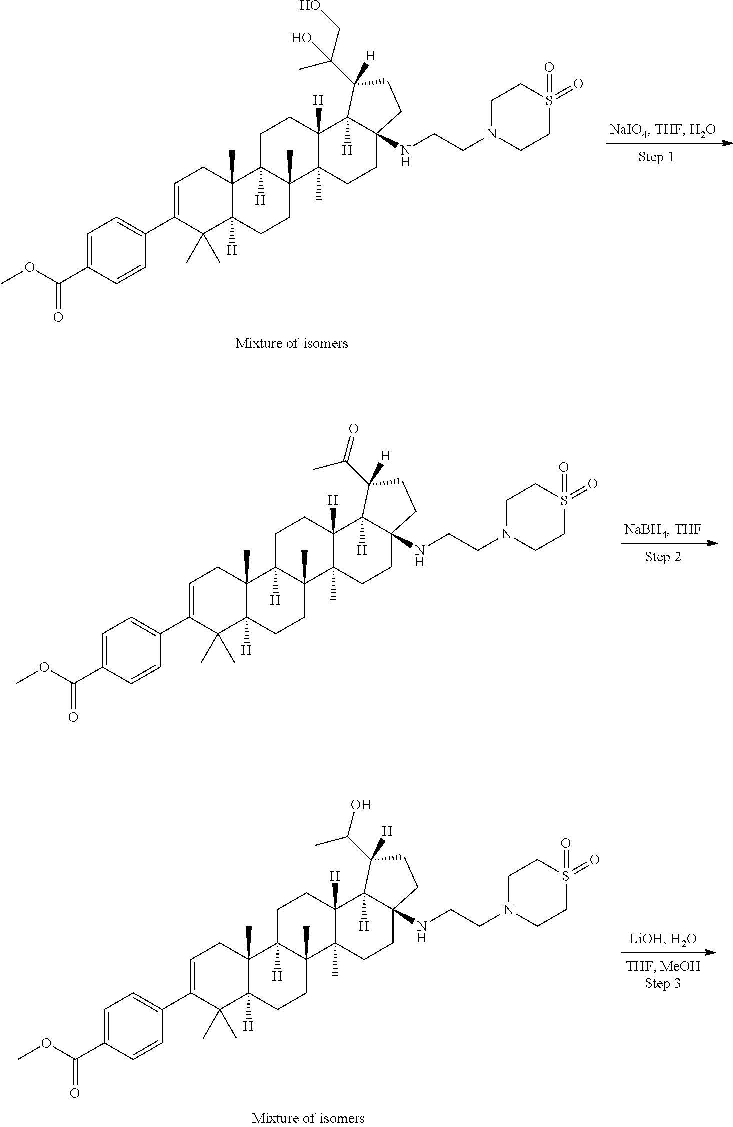

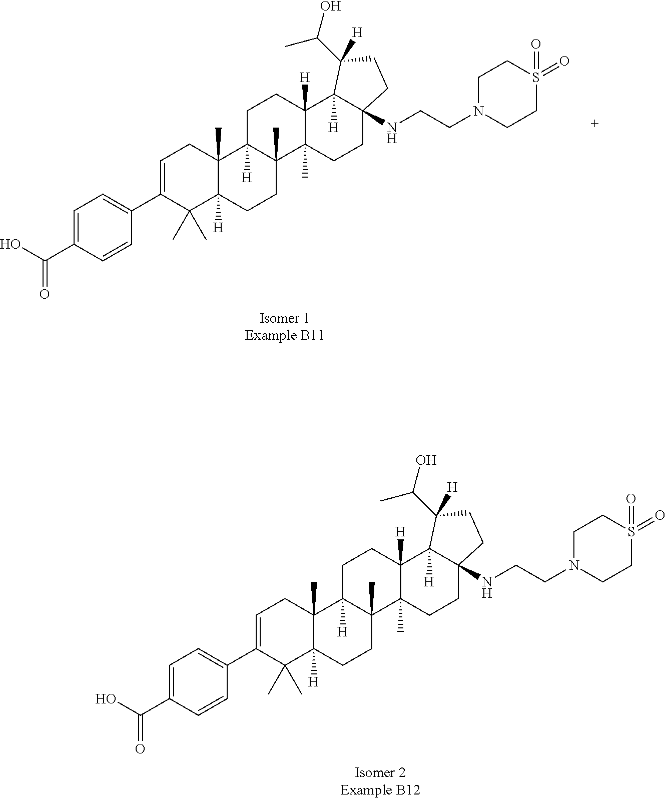

### Step 1. Preparation of methyl 4-((1R,3aS,5aR,5bR,7aR,11aS,11bR,13aR,13bS)-1-acetyl-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate

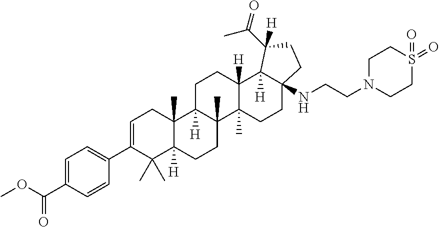

A mixture of both isomers of methyl 4-((1R,3aS,5aR,5bR,7aR,11aS,11bR,13aR,13bS)-1-(1,2-dihydroxypropan-2-yl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (0.280 g, 0.379 mmol) and sodium periodate (0.324 g, 1.515 mmol) was dissolved in a mixture of THF (10 mL) and water (2 mL). The mixture was stirred for 1 h and was then diluted with THF (75 mL) and brine (30 mL) and the resulting mixture was shaken and phases were separated. The aqueous was extracted with THF (2×50 mL) and then with chloroform (2×50 mL). The organics were combined and the cloudy result was concentrated to approximately 30 mL. Methanol was added until the organic was a complete solution, then silica gel (3 g) was added and the mixture was concentrated to a free-flowing powder which was placed in a vacuum oven overnight. The powder was loaded atop of a DCM pre-equilibrated 25 g silica gel cartridge. Elution (gradient 100% DCM to 40% of a 9:1 mixture of DCM:MeOH) gave the product (0.2462 g, 92% yield) as a slightly off-white glassy solid. LCMS: m/z=707.6 (M+H)⁺, 2.25 min (method 5). ¹H NMR (400 MHz, 1:1 mixture of chloroform-d and methanol-d₄, methanol-d₄ lock) δ 7.91 (d, J=8.3 Hz, 2H), 7.21 (d, J=8.3 Hz, 2H), 5.29 (d, J=4.9 Hz, 1H), 3.90 (s, 3H), 3.33 (s, 5H), 3.07-2.97 (m, 2H), 2.92 (td, J=11.0, 5.1 Hz, 1H), 2.76-2.66 (m, 2H), 2.66-2.57 (m, 1H), 2.48-2.39 (m, 1H), 2.20 (s, 3H), 2.17-2.01 (m, 3H), 1.94 (d, J=13.9 Hz, 1H), 1.87 (dd, J=12.7, 7.8 Hz, 1H), 1.75-1.63 (m, 2H), 1.62-1.41 (m, 8H), 1.39-1.18 (m, 5H), 1.15 (d, J=2.0 Hz, 1H), 1.12 (s, 4H), 1.08 (d, J=4.9 Hz, 1H), 1.04 (s, 3H), 1.00 (s, 3H), 0.94 (s, 3H), 0.93 (s, 3H).

### Step 2. Preparation of methyl 4-((1R,3aS,5aR,5bR,7aR,11aS,11bR,13aR,13bS)-3a-((2-(1,1-dioxidothiomorpholino)ethyl)amino)-1-(1-hydroxyethyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate.

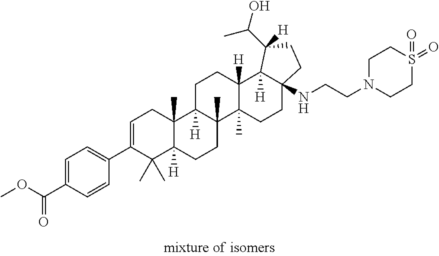

In a 1 dram vial with PTFE screwcap were combined methyl 4-((1R,3aS,5aR,5bR,7aR,11aS,11bR,13aR,13bS)-1-acetyl-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (0.020 g, 0.028 mmol) with sodium borohydride (0.00535 g, 0.141 mmol) in absolute ethanol (1 mL). The mixture was stirred at rt for 30 min, but the mixture was not fully dissolved. Added THF (1 mL) and the now complete solution was stirred at rt for 16 h. The crude mixture was partially purified by reverse phase preparative HPLC (Prep HPLC Method 4) to provide 0.026 g of TFA salt material as a mixture of isomers which was carried forward into the next step without further manipulation. LCMS: m/z=709.5 (M+H)⁺, 2.14 min (method 5).

Step 3: In a 1 dram vial with PTFE screwcap were combined the material from Step 2 containing the TFA salt of methyl 4-((1R,3aS,5aR,5bR,7aR,11aS,11bR,13aR,13bS)-3a-((2-(1,1-dioxidothiomorpholino)ethyl)amino)-1-((R)-1-hydroxyethyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (0.026 g, 0.014 mmol) with lithium hydroxide monohydrate, 1.0M aqueous solution (0.280 mL, 0.28 mmol) and Tetrahydrofuran (0.3 mL) and MeOH (0.3 mL). The vial was capped with PTFE screwcap and the mixture was heated to 70° C. with stirring for 45 min. The crude mixture was purified by reverse phase preparative HPLC (Prep HPLC Method 2). Two separate products were isolated. The first material to elute was the major product and was labeled 4-((1R,3aS,5aR,5bR,7aR,11aS,11bR,13aR,13b5)-3a-((2-(1,1-dioxidothiomorpholino)ethyl)amino)-1-(1-hydroxyethyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid Isomer 1 (0.0169 g, 61.5% yield white powder TFA salt). The minor product eluted second and was labeled 4-((1R,3aS,5aR,5bR,7aR,11aS,11bR,13aR,13b5)-3a-((2-(1,1-dioxidothiomorpholino)ethyl)amino)-1-(1-hydroxyethyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid Isomer 2 (0.0051 g, 18.6% yield white powder TFA salt).

Analytical data for 4-((1R,3aS,5aR,5bR,7aR,11aS,11bR,13aR,13b5)-3a-((2-(1,1-dioxidothiomorpholino)ethyl)amino)-1-(1-hydroxyethyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid Isomer 1: LCMS: m/z=695.4 (M+H)⁺, 1.95 min (method 5). ¹H NMR (400 MHz, 1:1 mixture of chloroform-d and methanol-d₄, methanol-d₄ lock) δ 7.93 (d, J=8.1 Hz, 2H), 7.21 (d, J=8.3 Hz, 2H), 5.31 (d, J=5.1 Hz, 1H), 3.88 (d, J=6.4 Hz, 2H), 3.27 (d, J=1.2Hz, 2H), 3.22-2.97 (m, 8H), 2.24-1.84 (m, 9H), 1.82-1.69 (m, 3H), 1.68-1.32 (m, 12H), 1.32-1.24 (m, 2H), 1.21 (s, 3H), 1.16 (d, J=6.4 Hz, 3H), 1.11 (s, 3H), 1.05 (s, 3H), 1.00-0.96 (m, 3H), 0.95 (br. s., 3H).

Analytical data for 4-((1R,3aS,5aR,5bR,7aR,11aS,11bR,13aR,13bS)-3a-((2-(1,1-dioxidothiomorpholino)ethyl)amino)-1-(1-hydroxyethyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid Isomer 2: LCMS: m/z=695.4 (M+H)⁺, 2.01 min (method 5). ¹H NMR (400 MHz, 1:1 mixture of chloroform-d and methanol-d₄, methanol-d₄ lock) δ 7.93 (d, J=8.3 Hz, 2H), 7.21 (d, J=8.3 Hz, 2H), 5.34-5.27 (m, 1H), 4.07 (dd, J=6.2, 4.5Hz, 1H), 3.31-2.99 (m, 11H), 2.60-2.48 (m, 1H), 2.17 (dd, J=16.8, 6.2Hz, 1H), 2.12-1.95 (m, 3H), 1.94-1.84 (m, 1H), 1.79-1.68 (m, 3H), 1.67-1.38 (m, 11H), 1.32-1.24 (m, 3H), 1.20 (s, 3H), 1.10 (s, 3H), 1.07-1.06 (m, 3H), 1.05 (s, 3H), 0.96 (br. s., 3H), 0.95 (br. s., 3H).

### Example B13

### Preparation of 4-((3aS,5aR,5bR,7aR,11aS,11bR,13aR,13bS)-3a-((2-(1,1-dioxidothiomorpholino)ethyl)amino)-1-ethylidene-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid.

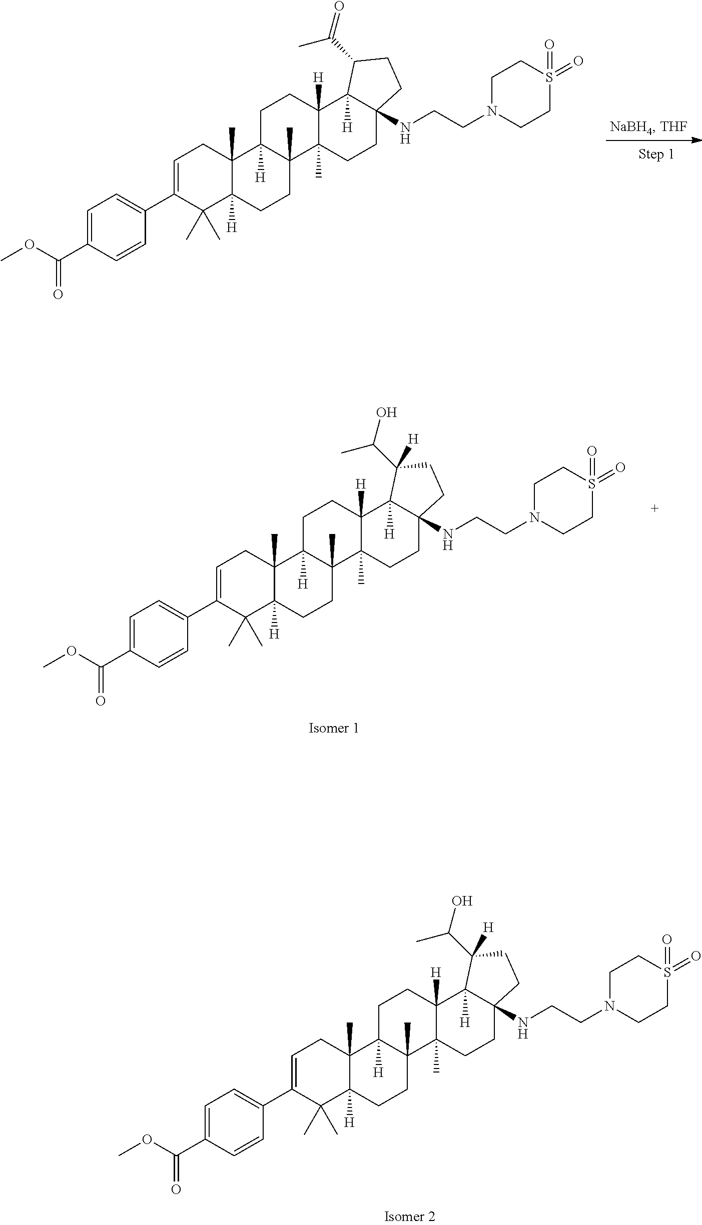

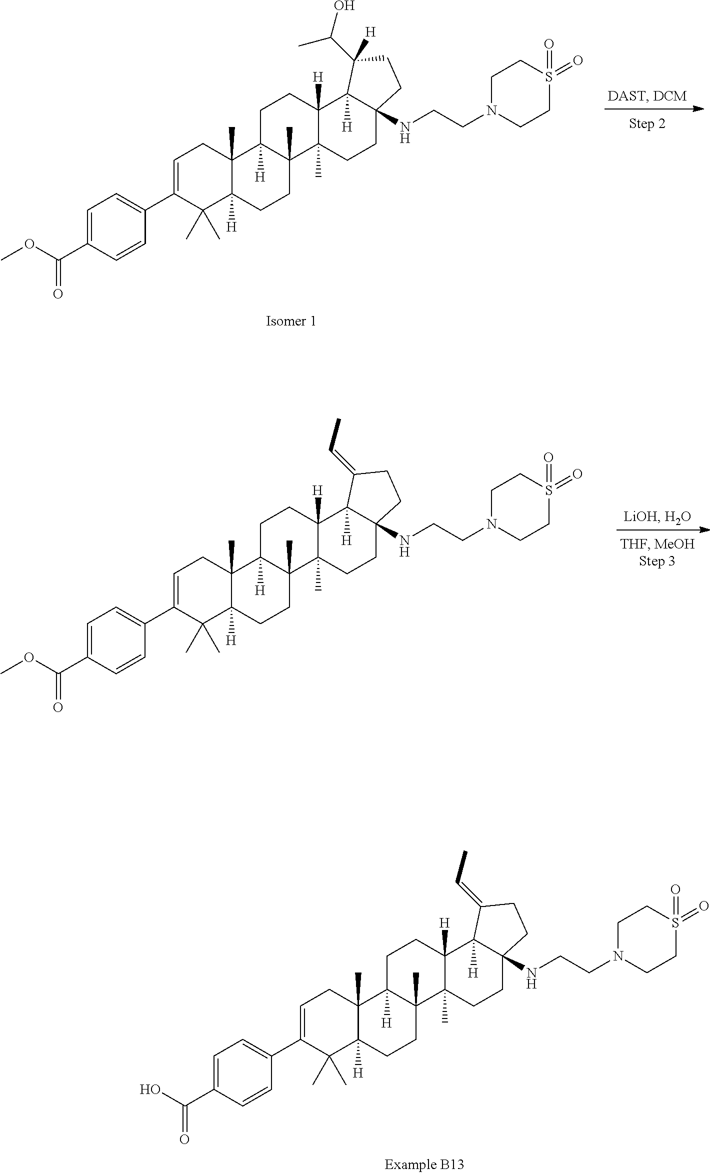

### Step 1. Preparation of methyl 4-((1R,3aS,5aR,5bR,7aR,11aS,11bR,13aR,13bS)-3a-((2-(1,1-dioxidothiomorpholino)ethyl)amino)-1-(1-hydroxyethyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 1 and methyl 4-((1R,3aS,5aR,5bR,7aR,11aS,11bR,13aR,13bS)-3a-((2-(1,1-dioxidothiomorpholino)ethyl)amino)-1-(1-hydroxyethyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 2.

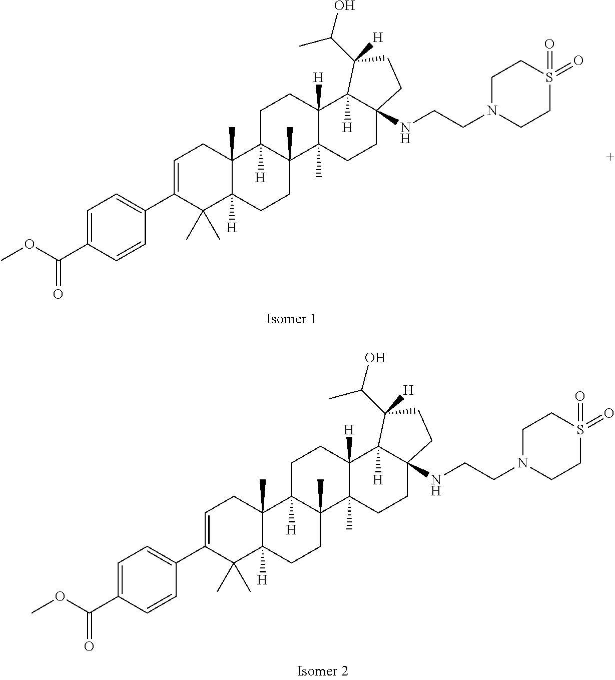

In a 7 mL scintillation vial with rubber septum under nitrogen atmosphere were combined methyl 4-((1R,3aS,5aR,5bR,7aR,11aS,11bR,13aR,13bS)-1-acetyl-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (0.100 g, 0.141 mmol) with sodium borohydride (0.021 g, 0.566 mmol) in a mixture of ethanol (2 mL) and dry THF (2 mL). The mixture was stirred at rt for 2 h. To the mixture was added saturated ammonium chloride dropwise until bubbling ceased. The mixture was diluted with a small amount of THF and filtered. The crude mixture was purified by reverse phase preparative HPLC (Prep HPLC Method 10). The first material to elute was the major product; this major product was labeled Isomer 1 and was isolated as a white powder (0.0547 g, 41.3% yield) TFA salt. The second material to elute was the minor product; this minor product was labeled Isomer 2 and was isolated as a white powder (0.0219 g, 16.5% yield) TFA salt. A portion of unreacted starting material (0.0328 g, 24.8%) was the third material to elute. Isomer 1 was dissolved in a 1:1 mixture of DCM and methanol and was loaded onto a 1 gram Waters Oasis MCX cation exchange cartridge. The cartridge was rinsed with 1:1 DCM:MeOH (20 mL) and then with MeOH (10 mL). The Isomer 1 material was then eluted from the cartridge with 2.0M ammonia in methanol (20 mL). Isomer 1 was thus recovered quantitatively as the free base material as a slightly off-white solid. Analytical data for methyl 4-((1R,3aS,5aR,5bR,7aR,11aS,11bR,13aR,13bS)-3a-((2-(1,1-dioxidothiomorpholino)ethyl)amino)-1-(1-hydroxyethyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 1: LCMS: m/z=709.4 (M+H)⁺, 2.18 min (method 5). ¹H NMR (400 MHz, 1:1 mixture of chloroform-d and methanol-d₄, methanol-d₄ lock) δ 7.91 (d, J=8.1 Hz, 2H), 7.21 (d, J=8.3 Hz, 2H), 5.30 (d, J=4.6 Hz, 1H), 3.95-3.83 (m, 4H), 3.20-2.98 (m, 8H), 2.88-2.46 (m, 4H), 2.15 (dd, J=17.1, 6.4 Hz, 1H), 1.95-1.83 (m, 3H), 1.83-1.63 (m, 5H), 1.63-1.41 (m, 9H), 1.41-1.22 (m, 4H), 1.20-1.10 (m, 7H), 1.04 (s, 3H), 1.02 (s, 3H), 0.95 (s, 3H), 0.94 (s, 3H).

### Step 2. Preparation of methyl 4-((3aS,5aR,5bR,7aR,11aS,11bR,13aR,13bS)-3a-((2-(1,1-dioxidothiomorpholino)ethyl)amino)-1-ethylidene-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate.

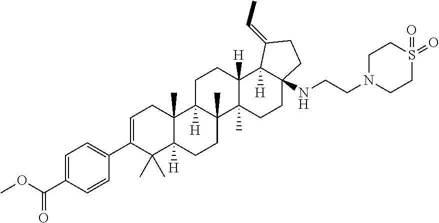

In a 1 dram vial with rubber stopper was placed the free base methyl 4-((1R,3aS,5aR,5bR,7aR,11aS,11bR,13aR,13bS)-3a-((2-(1,1-dioxidothiomorpholino)ethyl)amino)-1-((R)-1-hydroxyethyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 1 (0.025 g, 0.035 mmol) in DCM (1 mL). The mixture was chilled in a dry ice/acetone bath to −78 degrees C. and to the stirred mixture was added DAST (6.99 μl, 0.053 mmol). The cold bath was removed and the mixture was stirred at rt for 2 h. The mixture was concentrated under nitrogen stream to a residue which was redissolved in minimum THF:MeOH mixture (approx 4 to 1) and purified by reverse phase preparative HPLC (Prep HPLC Method 11). The product was thus isolated as a white solid (0.0139 g, 43% yield) TFA salt. LCMS: m/z=691.3 (M+H)⁺, 2.31 min (method 5). ¹H NMR (400 MHz, CHLOROFORM-d) δ 7.96 (d, J=8.3 Hz, 2H), 7.23 (d, J=8.6 Hz, 2H), 5.72-5.58 (m, 1H), 5.36-5.29 (m, 1H), 3.94 (s, 3H), 3.42-3.29 (m, 1H), 3.25-2.95 (m, 10H), 2.94-2.83 (m, 1H), 2.60-2.47 (m, 1H), 2.38-2.25 (m, 2H), 2.23-1.97 (m, 6H), 1.80 (br. s., 1H), 1.78-1.70 (m, 2H), 1.69-1.37 (m, 12H), 1.31-1.24 (m, 2H), 1.23-1.18 (m, 3H), 1.10 (s, 3H), 1.05 (s, 3H), 0.98 (s, 3H), 0.96 (s, 3H).

Step 3: In a 1 dram vial were combined methyl 4-((3aS,5aR,5bR,7aR,11aS,11bR,13aR,13bS)-3a-((2-(1,1-dioxidothiomorpholino)ethyl)amino)-1-ethylidene-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate, TFA salt (0.0139 g, 0.015 mmol) with lithium hydroxide monohydrate, 1.0M aqueous solution (0.151 mL, 0.151 mmol) and tetrahydrofuran (0.3 mL) and MeOH (0.3 mL). The vial was capped with a PTFE lined screwcap and the mixture was heated to 70 degrees C. for 35 min. The crude mixture was purified by reverse phase preparative HPLC (Prep HPLC method 4) to afford 4-((3aS,5aR,5bR,7aR,11aS,11bR,13aR,13bS)-3a-((2-(1,1-dioxidothiomorpholino)ethyl)amino)-1-ethylidene-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid as a white solid (0.0092 g, 61.8% yield) TFA salt. LCMS: m/z=677.4 (M+H)⁺, 2.17 min (method 5). ¹H NMR (400 MHz, Acetone) δ 7.98 (d, J=8.1 Hz, 2H), 7.30 (d, J=8.3 Hz, 2H), 5.68 (d, J=5.1 Hz, 1H), 5.35 (dd, J=6.4, 1.5 Hz, 1H), 3.26 (d, J=5.1 Hz, 9H), 2.63-2.44 (m, 3H), 2.43-2.29 (m, 2H), 2.25-2.14 (m, 4H), 1.88-1.72 (m, 4H), 1.70-1.51 (m, 10H), 1.24 (br. s., 8H), 1.14 (s, 3H), 1.09 (s, 3H), 1.01 (s, 3H), 0.99 (s, 3H).

### Example B14 and Example B15

### Preparation of 4-((1R,3aS,5aR,5bR,7aR,11aS,11bR,13aR,13bS)-3a-((2-(1,1-dioxidothiomorpholino)ethyl)amino)-1-(1-(hydroxyimino)ethyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid Isomer 1 and 4-((1R,3aS,5aR,5bR,7aR,11aS,11bR,13aR,13bS)-3a-((2-(1,1-dioxidothiomorpholino)ethyl)amino)-1-(1-(hydroxyimino)ethyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid Isomer 2.

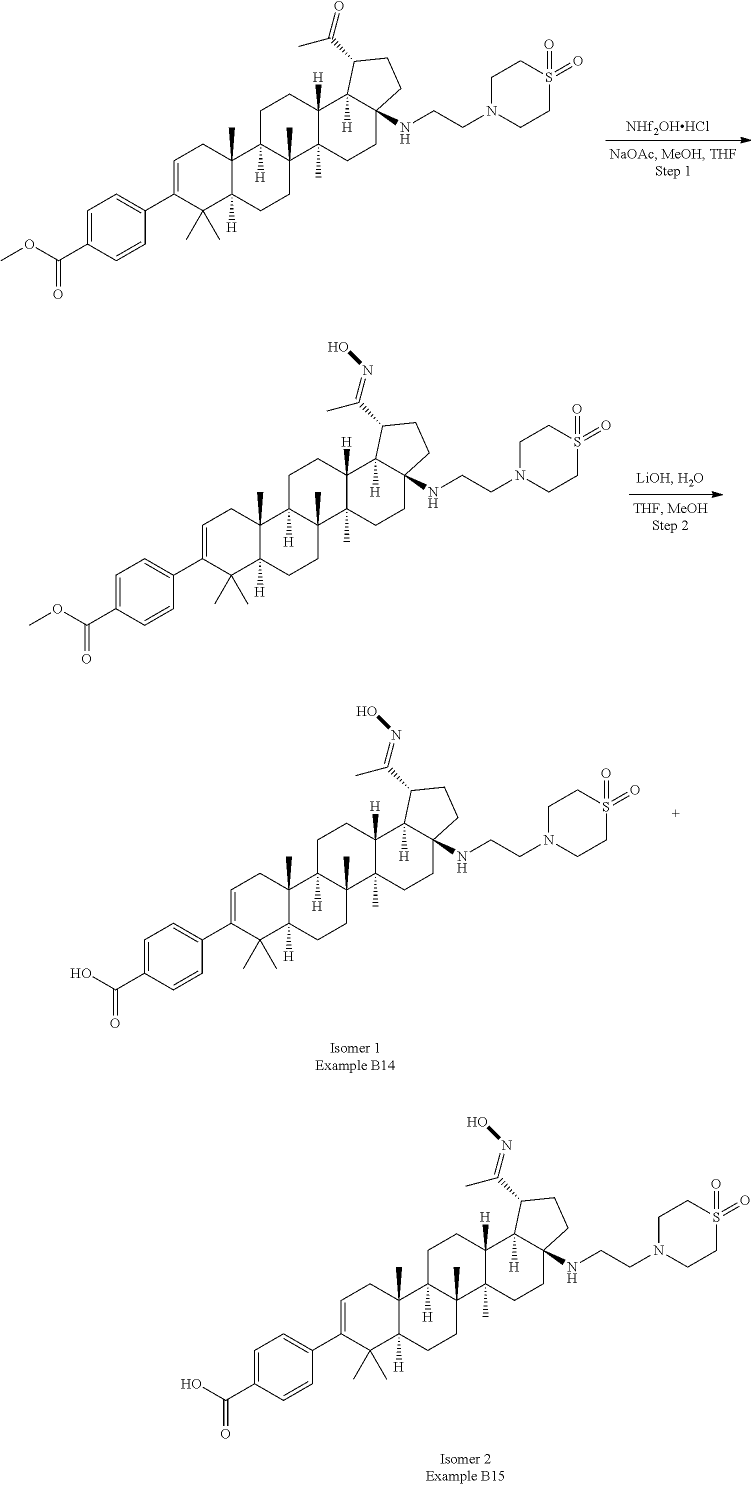

### Step 1. Preparation of methyl 4-((1R,3aS,5aR,5bR,7aR,11aS,11bR,13aR,13bS)-3a-((2-(1,1-dioxidothiomorpholino)ethyl)amino)-1-(1-(hydroxyimino)ethyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate

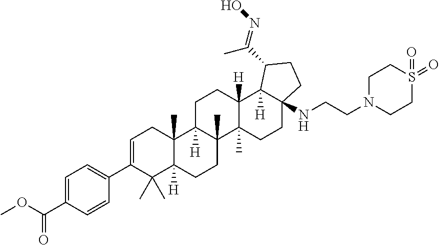

In a 1 dram vial with PTFE screwcap were combined methyl 4-((1R,3aS,5aR,5bR,7aR,11aS,11bR,13aR,13bS)-1-acetyl-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (0.028 g, 0.040 mmol) with hydroxylamine hydrochloride (0.028 g, 0.396 mmol) and sodium acetate (0.049 g, 0.594 mmol) in a mixture of dry methanol (2.3 mL) and tetrahydrofuran (1.5 mL). The suspension was stirred rapidly for 5 days and was then concentrated under nitrogen stream. The mixture was redissolved in a minimum amount of a mixture of THF and methanol and was purified by reverse phase preparative HPLC (prep HPLC method 12) The product was thus obtained as a white solid (0.0248 g, 87% yield). LCMS: m/z=722.4 (M+H)⁺, 2.15 min (method 5). ¹H NMR (400 MHz, CHLOROFORM-d) δ 7.95 (d, J=8.3 Hz, 2H), 7.22 (d, J=8.3 Hz, 2H), 5.31 (dd, J=6.0, 1.6 Hz, 1H), 3.93 (s, 3H), 3.18-2.97 (m, 8H), 2.86 (br. s., 1H), 2.76-2.55 (m, 3H), 2.49 (br. s., 1H), 2.13 (dd, J=17.2, 6.5Hz, 1H), 1.96-1.80 (m, 7H), 1.69 (d, J=17.1 Hz, 3H), 1.64-1.40 (m, 10H), 1.36 (td, J=12.7, 2.6 Hz, 2H), 1.29-1.20 (m, 3H), 1.12 (br. s., 4H), 1.01 (s, 6H), 0.96 (s, 3H), 0.95 (br. s., 3H).

Step 2: In a 1 dram vial were combined methyl 4-((1R,3aS,5aR,5bR,7aR,11aS,11bR,13aR,13bS)-3a-((2-(1,1-dioxidothiomorpholino)ethyl)amino)-1-((E)-1-(hydroxyimino)ethyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (0.020 g, 0.028 mmol) with lithium hydroxide monohydrate, 1.0M aqueous solution (0.277 mL, 0.277 mmol), tetrahydrofuran (0.3 mL) and MeOH (0.3 mL). The vial was capped with a PTFE lined screwcap and the mixture was heated to 70 degrees C. with stirring for 30 min. The crude mixture was purified by reverse phase preparative HPLC (Prep HPLC Method 12). Two peaks were collected. The first material to elute from the prep HPLC was labeled 4-((1R,3aS,5aR,5bR,7aR,11aS,11bR,13aR,13bS)-3a-((2-(1,1-dioxidothiomorpholino)ethyl)amino)-1-(1-(hydroxyimino)ethyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid Isomer 1 (0.0041 g, 20.7% yield) and was contaminated with the major Isomer 2 product. The second material to elute from the prep HPLC was the major product, labeled 4-((1R,3aS,5aR,5bR,7aR,11aS,11bR,13aR,13bS)-3a-((2-(1,1-dioxidothiomorpholino)ethyl)amino)-1-(1-(hydroxyimino)ethyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid Isomer 2 and isolated cleanly as a white powder (0.0119 g, 60.1% yield)

Analytical data for 4-((1R,3aS,5aR,5bR,7aR,11aS,11bR,13aR,13bS)-3a-((2-(1,1-dioxidothiomorpholino)ethyl)amino)-1-(1-(hydroxyimino)ethyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid, Isomer 1: HPLC showed that this material was a 73:27 mixture of Isomer 1 oxime to Isomer 2 oxime. LCMS: m/z=708.4 (M+H)⁺, 1.95 min (method 5). ¹H NMR (400 MHz, Acetone) δ 7.98 (d, J=8.1 Hz, 2H), 7.29 (d, J=8.1

Hz, 2H), 5.33 (dd, J=5.6, 1.2Hz, 1H), 3.94-3.78 (m, 1H), 3.19-3.01 (m, 11H), 2.25-2.13 (m, 1H), 2.12-2.09 (m, 2H), 2.02 (d, J=2.2Hz, 3H), 1.95-1.87 (m, 2H), 1.80 (s, 6H), 1.65-1.45 (m, 8H), 1.40-1.26 (m, 6H), 1.22 (s, 3H), 1.19-1.10 (m, 2H), 1.07 (d, J=1.5Hz, 6H), 1.00 (s, 3H), 0.98 (s, 3H). Analytical data for 4-((1R,3aS,5aR,5bR,7aR,11aS,11bR,13aR,13bS)-3a-((2-(1,1-dioxidothiomorpholino)ethyl)amino)-1-(1-(hydroxyimino)ethyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid, Isomer 2: HPLC showed that this material was a clean single compound. LCMS: m/z=708.4 (M+H)⁺, 1.99 min (method 5). ¹H NMR (400 MHz, Acetone) δ 7.98 (d, J=8.3 Hz, 2H), 7.29 (d, J=8.1 Hz, 2H), 5.33 (dd, J=6.0, 1.6 Hz, 1H), 3.16-2.98 (m, 10H), 2.85 (dt, J=10.8, 5.2Hz, 3H), 2.77-2.70 (m, 3H), 2.69-2.61 (m, 2H), 2.57-2.47 (m, 1H), 2.18 (dd, J=17.0, 6.5Hz, 1H), 1.98 (br. s., 1H), 1.96 (br. s., 1H), 1.93-1.84 (m, 1H), 1.79 (s, 4H), 1.77-1.71 (m, 2H), 1.67 (d, J=14.4 Hz, 1H), 1.63-1.57 (m, 2H), 1.47 (d, J=5.4 Hz, 4H), 1.45-1.27 (m, 5H), 1.27-1.22 (m, 1H), 1.21 (s, 3H), 1.10 (d, J=13.9 Hz, 1H), 1.06 (s, 3H), 1.05 (s, 3H), 0.99 (s, 3H), 0.98 (s, 3H).

### Example B16

### Preparation of 4-((1S,3aS,5aR,5bR,7aR,11aS,11bR,13aR,13bR)-3a-((2-(1,1-dioxidothiomorpholino)ethyl)amino)-5a,5b,8,8,11a-pentamethyl-1-(1-morpholinopropan-2-yl)-2,3,3a,4,5,5a,5b,6,7,7a,8,11,11a,11b,12,13,13a,13b-octadecahydro-1H-cyclopenta[a]chrysen-9-yl)benzoic acid.

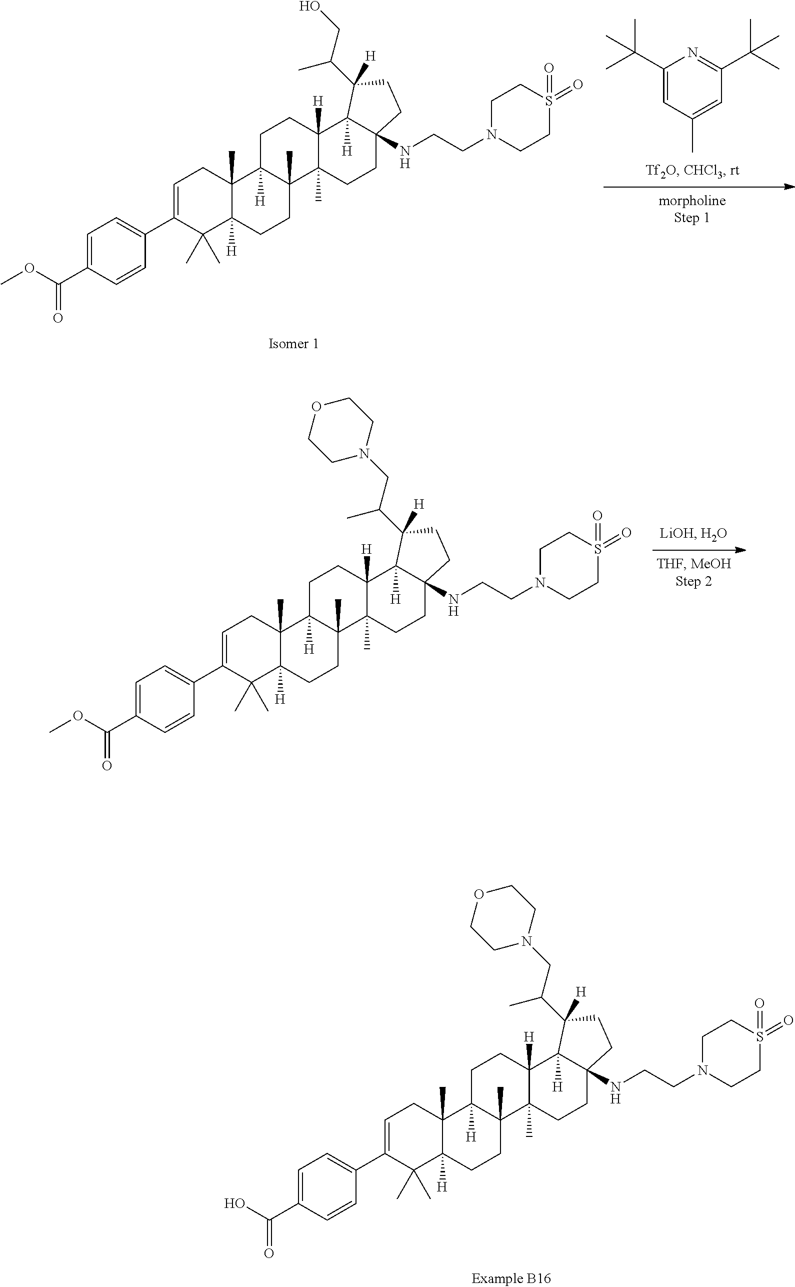

### Step 1. Preparation of methyl 4-((1S,3aS,5aR,5bR,7aR,11aS,11bR,13aR,13bR)-3a-((2-(1,1-dioxidothiomorpholino)ethyl)amino)-5a,5b,8,8,11a-pentamethyl-1-(1-morpholinopropan-2-yl)-2,3,3a,4,5,5a,5b,6,7,7a,8,11,11a,11b,12,13,13a,13b-octadecahydro-1H-cyclopenta[a]chrysen-9-yl)benzoate.

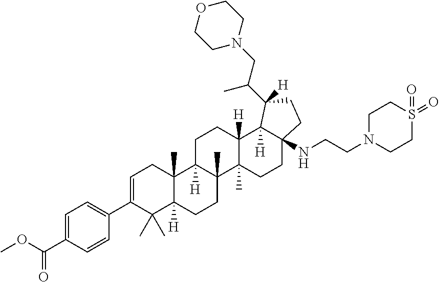

In a 1 dram vial with rubber septum and stirbar were combined methyl 4-((1R,3aS,5aR,5bR,7aR,11aS,11bR,13aR,13bR)-3a-((2-(1,1-dioxidothiomorpholino)ethyl)amino)-1-(1-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 1 (0.025 g, 0.035 mmol) and 2,6-di-tert-butyl-4-methylpyridine (0.021 g, 0.104 mmol) in dry chloroform (1 mL). To the stirred solution was added dropwise trifluoromethanesulfonic anhydride (0.013 g, 0.045 mmol) at rt. The mixture was stirred at rt for 1 h, then to the mixture was added morpholine (0.030 mL, 0.346 mmol) and the resulting solution was stirred at rt for 30 min and was then heated to 60 degrees C. for 90 min. The reaction mixture was concentrated under nitrogen stream and redissolved in a minimum quantity of a mixture of THF and MeOH. Purification by reverse phase preparative HPLC (Prep HPLC Method 3) gave the desired product as a white powder (0.0199 g, 50.7% yield) TFA salt. LCMS: m/z=792.7 (M+H)⁺, 1.97 min (method 5).

Step 2: In a 1 dram vial were combined methyl 4-((1S,3aS,5aR,5bR,7aR,11aS,11bR,13aR,13bR)-3a-((2-(1,1-dioxidothiomorpholino)ethyl)amino)-5a,5b,8,8,11a-pentamethyl-1-(1-morpholinopropan-2-yl)-2,3,3a,4,5,5a,5b,6,7,7a,8,11,11a,11b,12,13,13a,13b-octadecahydro-1H-cyclopenta[a]chrysen-9-yl)benzoate, TFA salt (0.0192 g, 0.019 mmol) with lithium hydroxide monohydrate, 1.0M aqueous solution (0.188 mL, 0.188 mmol) in tetrahydrofuran (0.3 mL) and MeOH (0.3 mL). The vial was capped with a PTFE lined screwcap and the mixture was heated with stirring to 70 degrees C. for 40 min. The crude mixture was purified by reverse phase preparative HPLC (Prep HPLC Method 3) to provide 4-((1S,3aS,5aR,5bR,7aR,11aS,11bR,13aR,13bR)-3a-((2-(1,1-dioxidothiomorpholino)ethyl)amino)-5a,5b,8,8,11a-pentamethyl-1-(1-morpholinopropan-2-yl)-2,3,3a,4,5,5a,5b,6,7,7a,8,11,11a,11b,12,13,13a,13b-octadecahydro-1H-cyclopenta[a]chrysen-9-yl)benzoic acid as a white powder TFA salt (0.0139 g, 73% yield). LCMS: m/z=778.7 (M+H)⁺, 1.75 min (method 5). ¹H NMR (400 MHz, 1:1 mixture of chloroform-d and methanol-d₄, methanol-d₄ lock) δ 7.93 (d, J=8.3 Hz, 2H), 7.22 (s, 2H), 5.31 (d, J=4.6 Hz, 1H), 3.96 (br. s., 4H), 3.31-2.95 (m, 16H), 2.38 (br. s., 1H), 2.17 (dd, J=17.1, 6.1 Hz, 2H), 2.07 (d, J=13.9 Hz, 1H), 2.03-1.89 (m, 3H), 1.89-1.82 (m, 1H), 1.81-1.72 (m, 2H), 1.72-1.40 (m, 12H), 1.39-1.25 (m, 3H), 1.22 (s, 3H), 1.15 (d, J=6.4 Hz, 3H), 1.12 (s, 3H), 1.05 (s, 3H), 0.97 (br. s., 3H), 0.96 (br. s., 3H).

### Example B17

### Preparation of (1R,3aS,5aR,5bR,7aR,11aS,11bR,13aR,13bS)-9-(4-carboxyphenyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-1-carboxylic acid

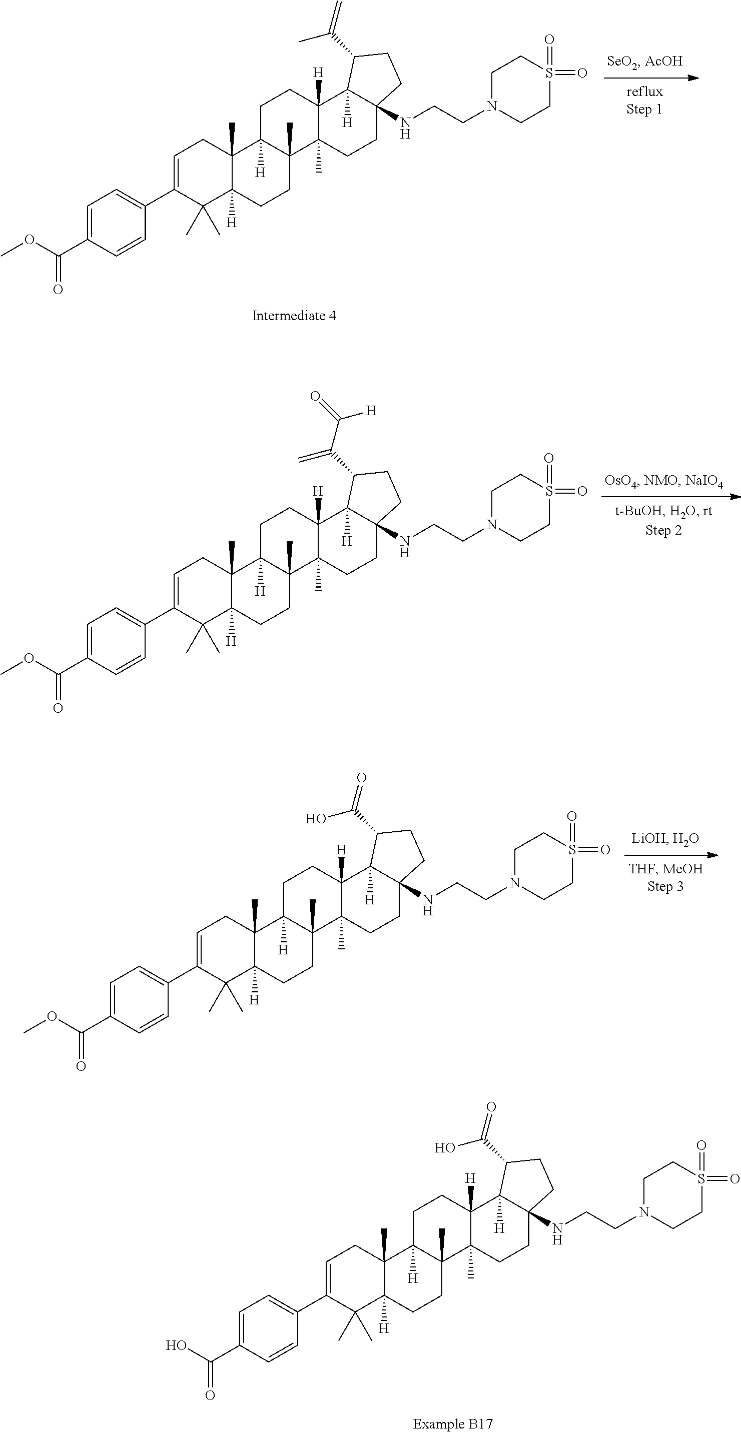

### Step 1. Preparation of methyl 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(3-oxoprop-1-en-2-yl)-2,3,3a,4,5,5a,5b,6,7,7a,8,11,11a,11b,12,13,13a,13b-octadecahydro-1H-cyclopenta[a]chrysen-9-yl)benzoate.

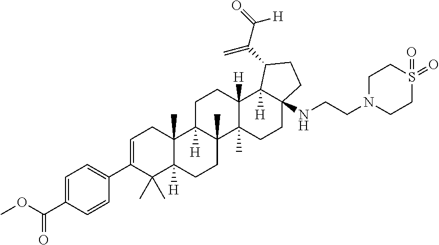

In a 75 mL Chemglass pressure vessel were combined Intermediate 4 methyl 4-((1R,3aS,5aR,5bR,7aR,11aS,11bR,13aR,13bR)-3a-((2-(1,1-dioxidothiomorpholino)ethyl)amino)-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysen-9-yl)benzoate (1.50 g, 2.13 mmol) with selenium dioxide (0.295 g, 2.66 mmol) and acetic acid (30 mL). The vessel was sealed and the mixture was heated to 100° C. for 45 min. The mixture was allowed to cool to rt and an additional 0.25 equivalents of selenium dioxide (0.059 g, 0.67 mmol) was added. The vessel was reheated to 100° C. for 15 min. The mixture was filtered through a fine frit funnel to remove a fine black solid, and the filtrate was concentrated in vacuo to an orange colored foamy residue. The crude mixture was purified by silica gel chromatography (elution gradient 100% hexanes to 3:1 hexanes:acetone over 6 column volumes, hold 3:1 hexanes:acetone for 6 column volumes). The product fractions were combined and concentrated to give the product as an off-white solid: 0.747 g (48.8% yield). LCMS: m/z=719.6 (M+H)⁺, 2.56 min (method 3).

### Step 2. Preparation of (1R,3aS,5aR,5bR,7aR,11aS,11bR,13aR,13bS)-3a-((2-(1,1-dioxidothiomorpholino)ethyl)amino)-9-(4-(methoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-1-carboxylic acid.

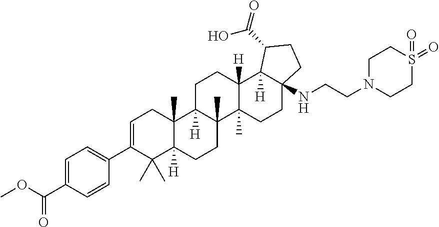

In a 100 mL round bottom flask sealed with rubber stopper were combined methyl 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(3-oxoprop-1-en-2-yl)-2,3,3a,4,5,5a,5b,6,7,7a,8,11,11a,11b,12,13,13a,13b-octadecahydro-1H-cyclopenta[a]chrysen-9-yl)benzoate (0.590 g, 0.821 mmol) with citric acid monohydrate (0.345 g, 1.64 mmol). Then, tert-butanol (18 mL) was added followed by water (15 mL). To the stirred mixture was added NMO, 50% by weight solution in water (0.374 mL, 1.81 mmol) followed by osmium tetroxide, 2.5% in t-butanol (0.515 mL, 0.041 mmol). The mixture was stirred at rt for 30.5 hours. To the mixture was added solid sodium periodate (1.229 g, 5.74 mmol). Solids began to precipitate from solution within 5 min. After stirring for 90 min, the reaction mixture was diluted with 125 mL of water, which caused a heavy fine precipitate to occur. The suspension was filtered to isolate a fine grayish solid which was purified by reverse phase preparative HPLC (Prep HPLC Method 13) to give an off-white powder: 0.2386 g (41.0% yield). LCMS: m/z=709.5 (M+H)⁺, 1.68 min (method 3). ¹H NMR (400 MHz, 1:1 mixture of chloroform-d and methanol-d₄, methanol-d₄ lock) δ 7.91 (d, J=8.3 Hz, 2H), 7.21 (d, J=8.3 Hz, 2H), 5.33-5.26 (m, 1H), 3.91 (s, 3H), 3.19-2.99 (m, 8H), 2.79-2.58 (m, 4H), 2.55-2.46 (m, 1H), 2.13 (dd, J=17.1, 6.4 Hz, 2H), 2.07-1.98 (m, 1H), 1.94 (d, J=12.7 Hz, 1H), 1.87 (dd, J=12.7, 7.8 Hz, 1H), 1.82-1.76 (m, 1H), 1.76-1.66 (m, 2H), 1.65-1.41 (m, 9H), 1.41-1.32 (m, 2H), 1.31-1.21 (m, 2H), 1.17 (br. s., 1H), 1.14 (s, 3H), 1.11-1.08 (m, 1H), 1.04 (s, 3H), 1.01 (s, 3H), 0.95 (s, 3H), 0.93 (s, 3H).

Step 3: In a 1 dram vial were combined (1R,3aS,5aR,5bR,7aR,11aS,11bR,13aR,13bS)-3a-((2-(1,1-dioxidothiomorpholino)ethyl)amino)-9-(4-(methoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-1-carboxylic acid (0.020 g, 0.028 mmol) with lithium hydroxide monohydrate, 1.0M aqueous solution (0.282 mL, 0.282 mmol), tetrahydrofuran (0.3 mL) and MeOH (0.3 mL). The vial was capped with a PTFE lined screwcap and the mixture was heated to 70° C. for 30 min. The crude mixture was purified by reverse phase preparative HPLC (Prep HPLC Method 2). Product fractions were combined and concentrated in vacuo to afford (1R,3aS,5aR,5bR,7aR,11aS,11bR,13aR,13b5)-9-(4-carboxyphenyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-1-carboxylic acid as a white powder TFA salt (0.0212 mg, 80% yield). LCMS: m/z=695.4 (M+H)⁺, 1.93 min (method 5).

### Example B18

### Preparation of 4-((1S,3aS,5aR,5bR,7aR,11aS,11bR,13aR,13bR)-3a-((2-(1,1-dioxidothiomorpholino)ethyl)amino)-1-(1-(1,1-dioxidothiomorpholino)propan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid.

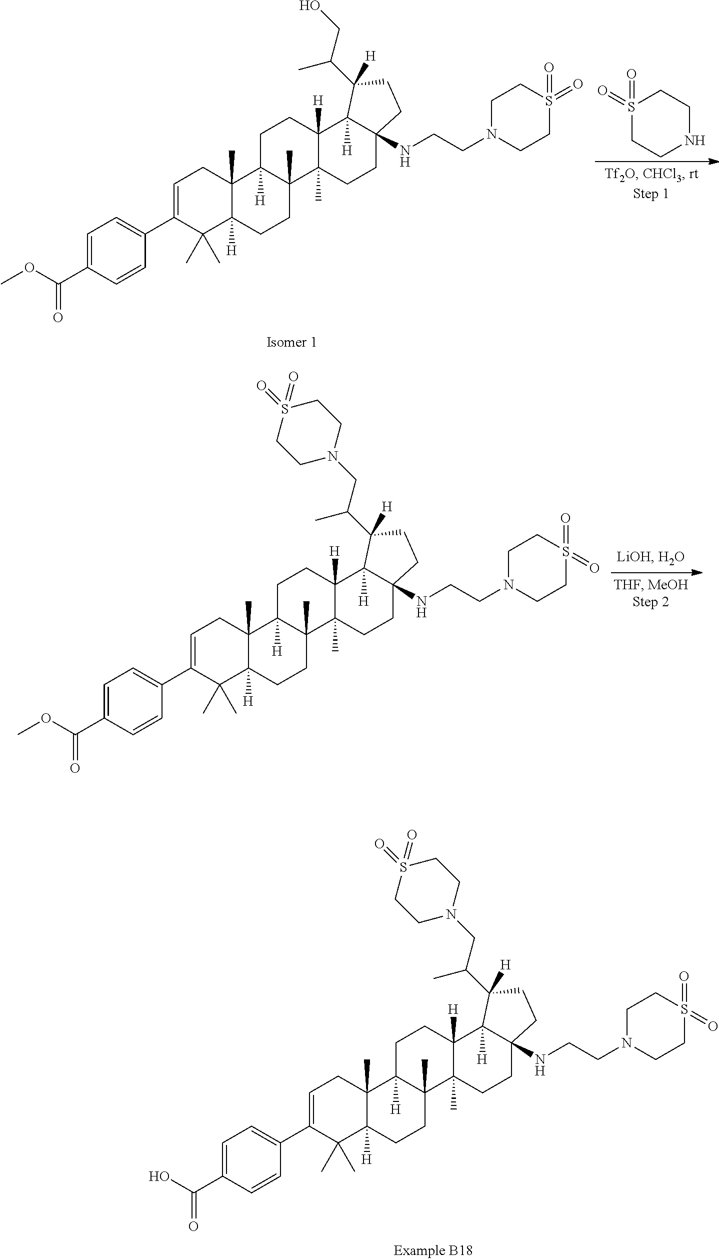

### Step 1. Preparation of methyl 4-((1S,3aS,5aR,5bR,7aR,11aS,11bR,13aR,13bR)-3a-((2-(1,1-dioxidothiomorpholino)ethyl)amino)-1-(1-(1,1-dioxidothiomorpholino)propan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate.

In a 1 dram vial with rubber septum and stirbar were combined methyl 4-((1R,3aS,5aR,5bR,7aR,11aS,11bR,13aR,13bR)-3a-((2-(1,1-dioxidothiomorpholino)ethyl)amino)-1-(1-hydroxypropan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 1 (0.150 g, 0.207 mmol) and dry chloroform (2 mL). The mixture was chilled in an ice/salt bath, and to the −10° C. stirred slurry was added trifluoromethanesulfonic anhydride (0.046 mL, 0.270 mmol). The resulting mixture was stirred at −10 C for 1 h, then to the mixture was added thiomorpholine 1,1-dioxide (0.280 g, 2.075 mmol) and the mixture was heated to 70 degrees C. with stirring. The crude mixture was concentrated and was redissolved in a minimum amount of THF and MeOH and purified by reverse phase preparative HPLC (Prep HPLC Method 2). The product was thus isolated as a white glassy solid TFA salt (0.0105 g, 4.3% yield). LCMS: m/z=840.6 (M+H)⁺, 2.16 min (method 5).

Step 2: In a 1 dram vial were combined methyl 4-((1S,3aS,5aR,5bR,7aR,11aS,11bR,13aR,13bR)-3a-((2-(1,1-dioxidothiomorpholino)ethyl)amino)-1-(1-(1,1-dioxidothiomorpholino)propan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate, TFA salt (0.0106 g, 9.92 mmol) with lithium hydroxide monohydrate, 1.0M aqueous solution (0.099 mL, 0.099 mmol) in tetrahydrofuran (0.5 mL) and MeOH (0.5 mL). The mixture was heated with stirring to 70 degrees C. for 60 min. The crude reaction mixture was concentrated via nitrogen stream, then redissolved in acetonitrile/methanol, filtered and purified by reverse phase preparative HPLC (Prep HPLC Method 2) to provide 4-((1S,3aS,5aR,5bR,7aR,11aS,11bR,13aR,13bR)-3a-((2-(1,1-dioxidothiomorpholino)ethyl)amino)-1-(1-(1,1-dioxidothiomorpholino)propan-2-yl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid as a white powder TFA salt (0.0080 g, 65.6% yield). LCMS: m/z=826.6 (M+H)⁺, 1.94 min (method 5). ¹H NMR (400 MHz, 1:1 mixture of chloroform-d and methanol-d₄, methanol-d₄ lock) δ 7.93 (d, J=8.3 Hz, 2H), 7.21 (d, J=8.3 Hz, 2H), 5.35-5.27 (m, 1H), 3.28 (d, J=1.5Hz, 4H), 3.23-3.02 (m, 13H), 3.01-2.93 (m, 2H), 2.55 (dd, J=13.3, 4.0 Hz, 1H), 2.36-2.24 (m, 2H), 2.17 (dd, J=17.2, 6.5Hz, 1H), 2.13-1.97 (m, 3H), 1.96-1.85 (m, 3H), 1.81-1.67 (m, 4H), 1.66-1.54 (m, 5H), 1.53-1.40 (m, 5H), 1.34-1.24 (m, 4H), 1.22 (s, 3H), 1.10 (s, 3H), 1.06 (s, 3H), 0.99 (d, J=6.8 Hz, 3H), 0.98-0.97 (m, 3H), 0.96 (br. s., 3H).

### Example B19

### Preparation of 4-((1R,3aS,5aR,5bR,7aR,11aS,11bR,13aR,13bS)-1-(1-aminoethyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid Isomer 1.

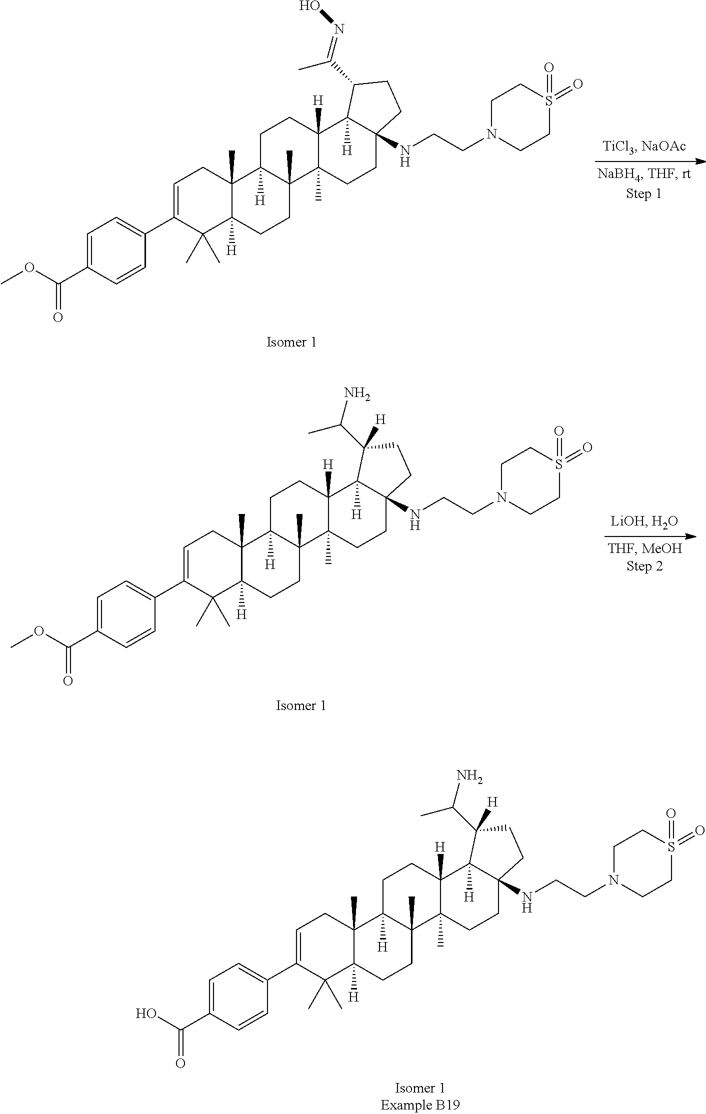

### Step 1. Preparation of methyl 4-((1R,3aS,5aR,5bR,7aR,11aS,11bR,13aR,13bS)-1-(1-aminoethyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 1.

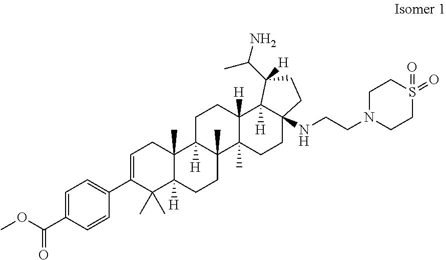

In a 20 mL scintillation vial were combined titanium (III) chloride, 20% wt solution in 3% HCl (0.766 mL, 1.21 mmol) and sodium acetate (0.099 g, 1.212 mmol) in ethanol (1 mL). The resulting lavender colored solution was cooled in an ice bath and THF (2 mL) was added followed by addition of methyl 4-((1R,3aS,5aR,5bR,7aR,11aS,11bR,13aR,13bS)-3a-((2-(1,1-dioxidothiomorpholino)ethyl)amino)-1-(1-(hydroxyimino)ethyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (0.175 g, 0.242 mmol). The vial was capped with a PTFE lined screwcap and the resulting suspension was stirred at rt for 30 min, then concentrated under nitrogen stream to a solid and placed under high vacuum at rt overnight. THF (2 mL) and ethanol (1 mL) were added, then solid sodium borohydride (0.092 g, 2.424 mmol) was added slowly, resulting in significant outgassing from the reaction mixture. At t=130 min, an excess of sodium borohydride was added (via spatula, approximately another 10 equivalents or more) significant outgassing occurred immediately and the mixture was stirred at rt for a total of 16 hours. To the reaction mixture was slowly added aqueous ammonium chloride to quench the reaction. The mixture was concentrated under nitrogen stream to a residue and was then redissolved in minimum acetonitrile/methanol/water, filtered and purified by reverse phase preparative HPLC (Prep HPLC Method 2) to provide the desired material as a single isomer beige solid TFA salt (0.079 g, 31% yield). LCMS: m/z=708.5 (M+H)⁺, 2.06 min (method 5).

Step 2: In a 1 dram vial were combined methyl 4-((1R,3aS,5aR,5bR,7aR,11aS,11bR,13aR,13bS)-1-((R)-1-aminoethyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 1 TFA salt (0.040 g, 0.038 mmol) with lithium hydroxide monohydrate, 1.0M aqueous solution (0.381 mL, 0.381 mmol) in tetrahydrofuran (0.5 mL) and MeOH (0.5 mL). The mixture was heated with stirring to 70 degrees C. for 30 min. The crude reaction mixture was purified by reverse phase preparative HPLC (Prep HPLC Method 2) to provide 4-((1R,3aS,5aR,5bR,7aR,11aS,11bR,13aR,13bS)-1-(1-aminoethyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid Isomer 1 as a white powder TFA salt (0.040 g, 99% yield). LCMS: m/z=694.5 (M+H)⁺, 1.83 min (method 5). ¹H NMR (400 MHz, 1:1 mixture of chloroform-d and methanol-d₄, methanol-d₄ lock) δ 7.93 (d, J=8.3 Hz, 2H), 7.21 (d, J=8.3 Hz, 2H), 5.31 (d, J=4.9 Hz, 1H), 3.43 (q, J=6.6 Hz, 1H), 3.31-3.23 (m, 4H), 3.23-3.13 (m, 3H), 3.13-3.00 (m, 5H), 2.51 (t, J=9.7 Hz, 1H), 2.17 (dd, J=17.0, 6.2Hz, 1H), 2.11-2.02 (m, 2H), 2.02-1.91 (m, 3H), 1.89-1.77 (m, 2H), 1.74 (d, J=17.4 Hz, 1H), 1.68-1.53 (m, 7H), 1.52-1.44 (m, 4H), 1.43-1.35 (m, J=16.3, 7.5Hz, 1H), 1.30 (d, J=6.8 Hz, 4H), 1.22 (s, 3H), 1.12 (s, 3H), 1.05 (s, 3H), 0.97 (s, 3H), 0.96 (br. s., 3H).

### Example B20

### Preparation of 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(1-morpholinoethyl)-2,3,3a,4,5,5a,5b,6,7,7a,8,11,11a,11b,12,13,13a,13b-octadecahydro-1H-cyclopenta[a]chrysen-9-yl)benzoic acid.

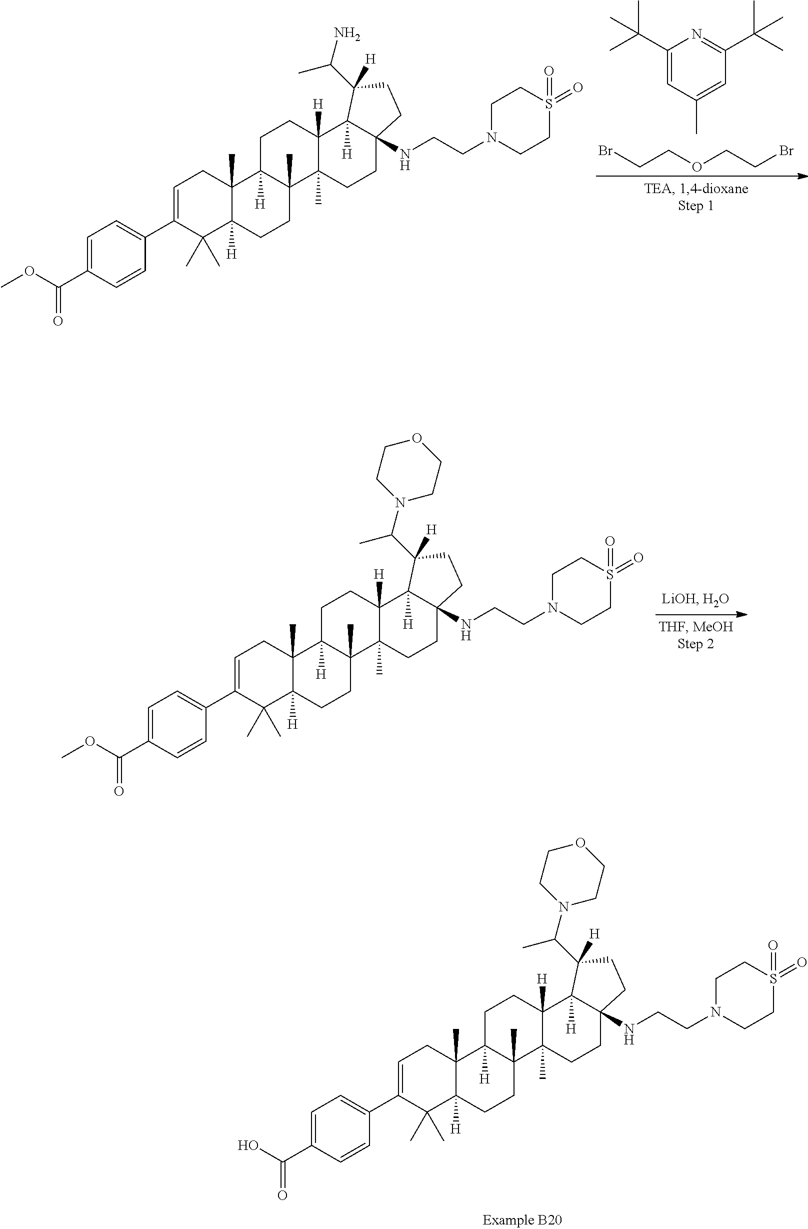

### Step 1. Preparation of methyl 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(1-morpholinoethyl)-2,3,3a,4,5,5a,5b,6,7,7a,8,11,11a,11b,12,13,13a,13b-octadecahydro-1H-cyclopenta[a]chrysen-9-yl)benzoate.

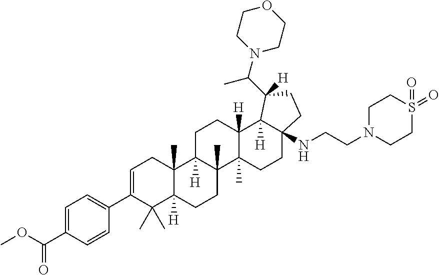

In a 20 mL scintillation vial were combined methyl 4-((1R,3aS,5aR,5bR,7aR,11aS,11bR,13aR,13bS)-1-(1-aminoethyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate TFA salt (0.020 g, 0.019 mmol) with 1-bromo-2-(2-bromoethoxy)ethane (0.00883 g, 0.038 mmol) and triethylamine (0.016 mL, 0.114 mmol) in 1,4-dioxane (0.5 mL). The mixture was heated to 85 degrees C. for 30 min which resulted in no reaction. The mixture was transferred to a 5 mL microwave vessel and was diluted with dry acetonitrile (2 mL). To the mixture was added additional 1-bromo-2-(2-bromoethoxy)ethane (another 10 equivalents; 0.0445 g, 0.190 mmol) as well as 2,6-di-tert-butyl-4-methylpyridine (0.023 g, 0.114 mmol). The resulting mixture was heated in the microwave to 120 degrees C. for 90 min. The contents of the vessel were concentrated under nitrogen stream, redissolved with a small quantity of a mixture of THF, acetonitrile and methanol, filtered and purified by reverse phase preparative HPLC (Prep HPLC Method 2). The desired product was thus obtained as a white solid and was carried directly into the next step. LCMS: m/z=778.6 (M+H)⁺, 2.13 min (method 5).

Step 2: In a 1 dram vial were combined methyl 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(1-morpholinoethyl)-2,3,3a,4,5,5a,5b,6,7,7a,8,11,11a,11b,12,13,13a,13b-octadecahydro-1H-cyclopenta[a]chrysen-9-yl)benzoate TFA salt (0.021 g, 0.019 mmol) with lithium hydroxide monohydrate, 1.0M aqueous solution (0.190 mL, 0.190 mmol) in tetrahydrofuran (0.5 mL) and MeOH (0.5 mL). The vial was sealed with a PTFE lined screwcap and the mixture was heated with stirring to 70° C. for 30 min. The crude mixture was purified by reverse phase preparative HPLC (Prep HPLC Method 14) to provide 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(1-morpholinoethyl)-2,3,3a,4,5,5a,5b,6,7,7a,8,11,11a,11b,12,13,13a,13b-octadecahydro-1H-cyclopenta[a]chrysen-9-yl)benzoic acid as a white powder TFA salt (0.0182 g, 86% yield over 2 steps). LCMS: m/z=764.6 (M+H)⁺, 1.85 min (method 5). ¹H NMR (400 MHz, 1:1 mixture of chloroform-d and methanol-d₄, methanol-d₄ lock) δ 7.93 (d, J=8.3 Hz, 2H), 7.21 (d, J=8.1 Hz, 2H), 5.31 (d, J=5.1 Hz, 1H), 3.88 (br. s., 4H), 3.30-2.96 (m, 16H), 2.41 (br. s., 2H), 2.17 (dd, J=17.0, 6.5Hz, 1H), 2.09 (d, J=15.7 Hz, 1H), 2.04-1.86 (m, 4H), 1.86-1.69 (m, 3H), 1.67-1.55 (m, 6H), 1.54-1.42 (m, 5H), 1.33-1.24 (m, 5H), 1.22 (s, 3H), 1.13 (s, 3H), 1.05 (s, 3H), 0.96 (br. s., 3H), 0.96 (br. s., 3H).

### Example B21

### Preparation of 4-((1R,3aS,5aR,5bR,7aR,11aS,11bR,13aR,13bS)-1-(1-acetamidoethyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid.

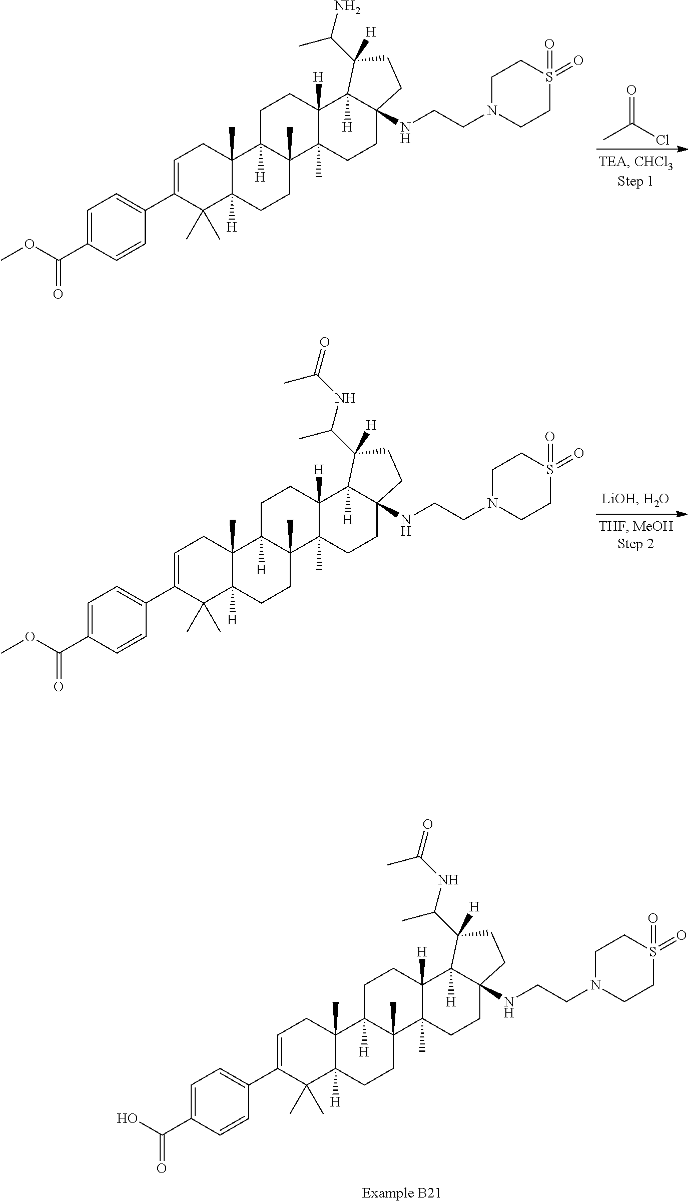

### Step 1. Preparation of methyl 4-((1R,3aS,5aR,5bR,7aR,11aS,11bR,13aR,13bS)-1-(1-acetamidoethyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate

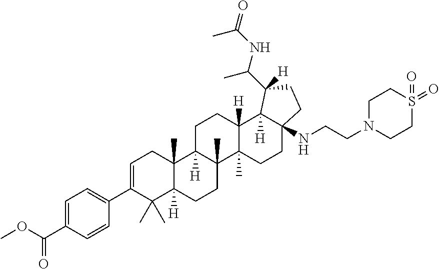

In a 1 dram vial with PTFE lined screwcap and stirbar were combined methyl 4-((1R,3aS,5aR,5bR,7aR,11aS,11bR,13aR,13bS)-1-(1-aminoethyl)-3a#2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate TFA salt (0.019 g, 0.018 mmol) with TEA (0.025 mL, 0.181 mmol) in dry chloroform (1 mL). Then acetyl chloride (1.930 μl, 0.027 mmol) was added all at once and the solution was stirred at rt for 30 min. The reaction mixture was concentrated under nitrogen stream and carried directly into the next step without purification. LCMS: m/z=750.5 (M+H)⁺, 2.17 min (method 5).

Step 2: In a 1 dram vial with PTFE lined screwcap were combined the crude mixture containing methyl 4-((1R,3aS,5aR,5bR,7aR,11aS,11bR,13aR,13bS)-1-(1-acetamidoethyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (0.014 g, 0.018 mmol) with lithium hydroxide monohydrate, 1.0M aqueous solution (0.180 mL, 0.180 mmol) in tetrahydrofuran (0.5 mL) and MeOH (0.5 mL). The mixture was heated with stirring to 70° C. for 30 min. The crude mixture was purified by reverse phase preparative HPLC in one injection (Prep HPLC Method 2). Thus was obtained 4-((1R,3aS,5aR,5bR,7aR,11aS,11bR,13aR,13bS)-1-(1-acetamidoethyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid as a white powder TFA salt (0.0108 g, 61.6% yield). LCMS: m/z=736.5 (M+H)⁺, 1.94 min (method 5). ¹H NMR (400 MHz, 1:1 mixture of chloroform-d and methanol-d₄, methanol-d₄ lock) δ 7.93 (d, J=8.3 Hz, 2H), 7.22 (d, J=8.3 Hz, 2H), 5.35-5.28 (m, 1H), 4.25 (quin, J=7.2Hz, 1H), 3.30-2.99 (m, 12H), 2.27-2.11 (m, 2H), 2.09-2.01 (m, 1H), 1.98 (s, 3H), 1.90-1.76 (m, 5H), 1.76-1.54 (m, 7H), 1.53-1.32 (m, 6H), 1.31-1.23 (m, 2H), 1.20 (s, 3H), 1.12 (d, J=6.8 Hz, 3H), 1.05 (s, 3H), 1.03 (s, 3H), 0.96 (s, 3H), 0.95 (s, 3H).

Example B22 and Example B23

### Preparation of 4-((1R,3aS,5aR,5bR,7aR,11aS,11bR,13aR,13bS)-3a-((2-(1,1-dioxidothiomorpholino)ethyl)amino)-1-(1-((methoxycarbonyl)amino)ethyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid and 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(1-(2,2,2-trifluoroacetamido)ethyl)-2,3,3a,4,5,5a,5b,6,7,7a,8,11,11a,11b,12,13,13a,13b-octadecahydro-1H-cyclopenta[a]chrysen-9-yl)benzoic acid.

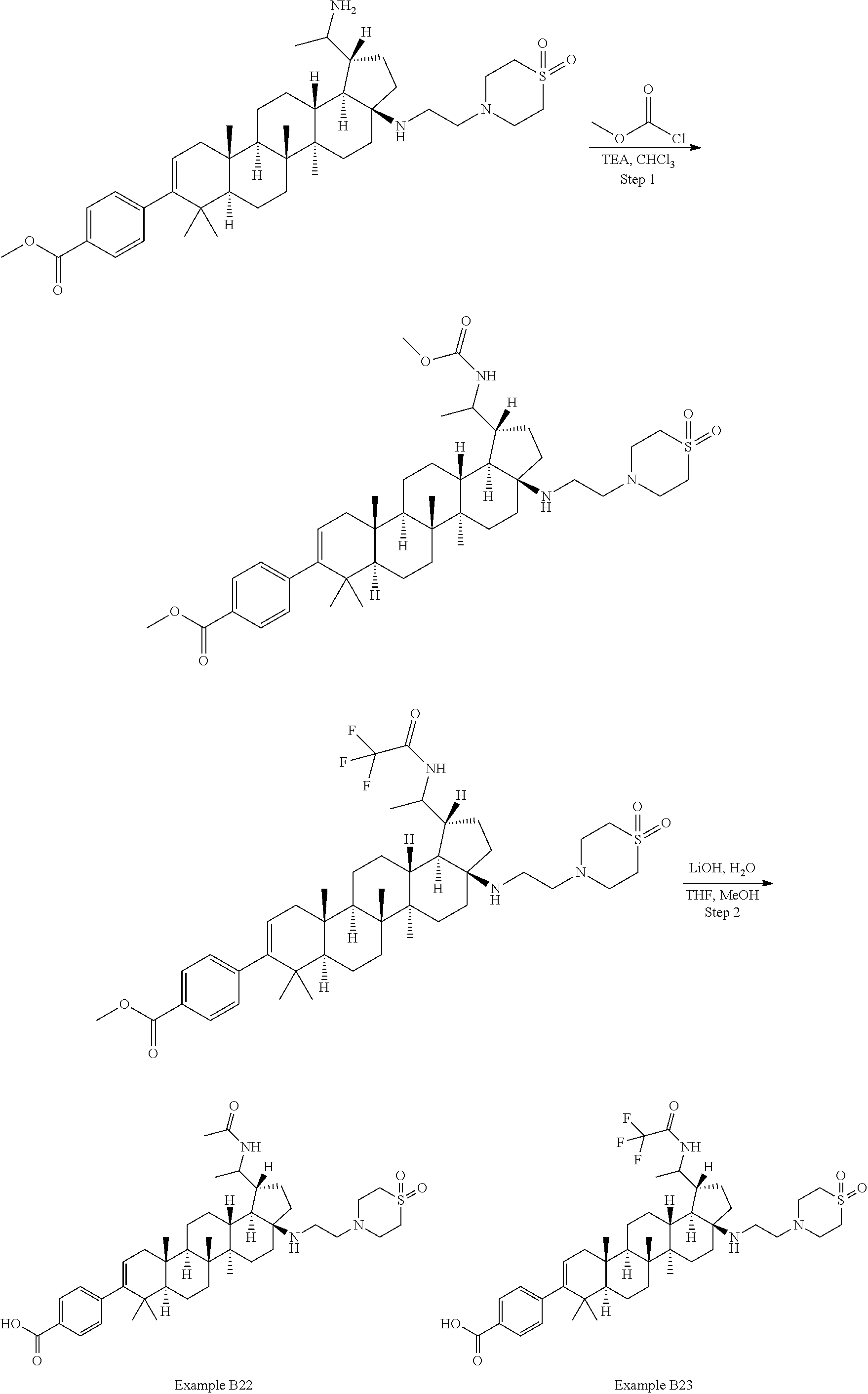

### Step 1. Preparation of methyl 4-((1R,3aS,5aR,5bR,7aR,11aS,11bR,13aR,13bS)-3a-((2-(1,1-dioxidothiomorpholino)ethyl)amino)-1-(1-((methoxycarbonyl)amino)ethyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate and methyl 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(1-(2,2,2-trifluoroacetamido)ethyl)-2,3,3a,4,5,5a,5b,6,7,7a,8,11,11a,11b,12,13,13a,13b-octadecahydro-1H-cyclopenta[a]chrysen-9-yl)benzoate.

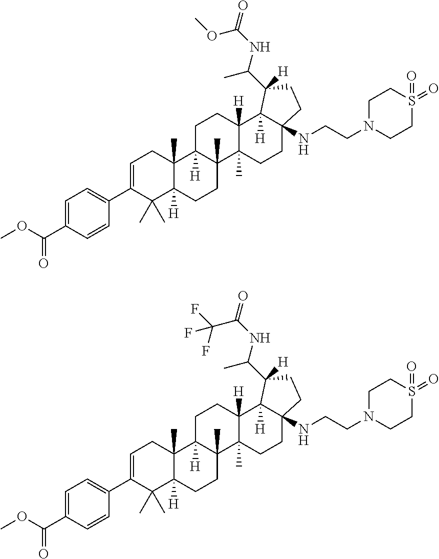

In a 20 mL scintillation vial with stirbar was placed methyl 4-((1R,3aS,5aR,5bR,7aR,11aS,11bR,13aR,13bS)-1-(1-aminoethyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate TFA salt (0.019 g, 0.018 mmol) with TEA (0.025 mL, 0.181 mmol) in dry chloroform (1 mL). To the mixture was added methyl chloroformate (2.102 μl, 0.027 mmol) and the solution was stirred at rt for 15 min. The crude reaction mixture was concentrated under nitrogen stream to a residue which was carried directly into the next step. LCMS: m/z=766.5 (M+H)⁺, 2.24 min and 804.5 (M+H)⁺, 2.32 min (method 5).

Step 2: In a 1 dram vial with PTFE screwcap were combined the crude mixture containing methyl 4-((1R,3aS,5aR,5bR,7aR,11aS,11bR,13aR,13bS)-3a-((2-(1,1-dioxidothiomorpholino)ethyl)amino)-1-(1-((methoxycarbonyl)amino)ethyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate and methyl 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(1-(2,2,2-trifluoroacetamido)ethyl)-2,3,3a,4,5,5a,5b,6,7,7a,8,11,11a,11b,12,13,13a,13b-octadecahydro-1H-cyclopenta[a]chrysen-9-yl)benzoate (0.018 mmol total) with lithium hydroxide monohydrate, 1.0M aqueous solution (0.180 mL, 0.180 mmol) in tetrahydrofuran (0.5 mL) and MeOH (0.5 mL). The mixture was heated with stirring to 70° C. for 25 min. The crude mixture was purified by reverse phase preparative HPLC (Prep HPLC Method 2). Thus were isolated the two title compounds: 4-((1R,3aS,5aR,5bR,7aR,11aS,11bR,13aR,13bS)-3a-((2-(1,1-dioxidothiomorpholino)ethyl)amino)-1-(1-((methoxycarbonyl)amino)ethyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid: Isolated as a white powder TFA salt (0.0039 g, 21.9% yield). LCMS: m/z=752.5 (M+H)⁺, 2.03 min (method 5). ¹H NMR (400 MHz, 1:1 mixture of chloroform-d and methanol-d₄, methanol-d₄ lock) δ 7.97-7.89 (m, 2H), 7.21 (d, J=8.3 Hz, 2H), 6.42 (d, J=9.3 Hz, 1H), 5.32 (dd, J=6.0, 1.3 Hz, 1H), 4.01-3.89 (m, 1H), 3.65 (s, 2H), 3.29-2.98 (m, 12H), 2.27 (d, J=8.1 Hz, 1H), 2.21-2.10 (m, 1H), 2.09-2.01 (m, 1H), 2.00-1.92 (m, 1H), 1.91-1.62 (m, 9H), 1.62-1.38 (m, 8H), 1.33-1.24 (m, 3H), 1.20 (s, 3H), 1.15 (d, J=6.8 Hz, 3H), 1.05 (s, 6H), 0.96 (br. s., 3H), 0.95 (br. s., 3H).

4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(1-(2,2,2-trifluoroacetamido)ethyl)-2,3,3a,4,5,5a,5b,6,7,7a,8,11,11a,11b,12,13,13a,13b-octadecahydro-1H-cyclopenta[a]chrysen-9-yl)benzoic acid: Isolated as a white powder TFA salt (0.0066 g, 35.7% yield). LCMS: m/z=790.5 (M+H)⁺, 2.12 min (method 5). ¹H NMR (400 MHz, METHANOL-d₄) δ 7.93 (d, J=8.1 Hz, 2H), 7.22 (d, J=8.3 Hz, 2H), 5.35-5.27 (m, 1H), 4.33 (t, J=8.3 Hz, 1H), 3.30 (br. s., 7H), 3.13-3.00 (m, 5H), 2.50-2.40 (m, 1H), 2.17 (dd, J=16.9, 6.4 Hz, 1H), 2.09-1.90 (m, 4H), 1.73 (d, J=2.2Hz, 3H), 1.71-1.55 (m, 6H), 1.55-1.42 (m, 5H), 1.37-1.26 (m, 3H), 1.24 (d, J=6.8 Hz, 3H), 1.19 (s, 3H), 1.05 (s, 3H), 1.02 (s, 3H), 0.96 (s, 3H), 0.96 (br. s., 3H).

### Example B24 and Example B25

### Preparation of 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(2-(sulfinooxy)acetyl)-2,3,3a,4,5,5a,5b,6,7,7a,8,11,11a,11b,12,13,13a,13b-octadecahydro-1H-cyclopenta[a]chrysen-9-yl)benzoic acid and 4-((1R,3aS,5aR,5bR,7aR,11aS,11bR,13aR,13bS)-3a-((2-(1,1-dioxidothiomorpholino)ethyl)amino)-1-(2-hydroxyacetyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid.

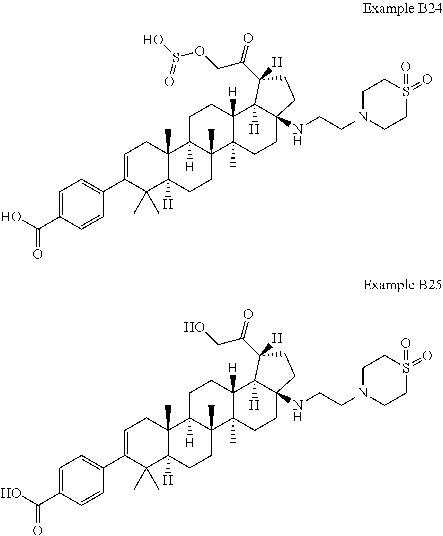

In a 1 dram vial with stirbar was placed sodium hydroxide, 3.0M aqueous (0.471 mL, 1.414 mmol). The vial was cooled to −10 degrees C. in an ice/acetone bath. To the stirred solution was added bromine (0.026 mL, 0.495 mmol) dropwise over 2 min. The resulting yellow/green solution was stirred for 10 min in the cold bath, then 1,4-dioxane (0.30 mL) was added dropwise very slowly and the resulting yellow solution was stirred another 5 min cold. The cold yellow hypobromite solution was added dropwise to a 0° C. cold frozen suspension of methyl 4-((1R,3aS,5aR,5bR,7aR,11aS,11bR,13aR,13bS)-1-acetyl-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (0.050 g, 0.071 mmol) in 1,4-dioxane (1.15 mL) and water (0.20 mL). The resulting yellow suspension was immediately allowed to warm to rt and was stirred at rt for 80 min. To the mixture was added 0.2 mL of a saturated aqueous sodium sulfite solution, and the mixture was heated to 80 degrees C. for 25 min. The mixture was concentrated to a solid residue under nitrogen stream. To the residue were added THF (2 mL), methanol (0.5 mL), water (0.3 mL) and acetonitrile (0.3 mL). The vial was shaken and the contents were filtered to remove solids. The crude mixture was purified by reverse phase preparative HPLC (Prep HPLC Method 2). Fractions containing the desired compounds were combined and repurified by reverse phase preparative HPLC (Prep HPLC Method 12). Thus were isolated the two title compounds: 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(2-(sulfinooxy)acetyl)-2,3,3a,4,5,5a,5b,6,7,7a,8,11,11a,11b,12,13,13a,13b-octadecahydro-1H-cyclopenta[a]chrysen-9-yl)benzoic acid: Isolated as a white powder (0.0091 g, 12.6% yield). LCMS: m/z=773.4 (M+H)⁺, 2.03 min (method 5). ¹H NMR (400 MHz, Acetic acid) δ 7.93 (d, J=8.1 Hz, 2H), 7.18 (d, J=8.1 Hz, 2H), 5.32 (d, J=5.4 Hz, 1H), 3.85 (td, J=10.8, 5.6 Hz, 1H), 3.47-3.08 (m, 13H), 2.67 (t, J=11.7 Hz, 1H), 2.57-2.40 (m, 1H), 2.18 (d, J=3.9 Hz, 3H), 1.99-1.93 (m, 2H), 1.84-1.71 (m, 3H), 1.70-1.38 (m, 12H), 1.33 (br. s., 3H), 1.28-1.19 (m, 4H), 1.16 (s, 3H), 1.09 (s, 3H), 0.98 (s, 3H), 0.96 (s, 3H). 4-((1R,3aS,5aR,5bR,7aR,11aS,11bR,13aR,13bS)-3a-((2-(1,1-dioxidothiomorpholino)ethyl)amino)-1-(2-hydroxyacetyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid: Isolated as a white powder (0.0042 g, 6.2% yield). LCMS: m/z=709.4 (M+H)⁺, 1.96 min (method 5). ¹H NMR (400 MHz, 1:1 mixture of chloroform-d and methanol-d₄, methanol-d₄ lock) δ 7.92 (d, J=8.1 Hz, 2H), 7.19 (d, J=8.3 Hz, 2H), 5.29 (d, J=4.6 Hz, 1H), 4.28 (d, J=4.4 Hz, 2H), 3.19-3.07 (m, 5H), 3.07-2.98 (m, 2H), 2.94 (td, J=10.9, 4.8 Hz, 2H), 2.78-2.58 (m, 3H), 2.45 (dt, J=11.6, 3.9 Hz, 1H), 2.20 (t, J=11.4 Hz, 1H), 2.15-2.04 (m, 2H), 2.01 (s, 1H), 1.98-1.82 (m, 2H), 1.75-1.63 (m, 2H), 1.62-1.20 (m, 12H), 1.20-1.06 (m, 5H), 1.04 (s, 3H), 1.00 (s, 3H), 0.95 (br. s., 3H), 0.94 (br. s., 3H).

### Example B26

### Preparation of 4-((1R,3aR,5aR,5bR,7aR,11aS,11bR,13aR,13bS)-3a-((2-(1,1-dioxidothiomorpholino)ethyl)amino)-1-((methoxycarbonyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid.

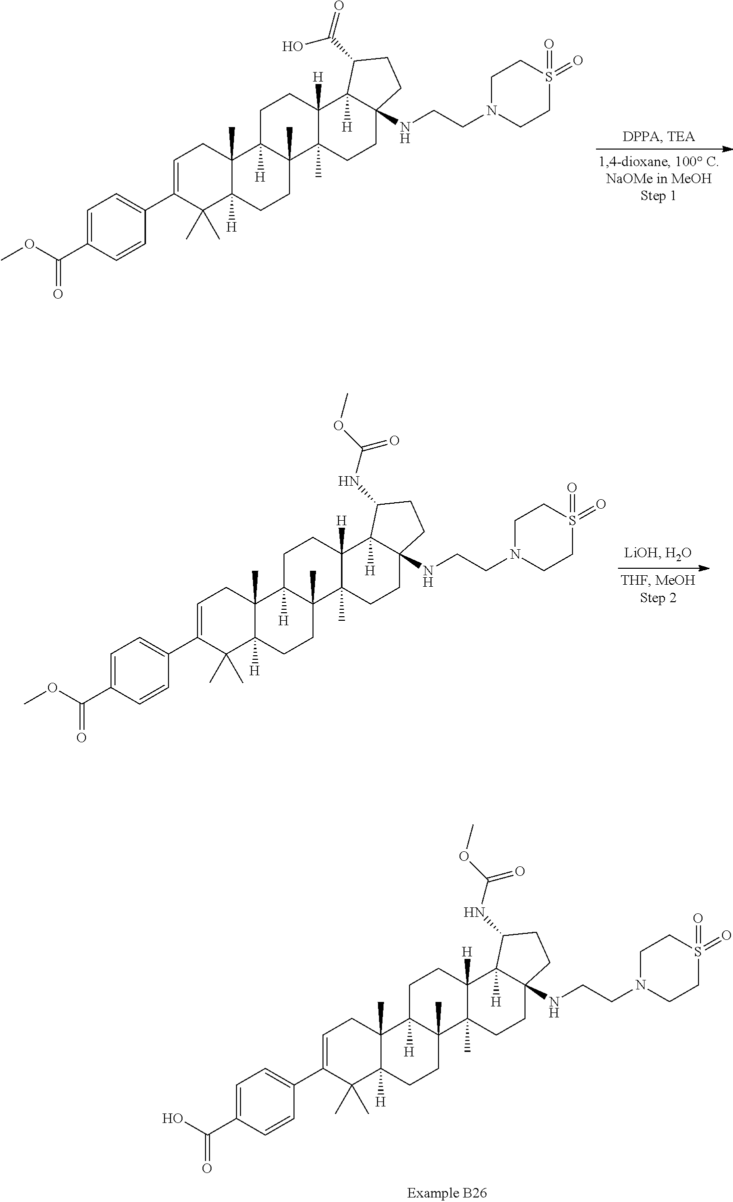

### Step 1. Preparation of methyl 4-((1R,3aR,5aR,5bR,7aR,11aS,11bR,13aR,13bS)-3a-((2-(1,1-dioxidothiomorpholino)ethyl)amino)-1-((methoxycarbonyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate.

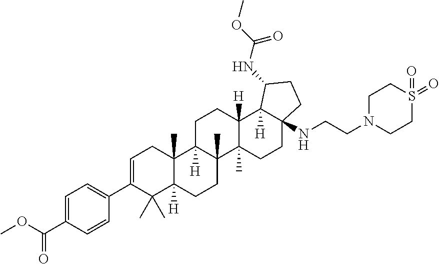

In a 1 dram vial were combined (1R,3aS,5aR,5bR,7aR,11aS,11bR,13aR,13bS)-3a-((2-(1,1-dioxidothiomorpholino)ethyl)amino)-9-(4-(methoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-1-carboxylic acid (0.025 g, 0.035 mmol) with triethylamine (8.85 μl, 0.063 mmol) and diphenylphosphoryl azide (0.011 mL, 0.053 mmol) in dry 1,4-dioxane (0.5 mL). The vial was sealed with a PTFE lined screwcap and the mixture was heated to 100 degrees C. for 75 min. To the mixture at rt was added sodium methoxide, 0.5M solution in methanol (0.705 mL, 0.353 mmol). After 1 h, the reaction mixture was concentrated via nitrogen stream and the crude residue was carried forward to hydrolysis of the ester in the next step. LCMS: m/z=738.7 (M+H)⁺, 2.25 min (method 3).

Step 2: In a 1 dram vial were combined methyl 4-((1R,3aR,5aR,5bR,7aR,11aS,11bR,13aR,13bS)-3a-((2-(1,1-dioxidothiomorpholino)ethyl)amino)-1-((methoxycarbonyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (0.026 g, 0.035 mmol) with lithium hydroxide monohydrate, 1.0M aqueous solution (0.352 mL, 0.352 mmol) and tetrahydrofuran (0.3 mL) and MeOH (0.3 mL). The vial was capped with PTFE screwcap and the mixture was heated to 70° C. with stirring for 20 min. The crude mixture was purified by reverse phase preparative HPLC (Prep HPLC Method 2) to give methyl 4-((1R,3aR,5aR,5bR,7aR,11aS,11bR,13aR,13bS)-3a-((2-(1,1-dioxidothiomorpholino)ethyl)amino)-1-((methoxycarbonyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate as a white powder TFA salt (0.0113 g, 32.7% yield). LCMS: m/z=724.4 (M+H)⁺, 1.96 min (method 3). ¹H NMR (400 MHz, 1:1 mixture of chloroform-d and methanol-d₄, methanol-d₄ lock) δ 7.93 (d, J=8.3 Hz, 2H), 7.21 (d, J=8.3 Hz, 2H), 6.87 (d, J=8.3 Hz, 1H), 5.30 (d, J=4.6 Hz, 1H), 4.11-3.96 (m, 1H), 3.64 (s, 3H), 3.27-2.87 (m, 12H), 2.38-2.23 (m, 1H), 2.22-2.04 (m, 3H), 2.03-1.82 (m, 3H), 1.76 (t, J=16.5Hz, 3H), 1.69-1.37 (m, 10H), 1.36-1.23 (m, 3H), 1.19 (s, 3H), 1.08 (s, 3H), 1.04 (s, 3H), 0.96 (br. s., 3H), 0.95 (br. s., 3H).

### Example B27

### Preparation of 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(morpholine-4-carbonyl)-2,3,3a,4,5,5a,5b,6,7,7a,8,11,11a,11b,12,13,13a,13b-octadecahydro-1H-cyclopenta[a]chrysen-9-yl)benzoic acid.

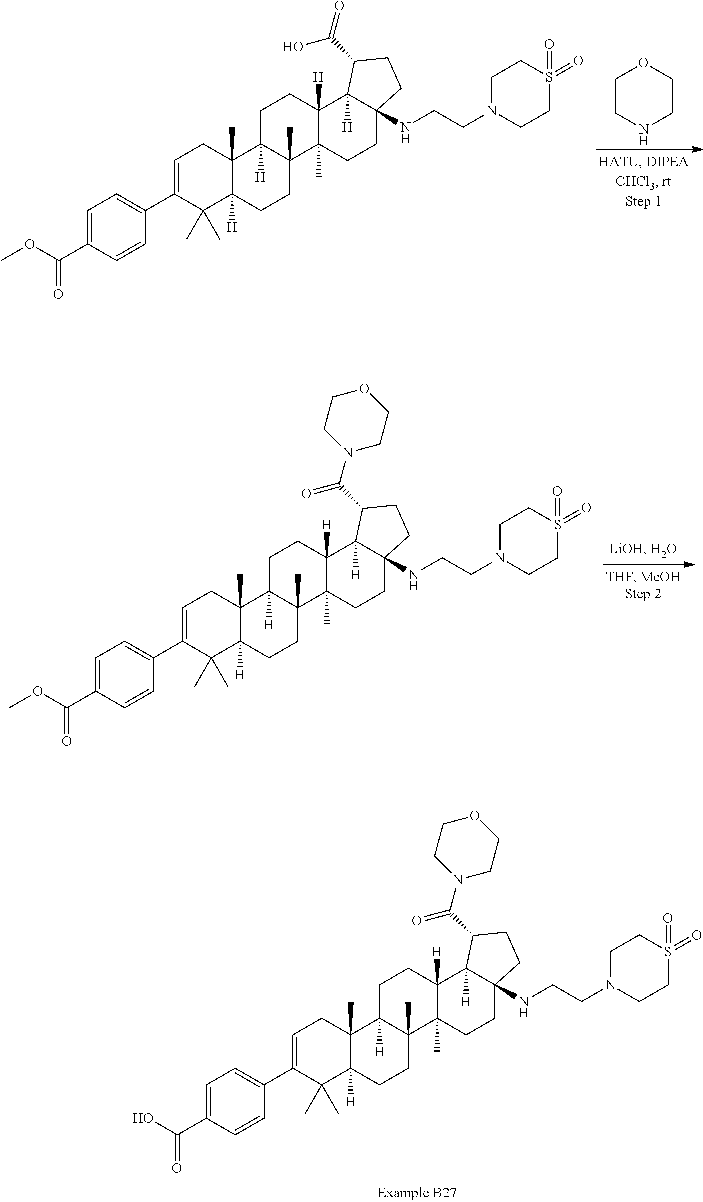

### Step 1. Preparation of methyl 4-((1R,3aS,5aR,5bR,7aR,11aS,11bR,13aR,13bS)-3a-((2-(1,1-dioxidothiomorpholino)ethypamino)-5a,5b,8,8,11a-pentamethyl-1-(morpholine-4-carbonyl)-2,3,3a,4,5,5a,5b,6,7,7a,8,11,11a,11b,12,13,13a,13b-octadecahydro-1H-cyclopenta[a]chrysen-9-yl)benzoate

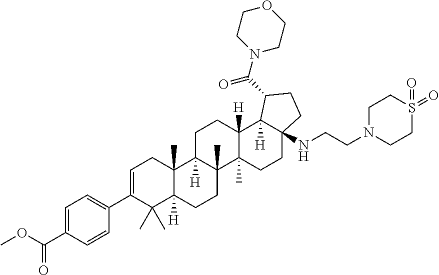

In a 1 dram vial were combined (1R,3aS,5aR,5bR,7aR,11aS,11bR,13aR,13bS)-3a-((2-(1,1-dioxidothiomorpholino)ethyl)amino)-9-(4-(methoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-1-carboxylic acid (0.025 g, 0.035 mmol) with morpholine (3.99 μl, 0.046 mmol), HATU (0.017 g, 0.046 mmol) and DIPEA (0.020 mL, 0.113 mmol) in chloroform (1 mL). The vial was sealed with a PTFE lined screwcap and the mixture was stirred at rt overnight. The mixture was concentrated under a nitrogen stream then redissolved in a minimum amount of a mixture of acetonitrile and methanol. The crude mixture was purified by reverse phase preparative HPLC (Prep HPLC method 5) to give the product as a white solid TFA salt (0.0276 g). LCMS: m/z=778.5 (M+H)⁺, 2.26 min (method 5). ¹H NMR (400 MHz, 1:1 mixture of chloroform-d and methanol-d₄, methanol-d₄ lock) δ 7.91 (d, J=8.1 Hz, 2H), 7.21 (d, J=8.3 Hz, 2H), 5.30 (d, J=4.6 Hz, 1H), 3.91 (s, 3H), 3.73-3.50 (m, 8H), 3.29-3.14 (m, 8H), 3.14-2.96 (m, 5H), 2.71 (t, J=11.7 Hz, 1H), 2.35-2.22 (m, 1H), 2.19-2.04 (m, 3H), 1.90-1.64 (m, 5H), 1.64-1.52 (m, 5H), 1.47 (d, J=12.0 Hz, 4H), 1.28 (d, J=10.3 Hz, 2H), 1.20 (s, 3H), 1.14 (s, 4H), 1.04 (s, 3H), 0.96 (s, 3H), 0.94 (s, 3H).

Step 2: In a 1 dram vial were combined methyl 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(morpholine-4-carbonyl)-2,3,3a,4,5,5a,5b,6,7,7a,8,11,11a,11b,12,13,13a,13b-octadecahydro-1H-cyclopenta[a]chrysen-9-yl)benzoate TFA salt (0.027 g, 0.027 mmol) with lithium hydroxide monohydrate, 1.0M aqueous solution (0.134 mL, 0.134 mmol) and tetrahydrofuran (0.3 mL) and MeOH (0.3 mL). The vial was sealed with a PTFE lined screwcap and the mixture was heated to 60° C. with stirring for 30 min. The crude mixture was purified by reverse phase preparative HPLC (Prep HPLC Method 2) to give 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(morpholine-4-carbonyl)-2,3,3a,4,5,5a,5b,6,7,7a,8,11,11a,11b,12,13,13a,13b-octadecahydro-1H-cyclopenta[a]chrysen-9-yl)benzoic acid as a white powder TFA salt (0.0323 g, 92% yield over 2 steps). LCMS: m/z=764.5 (M+H)⁺, 2.06 min (method 5). ¹H NMR (400 MHz, 1:1 mixture of chloroform-d and methanol-d₄, methanol-d₄ lock) δ 7.93 (d, J=8.3 Hz, 2H), 7.21 (d, J=8.3 Hz, 2H), 5.30 (d, J=4.6 Hz, 1H), 3.73-3.52 (m, 8H), 3.28-3.13 (m, 8H), 3.12-2.95 (m, 5H), 2.71 (t, J=11.6 Hz, 1H), 2.34-2.22 (m, 1H), 2.20-2.04 (m, 3H), 1.79 (d, J=4.2Hz, 2H), 1.65 (br. s., 3H), 1.64-1.35 (m, 9H), 1.33-1.23 (m, 2H), 1.21 (s, 3H), 1.14 (s, 4H), 1.04 (s, 3H), 0.96 (s, 3H), 0.95 (s, 3H).

### Example B28

### Preparation of 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(methylcarbamoyl)-2,3,3a,4,5,5a,5b,6,7,7a,8,11,11a,11b,12,13,13a,13b-octadecahydro-1H-cyclopenta[a]chrysen-9-yl)benzoic acid.

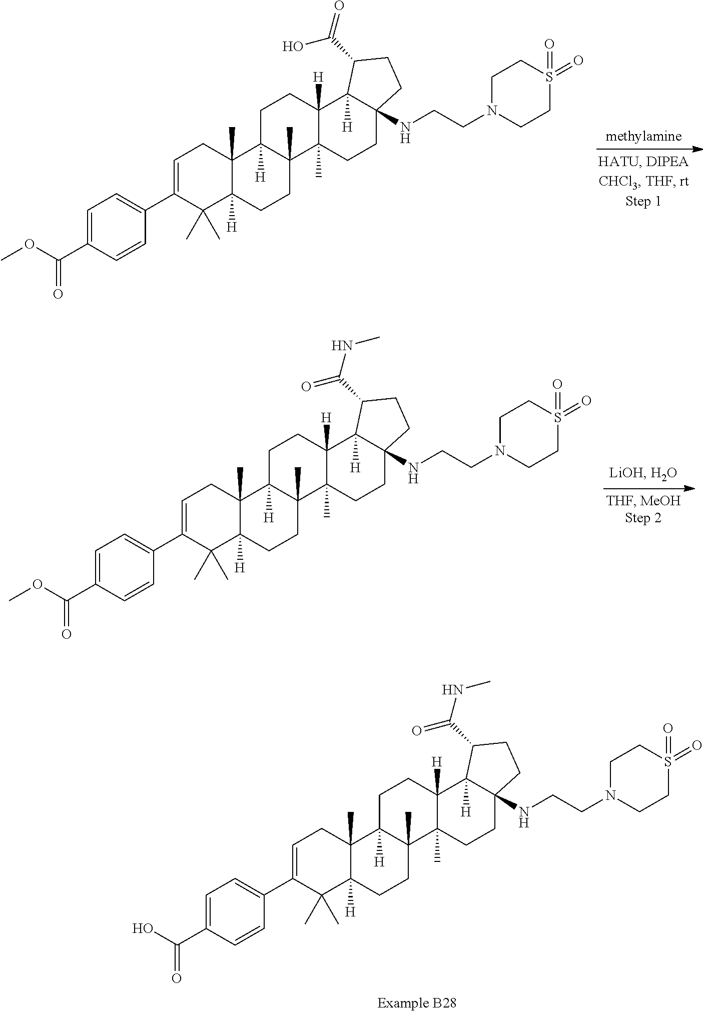

### Step 1. Preparation of methyl 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(methylcarbamoyl)-2,3,3a,4,5,5a,5b,6,7,7a,8,11,11a,11b,12,13,13a,13b-octadecahydro-1H-cyclopenta[a]chrysen-9-yl)benzoate.

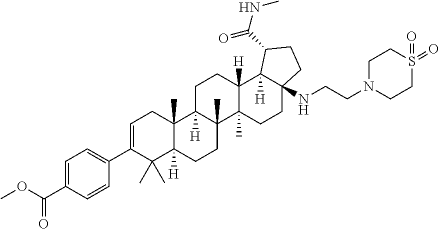

Methyl 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(methylcarbamoyl)-2,3,3a,4,5,5a,5b,6,7,7a,8,11,11a,11b,12,13,13a,13b-octadecahydro-1H-cyclopenta[a]chrysen-9-yl)benzoate was prepared by a similar procedure as described for the preparation of methyl 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(morpholine-4-carbonyl)-2,3,3a,4,5,5a,5b,6,7,7a,8,11,11a,11b,12,13,13a,13b-octadecahydro-1H-cyclopenta[a]chrysen-9-yl)benzoate, except methylamine, 2.0M in THF (0.176 mL, 0.353 mmol) was used instead of morpholine. Also, after preparative HPLC purification the material had to be repurified using different conditions (prep HPLC method 12) to provide the desired product as a white powder (0.0142 g, 55.8% yield). LCMS: m/z=722.6 (M+H)⁺, 2.02 min (method 3). ¹H NMR (400 MHz, 1:1 mixture of chloroform-d and methanol-d₄, methanol-d₄ lock) δ 7.91 (d, J=8.1 Hz, 2H), 7.44-7.36 (m, 1H), 7.21 (d, J=8.3 Hz, 2H), 5.29 (d, J=4.6 Hz, 1H), 3.90 (s, 3H), 3.19-2.95 (m, 8H), 2.75-2.68 (m, 5H), 2.67-2.58 (m, 1H), 2.57-2.40 (m, 2H), 2.16-1.88 (m, 4H), 1.83 (dd, J=12.8, 7.9 Hz, 1H), 1.78-1.66 (m, 2H), 1.65-1.42 (m, 8H), 1.42-1.29 (m, 4H), 1.29-1.21 (m, 2H), 1.21-1.14 (m, 1H), 1.12 (s, 3H), 1.03 (s, 3H), 1.00 (s, 3H), 0.94 (s, 3H), 0.93 (s, 3H).

Step 2: In a 1 dram vial were combined methyl 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(methylcarbamoyl)-2,3,3a,4,5,5a,5b,6,7,7a,8,11,11a,11b,12,13,13a,13b-octadecahydro-1H-cyclopenta[a]chrysen-9-yl)benzoate (0.0142 g, 0.020 mmol) with lithium hydroxide monohydrate, 1.0M aqueous solution (0.197 mL, 0.197 mmol) and tetrahydrofuran (0.3 mL) and MeOH (0.3 mL). The vial was sealed with a PTFE lined screwcap and the mixture was heated to 60° C. with stirring for 30 min. The crude mixture was purified by reverse phase preparative HPLC (Prep HPLC Method 2) to give 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(methylcarbamoyl)-2,3,3a,4,5,5a,5b,6,7,7a,8,11,11a,11b,12,13,13a,13b-octadecahydro-1H-cyclopenta[a]chrysen-9-yl)benzoic acid as a white powder TFA salt (0.0171 mg, 92% yield). LCMS: m/z=708.4 (M+H)⁺, 1.98 min (method 5). ¹H NMR (400 MHz, 1:1 mixture of chloroform-d and methanol-d₄, methanol-d₄ lock) δ 7.92 (d, J=8.3 Hz, 2H), 7.20 (d, J=8.3 Hz, 2H), 5.30 (d, J=4.6 Hz, 1H), 3.37 (s, 3H), 3.26-3.04 (m, 10H), 3.00 (d, J=4.2Hz, 2H), 2.85 (br. s., 1H), 2.75 (s, 3H), 2.51 (t, J=11.6 Hz, 1H), 2.21-2.07 (m, 3H), 2.06-1.97 (m, 1H), 1.90-1.67 (m, 5H), 1.65-1.34 (m, 10H), 1.21 (br. s., 2H), 1.18 (s, 3H), 1.10 (s, 3H), 1.03 (s, 3H), 0.96 (s, 3H), 0.94 (s, 3H).

### Example B29

### Preparation of 4-((1R,3aR,5aR,5bR,7aR,11aS,11bR,13aR,13bS)-3a-((2-(1,1-dioxidothiomorpholino)ethyl)amino)-5a,5b,8,8,11a-pentamethyl-1-(3-methylureido)-2,3,3a,4,5,5a,5b,6,7,7a,8,11,11a,11b,12,13,13a,13b-octadecahydro-1H-cyclopenta[a]chrysen-9-yl)benzoic acid.

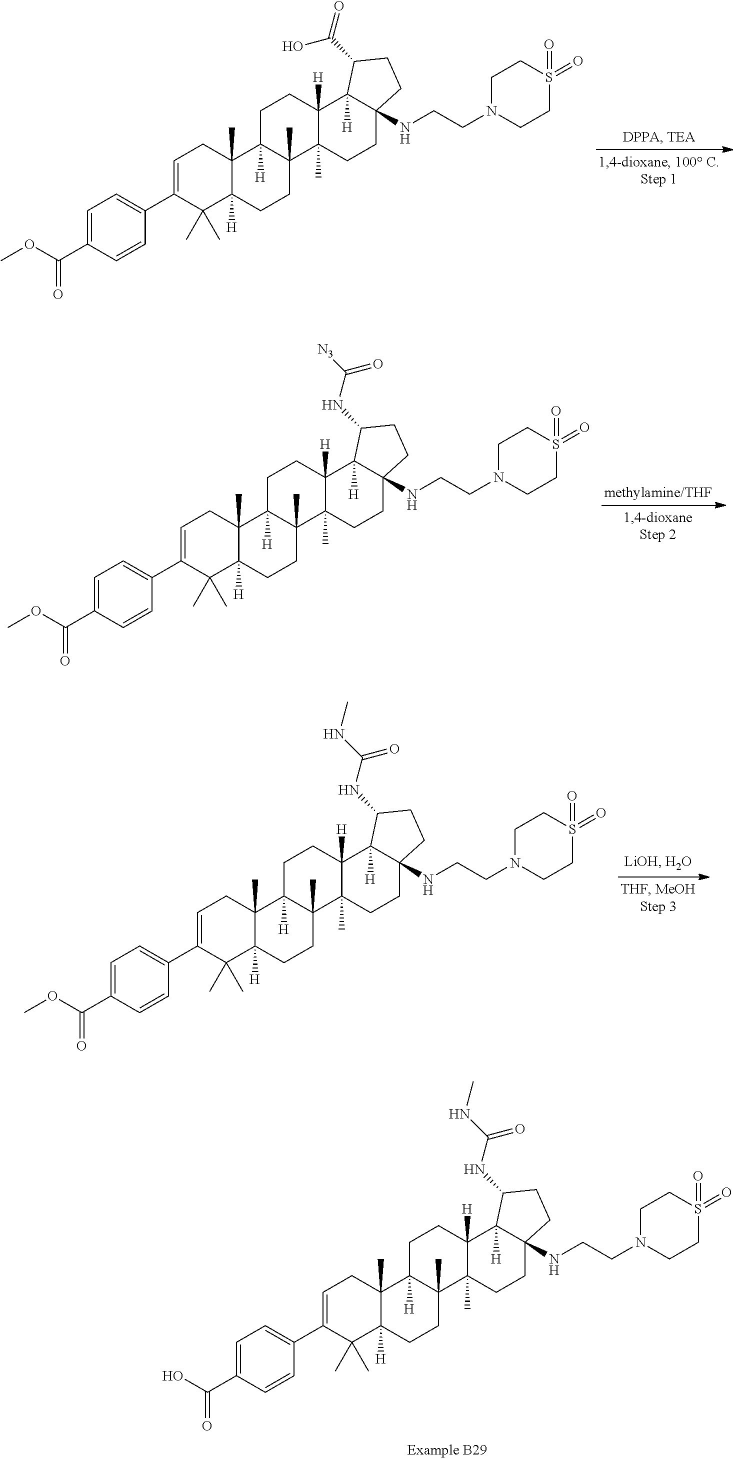

### Step 1. Preparation of methyl 4-((1R,3aR,5aR,5bR,7aR,11aS,11bR,13aR,13bS)-1-((azidocarbonyl)amino)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate.

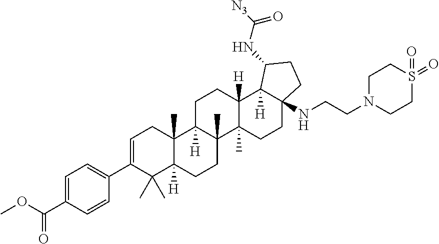

In a 1 dram vial were combined (1R,3aS,5aR,5bR,7aR,11aS,11bR,13aR,13bS)-3a-((2-(1,1-dioxidothiomorpholino)ethyl)amino)-9-(4-(methoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-1-carboxylic acid (0.085 g, 0.120 mmol) with triethylamine (0.030 mL, 0.216 mmol) and diphenylphosphoryl azide (0.028 mL, 0.132 mmol) in dry 1,4-dioxane (2 mL). The vial was sealed with a PTFE lined screwcap and the mixture was stirred at rt for 100 min, then heated to 100° C. and stirred for 2 h. To the mixture was added more diphenylphosphoryl azide (0.028 mL, 0.132 mmol) and the mixture was reheated to 100° C. and stirred for 1 h. The crude mixture was taken directly into the next step without purification. LCMS: m/z=749.6 (M+H)⁺, 2.30 min (method 3).

### Step 2. Preparation of methyl 4-((1R,3aR,5aR,5bR,7aR,11aS,11bR,13aR,13bS)-3a-((2-(1,1-dioxidothiomorpholino)ethyl)amino)-5a,5b,8,8,11a-pentamethyl-1-(3-methylureido)-2,3,3a,4,5,5a,5b,6,7,7a,8,11,11a,11b,12,13,13a,13b-octadecahydro-1H-cyclopenta[a]chrysen-9-yl)benzoate.

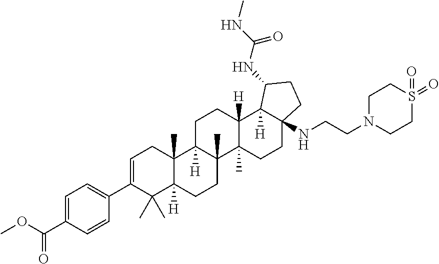

In a 1 dram vial were combined methyl 4-((1R,3aR,5aR,5bR,7aR,11aS,11bR,13aR,13bS)-1-((azidocarbonyl)amino)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (0.030 g, 0.040 mmol) with methanamine, 1.0M in THF (0.400 mL, 0.400 mmol). The mix was stirred at rt for 1 h. The crude mixture was purified by reverse phase preparative HPLC (Prep HPLC Method 2) to provide the desired product as a white powder TFA salt (0.0193 g, 50% yield over 2 steps). LCMS: m/z=737.4 (M+H)⁺, 2.14 min (method 5). ¹H NMR (400 MHz, 1:1 mixture of chloroform-d and methanol-d₄, methanol-d₄ lock) δ 7.91 (d, J=8.1 Hz, 2H), 7.21 (d, J=8.1 Hz, 2H), 5.30 (d, J=4.9 Hz, 1H), 4.12-4.01 (m, 1H), 3.91 (s, 3H), 3.31-3.01 (m, 11H), 2.97 (d, J=11.2Hz, 1H), 2.71 (s, 3H), 2.36-2.21 (m, 1H), 2.15 (dd, J=17.1, 6.4 Hz, 1H), 2.11-2.02 (m, 2H), 2.01-1.92 (m, 1H), 1.91-1.79 (m, 2H), 1.79-1.37 (m, 13H), 1.33-1.22 (m, 2H), 1.18 (s, 3H), 1.09 (s, 3H), 1.04 (s, 3H), 0.96 (s, 3H), 0.94 (s, 3H).

Step 3: In a 1 dram vial were combined methyl 4-((1R,3aR,5aR,5bR,7aR,11aS,11bR,13aR,13bS)-3a-((2-(1,1-dioxidothiomorpholino)ethyl)amino)-5a,5b,8,8,11a-pentamethyl-1-(3-methylureido)-2,3,3a,4,5,5a,5b,6,7,7a,8,11,11a,11b,12,13,13a,13b-octadecahydro-1H-cyclopenta[a]chrysen-9-yl)benzoate TFA salt (0.019 g, 0.020 mmol) with lithium hydroxide monohydrate, 1.0M aqueous solution (0.197 mL, 0.197 mmol) and tetrahydrofuran (0.3 mL) and MeOH (0.3 mL). The vial was sealed with a PTFE lined screwcap and the mixture was heated to 60° C. with stirring for 30 min. The crude mixture was purified by reverse phase preparative HPLC (Prep HPLC Method 2) to provide 4-((1R,3aR,5aR,5bR,7aR,11aS,11bR,13aR,13bS)-3a-((2-(1,1-dioxidothiomorpholino)ethyl)amino)-5a,5b,8,8,11a-pentamethyl-1-(3-methylureido)-2,3,3a,4,5,5a,5b,6,7,7a,8,11,11a,11b,12,13,13a,13b-octadecahydro-1H-cyclopenta[a]chrysen-9-yl)benzoic acid as a white powder TFA salt (0.0203 g, 106% yield). LCMS: m/z=723.4 (M+H)⁺, 1.93 min (method 5). ¹H NMR (400 MHz, 1:1 mixture of chloroform-d and methanol-d₄, methanol-d₄ lock) δ 7.93 (d, J=8.3 Hz, 2H), 7.20 (d, J=8.3 Hz, 2H), 5.30 (d, J=4.6 Hz, 1H), 4.16-3.99 (m, 1H), 3.28-3.00 (m, 11H), 3.00-2.86 (m, 1H), 2.72 (s, 3H), 2.38-2.21 (m, 1H), 2.16 (dd, J=17.2, 6.5Hz, 1H), 2.12-2.01 (m, 2H), 2.01-1.93 (m, 1H), 1.90-1.55 (m, 10H), 1.35 (d, J=11.7 Hz, 6H), 1.29 (d, J=10.8 Hz, 2H), 1.18 (s, 3H), 1.09 (s, 3H), 1.04 (s, 3H), 0.96 (br. s., 3H), 0.95 (br. s., 3H).

### Example B30

### Preparation of 4-((1R,3aR,5aR,5bR,7aR,11aS,11bR,13aR,13bS)-3a-((2-(1,1-dioxidothiomorpholino)ethyl)amino)-5a,5b,8,8,11a-pentamethyl-1-(pyrrolidine-1-carboxamido)-2,3,3a,4,5,5a,5b,6,7,7a,8,11,11a,11b,12,13,13a,13b-octadecahydro-1H-cyclopenta[a]chrysen-9-yl)benzoic acid.

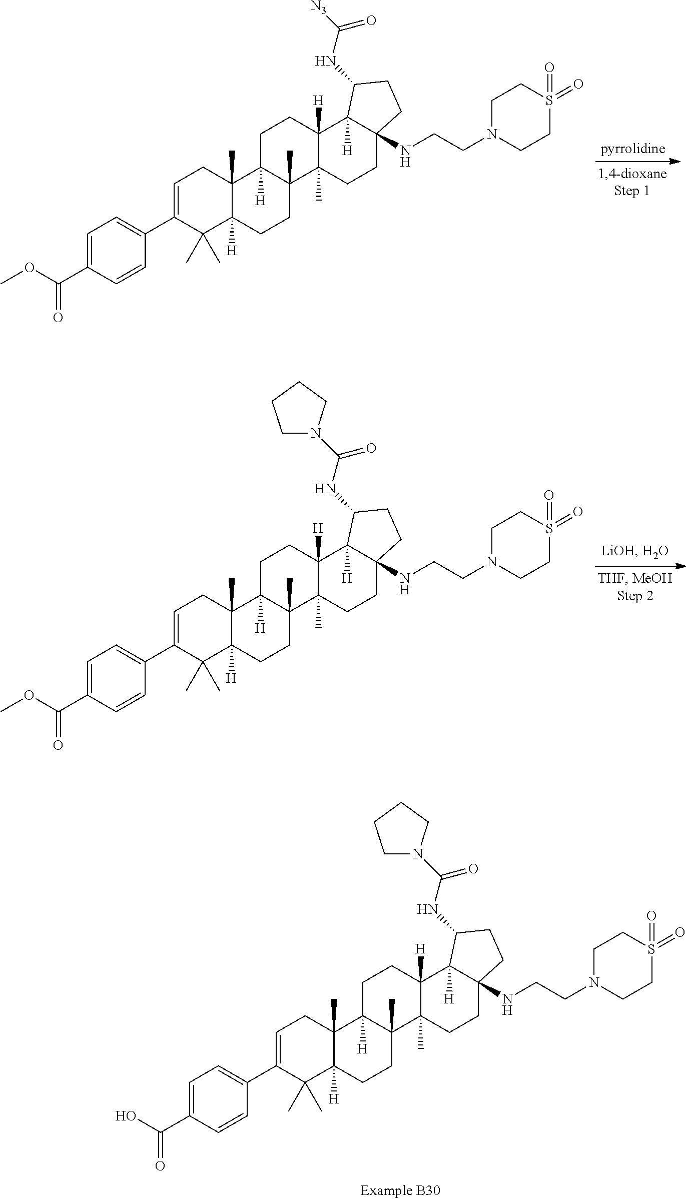

### Step 1. Preparation of methyl 4-((1R,3aR,5aR,5bR,7aR,11aS,11bR,13aR,13bS)-3a-((2-(1,1-dioxidothiomorpholino)ethyl)amino)-5a,5b,8,8,11a-pentamethyl-1-(pyrrolidine-1-carboxamido)-2,3,3a,4,5,5a,5b,6,7,7a,8,11,11a,11b,12,13,13a,13b-octadecahydro-1H-cyclopenta[a]chrysen-9-yl)benzoate.

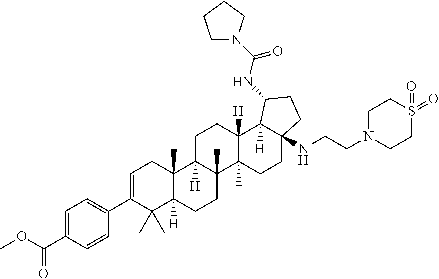

In a 1 dram vial were combined methyl 4-((1R,3aR,5aR,5bR,7aR,11aS,11bR,13aR,13bS)-1-((azidocarbonyl)amino)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (0.030 g, 0.040 mmol) with pyrrolidine (0.033 mL, 0.400 mmol). The vial was sealed with a PTFE lined screwcap and the mixture was stirred at rt for 1 h. The crude mixture was purified by reverse phase preparative HPLC (Prep HPLC Method 2). The desired product was thus obtained as a white powder TFA salt (0.0178 g, 44.3% yield). LCMS: m/z=777.5 (M+H)⁺, 2.16 min (method 5). ¹H NMR (400 MHz, 1:1 mixture of chloroform-d and methanol-d₄, methanol-d₄ lock) δ 7.91 (d, J=8.3 Hz, 2H), 7.21 (d, J=8.3 Hz, 2H), 5.30 (d, J=4.9 Hz, 1H), 4.16 (br. s., 1H), 3.91 (s, 3H), 3.31-3.00 (m, 12H), 2.94 (br. s., 1H), 2.38-2.21 (m, 2H), 2.16 (dd, J=17.0, 6.2 Hz, 1H), 2.09 (d, J=14.7 Hz, 1H), 2.01-1.66 (m, 12H), 1.65-1.34 (m, 10H), 1.33-1.22 (m, 2H), 1.18 (s, 3H), 1.10 (s, 3H), 1.04 (s, 3H), 0.96 (br. s., 3H), 0.95 (br. s., 3H).

Step 2: In a 1 dram vial were combined methyl 4-((1R,3aR,5aR,5bR,7aR,11aS,11bR,13aR,13bS)-3a-((2-(1,1-dioxidothiomorpholino)ethyl)amino)-5a,5b,8,8,11a-pentamethyl-1-(pyrrolidine-1-carboxamido)-2,3,3a,4,5,5a,5b,6,7,7a,8,11,11a,11b,12,13,13a,13b-octadecahydro-1H-cyclopenta[a]chrysen-9-yl)benzoate TFA salt (0.019 g, 0.019 mmol) with lithium hydroxide monohydrate, 1.0M aqueous solution (0.189 mL, 0.189 mmol) and tetrahydrofuran (0.3 mL) and MeOH (0.3 mL). The vial was sealed with a PTFE lined screwcap and the mixture was heated to 60° C. with stirring for 30 min. The crude mixture was purified by reverse phase preparative HPLC (Prep HPLC Method 2) to provide 4-((1R,3aR,5aR,5bR,7aR,11aS,11bR,13aR,13bS)-3a-((2-(1,1-dioxidothiomorpholino)ethyl)amino)-5a,5b,8,8,11a-pentamethyl-1-(pyrrolidine-1-carboxamido)-2,3,3a,4,5,5a,5b,6,7,7a,8,11,11a,11b,12,13,13a,13b-octadecahydro-1H-cyclopenta[a]chrysen-9-yl)benzoic acid as a white powder TFA salt (0.0163 g, 85% yield). LCMS: m/z=763.4 (M+H)⁺, 2.04 min (method 5). ¹H NMR (400 MHz, 1:1 mixture of chloroform-d and methanol-d₄, methanol-d₄ lock) δ 7.93 (d, J=8.3 Hz, 2H), 7.20 (d, J=8.3 Hz, 2H), 5.30 (d, J=4.9 Hz, 1H), 4.21-4.08 (m, 1H), 3.27-3.00 (m, 12H), 2.93 (d, J=9.3 Hz, 1H), 2.28 (t, J=11.4 Hz, 2H), 2.16 (dd, J=17.1, 6.4 Hz, 1H), 2.09 (d, J=15.4 Hz, 1H), 1.99-1.90 (m, 5H), 1.90-1.80 (m, 2H), 1.79-1.66 (m, 4H), 1.66-1.54 (m, 4H), 1.54-1.34 (m, 6H), 1.33-1.22 (m, 3H), 1.18 (s, 3H), 1.10 (s, 3H), 1.04 (s, 3H), 0.96 (br. s., 3H), 0.95 (br. s., 3H).

### Example B31

### Preparation of 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(1,3,4-oxadiazol-2-yl)-2,3,3a,4,5,5a,5b,6,7,7a,8,11,11a,11b,12,13,13a,13b-octadecahydro-1H-cyclopenta[a]chrysen-9-yl)benzoic acid.

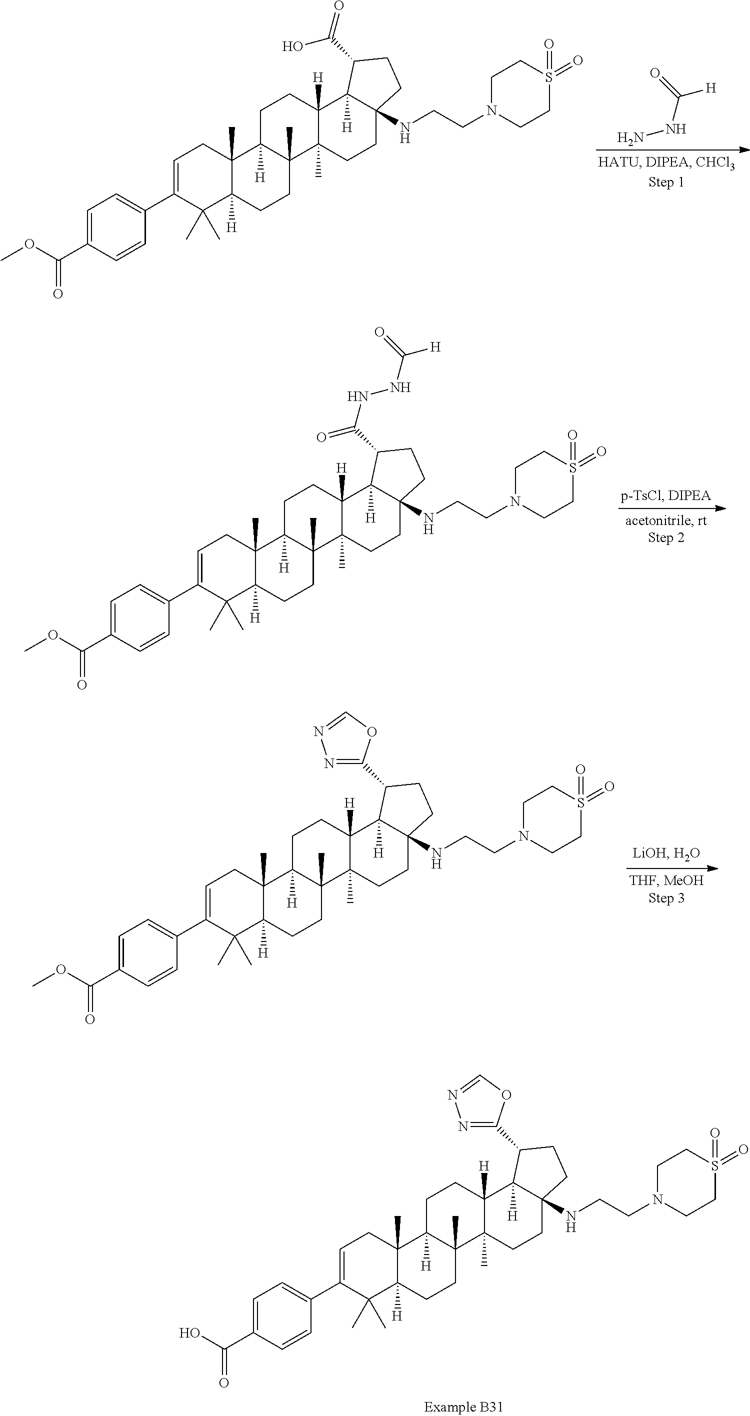

### Step 1. Preparation of methyl 4-((1R,3aS,5aR,5bR,7aR,11aS,11bR,13aR,13bS)-3a-((2-(1,1-dioxidothiomorpholino)ethyl)amino)-1-(2-formylhydrazinecarbonyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate.

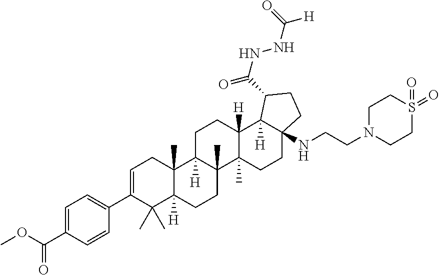

In a 1 dram vial were combined (1R,3aS,5aR,5bR,7aR,11aS,11bR,13aR,13bS)-3a-((2-(1,1-dioxidothiomorpholino)ethyl)amino)-9-(4-(methoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-1-carboxylic acid (0.050 g, 0.071 mmol) with formic acid hydrazide (8.47 mg, 0.141 mmol), HATU (0.035 g, 0.092 mmol) and DIPEA (0.039 mL, 0.226 mmol) in chloroform (1 mL). The mixture was stirred at rt for 26 h. The mixture was concentrated via nitrogen stream, then redissolved in a minimum quantity of a mixture of acetonitrile/MeOH, filtered and purified by reverse phase preparative HPLC (Prep HPLC Method 2). The desired product was thus isolated as a white solid TFA salt (0.0396 g, 57.4% yield). LCMS: m/z=751.4 (M+H)⁺, 2.11 min (method 5). ¹H NMR (400 MHz, 1:1 mixture of chloroform-d and methanol-d₄, methanol-d₄ lock) δ 8.04 (s, 1H), 7.91 (d, J=8.3 Hz, 2H), 7.21 (d, J=8.3 Hz, 2H), 5.29 (d, J=4.6 Hz, 1H), 3.91 (s, 3H), 3.27-3.04 (m, 10H), 3.03-2.88 (m, 3H), 2.57 (t, J=11.9 Hz, 1H), 2.32-2.18 (m, 1H), 2.18-2.02 (m, 3H), 1.95-1.65 (m, 5H), 1.64-1.32 (m, 10H), 1.31-1.21 (m, 2H), 1.18 (s, 3H), 1.10 (s, 3H), 1.03 (s, 3H), 0.95 (s, 3H), 0.94 (s, 3H).

### Step 2. Preparation of methyl 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(1,3,4-oxadiazol-2-yl)-2,3,3a,4,5,5a,5b,6,7,7a,8,11,11a,11b,12,13,13a,13b-octadecahydro-1H-cyclopenta[a]chrysen-9-yl)benzoate.

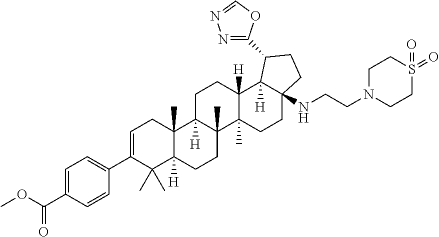

In a 1 dram vial were combined methyl 4-((1R,3aS,5aR,5bR,7aR,11aS,11bR,13aR,13bS)-3a-((2-(1,1-dioxidothiomorpholino)ethyl)amino)-1-(2-formylhydrazinecarbonyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate TFA salt (0.020 g, 0.020 mmol) and DIPEA (0.036 mL, 0.204 mmol) with acetonitrile (0.5 mL). To the mixture was added p-toluenesulfonyl chloride (0.031 g, 0.163 mmol). The mixture was stirred at rt for 1 h. The crude mixture was purified by reverse phase preparative HPLC (Prep HPLC Method 12). Thus was obtained the desired product (0.0084 g, 56.1% yield). LCMS: m/z=733.7 (M+H)⁺, 2.32 min (method 3).

Step 3: In a 1 dram vial were combined methyl 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(1,3,4-oxadiazol-2-yl)-2,3,3a,4,5,5a,5b,6,7,7a,8,11,11a,11b,12,13,13a,13b-octadecahydro-1H-cyclopenta[a]chrysen-9-yl)benzoate (0.0084 g, 0.011 mmol) with lithium hydroxide monohydrate, 1.0M aqueous solution (0.115 mL, 0.115 mmol) and tetrahydrofuran (0.3 mL) and MeOH (0.3 mL). The vial was sealed with a PTFE lined screwcap and the mixture was heated to 70° C. with stirring for 30 min. The crude mixture was purified by reverse phase preparative HPLC (Prep HPLC Method 3) to afford 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(1,3,4-oxadiazol-2-yl)-2,3,3a,4,5,5a,5b,6,7,7a,8,11,11a,11b,12,13,13a,13b-octadecahydro-1H-cyclopenta[a]chrysen-9-yl)benzoic acid as a white powder TFA salt (0.0106 g, 97% yield). LCMS: m/z=719.4 (M+H)⁺, 1.92 min (method 5). ¹H NMR (400 MHz, 1:1 mixture of chloroform-d and methanol-d₄, methanol-d₄ lock) δ 8.70 (s, 1H), 7.92 (d, J=8.3 Hz, 2H), 7.19 (d, J=8.3 Hz, 2H), 5.32-5.21 (m, 1H), 3.75 (td, J=11.1, 3.7 Hz, 1H), 3.27-2.92 (m, 11H), 2.65 (t, J=12.0 Hz, 1H), 2.57-2.42 (m, 1H), 2.26-1.76 (m, 7H), 1.72-1.33 (m, 10H), 1.26 (br. s., 3H), 1.20 (s, 3H), 1.16-1.05 (m, 4H), 1.02 (s, 3H), 0.95 (s, 3H), 0.94 (s, 3H), 0.91-0.84 (m, 1H).

### Example B32

### Preparation of 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(1-(2-oxopyrrolidin-1-yl)ethyl)-2,3,3a,4,5,5a,5b,6,7,7a,8,11,11a,11b,12,13,13a,13b-octadecahydro-1H-cyclopenta[a]chrysen-9-yl)benzoic acid.

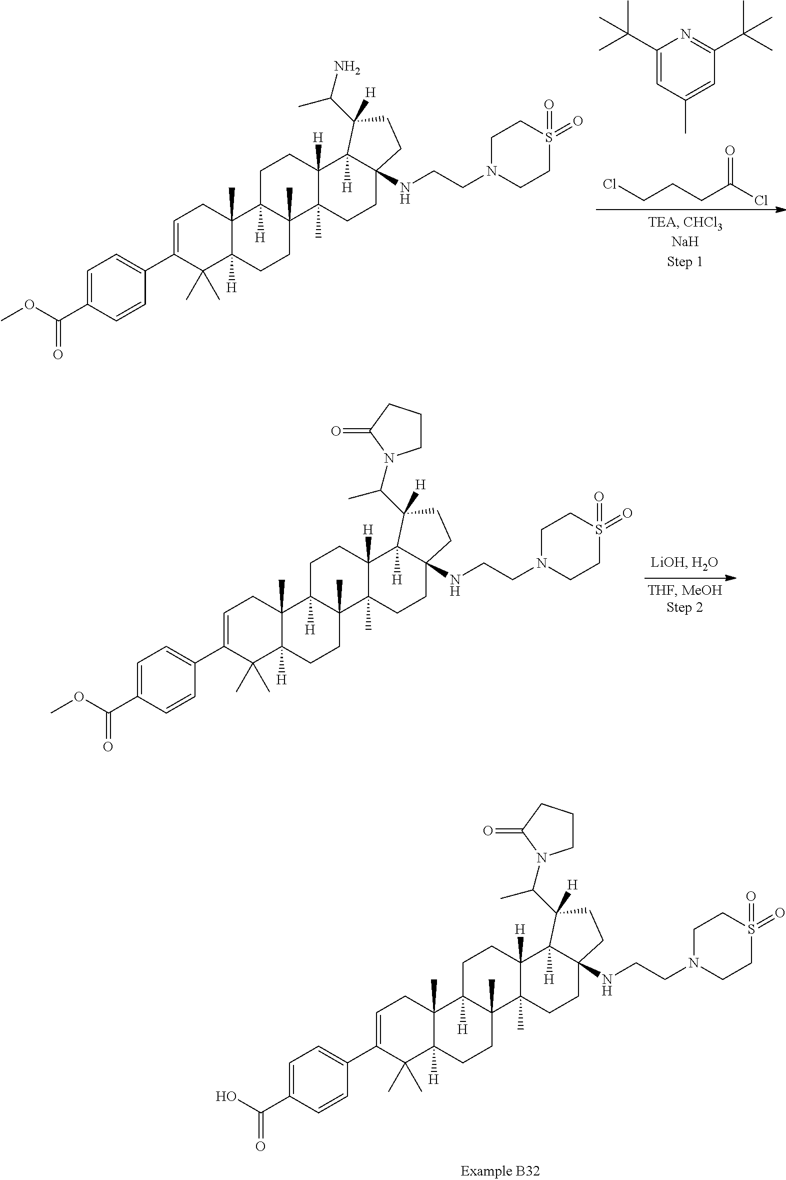

### Step 1. Preparation of methyl 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(1-(2-oxopyrrolidin-1-yl)ethyl)-2,3,3a,4,5,5a,5b,6,7,7a,8,11,11a,11b,12,13,13a,13b-octadecahydro-1H-cyclopenta[a]chrysen-9-yl)benzoate.

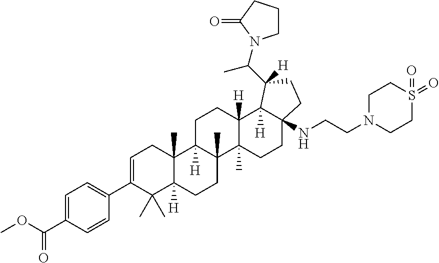

In a 5 mL microwave vial were combined methyl 4-((1R,3aS,5aR,5bR,7aR,11aS,11bR,13aR,13bS)-1-(1-aminoethyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (0.025 g, 0.035 mmol) with 4-chlorobutanoyl chloride (5.94 μl, 0.053 mmol) and 2,6-di-tert-butyl-4-methylpyridine (0.029 g, 0.141 mmol) in a mixture of acetonitrile (0.5 mL) and 1,4-dioxane (0.5 mL). The mixture was stirred at rt for 5 min and was then heated to 120° C. in a microwave reactor for 1 h. The mixture was allowed to cool to rt, then sodium hydride 60% NaH dispersion in mineral oil (excess, approx 20 mg) was added to the mixture causing significant outgassing. The mixture was stirred at rt for 24 h. The crude mixture was carried forward to the next step without further manipulation. LCMS: m/z=776.5 (M+H)⁺, 2.18 min (method 5).

Step 2: The crude reaction mixture containing methyl 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(1-(2-oxopyrrolidin-1-yl)ethyl)-2,3,3a,4,5,5a,5b,6,7,7a,8,11,11a,11b,12,13,13a,13b-octadecahydro-1H-cyclopenta[a]chrysen-9-yl)benzoate (0.027 g, 0.035 mmol) and excess sodium hydride in THF was quenched by slow addition of water (1 mL), resulting in energetic outgassing. Methanol (1 mL) was then added, and the resulting mixture was heated to 60° C. for 30 min. The mixture was concentrated via nitrogen stream to approximately 0.5 mL, diluted with acetonitrile (1 mL) and methanol (1 mL) then filtered and purified by reverse phase preparative HPLC (Prep HPLC Method 3) to afford 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(1-(2-oxopyrrolidin-1-yl)ethyl)-2,3,3a,4,5,5a,5b,6,7,7a,8,11,11a,11b,12,13,13a,13b-octadecahydro-1H-cyclopenta[a]chrysen-9-yl)benzoic acid as a white powder TFA salt (0.0270 g, 76% yield over 2 steps). LCMS: m/z=762.5 (M+H)⁺, 1.98 min (method 5). ¹H NMR (400 MHz, 1:1 mixture of chloroform-d and methanol-d₄, methanol-d₄ lock) δ 7.93 (d, J=8.3 Hz, 2H), 7.21 (d, J=8.3 Hz, 2H), 5.31 (d, J=4.9 Hz, 1H), 4.38-4.28 (m, 1H), 3.62-3.53 (m, 1H), 3.49-3.40 (m, 1H), 3.27-2.93 (m, 11H), 2.49-2.29 (m, 2H), 2.29-2.12 (m, 2H), 1.98 (br. s., 4H), 1.95-1.79 (m, 5H), 1.78-1.68 (m, 2H), 1.52 (d, J=6.1 Hz, 6H), 1.50-1.32 (m, 5H), 1.31-1.25 (m, 2H), 1.22 (br. s., 3H), 1.21 (s, 3H), 1.05 (s, 6H), 0.96 (br. s., 3H), 0.95 (br. s., 3H).

### Example B33

### Preparation of 4-((1R,3aR,5aR,5bR,7aR,11aS,11bR,13aR,13bS)-1-amino-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid.

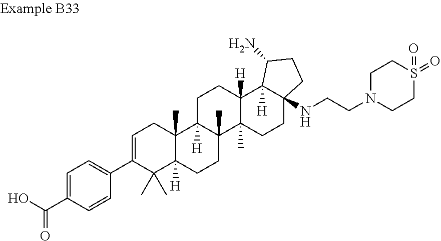

In a 20 mL scintillation vial were combined (1R,3aS,5aR,5bR,7aR,11aS,11bR,13aR,13bS)-3a-((2-(1,1-dioxidothiomorpholino)ethyl)amino)-9-(4-(methoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-1-carboxylic acid, triethylammonium salt (0.100 g, 0.123 mmol) with diphenylphosphoryl azide (0.080 mL, 0.370 mmol) and triethylamine (0.034 mL, 0.247 mmol) in dry 1,4-dioxane (5 mL). The vial was sealed with a PTFE lined screwcap and the mixture was heated to 80° C. for 2 h. The mixture was allowed to cool to rt and stood at rt for 1.5 h. To the rapidly stirred mixture was added sodium hydroxide, 1.0M aqueous (4.93 mL, 4.93 mmol) all at once. The resulting cloudy mixture was stirred rapidly at rt for 30 min. The mixture was concentrated via nitrogen stream and purified by reverse phase preparative HPLC (Prep HPLC Method 15). The desired product was thus isolated as a white solid TFA salt (0.0739 g, 59.5% yield). LCMS: m/z=666.3 (M+H)⁺, 1.79 min (method 5). ¹H NMR (400 MHz, 1:1 mixture of chloroform-d and methanol-d₄, methanol-d₄ lock) δ 7.93 (d, J=8.3 Hz, 2H), 7.21 (d, J=8.3 Hz, 2H), 5.31 (d, J=4.9 Hz, 1H), 3.85 (t, J=8.6 Hz, 1H), 3.27-3.12 (m, 6H), 3.11-2.83 (m, 6H), 2.48 (br. s., 1H), 2.23-1.97 (m, 5H), 1.93-1.80 (m, 2H), 1.75 (d, J=16.1 Hz, 2H), 1.69-1.34 (m, 11H), 1.33-1.24 (m, 2H), 1.21 (s, 3H), 1.09 (s, 3H), 1.06 (s, 3H), 0.97 (s, 3H), 0.96 (s, 3H).

### Example B34

### Preparation of 4-((1R,3aR,5aR,5bR,7aR,11aS,11bR,13aR,13bS)-1-(dimethylamino)-3a-((2-((2-((2-methoxyethyl)sulfonypethyl)(methyl)amino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid.

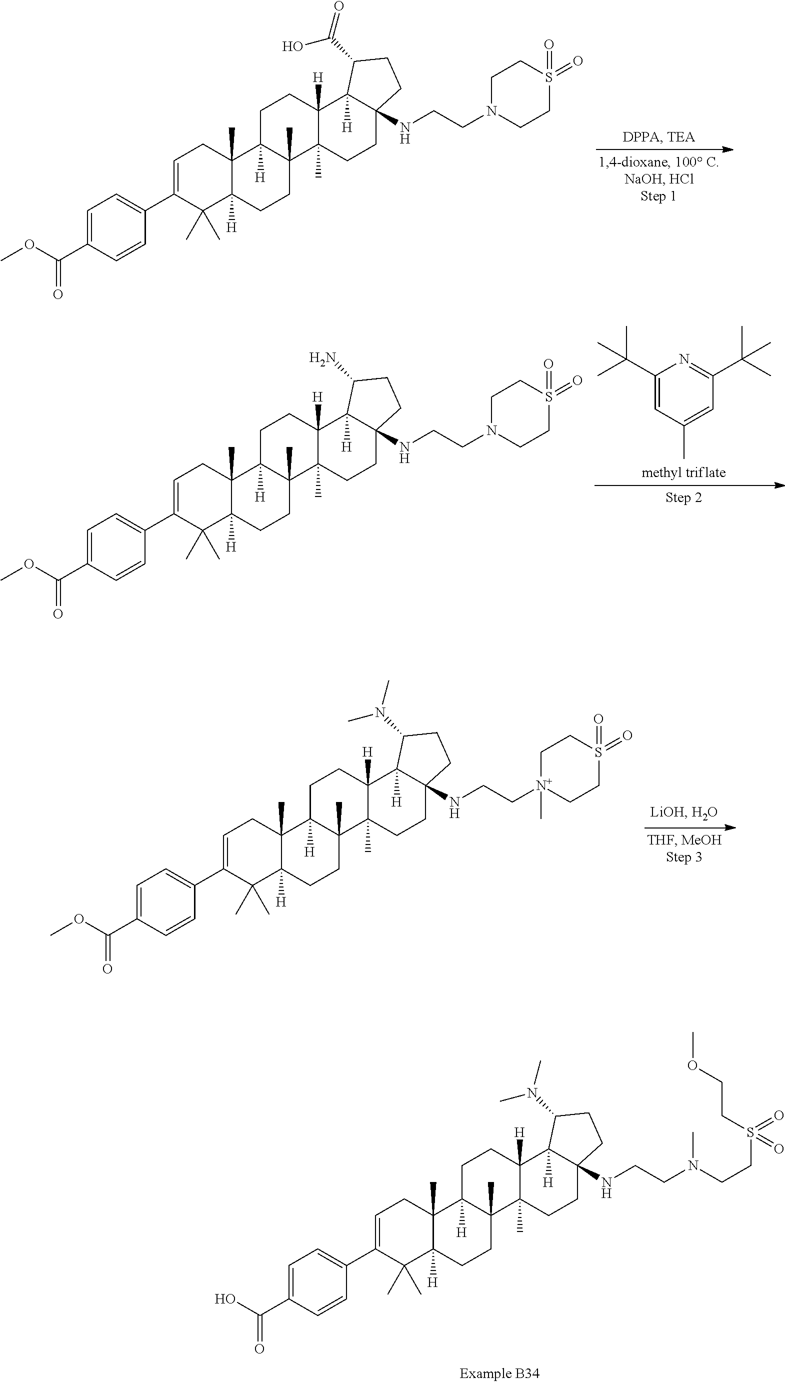

### Step 1. Preparation of methyl 4-((1R,3aR,5aR,5bR,7aR,11aS,11bR,13aR,13bS)-1-amino-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate

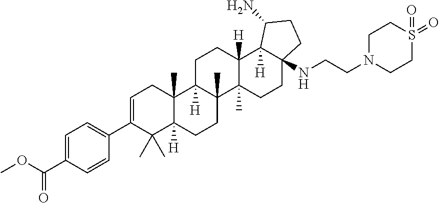

In a 1 dram vial were combined (1R,3aS,5aR,5bR,7aR,11aS,11bR,13aR,13bS)-3a-((2-(1,1-dioxidothiomorpholino)ethyl)amino)-9-(4-(methoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-1-carboxylic acid (0.150 g, 0.212 mmol) with diphenylphosphoryl azide (0.114 mL, 0.529 mmol) and triethylamine (0.118 mL, 0.846 mmol) in dry 1,4-dioxane (2 mL). The vial was sealed with a PTFE lined screwcap and the mixture was heated to 80° C. for 45 min. Additional diphenylphosphoryl azide (0.057 mL, 0.265 mmol) was added and the mixture was heated to 85° C. for another 20 minutes. The mixture was removed from heat, allowed to cool to rt, and to it was added sodium hydroxide, 3.0M aqueous (1.41 mL, 4.23 mmol). After 45 min of stirring, the mixture was cooled in an ice bath and to it was added hydrochloric acid, 12M (0.353 mL, 4.23 mmol) slowly. The crude mixture was purified by reverse phase preparative HPLC (Prep HPLC Method 3). The desired product was isolated as a white powder TFA salt (0.0558 g, 25.8% yield). LCMS: m/z=680.4 (M+H)⁺, 1.93 min (method 5). ¹H NMR (400 MHz, 1:1 mixture of chloroform-d and methanol-d₄, methanol-d₄ lock) δ 7.92 (d, J=8.3 Hz, 2H), 7.22 (d, J=8.3 Hz, 2H), 5.30 (d, J=4.6 Hz, 1H), 3.95-3.81 (m, 4H), 3.30-2.84 (m, 12H), 2.59-2.37 (m, 1H), 2.32-2.21 (m, 1H), 2.21-2.01 (m, 4H), 2.00-1.70 (m, 4H), 1.69-1.33 (m, 12H), 1.33-1.24 (m, 2H), 1.21 (s, 3H), 1.09 (s, 3H), 1.05 (s, 3H), 0.96 (s, 3H), 0.95 (s, 3H).

### Step 2. Preparation of 4-(2-(((1R,3aR,5aR,5bR,7aR,11aS,11bR,13aR,13bS)-1-(dimethylamino)-9-(4-(methoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-3a-yl)amino)ethyl)-4-methylthiomorpholin-4-ium 1,1-dioxide.

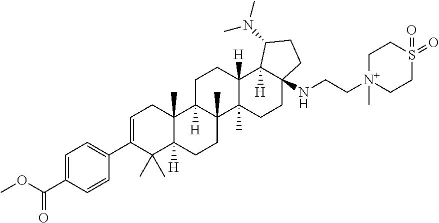

In a 1 dram vial were combined methyl 4-((1R,3aR,5aR,5bR,7aR,11aS,11bR,13aR,13bS)-1-amino-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate TFA salt (0.021 g, 0.021 mmol) with 2,6-di-tert-butyl-4-methylpyridine (0.042 g, 0.205 mmol) in chloroform (0.5 mL). To this stirred mixture was added methyl trifluoromethanesulfonate (0.011 mL, 0.103 mmol). The vial was sealed with a PTFE lined screwcap and the mixture was stirred at 70° C. for 30 min. Another 5 equivalents of methyl trifluoromethanesulfonate (0.011 mL, 0.103 mmol) were added and the mixture was reheated to 70° C. for an additional 105 min. The mixture was concentrated via nitrogen stream and redissolved in minimum methanol with a little THF added. The crude mixture was purified by reverse phase preparative HPLC (Prep HPLC Method 2). The product thus isolated was carried directly into the next step. LCMS: m/z=722.4 (M+H)⁺, 2.04 min (method 5).

Step 3: In a 1 dram vial were combined 4-(2-(((1R,3aR,5aR,5bR,7aR,11aS,11bR,13aR,13bS)-1-(dimethylamino)-9-(4-(methoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-3a-yl)amino)ethyl)-4-methylthiomorpholin-4-ium 1,1-dioxide (0.010 g, 9.12 μmol) with 1.0M aqueous lithium hydroxide hydrate (0.091 mL, 0.091 mmol) and tetrahydrofuran (0.3 mL) and MeOH (0.3 mL). The vial was sealed with a PTFE lined screwcap and the mixture was heated to 70° C. with stirring for 30 min. The crude mixture was purified by reverse phase preparative HPLC (Prep HPLC Method 3) to afford 4-((1R,3aR,5aR,5bR,7aR,11aS,11bR,13aR,13bS)-1-(dimethylamino)-3a-((2-((2-((2-methoxyethyl)sulfonyl)ethyl)(methyl)amino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid as a white powder TFA salt (0.0048 g, 20% yield over 2 steps). LCMS: m/z=740.4 (M+H)⁺, 1.88 min (method 5). ¹H NMR (400 MHz, 1:1 mixture of chloroform-d and methanol-d₄, methanol-d₄ lock) δ 7.93 (d, J=8.1 Hz, 2H), 7.20 (d, J=8.3 Hz, 2H), 5.30 (dd, J=6.0, 1.3 Hz, 1H), 4.24-4.10 (m, 1H), 3.84 (t, J=4.6 Hz, 2H), 3.67-3.56 (m, 2H), 3.49-3.43 (m, J=6.2, 6.2Hz, 2H), 3.25-3.12 (m, 3H), 3.01-2.74 (m, 8H), 2.72 (s, 3H), 2.35-2.21 (m, 1H), 2.21-2.06 (m, 2H), 2.06-1.97 (m, 2H), 1.95-1.85 (m, 2H), 1.73 (d, J=17.1 Hz, 1H), 1.68-1.42 (m, 12H), 1.35-1.20 (m, 8H), 1.17 (s, 3H), 1.07 (s, 3H), 1.05 (s, 3H), 0.96 (br. s., 3H), 0.95 (br. s., 3H).

### Example B35

### Preparation of 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(1-(methylamino)ethyl)-2,3,3a,4,5,5a,5b,6,7,7a,8,11,11a,11b,12,13,13a,13b-octadecahydro-1H-cyclopenta[a]chrysen-9-yl)benzoic acid.

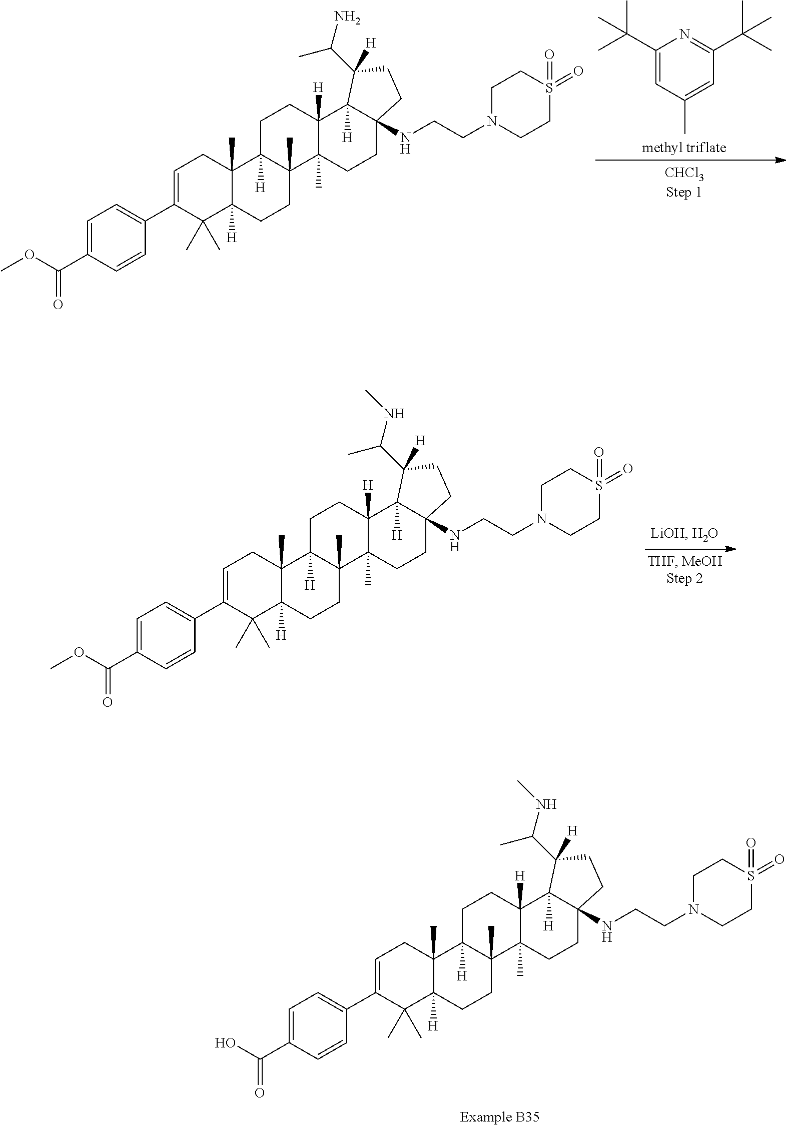

Step 1. Preparation of methyl 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(1-(methylamino)ethyl)-2,3,3a,4,5,5a,5b,6,7,7a,8,11,11a,11b,12,13,13a,13b-octadecahydro-1H-cyclopenta[a]chrysen-9-yl)benzoate.

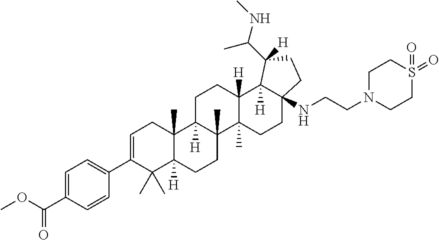

In a 1 dram vial were combined methyl 4-((1R,3aS,5aR,5bR,7aR,11aS,11bR,13aR,13bS)-1-(1-aminoethyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (0.020 g, 0.028 mmol) with 2,6-di-tert-butyl-4-methylpyridine (0.029 g, 0.141 mmol) in chloroform (0.5 mL). To this stirred mixture was added methyl trifluoromethanesulfonate (4.98 μl, 0.045 mmol) and a PTFE lined screwcap was installed and the mixture was stirred at rt for 45 min. To the mixture was added more methyl trifluoromethanesulfonate (7 μl, approx 0.063 mmol, 2.25 equivalents) and stirred at rt for 1.5 h. To the mixture was added 1.0M ammonia in THF, then the mixture was concentrated under nitrogen stream to a residue, redissolved in a minimum amount of a 1:1 THF/MeOH mixture and purified by reverse phase preparative HPLC (Prep HPLC Method 12). Fractions containing the desired product were combined and repurified by reverse phase preparative HPLC (Prep HPLC Method 3) to provide the desired product as a white powder TFA salt (0.0123 g, 40.9% yield). LCMS: m/z=722.3 (M+H)⁺, 2.01 min (method 5). ¹H NMR (400 MHz, 1:1 mixture of chloroform-d and methanol-d₄, methanol-d₄ lock) δ 7.92 (d, J=8.3 Hz, 2H), 7.22 (d, J=8.3 Hz, 2H), 5.31 (d, J=4.4 Hz, 1H), 3.91 (s, 3H), 3.28-2.96 (m, 12H), 2.74 (s, 3H), 2.50 (td, J=10.9, 2.6 Hz, 1H), 2.21-1.91 (m, 6H), 1.90-1.68 (m, 4H), 1.68-1.37 (m, 11H), 1.34 (s, 3H), 1.31-1.24 (m, 2H), 1.21 (s, 3H), 1.12 (s, 3H), 1.05 (s, 3H), 0.96 (br. s., 3H), 0.95 (br. s., 3H).

Step 2: In a 1 dram vial were combined methyl 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(1-(methylamino)ethyl)-2,3,3a,4,5,5a,5b,6,7,7a,8,11,11a,11b,12,13,13a,13b-octadecahydro-1H-cyclopenta[a]chrysen-9-yl)benzoate TFA salt (0.0093 g, 8.74 μmol) with 1.0M aqueous lithium hydroxide hydrate (0.087 mL, 0.087 mmol), tetrahydrofuran (0.3 mL) and MeOH (0.3 mL). The vial was sealed with a PTFE lined screwcap and the mixture was heated to 70° C. with stirring for 30 min. The crude mixture was purified by reverse phase preparative HPLC (Prep HPLC Method 16) to provide 4-((1R,3aS,5aR,5bR,7aR,11aS,11bR,13aR,13bS)-3a-((2-(1,1-dioxidothiomorpholino)ethyl)amino)-5a,5b,8,8,11a-pentamethyl-1-(1-(methylamino)ethyl)-2,3,3a,4,5,5a,5b,6,7,7a,8,11,11a,11b,12,13,13a,13b-octadecahydro-1H-cyclopenta[a]chrysen-9-yl)benzoic acid as a white glassy solid TFA salt (0.0078 g, 80% yield). LCMS: m/z=708.4 (M+H)⁺, 1.79 min (method 5). ¹H NMR (400 MHz, 1:1 mixture of chloroform-d and methanol-d₄, methanol-d₄ lock) δ 7.93 (d, J=8.3 Hz, 2H), 7.21 (d, J=8.3 Hz, 2H), 5.31 (d, J=4.6 Hz, 1H), 3.27-2.95 (m, 12H), 2.74 (s, 3H), 2.57-2.44 (m, 1H), 2.23-1.92 (m, 6H), 1.90-1.68 (m, 4H), 1.68-1.40 (m, 11H), 1.34 (d, J=6.6 Hz, 3H), 1.31-1.24 (m, 3H), 1.21 (s, 3H), 1.12 (s, 3H), 1.05 (s, 3H), 0.97 (br. s., 3H), 0.96 (br. s., 3H).

### Example B36

### Preparation of 4-((1R,3aR,5aR,5bR,7aR,11aS,11bR,13aR,13bS)-1-acetamido-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid.

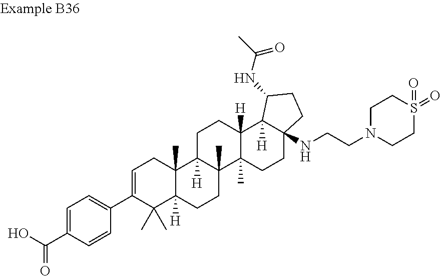

In a 1 dram vial were combined (1R,3aS,5aR,5bR,7aR,11aS,11bR,13aR,13bS)-3a-((2-(1,1-dioxidothiomorpholino)ethyl)amino)-9-(4-(methoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-1-carboxylic acid (0.020 g, 0.028 mmol) with diphenylphosphoryl azide (0.015 mL, 0.071 mmol) and triethylamine (0.016 mL, 0.113 mmol) in dry 1,4-dioxane (1 mL). The vial was sealed with a PTFE lined screwcap and the mixture was heated to 80 degrees C. for 20 min. The reaction mixture was then added via pipette directly to a 80 degree C. vial containing sodium hydroxide, 3.0M aqueous (0.235 mL, 0.705 mmol). The mixture was removed from heat after 1 minute. The mixture was treated with acetic anhydride (0.135 g, 1.33 mmol) and the resulting mixture was heated to 70 degrees C. for 10 min. The crude mixture was purified by reverse phase preparative HPLC (Prep HPLC Method 16) to provide the desired product as a colorless solid TFA salt (0.0149 g, 54% yield). LCMS: m/z=708.3 (M+H)⁺, 1.92 min (method 5). ¹H NMR (400 MHz, 1:1 mixture of chloroform-d and methanol-d₄, methanol-d₄ lock) δ 8.08 (d, J=8.3 Hz, 1H), 7.93 (d, J=8.3 Hz, 2H), 7.20 (d, J=8.3 Hz, 2H), 5.30 (dd, J=6.0, 1.3 Hz, 1H), 4.25 (q, J=11.2Hz, 1H), 3.27-3.00 (m, 11H), 2.91 (dt, J=14.6, 7.2Hz, 1H), 2.38-2.23 (m, 1H), 2.22-2.05 (m, 3H), 2.05-1.96 (m, 1H), 1.92 (s, 3H), 1.90-1.66 (m, 5H), 1.65-1.35 (m, 11H), 1.34-1.22 (m, 3H), 1.18 (s, 3H), 1.09 (s, 3H), 1.04 (s, 3H), 0.96 (br. s., 3H), 0.95 (br. s., 3H).

### Example B37 and Example B38

### Preparation of 4-((1R,3aR,5aR,5bR,7aR,11aS,11bR,13aR,13bS)-3a-((2-(1,1-dioxidothiomorpholino)ethyl)amino)-5a,5b,8,8,11a-pentamethyl-1-(methylamino)-2,3,3a,4,5,5a,5b,6,7,7a,8,11,11a,11b,12,13,13a,13b-octadecahydro-1H-cyclopenta[a]chrysen-9-yl)benzoic acid and 4-((1R,3aR,5aR,5bR,7aR,11aS,11bR,13aR,13bS)-1-(dimethylamino)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid.

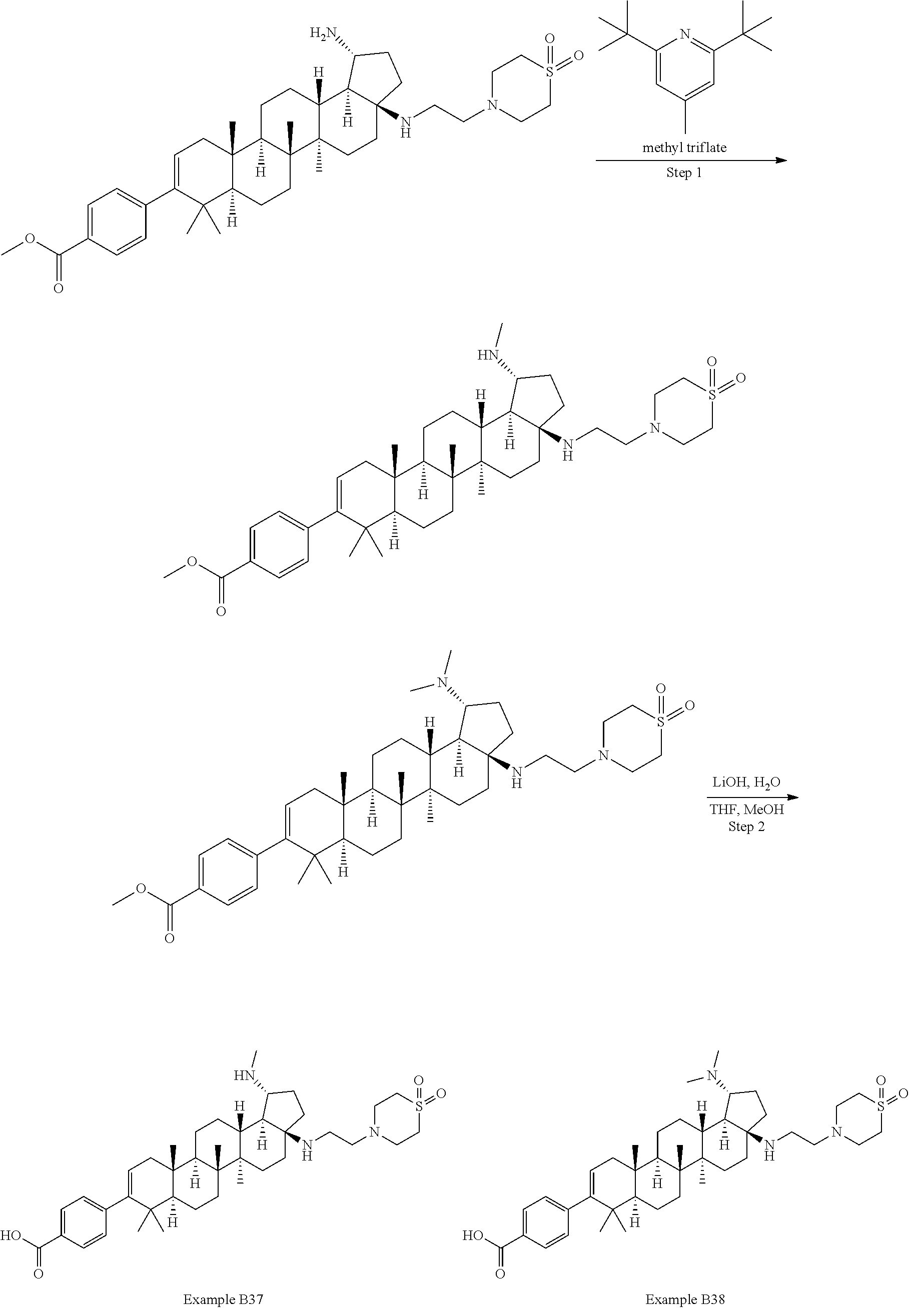

### Step 1. Preparation of methyl 4-((1R,3aR,5aR,5bR,7aR,11aS,11bR,13aR,13bS)-3a-((2-(1,1-dioxidothiomorpholino)ethyl)amino)-5a,5b,8,8,11a-pentamethyl-1-(methylamino)-2,3,3a,4,5,5a,5b,6,7,7a,8,11,11a,11b,12,13,13a,13b-octadecahydro-1H-cyclopenta[a]chrysen-9-yl)benzoate and methyl 4-((1R,3aR,5aR,5bR,7aR,11aS,11bR,13aR,13bS)-1-(dimethylamino)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate.

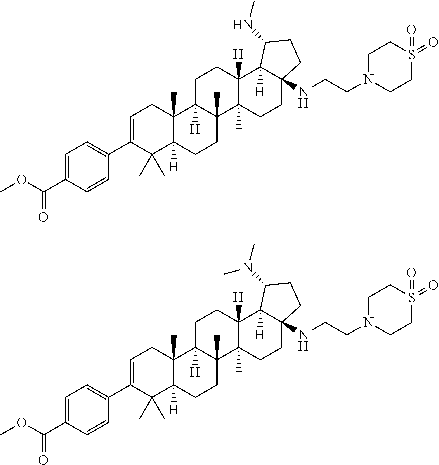

Two separate Step 1 and Step 2 experiments were performed. The final resulting crude after Step 2 reaction mixtures from both experiments were combined and purified to provide the title compounds:

Experiment 1:

In a 1 dram vial were combined methyl 4-((1R,3aR,5aR,5bR,7aR,11aS,11bR,13aR,13bS)-1-amino-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate TFA salt (0.021 g, 0.021 mmol) with 2,6-di-tert-butyl-4-methylpyridine (0.042 g, 0.205 mmol) in chloroform (0.5 mL). To this stirred mixture was added methyl trifluoromethanesulfonate (0.011 mL, 0.103 mmol). A PTFE lined screwcap was installed and the mixture was stirred at rt overnight. The crude mixture was purified by reverse phase preparative HPLC (Prep HPLC Method 16). A mixture of the two desired products was thus obtained as TFA salts (0.0123 g) from prep HPLC. The material was carried directly into the next step (Step 2 Experiment 1). LCMS: m/z=694.6 (M+H)⁺, 1.92 min and 708.6 (M+H)⁺, 2.07 min (method 3).

Experiment 2:

In a 1 dram vial were combined methyl 4-((1R,3aR,5aR,5bR,7aR,11aS,11bR,13aR,13b5)-1-amino-3a-((2-(1,1-25⁵ dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate TFA salt (0.021 g, 0.021 mmol) with 2,6-di-tert-butyl-4-methylpyridine (0.042 g, 0.205 mmol) in chloroform (0.5 mL). To this stirred mixture was added methyl trifluoromethanesulfonate (0.011 mL, 0.103 mmol). A PTFE lined screwcap was installed and the mixture was heated to 70° C. for 30 min. Additional methyl trifluoromethanesulfonate (0.011 mL, 0.103 mmol) was added and the mixture was heated to 70° C. for 105 min. The crude mixture was purified by reverse phase preparative HPLC (Prep HPLC Method 2). A mixture of the two desired products was thus obtained as TFA salts (0.010 g) from prep HPLC. The material was carried directly into the next step (Step 2 Experiment 2).

Step 2:

Experiment 1:

In a 1 dram vial were combined the mixture from Step 1, Experiment 1 containing methyl 4-((1R,3aR,5aR,5bR,7aR,11aS,11bR,13aR,13bS)-3a-((2-(1,1-dioxidothiomorpholino)ethyl)amino)-5a,5b,8,8,11a-pentamethyl-1-(methylamino)-2,3,3a,4,5,5a,5b,6,7,7a,8,11,11a,11b,12,13,13a,13b-octadecahydro-1H-cyclopenta[a]chrysen-9-yl)benzoate TFA salt and methyl 4-((1R,3aR,5aR,5bR,7aR,11aS,11bR,13aR,13b5)-1-(dimethylamino)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate TFA salt (0.0123 g, 0.012 mmol) with 1M aqueous lithium hydroxide hydrate (0.117 mL, 0.117 mmol), tetrahydrofuran (0.3 mL) and MeOH (0.3 mL). The vial was sealed with a PTFE lined screwcap and the mixture was heated to 70° C. with stirring for 30 min. The crude mixture was purified by reverse phase preparative HPLC (Prep HPLC Method 16).

Experiment 2:

In a 1 dram vial were combined the mixture from Step 1, Experiment 2 containing methyl 4-((1R,3aR,5aR,5bR,7aR,11aS,11bR,13aR,13bS)-3a-((2-(1,1-dioxidothiomorpholino)ethyl)amino)-5a,5b,8,8,11a-pentamethyl-1-(methylamino)-2,3,3a,4,5,5a,5b,6,7,7a,8,11,11a,11b,12,13,13a,13b-octadecahydro-1H-cyclopenta[a]chrysen-9-yl)benzoate TFA salt and methyl 4-((1R,3aR,5aR,5bR,7aR,11aS,11bR,13aR,13b5)-1-(dimethylamino)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate TFA salt (0.010 g, 9.52 μmol) with lithium hydroxide hydrate (0.095 mL, 0.095 mmol), tetrahydrofuran (0.3 mL) and MeOH (0.3 mL). The vial was sealed with a PTFE lined screwcap and the mixture was heated to 70 degrees C. with stirring for 30 min. The crude mixture was purified by reverse phase preparative HPLC (Prep HPLC Method 3).

Repurification of Combined Residues from Step 2 Experiment 1 and Step 2

Experiment 2: The fractions containing product from the prep HPLC purifications from Step 2 Experiment 1 and Step 2 Experiment 2 were combined and repurified by reverse phase preparative HPLC (Prep HPLC Method 17). Concentrated like product fractions were converted to TFA salts by treatment with a mixture of 88% acetonitrile, 10% water, 2% TFA followed by reconcentration in vacuo. Thus were obtained the separated desired products as TFA salts.

4-((1R,3aR,5aR,5bR,7aR,11aS,11bR,13aR,13bS)-3a-((2-(1,1-dioxidothiomorpholino)ethyl)amino)-5a,5b,8,8,11a-pentamethyl-1-(methylamino)-2,3,3a,4,5,5a,5b,6,7,7a,8,11,11a,11b,12,13,13a,13b-octadecahydro-1H-cyclopenta[a]chrysen-9-yl)benzoic acid (Example B37): This material was recovered as a colorless solid TFA salt (0.0086 g, 18% combined yield over 2 steps). LCMS: m/z=680.4 (M+H)⁺, 1.73 min (method 5). ¹H NMR (400 MHz, 1:1 mixture of chloroform-d and methanol-d₄, methanol-d₄ lock) δ 7.93 (d, J=8.3 Hz, 2H), 7.21 (d, J=8.1 Hz, 2H), 5.31 (dd, J=5.6, 1.2Hz, 1H), 3.77-3.66 (m, 1H), 3.25-3.01 (m, 8H), 3.00-2.82 (m, 4H), 2.71 (s, 3H), 2.42-2.26 (m, 1H), 2.23-2.12 (m, 1H), 2.11-1.92 (m, 4H), 1.80-1.70 (m, 2H), 1.70-1.43 (m, 10H), 1.43-1.34 (m, 1H), 1.33-1.23 (m, 4H), 1.20 (s, 3H), 1.08 (s, 3H), 1.06 (s, 3H), 0.97 (s, 3H), 0.96 (br. s., 3H).

4-((1R,3aR,5aR,5bR,7aR,11aS,11bR,13aR,13b5)-1-(dimethylamino)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid (Example B38): This material was recovered as a colorless solid TFA salt (0.0097 g, 21% combined yield over 2 steps). LCMS: m/z=694.4 (M+H)⁺, 1.76 min (method 5). ¹H NMR (400 MHz, 1:1 mixture of chloroform-d and methanol-d₄, methanol-d₄ lock) δ 7.93 (d, J=8.1 Hz, 2H), 7.21 (d, J=8.1 Hz, 2H), 5.37-5.24 (m, 1H), 4.22-4.07 (m, 1H), 3.28-3.03 (m, 8H), 3.03-2.89 (m, 4H), 2.85 (s, 6H), 2.35-2.22 (m, 1H), 2.21-1.99 (m, 6H), 1.81-1.34 (m, 14H), 1.33-1.24 (m, 2H), 1.20 (s, 3H), 1.12 (s, 3H), 1.05 (s, 3H), 0.97 (br. s., 3H), 0.96 (br. s., 3H).

### Example B39

### Preparation of 4-((1R,3aS,5aR,5bR,7aR,11aS,11bR,13aR,13bS)-1-(1-(dimethylamino)ethyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid Isomer 1.

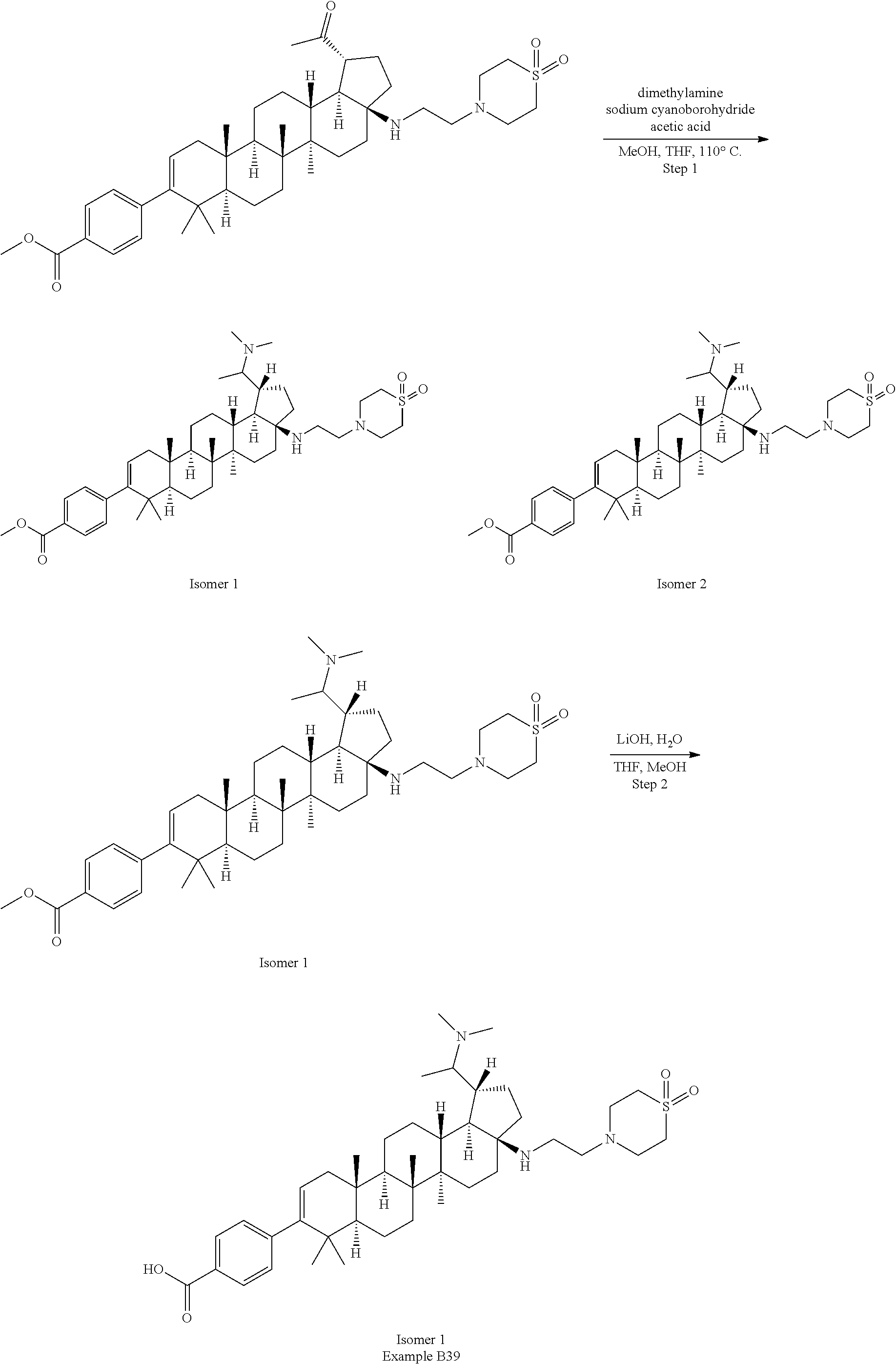

### Step 1. Preparation of methyl 4-((1R,3aS,5aR,5bR,7aR,11aS,11bR,13aR,13bS)-1-(1-(dimethylamino)ethyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 1 and methyl 4-((1R,3aS,5aR,5bR,7aR,11aS,11bR,13aR,13bS)-1-(1-(dimethylamino)ethyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 2.

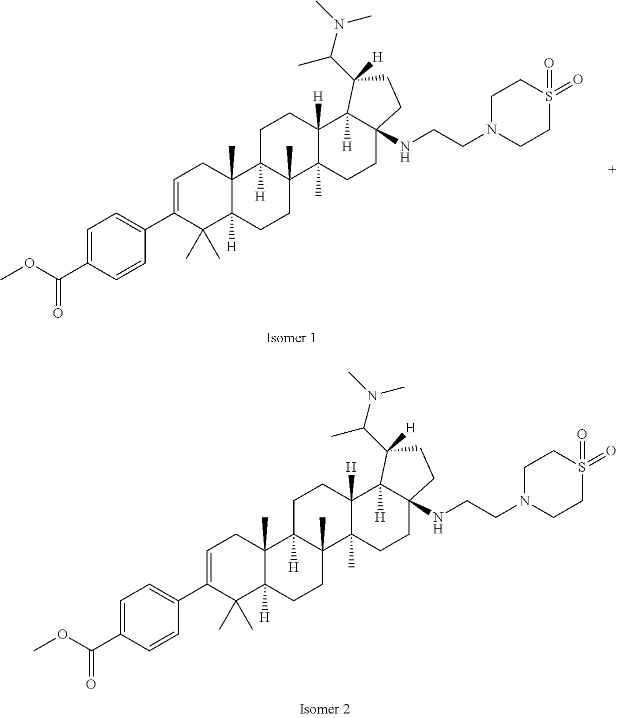

In a 15 mL pressure vessel, a solution of methyl 4-((1R,3aS,5aR,5bR,7aR,11aS,11bR,13aR,13bS)-1-acetyl-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (0.100 g, 0.141 mmol) in MeOH (1 mL) was treated with dimethylamine, 2.0M in THF (1.061 mL, 2.122 mmol), acetic acid (0.121 mL, 2.122 mmol) and sodium cyanoborohydride (0.019 g, 0.283 mmol). The vessel was sealed and the mixture was heated in an oil bath to 110° C. for 6 d. The crude mixture was purified by reverse phase preparative HPLC (Prep HPLC Method 16). The desired products were thus isolated:

Methyl 4-((1R,3aS,5aR,5bR,7aR,11aS,11bR,13aR,13bS)-1-(1-(dimethylamino)ethyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 1 was the major product and was recovered as a white solid TFA salt (0.0246 g, 16.1% yield). LCMS: m/z=736.4 (M+H)⁺, 2.05 min (method 5).

Methyl 4-((1R,3aS,5aR,5bR,7aR,11aS,11bR,13aR,13bS)-1-(1-(dimethylamino)ethyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 2 was the minor product and was recovered as a white sticky solid TFA salt (0.0116 g, 7.6% yield).

Step 2: In a 1 dram vial were combined methyl 4-((1R,3aS,5aR,5bR,7aR,11aS,11bR,13aR,13bS)-1-((R)-1-(dimethylamino)ethyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 1 TFA salt (0.0246 g, 0.023 mmol) with 1M aqueous lithium hydroxide hydrate (0.228 mL, 0.228 mmol), tetrahydrofuran (0.3 mL) and MeOH (0.3 mL). The vial was sealed with a PTFE lined screwcap and the mixture was heated to 80° C. with stirring for 20 min. The crude mixture was purified by reverse phase preparative HPLC (Prep HPLC Method 16). It was necessary to combine the product fractions and repurify by reverse phase preparative HPLC (Prep HPLC Method 15) to afford 4-((1R,3aS,5aR,5bR,7aR,11aS,11bR,13aR,13bS)-1-(1-(dimethylamino)ethyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid Isomer 1 as a white glass solid TFA salt (0.0151 g, 62.2% yield). LCMS: m/z=722.4 (M+H)⁺, 1.80 min (method 5). ¹H NMR (400 MHz, METHANOL-d₄) δ 7.95 (d, J=8.3 Hz, 2H), 7.25 (d, J=8.3 Hz, 2H), 5.34 (d, J=4.6 Hz, 1H), 3.56-3.45 (m, 1H), 3.30-3.05 (m, 10H), 3.04-2.78 (m, 7H), 2.63-2.50 (m, 1H), 2.29-1.97 (m, 6H), 1.93-1.75 (m, 4H), 1.73-1.48 (m, 11H), 1.40 (d, J=6.8 Hz, 3H), 1.38-1.30 (m, 2H), 1.29 (s, 3H), 1.20 (s, 3H), 1.11 (s, 3H), 1.01 (s, 3H), 0.99 (s, 3H).

### Example B40

### Preparation of 4-((1R,3aS,5aR,5bR,7aR,11aS,11bR,13aR,13bS)-1-(1-(dimethylamino)ethyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid Isomer 2.

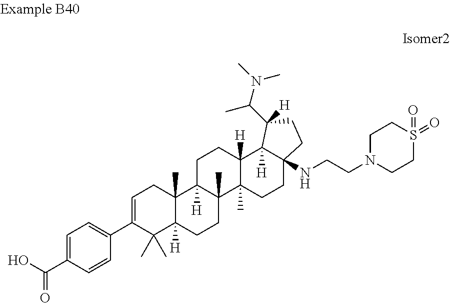

In a 1 dram vial were combined methyl 4-((1R,3aS,5aR,5bR,7aR,11aS,11bR,13aR,13bS)-1-((R)-1-(dimethylamino)ethyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 2 TFA salt (0.0116 g, 10.76 μmol) with 1M aqueous lithium hydroxide hydrate (0.108 mL, 0.108 mmol), tetrahydrofuran (0.3 mL) and MeOH (0.3 mL). The vial was sealed with a PTFE lined screwcap and the mixture was heated to 80° C. with stirring for 20 min. The crude mixture was purified by reverse phase preparative HPLC (Prep HPLC Method 3). The desired product was isolated as a sticky gum TFA salt (0.0053 g, 46.3% yield). LCMS: m/z=722.4 (M+H)⁺, 1.82 min (method 5). ¹H NMR (400 MHz, METHANOL-d₄) δ 7.95 (d, J=8.1 Hz, 2H), 7.25 (d, J=8.1 Hz, 2H), 5.34 (d, J=4.4 Hz, 1H), 3.55 (d, J=7.1 Hz, 1H), 3.29-3.18 (m, 8H), 3.14 (br. s., 4H), 2.91 (br. s., 6H), 2.26-1.98 (m, 6H), 1.95-1.73 (m, 5H), 1.72-1.47 (m, 13H), 1.41 (d, J=6.6 Hz, 4H), 1.35-1.23 (m, 13H), 1.14 (s, 3H), 1.11 (s, 3H), 1.02 (s, 3H), 0.99 (s, 3H), 0.96-0.86 (m, 3H).

### Example B41

### Preparation of 4-((1R,3aS,5aR,5bR,7aR,11aS,11bR,13aR,13bS)-1-(1-aminoethyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid Isomer 2.

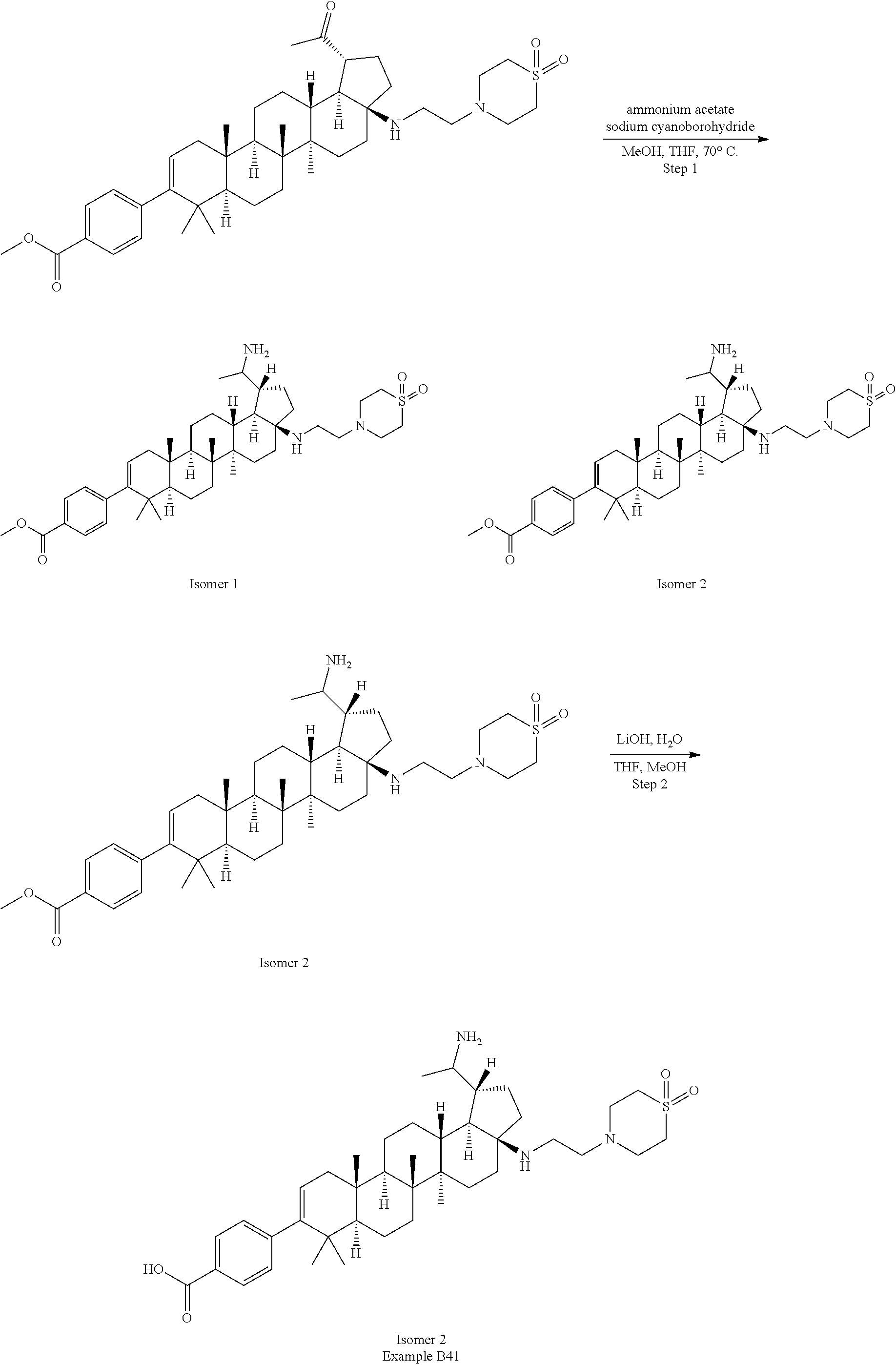

### Step 1. Preparation of methyl 4-((1R,3aS,5aR,5bR,7aR,11aS,11bR,13aR,13bS)-1-(1-aminoethyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 1 and methyl 4-((1R,3aS,5aR,5bR,7aR,11aS,11bR,13aR,13bS)-1-(1-aminoethyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 2.

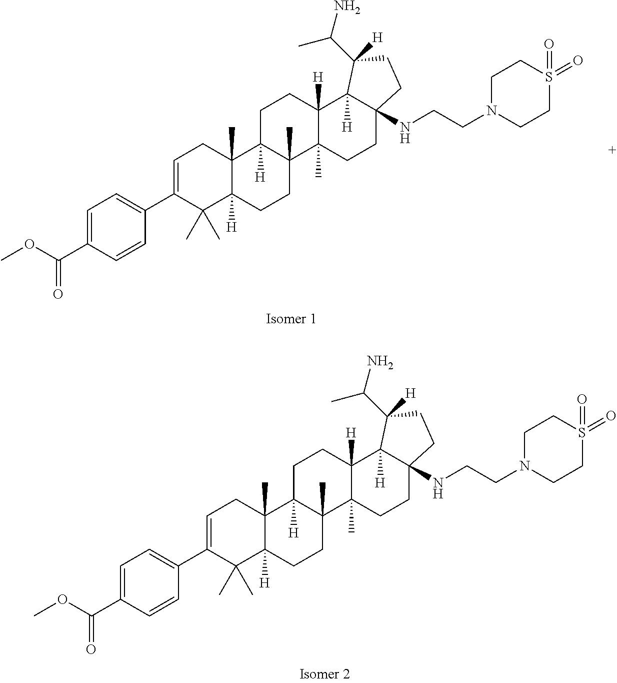

To a solution of methyl 4-((1R,3aS,5aR,5bR,7aR,11aS,11bR,13aR,13bS)-1-acetyl-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (0.050 g, 0.071 mmol) in MeOH (0.5 mL) and THF (0.5 mL) was added ammonium acetate (0.055 g, 0.707 mmol) and sodium cyanoborohydride (7.02 mg, 0.106 mmol). The reaction mixture was stirred at rt for 24 h and was then heated to 70° C. for 6d. The crude mixture was purified by reverse phase preparative HPLC (Prep HPLC Method 16) to provide the desired products: Methyl 4-((1R,3aS,5aR,5bR,7aR,11aS,11bR,13aR,13bS)-1-(1-aminoethyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 1: Isolated as a colorless solid TFA salt (0.0233 g, 31.4% yield). LCMS: m/z=708.4 (M+H)⁺, 2.01 min (method 5). ¹H NMR (400 MHz, 1:1 mixture of chloroform-d and methanol-d₄, methanol-d₄ lock) δ 7.92 (d, J=8.3 Hz, 2H), 7.22 (d, J=8.3 Hz, 2H), 5.30 (d, J=4.6 Hz, 1H), 3.91 (s, 3H), 3.49-3.39 (m, 1H), 3.30-2.92 (m, 12H), 2.44 (dd, J=8.9, 6.2Hz, 1H), 2.16 (dd, J=17.1, 6.4 Hz, 1H), 2.11-1.92 (m, 5H), 1.90-1.77 (m, 2H), 1.73 (d, J=17.1 Hz, 1H), 1.69-1.35 (m, 13H), 1.33-1.24 (m, 5H), 1.21 (s, 3H), 1.11 (s, 3H), 1.05 (s, 3H), 0.96 (s, 3H), 0.95 (s, 3H).

Methyl 4-((1R,3aS,5aR,5bR,7aR,11aS,11bR,13aR,13bS)-1-(1-aminoethyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 2: Isolated as a colorless solid TFA salt (0.0090 g, 12.1% yield). LCMS: m/z=708.4 (M+H)⁺, 2.06 min (method 5). ¹H NMR (400 MHz, 1:1 mixture of chloroform-d and methanol-d₄, methanol-d₄ lock) δ 7.92 (d, J=8.3 Hz, 2H), 7.22 (d, J=8.3 Hz, 2H), 5.30 (d, J=4.6 Hz, 1H), 3.91 (s, 3H), 3.58-3.47 (m, 1H), 3.28-3.12 (m, 8H), 3.12-2.95 (m, 4H), 2.75-2.62 (m, 1H), 2.23-2.05 (m, 3H), 2.05-1.84 (m, 3H), 1.83-1.39 (m, 15H), 1.35-1.23 (m, 7H), 1.21 (s, 3H), 1.09 (s, 3H), 1.05 (s, 3H), 0.96 (s, 3H), 0.95 (br. s., 3H).

### Step 2. Preparation of 4-((1R,3aS,5aR,5bR,7aR,11aS,11bR,13aR,13bS)-1-(1-aminoethyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid Isomer 2

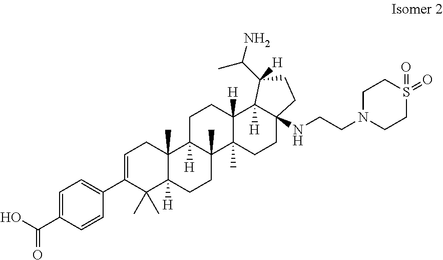

In a 1 dram vial were combined methyl 4-((1R,3aS,5aR,5bR,7aR,11aS,11bR,13aR,13bS)-1-((R)-1-aminoethyl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate Isomer 2 TFA salt (0.0081 g, 7.71 mmol) with 1M aqueous lithium hydroxide hydrate (0.077 mL, 0.077 mmol), tetrahydrofuran (0.3 mL) and MeOH (0.3 mL). The vial was sealed with a PTFE lined screwcap and the mixture was heated to 80° C. with stirring for 20 min. The crude mixture was purified by reverse phase preparative HPLC (Prep HPLC Method 16). The desired compound was isolated as a white glassy solid TFA salt (0.0076 g, 93% yield). LCMS: m/z=694.4 (M+H)⁺, 1.83 min (method 5). ¹H NMR (400 MHz, METHANOL-d₄) δ 7.95 (d, J=8.1 Hz, 2H), 7.25 (d, J=8.1 Hz, 2H), 5.34 (d, J=4.6 Hz, 1H), 3.58 (q, J=6.3 Hz, 1H), 3.30-3.17 (m, 8H), 3.17-3.04 (m, 3H), 2.98 (d, J=5.1 Hz, 1H), 2.66 (br. s., 1H), 2.28-2.10 (m, 3H), 2.09-1.99 (m, 1H), 1.77 (br. s., 5H), 1.75-1.45 (m, 11H), 1.44-1.29 (m, 7H), 1.27 (s, 3H), 1.14 (s, 3H), 1.11 (s, 3H), 1.01 (s, 3H), 0.99 (s, 3H).

### Example B42

### Preparation of 4-((1R,3aS,5aR,5bR,7aR,11aS,11bR,13aR,13bS)-3a-((2-(1,1-dioxidothiomorpholino)ethyl)amino)-1-(hydroxymethyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid.

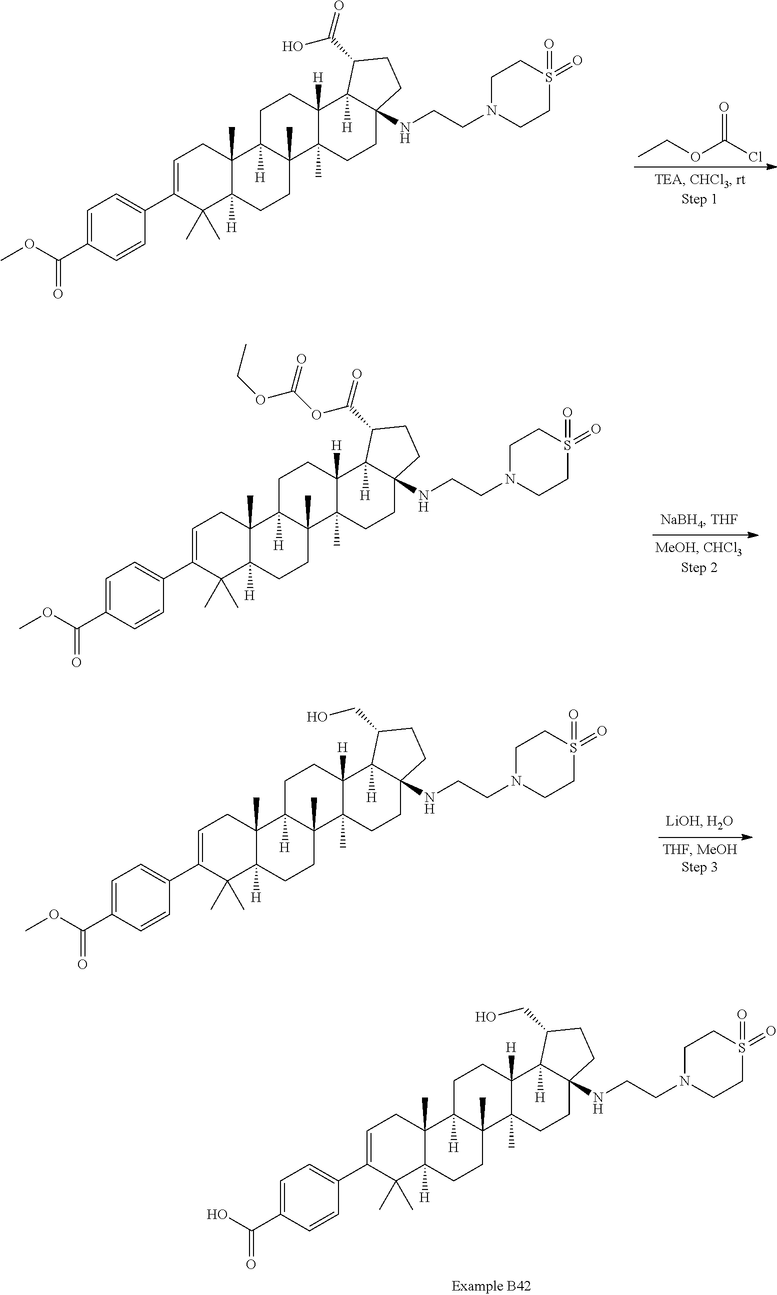

### Step 1. Preparation of (ethyl carbonic) (1R,3aS,5aR,5bR,7aR,11aS,11bR,13aR,13bS)-3a-((2-(1,1-dioxidothiomorpholino)ethyl)amino)-9-(4-(methoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-1-carboxylic anhydride

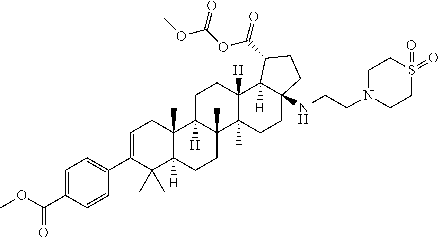

In a 1 dram vial were combined (1R,3aS,5aR,5bR,7aR,11aS,11bR,13aR,13bS)-3a-((2-(1,1-dioxidothiomorpholino)ethyl)amino)-9-(4-(methoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-1-carboxylic acid, triethylammonium salt (0.020 g, 0.025 mmol) with ethyl chloroformate (0.014 ml, 0.148 mmol) and triethylamine (6.87 μl, 0.049 mmol) in dry chloroform. The mixture was stirred at rt for 10 min. The crude reaction mixture was used directly in the next step. LCMS: m/z=781.4 (M+H)⁺, 2.30 min (method 5).

### Step 2. Preparation of methyl 4-((1R,3aS,5aR,5bR,7aR,11aS,11bR,13aR,13bS)-3a-((2-(1,1-dioxidothiomorpholino)ethyl)amino)-1-(hydroxymethyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate.

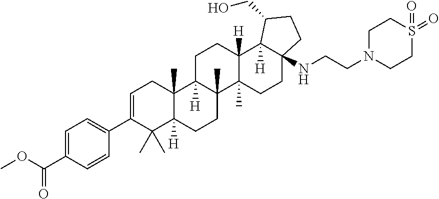

The crude mixture containing (ethyl carbonic) (1R,3aS,5aR,5bR,7aR,11aS,11bR,13aR,13bS)-3a-((2-(1,1-dioxidothiomorpholino)ethyl)amino)-9-(4-(methoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-1-carboxylic anhydride (0.020 g, 0.025 mmol) in chloroform (1 mL) was diluted with THF (0.75 mL) and was treated with sodium borohydride (9.46 mg, 0.250 mmol). After 5 min, no gas evolution was apparent and solid sodium borohydride remained floating in the mixture. To the mixture was added dry methanol (0.75 mL), and effervescence immediately occurred. After 15 min, additional sodium borohydride (9.46 mg, 0.250 mmol) was added and the mixture was stirred overnight under nitrogen. The crude mixture was concentrated under nitrogen stream and was then redissolved in a minimum amount of a mixture of MeOH and THF and was purified by reverse phase preparative HPLC (Prep HPLC Method 12). The desired product was obtained as a white glass solid (0.0080 g, 93% yield). LCMS: m/z=695.4 (M+H)⁺, 2.20 min (method 5).

Step 3: In a 1 dram vial were combined methyl 4-((1R,3aS,5aR,5bR,7aR,11aS,11bR,13aR,13bS)-3a-((2-(1,1-dioxidothiomorpholino)ethyl)amino)-1-(hydroxymethyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (0.008 g, 0.012 mmol) with 1M aqueous lithium hydroxide hydrate (0.115 mL, 0.115 mmol), tetrahydrofuran (0.3 mL) and MeOH (0.3 mL). The vial was sealed with a PTFE lined screwcap and the mixture was heated to 80° C. with stirring for 30 min. The crude mixture was purified by reverse phase preparative HPLC (Prep HPLC Method 18) to afford 4-((1R,3aS,5aR,5bR,7aR,11aS,11bR,13aR,13bS)-3a-((2-(1,1-dioxidothiomorpholino)ethyl)amino)-1-(hydroxymethyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid (0.0055 g, 52.6% yield). LCMS: m/z=681.6 (M+H)⁺, 1.99 min (method 5). ¹H NMR (500 MHz, DMSO-d₆) δ 7.97 (s, 1H), 7.88 (d, J=8.2Hz, 2H), 7.24 (d, J=7.9 Hz, 2H), 5.28 (d, J=5.2Hz, 1H), 4.63 (br. s., 1H), 3.56-3.48 (m, 1H), 3.21-3.03 (m, 12H), 2.13 (dd, J=17.2, 6.6 Hz, 1H), 2.03 (br. s., 2H), 1.84 (d, J=7.0 Hz, 3H), 1.78-1.64 (m, 3H), 1.61-1.39 (m, 10H), 1.34 (d, J=16.2Hz, 1H), 1.30-1.20 (m, 4H), 1.11 (s, 3H), 1.00 (br. s., 3H), 0.98 (br. s., 3H), 0.92 (br. s., 6H).

### Example B43 and Example B44

### Preparation of 4-((1R,3aR,5aR,5bR,7aR,11aS,11bR,13aR,13bS)-3a-((2-(1,1-dioxidothiomorpholino)ethyl)amino)-5a,5b,8,8,11a-pentamethyl-1-(2,2,2-trifluoroacetamido)-2,3,3a,4,5,5a,5b,6,7,7a,8,11,11a,11b,12,13,13a,13b-octadecahydro-1H-cyclopenta[a]chrysen-9-yl)benzoic acid and 4-((1R,3aR,5aR,5bR,7aR,11aS,11bR,13aR,13bS)-3a-((2-(1,1-dioxidothiomorpholino)ethyl)amino)-5a,5b,8,8,11a-pentamethyl-1-(2,2,2-trifluoro-N-methylacetamido)-2,3,3a,4,5,5a,5b,6,7,7a,8,11,11a,11b,12,13,13a,13b-octadecahydro-1H-cyclopenta[a]chrysen-9-yl)benzoic acid

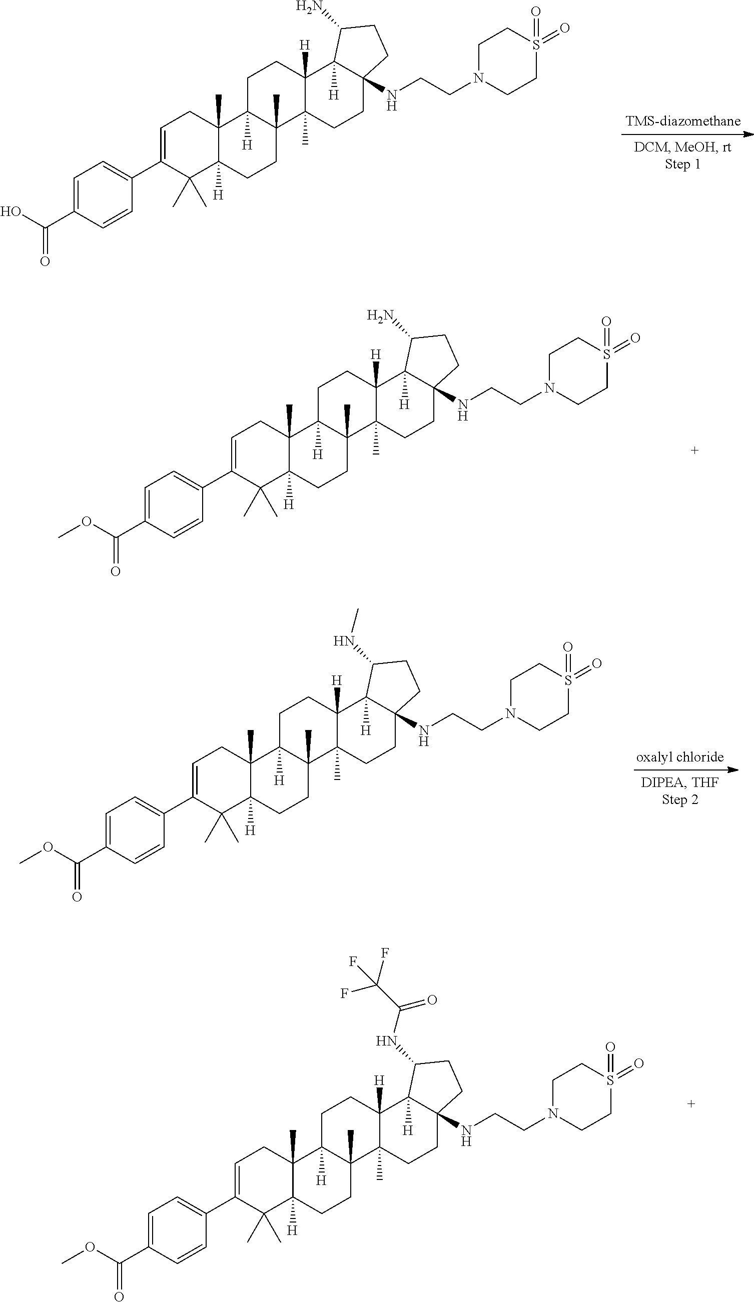

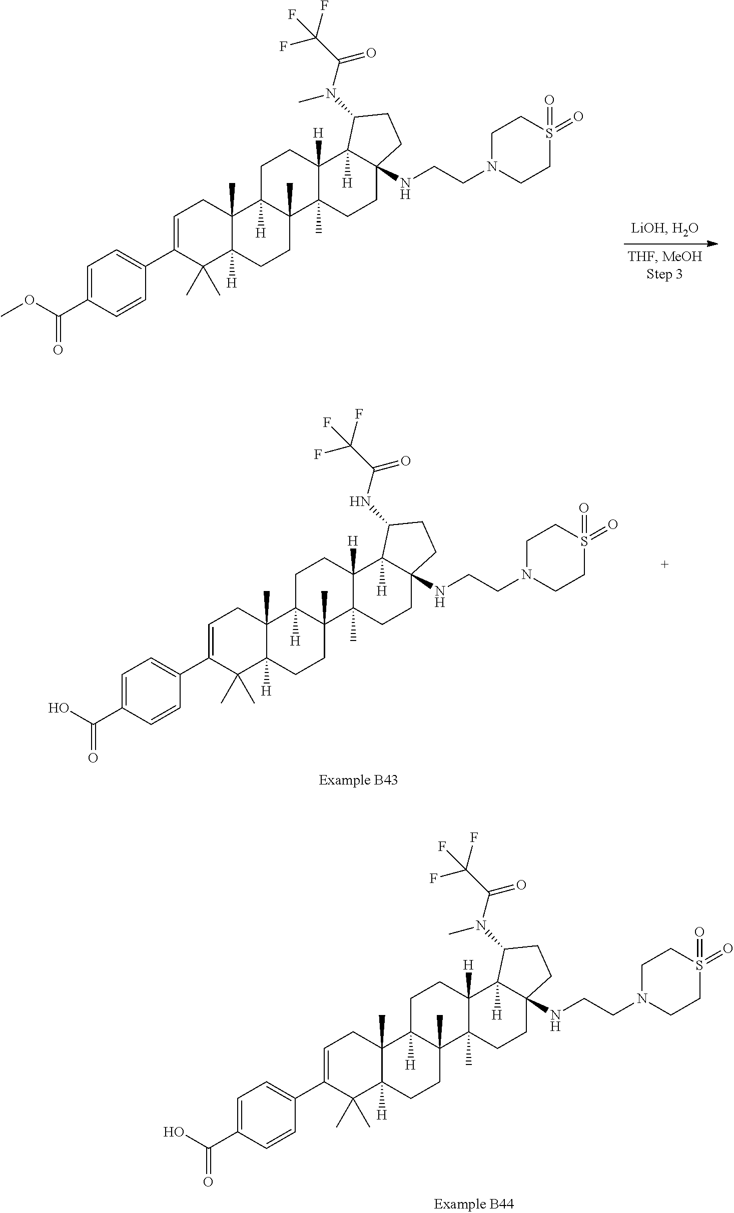

### Step 1. Preparation of methyl 4-((1R,3aR,5aR,5bR,7aR,11aS,11bR,13aR,13bS)-1-amino-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate and methyl 4-((1R,3aR,5aR,5bR,7aR,11aS,11bR,13aR,13bS)-3a-((2-(1,1-dioxidothiomorpholino)ethyl)amino)-5a,5b,8,8,11a-pentamethyl-1-(methylamino)-2,3,3a,4,5,5a,5b,6,7,7a,8,11,11a,11b,12,13,13a,13b-octadecahydro-1H-cyclopenta[a]chrysen-9-yl)benzoate

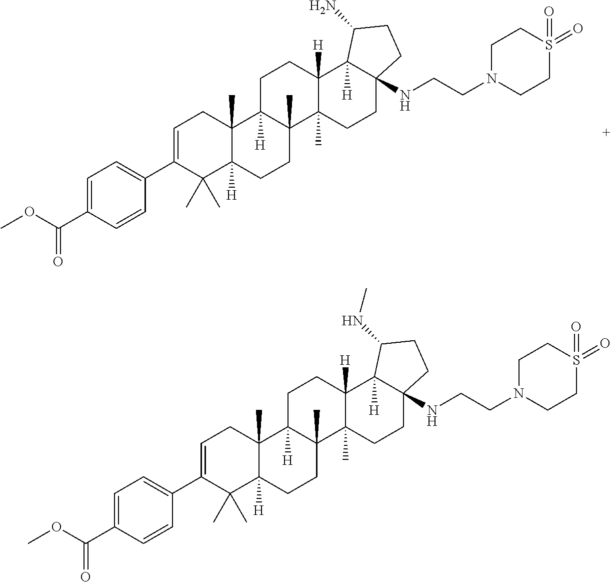

In a 100 mL round bottom flask were combined 4-((1R,3aR,5aR,5bR,7aR,11aS,11bR,13aR,13bS)-1-amino-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid TFA salt (0.410 g, 0.407 mmol) with dry DCM (15 mL) and methanol (15 mL). To the solution was added TMS-Diazomethane (1.424 mL, 2.85 mmol). A slight exotherm and significant outgassing ensued after approximately half of the diazomethane solution had been added. The mixture was stirred at rt for 3 h and was then concentrated in vacuo to a solid residue. The crude mixture was purified by reverse phase preparative HPLC (Prep HPLC Method 4). A mixture of the two desired products was thus obtained as a slightly yellow solid TFA salt (0.259 g, 70.2% yield). The mixture was carried forward directly into the next step.

LCMS: m/z=680.5 (M+H)⁺ and 694.6 (M+H)⁺, 2.04 min (method 5).

### Step 2. Preparation of methyl 4-((1R,3aR,5aR,5bR,7aR,11aS,11bR,13aR,13bS)-3a-((2-(1,1-dioxidothiomorpholino)ethyl)amino)-5a,5b,8,8,11a-pentamethyl-1-(2,2,2-trifluoroacetamido)-2,3,3a,4,5,5a,5b,6,7,7a,8,11,11a,11b,12,13,13a,13b-octadecahydro-1H-cyclopenta[a]chrysen-9-yl)benzoate and methyl 4-((1R,3aR,5aR,5bR,7aR,11aS,11bR,13aR,13bS)-3a-((2-(1,1-dioxidothiomorpholino)ethyl)amino)-5a,5b,8,8,11a-pentamethyl-1-(2,2,2-trifluoro-N-methylacetamido)-2,3,3a,4,5,5a,5b,6,7,7a,8,11,11a,11b,12,13,13a,13b-octadecahydro-1H-cyclopenta[a]chrysen-9-yl)benzoate.

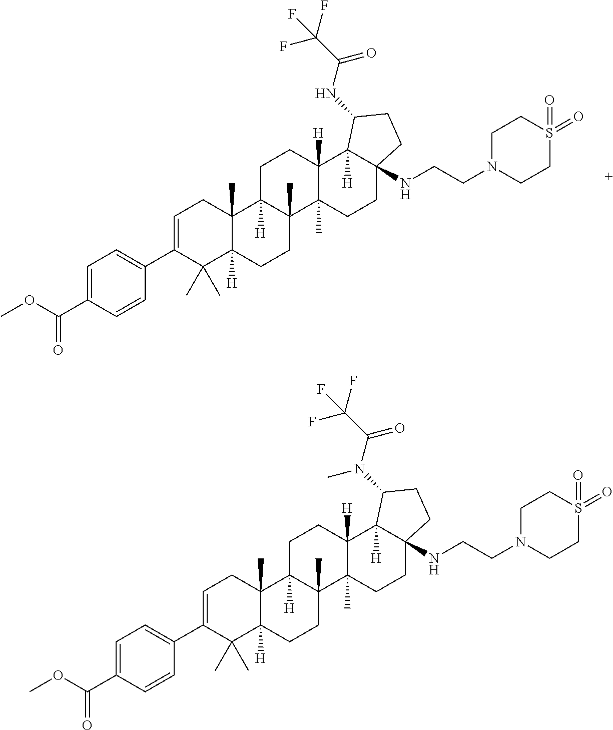

A standard solution of oxalyl dichloride (14.0 mg; 0.110 mmol) in dry THF (1 mL) was prepared. In a 1 dram vial were combined the mixture from Step 1 containing methyl 4-((1R,3aR,5aR,5bR,7aR,11aS,11bR,13aR,13b5)-1-amino-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate TFA salt and methyl 4-((1R,3aR,5aR,5bR,7aR,11aS,11bR,13aR,13bS)-3a-((2-(1,1-dioxidothiomorpholino)ethyl)amino)-5a,5b,8,8,11a-pentamethyl-1-(methylamino)-2,3,3a,4,5,5a,5b,6,7,7a,8,11,11a,11b,12,13,13a,13b-octadecahydro-1H-cyclopenta[a]chrysen-9-yl)benzoate TFA salt (0.020 g, 0.022 mmol) with DIPEA (0.019 mL, 0.110 mmol) in dry THF (0.5 mL). To this mixture was added 0.1 mL of the standard oxalyl dichloride/THF solution which contained oxalyl dichloride (1.396 mg, 0.011 mmol), and the resulting mixture was stirred at rt for 3 h. To the mixture was added another equivalent of oxalyl dichloride (1.396 mg, 0.011 mmol) (0.1 mL of the standard solution). The mixture was stirred for 1 h at rt, and then added another equivalent of oxalyl dichloride (1.396 mg, 0.011 mmol) (0.1 mL of the standard solution) and stirred at rt for 30 min. The mixture was concentrated under a nitrogen stream and the crude residue was carried directly into the next step as-is. LCMS: m/z=776.5 (M+H)⁺, 2.28 min and m/z=790.6 (M+H)⁺, 2.47 min (method 5).

Step 3: In a 1 dram vial, the crude reaction mixture from Step 2 containing methyl 4-((1R,3aR,5aR,5bR,7aR,11aS,11bR,13aR,13bS)-3a-((2-(1,1-dioxidothiomorpholino)ethyl)amino)-5a,5b,8,8,11a-pentamethyl-1-(2,2,2-trifluoroacetamido)-2,3,3a,4,5,5a,5b,6,7,7a,8,11,11a,11b,12,13,13a,13b-octadecahydro-1H-cyclopenta[a]chrysen-9-yl)benzoate and methyl 4-((1R,3aR,5aR,5bR,7aR,11aS,11bR,13aR,13bS)-3a-((2-(1,1-dioxidothiomorpholino)ethyl)amino)-5a,5b,8,8,11a-pentamethyl-1-(2,2,2-trifluoro-N-methylacetamido)-2,3,3a,4,5,5a,5b,6,7,7a,8,11,11a,11b,12,13,13a,13b-octadecahydro-1H-cyclopenta[a]chrysen-9-yl)benzoate (0.017 g, 0.022 mmol) was treated with 1M aqueous lithium hydroxide hydrate (0.176 mL, 0.176 mmol) and MeOH (0.3 mL) was added. The vial was sealed with a PTFE lined screwcap and the mixture was heated to 70° C. with stirring for 30 min. The crude mixture was purified by reverse phase preparative HPLC (Prep HPLC Method 6). Thus was obtained the two desired products.

4-((1R,3aR,5aR,5bR,7aR,11aS,11bR,13aR,13bS)-3a#2-(1,1-dioxidothiomorpholino)ethyl)amino)-5a,5b,8,8,11a-pentamethyl-1-(2,2,2-trifluoroacetamido)-2,3,3a,4,5,5a,5b,6,7,7a,8,11,11a,11b,12,13,13a,13b-octadecahydro-1H-cyclopenta[a]chrysen-9-yl)benzoic acid was the first compound to elute from preparative HPLC. This product was isolated as a white powder TFA salt (0.0110 g, 50% yield). LCMS: m/z=762.4 (M+H)⁺, 2.11 min (method 5). ¹H NMR (400 MHz, 1:1 mixture of chloroform-d and methanol-d₄, methanol-d₄ lock)) δ 7.93 (d, J=8.3 Hz, 2H), 7.20 (d, J=8.1 Hz, 2H), 5.31 (s, 1H), 4.39 (td, J=11.2, 2.8 Hz, 1H), 3.27-2.82 (m, 12H), 2.45-2.28 (m, 2H), 2.21-1.99 (m, 3H), 1.98-1.85 (m, 2H), 1.84-1.70 (m, 3H), 1.67-1.36 (m, 10H), 1.33-1.24 (m, 3H), 1.19 (s, 3H), 1.08 (s, 3H), 1.04 (s, 3H), 0.96 (s, 3H), 0.95 (br. s., 3H).

4-((1R,3aR,5aR,5bR,7aR,11aS,11bR,13aR,13bS)-3a#2-(1,1-dioxidothiomorpholino)ethyl)amino)-5a,5b,8,8,11a-pentamethyl-1-(2,2,2-trifluoro-N-methylacetamido)-2,3,3a,4,5,5a,5b,6,7,7a,8,11,11a,11b,12,13,13a,13b-octadecahydro-1H-cyclopenta[a]chrysen-9-yl)benzoic acid was the second compound to elute from preparative HPLC. This product was isolated as a white powder TFA salt (0.0042 g, 18% yield). LCMS: m/z=776.5 (M+H)⁺, 2.13 min (method 5).

### Example B45

### Preparation of 4-((1R,3aR,5aR,5bR,7aR,11aS,11bR,13aR,13bS)-3a-((2-(1,1-dioxidothiomorpholino)ethyl)amino)-5a,5b,8,8,11a-pentamethyl-1-(4-(3-(trifluoromethyl)-3H-diazirin-3-yl)benzamido)-2,3,3a,4,5,5a,5b,6,7,7a,8,11,11a,11b,12,13,13a,13b-octadecahydro-1H-cyclopenta[a]chrysen-9-yl)benzoic acid.

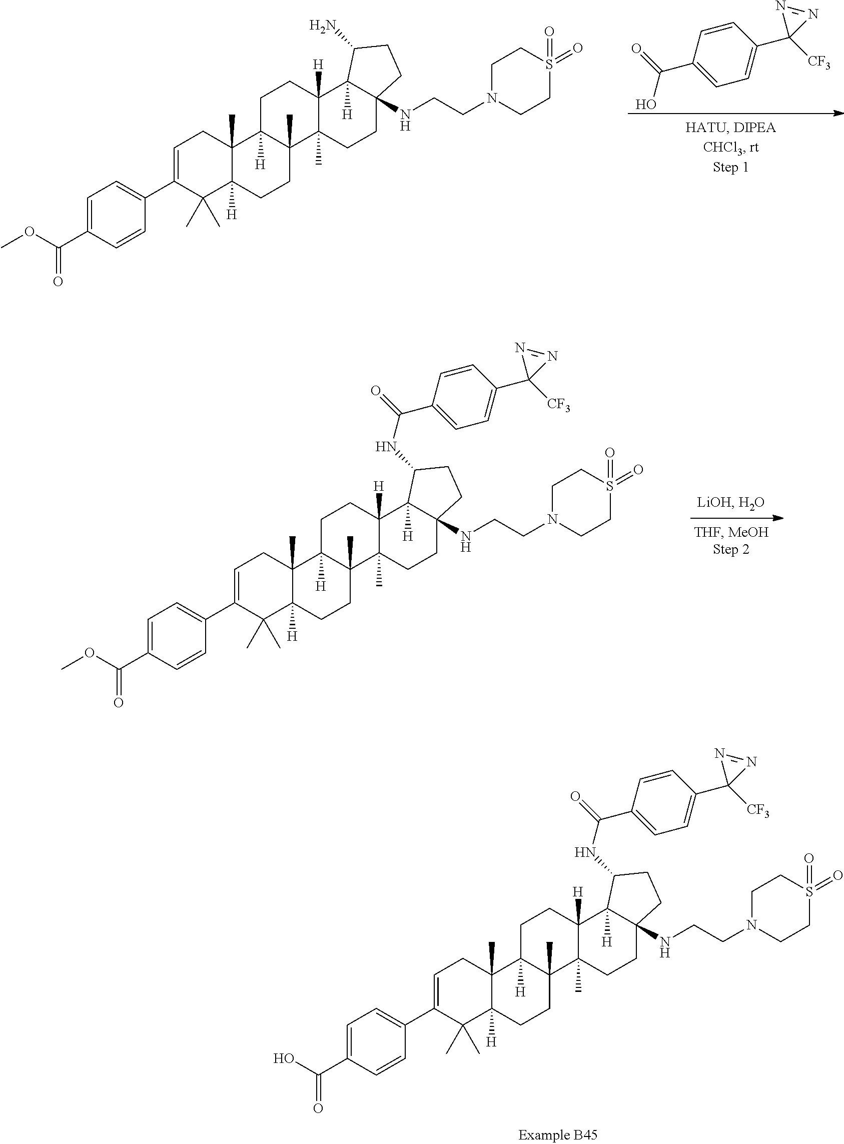

### Step 1. Preparation of methyl 4-((1R,3aR,5aR,5bR,7aR,11aS,11bR,13aR,13bS)-3a-((2-(1,1-dioxidothiomorpholino)ethyl)amino)-5a,5b,8,8,11a-pentamethyl-1-(4-(3-(trifluoromethyl)-3H-diazirin-3-yl)benzamido)-2,3,3a,4,5,5a,5b,6,7,7a,8,11,11a,11b,12,13,13a,13b-octadecahydro-1H-cyclopenta[a]chrysen-9-yl)benzoate.

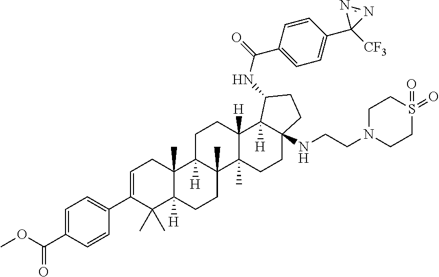

In a 1 dram vial were combined methyl 4-((1R,3aR,5aR,5bR,7aR,11aS,11bR,13aR,13bS)-1-amino-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (0.025 g, 0.037 mmol) and 4-(3-(trifluoromethyl)-3H-diazirin-3-yl)benzoic acid (11.00 mg, 0.048 mmol) with HATU (0.022 g, 0.059 mmol) and DIPEA (0.019 mL, 0.110 mmol) in chloroform (1 mL). The vial sealed with a PTFE lined screwcap, wrapped in aluminum foil, and the contents were stirred at rt overnight. The crude mixture was concentrated under a nitrogen stream to a residue, then the mixture was redissolved in a minimum quantity of methanol. The crude mixture was purified by reverse phase preparative HPLC (Prep HPLC Method 16). The desired product was isolated as a white solid TFA salt (0.0290 g, 80% yield). LCMS: m/z=892.6 (M+H)⁺, 2.41 min (method 5).

Step 2: In a 1 dram vial, methyl 4-((1R,3aR,5aR,5bR,7aR,11aS,11bR,13aR,13bS)-3a-((2-(1,1-dioxidothiomorpholino)ethyl)amino)-5a,5b,8,8,11a-pentamethyl-1-(4-(3-(trifluoromethyl)-3H-diazirin-3-yl)benzamido)-2,3,3a,4,5,5a,5b,6,7,7a,8,11,11a,11b,12,13,13a,13b-octadecahydro-1H-cyclopenta[a]chrysen-9-yl)benzoate (0.0295 g, 0.033 mmol) was dissolved in a mixture of tetrahydrofuran (0.3 mL) and MeOH (0.3 mL) and the mixture was then treated with 1M aqueous lithium hydroxide hydrate (0.132 mL, 0.132 mmol). The vial was wrapped in aluminum foil to block light from entering, sealed with a PTFE lined screwcap and the mixture was heated to 70° C. with stirring for 45 min. The crude mixture was purified by reverse phase preparative HPLC (Prep HPLC Method 19) to provide the desired product as a white solid TFA salt (0.0190 g, 53.9% yield). LCMS: m/z=878.6 (M+H)⁺, 2.25 min (method 5). ¹H NMR (400 MHz, 1:1 mixture of chloroform-d and methanol-d₄, methanol-d₄ lock) δ 8.63 (d, J=7.8 Hz, 1H), 7.92 (d, J=8.3 Hz, 2H), 7.85 (d, J=8.6 Hz, 2H), 7.28 (d, J=8.1 Hz, 2H), 7.19 (d, J=8.3 Hz, 2H), 5.27 (d, J=4.6 Hz, 1H), 4.50 (br. s., 1H), 3.30-2.87 (m, 12H), 2.53-2.33 (m, 2H), 2.20-1.75 (m, 8H), 1.75-1.64 (m, 2H), 1.64-1.35 (m, 9H), 1.26 (br. s., 3H), 1.19 (s, 3H), 1.10 (s, 3H), 1.02 (s, 3H), 0.95 (br. s., 3H), 0.94 (br. s., 3H).

### Example B46

### Preparation of 4-((1R,3aS,5aR,5bR,7aR,11aS,11bR,13aR,13bS)-1-acetyl-3a-amino-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid.

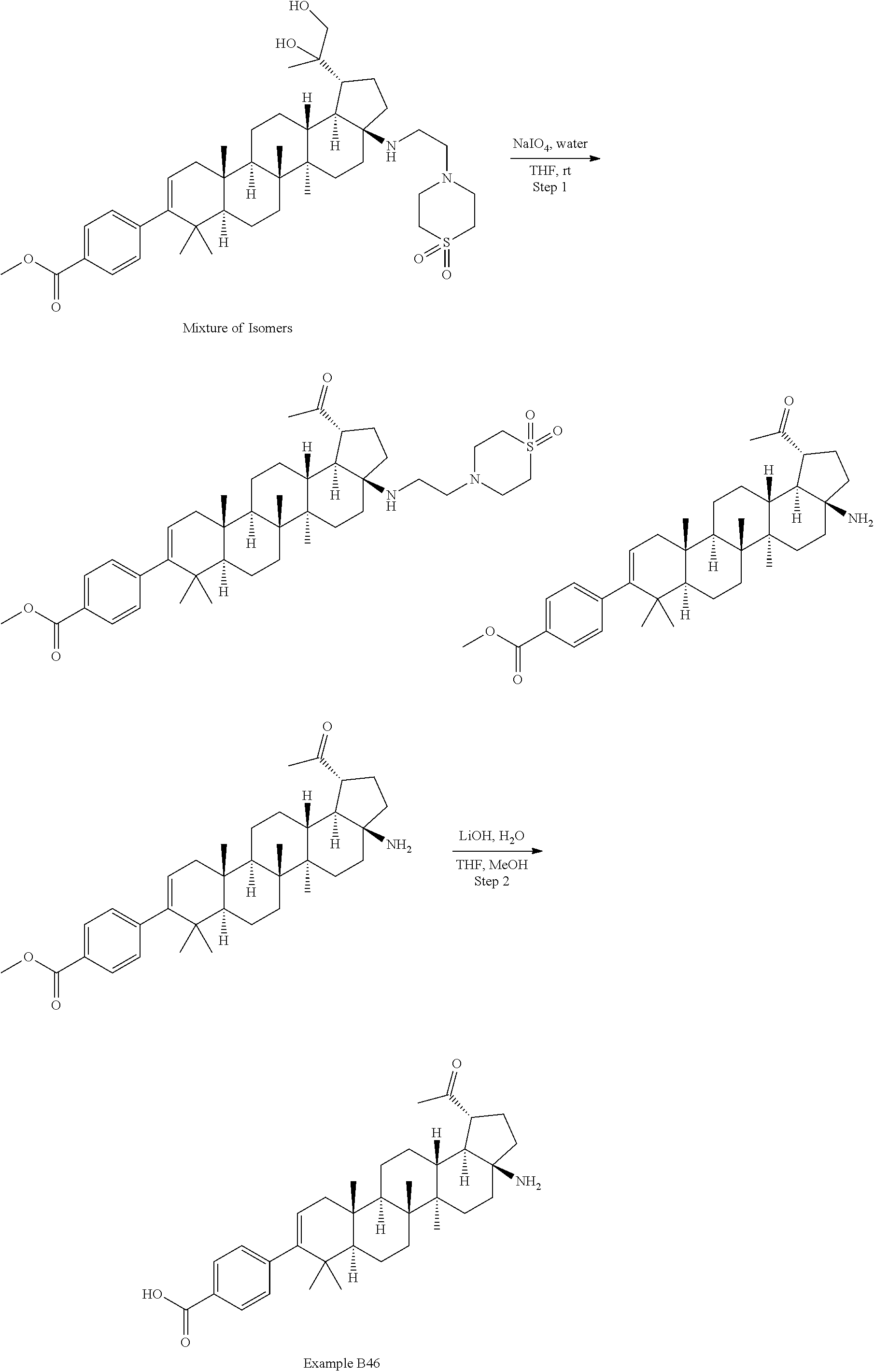

### Step 1. Preparation of methyl 4-((1R,3aS,5aR,5bR,7aR,11aS,11bR,13aR,13bS)-1-acetyl-3a-amino-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate.

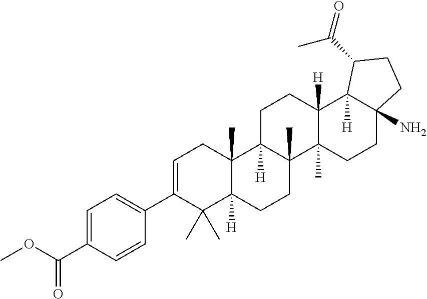

A mixture diol diastereomers methyl 4-((1R,3aS,5aR,5bR,7aR,11aS,11bR,13aR,13bS)-1-(1,2-dihydroxypropan-2-yl)-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (12.26 g, 16.59 mmol) was dissolved in a mixture of THF (450 mL) and water (150 mL) and the resulting solution was chilled in an ice bath. Sodium periodate (7.10 g, 33.2 mmol) was added. A clear solution quickly became cloudy and a white flocculent solid precipitated. The mixture was stirred for 30 min at rt and was then diluted with chloroform (1200 mL) and water (500 mL) and the resulting mixture was shaken and phases were separated. The aqueous was extracted again with chloroform (2×400 mL). The organics were combined, dried over sodium sulfate, filtered and concentrated in vacuo. Purification of the residue by silica gel column chromatography (300 g silica, elution gradient 100% DCM to 20:1 DCM:MeOH over 6 column volumes, hold 20:1 DCM:MeOH for 8 column volumes) provided the desired product as a white solid (1.70 g, 18.8% yield). LCMS: m/z=546.4 (M+H)⁺, 2.36 min (method 5). ¹H NMR (400 MHz, 1:1 mixture of chloroform-d and methanol-d₄, methanol-d₄ lock) δ 7.91 (d, J=8.3 Hz, 2H), 7.21 (d, J=8.6 Hz, 2H), 5.29 (dd, J=6.2, 1.6 Hz, 1H), 3.91 (s, 3H), 2.90 (td, J=11.3, 5.7 Hz, 1H), 2.43-2.19 (m, 5H), 2.18-2.06 (m, 1H), 2.00-1.91 (m, 2H), 1.85-1.44 (m, 12H), 1.44-1.34 (m, 4H), 1.27 (d, J=10.0 Hz, 1H), 1.14 (s, 3H), 1.13-1.08 (m, 1H), 1.07 (s, 3H), 1.02 (s, 3H), 0.95 (s, 3H), 0.94 (s, 3H).

Step 2: In a 1 dram vial were combined methyl 4-((1R,3aS,5aR,5bR,7aR,11aS,11bR,13aR,13bS)-1-acetyl-3a-amino-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (0.035 g, 0.064 mmol) with a mixture of tetrahydrofuran (0.3 mL) and MeOH (0.3 mL) and the mixture was then treated with 1M aqueous lithium hydroxide hydrate (0.257 mL, 0.257 mmol). The vial was sealed with a PTFE lined screwcap and the mixture was heated to 70° C. with stirring for 45 min. The crude mixture was purified by reverse phase preparative HPLC (Prep HPLC Method 16) to afford 4-((1R,3aS,5aR,5bR,7aR,11aS,11bR,13aR,13bS)-1-acetyl-3a-amino-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid as a white powder TFA salt (0.0253 g, 60% yield). LCMS: m/z=532 (M+H)⁺, 2.11 min (method 5). ¹H NMR (400 MHz, 1:1 mixture of chloroform-d and methanol-d₄, methanol-d₄ lock) δ 7.92 (d, J=8.6 Hz, 2H), 7.20 (d, J=8.6 Hz, 2H), 5.29 (dd, J=6.1, 1.5Hz, 1H), 2.88 (td, J=11.2, 6.1 Hz, 1H), 2.36 (t, J=11.7 Hz, 1H), 2.31-2.20 (m, 4H), 2.13 (dd, J=17.1, 6.4 Hz, 1H), 2.00-1.89 (m, 2H), 1.87-1.47 (m, 11H), 1.47-1.33 (m, 3H), 1.31-1.23 (m, 1H), 1.14 (s, 3H), 1.10 (br. s., 1H), 1.07 (s, 3H), 1.03 (s, 3H), 0.95 (s, 3H), 0.94 (s, 3H).

### Example B47

### Preparation of 4-((1R,3aS,5aR,5bR,7aR,11aS,11bR,13aR,13bS)-1-acetyl-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoic acid.

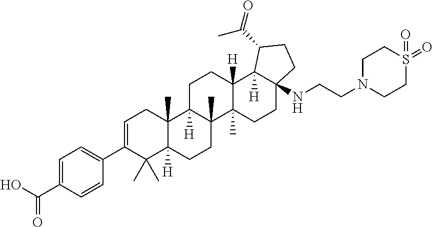

In a 1 dram vial were combined methyl 4-((1R,3aS,5aR,5bR,7aR,11aS,11bR,13aR,13bS)-1-acetyl-3a-((2-(1,1-dioxidothiomorpholino)ethyl)amino)-5a,5b,8,8,11a-pentamethyl-2,3,3a,4,5,5a,5b,6,7,7a,8,11,11a,11b,12,13,13a,13b-octadecahydro-1H-cyclopenta[a]chrysen-9-yl)benzoate (0.0262 g, 0.028 mmol) with lithium hydroxide monohydrate, 1.0M aqueous solution (0.249 mL, 0.249 mmol), tetrahydrofuran (0.3 mL) and MeOH (0.3 mL). The vial was sealed with a PTFE lined screwcap and the mixture was heated to 70° C. with stirring for 35 min. The crude mixture was purified by reverse phase preparative HPLC (Prep HPLC Method 2) to afford the title compound as a white powder TFA salt (0.0208 g, 80% yield). LCMS: m/z=693.6 (M+H)⁺, 2.16 min (method 5). ¹H NMR (400 MHz, 1:1 mixture of chloroform-d and methanol-d₄, methanol-d₄ lock) δ 7.92 (d, J=8.3 Hz, 2H), 7.20 (d, J=8.3 Hz, 2H), 5.33-5.26 (m, 1H), 3.31-2.98 (m, 12H), 2.57 (t, J=11.6 Hz, 1H), 2.44-2.33 (m, 1H), 2.26 (s, 3H), 2.19-2.01 (m, 3H), 1.88-1.75 (m, 2H), 1.75-1.64 (m, 3H), 1.64-1.50 (m, 5H), 1.50-1.38 (m, 4H), 1.32-1.23 (m, 2H), 1.19 (s, 3H), 1.16-1.06 (m, 5H), 1.03 (s, 3H), 0.96 (s, 3H), 0.95 (s, 3H).

### Example B48

### Preparation of (1R,3aS,5aR,5bR,7aR,11aS,11bR,13aR,13bR)-9-(4-carboxyphenyl)-5a,5b,8,8,11a-pentamethyl-1-(1-methylcyclopropyl)-2,3,3a,4,5,5a,5b,6,7,7a,8,11,11a,11b,12,13,13a,13b-octadecahydro-1H-cyclopenta[a]chrysene-3a-carboxylic acid.

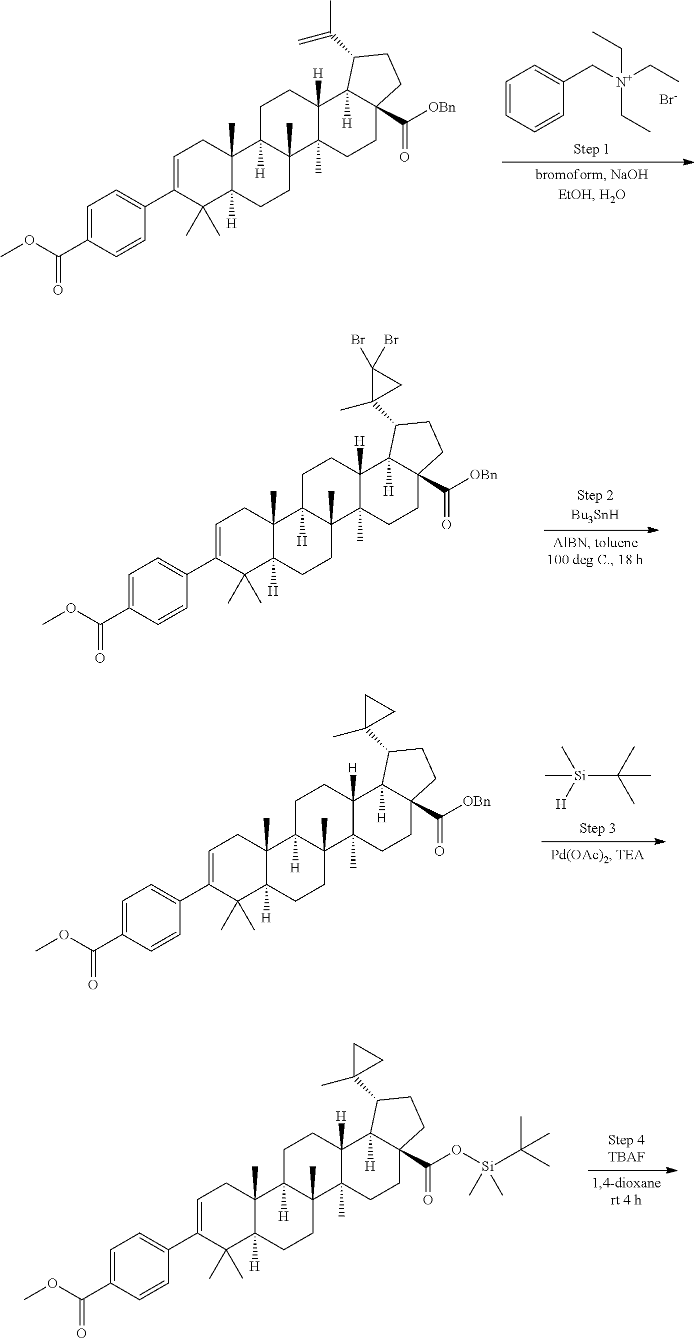

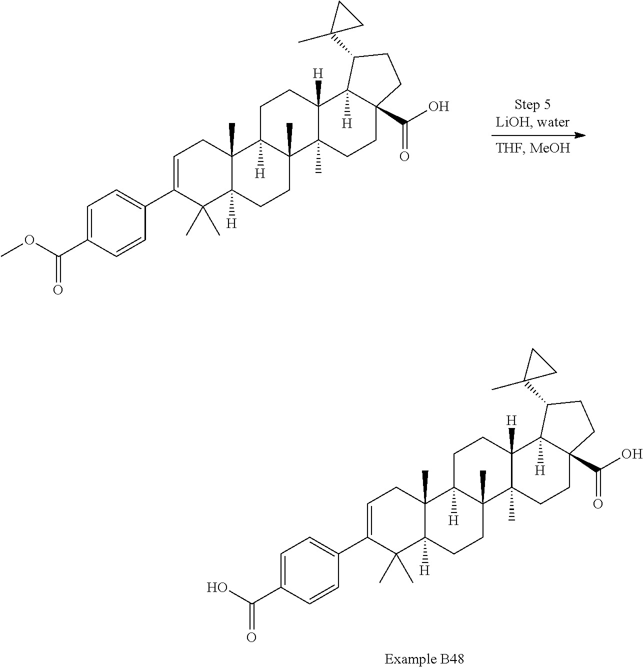

### Step 1. Preparation of (1R,3aS,5aR,5bR,7aR,11aS,11bR,13aR,13bS)-benzyl 1-((S)-2,2-dibromo-1-methylcyclopropyl)-9-(4-(methoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylate.

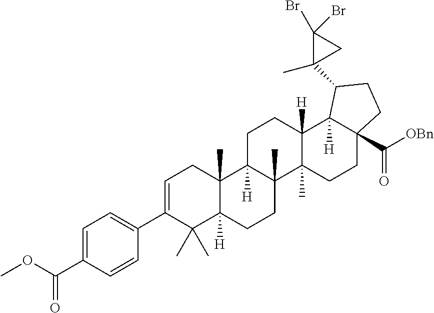

A solution of (1R,3aS,5aR,5bR,7aR,11aS,11bR,13aR,13bR)-benzyl 9-(4-(methoxycarbonyl)phenyl)-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysene-3a-carboxylate (11.0 g, 16.6 mmol) and N-benzyl-N,N-diethylethanaminium bromide (0.903 g, 3.32 mmol) in bromoform (29.0 mL, 332 mmol) was stirred rapidly and treated slowly with an aqueous (50 mL) solution of sodium hydroxide (13.3 g, 332 mmol). The resulting biphasic mixture was heated to 63 degrees C. with rapid stirring. Additional bromoform (29.0 mL, 332 mmol)) and aqueous (50 mL) NaOH (13.3 g, 332 mmol) were added, and the mixture was stirred rapidly at 63 degrees C. for 60 h. The mixture was slowly diluted with water (700 mL) and washed with DCM (3×200 mL). The organic extracts were combined and concentrated in vacuo to a light brown oil Purification by silica gel column chromatography (gradient 100% hexanes to 30:1 hexanes:EtOAc) gave 6.85 g (49.5% yield) of a white solid. ¹H NMR (500 MHz, CHLOROFORM-d) δ ppm 7.95-7.89 (m, J=8.2Hz, 2H), 7.40-7.28 (m, 5H), 7.22-7.16 (m, J=8.2Hz, 2H), 5.29 (dd, J=6.1, 1.5Hz, 1H), 5.17 (d, J=12.2Hz, 1H), 5.03 (d, J=12.2Hz, 1H), 3.90 (s, 3H), 2.35-2.23 (m, 3H), 2.20-2.06 (m, 2H), 1.98 (dd, J=12.5, 7.6 Hz, 1H), 1.71-1.55 (m, 5H), 1.55 (s, 3H), 1.51-1.27 (m, 12H), 1.26 (s, 5H), 1.20 (dd, J=10.8, 2.9 Hz, 1H), 1.18-1.07 (m, 2H), 0.98 (s, 3H), 0.95 (s, 3H), 0.91 (d, J=2.4 Hz, 6H), 0.88 (t, J=6.9 Hz, 2H), 0.81 (s, 3H).

### Step 2. Preparation of (1R,3aS,5aR,5bR,7aR,11aS,11bR,13aR,13bR)-benzyl 9-(4-(methoxycarbonyl)phenyl)-5a,5b,8,8,11a-pentamethyl-1-(1-methylcyclopropyl)-2,3,3a,4,5,5a,5b,6,7,7a,8,11,11a,11b,12,13,13a,13b-octadecahydro-1H-cyclopenta[a]chrysene-3a-carboxylate.

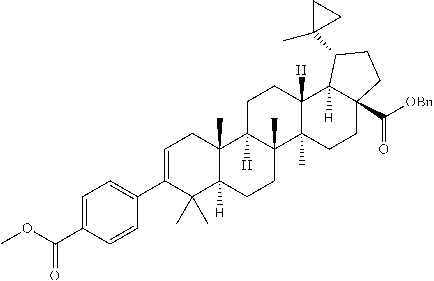

A solution of (1R,3aS,5aR,5bR,7aR,11aS,11bR,13aR,13bS)-benzyl 1-((S)-2,2-dibromo-1-methylcyclopropyl)-9-(4-(methoxycarbonyl)phenyl)-5a,5b,8,8,11a-pentamethyl-2,3,3a,4,5,5a,5b,6,7,7a,8,11,11a,11b,12,13,13a,13b-octadecahydro-1H-cyclopenta[a]chrysene-3a-carboxylate (6.82 g, 8.17 mmol) in toluene (100 mL) was treated with tri-n-butyltin hydride (14.3 g, 13.1 mL, 49.0 mmol) followed by AIBN (0.067 g, 0.409 mmol). The mixture was heated at 100° C. with stirring for 18 h. The mixture was concentrated in vacuo and purified by silica gel column chromatography (gradient 100% hexanes to 25:1 hexanes:EtOAc). Product fractions were combined and concentrated in vacuo, then the residue was redissolved in DCM and passed through a column comprised of 90% silica and 10% KF by weight using DCM as the eluent. Concentration in vacuo provided 5.48 g (99%) of a white foam solid. ¹H NMR (500 MHz, CHLOROFORM-d) δ ppm 8.00-7.91 (m, J=8.2Hz, 2H), 7.42-7.31 (m, 5H), 7.26-7.18 (m, J=8.2Hz, 2H), 5.36-5.29 (m, 1H), 5.15 (d, J=12.2Hz, 1H), 5.08 (d, J=12.5Hz, 1H), 3.94 (s, 3H), 2.32-2.24 (m, 1H), 2.24-2.11 (m, 2H), 2.07-1.97 (m, 1H), 1.94-1.87 (m, 1H), 1.87-1.78 (m, 1H), 1.77-1.67 (m, 2H), 1.67-1.59 (m, 1H), 1.56-1.36 (m, 10H), 1.36-1.26 (m, 2H), 1.26-1.20 (m, 2H), 1.19-1.13 (m, 1H), 1.02 (s, 3H), 1.00 (s, 3H), 0.95 (br. s., 3H), 0.95 (br. s., 3H), 0.94 (br. s., 3H), 0.85 (s, 3H), 0.46-0.40 (m, 1H), 0.38 (dt, J=9.2, 4.7 Hz, 1H), 0.31-0.20 (m, 2H).

### Step 3. Preparation of (1R,3aS,5aR,5bR,7aR,11aS,11bR,13aR,13bR)-tert-butyldimethylsilyl 9-(4-(methoxycarbonyl)phenyl)-5a,5b,8,8,11a-pentamethyl-1-(1-methylcyclopropyl)-2,3,3a,4,5,5a,5b,6,7,7a,8,11,11a,11b,12,13,13a,13b-octadecahydro-1H-cyclopenta[a]chrysene-3a-carboxylate.

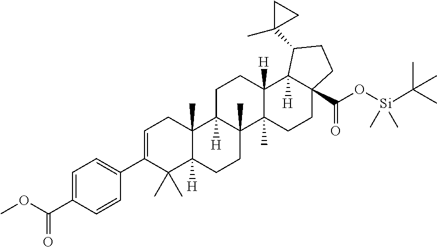

To a solution of (1R,3aS,5aR,5bR,7aR,11aS,11bR,13aR,13bR)-benzyl 9-(4-(methoxycarbonyl)phenyl)-5a,5b,8,8,11a-pentamethyl-1-(1-methylcyclopropyl)-2,3,3a,4,5,5a,5b,6,7,7a,8,11,11a,11b,12,13,13a,13b-octadecahydro-1H-cyclopenta[a]chrysene-3a-carboxylate (5.34 g, 7.89 mmol) in 1,2-dichloroethane (100 mL) were added triethylamine (1.76 mL, 12.6 mmol), tert-butyldimethylsilane (2.62 mL, 1.84 g, 15.8 mmol) and palladium (II) acetate (0.443 g, 1.97 mmol). The mixture was heated to 60° C. for 22 h. The crude reaction mixture was passed through a silica gel/celite plug with 10:1 hexanes:EtOAc as the eluent. Concentration in vacuo gave 6.34 g (>100% yield) of a white solid which was carried to the next step without further purification.

### Step 4. Preparation of (1R,3aS,5aR,5bR,7aR,11aS,11bR,13aR,13bR)-9-(4-(methoxycarbonyl)phenyl)-5a,5b,8,8,11a-pentamethyl-1-(1-methylcyclopropyl)-2,3,3a,4,5,5a,5b,6,7,7a,8,11,11a,11b,12,13,13a,13b-octadecahydro-1H-cyclopenta[a]chrysene-3a-carboxylic acid.

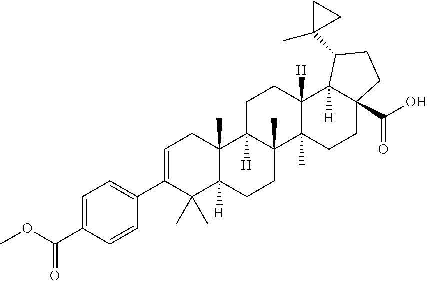

To a solution of (1R,3aS,5aR,5bR,7aR,11aS,11bR,13aR,13bR)-tert-butyldimethylsilyl 9-(4-(methoxycarbonyl)phenyl)-5a,5b,8,8,11a-pentamethyl-1-(1-methylcyclopropyl)-2,3,3a,4,5,5a,5b,6,7,7a,8,11,11a,11b,12,13,13a,13b-octadecahydro-1H-cyclopenta[a]chrysene-3a-carboxylate (5.53 g, 7.89 mmol) in 1,4-dioxane (100 mL) was added TBAF, 1.0M in THF (11.8 mL, 11.8 mmol). The mixture was stirred for 2.5 h, then 400 mL of 1M aqueous HCl was added and the resulting suspension of white solid was stirred for 10 min at rt. The solid precipitate was isolated by filtration and dried to afford the title compound as a white powder (4.43 g, 96% yield). LCMS: m/e 587.4 (M+H)⁺, 4.58 min (method 5). ¹H NMR (500 MHz, CHLOROFORM-d) δ ppm 9.61 (br. s., 1H), 8.01-7.90 (m, 2H), 7.26-7.18 (m, 2H), 5.33 (dd, J=6.3, 1.7 Hz, 1H), 3.94 (s, 3H), 2.34-2.23 (m, 1H), 2.23-2.13 (m, 2H), 2.08-1.99 (m, 1H), 1.99-1.90 (m, 2H), 1.80-1.62 (m, 3H), 1.60-1.36 (m, 12H), 1.34-1.19 (m, 4H), 1.05 (s, 3H), 1.04-0.99 (m, 6H), 0.98-0.93 (m, 9H), 0.47-0.41 (m, 1H), 0.41-0.36 (m, 1H), 0.33-0.22 (m, 2H).

Step 5: In a 20 mL scintillation vial were combined (1R,3aS,5aR,5bR,7aR,11aS,11bR,13aR,13bR)-9-(4-(methoxycarbonyl)phenyl)-5a,5b,8,8,11a-pentamethyl-1-(1-methylcyclopropyl)-2,3,3a,4,5,5a,5b,6,7,7a,8,11,11a,11b,12,13,13a,13b-octadecahydro-1H-cyclopenta[a]chrysene-3a-carboxylic acid (0.080 g, 0.136 mmol) with lithium hydroxide monohydrate (0.023 g, 0.545 mmol) in THF (1 mL), methanol (1 mL) and water (0.5 mL). The suspended mixture was warmed to 60° C. for 1 h. The crude mixture was purified by reverse phase preparative HPLC (Prep HPLC method 26). The desired product was obtained as a white powder (0.042 g, 51.6% yield). LCMS: m/e 571.7 (M−H)⁻, 2.35 min (method 3). ¹H NMR (500 MHz, 1:1 mixture of CDCl₃ and MeOD, MeOD lock) d 7.88 (d, J=8.2Hz, 2H), 7.16 (d, J=8.2Hz, 2H), 5.29-5.21 (m, 1H), 2.21-2.13 (m, 2H), 2.10 (dd, J=17.1, 6.4 Hz, 1H), 1.99-1.91 (m, 1H), 1.88-1.78 (m, 2H), 1.67 (d, J=17.1 Hz, 1H), 1.65-1.53 (m, 2H), 1.51-1.38 (m, 8H), 1.38-1.26 (m, 4H), 1.25-1.12 (m, 4H), 0.98 (s, 3H), 0.95 (s, 3H), 0.94 (br. s., 3H), 0.88 (s, 4H), 0.88 (br. s., 3H), 0.38-0.27 (m, 2H), 0.23-0.12 (m, 2H).

### Example B49

### Preparation of 4-((1R,3aS,5aR,5bR,7aR,11aS,11bR,13aR,13bR)-3a-amino-5a,5b,8,8,11a-pentamethyl-1-(1-methylcyclopropyl)-2,3,3a,4,5,5a,5b,6,7,7a,8,11,11a,11b,12,13,13a,13b-octadecahydro-1H-cyclopenta[a]chrysen-9-yl)benzoic acid.

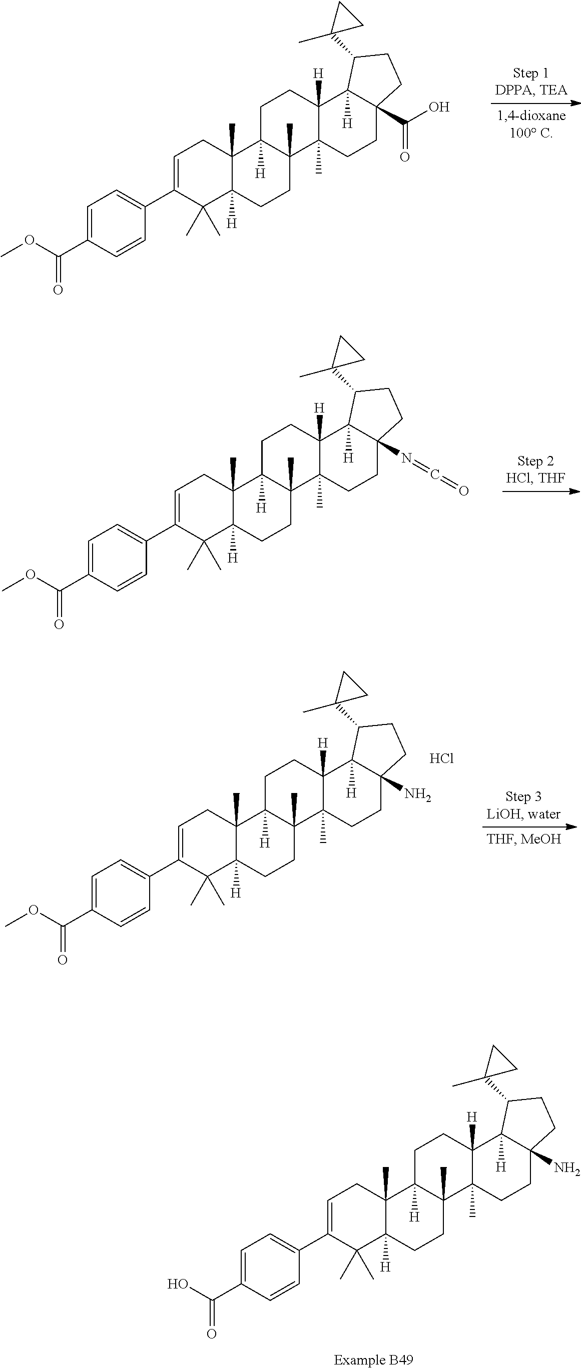

### Step 1. Preparation of methyl 4-((1R,3aS,5aR,5bR,7aR,11aS,11bR,13aR,13bR)-3a-isocyanato-5a,5b,8,8,11a-pentamethyl-1-(1-methylcyclopropyl)-2,3,3a,4,5,5a,5b,6,7,7a,8,11,11a,11b,12,13,13a,13b-octadecahydro-1H-cyclopenta[a]chrysen-9-yl)benzoate.

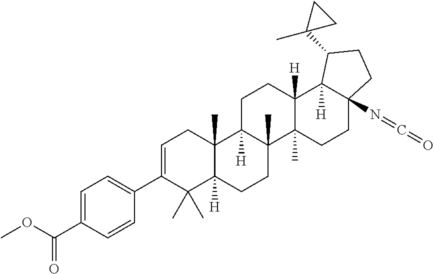

To a slurry of (1R,3aS,5aR,5bR,7aR,11aS,11bR,13aR,13bR)-9-(4-(methoxycarbonyl)phenyl)-5a,5b,8,8,11a-pentamethyl-1-(1-methylcyclopropyl)-2,3,3a,4,5,5a,5b,6,7,7a,8,11,11a,11b,12,13,13a,13b-octadecahydro-1H-cyclopenta[a]chrysene-3a-carboxylic acid (3.50 g, 5.96 mmol) in 1,4-dioxane (60 mL) was added triethylamine (1.50 mL, 10.7 mmol) and diphenylphosphoryl azide (1.93 mL, 2.46 g, 8.95 mmol). The resulting slurry was heated to 100° C. for 5 h. The mixture was cooled to rt, diluted with EtOAc and washed with 1N NaOH (2×70 mL) and then with brine (25 mL). Solids crashed out of the organic phase and were isolated by filtration. Concentration of the filtrate afforded another crop of precipitate. The two initial precipitates were both the same material and dried to a white powder (2.25 g, 64.6% yield). ¹H NMR (400 MHz, CHLOROFORM-d) δ ppm 8.01-7.92 (m, J=8.3 Hz, 2H), 7.26-7.18 (m, J=8.3 Hz, 2H), 5.34 (dd, J=6.1, 1.7 Hz, 1H), 3.93 (s, 3H), 2.18 (dd, J=17.1, 6.4 Hz, 1H), 2.13-1.99 (m, 2H), 1.92-1.64 (m, 6H), 1.63-1.36 (m, 11H), 1.32-1.18 (m, 4H), 1.13 (s, 3H), 1.04 (s, 3H), 1.01 (s, 3H), 0.97 (s, 3H), 0.96 (s, 3H), 0.95 (s, 3H), 0.46-0.38 (m, 2H), 0.38-0.25 (m, 2H).

### Step 2. Preparation of methyl 4-((1R,3aS,5aR,5bR,7aR,11aS,11bR,13aR,13bR)-3a-amino-5a,5b,8,8,11a-pentamethyl-1-(1-methylcyclopropyl)-2,3,3a,4,5,5a,5b,6,7,7a,8,11,11a,11b,12,13,13a,13b-octadecahydro-1H-cyclopenta[a]chrysen-9-yl)benzoate hydrochloride.

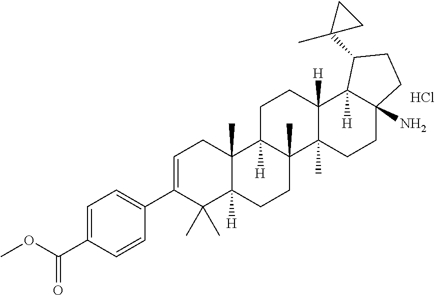

A solution of methyl 4-((1R,3aS,5aR,5bR,7aR,11aS,11bR,13aR,13bR)-3a-isocyanato-5a,5b,8,8,11a-pentamethyl-1-(1-methylcyclopropyl)-2,3,3a,4,5,5a,5b,6,7,7a,8,11,11a,11b,12,13,13a,13b-octadecahydro-1H-cyclopenta[a]chrysen-9-yl)benzoate (2.24 g, 3.84 mmol) in THF (40 mL) was treated with concentrated HCl (7.93 mL, 96 mmol). The resulting solution was stirred at rt for 48 h. The mixture was filtered to remove solids and the filtrate was then concentrated in vacuo to a white powder (2.30 g, 100% yield). LCMS: m/e 559 (M+H)⁺, 2.12 min (method 6). ¹H NMR (400 MHz, CHLOROFORM-d) δ ppm 8.07 (br. s., 2H), 7.98-7.91 (m, J=8.1 Hz, 2H), 7.26-7.17 (m, J=8.1 Hz, 2H), 5.34 (d, J=5.1 Hz, 1H), 3.93 (s, 3H), 2.43 (dd, J=12.7, 9.0 Hz, 1H), 2.31-2.10 (m, 4H), 2.06-1.83 (m, 4H), 1.78-1.67 (m, 4H), 1.65-1.44 (m, 11H), 1.30 (br. s., 6H), 1.06 (s, 3H), 1.04 (s, 3H), 0.98 (br. s., 3H), 0.96 (br. s., 3H), 0.96 (br. s., 3H), 0.54 (br. s., 1H), 0.42 (d, J=5.1 Hz, 2H), 0.35-0.23 (m, 1H).

Step 3: A mixture of methyl 4-((1R,3aS,5aR,5bR,7aR,11aS,11bR,13aR,13bR)-3a-amino-5a,5b,8,8,11a-pentamethyl-1-(1-methylcyclopropyl)-2,3,3a,4,5,5a,5b,6,7,7a,8,11,11a,11b,12,13,13a,13b-octadecahydro-1H-cyclopenta[a]chrysen-9-yl)benzoate hydrochloride (0.050 g, 0.090 mmol) and lithium hydroxide monohydrate, 1.0M aqueous (0.359 mL, 0.359 mmol) in THF (0.8 mL) and MeOH (0.8 mL) in a sealed vial was warmed to 75° C. to provide a homogeneous solution. The mixture was removed from heat after 1 h. Purification of the crude mixture by reverse phase preparative HPLC using Prep HPLC Method 24 gave 4-((1R,3aS,5aR,5bR,7aR,11aS,11bR,13aR,13bR)-3a-amino-5a,5b,8,8,11a-pentamethyl-1-(1-methylcyclopropyl)-2,3,3a,4,5,5a,5b,6,7,7a,8,11,11a,11b,12,13,13a,13b-octadecahydro-1H-cyclopenta[a]chrysen-9-yl)benzoic acid as a slightly yellow powder (48.5 mg, 79% yield). LCMS: m/e 544.7 (M+H)⁺, 1.94 min (method 6). ¹H NMR (400 MHz, 1:1 mixture of CDCl₃ and MeOD, MeOD lock) δ ppm 6.68 (d, J=8.3 Hz, 2H), 5.96 (d, J=8.0 Hz, 2H), 4.14-4.00 (m, 1H), 2.09 (dt, J=3.2, 1.5Hz, 1H), 1.00-0.86 (m, 2H), 0.86-0.64 (m, 2H), 0.60-0.44 (m, 5H), 0.43-0.22 (m, 10H), 0.18-0.02 (m, 4H), −0.04-−0.14 (m, 4H), −0.17 (s, 3H), −0.20 (s, 3H), −0.27 (s, 4H), −0.28 (s, 3H), −0.29 (br. s., 3H), −0.73-−0.87 (m, 2H), −0.87-−0.97 (m, 2H).

### Example B50

### Preparation of 4-((1R,3aS,5aR,5bR,7aR,11aS,11bR,13aR,13bR)-5a,5b,8,8,11a-pentamethyl-3a-(methylamino)-1-(1-methylcyclopropyl)-2,3,3a,4,5,5a,5b,6,7,7a,8,11,11a,11b,12,13,13a,13b-octadecahydro-1H-cyclopenta[a]chrysen-9-yl)benzoic acid.

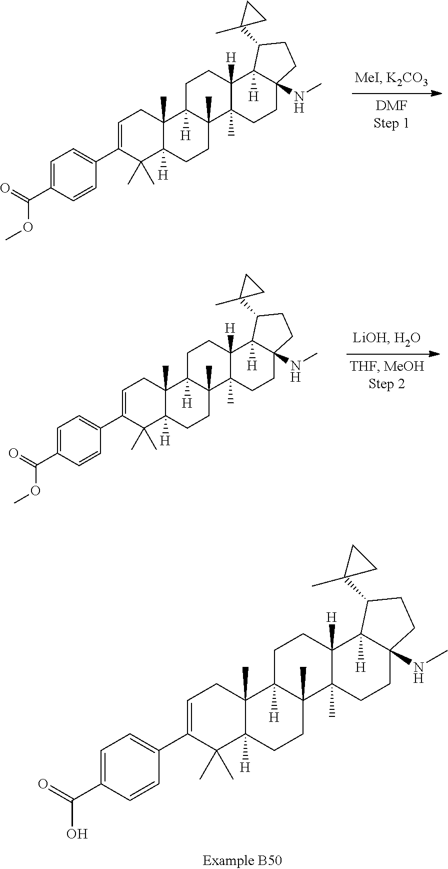

### Step 1. Preparation of methyl 4-((1R,3aS,5aR,5bR,7aR,11aS,11bR,13aR,13bR)-5a,5b,8,8,11a-pentamethyl-3a-(methylamino)-1-(1-methylcyclopropyl)-2,3,3a,4,5,5a,5b,6,7,7a,8,11,11a,11b,12,13,13a,13b-octadecahydro-1H-cyclopenta[a]chrysen-9-yl)benzoate.

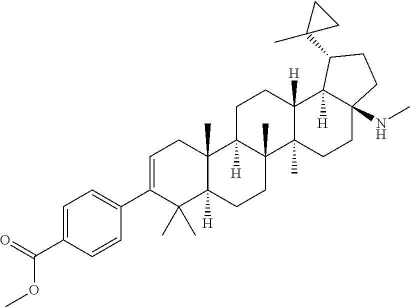

A mixture of methyl 4-((1R,3aS,5aR,5bR,7aR,11aS,11bR,13aR,13bR)-3a-amino-5a,5b,8,8,11a-pentamethyl-1-(1-methylcyclopropyl)-2,3,3a,4,5,5a,5b,6,7,7a,8,11,11a,11b,12,13,13a,13b-octadecahydro-1H-cyclopenta[a]chrysen-9-yl)benzoate hydrochloride (50 mg, 0.084 mmol) and potassium carbonate (23.3 mg, 0.168 mmol) in DMF (1 mL) in a sealed vial was treated with methyl iodide (0.016 mL, 0.036 g, 0.25 mmol) and heated to 75° C. for 3 h. The mixture was diluted with THF and brine. The mixture was shaken and the phases were separated. The organic was concentrated in vacuo to a residue. The product was isolated after reverse phase preparative HPLC purification using Prep HPLC Method 25 as a glassy white solid (57.2 mg, 99% yield) mono TFA salt. LCMS: m/e 572.6 (M+H)⁺, 2.18 min (method 6). ¹H NMR (400 MHz, CHLOROFORM-d) δ ppm 8.02-7.88 (m, J=8.1 Hz, 2H), 7.26-7.17 (m, J=8.1 Hz, 2H), 5.33 (d, J=4.6 Hz, 1H), 3.93 (s, 3H), 2.65 (br. s., 3H), 2.27-2.09 (m, 3H), 2.09-1.99 (m, 1H), 1.95 (dd, J=13.8, 8.4 Hz, 1H), 1.89-1.74 (m, 3H), 1.72-1.69 (m, 1H), 1.66 (d, J=2.9 Hz, 1H), 1.61-1.39 (m, 10H), 1.38-1.24 (m, 4H), 1.14 (s, 3H), 1.10 (s, 3H), 1.04 (s, 3H), 0.99-0.91 (m, 9H), 0.49-0.40 (m, 2H), 0.40-0.35 (m, 1H), 0.35-0.27 (m, 1H).

Step 2: A mixture of methyl 4-((1R,3aS,5aR,5bR,7aR,11aS,11bR,13aR,13bR)-5a,5b,8,8,11a-pentamethyl-3a-(methylamino)-1-(1-methylcyclopropyl)-2,3,3a,4,5,5a,5b,6,7,7a,8,11,11a,11b,12,13,13a,13b-octadecahydro-1H-cyclopenta[a]chrysen-9-yl)benzoate TFA salt (57 mg, 0.083 mmol), 1.0M aqueous LiOH (0.417 mL, 0.417 mmol), THF (0.8 mL) and methanol (0.8 mL) in a sealed vial was warmed to 75° C. for 1 h, then at 60° C. for 18 h. The title compound, 4-((1R,3aS,5aR,5bR,7aR,11aS,11bR,13aR,13bR)-5a,5b,8,8,11a-pentamethyl-3a-(methylamino)-1-(1-methylcyclopropyl)-2,3,3a,4,5,5a,5b,6,7,7a,8,11,11a,11b,12,13,13a,13b-octadecahydro-1H-cyclopenta[a]chrysen-9-yl)benzoic acid was isolated after reverse phase preparative HPLC purification using Prep HPLC Method 24 as a white solid (44.1 mg, 69% yield) mono TFA salt. LCMS: m/e 558.7 (M+H)⁺, 1.98 min (method 6). ¹H NMR (400 MHz, 1:1 mixture of CDCl₃ and MeOD, MeOD lock) δ ppm 6.69 (d, J=8.0 Hz, 2H), 5.97 (d, J=8.0 Hz, 2H), 4.07 (d, J=4.8 Hz, 1H), 2.09 (dt, J=3.2, 1.5Hz, 1H), 1.35 (s, 3H), 1.01-0.86 (m, 2H), 0.86-0.72 (m, 2H), 0.72-0.56 (m, 3H), 0.52 (d, J=16.3 Hz, 2H), 0.39-0.10 (m, 11H), 0.05 (dd, J=10.9, 6.7 Hz, 1H), −0.01-−0.07 (m, 1H), −0.08 (s, 3H), −0.15 (s, 3H), −0.19 (s, 3H), −0.26 (s, 3H), −0.28 (br. s., 3H), −0.29 (br. s., 3H), −0.72-−0.81 (m, 1H), −0.83 (dd, J=6.7, 3.9 Hz, 1H), −0.86-−0.96 (m, 2H).

### Example B51

### Preparation of 4-((1R,3aS,5aR,5bR,7aR,11aS,11bR,13aR,13bR)-3a-(2-(1,1-dioxidothiomorpholino)acetamido)-5a,5b,8,8,11a-pentamethyl-1-(1-methylcyclopropyl)-2,3,3a,4,5,5a,5b,6,7,7a,8,11,11a,11b,12,13,13a,13b-octadecahydro-1H-cyclopenta[a]chrysen-9-yl)benzoic acid.

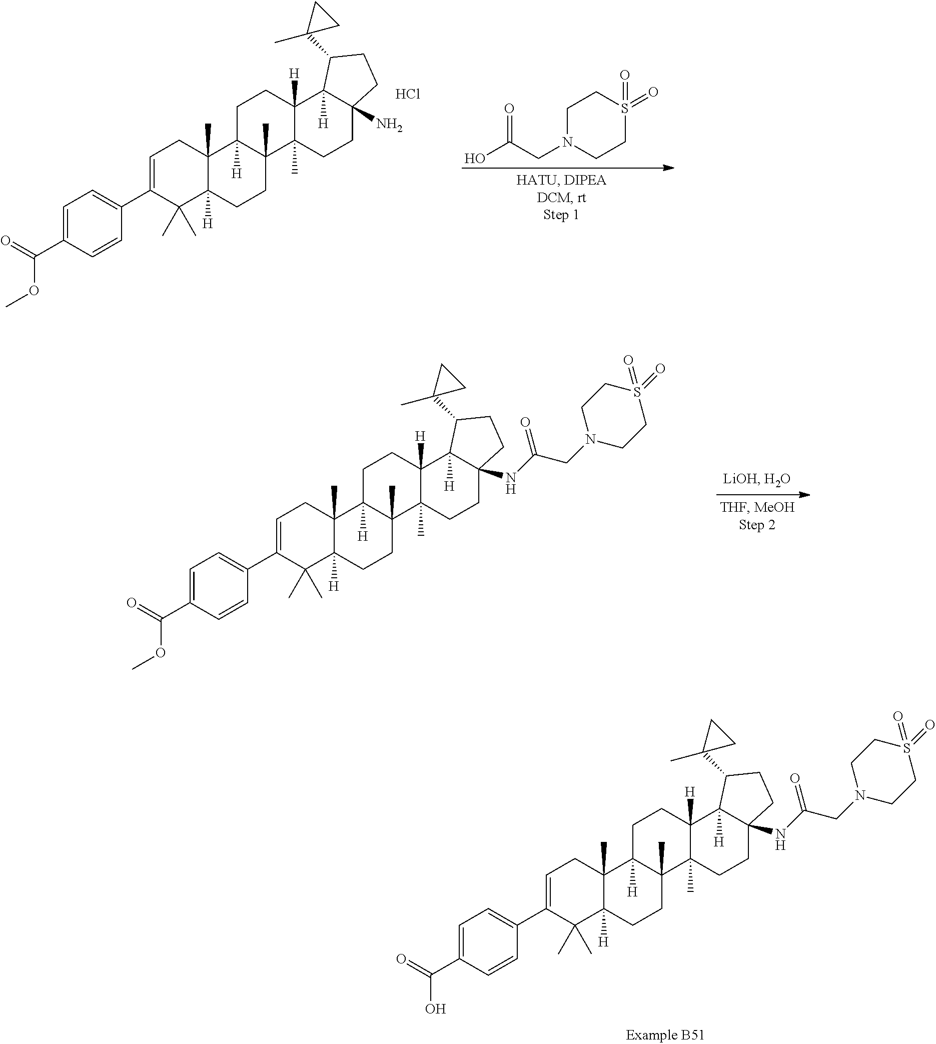

### Step 1. Preparation of methyl 4-((1R,3aS,5aR,5bR,7aR,11aS,11bR,13aR,13bR)-3a-(2-(1,1-dioxidothiomorpholino)acetamido)-5a,5b,8,8,11a-pentamethyl-1-(1-methylcyclopropyl)-2,3,3a,4,5,5a,5b,6,7,7a,8,11,11a,11b,12,13,13a,13b-octadecahydro-1H-cyclopenta[a]chrysen-9-yl)benzoate.

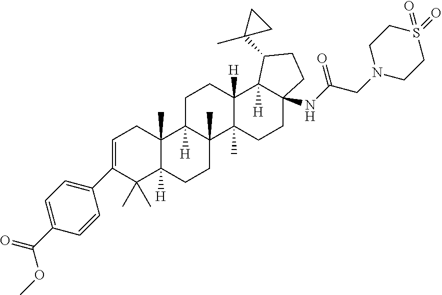

A mixture of methyl 4-((1R,3aS,5aR,5bR,7aR,11aS,11bR,13aR,13bR)-3a-amino-5a,5b,8,8,11a-pentamethyl-1-(1-methylcyclopropyl)-2,3,3a,4,5,5a,5b,6,7,7a,8,11,11a,11b,12,13,13a,13b-octadecahydro-1H-cyclopenta[a]chrysen-9-yl)benzoate hydrochloride (50 mg, 0.084 mmol) and 2-(1,1-dioxidothiomorpholino)acetic acid (0.023 g, 0.117 mmol) in DCM (1 mL) was treated with HATU (0.044 g, 0.117 mmol) and DIPEA (0.063 mL, 0.359 mmol). The mixture was stirred at rt for 3 h. The mixture was concentrated under nitrogen stream to a residue, then redissolved in a mixture of methanol and THF. The product was isolated after reverse phase preparative HPLC purification using Prep HPLC Method 25 as a glassy off-white solid (44.9 mg, 59% yield) mono TFA salt. LCMS: m/e 733.6 (M+H)⁺, 2.66 min (method 6). ¹H NMR (400 MHz, CHLOROFORM-d) δ ppm 8.01-7.90 (m, J=8.3 Hz, 2H), 7.26-7.18 (m, J=8.3 Hz, 2H), 5.34 (dd, J=6.1, 1.7 Hz, 1H), 3.94 (s, 3H), 3.35-3.20 (m, 6H), 3.12 (d, J=5.1 Hz, 4H), 2.71-2.56 (m, 1H), 2.46 (dd, J=12.5, 8.3 Hz, 1H), 2.19 (dd, J=17.0, 6.5Hz, 1H), 2.13-2.01 (m, 1H), 1.87-1.69 (m, 3H), 1.64-1.62 (m, 1H), 1.61-1.34 (m, 12H), 1.32-1.26 (m, 2H), 1.23-1.13 (m, 1H), 1.06 (s, 3H), 1.04 (s, 3H), 1.03 (br. s., 3H), 0.98 (s, 3H), 0.96 (s, 6H), 0.54-0.44 (m, 1H), 0.41-0.32 (m, 2H), 0.32-0.23 (m, 1H).

Step 2: A mixture of methyl 4-((1R,3aS,5aR,5bR,7aR,11aS,11bR,13aR,13bR)-3a-(2-(1,1-dioxidothiomorpholino)acetamido)-5a,5b,8,8,11a-pentamethyl-1-(1-methylcyclopropyl)-2,3,3a,4,5,5a,5b,6,7,7a,8,11,11a,11b,12,13,13a,13b-octadecahydro-1H-cyclopenta[a]chrysen-9-yl)benzoate trifluoroacetic acid salt (0.0449 g, 0.053 mmol) and 1M aqueous lithium hydroxide (0.265 mL, 0.265 mmol) with THF (0.5 mL) and MeOH (0.5 mL) in a sealed vial was heated to 75° C. for 1 h. The title compound, 4-((1R,3aS,5aR,5bR,7aR,11aS,11bR,13aR,13bR)-3a-(2-(1,1-dioxidothiomorpholino)acetamido)-5a,5b,8,8,11a-pentamethyl-1-(1-methylcyclopropyl)-2,3,3a,4,5,5a,5b,6,7,7a,8,11,11a,11b,12,13,13a,13b-octadecahydro-1H-cyclopenta[a]chrysen-9-yl)benzoic acid was isolated after reverse phase preparative HPLC purification using Prep HPLC Method 24 as a white solid (37.6 mg, 83% yield) mono TFA salt. LCMS: m/e 719.7 (M+H)⁺, 2.32 min (method 6). ¹H NMR (400 MHz, 1:1 mixture of CDCl₃ and MeOD, MeOD lock) δ ppm 6.65 (d, J=8.3 Hz, 2H), 5.94 (d, J=8.3 Hz, 2H), 4.04 (d, J=4.6 Hz, 1H), 1.94 (br. s., 6H), 1.86 (d, J=5.6 Hz, 4H), 1.31 (d, J=13.0 Hz, 1H), 1.13 (dd, J=12.7, 8.1 Hz, 1H), 0.90 (dd, J=17.1, 6.4 Hz, 1H), 0.84 (d, J=10.8 Hz, 1H), 0.58-0.45 (m, 2H), 0.44-0.20 (m, 9H), 0.20-−0.08 (m, 7H), −0.13 (d, J=12.7 Hz, 1H), −0.21 (s, 3H), −0.22 (s, 3H), −0.24 (s, 4H), −0.31 (s, 3H), −0.32-−0.36 (m, 6H), −0.76-−0.85 (m, 1H), −0.85-−0.93 (m, 1H), −0.93-−1.04 (m, 2H).

### Example B52

### Preparation of 4-((1R,3aS,5aR,5bR,7aR,11aS,11bR,13aR,13bR)-3a-((2-(1,1-dioxidothiomorpholino)ethyl)amino)-5a,5b,8,8,11a-pentamethyl-1-(1-methylcyclopropyl)-2,3,3a,4,5,5a,5b,6,7,7a,8,11,11a,11b,12,13,13a,13b-octadecahydro-1H-cyclopenta[a]chrysen-9-yl)benzoic acid.

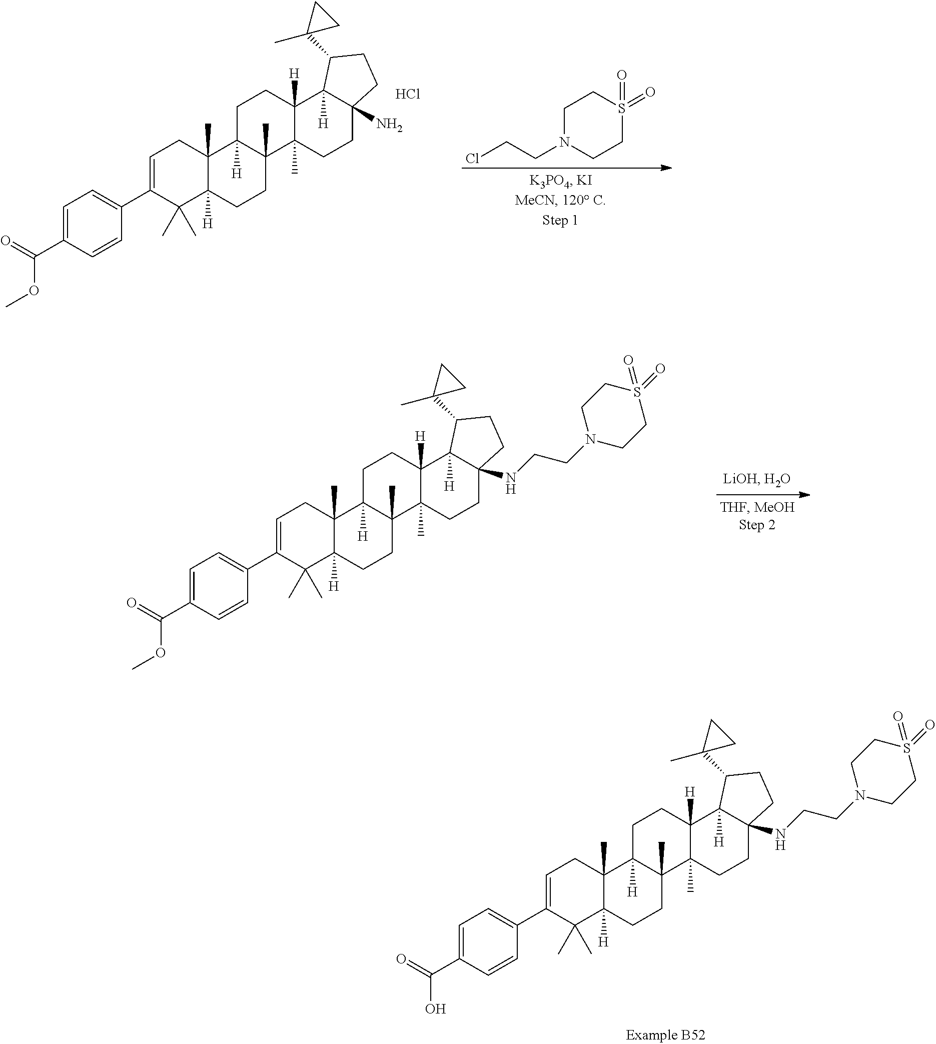

### Step 1. Preparation of methyl 4-((1R,3aS,5aR,5bR,7aR,11aS,11bR,13aR,13bR)-3a-((2-(1,1-dioxidothiomorpholino)ethyl)amino)-5a,5b,8,8,11a-pentamethyl-1-(1-methylcyclopropyl)-2,3,3a,4,5,5a,5b,6,7,7a,8,11,11a,11b,12,13,13a,13b-octadecahydro-1H-cyclopenta[a]chrysen-9-yl)benzoate.

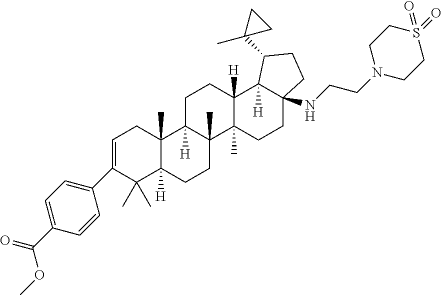

A pressure vessel containing methyl 4-((1R,3aS,5aR,5bR,7aR,11aS,11bR,13aR,13bR)-3a-amino-5a,5b,8,8,11a-pentamethyl-1-(1-methylcyclopropyl)-2,3,3a,4,5,5a,5b,6,7,7a,8,11,11a,11b,12,13,13a,13b-octadecahydro-1H-cyclopenta[a]chrysen-9-yl)benzoate hydrochloride (100 mg, 0.168 mmol), 4-(2-chloroethyl)thiomorpholine 1,1-dioxide hydrochloride (0.122 mg, 0.522 mmol) (prepared as described in WO1002045652), potassium phosphate, tribasic (0.157 g, 0.740 mmol) and potassium iodide (0.075 g, 0.454 mmol) in acetonitrile (3 mL) was sealed and heated to 120° C. for 64 h. The crude mixture was diluted with THF and filtered to remove solids. The filtrate was concentrated and product was isolated after reverse phase preparative HPLC purification using Prep HPLC Method 25 as a white solid (0.0982 mg, 61.6% yield) bis TFA salt. LCMS: m/e 719.7 (M+H)⁺, 2.10 min (method 6). ¹H NMR (400 MHz, CHLOROFORM-d) δ ppm 8.02-7.88 (m, J=8.3 Hz, 2H), 7.26-7.17 (m, J=8.3 Hz, 2H), 5.41-5.26 (m, 1H), 3.94 (s, 3H), 3.33-3.10 (m, 8H), 3.10-2.99 (m, 2H), 2.98-2.83 (m, 2H), 2.25-2.04 (m, 3H), 2.02-1.80 (m, 5H), 1.79-1.69 (m, 2H), 1.66-1.36 (m, 11H), 1.27 (d, J=9.0 Hz, 2H), 1.21 (s, 3H), 1.11 (s, 3H), 1.05 (s, 3H), 0.97 (br. s., 3H), 0.96 (s, 6H), 0.49-0.40 (m, 2H), 0.40-0.27 (m, 2H).

Step 2: A mixture of methyl 4-((1R,3aS,5aR,5bR,7aR,11aS,11bR,13aR,13bR)-3a-((2-(1,1-dioxidothiomorpholino)ethyl)amino)-5a,5b,8,8,11a-pentamethyl-1-(1-methylcyclopropyl)-2,3,3a,4,5,5a,5b,6,7,7a,8,11,11a,11b,12,13,13a,13b-octadecahydro-1H-cyclopenta[a]chrysen-9-yl)benzoate trifluoroacetic acid salt (0.098 g, 0.118 mmol) and 1M aqueous lithium hydroxide (0.588 mL, 0.588 mmol) with THF (1 mL) and MeOH (1 mL) in a sealed vial was heated to 75° C. for 1 h. The title compound, 4-((1R,3aS,5aR,5bR,7aR,11aS,11bR,13aR,13bR)-3a-((2-(1,1-dioxidothiomorpholino)ethyl)amino)-5a,5b,8,8,11a-pentamethyl-1-(1-methylcyclopropyl)-2,3,3a,4,5,5a,5b,6,7,7a,8,11,11a,11b,12,13,13a,13b-octadecahydro-1H-cyclopenta[a]chrysen-9-yl)benzoic acid was isolated after reverse phase preparative HPLC purification using Prep HPLC Method 24 as a white solid (89.9 mg, 79% yield) bis TFA salt. LCMS: m/e 705.8 (M+H)⁺, 1.93 min (method 6). ¹H NMR (400 MHz, 1:1 mixture of CDCl₃ and MeOD, MeOD lock) δ ppm 7.91 (d, J=8.3 Hz, 2H), 7.19 (d, J=8.1 Hz, 2H), 5.29 (d, J=4.4 Hz, 1H), 3.28-3.09 (m, 7H), 3.09-2.89 (m, 5H), 2.16 (dd, J=17.0, 6.2Hz, 1H), 2.07 (d, J=14.7 Hz, 3H), 1.98-1.89 (m, 1H), 1.89-1.78 (m, 3H), 1.78-1.64 (m, 2H), 1.63-1.36 (m, 12H), 1.35-1.25 (m, 2H), 1.17 (s, 3H), 1.10 (s, 3H), 1.03 (s, 3H), 0.95 (br. s., 3H), 0.94 (br. s., 3H), 0.93 (br. s., 3H), 0.47-0.37 (m, 2H), 0.37-0.24 (m, 2H).

### Example B53

### Preparation of 4-((1R,3aS,5aR,5bR,7aR,11aS,11bR,13aR,13bR)-5a,5b,8,8,11a-pentamethyl-1-(1-methylcyclopropyl)-3a-(2-(4-(methylsulfonyl)piperidin-1-yl)ethylamino)-2,3,3a,4,5,5a,5b,6,7,7a,8,11,11a,11b,12,13,13a,13b-octadecahydro-1H-cyclopenta[a]chrysen-9-yl)benzoic acid.

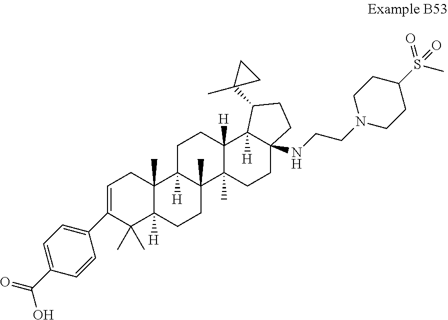

The title compound was prepared by the same two step process employed to prepare 4-((1R,3aS,5aR,5bR,7aR,11aS,11bR,13aR,13bR)-3a-((2-(1,1-dioxidothiomorpholino)ethyl)amino)-5a,5b,8,8,11a-pentamethyl-1-(1-methylcyclopropyl)-2,3,3a,4,5,5a,5b,6,7,7a,8,11,11a,11b,12,13,13a,13b-octadecahydro-1H-cyclopenta[a]chrysen-9-yl)benzoic acid, except 1-(2-chloroethyl)-4-(methylsulfonyl)piperidine (0.152 g, 0.673 mmol) was used in place of 4-(2-chloroethyl)thiomorpholine 1,1-dioxide hydrochloride in Step 1. The title compound was isolated after reverse phase preparative HPLC purification of the Step 2 reaction mixture using Prep HPLC Method 24 as a white solid (70.5 mg, 71.5% yield) bis TFA salt.

LCMS: m/e 733.6 (M+H)⁺, 2.28 min (method 5). ¹H NMR (400 MHz, 1:1 mixture of CDCl₃ and MeOD, MeOD lock) δ ppm 7.97-7.87 (m, J=8.3 Hz, 2H), 7.25-7.15 (m, J=8.6 Hz, 2H), 5.31 (dd, J=6.0, 1.6 Hz, 1H), 3.26 (t, J=11.7 Hz, 2H), 3.21-3.16 (m, 2H), 3.16-3.02 (m, 2H), 2.94 (s, 3H), 2.92-2.83 (m, 1H), 2.56 (t, J=11.2Hz, 1H), 2.42 (t, J=12.0 Hz, 1H), 2.27-2.13 (m, 3H), 2.13-1.94 (m, 5H), 1.94-1.83 (m, 3H), 1.82-1.66 (m, 3H), 1.63-1.38 (m, 11H), 1.35 (d, J=12.2Hz, 1H), 1.31-1.23 (m, 1H), 1.20 (s, 3H), 1.11 (s, 3H), 1.04 (s, 3H), 0.98 (s, 3H), 0.96 (s, 3H), 0.94 (s, 3H), 0.50-0.39 (m, 2H), 0.39-0.26 (m, 2H).

### Example B54

### Preparation of 4-((1R,3aS,5aR,5bR,7aS,9S,11aS,11bR,13aR,13bR)-3a-amino-5a,5b,8,8,11a-pentamethyl-1-(1-methylcyclopropyl)icosahydro-1H-cyclopenta[a]chrysen-9-yl)benzoic acid.

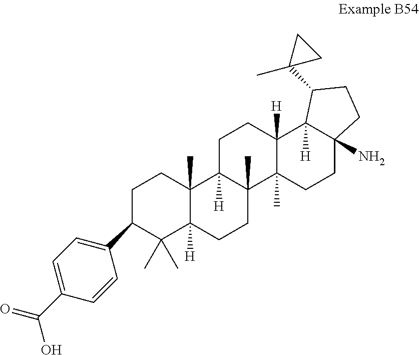

A mixture of 4-((1R,3aS,5aR,5bR,7aR,11aS,11bR,13aR,13bR)-3a-amino-5a,5b,8,8,11a-pentamethyl-1-(1-methylcyclopropyl)-2,3,3a,4,5,5a,5b,6,7,7a,8,11,11a,11b,12,13,13a,13b-octadecahydro-1H-cyclopenta[a]chrysen-9-yl)benzoic acid (0.036 g, 0.055 mmol) and ethyl acetate (2 mL) was blanketed with nitrogen gas, then 10% palladium on carbon (0.023 g, 0.022 mmol) was added. To the flask was fitted a balloon of hydrogen gas, and the mixture was stirred rapidly under hydrogen atmosphere for 18 h. The mixture was filtered to remove catalyst and concentrated in vacuo. The title compound was isolated after reverse phase preparative HPLC purification of the reaction mixture using Prep HPLC Method 24 as a white solid (13.1 mg, 36.4% yield) mono TFA salt. LCMS: m/e 546.7 (M+H)⁺, 1.95 min (method 6). ¹H NMR (400 MHz, 1:1 mixture of CDCl₃ and MeOD, MeOD lock) δ ppm 7.89 (d, J=8.3 Hz, 2H), 7.23 (d, J=8.3 Hz, 2H), 2.42 (dd, J=13.1, 2.8 Hz, 1H), 2.19-2.08 (m, 2H), 2.04-1.97 (m, 1H), 1.92-1.71 (m, 5H), 1.66-1.40 (m, 11H), 1.39-1.28 (m, 3H), 1.24 (br. s., 1H), 1.10 (s, 5H), 1.06 (s, 4H), 0.99 (s, 3H), 0.95 (s, 4H), 0.76 (s, 3H), 0.70 (s, 3H), 0.49-0.41 (m, 1H), 0.41-0.34 (m, 1H), 0.34-0.26 (m, 2H).

### Example B55

### Preparation of 4-((1R,3aS,5aR,5bR,7aS,9S,11aS,11bR,13aR,13bR)-5a,5b,8,8,11a-pentamethyl-3a-(methylamino)-1-(1-methylcyclopropypicosahydro-1H-cyclopenta[a]chrysen-9-yl)benzoic acid.

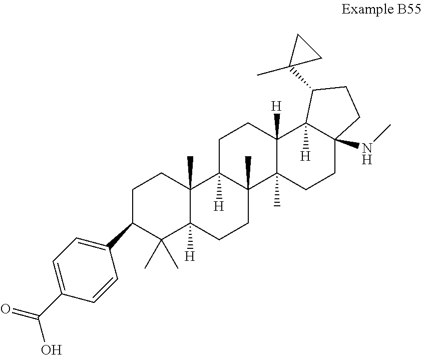

The title compound was prepared following a similar procedure as described for the synthesis of 4-((1R,3aS,5aR,5bR,7aS,9S,11aS,11bR,13aR,13bR)-3a-amino-5a,5b,8,8,11a-pentamethyl-1-(1-methylcyclopropyl)icosahydro-1H-cyclopenta[a]chrysen-9-yl)benzoic acid, except 4-((1R,3aS,5aR,5bR,7aR,11aS,11bR,13aR,13bR)-5a,5b,8,8,11a-pentamethyl-3a-(methylamino)-1-(1-methylcyclopropyl)-2,3,3a,4,5,5a,5b,6,7,7a,8,11,11a,11b,12,13,13a,13b-octadecahydro-1H-cyclopenta[a]chrysen-9-yl)benzoic acid (0.034 g, 0.051 mmol) was used in place of 4-((1R,3aS,5aR,5bR,7aR,11aS,11bR,13aR,13bR)-3a-amino-5a,5b,8,8,11a-pentamethyl-1-(1-methylcyclopropyl)-2,3,3a,4,5,5a,5b,6,7,7a,8,11,11a,11b,12,13,13a,13b-octadecahydro-1H-cyclopenta[a]chrysen-9-yl)benzoic acid. Purification of the crude mixture by reverse phase preparative HPLC using Prep HPLC Method 23 gave the title compound as a white solid (25.5 mg, 63.6% yield) mono TFA salt. LCMS: m/e 560.8 (M+H)⁺, 2.14 min (method 6). ¹H NMR (400 MHz, 1:1 mixture of CDCl₃ and MeOD, MeOD lock) δ ppm 7.89 (d, J=8.3 Hz, 2H), 7.23 (d, J=8.3 Hz, 2H), 2.56 (s, 3H), 2.42 (dd, J=13.2, 2.9 Hz, 1H), 2.17-2.09 (m, 2H), 2.05-1.77 (m, 6H), 1.73-1.54 (m, 4H), 1.54-1.28 (m, 10H), 1.21-1.13 (m, 1H), 1.11 (s, 3H), 1.09 (s, 3H), 1.06 (br. s., 1H), 0.99 (s, 3H), 0.96 (s, 4H), 0.76 (s, 3H), 0.70 (s, 3H), 0.50-0.42 (m, 1H), 0.42-0.35 (m, 1H), 0.35-0.25 (m, 2H).

### Example B56

### Preparation of 4-((1R,3aS,5aR,5bR,7aR,11aS,11bR,13aR,13bR)-5a,5b,8,8,11a-pentamethyl-1-(1-methylcyclopropyl)-3a-(2-(4-(methylsulfonyl)piperazin-1-yl)ethylamino)-2,3,3a,4,5,5a,5b,6,7,7a,8,11,11a,11b,12,13,13a,13b-octadecahydro-1H-cyclopenta[a]chrysen-9-yl)benzoic acid

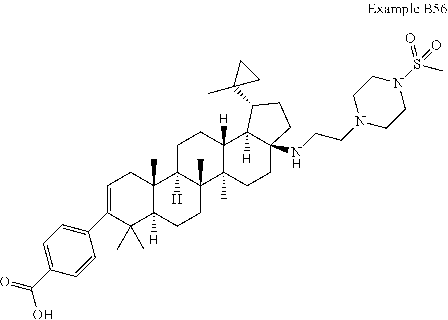

The title compound was prepared by the same two step process employed to prepare 4-((1R,3aS,5aR,5bR,7aR,11aS,11bR,13aR,13bR)-3a-((2-(1,1-dioxidothiomorpholino)ethyl)amino)-5a,5b,8,8,11a-pentamethyl-1-(1-methylcyclopropyl)-2,3,3a,4,5,5a,5b,6,7,7a,8,11,11a,11b,12,13,13a,13b-octadecahydro-1H-cyclopenta[a]chrysen-9-yl)benzoic acid, except 1-(2-chloroethyl)-4-(methylsulfonyl)piperazine (0.118 g, 0.522 mmol) was used in place of 4-(2-chloroethyl)thiomorpholine 1,1-dioxide hydrochloride in Step 1. The title compound was isolated after reverse phase preparative HPLC purification of the Step 2 reaction mixture using Prep HPLC Method 4 as a white solid (58.7 mg, 59.2% yield) bis TFA salt. LCMS: m/e 734.4 (M+H)⁺, 2.30 min (method 5). ¹H NMR (400 MHz, 1:1 mixture of CDCl₃ and MeOD, MeOD lock) δ ppm 7.93 (d, J=8.3 Hz, 2H), 7.21 (d, J=8.3 Hz, 2H), 5.32 (d, J=4.6 Hz, 1H), 3.31-3.21 (m, 2H), 3.21-3.10 (m, 2H), 3.10-2.99 (m, 2H), 2.88 (s, 3H), 2.83-2.66 (m, 6H), 2.18 (dd, J=17.0, 6.2Hz, 1H), 2.14-2.02 (m, 2H), 2.02-1.82 (m, 3H), 1.82-1.68 (m, 2H), 1.68-1.39 (m, 12H), 1.37 (d, J=11.7 Hz, 1H), 1.33-1.23 (m, 2H), 1.21 (s, 3H), 1.13 (s, 3H), 1.05 (s, 3H), 0.98 (s, 3H), 0.96 (s, 6H), 0.51-0.42 (m, 2H), 0.42-0.27 (m, 2H).

### Biology Data for the Examples

“μM” means micromolar;
“mL” means milliliter;
“μl” means microliter;
“mg” means milligram;
“μg” means microgram;

The materials and experimental procedures used to obtain the results reported in Table 1 are described below.

### HIV Cell Culture Assay

MT-2 cells and 293T cells were obtained from the NIH AIDS Research and Reference Reagent Program. MT-2 cells were propagated in RPMI 1640 media supplemented with 10% heat inactivated fetal bovine serum, 100 pg/ml penicillin G and up to 100 units/ml streptomycin. The 293T cells were propagated in DMEM media supplemented with 10% heat inactivated fetal bovine serum (FBS), 100 units/ml penicillin G and 100 g/ml streptomycin. The proviral DNA clone of NL₄₋₃ was obtained from the NIH AIDS Research and Reference Reagent Program. A recombinant NL₄₋₃ virus, in which a section of the nef gene from NL4-3 was replaced with the Renilla luciferase gene, was used as a reference virus. In addition, residue Gag P373 was converted to P373S. Briefly, the recombinant virus was prepared by transfection of the altered proviral clone of NL₄₋₃. Transfections were performed in 293T cells using LipofectAMINE PLUS from Invitrogen (Carlsbad, Calif.), according to manufacturer's instruction. The virus was titered in MT-2 cells using luciferase enzyme activity as a marker. Luciferase was quantitated using the Dual Luciferase kit from Promega (Madison, Wis.), with modifications to the manufacturer's protocol. The diluted Passive Lysis solution was pre-mixed with the re-suspended Luciferase Assay Reagent and the re-suspended Stop & Glo Substrate (2:1:1 ratio). Fifty (50) μL of the mixture was added to each aspirated well on assay plates and luciferase activity was measured immediately on a Wallac TriLux (Perkin-Elmer). Antiviral activities of inhibitors toward the recombinant virus were quantified by measuring luciferase activity in cells infected for 4-5 days with NLRluc recombinants in the presence serial dilutions of the inhibitor. The EC₅₀ data for the compounds is shown in Table 1.

TABLE 1

| Example # | Structure | EC50 μM |
|---|---|---|
| 1 | 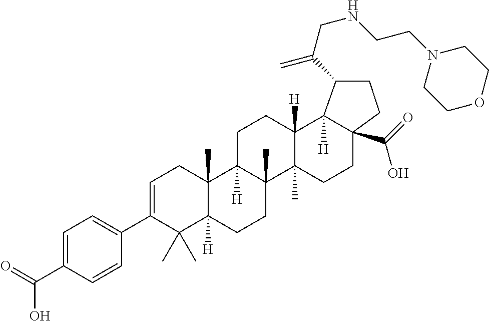 | 0.04 |
| 2 | 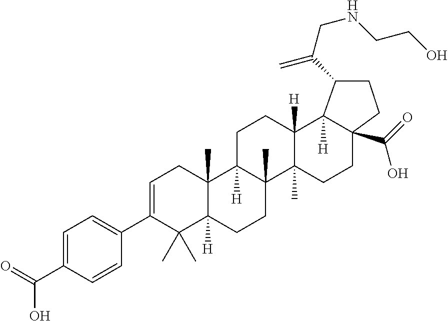 | 0.14 |
| 3 | 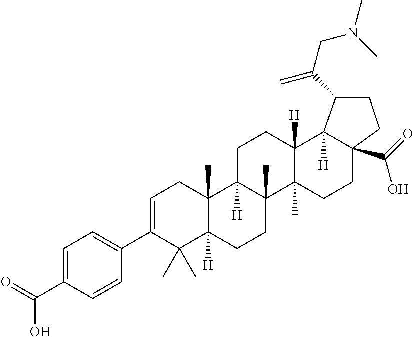 | 0.10 |
| 4 | 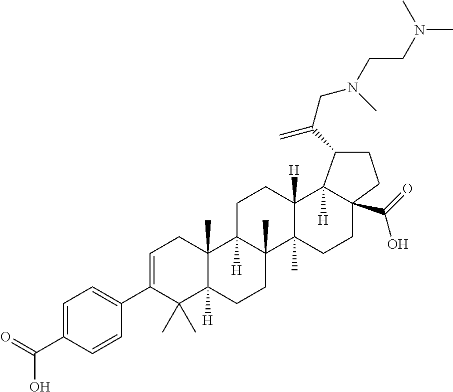 | 1.42E−03 |
| 5 | 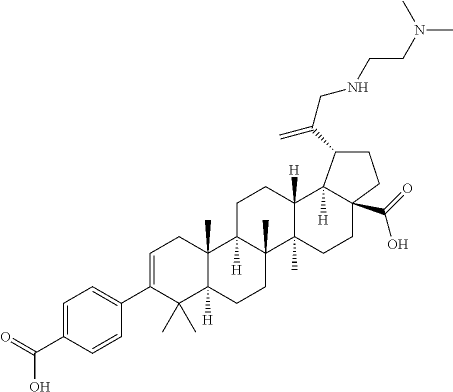 | 0.16 |
| 6 | 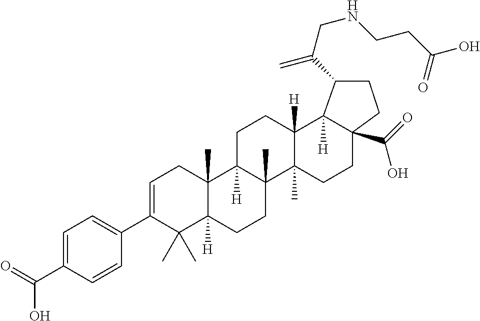 | 4.00 |
| 7 | 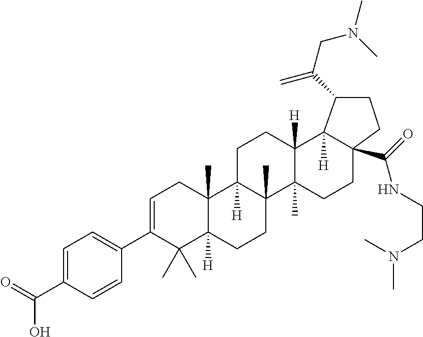 | 5.40E−04 |
| 8 | 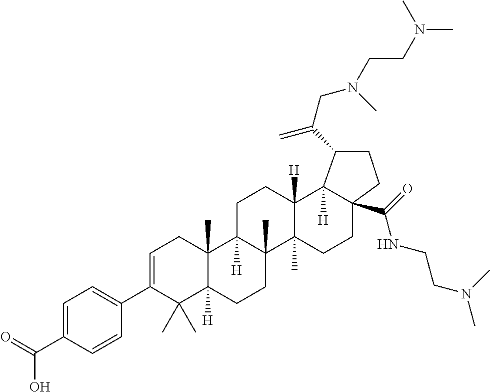 | 1.49E−03 |
| 9 | 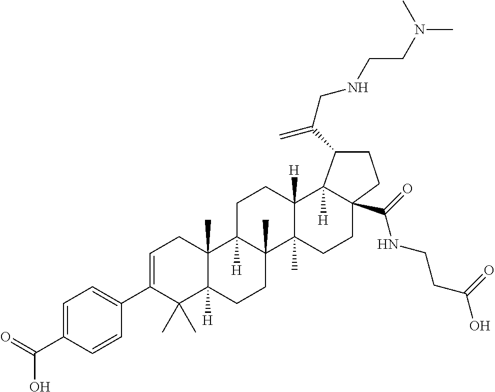 | 0.09 |
| 10 | 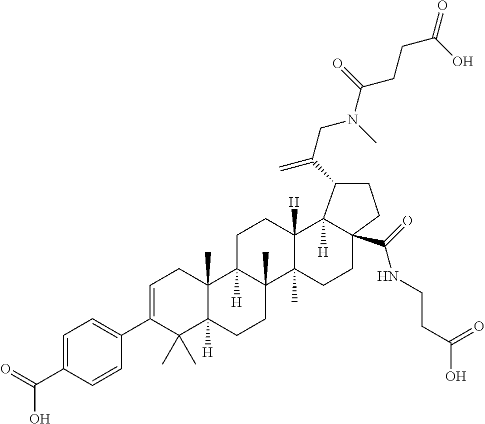 | 2.00 |
| 11 | 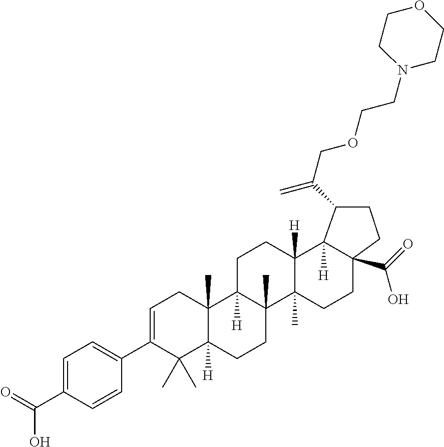 | 0.02 |
| 12 | 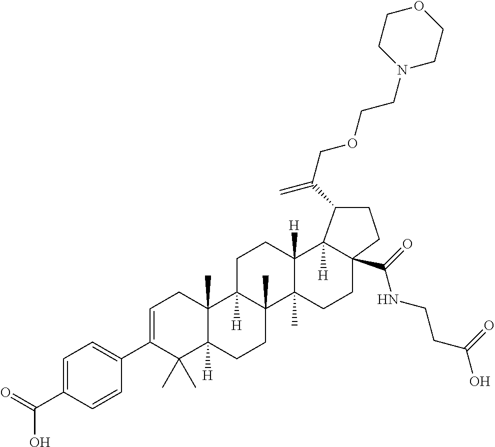 | 0.14 |
| 13 | 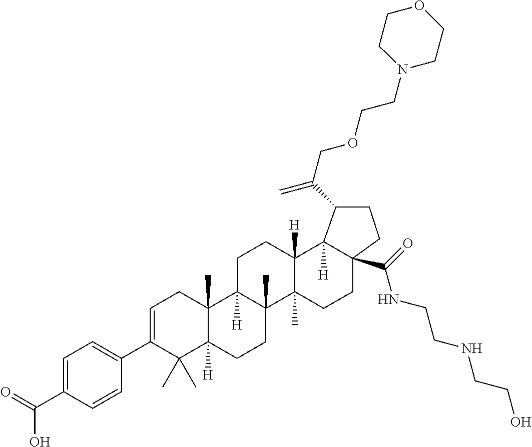 | 1.97E−03 |
| 14 | 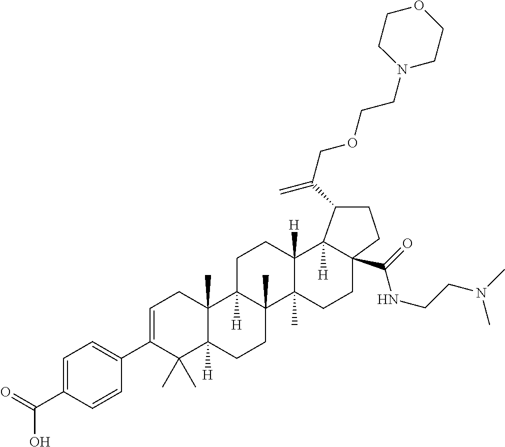 | 7.20E−04 |
| 15 | 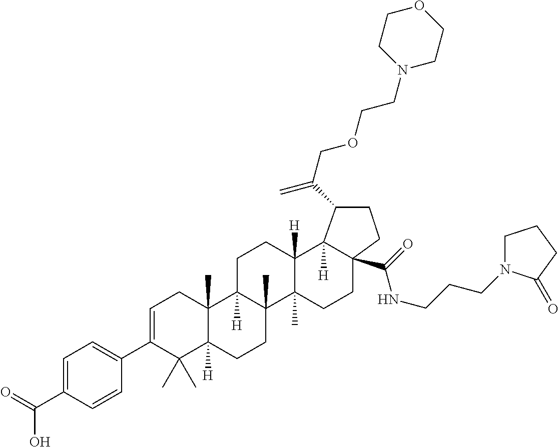 | 1.83E−03 |
| 16 | 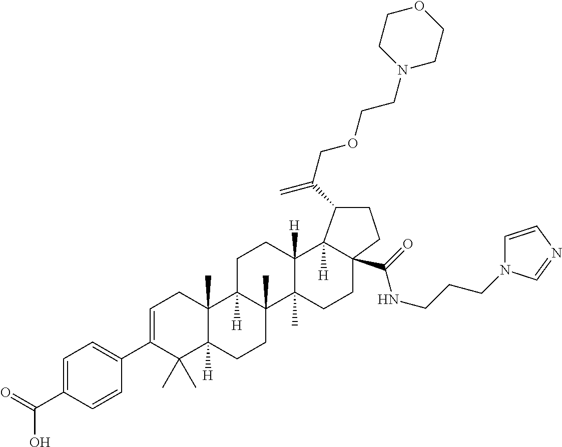 | 2.48E−03 |
| 17 | 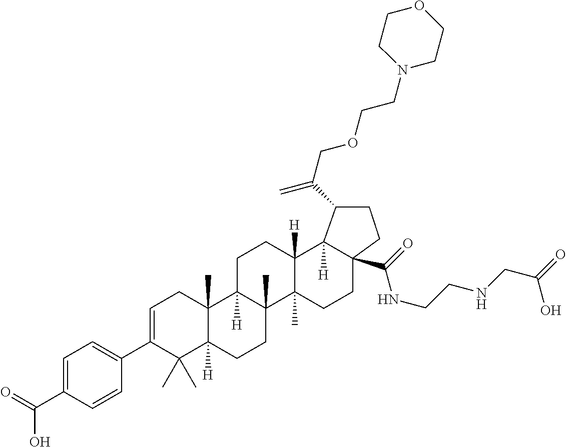 | 0.08 |
| 18 | 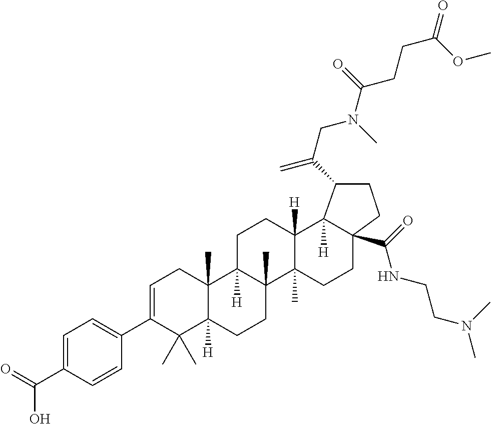 | 1.87E−03 |
| 19 | 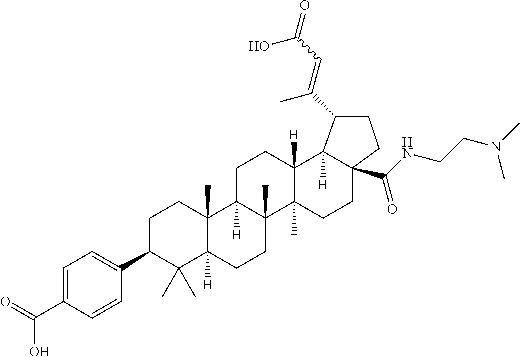 | 0.02 |
| 20 | 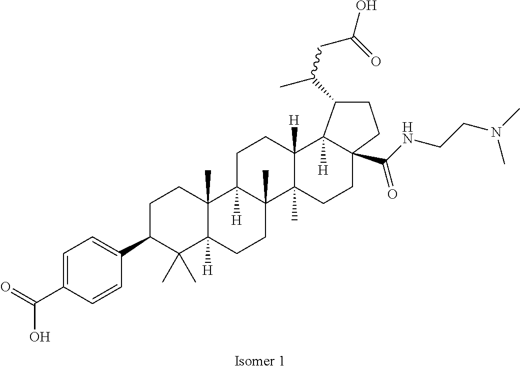 | 0.03 |
| 21 | 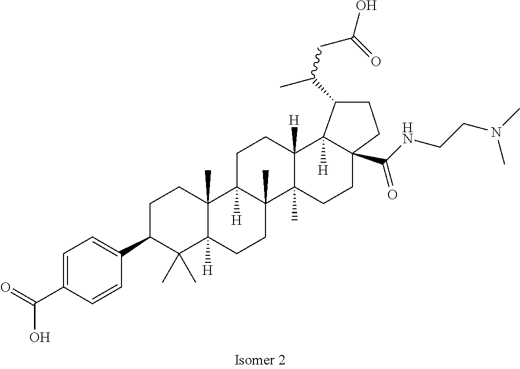 | 0.04 |
| 22 | 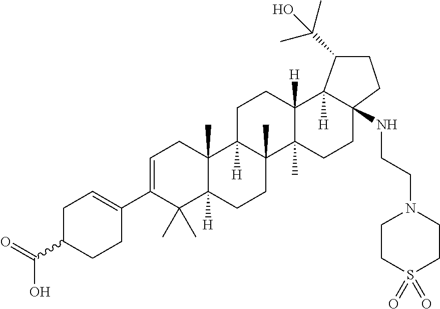 | 5.19E−04 |
| 23 | 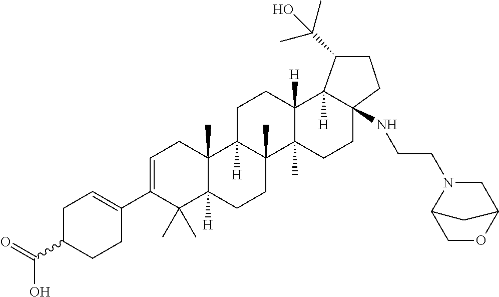 | 7.79E−04 |
| A1 | 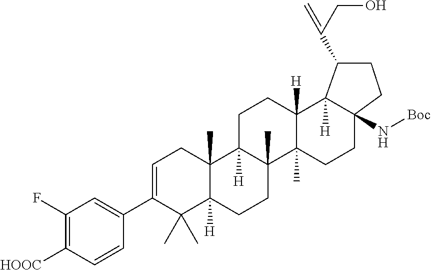 | 0.02 |
| A2 | 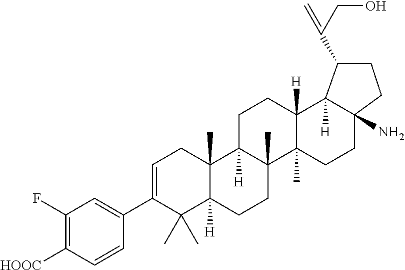 | 0.01 |
| A3 | 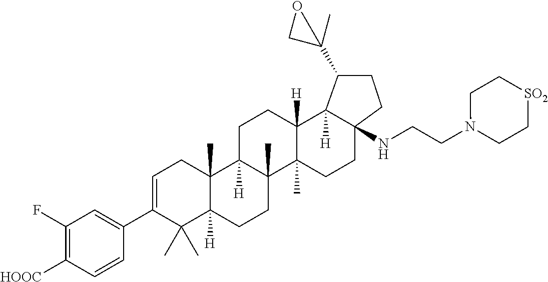 | 0.01 |
| A4 | 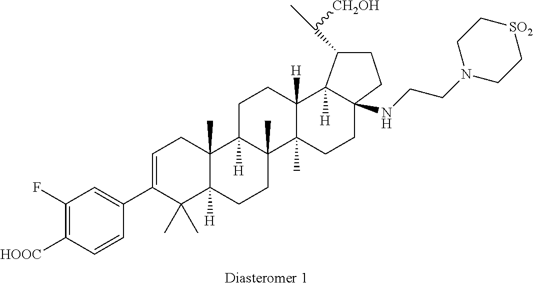 | 2.34E−03 |
| A5 | 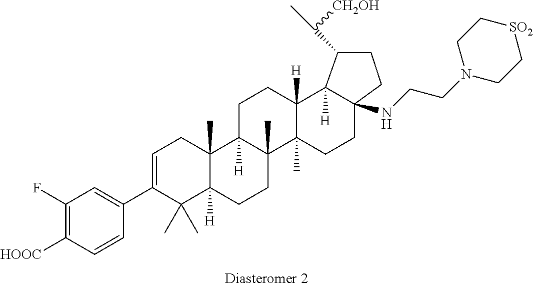 | 6.89E−03 |
| A6 | 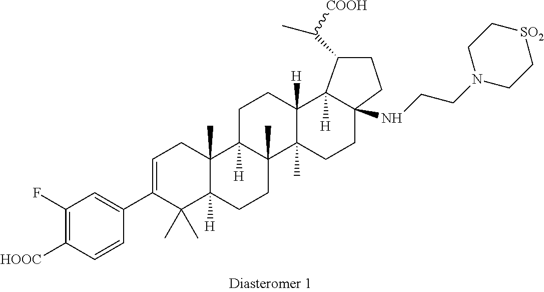 | 0.39 |
| A7 | 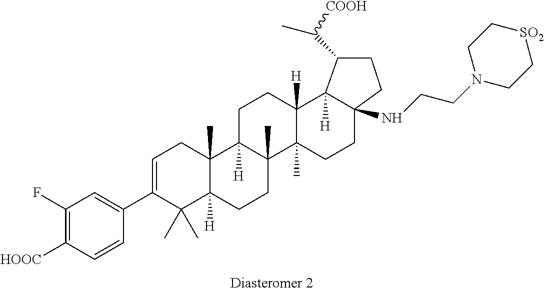 | 0.11 |
| A8 | 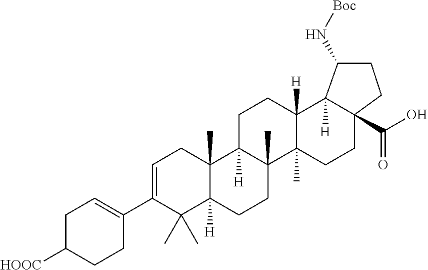 | 6.56E−03 |
| A9 | 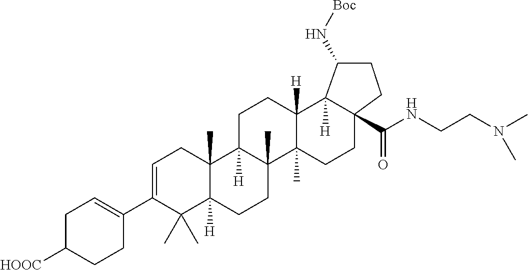 | 1.15E−03 |
| B1 | 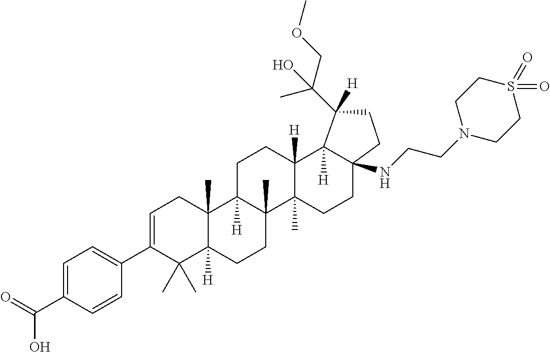 | 1.19E−03 |
| B2 | 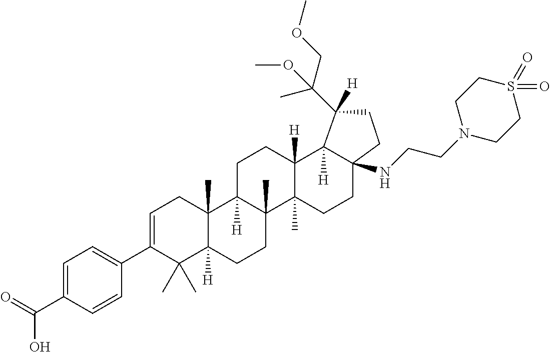 | 1.05E−03 |
| B3 | 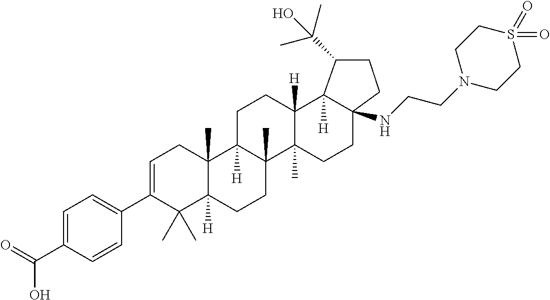 | 1.30E−03 |
| B4 | 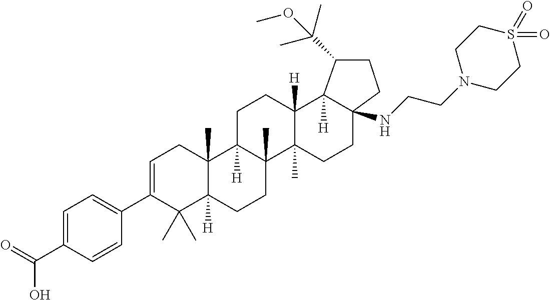 | 2.06E−03 |
| B5 | 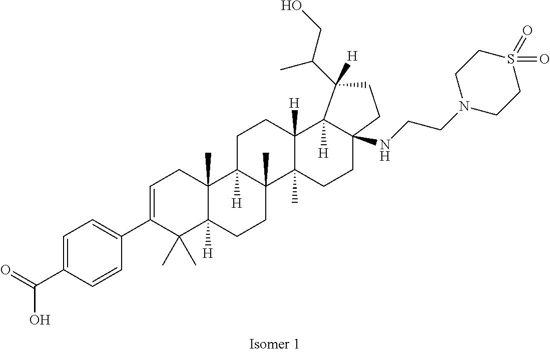 | 1.15E−03 |
| B6 | 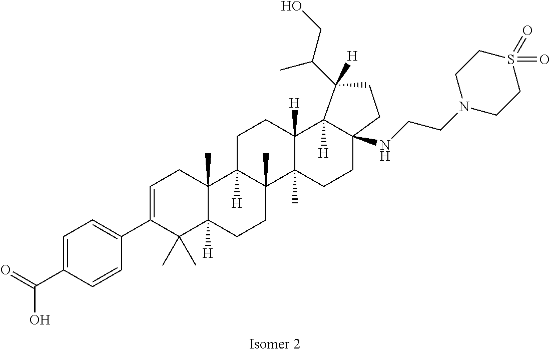 | 3.75E−03 |
| B7 | 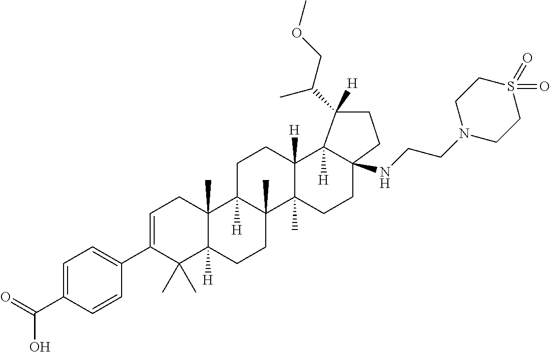 | 9.45E−04 |
| B8 | 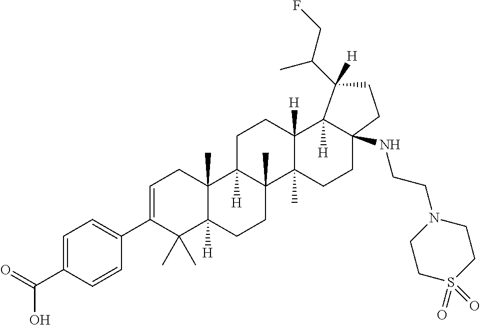 | 2.51E−03 |
| B9 | 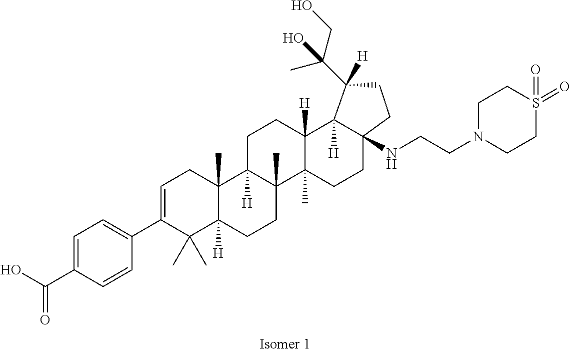 | 2.15E−03 |
| B10 | 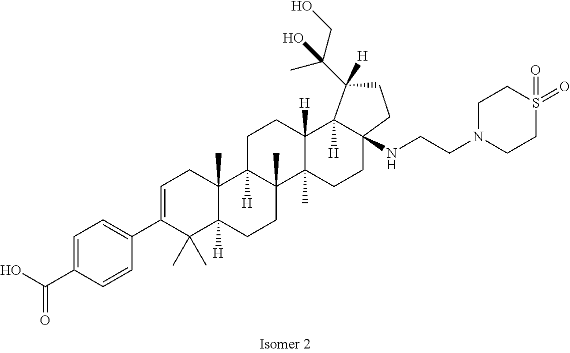 | 6.67E−03 |
| B11 | 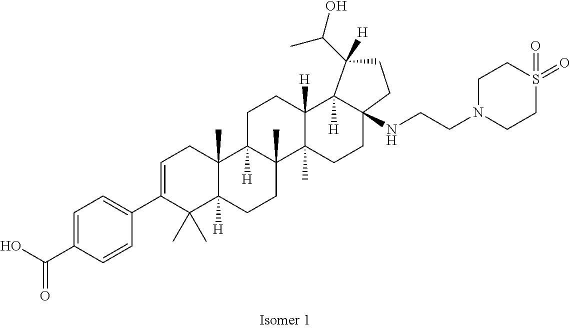 | 2.18E−03 |
| B12 | 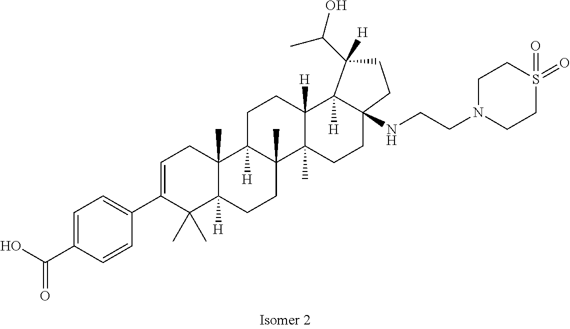 | 3.95E−03 |
| B13 | 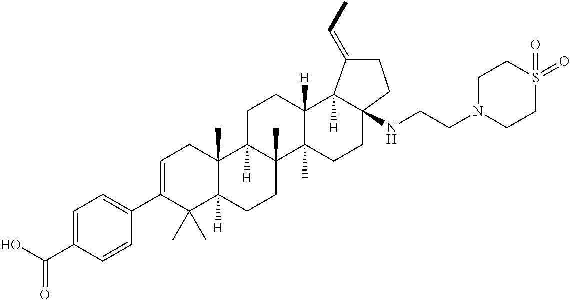 | 0.01 |
| B14 | 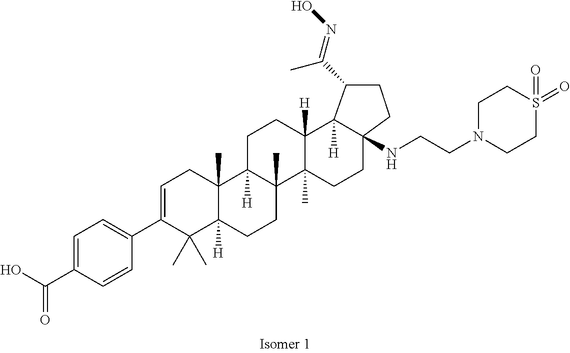 | 3.75E−03 |
| B15 | 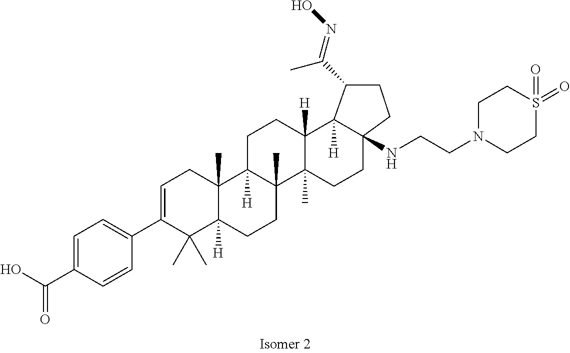 | |
| B16 | 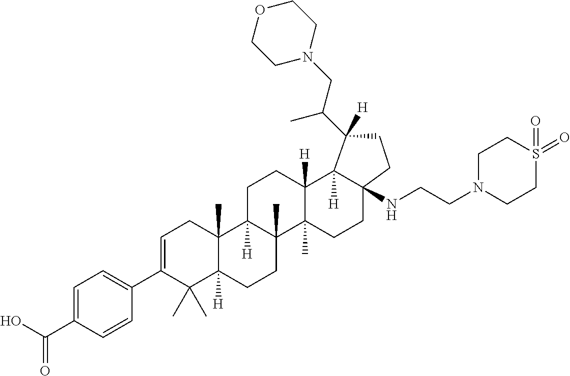 | 8.27E−04 |
| B17 | 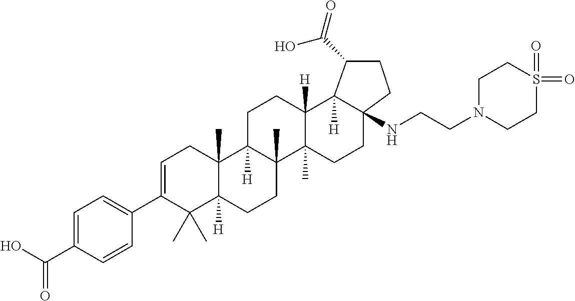 | 2.07 |
| B18 | 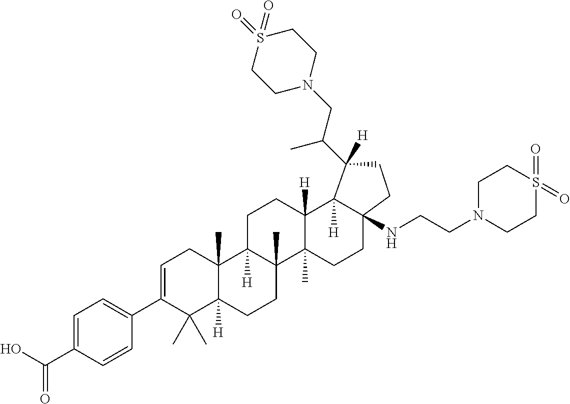 | 1.57E−03 |
| B19 | 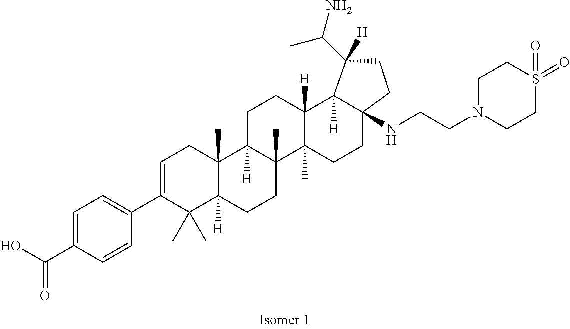 | 0.03 |
| B20 | 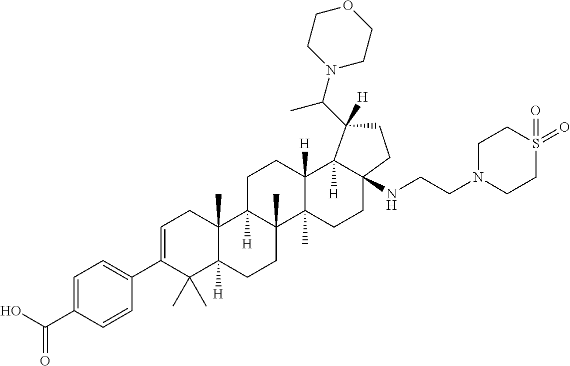 | 2.05E−03 |
| B21 | 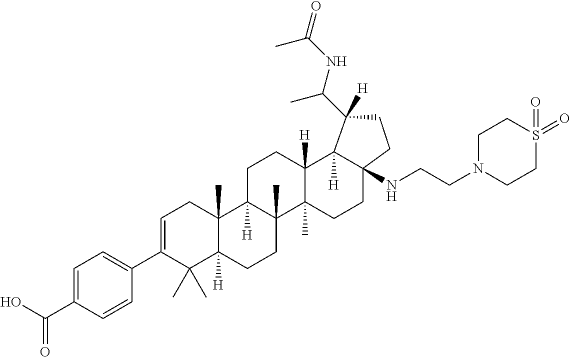 | 8.18E−03 |
| B22 | 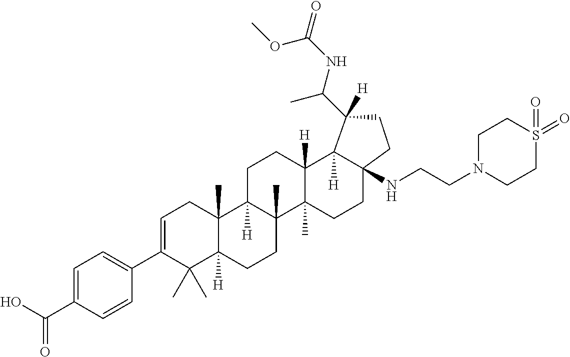 | 1.02E−03 |
| B23 | 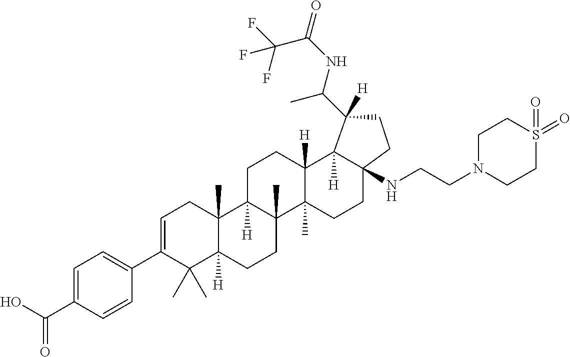 | 1.16E−03 |
| B24 | 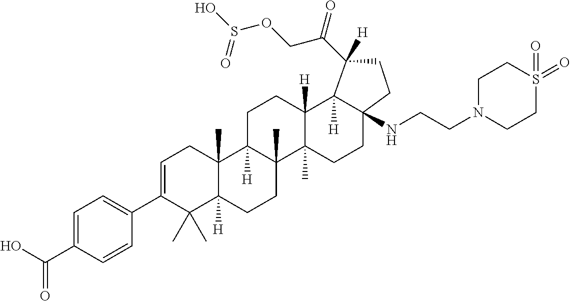 | 1.00 |
| B25 | 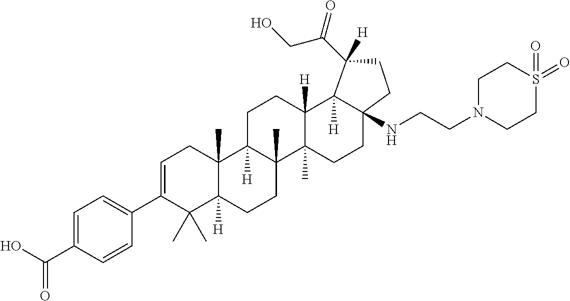 | 0.11 |
| B26 | 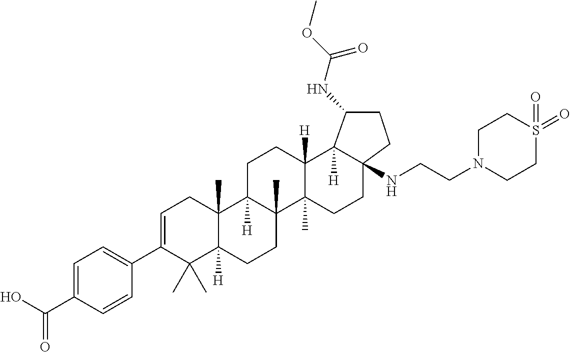 | 2.85E−03 |
| B27 | 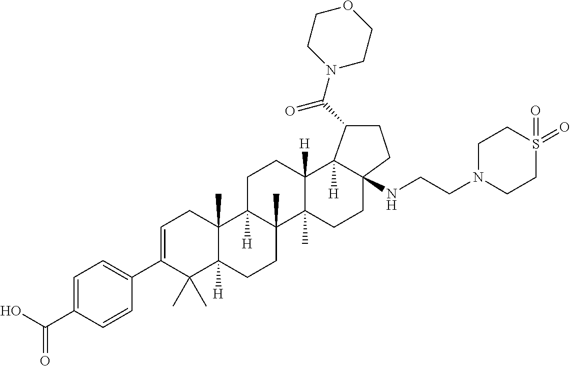 | 8.11E−03 |
| B28 | 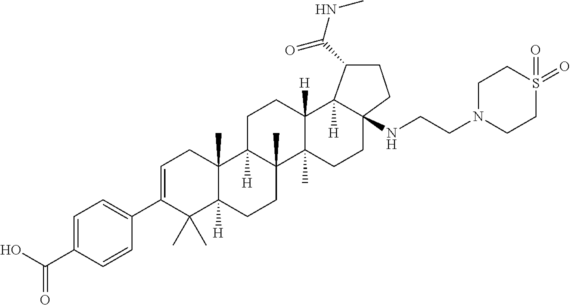 | 0.11 |
| B29 | 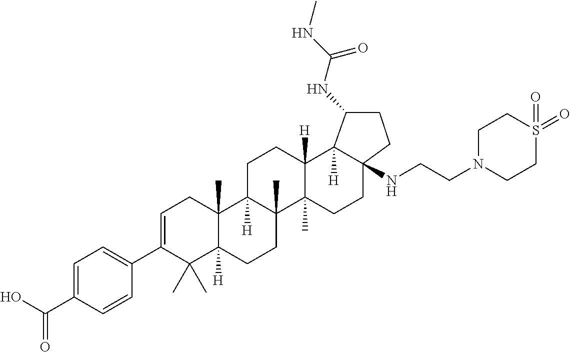 | 0.03 |
| B30 | 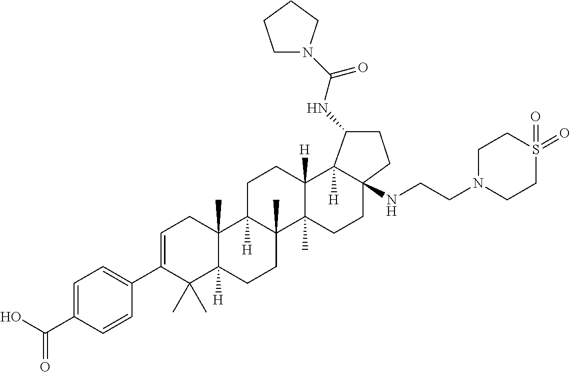 | 2.96E−03 |
| B31 | 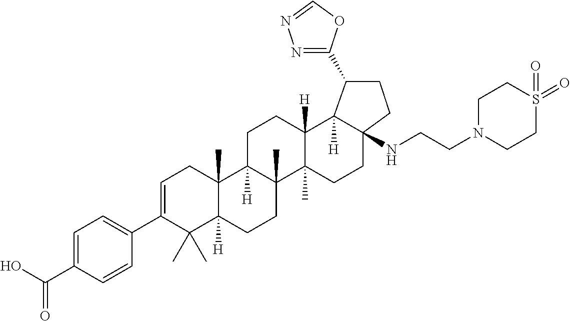 | 0.03 |
| B32 | 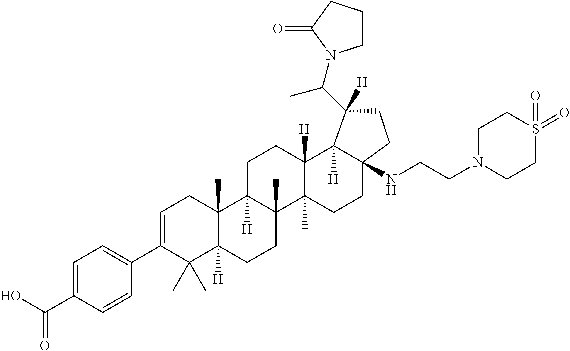 | 2.84E−03 |
| B33 | 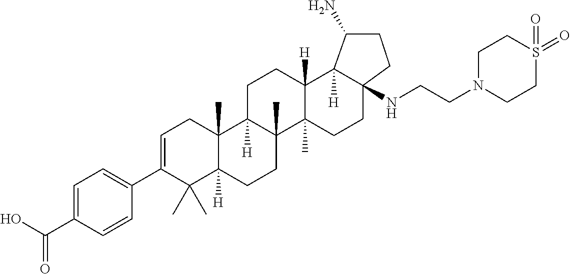 | 0.13 |
| B34 | 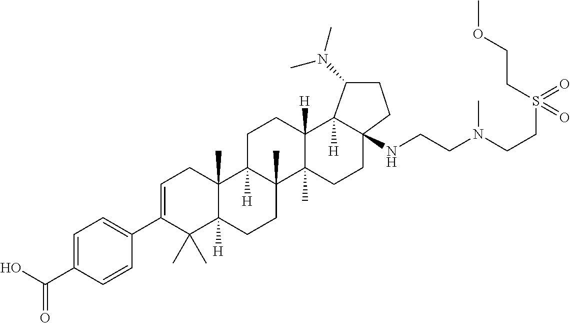 | 0.02 |
| B35 | 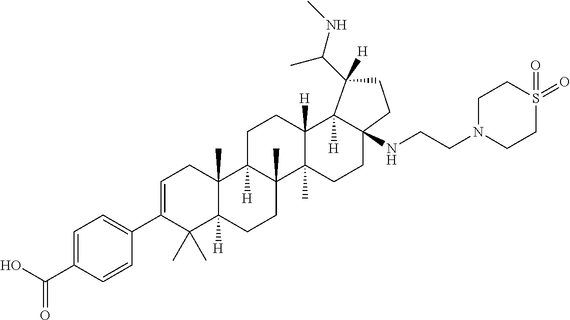 | 4.24E−03 |
| B36 | 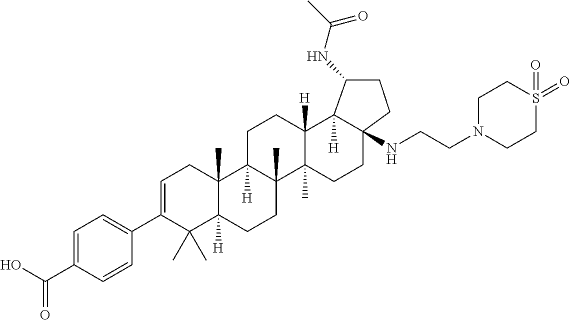 | 6.29E−03 |
| B37 | 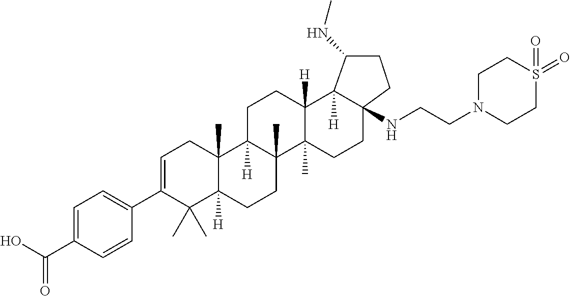 | 0.04 |
| B38 | 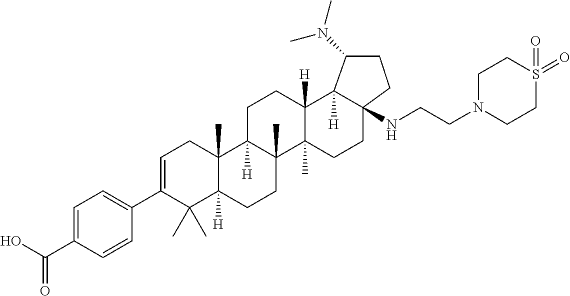 | 0.01 |
| B39 | 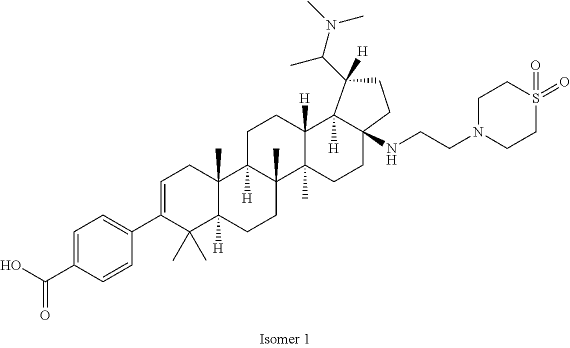 | 4.33E−03 |
| B40 | 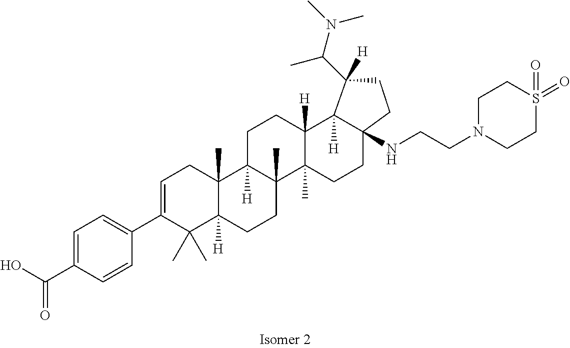 | 0.04 |
| B41 | 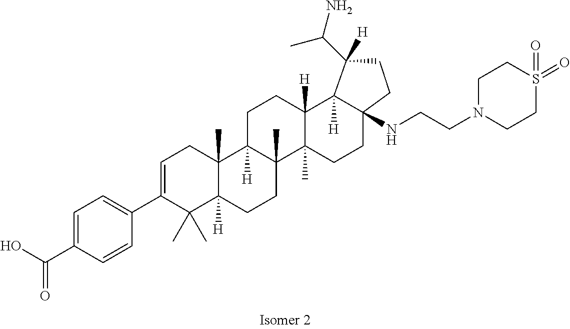 | 0.04 |
| B42 | 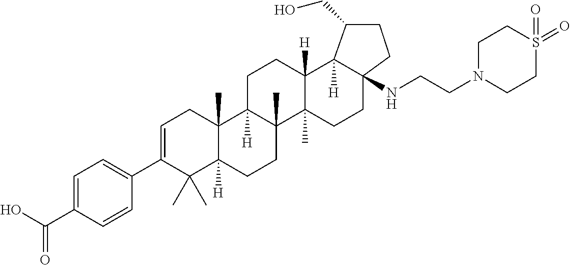 | 0.04 |
| B43 | 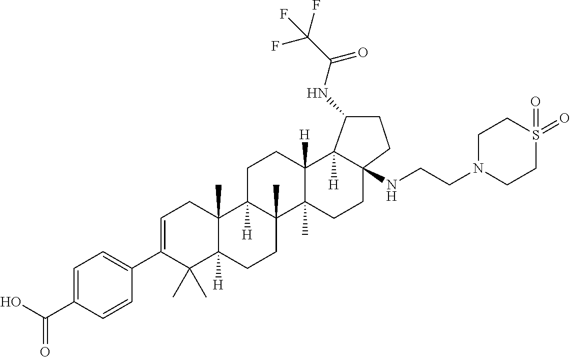 | 2.12E−03 |
| B44 | 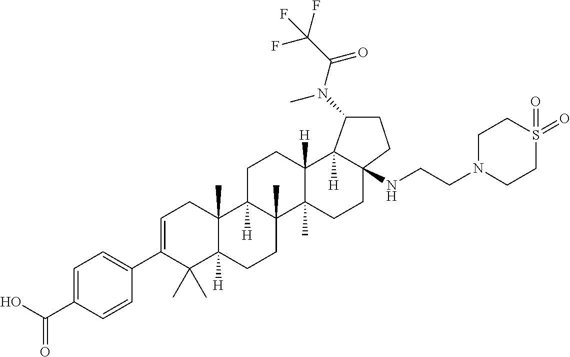 | 5.92E−03 |
| B45 | 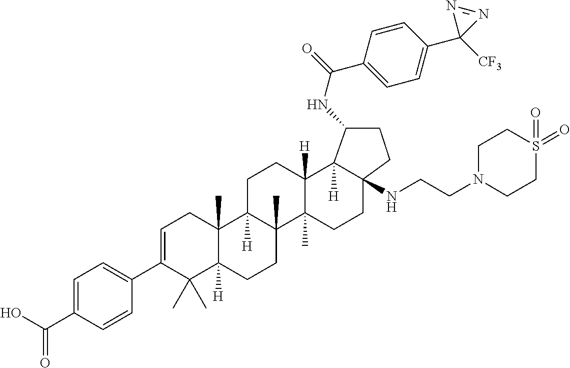 | 0.05 |
| B46 | 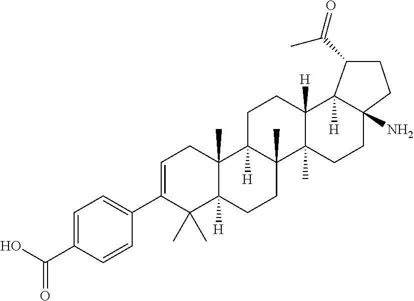 | 0.29 |
| B47 | 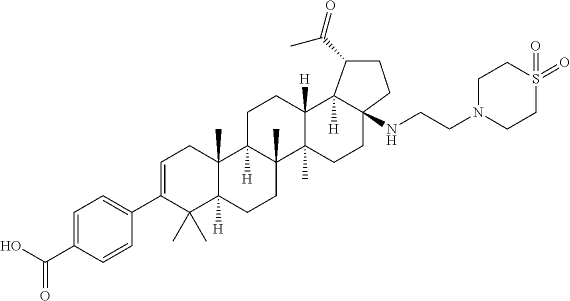 | 0.01 |
| B48 | 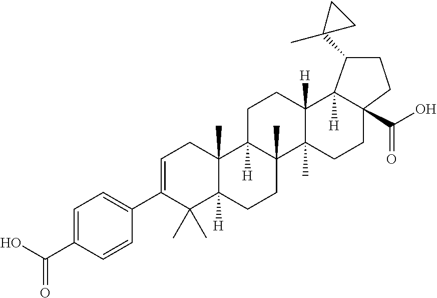 | 0.01 |
| B49 | 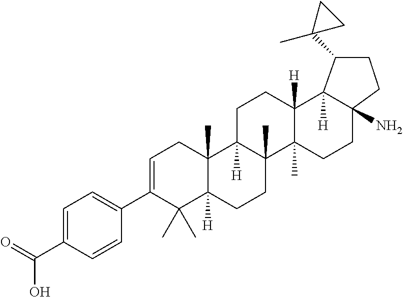 | 9.85E−04 |
| B50 | 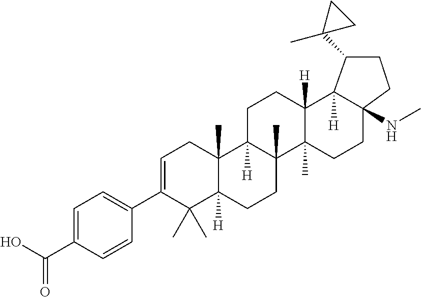 | 1.36E−03 |
| B51 | 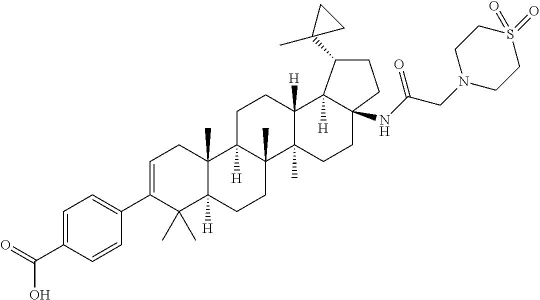 | 1.10E−03 |
| B52 | 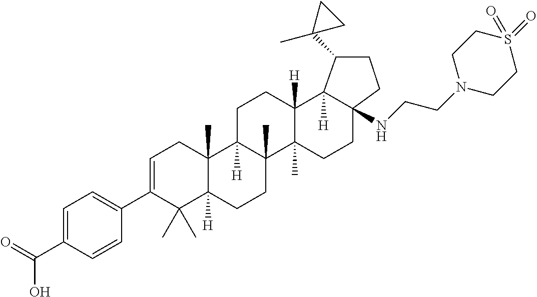 | 9.24E−04 |
| B53 | 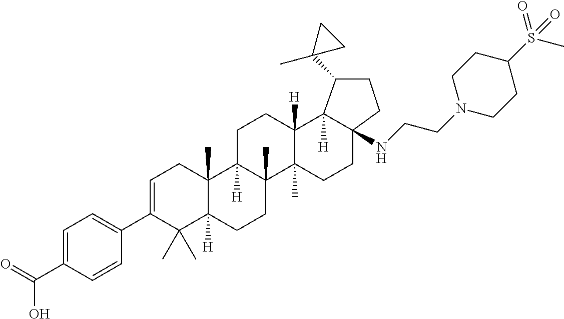 | 1.03E−03 |
| B54 | 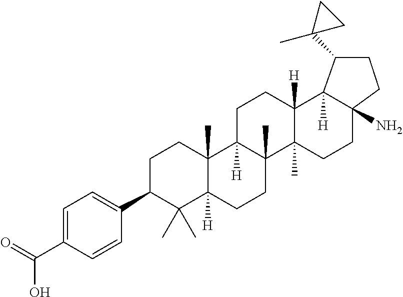 | 5.77E−04 |
| B55 | 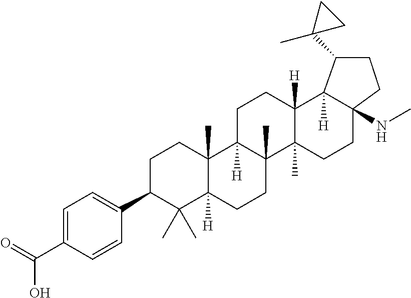 | 1.51E−03 |
| B56 | 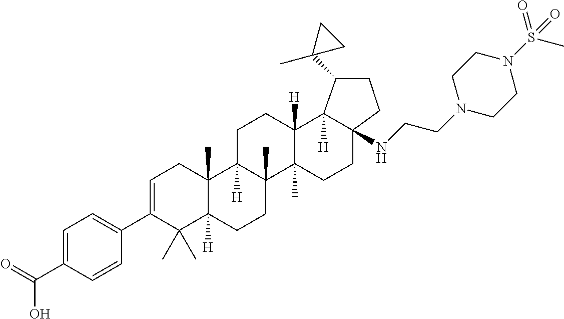 | 2.51E−03 |

The foregoing description is merely illustrative and should not be understood to limit the scope or underlying principles of the invention in any way. Indeed, various modifications of the invention, in addition to those shown and described herein, will become apparent to those skilled in the art from the following examples and the foregoing description. Such modifications are also intended to fall within the scope of the appended claims.

What is claimed is:

1. A compound, including pharmaceutically acceptable salts thereof, which is selected from the group of:
a compound of formula I 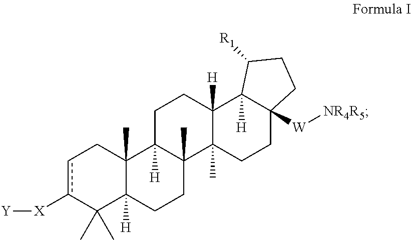
and
a compound of formula II 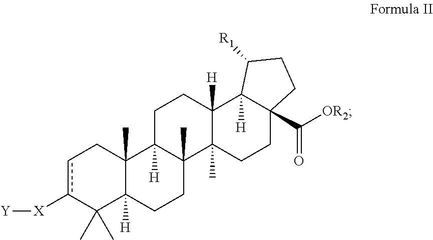
wherein X is selected from the group of phenyl, heteroaryl ring, C₄₋₈ cycloalkyl, C₄₋₈ cycloalkenyl, C₄₋₉ spirocycloalkyl, C₄₋₉ spirocycloalkenyl, C₄₋₈ oxacycloalkyl, C₄₋₈ dioxacycloalkyl, C₆₋₈ oxacycloalkenyl, C₆₋₈ dioxacycloalkenyl, C₆ cyclodialkenyl, C₆ oxacyclodialkenyl, C₆₋₉ oxaspirocycloalkyl and C₆₋₉ oxaspirocycloalkenyl ring;
and further wherein X is substituted with A, wherein A is at least one member selected from the group of —H, -halo, -hydroxyl, —C₁₋₆ alkyl, —C₁₋₆ alkoxy, —C₁₋₆ alkyl-Q₁, -alkylsubstituted C₁₋₆ alkyl-Q₁, —CN, —CF₂Q₁, —NR₂R₂, —COOR₂ and —CONR₂R₂, wherein Q₁ is selected from the group of aryl, heteroaryl, substituted heteroaryl, —OR₂, —COOR₃, —NR₂R₂, —SO₂R₇, —CONHSO₂R₃, and —CONHSO₂NR₂R₂;
Y is selected from the group of —COOR₂, —C(O)NR₂SO₂R₃, —C(O)NHSO₂NR₂R₂, —NR₂SO₂R₂, —SO₂NR₂R₂, —C₃₋₆ cycloalkyl-COOR₂, —C₂₋₆ alkenyl-COOR₂, —C₂₋₆ alkynyl-COOR₂, —C₁₋₆ alkyl-COOR₂, -alkylsubstituted C₁₋₆ alkyl, —COOR₂, CF₂—COOR₂, —NHC(O)(CH₂)ₙ—COOR₂, —SO₂NR₂C(O)R₂, -tetrazole, and —CONHOH, wherein n=1-6;
R₁ is selected from the group of: 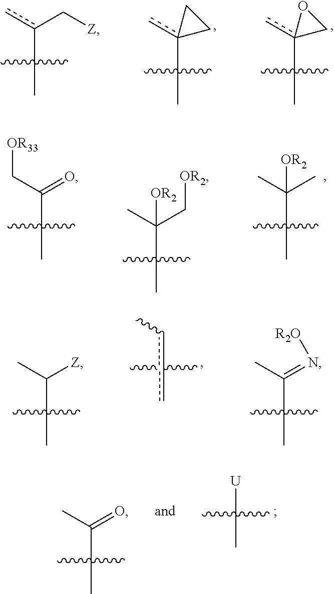
W is absent, or is —CH₂ or —CO;
Z is selected from the group of —NR₂₈R₂₉, —OR₃₀, —COOR₂, —CONR₁₈R₁₉, F, Cl, Br, and I;
U is selected from the group of —NR₂₈R₂₉, —OR₃₀, —COOR₂, —CONR₁₈R₁₉, F, Cl, Br, I, aryl and heteroaryl;
R₂ is selected from the group of —H, benzyl, —C₁₋₆ alkyl, -alkylsubstituted C₁₋₆ alkyl and -arylsubstituted C₁₋₆ alkyl;
R₃ is benzyl, —C₁₋₆ alkyl or -alkylsubstituted C₁₋₆ alkyl;
R₄ is selected from the group of —H, —C₁₋₆ alkyl, —C₁₋₆alkyl-C(OR₃)₂—C₃₋₆cycloalkyl, —C₁₋₆ substituted alkyl, —C₁₋₆ alkyl-C₃₋₆ cycloalkyl, —C₁₋₆ alkyl-Q₂, —C₁₋₆ alkyl-C₃₋₆ cycloalkyl-Q₂, aryl, heteroaryl, substituted heteroaryl, —COR₆, —COCOR₆, —SO₂R₇, —SO₂NR₂R₂, 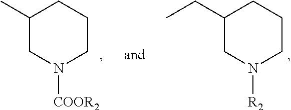
wherein Q₂ is selected from the group of heteroaryl, substituted heteroaryl, F, Cl, Br, I, —CF₃, —OR₂, —COOR₂, —NR₈R₉, —CONR₁₀R₁₁ and —SO₂R₇;
R₅ is selected from the group of —H, —C₁₋₆ alkyl, —C₃₋₆ cycloalkyl, —C₁₋₆ alkylsubstituted alkyl, —C₁₋₆alkyl-NR₈R₉, —COR₆, —COCOR₆, —SO₂R₇ and —SO₂NR₂R₂;
with the proviso that R₄ or R₅ cannot be —COR₆ or —COCOR₆ when W is CO;
with the further proviso that only one of R₄ or R₅ can be selected from the group of —COR₆, —COCOR₆, —SO₂R₇ and —SO₂NR₂R₂;
or when W is absent or is CH₂, then R₄ and R₅ can be taken together with the adjacent N to form 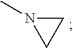
R₆ is selected from the group of —C₁₋₆ alkyl, —C₁₋₆ alkyl-substitutedalkyl, —C₃₋₆ cycloalkyl, —C₃₋₆ substitutedcycloalkyl-Q₃, —C₁₋₆ alkyl-Q₃, —C₁₋₆ alkyl-substitutedalkyl-Q₃, —C₃₋₆ cycloalkyl-Q₃, aryl-Q₃, —NR₁₃R₁₄, and —OR₁₅;
wherein Q₃ is selected from the group of aryl, heteroaryl, substituted heteroaryl, —OR₂, —COOR₂, —NR₈R₉, SO₂R₇, —CONHSO₂R₃, and —CONHSO₂NR₂R₂;
R₇ is selected from the group of —C₁₋₆ alkyl, —C₁₋₆ substituted alkyl, —C₃₋₆ cycloalkyl, —CF₃, aryl, and heteroaryl;
R₈ and R₉ are independently selected from the group of —H, —C₁₋₆ alkyl, —C₁₋₆ substituted alkyl, aryl, heteroaryl, substituted aryl, substituted heteroaryl, —C₁₋₆ alkyl-Q₂, and —COOR₃;
R₈ can also be —COOR_{S};
R₈ and R₉ can also be independently selected from the group of 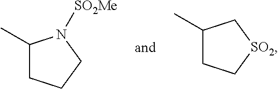
or R₈ and R₉ are taken together with the adjacent N to form a cycle selected from the grout of: 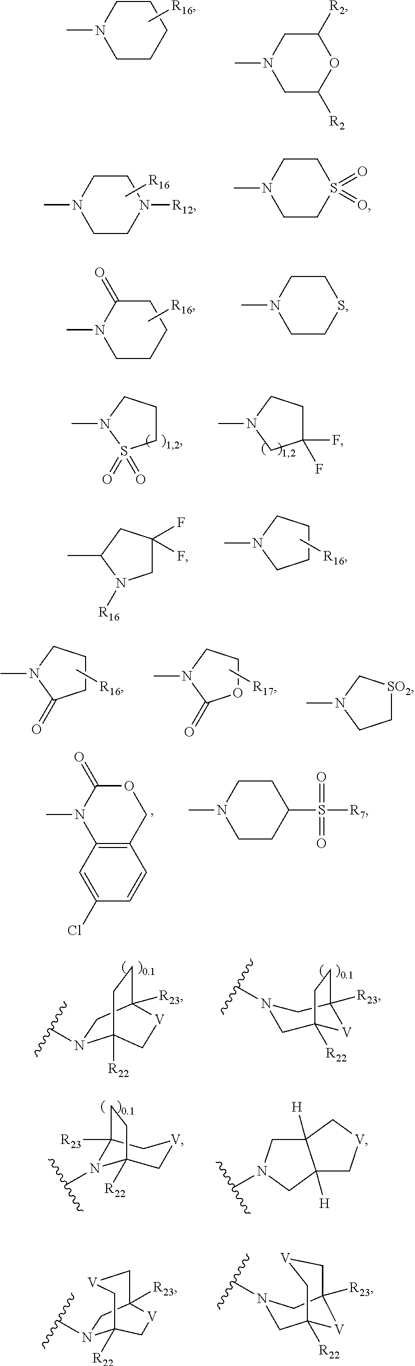 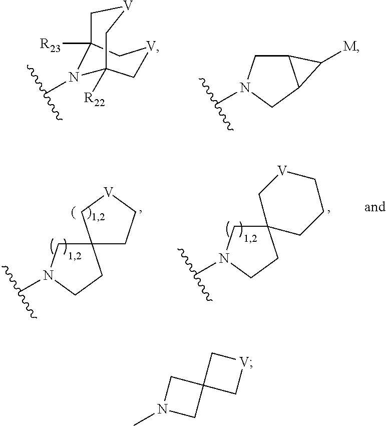
V is selected from the group of —CR₂₄R₂₅, —SO₂, —O and —NR₁₂;
M is selected from the group of —CHR₂₄R₂₅, —NR₂₆R₂₇, —SO₂R₇, —SO₂NR₃R₃ and —OH;
R₁₀ and R₁₁ are independently selected from the group of —H, —C₁₋₆ alkyl, —C₁₋₆ substituted alkyl and —C₃₋₆ cycloalkyl,
or R₁₀ and R₁₁ are taken together with the adjacent N to form a cycle such as 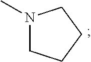
R₁₂ is selected from the group of —C₁₋₆ alkyl, —C₁₋₆ alkyl-OH; —C₁₋₆ alkyl, —C₁₋₆ substituted alkyl, —C₃₋₆ cycloalkyl, —COR₇, —COONR₁₈R₁₉, —SOR_{T}, and —SONR₂OR₂₁;
R₁₃ and R₁₄ are independently selected from the group of —H, —C₁₋₆ alkyl, —C₃₋₆ cycloalkyl, —C₁₋₆ substituted alkyl, —C₁₋₆ alkyl-Q₄, —C₁₋₆ alkyl-C₃₋₆ cycloalkyl-Q₄, —C₁₋₆ substituted alkyl-Q₄ and 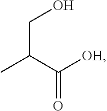
or R₁₃ and R₁₄ are taken together with the adjacent N to form a cycle selected from the group of: 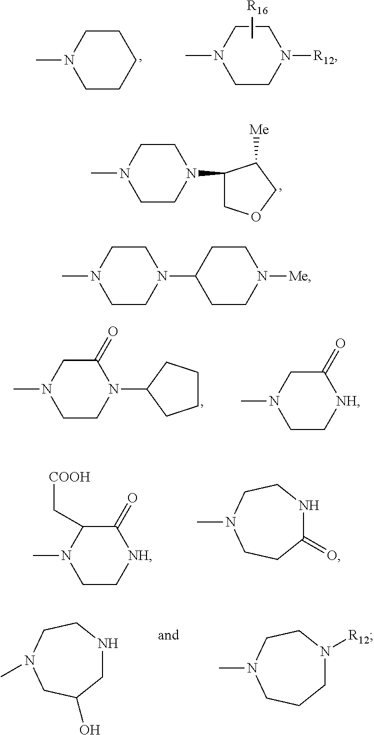
R₁₅ is selected from the group of —C₁₋₆ alkyl, —C₃₋₆ cycloalkyl, —C₁₋₆ substituted alkyl, —C₁₋₆ alkyl-Q₄, —C₁₋₆ alkyl-C₃₋₆ cycloalkyl-Q₄ and —C₁₋₆ substituted alkyl-Q₄, Q₄ is selected from the group of heteroaryl, substituted heteroaryl, —NR₂R₂, —CONR₂R₂, —COOR₂, —OR₂, and —SO₂R₃;
R₁₆ is selected from the group of —H, —C₁₋₆ alkyl, —NR₂R₂, and —COOR₃;
R₁₇ is selected from the group of —H, —C₁₋₆ alkyl, —COOR₃, and aryl;
R₁₈ and R₁₉ are independently selected from the group of H, —C₁₋₆ alkyl, —C₁₋₆ substituted alkyl, and —C₁₋₆ cycloalkyl;
R₁₈ can also be —COOR₃;
or R₁₈ and R₁₉ are taken together with the adjacent N to form a cycle selected from the group of 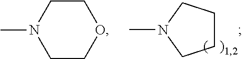
R₂₀ and R₂₁ are independently from the group of H, —C₁₋₆ alkyl, —C₁₋₆ substituted alkyl, —C₁₋₆ alkyl-Q₅, —C₁₋₆ cycloalkyl, aryl, substituted aryl, heteroaryl, and substituted heteroaryl, Q₅ is selected from the group of halogen and SO₂R₃ R₂₄ and R₂₅ are independently selected from the group of —H, —C₁₋₆ alkyl, -alkylsubstituted C₁₋₆ alkyl, —SO₂R₃, —SO₂NR₂R₂ or —OH, —NR₂R₂, —NR₂SO₂R₃, —NR₂COR₃ and —NR₂CONR₂R₂;
with the proviso that only one of R₂₄ and R₂₅ can be selected from the group of —OH, —NR₂R₂, —NR₂SO₂R₃, —NR₂COR₃ and —NR₂CONR₂R₂;
R₂₆ and R₂₇ are independently selected from the group of —H, —C₁₋₆ alkyl, -alkylsubstituted C₁₋₆ alkyl, —C₁₋₃ alkylaryl, —C₁₋₃alkylheteroaryl, —CO₂R₂ and —SO₂R₇;
with the proviso that only one of R₂₆ and R₂₇ can be selected from the group of —CO₂R₂ or —SO₂R₇;
R₂₈ and R₂₉ are independently selected from the group of —H, —C₁₋₆ alkyl, -alkylsubstituted C₁₋₆ alkyl, —C₃₋₆ cycloalkyl, —COOR₃; —COCF₃, R₂₈ can also be selected from —COOR_{S} and —CONR₁₈R₁₉;
or R₂₈ and R₂₉ are taken together with the adjacent N to form a cycle selected from the group of: 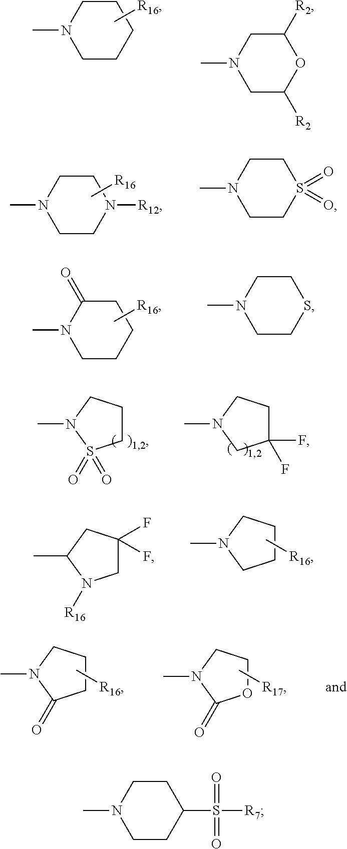
R₃₀ is selected from the group of H, —C₁₋₆ alkyl, -alkylsubstituted C₁₋₆ alkyl, —C₃₋₆ cycloalkyl, and —C₁₋₆ alkyl-Q₆;
wherein Q₆ is selected from the group of H, —OR₂, —COOR₂, —COCOOR₂, and —NR₃₁R₃₂;
R₃₁ and R₃₂ are independently selected from the group of —H, —C₁₋₆ alkyl, —C₁₋₆ substituted alkyl, —C₁₋₆ substituted alkyl-OR₂, and —COR₃,
or R₃₁ and R₃₂ are taken together with the adjacent N to form a cycle selected from the group of 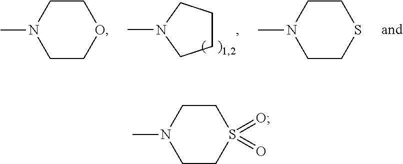
and
R₃₃ is selected from the group of —H, —C₁₋₆ alkyl, —C₁₋₆ substituted alkyl, and —C₁₋₆ substituted alkyl-Q₇,
wherein Q₇ is selected from the group of —COOR₂ and —COONR₂R₂.

2. A compound of claim 1, wherein X is phenyl.

3. A compound of claim 2, wherein A is —H.

4. A compound of claim 1, wherein Y is —COOR₂.

5. A compound of claim 4, wherein Y is —COOH.

6. A compound, including pharmaceutically acceptable salts thereof, which is selected from the group of:

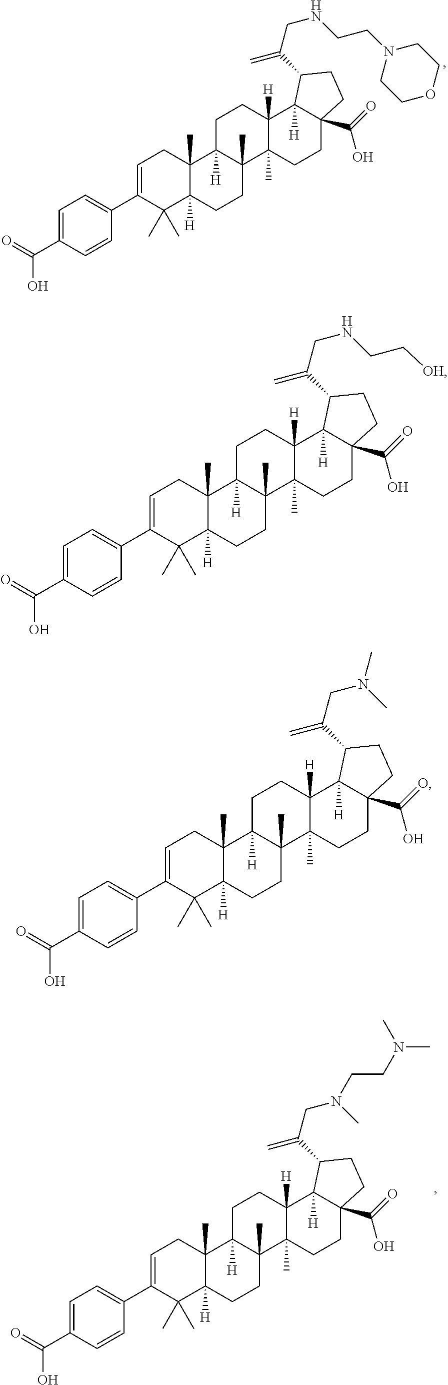

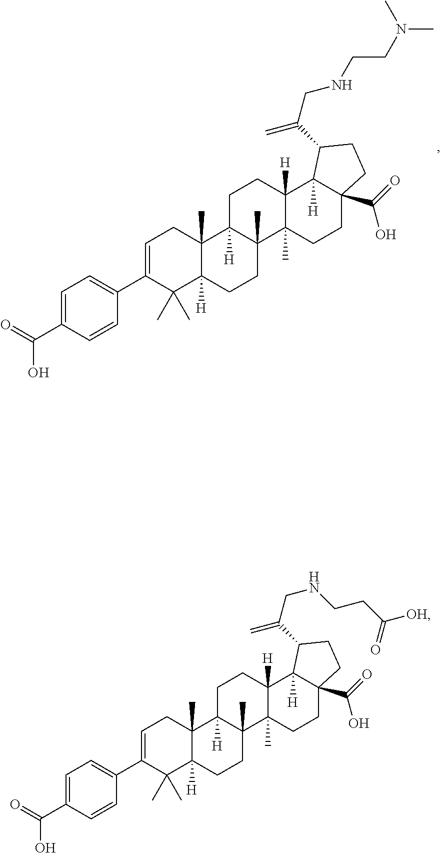

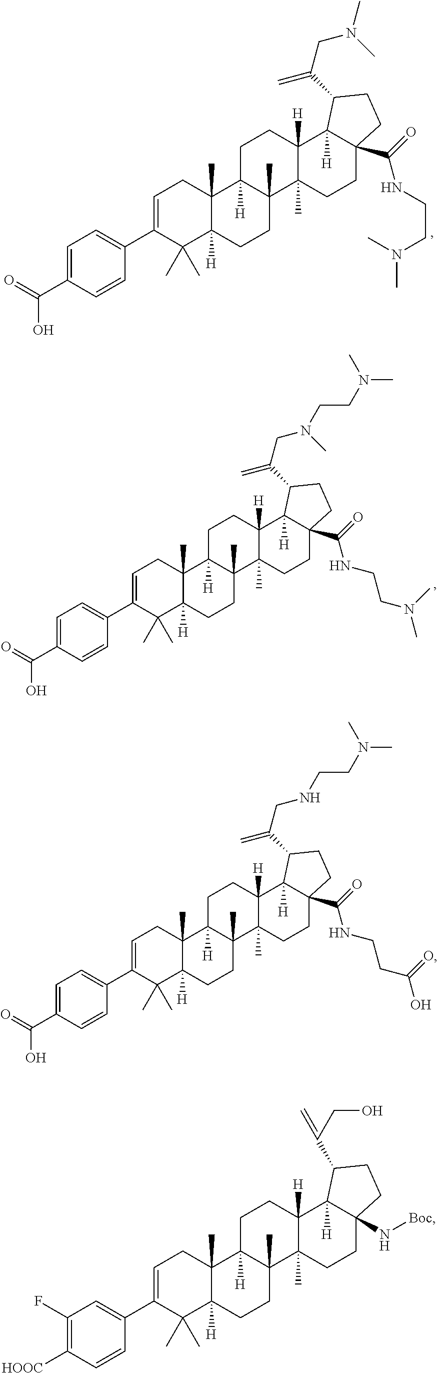

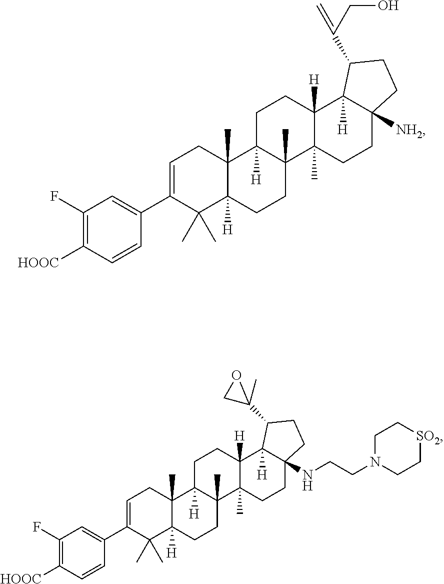

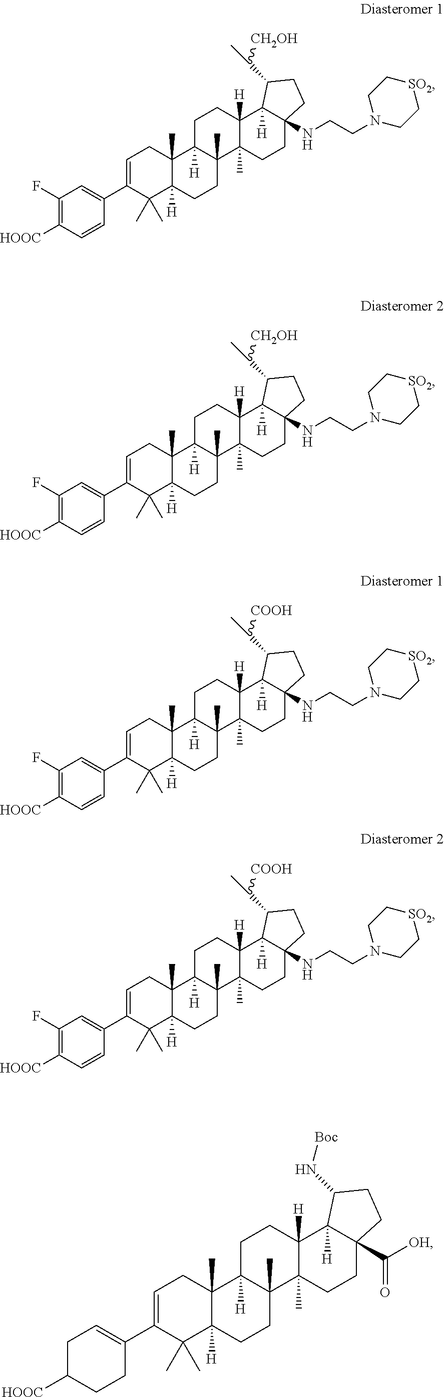

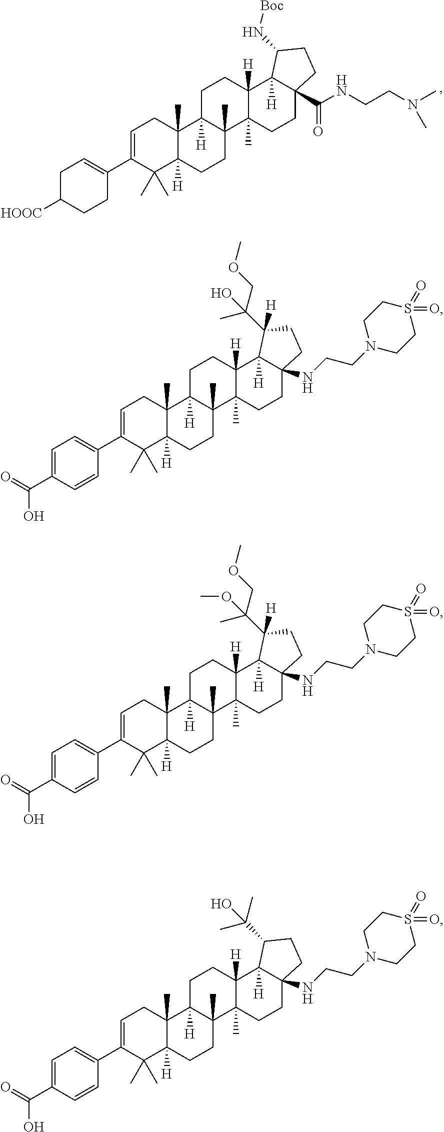

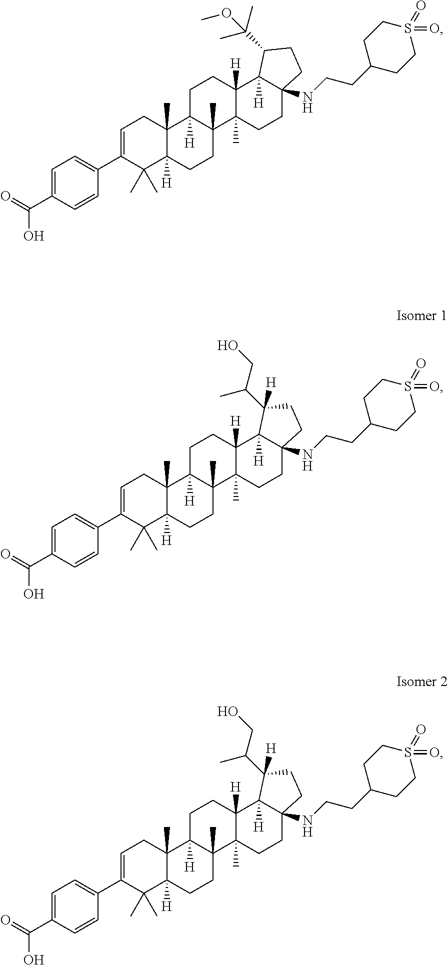

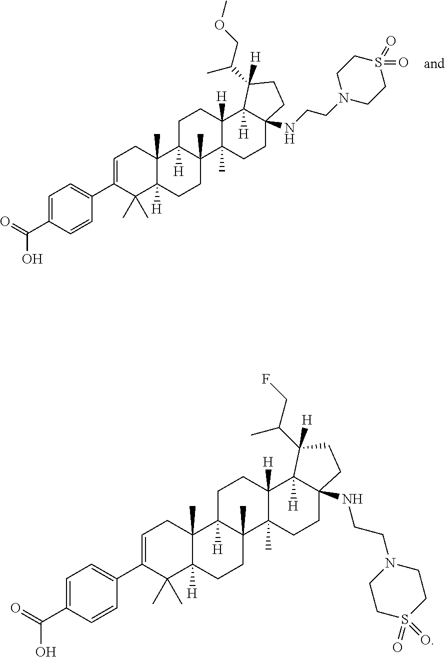

7. A pharmaceutical composition which comprises an antiviral effective amount of one or more of the compounds as claimed in claim 1, together with one or more pharmaceutically acceptable carriers, excipients or diluents.

8. The pharmaceutical composition of claim 7, useful for treating infection by HIV, which additionally comprises an antiviral effective amount of an AIDS treatment agent selected from the group of: (a) an AIDS antiviral agent; (b) an anti-infective agent; (c) an immunomodulator; and (d) another HIV entry inhibitor.

9. A method for treating a mammal infected with the HIV virus comprising administering to said mammal an antiviral effective amount of a compound as claimed in claim 1, and one or more pharmaceutically acceptable carriers, excipients or diluents.

10. A pharmaceutical composition which comprises an antiviral effective amount of one or more of the compounds as claimed in claim 6, together with one or more pharmaceutically acceptable carriers, excipients or diluents.

11. The pharmaceutical composition of claim 10, useful for treating infection by HIV, which additionally comprises an antiviral effective amount of an AIDS treatment agent selected from the group of: (a) an AIDS antiviral agent; (b) an anti-infective agent; (c) an immunomodulator; and (d) another HIV entry inhibitor.

12. A method for treating a mammal infected with the HIV virus comprising administering to said mammal an antiviral effective amount of a compound as claimed in claim 10, and one or more pharmaceutically acceptable carriers, excipients or diluents.
